(19) United States
(12) Patent Application Publication
(10) Pub. No.: US 2004/0010027 A1
(43) Pub. Date: Jan. 15, 2004

(54) Hydroxphenyl-pyrazole derivatives active as kinase inhibitors, process for their preparation and pharmaceutical comositions comprising them

(75) Inventors: Francesco Casuscelli, Parabiago (IT); Roberto D'Alessio, Cinisello Balsamo (IT); Eduard Felder, Mendrisio (IT); Anna Vulpetti, Brugherio (IT)

Correspondence Address: OBLON, SPIVAK, MCCLELLAND, MAIER & NEUSTADT, P.C., 1940 DUKE STREET, ALEXANDRIA, VA, 22314, US

(73) Assignee: PHARMACIA & UPJOHN SPA, Milan (IT)

(21) Appl. No.: 10/015,630

(22) Filed: Dec. 17, 2001

(51) Int. Cl. G01N 33/53; C07D 403/02; A61K 31/415; C07D 231/10

(52) U.S. Cl. 514/406; 435/7.1; 548/374.1; 548/364.1

(57) ABSTRACT

The present invention provides a method for treating diseases caused by and/or associated with an altered protein kinase activity which comprises administering to a mammal in need thereof an effective amount of a hydroxyphenyl-pyrazole derivative represented by formula (I):

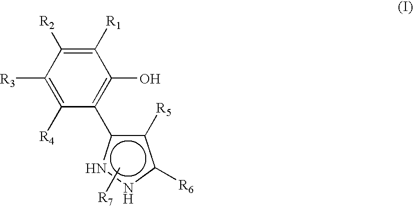

wherein R₁ to R₄ independently represent hydrogen or halogen atom or a group selected from hydroxy, NO₂, C₁-C₆ alkyl optionally substituted with halogen atom, aryl optionally substituted with halogen atom, aryl C₁-C₆ alkyl, C₃-C₇ cycloalkyl, saturated or unsaturated heterocyclyl, C₁-C₆ alkoxy, aryloxy, aryl C₁-C₆ alkoxy or NR₈R₉, wherein R₈ and R₉ independently represent hydrogen, C₁-C₆ alkyl, aryl, or a residue of formula COR₁₀, CONHR₁₀ or SO₂R₁₀ in which R₁₀ is hydrogen atom or C₁-C₆ alkyl, aryl, aryl C₁-C₆ alkyl, C₃-C₇ cycloalkyl or saturated or unsaturated heterocyclyl group; R₇ is a group of formula CONHR₁, CSNHR₁₀, SO₂R₁₀, COR₁₀ or COOR₁₀, in which R₁₀ is as defined above, and R₅ and R₆ independently represent hydrogen or a group selected from optionally substituted C₁-C₆ alkyl, optionally substituted aryl, aryl C₁-C₆ alkyl, C₃-C₇ cycloalkyl and saturated or unsaturated heterocyclyl; and pharmaceutically acceptable salts thereof. The invention also provides compounds of formula I, a library comprising at least two of them, a process for their preparation and the pharmaceutical compositions containing them, which are useful in the treatment of diseases caused by and/or associated with an altered protein kinase activity such as cancer, cell proliferative disorders, viral infections, autoimmune diseases and neurodegenerative disorders.

### BACKGROUND OF THE INVENTION

1. Field of the Invention

The present invention relates to pyrazole derivatives active as kinase inhibitors and, more in particular, it relates to hydroxyphenyl-pyrazole derivatives, to a process for their preparation, to pharmaceutical compositions comprising them and to their use as therapeutic agents, particularly in the treatment of diseases linked to disregulated protein kinases.

2. Discussion of the Background

The malfunctioning of protein kinases (PKs) is the hallmark of numerous diseases. A large share of the oncogenes and proto-oncogenes involved in human cancers code for PKs. The enhanced activities of PKs are also implicated in many non-malignant diseases such as benign prostate hyperplasia, familial adenomatosis, polyposis, neuro-fibromatosis, psoriasis, vascular smooth cell proliferation associated with atherosclerosis, pulmonary fibrosis, arthritis glomerulonephritis and post-surgical stenosis and restenosis. PKs are also implicated in inflammatory conditions and in the multiplication of viruses and parasites. PKs may also play a major role in the pathogenesis and development of neurodegenerative disorders.

For a general reference to PKs malfunctioning or disregulation see, for instance, Current Opinion in Chemical Biology 1999, 3, 459-465.

### SUMMARY OF THE INVENTION

The present inventors have now discovered that some hydroxyphenyl-pyrazoles are endowed with multiple protein kinase inhibiting activity and are thus useful in therapy in the treatment of diseases caused by and/or associated with disregulated protein kinases.

As such, it is an object of the invention to provide compounds which are useful as therapeutic agents against a host of diseases caused by a disregulated protein kinase activity.

It is another object to provide compounds endowed with multiple protein kinase inhibiting activity.

More specifically, the hydroxyphenyl-pyrazoles of this invention are useful in the treatment of a variety of cancers including, but not limited to: carcinoma such as bladder, breast, colon, kidney, liver, lung, including small cell lung cancer, esophagus, gall-bladder, ovary, pancreas, stomach, cervix, thyroid, prostate, and skin, including squamous cell carcinoma; hematopoietic tumors of lymphoid lineage, including leukemia, acute lymphocitic leukemia, acute lymphoblastic leukemia, B-cell lymphoma, T-cell-lymphoma, Hodgkin's lymphoma, non-Hodgkin's lymphoma, hairy cell lymphoma and Burkett's lymphoma; hematopoietic tumors of myeloid lineage, including acute and chronic myelogenous leukemias, myelodysplastic syndrome and promyelocytic leukemia; tumors of mesenchymal origin, including fibrosarcoma and rhabdomyosarcoma; tumors of the central and peripheral nervous system, including astrocytoma, neuroblastoma, glioma and schwannomas; other tumors, including melanoma, seminoma, teratocarcinoma, osteosarcoma, xeroderma pigmentosum, keratocanthoma, thyroid follicular cancer and Kaposi's sarcoma.

Due to the key role of PKs in the regulation of cellular proliferation, these hydroxyphenyl-pyrazoles are also useful in the treatment of a variety of cell proliferative disorders such as, for instance, benign prostate hyperplasia, familial adenomatosis, polyposis, neuro-fibromatosis, psoriasis, vascular smooth cell proliferation associated with atherosclerosis, pulmonary fibrosis, arthritis glomerulonephritis and post-surgical stenosis and restenosis.

The compounds of the invention can be useful in the treatment of Alzheimer's disease, as suggested by the fact that cdk5 is involved in the phosphorylation of tau protein (J. Biochem., 117,741-749, 1995).

The compounds of this invention, as modulators of apoptosis, may also be useful in the treatment of cancer, viral infections, prevention of AIDS development in HIV-infected individuals, autoimmune diseases and neurodegenerative disorders.

The compounds of this invention may be useful in inhibiting tumor angiogenesis and metastasis.

The compounds of the invention are useful as cyclin dependent kinase (cdk) inhibitors and also as inhibitors of other protein kinases such as, for instance, protein kinase C in different isoforms, Met, PAK-4, PAK-5, ZC-1, STLK-2, DDR-2, Aurora 1, Aurora 2, Bub-1, PLK, Chk1, Chk2, HER2, raf1, MEK1, MAPK, EGF-R, PDGF-R, FGF-R, IGF-R, VEGF-R, PI3K, weel kinase, Src, Abl, Akt, ILK, MK-2, IKK-2, Cdc7, Nek, and thus be effective in the treatment of diseases associated with other protein kinases.

Accordingly, the present invention provides a method for treating diseases caused by and/or associated with an altered protein kinase activity which comprises administering to a mammal in need thereof an effective amount of a hydroxyphenyl-pyrazole derivative represented by formula (I):

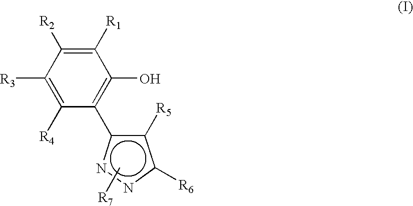

wherein R₁ to R₄ independently represent

hydrogen or halogen atom, hydroxy, nitro or NR₈R₉ group, wherein R₈ and R₉ independently represent hydrogen or an optionally substituted group selected from C₁-C₆ alkyl, aryl, aryl C₁-C₆ alkyl, C₃-C₇ cycloalkyl and saturated or unsaturated heterocyclyl group, or a residue of formula COR₁₀, CONHR₁₀ or SO₂R₁₀ in which R₁₀ is hydrogen atom or an optionally substituted group selected from C₁-C₆ alkyl, aryl, aryl C₁-C₆ alkyl, saturated or unsaturated C₃-C₇ cycloalkyl or saturated or unsaturated heterocyclyl group, or

an optionally substituted group selected from a straight or branched C₁-C₆ alkyl, aryl, aryl C₁-C₆ alkyl, a saturated or unsaturated C₃-C₇ cycloalkyl or cycloalkoxy group, saturated or unsaturated heterocyclyl, C₁-C₆ alkoxy, aryloxy, aryl C₁-C₆ alkoxy;

R₅ and R₆ independently represent hydrogen or an optionally substituted group selected from C₁-C₆ alkyl, aryl, aryl C₁-C₆ alkyl, C₃-C₇ cycloalkyl and saturated or unsaturated heterocyclyl,

R₇ is a substituent attached at one of the two nitrogen atoms of the pyrazole ring having the formula CONHR₁₀, CSNHR₁₀, SO₂R₁₀, COR₁₀ or COOR₁₀, in which R₁₀ is as above defined, or a pharmaceutically acceptable salt thereof.

In a preferred embodiment of the method described above, the disease caused by and/or associated with an altered protein kinase activity is selected from the group consisting of cancer, cell proliferative disorders, Alzheimer's disease, viral infections, auto-immune diseases and neurodegenerative disorders.

Specific types of cancer that may be treated according to the invention include carcinoma, squamous cell carcinoma, hematopoietic tumors of myeloid or lymphoid lineage, tumors of mesenchymal origin, tumors of the central and peripheral nervous system, melanoma, seminoma, teratocarcinoma, osteosarcoma, xeroderoma pignentosum, keratoxanthoma, thyroid follicular cancer and Kaposi's sarcoma.

In another preferred embodiment of the method described above, the cell proliferative disorder is selected from the group consisting of benign prostate hyperplasia, familial adenomatosis polyposis, neuro-fibromatosis, psoriasis, vascular smooth cell proliferation associated with atherosclerosis, pulmonary fibrosis, arthritis glomerulonephritis and post-surgical stenosis and restenosis. In addition, the method object of the present invention, provides tumor angiogenesis and metastasis inhibition.

The present invention also provides a hydroxyaryl-pyrazole derivative represented by formula (I):

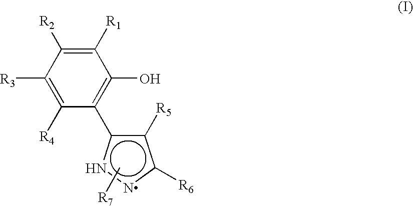

wherein R₁ to R₄ independently represent

hydrogen or halogen atom, hydroxy, nitro or NR₈R₉ group, wherein R₈ and R₉ independently represent hydrogen or an optionally substituted group selected from C₁-C₆ alkyl, aryl, aryl C₁-C₆ alkyl, C₃-C₇ cycloalkyl and saturated or unsaturated heterocyclyl group, or a residue of formula COR₁₀, CONHR₁₀ or SO₂R₁₀ in which R₁₀ is hydrogen atom or an optionally substituted group selected from C₁-C₆ alkyl, aryl, aryl C₁-C₆ alkyl, saturated or unsaturated C₃-C₇ cycloalkyl or saturated or unsaturated heterocyclyl group, or

an optionally substituted group selected from a straight or branched C₁-C₆ alkyl, aryl, aryl C₁-C₆ alkyl, a saturated or unsaturated C₃-C₆ cycloalkyl or cycloalkoxy group, saturated or unsaturated heterocyclyl, C₁-C₆ alkoxy, aryloxy, aryl C₁-C₆ alkoxy;

R₅ and R₆ independently represent hydrogen or an optionally substituted group selected from C₁-C₆ alkyl, aryl, aryl C₁-C₆ alkyl, C₃-C₇ cycloalkyl and saturated or unsaturated heterocyclyl,

R₇ is a substituent attached at one of the two nitrogen atoms of the pyrazole ring having the formula CONHR₁₀, CSNHR₁₀, SO₂R₁₀, COR₁₀ or COOR₁₀, in which R₁₀ is as above defined, with the following provisos:

when R₇ is CONH₂ or CSNH₂ and R₅ is H or CH₃, then R₆ is not H, CH₃ or phenyl group,

when R₇ is CO R₁₁₁ in which R′₁₁ is an optionally substituted C₁-C₆ alkyl, phenyl, or saturated or unsaturated heterocyclyl group, and R₅ is H or phenyl group, then R₆ is not H, CH₃ or an optionally substituted phenyl,

when R₇ is CO₂C(CH₃)₃ or CO₂CH₃ and R₅ is H, then R₆ is not an optionally substituted phenyl group;

or a pharmaceutically acceptable salt thereof.

The hydroxyphenyl-pyrazole derivatives of formula (I), object of the invention, are obtainable through a synthetic process comprising well known reactions carried out according to conventional techniques, as well as through a new and extremely versatile solid-phase combinatorial process, being both comprised within the scope of the invention. The present invention also provides a pharmaceutical composition comprising the hydroxyaryl-pyrazole derivatives of formula (I) and at least one pharmaceutically acceptable excipient, carrier or diluent.

A more complete appreciation of the invention and many of the attendant advantages thereof will be readily obtained as the same becomes better understood by reference to the following detailed description.

### DETAILED DESCRIPTION OF THE INVENTION

Some hydroxyphenyl-pyrazole derivatives structurally close to the compounds of formula (I) are known in the art. Among them are, as an example, certain pyrazoles active as antibacterial agents were studied (Mogilaiah et al, Indian J. Chem., B, 2001, 40B(1), p.43-48; Naik, S. M. et al. Asian J. Chem., 11(4), 1537-1539 and 1522-1524, 1999), and the reactivity of other derivatives was for instance discussed in Hamed, Ashraf et al., An. Quim. (1994), 90(5-6), 359-64 and in Rampey, Mary E.et al. Photochem. Photobiol., 70(2), 176-183, 1999.

The compounds of formula (I), object of the present invention, may have asymmetric carbon atoms and may therefore exist either as racemic admixtures or as individual optical isomers. Accordingly, all the possible isomers and their admixtures and of both the metabolites and the pharmaceutically acceptable bio-precursors (otherwise referred to as pro-drugs) of the compounds of formula (I), as well as any therapeutic method of treatment comprising them, are also within the scope of the present invention.

As stated above, as will be readily appreciated, the R₇ substituent on the pyrazole ring may be on one of the two nitrogens in the compounds of the invention:

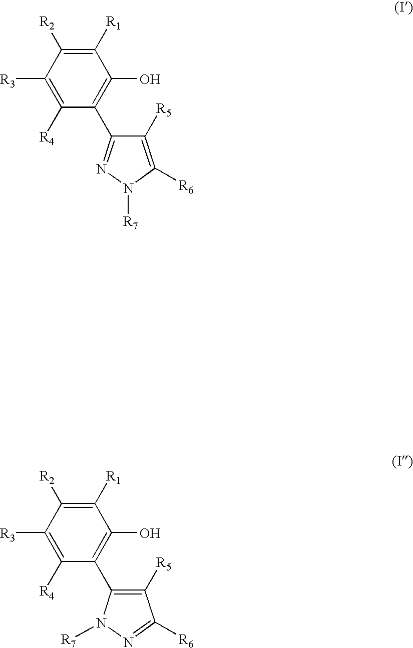

wherein R₁ to R₄, R₅, R₆ and R₇ are as defined above.

Accordingly, in the present invention and unless otherwise indicated, the general formula I comprises both the compounds of formula (I′) and the other of formula (I″). As used herein, unless otherwise specified, with the term straight or branched C₁-C₆ alkyl, we intend a group such as, for instance, methyl, ethyl, n-propyl, isopropyl, n-butyl, isobutyl, sec-butyl, tert-butyl, n-pentyl, neopentyl, n-hexyl, isohexyl, and the like.

With the term aryl we intend an aromatic carbocycle such as, for instance, phenyl, biphenyl, 1-naphthyl, 2-naphthyl, and the like.

With the term saturated or unsaturated C₃-C₇ cycloalkyl group we intend, for instance, cyclopropyl, cyclobutyl, cyclopentyl, cyclohexyl, cyclopentenyl, cyclohexenyl, and the like.

Unless otherwise specified, saturated or unsaturated cycloalkyl groups can be further condensed with 1 or 2 benzene rings are, for instance, 1,2,3,4-tetrahydro-naphthalene-2-yl, fluorene-9-yl, and the like.

The term “heterocyclyl” as used herein refers to a 3- to 7-membered, substituted or unsubstituted, saturated or unsaturated heterocyclyl ring, containing at least one heteroatom selected from O, S and N, any ring carbon may be oxidized as a carbonyl, and wherein said heterocyclyl ring may be optionally fused to a second 5- or 6-membered, saturated or unsaturated heterocyclyl ring, or to a C₃-C₇ cycloalkyl ring, or to a benzene or naphthalene ring. With the term 3- to 7-membered heterocycle with at least one heteroatom selected among nitrogen, oxygen or sulphur, we intend a saturated, partly unsaturated or fully unsaturated either aromatic or non aromatic heterocycle such as, for instance, thiophene, furan, pyrrole, imidazole, pyrazole, thiazole, isothiazole, oxazole, isoxazole, pyridine, pyrazine, pyrimidine, pyridazine, pyrrolidine, pyrroline, imidazolidine, imidazoline, piperidine, piperazine, morpholine, tetrahydrofuran, tetrahydropyran, tetrahydrothiopyran, and the like.

With the term optionally fused heterocycle and unless otherwise indicated we intend any of the above defined heterocycles further condensed, through any one of the available bonds, with 5- or 6-membered, saturated or unsaturated heterocyclyl ring, or to a C₃-C₇ cycloalkyl ring, or to a benzene or naphthalene ring such as, for instance, quinoline, isoquinoline, chroman, chromene, thionaphthene, indoline, and the like.

The term “aryl C₁-C₆ alkyl” refer to a straight or branched chain alkyl moiety having from 1 to 6 carbon atoms substituted with at least one aryl group as defined above, such as, for instance, benzyl, phenylethyl, benzhydryl, benzyloxy and the like.

With the term halogen atom, we intend fluoro, bromo, chloro or iodo atom.

The term “optionally substituted” means that the group may be substituted or unsubstituted; the substituents which may be present in the alkyl, cycloalkyl, aryl, arylcycloalkyl, alkoxy, aryloxy, cycloalkoxy, alkenyl or heterocyclyl groups in any of the above definitions of R₁-R₁₀ include the following:

halo (i.e., fluoro, bromo, chloro or iodo);

hydroxy;

nitro;

azido;

mercapto (i.e., —SH), and acetyl or phenylacetyl esters thereof (i.e., —SCOCH₃ and —SCOCH₂C₆H₅);

amino (i.e., —NH₂ or —NHR^{I} or —NR^{I}R^{II}, wherein R^{I} and R^{II}, which are the same or different, are straight or branched C₁-C₆ alkyl, phenyl, biphenyl (i.e., —C₆H₄—C₆H₅), or benzyl groups, optionally substituted by hydroxy, methoxy, methyl, amino, methylamino, dimethylamino, chloro or fluoro; or R^{I} and R^{II} taken together with the nitrogen atom to which they are attached form a heterocyclic ring such as morpholino, pyrrolidino, piperidino, pyperazino or N-methylpyperazino;

guanidino, i.e., —NHC(═NH)NH₂;

formyl (i.e. —CHO);

cyano;

carboxy (i.e. —COOH), or esters thereof (i.e., —COOR^{I}), or amides thereof (i.e., —CONH₂, —CONHR^{I} or —CONHR^{I}R^{II}), wherein R^{I} and R^{II} are as defined above, and including morpholino-amides, pyrrolidino-amides, and carboxymethylamides —CONHCH₂COOH;

sulfo (i.e., —SO₃H);

acyl, i.e., —C(O)R^{I}, wherein R^{I} is as defined above, including monofluoroacetyl, difluoroacetyl, trifluoroacetyl;

carbamoyloxy (i.e., —OCONH₂) and N-methylcarbamoyloxy;

acyloxy, i.e., —OC(O)R^{I} wherein R^{I} is as defined above, or formyloxy;

acylamino, i.e., —NHC(O)R^{I}, or —NHC(O)OR^{I}, wherein R^{I} is as defined above or is a group —(CH₂)ₜCOOH where t is 1, 2 or 3;

ureido, i.e., —NH(CO)NH₂, —NH(CO)NHR^{I}, —NH(CO)NR^{I}R^{II}, wherein R^{I} and R^{II} are as defined above, including —NH(CO)-(4-morpholino), —NH(CO)-(1-pyrrolidino), —NH(CO)-(1-piperazino), —NH(CO)-(4-methyl-1-piperazino);

sulfonamido, i.e., —NHSO₂R^{I} wherein R^{I} is as defined above;

a group —(CH₂)ₜCOOH, and esters and amides thereof, i.e., —(CH₂)ₜCOOR^{I} and —(CH₂)ₜCONH₂, —(CH₂)ₜCONHR^{I}, —(CH₂)ₜCONR^{I}R^{II}, wherein t, R^{I} and R^{II} are as defined above;

a group —NH(SO₂)NH₂, —NH(SO₂)NHR^{I}, —NH(SO₂)NR^{I}R^{II}, wherein R^{I} and R^{II} are as defined above, including —NH(SO₂)-(4-morpholino), —NH(SO₂)-(1-pyrrolidino), —NH(SO₂)-(1-piperazino), —NH(SO₂)-(4-methyl-1-piperazino);

a group —OC(O)OR^{I}, wherein R^{I} is as defined above;

a group —OR^{I}, wherein R^{I} is as defined above, including —OCH₂COOH;

a group —O—CH₂—O—, methylendioxy or —CH₂—CH₂—O—, ethylendioxy,

a group —SR^{I}, wherein R^{I} is as defined above, including —SCH₂COOH;

a group —S(O)R^{I}, wherein R^{I} is as defined above;

a group —S(O₂)R^{I}, wherein R^{I} is as defined above;

a group —SO₂NH₂, —SO₂NHR^{I}, or —SO₂NR^{I}R^{II}, wherein R^{I} and R^{II} are as defined above;

C₁-C₆ alkyl or C₂-C₆ alkenyl;

C₃-C₇ cycloalkyl;

substituted methyl selected from chloromethyl, fluoromethyl, difluoromethyl, trifluoromethyl, aminomethyl, N,N-dimethylaminomethyl, azidomethyl, cyanomethyl, carboxymethyl, sulfomethyl, carbamoylmethyl, carbamoyloxymethyl, hydroxymethyl, methoxycarbonylmethyl, ethoxycarbonylmethyl, tert-butoxycarbonylmethyl and guanidinomethyl.

Most preferred substituents are methoxy, trifluoromethyl, methylendioxy, dimethylamino, and ethoxycarbonyl groups.

When present, carboxy, hydroxy, mercapto and amino groups may be either free or in a protected form. Protected forms of said groups are any of those generally known in the art. Preferably, carboxy groups are protected as esters thereof, in particular methyl, ethyl, tert-butyl, benzyl, and 4-nitrobenzyl esters. Preferably, hydroxy groups are protected as silyl-ethers, ethers or esters thereof, in particular trimethyl silyl, tert-butyldiphenyl silyl, triethyl silyl, triisopropyl silyl or tert-butyldimethylsilyl ethers, methoxymethyl ethers, tetrahydropyranyl ethers, benzyl ethers, acetates or benzoates. Preferably, mercapto groups are protected as thioethers or thioesters, in particular tert-butyl thioethers, thioacetates or thiobenzoates. Preferably, amino groups are protected as carbamates, e.g. tert-butoxycarbonyl derivatives, or as amides, e.g. acetamides and benzamides.

Furthermore, hydrates, solvates of compounds of formula (I), and physiologically hydrolyzable derivatives (i.e., prodrugs) of compounds of formula (J) are included within the scope of the present invention.

With the term oxo we intend a carbonyl (>C═O) group.

With the term perfluorinated alkyl we intend any alkyl group as above defined being substituted by two or more fluorine atoms such as, for instance, trifluoromethyl, 2,2,2-trifluoroethyl, 1,1-difluoroethyl, and the like.

From all of the above, it is clear to the skilled man that any of the groups or substituents being defined, for instance, as arylalkyl, alkoxy, cycloalkoxy, aryloxy, arylalkyloxy and the like, have to be construed from the names of the groups from which they originate.

As an example, unless specifically noted otherwise, any arylalkyloxy group has to be intended as an alkyloxy wherein the alkyl moiety is substituted by at least one aryl, both aryl and alkyl being as above defined.

Pharmaceutically acceptable salts of the compounds of formula (I) are the acid addition salts with inorganic or organic, e.g. nitric, hydrochloric, hydrobromic, sulphuric, perchloric, phosphoric, acetic, trifluoroacetic, propionic, glycolic, lactic, oxalic, malonic, malic, maleic, tartaric, citric, benzoic, cinnamic, mandelic, methanesulphonic, isethionic and salicylic acid, as well as the salts with inorganic or organic bases, e.g. alkali or alkaline-earth metals, especially sodium, potassium, calcium or magnesium hydroxides, carbonates or bicarbonates, acyclic or cyclic amines, preferably methylamine, ethylamine, diethylamine, triethylamine or piperidine.

Within the compounds of formula (I) object of the invention, the R₇ group can be on one of the two nitrogen atoms of the pyrazole ring; preferably, the said R₇ is on the nitrogen in position meta with respect to the hydroxy-phenyl ring, as depicted in the formula I′ above.

Preferred compounds of formula (I) are the compounds wherein R₁, R₄ and R₅ are hydrogen atoms.

Other preferred compounds of formula (I) are the compounds wherein R₂ and R₃ are independently selected from

hydrogen or halogen atom,

an optionally substituted straight or branched C₁-C₆ alkyl or C₁-C₆ alkoxy, and —NR₈R₉ group, wherein R₈ and R₉ independently represent hydrogen atom or a residue of formula COR₁₀ wherein R₁₀ is an optionally substituted group selected from aryl and saturated or unsaturated heterocyclyl group.

Still more preferred, within this class, are the compounds of formula (I) wherein R₂ and R₃ are selected from hydrogen, chlorine or fluorine atom, optionally substituted straight or branched C₁-C₆ alkyl or C₁-C₆ alkoxy and NR₈R₉ group, wherein R₈ and R₉ independently represent hydrogen atom or a residue of formula COR₁₀ wherein R₁₀ is an optionally substituted group selected from phenyl, 1-naphthyl, 2-naphtyl, biphenyl, pyridyl, pyrazolyl, thienyl, isoxazolyl, thiazolyl, pyrazolyl, fluorene-9-yl, piperidine or tetrahydroquinoline.

Another preferred class of compounds of formula (I) are the compounds wherein R₆ is an optionally substituted group selected from C₁-C₆ alkyl, aryl, C₃-C₇ cycloalkyl and saturated or unsaturated heterocyclyl group.

Still more preferred, within this class, are the compounds of formula (I) wherein R₆ is an optionally substituted group selected from straight or branched C₁-C₆ alkyl, C₃-C₇ cycloalkyl, phenyl, 1-naphthyl, 2-naphtyl, biphenyl, pyridyl, pyrazolyl, thienyl, isoxazolyl, thiazolyl, pyrazolyl, fluorene-9-yl, piperidine or tetrahydroquinoline.

According to another preferred embodiment of the invention, the compounds of formula (I) are pyrazole derivatives wherein R₇ represents a group of formula CONHR₁₀, in which R₁₀ is hydrogen atom or an optionally substituted group selected from C₁-C₆ alkyl, aryl, aryl C₁-C₆ alkyl, C₂-C₈ alkenyl, C₃-C₇ cycloalkyl or saturated or unsaturated heterocyclyl group. More preferably, R₇ represents a group of formula CONHR₁₀, in which R₁₀ is C₁-C₆alkyl, phenyl, 1-naphthyl, 2-naphtyl, biphenyl, benzyl, allyl or phenethyl group.

Specific, not limiting, examples of the compounds of formula (I) of the invention, whenever appropriate in the form of pharmaceutically acceptable salts, are the following:

Ia. 5-Cyclopropyl-3-[2-hydroxy-4-(4-methoxy-benzoylamino)-phenyl]-pyrazole-1-carboxylic acid butylamide;

Ib. N-ethyl-3-(2-hydroxyphenyl)-5-pyridin-4-yl-1H-pyrazole-1-carboxamide;

Ic. 5-cyclopropyl-3-(2-hydroxyphenyl)-N-propyl-1H-pyrazole-1-carboxamide;

Id. ethyl N-{[5-cyclopropyl-3-(2-hydroxyphenyl)-1H-pyrazol-1-yl]carbonyl}glycinate;

Ie. N-allyl-5-cyclopropyl-3-(2-hydroxyphenyl)-1H-pyrazole-1-carboxamide;

If. N-cyclohexyl-5-cyclopropyl-3-(2-hydroxyphenyl)-1H-pyrazole-1-carboxamide;

Ig. 5-cyclopropyl-N-ethyl-3-(2-hydroxy-4-{[3-(trifluoromethyl)benzoyl]amino}phenyl)-1H-pyrazole-1-carboxamide;

Ih. ethyl-N-{[5-[2-(dimethylamino)ethyl]-3-(2-hydroxyphenyl)-1H-pyrazol-1-yl]carbonyl}glycinate;

Ii. 5-cyclopropyl-N-ethyl-3-(2-hydroxy-4-methoxyphenyl)-1H-pyrazole-1-carboxamide;

Ij. ethyl-N-{[5-cyclopropyl-3-(2-hydroxy-4-methoxyphenyl)-1H-pyrazol-1-yl]carbonyl}glycinate;

Ik. 5-cyclopropyl-N-ethyl-3-(2-hydroxy-5-methoxyphenyl)-1H-pyrazole-1-carboxamide;

Il. 5-cyclopropyl-N-ethyl-3-(2-hydroxy-5-methylphenyl)-1H-pyrazole-1-carboxamide;

Im. ethyl-N-{[5-cyclopropyl-3-(2-hydroxy-5-methylphenyl)-1H-pyrazol-1-yl]carbonyl}glycinate;

In. N-butyl-5-cyclopropyl-3-(2-hydroxy-4-methoxyphenyl)-1H-pyrazole-1-carboxamide:

Io. N-butyl-5-cyclopropyl-3-(2-hydroxy-5-methylphenyl)-1H-pyrazole-1-carboxamide;

Ip. N-(3-{1-[(butylamino)carbonyl]-5-cyclopropyl-1H-pyrazol-3-yl}-4-hydroxyphenyl)isonicotinamide;

Iq. N-(4-{1-[(butylamino)carbonyl]-5-cyclopropyl-1H-pyrazol-3-yl}-3-hydroxyphenyl)isonicotinamide;

Ir. 3-{4-[(1,3-benzodioxol-5-ylcarbonyl)amino]-2-hydroxyphenyl}-N-butyl-5-cyclobutyl-1H-pyrazole-1-carboxamide;

Is. 3-{4-[(1,3-benzodioxol-5-ylcarbonyl)amino]-2-hydroxyphenyl}-N-butyl-5-cyclopentyl-1H-pyrazole-1-carboxamide;

It. N-allyl-5-cyclopropyl-3-(4-fluoro-2-hydroxyphenyl)-1H-pyrazole-1-carboxamide;

Iu. 5-(3,4-dimethoxyphenyl)-N-ethyl-3-(2-hydroxy-5-methoxyphenyl)-1H-pyrazole-1-carboxamide;

Iv. N-allyl-5-[2-(dimethylamino)ethyl]-3-(2-hydroxy-5-methylphenyl)-1H-pyrazole-1-carboxamide;

Iw. 5-[2-(dimethylamino)ethyl]-3-(2-hydroxy-5-methoxyphenyl)-N-(2-phenylethyl)-1H-pyrazole-1-carboxamide;

Ix. ethyl-N-{[5-(3,4-dimethoxyphenyl)-3-(2-hydroxy-5-methoxyphenyl)-1H-pyrazol-1-yl]carbonyl}glycinate;

Iy. 5-cyclopropyl-N-ethyl-3-(4-fluoro-2-hydroxyphenyl)-1H-pyrazole-1-carboxamide

Iz. ethyl-N-{[3-(5-chloro-2-hydroxyphenyl)-5-cyclopropyl-1H-pyrazol-1-yl]carbonyl}glycinate;

Iaa. 5-cyclopropyl-3-(2-hydroxy-5-methoxyphenyl)-N-(2-phenylethyl)-1H-pyrazole-1-carboxamide;

Ibb. 5-cyclopropyl-N-ethyl-3-(2-hydroxyphenyl)-1H-pyrazole-1-carboxamide;

Icc. N-allyl-5-(3,4-dimethoxyphenyl)-3-(2-hydroxy-5-methoxyphenyl)-1H-pyrazole-1-carboxamide;

Idd. N-benzyl-5-(3,4-dimethoxyphenyl)-3-(2-hydroxy-5-methylphenyl)-1H-pyrazole-1-carboxaamide;

Iee. N-allyl-5-cyclopropyl-3-(2-hydroxy-5-methoxyphenyl)-1H-pyrazole-1-carboxamide;

Iff. N-benzyl-5-[2-(dimethylamino)ethyl]-3-(2-hydroxy-5-methoxyphenyl)-1H-pyrazole-1-carboxamide;

Igg. 5-cyclopropyl-3-(2-hydroxy-5-methylphenyl)-N-(2-phenylethyl)-1H-pyrazole-1-carboxamide;

Ihh. 5-[2-(dimethylamino)ethyl]-N-ethyl-3-(2-hydroxyphenyl)-1H-pyrazole-1-carboxamide;

Iii. Ethyl N-[(5-cyclobutyl-3-{2-hydroxy-4-[(3-methoxybenzoyl)amino]phenyl}-1H-pyrazol-1-yl)carbonyl]glycinate and

Ijj. Ethyl N-[(5-cyclopropyl-3-{2-hydroxy-4-[(3-methoxybenzoyl)amino]phenyl}-1H-pyrazol-1-yl)carbonyl]glycinate.

As formerly indicated, it is a further object of the invention a process for preparing the compounds of formula (I) and pharmaceutically acceptable salts thereof, which process comprises:

a) removing Q from a compound of formula (II)

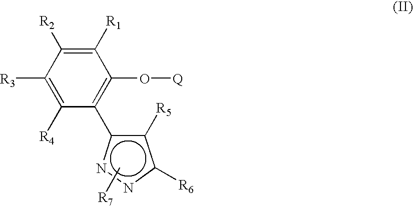

wherein R₁ to R₇ are as defined above and Q is a hydroxy-phenyl protecting group or a solid support, so as to obtain the compound of formula (I) as defined above and, if desired,

b) converting the resultant compound of formula (I) into another compound of formula (I) and/or into a salt or free form thereof.

The above process is an analogy process which can be carried out according to well known methods. It is clear to the person skilled in the art that if a compound of formula (I), prepared according to the above process, is obtained as an admixture of isomers, their separation into the single isomers of formula (I), carried out according to conventional techniques, is still within the scope of the present invention.

Likewise, the salification of a compound of formula (I) or the conversion of its salt into the free compound (I), carried out according to well-known procedures in the art, are still within the scope of the invention.

According to step a) of the process, the removal of Q as defined above from a compound of formula (II) is preferably carried out under acidic conditions, that is in the presence of suitable acids such as, for instance, hydrochloric, trifluoroacetic, methanesulphonic or p-toluensulphonic acid, as well as by using conventional acid ion exchange resins.

The reaction is carried out under conventional methods, for instance by using a solution of the acid, e.g. a 10% to 100% (v/v) of trifluoroacetic acid in dichlorometane, at a temperature ranging from about 0° C. to reflux, and for a suitable time, for instance from about 5 minutes to about 2 hours.

From the above it is clear to the skilled man that the optional conversion of a compound of formula (I) into another compound of formula (I) may be carried out by conventional methods. Likewise, a compound of formula (II) may be converted into another compound of formula (II) before undergoing deprotection. The conversion of a compound of formula I or II into another different compound of formula I or II may be carried out in several ways, depending on the meanings of the substituents and the presence of other substituents in the molecule.

For example, a conversion can be a hydrolysis, an oxidation, a reduction, a condensation with an appropriate reagent or a combination of these reactions.

Also the optional salification of a compound of formula (I) or the conversion of its salt into the free compound, as well as the separation of a mixture of isomers into the single isomers, may be all carried out by conventional methods.

The compound of formula (II) with a suitable hydroxy-phenyl protecting group or a solid support may be prepared by

i) reacting the compound of formula (E)

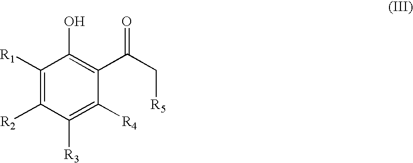

wherein R₁ to R₅ are as above defined, with a suitable hydroxy-phenyl protecting agent or a solid support, so as to obtain a compound of formula (IV)

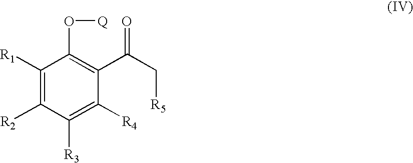

wherein R₁ to R₅ are as above defined and Q is the said hydroxy-phenyl protecting group or a solid support;

ia) optionally converting the compound of formula (IV) wherein one of R₁ to R₄ is an amino group into a different compound of formula (IV) wherein one of R₁ to R₄ is a group of formula NHCOR₁₀, NHCONHR₁₀ or NHSO₂R₁₀ wherein R₁₀ is as defined above, by reaction with any one of the compounds of the formula (V), (VI) or (VII):

R₁₀—COX (V);

R₁₀—N═C═O (VI);

R₁₀—SO₂X′ (II)

wherein R₁₀ is as above defined, X is hydroxy or a suitable leaving group and X′ is a suitable leaving group;

ii) reacting the compound of formula (IV) with a derivative of formula (VIII)

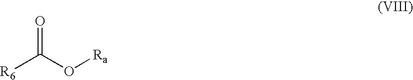

wherein Rₐ is a C₁-C₆ alkyl group, so as to obtain a compound of formula (IX)

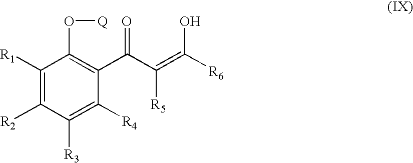

wherein Q, R₁ to R₆ are as above defined;

iii) reacting the compound of formula (IX) with hydrazine hydrate so as to obtain the compound of formula (X)

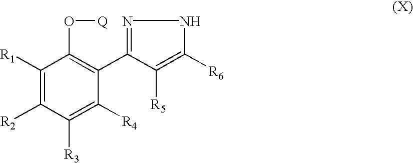

wherein Q, R₁ to R₆ are as above defined;

iv) reacting the compound of formula (X) with any one of the compounds of formula (V), (VI), (VII), (XI) or (XII):

R₁₀—COX (V);

R₁₀—N═C═O (VI);

R₁₀—SO₂X′ (VII);

R₁₀—N═C═S (XI)

R₁₀—OCOX (XII)

wherein R₁₀, X′ and X are as above defined, so as to obtain the compound of formula (II) as defined above.

According to step i) of the process, the compound of formula (III) is reacted with a suitable hydroxy-phenyl protecting agent such as, for instance, benzyl group; or with a solid support such as, for instance, a benzyloxy-polystyrene resin.

Preferably, the oxygen-phenol protecting agent or the solid support is selected from benzyl or a benzyloxy-polystyrene resin.

The reaction is carried out in the presence of a base such as cesium carbonate or potassium carbonate and a salt as tetrabutilamonium iodide or sodium iodide to enhance the solubility of carbonate, in a suitable solvent such as, for instance, N,N-dimethylacetamide or N,N-dimethylformamide, at a temperature ranging from about 0° C. to room temperature and for a suitable time, for instance from about 30 minutes to about 96 hours.

According to the optional step ia) of the process, the compound of formula (IV) is reacted with any one of the compounds of formula (V), (VI) or (VII), so as to obtain the corresponding different derivative of formula (IV). In this respect, it is clear to the skilled man that a carboxamido derivative of formula (IV) wherein one of R₁ to R₄ is NHCOR₁₀ is obtained through reaction with a compound of formula (V); an ureido derivative of formula (IV) wherein one of R₁ to R₄ is NHCONHR₁₀ is obtained through reaction with a compound of formula (VI) and a sulphonamido derivative of formula (VII) wherein one of R₁ to R₄ is NHSO₂R₁₀ is obtained through reaction with a compound of formula (VII). As formerly indicated, within the compound of formula (V) X is a hydroxy group or a suitable leaving group such as, for instance, a halogen atom. Preferably, in the reaction with a compound of formula (V) X is hydroxy, chlorine or bromine.

The reaction between a compound of formula (IV) and a carboxylic acid derivative of formula (V) wherein X is hydroxy group can be carried out in the presence of a coupling agent such as, for instance, benzotriazol-1-yloxytrispyrrolidino)phosphonium hexafluorophosphate carbo diimide, 1,3-dicyclohexylcarbodiimide, bromo-tris-pyrrolidino-phosphonium hexafluorophosphate, 1,3-diisopropylcarbodiimide, o-benzotriazol-1-yl-n,n,n′,n′-tetramethyluronium tetrafluoroborate, 1-(3-dimethylaminopropyl)-3-ethylcarbodiimide, N-cyclohexylcarbodiimide-N′-propyloxymethyl polystyrene or N-cyclohexylcarbodiimide-N′-methyl polystyrene, in a suitable solvent such as, for instance, dichloromethane, chloroform, tetrahydrofuran, diethyl ether, 1,4-dioxane, acetonitrile, toluene or N,N-dimethylformamide, at a temperature ranging from about −10° C. to reflux and for a suitable time ranging from about 30 minutes to about 96 hours.

The said reaction is optionally carried out in the presence of a suitable catalyst, for instance 4-dimethylaminopyridine, or in the presence of a further coupling agent such as N-hydroxybenzotriazole. The reaction between a compound of formula (IV) and a compound of formula (V) can also be carried out through a mixed anhydride method, that is by using an alkyl chloroformate such as ethyl, isobutyl, or isopropyl chloroformate, in the presence of a tertiary base such as triethylamine, N,N-diisopropylethylamine or pyridine, in a suitable solvent such as toluene, dichloromethane, chloroform, tetrahydrofuran, acetonitrile, diethyl ether, 1,4-dioxane or N,N-dimethylformamide, and at a temperature ranging from about −30° C. to room temperature.

The reaction between a compound of formula (IV) and a compound of formula (V) wherein X is a suitable leaving group, for instance chlorine or bromine, can be carried out in the presence of a tertiary base such as triethylamine, N,N-diisopropylethylamine or pyridine, in a suitable solvent such as toluene, dichloromethane, chloroform, diethyl ether, tetrahydrofuran, acetonitrile or N,N-dimethylformamide, and at a temperature ranging from about −10° C. to reflux.

The reaction of the compound of formula (IV) with an isocyanate derivative of formula (VI) can be carried out in the presence of a suitable catalyst, for instance 4-dimethylaminopyridine or a tertiary base such as N,N-diisopropylethylamine, in a suitable solvent such as toluene, diethyl ether, tetrahydrofuran, acetonitrile, or N,N-dimethylformamide, and at a temperature ranging from about 20° C. to 60° C.

As per step ia) of the process, within the compound of formula (VII) X′ is a suitable leaving group such as, for instance, a halogen atom. Preferably, X′ is chlorine or bromine. The reaction between a compound of formula (IV) and a sulphonyl derivative of formula (VI) can be carried out in the presence of a tertiary base such as triethylamine, N,N-diisopropylethylamine or pyridine, in a suitable solvent such as toluene, dichloromethane, chloroform, diethyl ether, tetrahydrofuran, acetonitrile or N,N-dimethylformamide, at a temperature ranging from about −10° C. to reflux.

According to step ii) of the process, the compound of formula (VIR) is reacted with the compound of formula (IV) under basic conditions, for instance in the presence of a base such as sodium hydryde or potassium ter-butoxide.

The reaction is carried out in a suitable solvent such as, for instance, N,N-dimethylformamide, N,N-dimethylacetamide at a temperature comprised from about 70° C. to 100° C. and for a suitable time, for instance from about 30 minutes to about 5 hours. According to step iii) of the process, the reaction between a compound of formula (IX) and hydrazine hydrate is carried out in a suitable solvent such as, for instance, tetrahydrofuran, acetonitrile, 1,4-dioxane, N,N-dimethylacetamide or N,N-dimethylformamide, and at a temperature ranging from about 60° C. to 90° C.

According to step iv) of the process, the reaction between a compound of formula (X) and a derivative of formula (V), (VI) or (VII) can be carried out as described in the detailed reaction description under step ia) above.

The reaction of a compound of formula (X) with an or isothiocyanate derivative of formula (XI) can be carried out in the presence of a suitable catalyst, for instance 4-dimethylaminopyridine or a tertiary base such as N,N-diisopropylethylamine, in a suitable solvent such as toluene, diethyl ether, tetrahydrofuran, acetonitrile, or N,N-dimethylformamide, and at a temperature ranging from about 20° C. to 60° C. The reaction of a compound of formula (X) with a derivative of formula (XII) can be carried out analogously to that between compound of formula (X) and the derivative of formula (V). As it will be really appreciated by the man skilled in the art, when preparing the compounds of formula (I) object of the invention, optional functional groups within both the starting materials or the intermediates thereof which could give rise to unwanted side reactions, need to be properly protected according to conventional techniques. Likewise, the conversion of these latter into the free deprotected compounds may be carried out according to known procedures.

The compounds of formula (III), (V), (VI), (VII), (VIII), (XI) and (XII) of the process are known or can be prepared according to known methods.

In addition to the above, it is also clear to the skilled man that the compounds of formula (I) of the invention can be advantageously prepared by combining the above described reactions in a combinatorial fashion, for example according to solid-phase-synthesis (SPS) techniques, so as to get a combinatorial chemical library of compounds of formula (I).

It is therefore a further object of the invention a library of two or more compounds of formula (I):

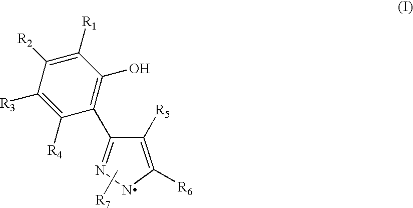

wherein R₁ to R₇ are as defined above.

As such, it is a further object of the present invention a compound of formula (I) which is obtainable, for instance through a combinatorial chemistry technique, as depicted in the following scheme, wherein Pol and Pol-Y are respectively a solid support and its activated form, X, X′, Rₐ, R₁, R₂, R₃, R₄, R₅, R₆, R₇ and R₁₀ are as defined above and the steps are carried out preferably under the reaction conditions set forth herein above. More preferably, one of R₂ and R₃ represents a group of formula NHCOR₁₀, NHCONHR₁₀ or NHSO₂R₁₀, wherein R₁₀ is as defined above, and the resultant compound has one of the following formula:

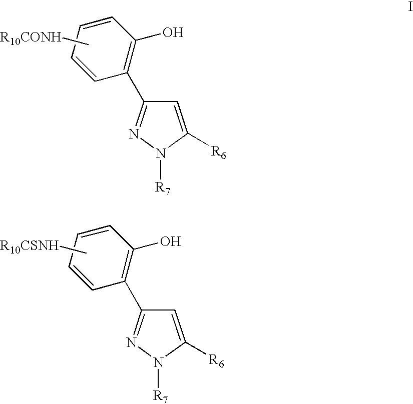

wherein R₆, R₇ and R₁₀ are as defined above.

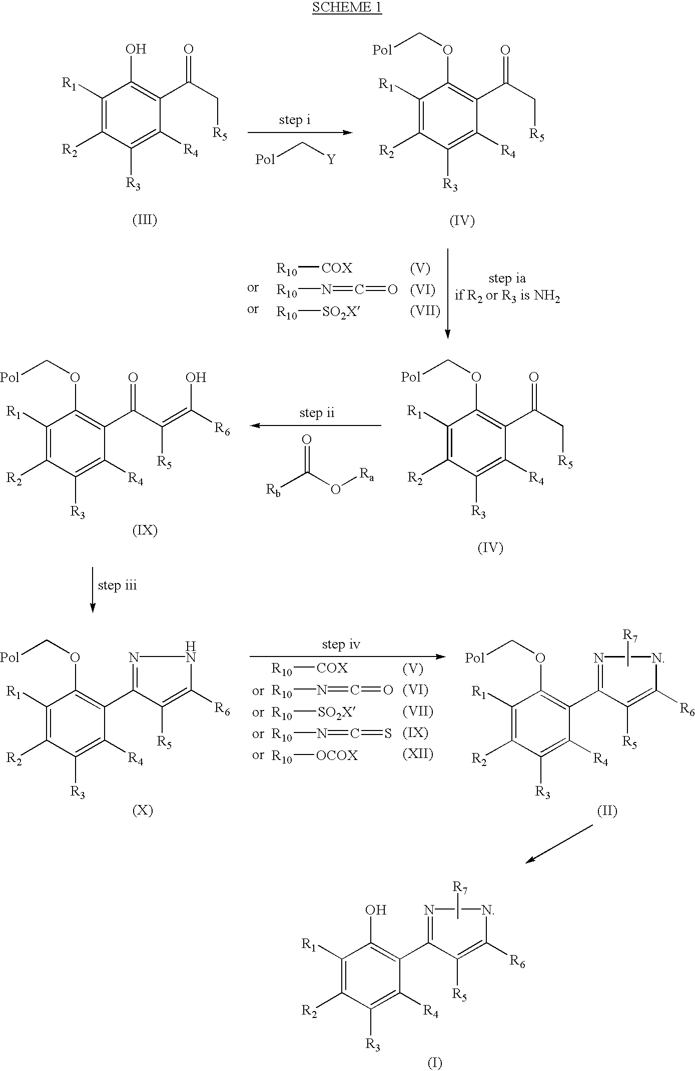

Each of the compounds of formula (IV)—obtained from the reaction between the solid support and a derivative of formula (III), as set forth in table I, is optionally reacted with any one of the derivatives of formula (V) or (VI) of table II and IV, and subsequently operating as per the process of the invention by reacting any one of the resultant derivatives of formula (IV) with any one of the derivatives of formula (VIII) as set forth in table III; reacting the resultant compounds (IX) with hydrazine hydrate and subsequently reacting the resultant compounds (X) with any one of the derivatives of formula (V) or (VI) as set forth in tables II and IV.

TABLE I
2-hydroxyacetophenones derivatives of formula (III)

| | |
|---|---|
| 1 | 4-amino-2-hydroxyacetophenone |
| 2 | 5-amino-2-hydroxyacetophenone |

TABLE II
Acyl chlorides derivatives of formula (V)

| | |
|---|---|
| 1 | 2-CHLOROBENZOYL CHLORIDE |
| 2 | 2,6-DICHLOROBENZOYL CHLORIDE |
| 3 | 2-METHOXYBENZOYL CHLORIDE |
| 4 | O-TOLUOYL CHLORIDE |
| 5 | 3-(TRIFLUOROMETHYL)BENZOYL CHLORIDE |
| 6 | 4-CHLOROBENZOYL CHLORIDE |
| 7 | 4-ETHOXYBENZOYL CHLORIDE |
| 8 | 4-TERT-BUTYLBENZOYL CHLORIDE |
| 9 | P-TOLUOYL CHLORIDE |
| 10 | 4-ETHYLBENZOYL CHLORIDE |
| 11 | ISOBUTYRYL CHLORIDE |
| 12 | ACETYL CHLORIDE |
| 13 | ISOVALERYL CHLORIDE |
| 14 | PROPIONYL CHLORIDE |
| 15 | CYCLOPROPANECARBONYL CHLORIDE |
| 16 | 2-FUROYL CHLORIDE |
| 17 | THIOPHENE-2-CARBONYL CHLORIDE |
| 18 | 2-QUINOXALOYL CHLORIDE |
| 19 | NICOTINOYL CHLORIDE HYDROCHLORIDE |
| 20 | ISONICOTINOYL CHLORIDE HYDROCHLORIDE |
| 21 | 3,4-METHYLENEDIOXYBENZOYL CHLORIDE |
| 22 | 2-ETHYLBUTYRYL CHLORIDE |
| 23 | BENZO[B]THIOPHENE-2-CARBONYL CHLORIDE |
| 24 | 2-CHLORONICOTINOYL CHLORIDE |
| 25 | 4-(TRIFLUOROMETHOXY)BENZOYL CHLORIDE |
| 26 | ISOXAZOLE-5-CARBONYL CHLORIDE |
| 27 | 1-METHYLPYRROLE-2-CARBONYL CHLORIDE |

TABLE III
Esters derivatives of formula (VIII)

| | |
|---|---|
| 1 | ETHYL CYCLOPROPANECARBOXYLATE |
| 2 | ETHYL CYCLOBUTANECARBOXYLATE |
| 3 | METHYL CYCLOPENTANECARBOXYLATE |

TABLE IV
Isocyanates derivatives of formula (VI)

| | |
|---|---|
| 1 | ISOPROPYL ISOCYANATE |
| 2 | ETHYL ISOCYANATE |
| 3 | ALLYL ISOCYANATE |
| 4 | N-PROPYL ISOCYANATE |
| 5 | BENZYL ISOCYANATE |
| 6 | PHENETHYL ISOCYANATE |
| 7 | 2-METHYLBENZYL ISOCYANATE |
| 8 | 3-METHYLBENZYL ISOCYANATE |
| 9 | 4-METHYLBENZYL ISOCYANATE |
| 10 | 2-CHLOROBENZYL ISOCYANATE |
| 11 | 4-FLUOROBENZYL ISOCYANATE |
| 12 | 3,4-DICHLOROBENZYL ISOCYANATE |
| 13 | 4-METHOXYBENZYL ISOCYANATE |
| 14 | SEC-BUTYL ISOCYANATE |
| 15 | 2-FLUOROBENZYL ISOCYANATE |
| 16 | 3-FLUOROBENZYL ISOCYANATE |
| 17 | CYCLOPROPYL ISOCYANATE |

All of the compounds of formula (I) which are prepared according to combinatorial chemistry techniques, for instance as reported in the examples, whenever appropriate in the form of pharmaceutically acceptable salts, are herewith conveniently indicated and defined as “products by process”, that is as compounds of formula (I) which are obtainable through a given process.

As such, it is a further object of the present invention a compound of formula (I) which is obtainable, for instance through a combinatorial chemistry technique, by reacting each of the derivatives obtained from a compound of formula (III), as set forth in table I, with any one of the carboxylic acid derivatives of formula (VIII), as set forth in table II, and by subsequently operating as per the process of the invention.

It is a further object of the present invention a compound of formula (I) which is obtainable, for instance through a combinatorial chemistry technique, by reacting each of the derivatives obtained from a compound of formula (II) wherein R₂ or R₃ is an amino group, as set forth in table I, with any one of the acyl chloride or isocyanate derivatives of formula (V) and (VI), as set forth in tables II and IV, and by subsequently operating as per the process of the invention.

Pharmacology

The compounds of formula (I) are active as protein kinase inhibitors and are therefore useful, for instance, to restrict the unregulated proliferation of tumor cells.

In therapy, they may be used in the treatment of various tumors such as, for instance, carcinomas, e.g. mammary carcinoma, lung carcinoma, bladder carcinoma, colon carcinoma, ovary and endometrial tumors, sarcomas, e.g. soft tissue and bone sarcomas, and the hematological malignancies such as, e.g., leukemias.

In addition, the compounds of formula (I) are also useful in the treatment of other cell proliferative disorders such as psoriasis, vascular smooth cell proliferation associated with atherosclerosis and post-surgical stenosis and restenosis and in the treatment of Alzheimer's disease.

The inhibiting activity of putative protein kinase inhibitors and the potency of selected compounds was determined through a method of assay based on the use of the MultiScreen-PH 96 well plate (Millipore), in which a phosphocellulose filter paper was placed at each well bottom allowing binding of positive charged substrate after a washing/filtration step.

When a radioactivity labeled phosphate moiety was transferred by the ser/threo kinase to the filter-bound histone, light emitted was measured in a scintillation counter.

Inhibition Assay of cdk2/Cyclin A activity

Kinase reaction: 1.5 μM histone H1 substrate, 25 μM ATP (0.2 uCi P33□-ATP), 30 ng of baculovirus co-expressed cdk2/Cyclin A, 10 μM inhibitor in a final volume of 100 μl buffer (TRIS HCl 10 mM pH 7.5, MgCl₂ 10 mM, 7.5 mM DTT) were added to each well of a 96 U bottom well plate. After 10 min at 37° C. incubation, reaction was stopped by 20 μl EDTA 120 mM.

Capture: 100 μl were transferred from each well to MultiScreen plate, to allow substrate binding to phosphocellulose filter. Plates were then washed 3 times with 150 μl/well PBS Ca++/Mg++ free and filtered by MultiScreen filtration system.

Detection: filters were allowed to dry at 37° C., then 100 μl/well scintillant were added and 33P labeled histone H1 was detected by radioactivity counting in the Top-Count instrument.

Results: data were analyzed and expressed as % inhibition referred to total activity of enzyme (=100%).

All compounds showing inhibition ≧50% were further analyzed in order to study and define potency (IC50) as well as the kinetic-profile of inhibitor through Ki calculation.

IC50 determination: the protocol used was the same described above, where inhibitors were tested at different concentrations ranging from 0.0045 to 10 μM. Experimental data were analyzed by the computer program GraphPad Prizm using the four parameter logistic equation:

y=bottom+(top−bottom)/(1+10\hat{ }((logIC50−x)*slope))

where x is the logarithm of the inhibitor concentration, y is the response; y starts at bottom and goes to top with a sigmoid shape.

Ki calculation: either the concentration of ATP and histone H1 substrate were varied: 4, 8, 12, 24, 48 μM for ATP (containing proportionally diluted P³³γ-ATP) and 0.4, 0.8, 1.2, 2.4, 4.8 μM for histone were used in absence and presence of two different, properly chosen inhibitor concentrations.

Experimental data were analyzed by the computer program “SigmaPlot” for Ki determination, using a random bireactant system equation:

$$v = \frac{V max \frac{( A ) ( B )}{aKAKB}}{1 + \frac{( A )}{KA} + \frac{( B )}{KB} + \frac{( A ) ( B )}{aKAKB}}$$

where A=ATP and B=histone H1.

In addition the selected compounds have been characterized on a panel of ser/threo kinases strictly related to cell cycle (cdk2/cyclin E, cdk1/cyclin B1, cdk4/Cyclin D1), and also for specificity on MAPK, PKA, EGFR, IGF1-R, Cdc7/dbf4 and aurora-2.

Inhibition assay of cdk2/Cyclin E Activity

Kinase reaction: 1.5 μM histone H1 (Sigma #H-5505) substrate, 25 μM ATP (0.2 □Ci P³³γ-ATP), 15 ng of baculovirus co-expressed cdk2/GST-Cyclin E, suitable concentrations of inhibitor in a final volume of 100 μl buffer (TRIS HCl 10 mM pH 7.5, MgCl₂ 10 mM, 7.5 mM DTT+0.2 mg/ml BSA) were added to each well of a 96 U bottom well plate. After 10 min at 37° C. incubation, reaction was stopped by 20 μl EDTA 120 mM.

Capture: 100 μl were transferred from each well to MultiScreen plate, to allow substrate binding to phosphocellulose filter. Plates were then washed 3 times with 150 □l/well PBS Ca⁺⁺/Mg⁺⁺ free and filtered by MultiScreen filtration system.

Detection: filters were allowed to dry at 37° C., then 100 μl/well scintillant were added and ³³P labeled histone H1 was detected by radioactivity counting in the Top-Count instrument.

Inhibition Assay of cdkl/Cyclin B1 Activity

Kinase reaction: 1.5 μM histone H1 (Sigma # H-5505) substrate, 25 μM ATP (0.2 μCi P³³γ-ATP), 30 ng of baculovirus co-expressed cdkl/Cyclin B1, suitable concentrations of inhibitor in a final volume of 100 μl buffer (TRIS HCl 10 mM pH 7.5, MgCl₂ 10 mM, 7.5 mM DTT+0.2 mg/ml BSA) were added to each well of a 96 U bottom well plate. After 10 min at 37° C. incubation, reaction was stopped by 20 μl EDTA 120 mM.

Capture: 100 μl were transferred from each well to MultiScreen plate, to allow substrate binding to phosphocellulose filter. Plates were then washed 3 times with 150 μl/well PBS Ca⁺⁺/Mg⁺⁺ free and filtered by MultiScreen filtration system.

Detection: filters were allowed to dry at 37° C., then 100 μl/well scintillant were added and ³³P labeled histone H1 was detected by radioactivity counting in the Top-Count instrument.

Inhibition assay cdk4/Cyclin D1 Activity

Kinase reaction: 0,4 μM μM mouse GST-Rb(769-921) (# sc-4112 from Santa Cruz) substrate, 10 μM ATP (0.5 μCi P³³γ-ATP), 100 ng of baculovirus expressed GST-cdk4/GST-Cyclin D1, suitable concentrations of inhibitor in a final volume of 50 μl buffer (TRIS HCl 10 mM pH 7.5, MgCl₂ 10 mM, 7.5 mM DTT+0.2 mg/ml BSA) were added to each well of a 96 U bottom well plate. After 40 min at 37° C. incubation, reaction was stopped by 20 μl EDTA 120 mM.

Capture: 60 μl were transferred from each well to MultiScreen plate, to allow substrate binding to phosphocellulose filter. Plates were then washed 3 times with 150 □l/well PBS Ca⁺⁺/Mg⁺⁺ free and filtered by MultiScreen filtration system.

Detection: filters were allowed to dry at 37° C., then 100 μl/well scintillant were added and ³³P labeled Rb fragment was detected by radioactivity counting in the Top-Count instrument.

Inhibition assay of MAPK Activity

Kinase reaction: 10 μM MBP (Sigma #M-1891) substrate, 25 μM ATP (0.2 tCi P³³γ-ATP), 25 ng of bacterially expressed GST-MAPK (Upstate Biotechnology #14-173), suitable concentrations of inhibitor in a final volume of 100 μl buffer (TRIS HCl 10 mM pH 7.5, MgCl₂ 10 mM, 7.5 mM DTT+0.1 mg/ml BSA) were added to each well of a 96 U bottom well plate. After 15 min at 37° C. incubation, reaction was stopped by 20 μl EDTA 120 mM.

Capture: 100 μl were transferred from each well to MultiScreen plate, to allow substrate binding to phosphocellulose filter. Plates were then washed 3 times with 150 μl/well PBS Ca⁺⁺/Mg⁺⁺ free and filtered by MultiScreen filtration system.

Detection: filters were allowed to dry at 37° C., then 100 μl/well scintillant were added and ³³P labeled MBP was detected by radioactivity counting in the Top-Count instrument.

Inhibition Assay of PKA Activity

Kinase reaction: 10 μM histone Hi (Sigma #H-5505) substrate, 10 μM ATP (0.2 μCi P³³γ-ATP), 1U of bovine heart PKA (Sigma # 2645), suitable concentrations of inhibitor in a final volume of 100 μl buffer (TRIS HCl 10 mM pH 7.5, MgCl₂ 10 mM, 7.5 mM DTT+0.2 mg/ml BSA) were added to each well of a 96 U bottom well plate. After 5 min at 37° C. incubation, reaction was stopped by 20 μl EDTA 120 mM.

Capture: 100 μl were transferred from each well to MultiScreen plate, to allow substrate binding to phosphocellulose filter. Plates were then washed 3 times with 150 μl/well PBS Ca⁺⁺/Mg⁺⁺ free and filtered by MultiScreen filtration system.

Detection: filters were allowed to dry at 37° C., then 100 μl/well scintillant were added and ³³P labeled histone H1 was detected by radioactivity counting in the Top-Count instrument.

Inhibition Assay of EGFR Activity

Kinase reaction: 25 nM in house biotinylated PolyGluTyr (Sigma # 0275) substrate, 2,5 μM ATP (0.3 μCi P³³γ-ATP), 80 ng baculovirus expressed GST-EGFR, suitable concentrations of inhibitor in a final volume of 100 μl buffer (Hepes 50 mM pH 7,5, MnCl₂— MgCl₂ 3 mM, 1 mM DTT+3 μM NaVO3, 0.1 mg/ml BSA) were added to each well of a 96 U bottom well plate. After 5 min. at 37° C. incubation, reaction was stopped by 20 μl EDTA 120 mM.

Capture: 100 μl were transferred from each well to streptavidin-Flashplate, to allow biotinylated-substrate binding to plate. Plates were then washed 3 times with 150 μl/well PBS Ca⁺⁺/Mg⁺⁺ free.

Detection: radioactivity counting in the Top-Count instrument.

Inhibition Assay of IGF1-R Activity

The inhibition assay of IGFI—R activity was performed according to the following protocol.

Kinase reaction: 10 μM biotinylated MBP (Sigma cat. #M-1891) substrate, 0-20 μM inhibitor, 6 μM cold ATP, 2 nM ³³P-ATP, and 22.5 ng IGF1-R (pre-incubated for 30 min at room temperature with cold 60 μM cold ATP) in a final volume of 30 μl buffer (50 mM HEPES pH 7.9, 3 mM MnCl₂, 1 mM DTT, 3 μM NaVO₃) were added to each well of a 96 U bottom well plate. After incubation for 35 min at room temperature, the reaction was stopped by addition of 100 μl PBS buffer containing 32 mM EDTA, 500 μM cold ATP, 0.1% Triton X100 and 10 mg/ml streptavidin coated SPA beads. After 15 min incubation, 110 μl of suspension were withdrawn and transferred into 96-well OPTIPLATEs containing 100 μl of 5M CsCl. After 4 hours, the plates were read for 2 min in a Packard TOP-Count radioactivity reader.

Results: Experimental data were analyzed with the program GraphPad Prizm.

In addition, the inhibiting activity of putative protein kinase inhibitors and the potency of selected compounds was also determined through a method of assay based on the use of a SPA (Scintillation Proximity Assay) 96 well plate assay. The assay is based on the ability of streptavidin coated SPA beads to capture a biotinylated peptide derived from a phosphorylation site of histone.

When a radioactivity labeled phosphate moiety was transferred by the ser/threo kinase to the biotinylated histone peptide, light emitted was measured in a scintillation counter.

Inhibition Assay of cdk5/p25 Activity

The inhibition assay of cdk5/p25 activity was performed according to the following protocol.

Kinase reaction: 1.0 μM biotinylated histone peptide substrate, 0.25 uCi P33 g-ATP, 4 nM cdk5/p25 complex, 0-100 μM inhibitor in a final volume of 100 μl buffer (Hepes 20 mM pH 7.5, MgCl2 15 mM, 1 mM DTT) were added to each well of a 96 U bottom well plate. After 20 min at 37° C. incubation, the reaction was stopped by the addition of 500 ug SPA beads in phosphate-buffered saline containing 0.1% Triton X-100, 50 uM ATP and 5 mM EDTA. The beads were allowed to settle, and the radioactivity incorporated in the ³³P-labelled peptide was detected in a Top Count scintillation counter.

Results: Data were analyzed and expressed as % Inhibition using the formula:

100×(1−(Unknown−Bkgd)/(Enz. Control−Bkgd))

IC50 values were calculated using a variation of the four parameter logistics equation:

Y=100/[1+10\hat{ }((LogEC50−X)*Slope)]

Where X=log(uM) and Y=% Ihibition.

Inhibition Assay of Cdc7/dbf4 Activity

The inhibition assay of Cdc7/dbf4 activity was performed according to the following protocol.

The Biotin-MCM2 substrate is trans-phosphorylated by the Cdc7/Dbf4 complex in the presence of ATP traced with γ³³-ATP. The phosphorylated Biotin-MCM2 substrate is then captured by Streptavidin-coated SPA beads and the extent of phosphorylation evaluated by β counting.

The inhibition assay of Cdc7/dbf4 activity was performed in 96 wells plate according to the following protocol.

To each well of the plate were added:

10 μl substrate (biotinylated MCM2, 6 μM final concentration)

10 μl enzyme (Cdc7/Dbf4, 12.5 nM final concentration)

10 μl test compound (12 increasing concentrations in the nM to μM range to generate a dose-response curve)

10 μl of a mixture of cold ATP (10 μM final concentration) and radioactive ATP (1/2500 molar ratio with cold ATP) was then used to start the reaction which was allowed to take place at 37° C.

Substrate, enzyme and ATP were diluted in 50 mM HEPES pH 7.9 containing 15 mM MgCl₂, 2 mM DTT, 3 μM NaVO₃, 2 mM glycerophosphate and 0.2 mg/ml BSA. The solvent for test compounds also contained 10% DMSO.

After incubation for 20 minutes, the reaction was stopped by adding to each well 100 □l of PBS pH 7.4 containing 50 mM EDTA, 1 mM cold ATP, 0.1% Triton X100 and 10 mg/ml streptavidin coated SPA beads.

After 15 minutes of incubation at room temperature to allow the biotinylated MCM2-streptavidin SPA beads interaction to occur, beads were trapped in a 96 wells filter plate (Unfilter^{R} GF/B™) using a Packard Cell Harvester (Filtermate), washed with distilled water and then counted using a Top Count (Packard).

Counts were blank-subtracted and then the experimental data (each point in triplicate) were analyzed for IC50 determination using a non-linear regression analysis (Sigma Plot).

Inhibition Assay of Aurora-2 Activity

The inhibiting activity and the potency of selected compounds was determined through a method of assay based on the use of the streptavidin scintillation proximity assay beads (amershampharmacia biotech) run in a 96 well plates. At the end of the reaction, the biotinylated peptide substrate was captured with the beads and subsequently allowed to stratify using CsCl₂.

When a radioactivity labeled phosphate moiety was transferred by the kinase to the beads-bound peptide, light emitted was measured in a scintillation counter.

The inhibition assay of Aurora-2 activity was performed in 96 wells plate according to the following protocol.

Kinase reaction: 8 μM biotinylated peptide (4 repeats of LRRWSLG), 10 MM ATP (0.5 uCi P³³ g-ATP), 10 nM Aurora2, 10 inhibitor in a final volume of 60μ buffer (HEPES 50 mM pH 7.0, MgCl₂ 10 mM, 1 mM DTT, 0.125 mg/ml BSA, 3 μM orthovanadate) were added to each well of a 96 U bottom well plate. After 30 minutes at room temperature incubation, reaction was stopped and biotinylated peptide captured by adding 100 μl of bead suspension.

Stratification: 100 μl of CsCl2 7.5 M were added to each well and let stand one hour before radioactivity was counted in the Top-Count instrument.

Results: data were analyzed and expressed as % inhibition referred to total activity of enzyme (=1100%).

All compounds showing inhibition ≧60% were further analyzed in order to study the potency of the inhibitor through IC50 calculation.

The protocol used was the same described above, except that serial dilution of the inhibitor was used. Experimental data were fitted by nonlinear regression using the following equation:

$$v = {v}_{0} + \frac{( {v}_{0} - {v}_{b} )}{1 + {10}^{n ( log {IC}_{50} - log [ I ] )}}$$

With v_{b} as the baseline velocity, v as the observed reaction velocity, v₀ as the velocity in the absence of inhibitors, and [I] as the inhibitor concentration.

The compounds of formula (I) of the present invention, suitable for administration to a mammal, e.g. to humans, can be administered by the usual routes and the dosage level depends upon the age, weight, conditions of the patient and the administration route.

For example, a suitable dosage adopted for oral administration of a compound of formula (I) may range from about 10 to about 500 mg pro dose, from 1 to 5 times daily.

The compounds of the invention can be administered in a variety of dosage forms, e.g. orally, in the form of tablets, capsules, sugar or film coated tablets, liquid solutions or suspensions; rectally in the form of suppositories; parenterally, e.g. intramuscularly, or by intravenous and/or intrathecal and/or intraspinal injection or infusion.

In addition, the compounds of the invention can be administered either as single agents or, alternatively, in combination with known anticancer treatments such as radiation therapy or chemotherapy regimen in combination with cytostatic or cytotoxic agents, antibiotic-type agents, alkylating agents, antimetabolite agents, hormonal agents, immunological agents, interferon-type agents, cyclooxygenase inhibitors (e.g. COX-2 inhibitors), metallomatrixprotease inhibitors, telomerase inhibitors, tyrosine kinase inhibitors, anti-growth factor receptor agents, anti-HER agents, anti-EGFR agents, anti-angiogenesis agents, farnesyl transferase inhibitors, ras-raf signal transduction pathway inhibitors, cell cycle inhibitors, other cdks inhibitors, tubulin binding agents, topoisomerase I inhibitors, topoisomerase R1 inhibitors, and the like.

As an example, the compounds of the invention can be administered in combination with one or more chemotherapeutic agents such as, for instance, exemestane, formestane, anastrozole, letrozole, fadrozole, taxane, taxane derivatives, encapsulated taxanes, CPT-11, camptothecin derivatives, anthracycline glycosides, e.g., doxorubicin, idarubicin, epirubicin, etoposide, navelbine, vinblastine, carboplatin, cisplatin, estramustine, celecoxib, tamoxifen, raloxifen, Sugen SU-5416, Sugen SU-6668, Herceptin, and the like, optionally within liposomal formulations thereof.

If formulated as a fixed dose, such combination products employ the compounds of this invention within the dosage range described above and the other pharmaceutically active agent within the approved dosage range.

Compounds of formula (I) may be used sequentially with known anticancer agents when a combination formulation is inappropriate.

It is therefore a further object of the invention a product or kit comprising the compound of formula (I) of the invention and one or more chemotherapeutic agents for simultaneous, separate or sequential use in anticancer therapy or for the treatment of cell proliferative disorders.

The present invention also includes pharmaceutical compositions comprising an effective amount of a compound of formula (I) or a pharmaceutically acceptable salt thereof in association with a pharmaceutically acceptable excipient, carrier or diluent.

The pharmaceutical compositions containing the compounds of the invention are usually prepared following conventional methods and are administered in a pharmaceutically suitable form.

For example, the solid oral forms may contain, together with the active compound, diluents, e.g. lactose, dextrose, saccharose, sucrose, cellulose, corn starch or potato starch; lubricants, e.g. silica, talc, stearic, magnesium or calcium stearate, and/or polyethylene glycols; binding agents, e.g. starches, arabic gum, gelatine, methylcellulose, carboxymethylcellulose or polyvinyl pyrrolidone; disaggregating agents, e.g. a starch, alginic, alginates or sodium starch glycolate; effervescing mixtures; dyestuffs; sweeteners; wetting agents such as lecithin, polysorbates, laurylsulphates; and, in general, non-toxic and pharmacologically inactive substances used in pharmaceutical formulations. Said pharmaceutical preparations may be manufactured in known manner, for example, by means of mixing, granulating, tabletting, sugar-coating, or film-coating processes.

The liquid dispersions for oral administration may be e.g. syrups, emulsions and suspensions.

The syrups may contain as carrier, for example, saccharose or saccharose with glycerine and/or mannitol and/or sorbitol.

The suspensions and the emulsions may contain as carrier, for example, a natural gum, agar, sodium alginate, pectin, methylcellulose, carboxymethylcellulose, or polyvinyl alcohol.

The suspension or solutions for intramuscular injections may contain, together with the active compound, a pharmaceutically acceptable carrier, e.g. sterile water, olive oil, ethyl oleate, glycols, e.g. propylene glycol, and, if desired, a suitable amount of lidocaine hydrochloride. The solutions for intravenous injections or infusions may contain as carrier, for example, sterile water or preferably they may be in the form of sterile, aqueous, isotonic saline solutions or they may contain as a carrier propylene glycol.

The suppositories may contain together with the active compound a pharmaceutically acceptable carrier, e.g. cocoa butter, polyethylene glycol, a polyoxyethylene sorbitan fatty ester surfactant or lecithin.

### EXAMPLES

The following examples are herewith intended to better illustrate the present invention without posing any limitation to it.

General Methods

Flash chromatografy was performed on silica gel (Merck grade 9385, 60A). SPE (Solid Phase Extraction) was performed using prepacked cartridges with silica gel available from several vendors. FT-IR was obtained using a Perkin-Elmer spectrophotometer with the micropellets. HPLC/MS was performed on a Waters X Terra RP 18 (4.6×50 mm, 3.5 μm) column using a Waters 2790 HPLC system equipped with a 996 Waters PDA detector and a Micromass mod. ZQ single quadrupole mass spectrometer, equipped with an electrospray (ESI) ion source. Mobile phase A was ammonium acetate 5 mM buffer (pH 5.5 with acetic acid/acetonitrile 95:5), and Mobile phase B was H₂O/acetonitrile (5:95). Gradient from 10 to 90% B in 8 minutes, hold 90% B 2 min. UV detection at 220 nm and 254 nm. Flow rate 1 ml/min. Injection volume 10 μl. Full scan, mass range from 100 to 800 amu. Capillary voltage was 2.5 KV; Source temp. was 120° C.; Cone was 10 V. Retention Times (HPLC r.t.) are given in minutes at 220 nm or 254 nm. Mass are given as m/z ratio.

When necessary compounds have been purified by Preparative HPLC on a Waters Symmetry C18 (19×50 mm, 5 um) column using a Waters preparative HPLC 600 equipped with a 996 Waters PDA detector and a Micromass mod. ZMD single quadrupole mass spectrometer, electrospray ionisation, positive mode. Mobile phase A was water 0.01% TFA, and Mobile phase B was acetonitrile. Gradient from 10 to 90%B in 8 min, hold 90%B 2 min. Flow rate 20 ml/m.

¹H-NMR spectroscopy was performed on a Mercury VX 400 operating at 400.45 MHz equipped with a 5 mm double resonance probe (1H {15N-31P} ID_PFG Varian).

### Example 1

1-[4-Amino-2-(4-benzyloxy-benzyloxy)-phenyl]-ethanone

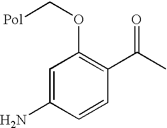

To 0.83 mmol of resin 2 (1 g) in 15 ml of DMA were added 6.64 mmol of 4-amino-2-hydroxyacetophenone (8 eq 1.0 g), 4.15 mmol of cesium carbonate (5 eq 1.35 g), and 0.83 mmol of tetrabutylammonium iodide (1 eq 0.3 g). The slurry was left under magnetic stirring for 24 h at room temperature, then the resin was filtered, washed with DMF (2×), MeOH, THF, MeOH, DCM (3×) and dried in vacuo to give 1.1 g of resin.

FT-IR KBr micropellet □ cm⁻¹: 1675 (C═O, s, st).

### Example 2

N-[4-Acetyl-3-(4-benzyloxy-benzyloxy-polystyrene)-phenyl]-4-methoxy-benzamide

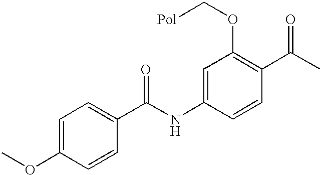

1-[4-Amino-2-(4-benzyloxy-benzyloxy)-phenyl]-ethanone (0.166 mmol, 0.2 g) was suspended with 0.5 ml of DiPEA (excess), 10 mg of DMAP in 5 ml of DCM. To the slurry was added under argon, 0.49 mmol of p-anisoyl chloride (3 eq, 85 mg). The mixture was stirred at room temperature for 18 h. The slurry was filtered and the resin washed with DMF, MeOH, DMF, MeOH, THF, MeOH, DCM (3×). The resin was dried under vacuum to give 0.22 g of product.

FT-IR KBr micropellet □ cm⁻¹: 1679 (C═O, s, b, st).

### Example 3

N-[4-Acetyl-3-(4-benzyloxy-benzyloxy-polystyrene)-phenyl]-4-methoxy-benzamide

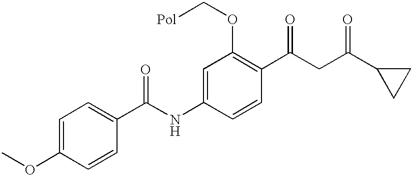

N-[4-Acetyl-3-(4-benzyloxy-benzyloxy-polystyrone)-phenyl]-4-methoxy-benzamide,

(0.166 mmol, 0.2 g) was suspended in a solution of 3.32 mmol of ethyl cyclopropyl carboxylate (20 eq, 568 mg) in 5 ml of DMA. To the slurry was added under argon, 4.98 mmol of sodium hydride (30 eq, 199 mg as 60% dispersion in paraffin oil). The mixture was heated under stirring at 90° for 1 h. The slurry was filtered and the resin washed with DMF, AcOH 20%, H₂O, DMF, MeOH, THF, MeOH, DCM (3×).

The resin was dried under vacuum and 0.22 g was obtained.

FT-IR KBr micropellet □cm⁻¹: 1603 (C═O, s, b, st).

### Example 4

N-[3-(4-Benzyloxy-benzyloxypolystyrene)-4-(3-cyclopropyl-3-oxo-propionyl)-phenyl]-4-methoxy-benzamide

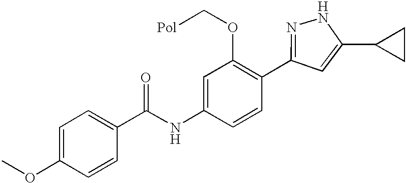

N-[3-(4-Benzyloxy-benzyloxypolystyrene)-4-(3-cyclopropyl-3-oxo-propionyl)-phenyl]-4-methoxy-benzamide (0.166 mmol, 0.2 g) was swelled in a solution of 16 mmol of hydrazine hydrate (100 eq), in 3 ml of DMA. The suspension was heated at 80° under magnetic stirring overnight. The resin was filtered and washed with DMF (2×), MeOH, THF, MeOH, DCM (3×) consecutively.

The resin was dried under vacuum and 0.21 g was obtained.

FT-IR KBr micropellet □ cm⁻¹: 3442 (N—H, b, st).

As further analytical investigation an aliquot of resin was incubated with TFA/DCM 50% for one hour, then filtered off and the solution was evaporated under nitrogen to dryness.

We obtained from 5 mg of resin (loading 0.83 mMol/g) 0.76 mg of N-[4-(5-Cyclopropyl-1H-pyrazol-3-yl)-3-hydroxy-phenyl]-4-methoxy-benzamide. The final product was obtained in 80% yield with 95% HPLC purity (according to HPLC analysis at 220 nm). [M+H]+=350 [M]−=348, HPLC r.t. 5.35. ¹H-NMR (DMSO-d₆), (ppm): 10.06 (s, 1H), 9.79 (s, 1H), 7.94 (d, 2H), 7.73 (d, 1H), 7.55 (s, 1H), 7.26 (d, 1H), 7.04 (d, 2H), 6.60 (s, 1H), 3.83 (s, 3H), 2.82 (m, 1H), 1.02 (m, 2H), 0.74 (m, 2H).

### Example 5

3-[2-(4-Benzyloxy-benzyloxypolystyrene)-4-(4-methoxy-benzoylamino)-phenyl]-5-cyclopropyl-pyrazole-1-carboxylic Acid Butylamide

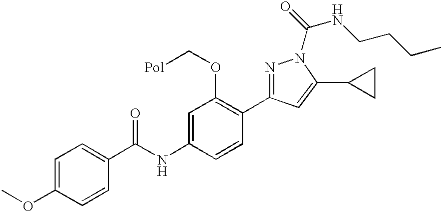

N-[3-(4-Benzyloxy-benzyloxypolystyrene)-4-(5-cyclopropyl-1H-pyrazol-3-yl)-phenyl]-4-methoxy-benzamide (0.124 mmol 0.15 g) was suspended in a solution of 0.124 mmol of DiPEA (1 eq, 22 μL) in 3 ml of toluene and to the suspension was added 1.2 mmol of n-butylisocyanate (10 eq, 100 μL) and stirred at 60° overnight. The resin was filtered and washed with DMF (2×), MeOH, THF, MeOH, DCM (3×) and dried in vacuo, to obtain 0.124 mg of resin.

FT-IR KBr micropellet □ cm⁻¹: 3442 (N-H, b, st,),1745 (C═O uerea, s, st) 1679 (C═O amide, s, st,).

### Example 6

5-Cyclopropyl-3-[2-hydroxy-4-(4-methoxy-benzoylamino)-phenyl]-pyrazole-1-carboxylic Acid Butylamide

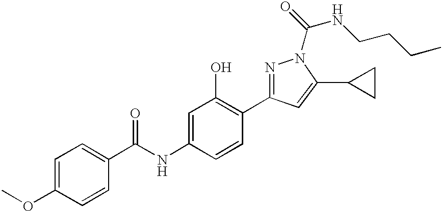

0.124 mmol, of 3-[2-(4-Benzyloxy-benzyloxypolystyrene)-4-(4-methoxy-benzoylamino)-phenyl]-5-cyclopropyl-pyrazole-1-carboxylic acid butylamide resin were swelled in 2 ml of 50% TFA in DCM. The resulting suspension was gently stirred or shaken at 22° C. for 1 h. The resin was filtered and washed with DCM, the solution was collected in a test tube and evaporated to dryness under nitrogen.

We obtained 20 mg (yield 40%) of product. LC/MS analysis showed 50% purity (254 nm. After filtration through 250 mg of silica gel packed in a 3 ml cartridge was recovered 11.5 mg.

LC/MS analysis gave 90% of purity HPLC r.t. 8.24 [M+H]+=449, [M−H]−=447.

¹H-NMR (DMSO-d₆), (ppm): 10.06 (s, 1H), 9.79 (s, 1H), 8.56 (t, 1H), 7.94 (d, 2H), 7.73 (d, 1H), 7.55 (s, 1H), 7.26 (d, 1H), 7.04 (d, 2H), 6.60 (s, 1H), 3.83 (s, 3H), 3.25 (m, 2H), 2.82 (m, 1H), 1.54 (m, 2H), 1.33 (m, 2H), 1.02 (m, 2H), 0.91 (t, 3H), 0.74 (m, 2H).

By working in an analogous way and by using the appropriate 2-hydroxyacetophenones derivatives, the appropriate carboxylic ester derivatives and the appropriate isocyanates derivatives, the following compounds were prepared:

N-ethyl-3-(2-hydroxyphenyl)-5-pyridin-4-yl-1H-pyrazole-1-carboxamide,

[M+H]+=309. ¹H-NMR (DMSO d₆) diagnostic signals (ppm): 8.73 (d, 2H), 7.20 (dd, 1H), 6.92 (d, 1H), 6.91 (dd, 1H), 2.81 (q, 2H), 1.12 (t, 3H).

5-Cyclopropyl-3-(2-hydroxyphenyl)-N-propyl-1H-pyrazole-1-carboxamide,

[M+H]+=285. ¹H-NMR (DMSO d₆) diagnostic signals (ppm): 9.78 (s, 1H), 8.67 (t, 1H), 7.78 (d, 1H), 7.21 (m, 1H), 6.84 (m, 2H), 6.63 (s, 1H), 3.22 (m, 2H), 2.75 (m, 1H), 1.58 (m, 1H), 1.01 (m, 2H), 0.89 (t, 3H), 0.74 (m, 2H).

Ethyl N-{[5-cyclopropyl-3-(2-hydroxyphenyl)-1H-pyrazol-1-yl]carbonyl}glycinate

[M+H]+=330. ¹H-NMR (DMSO d₆) diagnostic signals (ppm): 9.78 (s, 1H), 8.95 (t, 1H) 7.8 (d, 1H), 7.22 (dd, 1H), 6.93 (m, 2H), 6.68 (s, 1H), 4.15 (q, 2H), 4.01 (d, 2H), 2.73 (m, 1H), 1.21 (t, 3H), 1.01 (m, 2H), 0.76 (m, 2H).

N-allyl-5-cyclopropyl-3-(2-hydroxyphenyl)-1H-pyrazole-1-carboxamide,

[M+H]+=284. ¹H-NMR (DMSO d6) diagnostic signals (ppm): 9.85 (s, 1H), 8.82 (t, 1H), 7.81 (d, 1H), 7.2 (dd, 1H), 6.92 (m, 2H), 6.65 (s, 1H), 5.92 (m, 1H) 5.2 (d, 1H), 5.12 (d, 1H), 3.88 (dd, 2H), 2.75 (m, 1H), 1.02 (m, 2H), 0.75 (m, 2H).

N-cyclohexyl-5-cyclopropyl-3-(2-hydroxyphenyl)-1H-pyrazole-1-carboxamide

[M+H]+=326. ¹H-NMR (DMSO d₆) diagnostic signals (ppm): 9.88 (s, 1H), 8.23 (d, 1H), 7.79 (d, 1H), 7.2 (dd, 1H), 6.91 (d, 1H), 6.87 (dd, 1H), 6.62 (s, 1H), 3.62 (m, 1H), 2.75 (m, 1H), 1.82-1.15 (m, 10H), 1.02 (m, 2H), 0.75 (m, 2H).

5-Cyclopropyl-N-ethyl-3-(2-hydroxy-4-{[3-(trifluoromethyl)benzoyl]amino}phenyl)-1H-pyrazole-1-carboxamide,

[M+H]+=459. ¹H-NMR (DMSO d₆) diagnostic signals (ppm): 10.41 (s, 1H), 9.82 (s, 1H), 8.57 (t, 1H), 8.25 (s, 1H), 8.22 (d, 1H), 7.95 (d, 1H), 7.80 (d, 1H), 7.57 (s, 1H), 7.27 (d, 1H), 6.62 (s, 1H), 3.28 (m, 2H), 2.75 (m, 1H), 1.16 (t, 3H), 1.02 (m, 2H), 0.75 (m, 2H).

Ethyl-N-{[5-[2-(dimethylamino)ethyl]-3-(2-hydroxyphenyl)-1H-pyrazol-1-yl]carbonyl}glycinate,

[M+H]+=361 ¹H-NMR (DMSO d₆) diagnostic signals (ppm): 9.02 (s, 1H), 9.05 (t, 1H), 7.90 (d, 1H), 7.22 (dd, 1H), 6.97 (s, 1H), 6.95 (d, 1H), 6.9 (dd, 1H), 4.13 (q, 2H), 4.02 (d, 2H), 3.41 (bs, 4H), 2.83 (s, 6H), 1.21 (t, 3H).

5-Cyclopropyl-N-ethyl-3-(2-hydroxy-4-methoxyphenyl)-1H-pyrazole-1-carboxamide,

[M+H]+=302. ¹H-NMR (DMSO d₆) diagnostic signals (ppm): 9.91 (s, 1H), 8.55 (t, 1H), 7.67 (d, 1H), 6.56 (s, 1H), 6.46 (m, 2H), 3.74 (s, 3H), 3.22 (m, 2H), 2.75 (m, 1H), 1.15 (t, 3H), 1.02 (m, 2H), 0.75 (m, 2H).

Ethyl-N-{[5-cyclopropyl-3-(2-hydroxy-4-methoxyphenyl)-1H-pyrazol-1-yl]carbonyl}glycinate,

[M+H]+=360. ¹H-NMR (DMSO d₆) diagnostic signals (ppm): 9.89 (s, 1H), 8.94 (t, 1H), 7.66 (d, 1H), 6.61 (s, 1H), 6.48 (m, 2H), 4.13 (q, 2H), 4.02 (d, 2H), 3.74 (s, 3H), 2.75 (m, 1H), 1.21 (t, 3H), 1.02 (m, 2H), 0.75 (m, 2H).

5-Cyelopropyl-N-ethyl-3-(2-hydroxy-5-methoxyphenyl)-1H-pyrazole-1-earboxamide,

[M+H]+=302. ¹H-NMR (DMSO d₆) diagnostic signals (ppm): 9.33 (s, 1H), 8.58 (t, 1H), 7.34 (d, 1H), 6.82 (m, 2H), 6.68 (s, 1H), 3.72 (s, 3H), 3.22 (m, 2H), 2.74 (m, 1H), 1.15 (t, 3H), 1.02 (m, 2H), 0.75 (m, 2H).

5-Cyclopropyl-N-ethyl-3-(2-hydroxy-5-methylphenyl)-1H-pyrazole-1-carboxamide,

[M+H]+=286. ¹H-NMR (DMSO d₆) diagnostic signals (ppm): 9.33 (s, 1H), 8.57 (t, 1H), 7.34 (d, 1H), 6.81 (m, 2H), 6.68 (s, 1H), 3.22 (m, 2H), 2.74 (m, 1H), 2.22(s, 3H), 1.15 (t, 3H), 1.02 (m, 2H), 0.75 (m, 2H).

Ethyl-N-{[5-cyclopropyl-3-(2-hydroxy-5-methylphenyl)-1H-pyrazol-1-yl]carbonyl}glycinate,

[M+H]+=344. ¹H-NMR (DMSO d₆) diagnostic signals (ppm): 9.52 (s, 1H), 8.58 (t, 1H), 7.59 (d, 1H), 7.02 (d, 1H), 6.81 (d, 1H), 6.62 (s, 1H), 3.22 (m, 2H), 2.75 (m, 1H), 2.23 (s, 3H), 1.15 (t, 3H), 1.02 (m, 2H), 0.75 (m, 2H).

N-butyl-5-cyclopropyl-3-(2-hydroxy-4-methoxyphenyl)-1H-pyrazole-1-carboxamide,

[M+H]+=330. ¹H-NMR (DMSO d₆) diagnostic signals (ppm): 9.82 (s, 1H), 8.57 (t, 1H), 7.67 (d, 1H), 6.56 (s, 1H), 6.47 (m, 2H), 3.74 (s, 3H), 3.25 (m, 2H), 2.75 (m, 1H), 1.55 (m, 2H), 1.33 (m, 2H), 1.02 (m, 2H), 0.90 (t, 3H), 0.75 (m, 2H).

N-butyl-S-cyclopropyl-3-(2-hydroxy-5-methylphenyl)-1H-pyrazole-1-carboxamide,

[M+H]+=314. ¹H-NNMR (DMSO d₆) diagnostic signals (ppm): 9.55 (s, 1H), 8.58 (t, 1H), 7.58 (d, 1H), 7.0 (d, 1H), 6.81 (d, 1H), 6.63 (s, 1H), 3.25 (m, 2H), 2.75 (m, 1H), 2.23 (s, 3H),1.56 (m, 2H), 1.34 (m, 2H), 1.01 (m, 2H), 0.91 (t, 3H), 0.75 (m, 2H).

N-(3-{1-[(butylamino)carbonyl]-5-cyclopropyl-1H-pyrazol-3-yl}-4-hydroxyphenyl)isonicotinamide,

[M+H]+=420. ¹H-NMR (DMSO d₆) diagnostic signals (ppm): 9.68 (s, 1H), 8.78 (d, 1H), 8.52 (t, 1H), 8.02 (s, 1H), 7.86 (d, 1H), 7.55 (dd, 1H), 6.94 (d, 1H), 6.51 (s, 1H), 3.25 (m, 2H), 2.75 (m, 1H), 1.56 (m, 2H), 1.34 (m, 2H), 1.01 (m, 2H), 0.87 (t, 3H), 0.75 (m, 2H).

N-(4-{1-[(butylamino)carbonyl]-5-cyclopropyl-1H-pyrazol-3-yl}-3-hydroxyphenyl)isonicotinamide,

[M+H]+=420. ¹H-NMR (DMSO d₆) diagnostic signals (ppm): 10.48 (s, 1H), 9.87 (s, 1H), 8.78 (d, 1H), 8.55 (t, 1H), 7.86 (d, 2H), 7.78 (d, 1H), 7.55 (s, 1H), 7.27 (dd, 1H), 6.61 (s, 1H), 3.23 (m, 2H), 2.76 (m, 1H), 1.56 (m, 2H), 1.34 (m, 2H), 1.01 (m, 2H), 0.91 (t, 3H), 0.75 (m, 2H).

3-{4-[(3-Benzodioxol-5-ylcarbonyl)amino]-2-hydroxypheny}-N-butyl-5-cyclobutyl-1H-pyrazole-1-carboxamide,

[M+H]+=477. ¹H-NMR (DMSO d₆) diagnostic signals ppm): 10.0 (s, 1H), 8.52 (t, 1H), 7.61 (d, 1H), 7.59 9d, 1H), 7.56 (d, 1H), 7.45 (d, 1H), 7.27 (d, 1H), 7.05 (d, 1H) 6.65 (s, 1H), 6.11 (s, 2H), 3.58 (m, 1H), 3.23 (m, 2H), 2.31 (m, 2H), 2.19 (m, 2H), 1.97 (m, 2H), 1.56 (m, 2H), 1.34 (m, 2H), 0.91 (t, 3H).

3-{4-[(1,3-Benzodioxol-5-ylcarbonyl)amino]-2-hydroxyphenyl}-N-butyl-5-cyclopentyl-1H-pyrazole-1-carboxamide,

[M+H]+=491. ¹H-NMR (DMSO d₆) diagnostic signals (ppm): 10.05 (s, 1H), 8.52 (t, 1H), 7.60 (d, 1H), 7.58 (d, 1H), 7.48 (d, 1H), 7.42, (s, 1H), 7.04 (d, 1H), 6.58 (s, 1H), 6.11 (s, 2H), 3.23 (m, 2H), 3.11 (m, 1H), 2.1 (m, 2H), 1.75 (m, 2H), 1.6 (m, 4H), 1.56 (m, 2H), 1.34 (m, 2H), 0.91 (t, 3H).

N-Allyl-5-cyclopropyl-3-(4-fluoro-2-hydroxyphenyl)-1H-pyrazole-1-carboxamide,

[M+H]+=302. HPLC r.t.=6.21-5-(3,4-Dimethoxyphenyl)-N-ethyl-3-(2-hydroxy-5-methoxyphenyl)-1H-pyrazole-1-carboxamide,

[M+H]+=398. HPLC r.t.=4.59

N-Allyl-5-[2-(dimethylamino)ethyl]-3-(2-hydroxy-5-methylphenyl)-1H-pyrazole-1-carboxamide,

[M+H]+=329 HPLC r.t.=3.75.

5-[2-(Dimethylamino)ethyl]-3-(2-hydroxy-5-methoxyphenyl)-N-(2-phenylethyl)-1H-pyrazole-1-carboxamide,

[M+H]+=409. HPLC r.t.=4.39.

Ethyl-N-{[5-(3,4-dimethoxyphenyl)-3-(2-hydroxy-5-methoxyphenyl)-1H-pyrazol-1-yl]carbonyl}glycinate,

[M+H]+=456. HPLC r.t.=4.69

5-Cyclopropyl-N-ethyl-3-(4-fluoro-2-hydroxyphenyl)-1H-pyrazole-1-carboxamide,

[M+H]+=290. HPLC r.t.=6.01

Ethyl-N-{[3-(5-chloro-2-hydroxyphenyl)-5-cyclopropyl-1H-pyrazol-1-yl]carbonyl}glycinate,

[M+H]+=364. HPLC r.t.=6.21

5-Cyclopropyl-3-(2-hydroxy-5-methoxyphenyl)-N-(2-phenylethyl)-1H-pyrazole-1-carboxamide,

[M+H]+=377. HPLC r.t.=6.55.

5-Cyclopropyl-N-ethyl-3-(2-hydroxyphenyl)-1H-pyrazole-1-carboxamide,

[M+H]+=271. HPLC r.t.=5.76.

N-allyl-5-(3,4-dimethoxyphenyl)-3-(2-hydroxy-5-methoxyphenyl)-1H-pyrazole-1-carboxamide,

[M+H]+=410. HPLC r.t.=4.71.

N-benzyl-5-(3,4-dimethoxyphenyl)-3-(2-hydroxy-5-methylphenyl)-1H-pyrazole-1-carboxamide,

[M+H]+=444. HPLC r.t.=5.41.

N-allyl-5-cyclopropyl-3-(2-hydroxy-5-methoxyphenyl)-1H-pyrazole-1-carboxamide,

[M+H]+=314. HPLC r.t.=5.80.

N-benzyl-5-[2-(dimethylamino)ethyl]-3-(2-hydroxy-5-methoxyphenyl)-1H-pyrazole-1-1-carboxamide,

[M+H]+=394. HPLC r.t. 4.20.

5-Cyclopropyl-3-(2-hydroxy-5-methylphenyl)-N-(2-phenylethyl)-1H-pyrazole-1-carboxamide,

[M+H]+=362. HPLC r.t.=6.90.

5-[2-(Dimethylamino)ethyl]-N-ethyl-3-(2-hydroxyphenyl)-1H-pyrazole-1-carboxamide,

[M+H]+=302. HPLC r.t.=3.24.

Ethyl N-[(5-cyclobutyl-3-{2-hydroxy-4-[(3-methoxybenzoyl)amino]phenyl}-1H-pyrazol-1-yl)carbonyl]glycinate,

[M+H]+=493. HPLC r.t.=7.27.

Ethyl N-[(5-cyclopropyl-3-{2-hydroxy-4-[(3-methoxybenzoyl)amino]phenyl}-1H-pyrazol-1-yl)carbonyl]glycinate,

[M+H]+=479. HPLC r.t.=6.87.

Operting in an analogous way, the following compounds are also obtained:

| | |
|---|---|
| 1 | 3-{4-[(2-chlorobenzoyl)amino]-2-hydroxyphenyl}-5-cyclopropyl-N-isopropyl-1H- |
| | pyrazole-1-carboxamide |
| 2 | 3-{4-[(2-chlorobenzoyl)amino]-2-hydroxyphenyl}-5-cyclopropyl-N-ethyl-1H- |
| | pyrazole-1-carboxamide |
| 3 | N-allyl-3-{4-[(2-chlorobenzoyl)amino]-2-hydroxyphenyl}-5-cyclopropyl-1H- |
| | pyrazole-1-carboxamide |
| 4 | 3-{4-[(2-chlorobenzoyl)amino]-2-hydroxyphenyl}-5-cyclopropyl-N-propyl-1H- |
| | pyrazole-1-carboxamide |
| 5 | N-benzyl-3-{4-[(2-chlorobenzoyl)amino]-2-hydroxyphenyl}-5-cyclopropyl-1H- |
| | pyrazole-1-carboxamide |
| 6 | 3-{4-[(2-chlorobenzoyl)amino]-2-hydroxyphenyl}-5-cyclopropyl-N-(2- |
| | phenylethyl)-1H-pyrazole-1-carboxamide |
| 7 | 3-{4-[(2-chlorobenzoyl)amino]-2-hydroxyphenyl}-5-cyclopropyl-N-(2- |
| | methylbenzyl)-1H-pyrazole-1-carboxamide |
| 8 | 3-{4-[(2-chlorobenzoyl)amino]-2-hydroxyphenyl}-5-cyclopropyl-N-(3- |
| | methylbenzyl)-1H-pyrazole-1-carboxamide |
| 9 | 3-{4-[(2-chlorobenzoyl)amino]-2-hydroxyphenyl}-5-cyclopropyl-N-(4- |
| | methylbenzyl)-1H-pyrazole-1-carboxamide |
| 10 | 3-{4-[(2-chlorobenzoyl)amino]-2-hydroxyphenyl}-N-(2-chlorobenzyl)-5- |
| | cyclopropyl-1H-pyrazole-1-carboxamide |
| 11 | 3-{4-[(2-chlorobenzoyl)amino]-2-hydroxyphenyl}-5-cyclopropyl-N-(4- |
| | fluorobenzyl)-1H-pyrazole-1-carboxamide |
| 12 | 3-{4-[(2-chlorobenzoyl)amino]-2-hydroxyphenyl}-5-cyclopropyl-N-(3,4- |
| | dichlorobenzyl)-1H-pyrazole-1-carboxamide |
| 13 | 3-{4-[(2-chlorobenzoyl)amino]-2-hydroxyphenyl}-5-cyclopropyl-N-(4- |
| | methoxybenzyl)-1H-pyrazole-1-carboxamide |
| 14 | N-(sec-butyl)-3-{4-[(2-chlorobenzoyl)amino]-2-hydroxyphenyl}-5-cyclopropyl- |
| | 1H-pyrazole-1-carboxamide |
| 15 | 3-{4-[(2-chlorobenzoyl)amino]-2-hydroxyphenyl}-5-cyclopropyl-N-(2- |
| | fluorobenzyl)-1H-pyrazole-1-carboxamide |
| 16 | 3-{4-[(2-chlorobenzoyl)amino]-2-hydroxyphenyl}-5-cyclopropyl-N-(3- |
| | fluorobenzyl)-1H-pyrazole-1-carboxamide |
| 17 | 3-{4-[(2-chlorobenzoyl)amino]-2-hydroxyphenyl}-N,5-dicyclopropyl-1H- |
| | pyrazole-1-carboxamide |
| 18 | 3-{4-[(2-chlorobenzoyl)amino]-2-hydroxyphenyl}-5-cyclobutyl-N-isopropyl-1H- |
| | pyrazole-1-carboxamide |
| 19 | 3-{4-[(2-chlorobenzoyl)amino]-2-hydroxyphenyl}-5-cyclobutyl-N-ethyl-1H- |
| | pyrazole-1-carboxamide |
| 20 | N-allyl-3-{4-[(2-chlorobenzoyl)amino]-2-hydroxyphenyl}-5-cyclobutyl-1H- |
| | pyrazole-1-carboxamide |
| 21 | 3-{4-[(2-chlorobenzoyl)amino]-2-hydroxyphenyl}-5-cyclobutyl-N-propyl-1H- |
| | pyrazole-1-carboxamide |
| 22 | N-benzyl-3-{4-[(2-chlorobenzoyl)amino]-2-hydroxyphenyl}-5-cyclobutyl-1H- |
| | pyrazole-1-carboxamide |
| 23 | 3-{4-[(2-chlorobenzoyl)amino]-2-hydroxyphenyl}-5-cyclobutyl-N-(2- |
| | phenylethyl)-1H-pyrazole-1-carboxamide |
| 24 | 3-{4-[(2-chlorobenzoyl)amino]-2-hydroxyphenyl}-5-cyclobutyl-N-(2- |
| | methylbenzyl)-1H-pyrazole-1-carboxamide |
| 25 | 3-{4-[(2-chlorobenzoyl)amino]-2-hydroxyphenyl}-5-cyclobutyl-N-(3- |
| | methylbenzyl)-1H-pyrazole-1-carboxamide |
| 26 | 3-{4-[(2-chlorobenzoyl)amino]-2-hydroxyphenyl}-5-cyclobutyl-N-(4- |
| | methylbenzyl)-1H-pyrazole-1-carboxamide |
| 27 | 3-{4-[(2-chlorobenzoyl)amino]-2-hydroxyphenyl}-N-(2-chlorobenzyl)-5- |
| | cyclobutyl-1H-pyrazole-1-carboxamide |
| 28 | 3-{4-[(2-chlorobenzoyl)amino]-2-hydroxyphenyl}-5-cyclobutyl-N-(4- |
| | fluorobenzyl)-1H-pyrazole-1-carboxamide |
| 29 | 3-{4-[(2-chlorobenzoyl)amino]-2-hydroxyphenyl}-5-cyclobutyl-N-(3,4- |
| | dichlorobenzyl)-1H-pyrazole-1-carboxamide |
| 30 | 3-{4-[(2-chlorobenzoyl)amino]-2-hydroxyphenyl}-5-cyclobutyl-N-(4- |
| | methoxybenzyl)-1H-pyrazole-1-carboxamide |
| 31 | N-(sec-butyl)-3-{4-[(2-chlorobenzoyl)amino]-2-hydroxyphenyl}-5-cyclobutyl-1H- |
| | pyrazole-1-carboxamide |
| 32 | 3-{4-[(2-chlorobenzoyl)amino]-2-hydroxyphenyl}-5-cyclobutyl-N-(2- |
| | fluorobenzyl)-1H-pyrazole-1-carboxamide |
| 33 | 3-{4-[(2-chlorobenzoyl)amino]-2-hydroxyphenyl}-5-cyclobutyl-N-(3- |
| | fluorobenzyl)-1H-pyrazole-1-carboxamide |
| 34 | 3-{4-[(2-chlorobenzoyl)amino]-2-hydroxyphenyl}-5-cyclobutyl-N-cyclopropyl- |
| | 1H-pyrazole-1-carboxamide |
| 35 | 3-{4-[(2-chlorobenzoyl)amino]-2-hydroxyphenyl}-5-cyclopentyl-N-isopropyl-1H- |
| | pyrazole-1-carboxamide |
| 36 | 3-{4-[(2-chlorobenzoyl)amino]-2-hydroxyphenyl}-5-cyclopentyl-N-ethyl-1H- |
| | pyrazole-1-carboxamide |
| 37 | N-allyl-3-{4-[(2-chlorobenzoyl)amino]-2-hydroxyphenyl}-5-cyclopentyl-1H- |
| | pyrazole-1-carboxamide |
| 38 | 3-{4-[(2-chlorobenzoyl)amino]-2-hydroxyphenyl}-5-cyclopentyl-N-propyl-1H- |
| | pyrazole-1-carboxamide |
| 39 | N-benzyl-3-{4-[(2-chlorobenzoyl)amino]-2-hydroxyphenyl}-5-cyclopentyl-1H- |
| | pyrazole-1-carboxamide |
| 40 | 3-{4-[(2-chlorobenzoyl)amino]-2-hydroxyphenyl}-5-cyclopentyl-N-(2- |
| | phenylethyl)-1H-pyrazole-1-carboxamide |
| 41 | 3-{4-[(2-chlorobenzoyl)amino]-2-hydroxyphenyl}-5-cyclopentyl-N-(2- |
| | methylbenzyl)-1H-pyrazole-1-carboxamide |
| 42 | 3-{4-[(2-chlorobenzoyl)amino]-2-hydroxyphenyl}-5-cyclopentyl-N-(3- |
| | methylbenzyl)-1H-pyrazole-1-carboxamide |
| 43 | 3-{4-[(2-chlorobenzoyl)amino]-2-hydroxyphenyl}-5-cyclopentyl-N-(4- |
| | methylbenzyl)-1H-pyrazole-1-carboxamide |
| 44 | 3-{4-[(2-chlorobenzoyl)amino]-2-hydroxyphenyl}-N-(2-chlorobenzyl)-5- |
| | cyclopentyl-1H-pyrazole-1-carboxamide |
| 45 | 3-{4-[(2-chlorobenzoyl)amino]-2-hydroxyphenyl}-5-cyclopentyl-N-(4- |
| | fluorobenzyl)-1H-pyrazole-1-carboxamide |
| 46 | 5-cyclopropyl-3-{4-[(2,6-dichlorobenzoyl)amino]-2-hydroxyphenyl}-N-isopropyl- |
| | 1H-pyrazole-1-carboxamide |
| 47 | 3-{4-[(2-chlorobenzoyl)amino]-2-hydroxyphenyl}-5-cyclopentyl-N-(4- |
| | methoxybenzyl)-1H-pyrazole-1-carboxamide |
| 48 | N-(sec-butyl)-3-{4-[(2-chlorobenzoyl)amino]-2-hydroxyphenyl}-5-cyclopentyl- |
| | 1H-pyrazole-1-carboxamide |
| 49 | 3-{4-[(2-chlorobenzoyl)amino]-2-hydroxyphenyl}-5-cyclopentyl-N-(2- |
| | fluorobenzyl)-1H-pyrazole-1-carboxamide |
| 50 | 3-{4-[(2-chlorobenzoyl)amino]-2-hydroxyphenyl}-5-cyclopentyl-N-(3- |
| | fluorobenzyl)-1H-pyrazole-1-carboxamide |
| 51 | 3-{4-[(2-chlorobenzoyl)amino]-2-hydroxyphenyl}-5-cyclopentyl-N-cyclopropyl- |
| | 1H-pyrazole-1-carboxamide |
| 52 | 5-cyclopropyl-3-{4-[(2,6-dichlorobenzoyl)amino]-2-hydroxyphenyl}-N-isopropyl- |
| | 1H-pyrazole-1-carboxamide |
| 53 | 5-cyclopropyl-3-{4-[(2,6-dichlorobenzoyl)amino]-2-hydroxyphenyl}-N-ethyl-1H- |
| | pyrazole-1-carboxamide |
| 54 | N-allyl-5-cyclopropyl-3-{4-[(2,6-dichlorobenzoyl)amino]-2-hydroxyphenyl}-1H- |
| | pyrazole-1-carboxamide |
| 55 | 5-cyclopropyl-3-{4-[(2,6-dichlorobenzoyl)amino]-2-hydroxyphenyl}-N-propyl- |
| | 1H-pyrazole-1-carboxamide |
| 56 | N-benzyl-5-cyclopropyl-3-{4-[(2,6-dichlorobenzoyl)amino]-2-hydroxyphenyl}- |
| | 1H-pyrazole-1-carboxamide |
| 57 | 5-cyclopropyl-3-{4-[(2,6-dichlorobenzoyl)amino]-2-hydroxyphenyl}-N-(2- |
| | phenylethyl)-1H-pyrazole-1-carboxamide |
| 58 | 5-cyclopropyl-3-{4-[(2,6-dichlorobenzoyl)amino]-2-hydroxyphenyl}-N-(2- |
| | methylbenzyl)-1H-pyrazole-1-carboxamide |
| 59 | 5-cyclopropyl-3-{4-[(2,6-dichlorobenzoyl)amino]-2-hydroxyphenyl}-N-(3,4- |
| | dichlorobenzyl)-1H-pyrazole-1-carboxamide |
| 60 | 5-cyclopropyl-3-{4-[(2,6-dichlorobenzoyl)amino]-2-hydroxyphenyl}-N-(4- |
| | methylbenzyl)-1H-pyrazole-1-carboxamide |
| 61 | N-(2-chlorobenzyl)-5-cyclopropyl-3-{4-[(2,6-dichlorobenzoyl)amino]-2- |
| | hydroxyphenyl}-1H-pyrazole-1-carboxamide |
| 62 | 5-cyclopropyl-3-{4-[(2,6-dichlorobenzoyl)amino]-2-hydroxyphenyl}-N-(4- |
| | fluorobenzyl)-1H-pyrazole-1-carboxamide |
| 63 | 5-cyclopropyl-3-{4-[(2,6-dichlorobenzoyl)amino]-2-hydroxyphenyl}-N-(3,4- |
| | dichlorobenzyl)-1H-pyrazole-1-carboxamide |
| 64 | 5-cyclopropyl-3-{4-[(2,6-dichlorobenzoyl)amino]-2-hydroxyphenyl}-N-(4- |
| | methoxybenzyl)-1H-pyrazole-1-carboxamide |
| 65 | N-(sec-butyl)-5-cyclopropyl-3-{4-[(2,6-dichlorobenzoyl)amino]-2- |
| | hydroxyphenyl}-1H-pyrazole-1-carboxamide |
| 66 | 5-cyclopropyl-3-{4-[(2,6-dichlorobenzoyl)amino]-2-hydroxyphenyl}-N-(2- |
| | fluorobenzyl)-1H-pyrazole-1-carboxamide |
| 67 | 5-cyclopropyl-3-{4-[(2,6-dichlorobenzoyl)amino]-2-hydroxyphenyl}-N-(3- |
| | fluorobenzyl)-1H-pyrazole-1-carboxamide |
| 68 | N,5-dicyclopropyl-3-{4-[(2,6-dichlorobenzoyl)amino]-2-hydroxyphenyl}-1H- |
| | pyrazole-1-carboxamide |
| 69 | 5-cyclobutyl-3-{4-[(2,6-dichlorobenzoyl)amino]-2-hydroxyphenyl}-N-isopropyl- |
| | 1H-pyrazole-1-carboxamide |
| 70 | 5-cyclobutyl-3-{4-[(2,6-dichlorobenzoyl)amino]-2-hydroxyphenyl}-N-ethyl-1H- |
| | pyrazole-1-carboxamide |
| 71 | N-allyl-5-cyclobutyl-3-{4-[(2,6-dichlorobenzoyl)amino]-2-hydroxyphenyl}-1H- |
| | pyrazole-1-carboxamide |
| 72 | 5-cyclobutyl-3-{4-[(2,6-dichlorobenzoyl)amino]-2-hydroxyphenyl}-N-propyl-1H- |
| | pyrazole-1-carboxamide |
| 73 | N-benzyl-5-cyclobutyl-3-{4-[(2,6-dichlorobenzoyl)amino]-2-hydroxyphenyl}-1H- |
| | pyrazole-1-carboxamide |
| 74 | 5-cyclobutyl-3-{4-[(2,6-dichlorobenzoyl)amino]-2-hydroxyphenyl}-N-(2- |
| | phenylethyl)-1H-pyrazole-1-carboxamide |
| 75 | 5-cyclobutyl-3-{4-[(2,6-dichlorobenzoyl)amino]-2-hydroxyphenyl}-N-(2- |
| | methylbenzyl)-1H-pyrazole-1-carboxamide |
| 76 | 5-cyclobutyl-3-{4-[(2,6-dichlorobenzoyl)amino]-2-hydroxyphenyl}-N-(3- |
| | methylbenzyl)-1H-pyrazole-1-carboxamide |
| 77 | 5-cyclobutyl-3-{4-[(2,6-dichlorobenzoyl)amino]-2-hydroxyphenyl}-N-(4- |
| | methylbenzyl)-1H-pyrazole-1-carboxamide |
| 78 | N-(2-chlorobenzyl)-5-cyclobutyl-3-{4-[(2,6-dichlorobenzoyl)amino]-2- |
| | hydroxyphenyl}-1H-pyrazole-1-carboxamide |
| 79 | 5-cyclobutyl-3-{4-[(2,6-dichlorobenzoyl)amino]-2-hydroxyphenyl}-N-(4- |
| | fluorobenzyl)-1H-pyrazole-1-carboxamide |
| 80 | 5-cyclobutyl-3-{4-[(2,6-dichlorobenzoyl)amino]-2-hydroxyphenyl}-N-(3,4- |
| | dichlorobenzyl)-1H-pyrazole-1-carboxamide |
| 81 | 5-cyclobutyl-3-{4-[(2,6-dichlorobenzoyl)amino]-2-hydroxyphenyl}-N-(4- |
| | methoxybenzyl)-1H-pyrazole-1-carboxamide |
| 82 | N-(sec-butyl)-5-cyclobutyl-3-{4-[(2,6-dichlorobenzoyl)amino]-2-hydroxyphenyl}- |
| | 1H-pyrazole-1-carboxamide |
| 83 | 5-cyclobutyl-3-{4-[(2,6-dichlorobenzoyl)amino]-2-hydroxyphenyl}-N-(2- |
| | fluorobenzyl)-1H-pyrazole-1-carboxamide |
| 84 | 5-cyclobutyl-3-{4-[(2,6-dichlorobenzoyl)amino]-2-hydroxyphenyl}-N-(3- |
| | fluorobenzyl)-1H-pyrazole-1-carboxamide |
| 85 | 5-cyclobutyl-N-cyclopropyl-3-{4-[(2,6-dichlorobenzoyl)amino]-2- |
| | hydroxyphenyl}-1H-pyrazole-1-carboxamide |
| 86 | 5-cyclopentyl-3-{4-[(2,6-dichlorobenzoyl)amino]-2-hydroxyphenyl}-N-isopropyl- |
| | 1H-pyrazole-1-carboxamide |
| 87 | 5-cyclopentyl-3-{4-[(2,6-dichlorobenzoyl)amino]-2-hydroxyphenyl}-N-ethyl-1H- |
| | pyrazole-1-carboxamide |
| 88 | N-allyl-5-cyclopentyl-3-{4-[(2,6-dichlorobenzoyl)amino]-2-hydroxyphenyl}-1H- |
| | pyrazole-1-carboxamide |
| 89 | 5-cyclopentyl-3-{4-[(2,6-dichlorobenzoyl)amino]-2-hydroxyphenyl}-N-propyl- |
| | 1H-pyrazole-1-carboxamide |
| 90 | N-benzyl-5-cyclopentyl-3-{4-[(2,6-dichlorobenzoyl)amino]-2-hydroxyphenyl}- |
| | 1H-pyrazole-1-carboxamide |
| 91 | 5-cyclopentyl-3-{4-[(2,6-dichlorobenzoyl)amino]-2-hydroxyphenyl}-N-(2- |
| | phenylethyl)-1H-pyrazole-1-carboxamide |
| 92 | 5-cyclopentyl-3-{4-[(2,6-dichlorobenzoyl)amino]-2-hydroxyphenyl}-N-(2- |
| | methylbenzyl)-1H-pyrazole-1-carboxamide |
| 93 | 5-cyclopentyl-3-{4-[(2,6-dichlorobenzoyl)amino]-2-hydroxyphenyl}-N-(3- |
| | methylbenzyl)-1H-pyrazole-1-carboxamide |
| 94 | 5-cyclopentyl-3-{4-[(2,6-dichlorobenzoyl)amino]-2-hydroxyphenyl}-N-(4- |
| | methylbenzyl)-1H-pyrazole-1-carboxamide |
| 95 | N-(2-chlorobenzyl)-5-cyclopentyl-3-{4-[(2,6-dichlorobenzoyl)amino]-2- |
| | hydroxyphenyl}-1H-pyrazole-1-carboxamide |
| 96 | 5-cyclopentyl-3-{4-[(2,6-dichlorobenzoyl)amino]-2-hydroxyphenyl}-N-(4- |
| | fluorobenzyl)-1H-pyrazole-1-carboxamide |
| 97 | 5-cyclopentyl-3-{4-[(2,6-dichlorobenzoyl)amino]-2-hydroxyphenyl}-N-(3,4- |
| | dichlorobenzyl)-1H-pyrazole-1-carboxamide |
| 98 | 5-cyclopentyl-3-{4-[(2,6-dichlorobenzoyl)amino]-2-hydroxyphenyl}-N-(4- |
| | methoxybenzyl)-1H-pyrazole-1-carboxamide |
| 99 | N-(sec-butyl)-5-cyclopentyl-3-{4-[(2,6-dichlorobenzoyl)amino]-2- |
| | hydroxyphenyl}-1H-pyrazole-1-carboxamide |
| 100 | 5-cyclopentyl-3-{4-[(2,6-dichlorobenzoyl)amino]-2-hydroxyphenyl}-N-(2- |
| | fluorobenzyl)-1H-pyrazole-1-carboxamide |
| 101 | 5-cyclopentyl-3-{4-[(2,6-dichlorobenzoyl)amino]-2-hydroxyphenyl}-N-(3- |
| | fluorobenzyl)-1H-pyrazole-1-carboxamide |
| 102 | 5-cyclopentyl-N-cyclopropyl-3-{4-[(2,6-dichlorobenzoyl)amino]-2- |
| | hydroxyphenyl}-1H-pyrazole-1-carboxamide |
| 103 | 5-cyclopropyl-3-{2-hydroxy-4-[(2-methoxybenzoyl)amino]phenyl}-N-isopropyl- |
| | 1H-pyrazole-1-carboxamide |
| 104 | 5-cyclopropyl-N-ethyl-3-{2-hydroxy-4-[(2-methoxybenzoyl)amino]phenyl}-1H- |
| | pyrazole-1-carboxamide |
| 105 | N-allyl-5-cyclopropyl-3-{2-hydroxy-4-[(2-methoxybenzoyl)amino]phenyl}-1H- |
| | pyrazole-1-carboxamide |
| 106 | 5-cyclopropyl-3-{2-hydroxy-4-[(2-methoxybenzoyl)amino]phenyl}-N-propyl-1H- |
| | pyrazole-1-carboxamide |
| 107 | N-benzyl-5-cyclopropyl-3-{2-hydroxy-4-[(2-methoxybenzoyl)amino]phenyl}-1H- |
| | pyrazole-1-carboxamide |
| 108 | 5-cyclopropyl-3-{2-hydroxy-4-[(2-methoxybenzoyl)amino]phenyl}-N-(2- |
| | phenylethyl)-1H-pyrazole-1-carboxamide |
| 109 | 5-cyclopropyl-3-{2-hydroxy-4-[(2-methoxybenzoyl)amino]phenyl}-N-(2- |
| | methylbenzyl)-1H-pyrazole-1-carboxamide |
| 110 | 5-cyclopropyl-3-{2-hydroxy-4-[(2-methoxybenzoyl)amino]phenyl}-N-(3- |
| | methylbenzyl)-1H-pyrazole-1-carboxamide |
| 111 | 5-cyclopropyl-3-{2-hydroxy-4-[(2-methoxybenzoyl)amino]phenyl}-N-(4- |
| | methylbenzyl)-1H-pyrazole-1-carboxamide |
| 112 | N-(2-chlorobenzyl)-5-cyclopropyl-3-{2-hydroxy-4-[(2- |
| | methoxybenzoyl)amino]phenyl}-1H-pyrazole-1-carboxamide |
| 113 | 5-cyclopropyl-N-(4-fluorobenzyl)-3-{2-hydroxy-4-[(2- |
| | methoxybenzoyl)amino]phenyl}-1H-pyrazole-1-carboxamide |
| 114 | 5-cyclopropyl-N-(3,4-dichlorobenzyl)-3-{2-hydroxy-4-[(2- |
| | methoxybenzoyl)amino]phenyl}-1H-pyrazole-1-carboxamide |
| 115 | 5-cyclopropyl-3-{2-hydroxy-4-[(2-methoxybenzoyl)amino]phenyl}-N-(4- |
| | methoxybenzyl)-1H-pyrazole-1-carboxamide |
| 116 | N-(sec-butyl)-5-cyclopropyl-3-{2-hydroxy-4-[(2-methoxybenzoyl)amino]phenyl}- |
| | 1H-pyrazole-1-carboxamide |
| 117 | 5-cyclopropyl-N-(2-fluorobenzyl)-3-{2-hydroxy-4-[(2- |
| | methoxybenzoyl)amino]phenyl}-1H-pyrazole-1-carboxamide |
| 118 | 5-cyclopropyl-N-(3-fluorobenzyl)-3-{2-hydroxy-4-[(2- |
| | methoxybenzoyl)amino]phenyl}-1H-pyrazole-1-carboxamide |
| 119 | N,5-dicyclopropyl-3-{2-hydroxy-4-[(2-methoxybenzoyl)amino]phenyl}-1H- |
| | pyrazole-1-carboxamide |
| 120 | 5-cyclobutyl-3-{2-hydroxy-4-[(2-methoxybenzoyl)amino]phenyl}-N-isopropyl- |
| | 1H-pyrazole-1-carboxamide |
| 121 | 5-cyclobutyl-N-ethyl-3-{2-hydroxy-4-[(2-methoxybenzoyl)amino]phenyl}-1H- |
| | pyrazole-1-carboxamide |
| 122 | N-allyl-5-cyclobutyl-3-{2-hydroxy-4-[(2-methoxybenzoyl)amino]phenyl}-1H- |
| | pyrazole-1-carboxamide |
| 123 | 5-cyclobutyl-3-{2-hydroxy-4-[(2-methoxybenzoyl)amino]phenyl}-N-propyl-1H- |
| | pyrazole-1-carboxamide |
| 124 | N-benzyl-5-cyclobutyl-3-{2-hydroxy-4-[(2-methoxybenzoyl)amino]phenyl}-1H- |
| | pyrazole-1-carboxamide |
| 125 | 5-cyclobutyl-3-{2-hydroxy-4-[(2-methoxybenzoyl)amino]phenyl}-N-(2- |
| | phenylethyl)-1H-pyrazole-1-carboxamide |
| 126 | 5-cyclobutyl-3-{2-hydroxy-4-[(2-methoxybenzoyl)amino]phenyl}-N-(2- |
| | methylbenzyl)-1H-pyrazole-1-carboxamide |
| 127 | 5-cyclobutyl-3-{2-hydroxy-4-[(2-methoxybenzoyl)amino]phenyl}-N-(3- |
| | methylbenzyl)-1H-pyrazole-1-carboxamide |
| 128 | 5-cyclobutyl-3-{2-hydroxy-4-[(2-methoxybenzoyl)amino]phenyl}-N-(4- |
| | methylbenzyl)-1H-pyrazole-1-carboxamide |
| 129 | N-(2-chlorobenzyl)-5-cyclobutyl-3-{2-hydroxy-4-[(2- |
| | methoxybenzoyl)amino]phenyl}-1H-pyrazole-1-carboxamide |
| 130 | 5-cyclobutyl-N-(4-fluorobenzyl)-3-{2-hydroxy-4-[(2- |
| | methoxybenzoyl)amino]phenyl}-1H-pyrazole-1-carboxamide |
| 131 | 5-cyclobutyl-N-(3,4-dichlorobenzyl)-3-{2-hydroxy-4-[(2- |
| | methoxybenzoyl)amino]phenyl}-1H-pyrazole-1-carboxamide |
| 132 | 5-cyclobutyl-3-{2-hydroxy-4-[(2-methoxybenzoyl)amino]phenyl}-N-(4- |
| | methoxybenzyl)-1H-pyrazole-1-carboxamide |
| 133 | N-(sec-butyl)-5-cyclobutyl-3-{2-hydroxy-4-[(2-methoxybenzoyl)amino]phenyl}- |
| | 1H-pyrazole-1-carboxamide |
| 134 | 5-cyclobutyl-N-(2-fluorobenzyl)-3-{2-hydroxy-4-[(2- |
| | methoxybenzoyl)amino]phenyl}-1H-pyrazole-1-carboxamide |
| 135 | 5-cyclobutyl-N-(3-fluorobenzyl)-3-{2-hydroxy-4-[(2- |
| | methoxybenzoyl)amino]phenyl}-1H-pyrazole-1-carboxamide |
| 136 | 5-cyclobutyl-N-cyclopropyl-3-{2-hydroxy-4-[(2-methoxybenzoyl)amino]phenyl}- |
| | 1H-pyrazole-1-carboxamide |
| 137 | 5-cyclopentyl-3-{2-hydroxy-4-[(2-methoxybenzoyl)amino]phenyl}-N-isopropyl- |
| | 1H-pyrazole-1-carboxamide |
| 138 | 5-cyclopentyl-N-ethyl-3-{2-hydroxy-4-[(2-methoxybenzoyl)amino]phenyl}-1H- |
| | pyrazole-1-carboxamide |
| 139 | N-allyl-5-cyclopentyl-3-{2-hydroxy-4-[(2-methoxybenzoyl)amino]phenyl}-1H- |
| | pyrazole-1-carboxamide |
| 140 | 5-cyclopentyl-3-{2-hydroxy-4-[(2-methoxybenzoyl)amino]phenyl}-N-propyl-1H- |
| | pyrazole-1-carboxamide |
| 141 | N-benzyl-5-cyclopentyl-3-{2-hydroxy-4-[(2-methoxybenzoyl)amino]phenyl}-1H- |
| | pyrazole-1-carboxamide |
| 142 | 5-cyclopentyl-3-{2-hydroxy-4-[(2-methoxybenzoyl)amino]phenyl}-N-(2- |
| | phenylethyl)-1H-pyrazole-1-carboxamide |
| 143 | 5-cyclopentyl-3-{2-hydroxy-4-[(2-methoxybenzoyl)amino]phenyl}-N-(2- |
| | methylbenzyl)-1H-pyrazole-1-carboxamide |
| 144 | 5-cyclopentyl-3-{2-hydroxy-4-[(2-methoxybenzoyl)amino]phenyl}-N-(3- |
| | methylbenzyl)-1H-pyrazole-1-carboxamide |
| 145 | 5-cyclopentyl-3-{2-hydroxy-4-[(2-methoxybenzoyl)amino]phenyl}-N-(4- |
| | methylbenzyl)-1H-pyrazole-1-carboxamide |
| 146 | N-(2-chlorobenzyl)-5-cyclopentyl-3-{2-hydroxy-4-[(2- |
| | methoxybenzoyl)amino]phenyl}-1H-pyrazole-1-carboxamide |
| 147 | 5-cyclopentyl-N-(4-fluorobenzyl)-3-{2-hydroxy-4-[(2- |
| | methoxybenzoyl)amino]phenyl}-1H-pyrazole-1-carboxamide |
| 148 | 5-cyclopentyl-N-(3,4-dichlorobenzyl)-3-{2-hydroxy-4-[(2- |
| | methoxybenzoyl)amino]phenyl}-1H-pyrazole-1-carboxamide |
| 149 | 5-cyclopentyl-3-{2-hydroxy-4-[(2-methoxybenzoyl)amino]phenyl}-N-(4- |
| | methoxybenzyl)-1H-pyrazole-1-carboxamide |
| 150 | N-(sec-butyl)-5-cyclopentyl-3-{2-hydroxy-4-[(2-methoxybenzoyl)amino]phenyl}- |
| | 1H-pyrazole-1-carboxamide |
| 151 | 5-cyclopentyl-N-(2-fluorobenzyl)-3-{2-hydroxy-4-[(2- |
| | methoxybenzoyl)amino]phenyl}-1H-pyrazole-1-carboxamide |
| 152 | 5-cyclopentyl-N-(3-fluorobenzyl)-3-{2-hydroxy-4-[(2- |
| | methoxybenzoyl)amino]phenyl}-1H-pyrazole-1-carboxamide |
| 153 | 5-cyclopentyl-N-cyclopropyl-3-{2-hydroxy-4-[(2- |
| | methoxybenzoyl)amino]phenyl}-1H-pyrazole-1-carboxamide |
| 154 | 5-cyclopropyl-3-{2-hydroxy-4-[(2-methylbenzoyl)amino]phenyl}-N-isopropyl- |
| | 1H-pyrazole-1-carboxamide |
| 155 | 5-cyclopropyl-N-ethyl-3-{2-hydroxy-4-[(2-methylbenzoyl)amino]phenyl}-1H- |
| | pyrazole-1-carboxamide |
| 156 | N-allyl-5-cyclopropyl-3-{2-hydroxy-4-[(2-methylbenzoyl)amino]phenyl}-1H- |
| | pyrazole-1-carboxamide |
| 157 | 5-cyclopropyl-3-{2-hydroxy-4-[(2-methylbenzoyl)amino]phenyl}-N-propyl-1H- |
| | pyrazole-1-carboxamide |
| 158 | N-benzyl-5-cyclopropyl-3-{2-hydroxy-4-[(2-methylbenzoyl)amino]phenyl}-1H- |
| | pyrazole-1-carboxamide |
| 159 | 5-cyclopropyl-3-{2-hydroxy-4-[(2-methylbenzoyl)amino]phenyl}-N-(2- |
| | phenylethyl)-1H-pyrazole-1-carboxamide |
| 160 | 5-cyclopropyl-3-{2-hydroxy-4-[(2-methylbenzoyl)amino]phenyl}-N-(2- |
| | methylbenzyl)-1H-pyrazole-1-carboxamide |
| 161 | 5-cyclopropyl-3-{2-hydroxy-4-[(2-methylbenzoyl)amino]phenyl}-N-(3- |
| | methylbenzyl)-1H-pyrazole-1-carboxamide |
| 162 | 5-cyclopropyl-3-{2-hydroxy-4-[(2-methylbenzoyl)amino]phenyl}-N-(4- |
| | methylbenzyl)-1H-pyrazole-1-carboxamide |
| 163 | N-(2-chlorobenzyl)-5-cyclopropyl-3-{2-hydroxy-4-[(2- |
| | methylbenzoyl)amino]phenyl}-1H-pyrazole-1-carboxamide |
| 164 | 5-cyclopropyl-N-(4-fluorobenzyl)-3-{2-hydroxy-4-[(2- |
| | methylbenzoyl)amino]phenyl}-1H-pyrazole-1-carboxamide |
| 165 | 5-cyclopropyl-N-(3,4-dichlorobenzyl)-3-{2-hydroxy-4-[(2- |
| | methylbenzoyl)amino]phenyl}-1H-pyrazole-1-carboxamide |
| 166 | 5-cyclopropyl-3-{2-hydroxy-4-[(2-methylbenzoyl)amino]phenyl}-N-(4- |
| | methoxybenzyl)-1H-pyrazole-1-carboxamide |
| 167 | N-(sec-butyl)-5-cyclopropyl-3-{2-hydroxy-4-[(2-methylbenzoyl)amino]phenyl}- |
| | 1H-pyrazole-1-carboxamide |
| 168 | 5-cyclopropyl-N-(2-fluorobenzyl)-3-{2-hydroxy-4-[(2- |
| | methylbenzoyl)amino]phenyl}-1H-pyrazole-1-carboxamide |
| 169 | 5-cyclopropyl-N-(3-fluorobenzyl)-3-{2-hydroxy-4-[(2- |
| | methylbenzoyl)amino]phenyl}-1H-pyrazole-1-carboxamide |
| 170 | N,5-dicyclopropyl-3-{2-hydroxy-4-[(2-methylbenzoyl)amino]phenyl}-1H- |
| | pyrazole-1-carboxamide |
| 171 | 5-cyclobutyl-3-{2-hydroxy-4-[(2-methylbenzoyl)amino]phenyl}-N-isopropyl-1H- |
| | pyrazole-1-carboxamide |
| 172 | 5-cyclobutyl-N-ethyl-3-{2-hydroxy-4-[(2-methylbenzoyl)amino]phenyl}-1H- |
| | pyrazole-1-carboxamide |
| 173 | N-allyl-5-cyclobutyl-3-{2-hydroxy-4-[(2-methylbenzoyl)amino]phenyl}-1H- |
| | pyrazole-1-carboxamide |
| 174 | 5-cyclobutyl-3-{2-hydroxy-4-[(2-methylbenzoyl)amino]phenyl}-N-propyl-1H- |
| | pyrazole-1-carboxamide |
| 175 | N-benzyl-5-cyclobutyl-3-{2-hydroxy-4-[(2-methylbenzoyl)amino]phenyl}-1H- |
| | pyrazole-1-carboxamide |
| 176 | 5-cyclobutyl-3-{2-hydroxy-4-[(2-methylbenzoyl)amino]phenyl}-N-(2- |
| | phenylethyl)-1H-pyrazole-1-carboxamide |
| 177 | 5-cyclobutyl-3-{2-hydroxy-4-[(2-methylbenzoyl)amino]phenyl}-N-(2- |
| | methylbenzyl)-1H-pyrazole-1-carboxamide |
| 178 | 5-cyclobutyl-3-{2-hydroxy-4-[(2-methylbenzoyl)amino]phenyl}-N-(3- |
| | methylbenzyl)-1H-pyrazole-1-carboxamide |
| 179 | 5-cyclobutyl-3-{2-hydroxy-4-[(2-methylbenzoyl)amino]phenyl}-N-(4- |
| | methylbenzyl)-1H-pyrazole-1-carboxamide |
| 180 | N-(2-chlorobenzyl)-5-cyclobutyl-3-{2-hydroxy-4-[(2- |
| | methylbenzoyl)amino]phenyl}-1H-pyrazole-1-carboxamide |
| 181 | 5-cyclobutyl-N-(4-fluorobenzyl)-3-{2-hydroxy-4-[(2- |
| | methylbenzoyl)amino]phenyl}-1H-pyrazole-1-carboxamide |
| 182 | 5-cyclobutyl-N-(3,4-dichlorobenzyl)-3-{2-hydroxy-4-[(2- |
| | methylbenzoyl)amino]phenyl}-1H-pyrazole-1-carboxamide |
| 183 | 5-cyclobutyl-3-{2-hydroxy-4-[(2-methylbenzoyl)amino]phenyl}-N-(4- |
| | methoxybenzyl)-1H-pyrazole-1-carboxamide |
| 184 | N-(sec-butyl)-5-cyclobutyl-3-{2-hydroxy-4-[(2-methylbenzoyl)amino]phenyl}- |
| | 1H-pyrazole-1-carboxamide |
| 185 | 5-cyclobutyl-N-(2-fluorobenzyl)-3-{2-hydroxy-4-[(2- |
| | methylbenzoyl)amino]phenyl}-1H-pyrazole-1-carboxamide |
| 186 | 5-cyclobutyl-N-(3-fluorobenzyl)-3-{2-hydroxy-4-[(2- |
| | methylbenzoyl)amino]phenyl}-1H-pyrazole-1-carboxamide |
| 187 | 5-cyclobutyl-N-cyclopropyl-3-{2-hydroxy-4-[(2-methylbenzoyl)amino]phenyl}- |
| | 1H-pyrazole-1-carboxamide |
| 188 | 5-cyclopentyl-3-{2-hydroxy-4-[(2-methylbenzoyl)amino]phenyl}-N-isopropyl- |
| | 1H-pyrazole-1-carboxamide |
| 189 | 5-cyclopentyl-N-ethyl-3-{2-hydroxy-4-[(2-methylbenzoyl)amino]phenyl}-1H- |
| | pyrazole-1-carboxamide |
| 190 | N-allyl-5-cyclopentyl-3-{2-hydroxy-4-[(2-methylbenzoyl)amino]phenyl}-1H- |
| | pyrazole-1-carboxamide |
| 191 | 5-cyclopentyl-3-{2-hydroxy-4-[(2-methylbenzoyl)amino]phenyl}-N-propyl-1H- |
| | pyrazole-1-carboxamide |
| 192 | N-benzyl-5-cyclopentyl-3-{2-hydroxy-4-[(2-methylbenzoyl)amino]phenyl}-1H- |
| | pyrazole-1-carboxamide |
| 193 | 5-cyclopentyl-3-{2-hydroxy-4-[(2-methylbenzoyl)amino]phenyl}-N-(2- |
| | phenylethyl)-1H-pyrazole-1-carboxamide |
| 194 | 5-cyclopentyl-3-{2-hydroxy-4-[(2-methylbenzoyl)amino]phenyl}-N-(2- |
| | methylbenzyl)-1H-pyrazole-1-carboxamide |
| 195 | 5-cyclopentyl-3-{2-hydroxy-4-[(2-methylbenzoyl)amino]phenyl}-N-(3- |
| | methylbenzyl)-1H-pyrazole-1-carboxamide |
| 196 | 5-cyclopentyl-3-{2-hydroxy-4-[(2-methylbenzoyl)amino]phenyl}-N-(4- |
| | methylbenzyl)-1H-pyrazole-1-carboxamide |
| 197 | N-(2-chlorobenzyl)-5-cyclopentyl-3-{2-hydroxy-4-[(2- |
| | methylbenzoyl)amino]phenyl}-1H-pyrazole-1-carboxamide |
| 198 | 5-cyclopentyl-N-(4-fluorobenzyl)-3-{2-hydroxy-4-[(2- |
| | methylbenzoyl)amino]phenyl}-1H-pyrazole-1-carboxamide |
| 199 | 5-cyclopentyl-N-(3,4-dichlorobenzyl)-3-{2-hydroxy-4-[(2- |
| | methylbenzoyl)amino]phenyl}-1H-pyrazole-1-carboxamide |
| 200 | 5-cyclopentyl-3-{2-hydroxy-4-[(2-methylbenzoyl)amino]phenyl}-N-(4- |
| | methoxybenzyl)-1H-pyrazole-1-carboxamide |
| 201 | N-(sec-butyl)-5-cyclopentyl-3-{2-hydroxy-4-[(2-methylbenzoyl)amino]phenyl}- |
| | 1H-pyrazole-1-carboxamide |
| 202 | 5-cyclopentyl-N-(2-fluorobenzyl)-3-{2-hydroxy-4-[(2- |
| | methylbenzoyl)amino]phenyl}-1H-pyrazole-1-carboxamide |
| 203 | 5-cyclopentyl-N-(3-fluorobenzyl)-3-{2-hydroxy-4-[(2- |
| | methylbenzoyl)amino]phenyl}-1H-pyrazole-1-carboxamide |
| 204 | 5-cyclopentyl-N-cyclopropyl-3-{2-hydroxy-4-[(2-methylbenzoyl)amino]phenyl}- |
| | 1H-pyrazole-1-carboxamide |
| 205 | 5-cyclopropyl-3-(2-hydroxy-4-{[3-(trifluoromethyl)benzoyl]amino}phenyl)-N- |
| | isopropyl-1H-pyrazole-1-carboxamide |
| 206 | 5-cyclopropyl-N-ethyl-3-(2-hydroxy-4-{[3- |
| | (trifluoromethyl)benzoyl]amino}phenyl)-1H-pyrazole-1-carboxamide |
| 207 | N-allyl-5-cyclopropyl-3-(2-hydroxy-4-{[3- |
| | (trifluoromethyl)benzoyl]amino}phenyl)-1H-pyrazole-1-carboxamide |
| 208 | 5-cyclopropyl-3-(2-hydroxy-4-{[3-(trifluoromethyl)benzoyl]amino}phenyl)-N- |
| | propyl-1H-pyrazole-1-carboxamide |
| 209 | N-benzyl-5-cyclopropyl-3-(2-hydroxy-4-{[3- |
| | (trifluoromethyl)benzoyl]amino}phenyl)-1H-pyrazole-1-carboxamide |
| 210 | 5-cyclopropyl-3-(2-hydroxy-4-{[3-(trifluoromethyl)benzoyl]amino}phenyl)-N-(2- |
| | phenylethyl)-1H-pyrazole-1-carboxamide |
| 211 | 5-cyclopropyl-3-(2-hydroxy-4-{[3-(trifluoromethyl)benzoyl]amino}phenyl)-N-(2- |
| | methylbenzyl)-1H-pyrazole-1-carboxamide |
| 212 | 5-cyclopropyl-3-(2-hydroxy-4-{[3-(trifluoromethyl)benzoyl]amino}phenyl)-N-(3- |
| | methylbenzyl)-1H-pyrazole-1-carboxamide |
| 213 | 5-cyclopropyl-3-(2-hydroxy-4-{[3-(trifluoromethyl)benzoyl]amino}phenyl)-N-(4- |
| | methylbenzyl)-1H-pyrazole-1-carboxamide |
| 214 | N-(2-chlorobenzyl)-5-cyclopropyl-3-(2-hydroxy-4-{[3- |
| | (trifluoromethyl)benzoyl]amino}phenyl)-1H-pyrazole-1-carboxamide |
| 215 | 5-cyclopropyl-N-(4-fluorobenzyl)-3-(2-hydroxy-4-{[3- |
| | (trifluoromethyl)benzoyl]amino}phenyl)-1H-pyrazole-1-carboxamide |
| 216 | 5-cyclopropyl-N-(3,4-dichlorobenzyl)-3-(2-hydroxy-4-{[3- |
| | (trifluoromethyl)benzoyl]amino}phenyl)-1H-pyrazole-1-carboxamide |
| 217 | 5-cyclopropyl-3-(2-hydroxy-4-{[3-(trifluoromethyl)benzoyl]amino}phenyl)-N-(4- |
| | methoxybenzyl)-1H-pyrazole-1-carboxamide |
| 218 | N-(sec-butyl)-5-cyclopropyl-3-(2-hydroxy-4-{[3- |
| | (trifluoromethyl)benzoyl]amino}phenyl)-1H-pyrazole-1-carboxamide |
| 219 | 5-cyclopropyl-N-(2-fluorobenzyl)-3-(2-hydroxy-4-{[3- |
| | (trifluoromethyl)benzoyl]amino}phenyl)-1H-pyrazole-1-carboxamide |
| 220 | N-allyl-5-cyclobutyl-3-(2-hydroxy-4-{[3- |
| | (trifluoromethyl)benzoyl]amino}phenyl)-1H-pyrazole-1-carboxamide |
| 221 | N,5-dicyclopropyl-3-(2-hydroxy-4-{[3-(trifluoromethyl)benzoyl]amino}phenyl)- |
| | 1H-pyrazole-1-carboxamide |
| 222 | 5-cyclobutyl-3-(2-hydroxy-4-{[3-(trifluoromethyl)benzoyl]amino}phenyl)-N- |
| | isopropyl-1H-pyrazole-1-carboxamide |
| 223 | 5-cyclobutyl-N-ethyl-3-(2-hydroxy-4-{[3- |
| | (trifluoromethyl)benzoyl]amino}phenyl)-1H-pyrazole-1-carboxamide |
| 224 | N-allyl-5-cyclobutyl-3-(2-hydroxy-4-{[3- |
| | (trifluoromethyl)benzoyl]amino}phenyl)-1H-pyrazole-1-carboxamide |
| 225 | 5-cyclobutyl-3-(2-hydroxy-4-{[3-(trifluoromethyl)benzoyl]amino}phenyl)-N- |
| | propyl-1H-pyrazole-1-carboxamide |
| 226 | N-benzyl-5-cyclobutyl-3-(2-hydroxy-4-{[3- |
| | (trifluoromethyl)benzoyl]amino}phenyl)-1H-pyrazole-1-carboxamide |
| 227 | 5-cyclobutyl-3-(2-hydroxy-4-{[3-(trifluoromethyl)benzoyl]amino}phenyl)-N-(2- |
| | phenylethyl)-1H-pyrazole-1-carboxamide |
| 228 | 5-cyclobutyl-3-(2-hydroxy-4-{[3-(trifluoromethyl)benzoyl]amino}phenyl)-N-(2- |
| | methylbenzyl)-1H-pyrazole-1-carboxamide |
| 229 | 5-cyclobutyl-3-(2-hydroxy-4-{[3-(trifluoromethyl)benzoyl]amino}phenyl)-N-(3- |
| | methylbenzyl)-1H-pyrazole-1-carboxamide |
| 230 | 5-cyclobutyl-3-(2-hydroxy-4-{[3-(trifluoromethyl)benzoyl]amino}phenyl)-N-(4- |
| | methylbenzyl)-1H-pyrazole-1-carboxamide |
| 231 | N-(2-chlorobenzyl)-5-cyclobutyl-3-(2-hydroxy-4-{[3- |
| | (trifluoromethyl)benzoyl]amino}phenyl)-1H-pyrazole-1-carboxamide |
| 232 | 5-cyclobutyl-N-(4-fluorobenzyl)-3-(2-hydroxy-4-{[3- |
| | (trifluoromethyl)benzoyl]amino}phenyl)-1H-pyrazole-1-carboxamide |
| 233 | 5-cyclobutyl-N-(3,4-dichlorobenzyl)-3-(2-hydroxy-4-{[3- |
| | (trifluoromethyl)benzoyl]amino}phenyl)-1H-pyrazole-1-carboxamide |
| 234 | 5-cyclobutyl-3-(2-hydroxy-4-{[3-(trifluoromethyl)benzoyl]amino}phenyl)-N-(4- |
| | methoxybenzyl)-1H-pyrazole-1-carboxamide |
| 235 | N-(sec-butyl)-5-cyclobutyl-3-(2-hydroxy-4-{[3- |
| | (trifluoromethyl)benzoyl]amino}phenyl)-1H-pyrazole-1-carboxamide |
| 236 | 5-cyclobutyl-N-(2-fluorobenzyl)-3-(2-hydroxy-4-{[3- |
| | (trifluoromethyl)benzoyl]amino}phenyl)-1H-pyrazole-1-carboxamide |
| 237 | 5-cyclobutyl-N-(3-fluorobenzyl)-3-(2-hydroxy-4-{[3- |
| | (trifluoromethyl)benzoyl]amino}phenyl)-1H-pyrazole-1-carboxamide |
| 238 | 5-cyclobutyl-N-cyclopropyl-3-(2-hydroxy-4-{[3- |
| | (trifluoromethyl)benzoyl]amino}phenyl)-1H-pyrazole-1-carboxamide |
| 239 | 5-cyclopentyl-3-(2-hydroxy-4-{[3-(trifluoromethyl)benzoyl]amino}phenyl)-N- |
| | isopropyl-1H-pyrazole-1-carboxamide |
| 240 | 5-cyclopentyl-N-ethyl-3-(2-hydroxy-4-{[3- |
| | (trifluoromethyl)benzoyl]amino}phenyl)-1H-pyrazole-1-carboxamide |
| 241 | N-allyl-5-cyclopentyl-3-(2-hydroxy-4-{[3- |
| | (trifluoromethyl)benzoyl]amino}phenyl)-1H-pyrazole-1-carboxamide |
| 242 | 5-cyclopentyl-3-(2-hydroxy-4-{[3-(trifluoromethyl)benzoyl]amino}phenyl)-N- |
| | propyl-1H-pyrazole-1-carboxamide |
| 243 | N-benzyl-5-cyclopentyl-3-(2-hydroxy-4-{[3- |
| | (trifluoromethyl)benzoyl]amino}phenyl)-1H-pyrazole-1-carboxamide |
| 244 | 5-cyclopentyl-3-(2-hydroxy-4-{[3-(trifluoromethyl)benzoyl]amino}phenyl)-N-(2- |
| | phenylethyl)-1H-pyrazole-1-carboxamide |
| 245 | 5-cyclopentyl-3-(2-hydroxy-4-{[3-(trifluoromethyl)benzoyl]amino}phenyl)-N-(2- |
| | methylbenzyl)-1H-pyrazole-1-carboxamide |
| 246 | 5-cyclopentyl-3-(2-hydroxy-4-{[3-(trifluoromethyl)benzoyl]amino}phenyl)-N-(3- |
| | methylbenzyl)-1H-pyrazole-1-carboxamide |
| 247 | 5-cyclopentyl-3-(2-hydroxy-4-{[3-(trifluoromethyl)benzoyl]amino}phenyl)-N-(4- |
| | methylbenzyl)-1H-pyrazole-1-carboxamide |
| 248 | N-(2-chlorobenzyl)-5-cyclopentyl-3-(2-hydroxy-4-{[3- |
| | (trifluoromethyl)benzoyl]amino}phenyl)-1H-pyrazole-1-carboxamide |
| 249 | 5-cyclopentyl-N-(4-fluorobenzyl)-3-(2-hydroxy-4-{[3- |
| | (trifluoromethyl)benzoyl]amino}phenyl)-1H-pyrazole-1-carboxamide |
| 250 | 5-cyclopentyl-N-(3,4-dichlorobenzyl)-3-(2-hydroxy-4-{[3- |
| | (trifluoromethyl)benzoyl]amino}phenyl)-1H-pyrazole-1-carboxamide |
| 251 | 5-cyclopentyl-3-(2-hydroxy-4-{[3-(trifluoromethyl)benzoyl]amino}phenyl)-N-(4- |
| | methoxybenzyl)-1H-pyrazole-1-carboxamide |
| 252 | N-(sec-butyl)-5-cyclopentyl-3-(2-hydroxy-4-{[3- |
| | (trifluoromethyl)benzoyl]amino}phenyl)-1H-pyrazole-1-carboxamide |
| 253 | 5-cyclopentyl-N-(2-fluorobenzyl)-3-(2-hydroxy-4-{[3- |
| | (trifluoromethyl)benzoyl]amino}phenyl)-1H-pyrazole-1-carboxamide |
| 254 | 5-cyclopentyl-N-(3-fluorobenzyl)-3-(2-hydroxy-4-{[3- |
| | (trifluoromethyl)benzoyl]amino}phenyl)-1H-pyrazole-1-carboxamide |
| 255 | 5-cyclopentyl-N-cyclopropyl-3-(2-hydroxy-4-{[3- |
| | (trifluoromethyl)benzoyl]amino}phenyl)-1H-pyrazole-1-carboxamide |
| 256 | 3-{4-[(4-chlorobenzoyl)amino]-2-hydroxyphenyl}-5-cyclopropyl-N-isopropyl-1H- |
| | pyrazole-1-carboxamide |
| 257 | 3-{4-[(4-chlorobenzoyl)amino]-2-hydroxyphenyl}-5-cyclopropyl-N-ethyl-1H- |
| | pyrazole-1-carboxamide |
| 258 | N-allyl-3-{4-[(4-chlorobenzoyl)amino]-2-hydroxyphenyl}-5-cyclopropyl-1H- |
| | pyrazole-1-carboxamide |
| 259 | 3-{4-[(4-chlorobenzoyl)amino]-2-hydroxyphenyl}-5-cyclopropyl-N-propyl-1H- |
| | pyrazole-1-carboxamide |
| 260 | N-benzyl-3-{4-[(4-chlorobenzoyl)amino]-2-hydroxyphenyl}-5-cyclopropyl-1H- |
| | pyrazole-1-carboxamide |
| 261 | 3-{4-[(4-chlorobenzoyl)amino]-2-hydroxyphenyl}-5-cyclopropyl-N-(2- |
| | phenylethyl)-1H-pyrazole-1-carboxamide |
| 262 | 3-{4-[(4-chlorobenzoyl)amino]-2-hydroxyphenyl}-5-cyclopropyl-N-(4- |
| | fluorobenzyl)-1H-pyrazole-1-carboxamide |
| 263 | 3-{4-[(4-chlorobenzoyl)amino]-2-hydroxyphenyl}-5-cyclopropyl-N-(3- |
| | methylbenzyl)-1H-pyrazole-1-carboxamide |
| 264 | 3-{4-[(4-chlorobenzoyl)amino]-2-hydroxyphenyl}-5-cyclopropyl-N-(4- |
| | methylbenzyl)-1H-pyrazole-1-carboxamide |
| 265 | 3-{4-[(4-chlorobenzoyl)amino]-2-hydroxyphenyl}-N-(2-chlorobenzyl)-5- |
| | cyclopropyl-1H-pyrazole-1-carboxamide |
| 266 | 3-{4-[(4-chlorobenzoyl)amino]-2-hydroxyphenyl}-5-cyclopropyl-N-(4- |
| | fluorobenzyl)-1H-pyrazole-1-carboxamide |
| 267 | 3-{4-[(4-chlorobenzoyl)amino]-2-hydroxyphenyl}-5-cyclopropyl-N-(3,4- |
| | dichlorobenzyl)-1H-pyrazole-1-carboxamide |
| 268 | 3-{4-[(4-chlorobenzoyl)amino]-2-hydroxyphenyl}-5-cyclopropyl-N-(4- |
| | methoxybenzyl)-1H-pyrazole-1-carboxamide |
| 269 | N-(sec-butyl)-3-{4-[(4-chlorobenzoyl)amino]-2-hydroxyphenyl}-5-cyclopropyl- |
| | 1H-pyrazole-1-carboxamide |
| 270 | 3-{4-[(4-chlorobenzoyl)amino]-2-hydroxyphenyl}-5-cyclopropyl-N-(2- |
| | fluorobenzyl)-1H-pyrazole-1-carboxamide |
| 271 | 3-{4-[(4-chlorobenzoyl)amino]-2-hydroxyphenyl}-5-cyclopropyl-N-(3- |
| | fluorobenzyl)-1H-pyrazole-1-carboxamide |
| 272 | 3-{4-[(4-chlorobenzoyl)amino]-2-hydroxyphenyl}-N,5-dicyclopropyl-1H- |
| | pyrazole-1-carboxamide |
| 273 | 3-{4-[(4-chlorobenzoyl)amino]-2-hydroxyphenyl}-5-cyclobutyl-N-isopropyl-1H- |
| | pyrazole-1-carboxamide |
| 274 | 3-{4-[(4-chlorobenzoyl)amino]-2-hydroxyphenyl}-5-cyclobutyl-N-ethyl-1H- |
| | pyrazole-1-carboxamide |
| 275 | N-allyl-3-{4-[(4-chlorobenzoyl)amino]-2-hydroxyphenyl}-5-cyclobutyl-1H- |
| | pyrazole-1-carboxamide |
| 276 | 3-{4-[(4-chlorobenzoyl)amino]-2-hydroxyphenyl}-5-cyclobutyl-N-propyl-1H- |
| | pyrazole-1-carboxamide |
| 277 | N-benzyl-3-{4-[(4-chlorobenzoyl)amino]-2-hydroxyphenyl}-5-cyclobutyl-1H- |
| | pyrazole-1-carboxamide |
| 278 | 3-{4-[(4-chlorobenzoyl)amino]-2-hydroxyphenyl}-5-cyclobutyl-N-(2- |
| | phenylethyl)-1H-pyrazole-1-carboxamide |
| 279 | 3-{4-[(4-chlorobenzoyl)amino]-2-hydroxyphenyl}-5-cyclobutyl-N-(2- |
| | methylbenzyl)-1H-pyrazole-1-carboxamide |
| 280 | 3-{4-[(4-chlorobenzoyl)amino]-2-hydroxyphenyl}-5-cyclobutyl-N-(3- |
| | methylbenzyl)-1H-pyrazole-1-carboxamide |
| 281 | 3-{4-[(4-chlorobenzoyl)amino]-2-hydroxyphenyl}-5-cyclobutyl-N-(4- |
| | methylbenzyl)-1H-pyrazole-1-carboxamide |
| 282 | 3-{4-[(4-chlorobenzoyl)amino]-2-hydroxyphenyl}-N-(2-chlorobenzyl)-5- |
| | cyclobutyl-1H-pyrazole-1-carboxamide |
| 283 | 3-{4-[(4-chlorobenzoyl)amino]-2-hydroxyphenyl}-5-cyclobutyl-N-(4- |
| | fluorobenzyl)-1H-pyrazole-1-carboxamide |
| 284 | 3-{4-[(4-chlorobenzoyl)amino]-2-hydroxyphenyl}-5-cyclobutyl-N-(3,4- |
| | dichlorobenzyl)-1H-pyrazole-1-carboxamide |
| 285 | 3-{4-[(4-chlorobenzoyl)amino]-2-hydroxyphenyl}-5-cyclobutyl-N-(4- |
| | methoxybenzyl)-1H-pyrazole-1-carboxamide |
| 286 | N-(sec-butyl)-3-{4-[(4-chlorobenzoyl)amino]-2-hydroxyphenyl}-5-cyclobutyl-1H- |
| | pyrazole-1-carboxamide |
| 287 | 3-{4-[(4-chlorobenzoyl)amino]-2-hydroxyphenyl}-5-cyclobutyl-N-(2- |
| | fluorobenzyl)-1H-pyrazole-1-carboxamide |
| 288 | 3-{4-[(4-chlorobenzoyl)amino]-2-hydroxyphenyl}-5-cyclobutyl-N-(3- |
| | fluorobenzyl)-1H-pyrazole-1-carboxamide |
| 289 | 3-{4-[(4-chlorobenzoyl)amino]-2-hydroxyphenyl}-5-cyclobutyl-N-cyclopropyl- |
| | 1H-pyrazole-1-carboxamide |
| 290 | 3-{4-[(4-chlorobenzoyl)amino]-2-hydroxyphenyl}-5-cyclopentyl-N-isopropyl-1H- |
| | pyrazole-1-carboxamide |
| 291 | 3-{4-[(4-chlorobenzoyl)amino]-2-hydroxyphenyl}-5-cyclopentyl-N-ethyl-1H- |
| | pyrazole-1-carboxamide |
| 292 | N-allyl-3-{4-[(4-chlorobenzoyl)amino]-2-hydroxyphenyl}-5-cyclopentyl-1H- |
| | pyrazole-1-carboxamide |
| 293 | 3-{4-[(4-chlorobenzoyl)amino]-2-hydroxyphenyl}-5-cyclopentyl-N-propyl-1H- |
| | pyrazole-1-carboxamide |
| 294 | N-benzyl-3-{4-[(4-chlorobenzoyl)amino]-2-hydroxyphenyl}-5-cyclopentyl-1H- |
| | pyrazole-1-carboxamide |
| 295 | 3-{4-[(4-chlorobenzoyl)amino]-2-hydroxyphenyl}-5-cyclopentyl-N-(2- |
| | phenylethyl)-1H-pyrazole-1-carboxamide |
| 296 | 3-{4-[(4-chlorobenzoyl)amino]-2-hydroxyphenyl}-5-cyclopentyl-N-(2- |
| | methylbenzyl)-1H-pyrazole-1-carboxamide |
| 297 | 3-{4-[(4-chlorobenzoyl)amino]-2-hydroxyphenyl}-5-cyclopentyl-N-(3- |
| | methylbenzyl)-1H-pyrazole-1-carboxamide |
| 298 | 3-{4-[(4-chlorobenzoyl)amino]-2-hydroxyphenyl}-5-cyclopentyl-N-(4- |
| | methylbenzyl)-1H-pyrazole-1-carboxamide |
| 299 | 3-{4-[(4-chlorobenzoyl)amino]-2-hydroxyphenyl}-N-(2-chlorobenzyl)-5- |
| | cyclopentyl-1H-pyrazole-1-carboxamide |
| 300 | 3-{4-[(4-chlorobenzoyl)amino]-2-hydroxyphenyl}-5-cyclopentyl-N-(4- |
| | fluorobenzyl)-1H-pyrazole-1-carboxamide |
| 301 | 3-{4-[(4-chlorobenzoyl)amino]-2-hydroxyphenyl}-5-cyclopentyl-N-(3,4- |
| | dichlorobenzyl)-1H-pyrazole-1-carboxamide |
| 302 | 3-{4-[(4-chlorobenzoyl)amino]-2-hydroxyphenyl}-5-cyclopentyl-N-(4- |
| | methoxybenzyl)-1H-pyrazole-1-carboxamide |
| 303 | N-(sec-butyl)-3-{4-[(4-chlorobenzoyl)amino]-2-hydroxyphenyl}-5-cyclopentyl- |
| | 1H-pyrazole-1-carboxamide |
| 304 | 3-{4-[(4-chlorobenzoyl)amino]-2-hydroxyphenyl}-5-cyclopentyl-N-(2- |
| | fluorobenzyl)-1H-pyrazole-1-carboxamide |
| 305 | 3-{4-[(4-chlorobenzoyl)amino]-2-hydroxyphenyl}-5-cyclopentyl-N-(3- |
| | fluorobenzyl)-1H-pyrazole-1-carboxamide |
| 306 | 3-{4-[(4-chlorobenzoyl)amino]-2-hydroxyphenyl}-5-cyclopentyl-N-cyclopropyl- |
| | 1H-pyrazole-1-carboxamide |
| 307 | 5-cyclopropyl-3-{4-[(4-ethoxybenzoyl)amino]-2-hydroxyphenyl}-N-isopropyl- |
| | 1H-pyrazole-1-carboxamide |
| 308 | 5-cyclopropyl-3-{4-[(4-ethoxybenzoyl)amino]-2-hydroxyphenyl}-N-ethyl-1H- |
| | pyrazole-1-carboxamide |
| 309 | N-allyl-5-cyclopropyl-3-{4-[(4-ethoxybenzoyl)amino]-2-hydroxyphenyl}-1H- |
| | pyrazole-1-carboxamide |
| 310 | 5-cyclopropyl-3-{4-[(4-ethoxybenzoyl)amino]-2-hydroxyphenyl}-N-propyl-1H- |
| | pyrazole-1-carboxamide |
| 311 | N-benzyl-5-cyclopropyl-3-{4-[(4-ethoxybenzoyl)amino]-2-hydroxyphenyl}-1H- |
| | pyrazole-1-carboxamide |
| 312 | 5-cyclopropyl-3-{4-[(4-ethoxybenzoyl)amino]-2-hydroxyphenyl}-N-(2- |
| | phenylethyl)-1H-pyrazole-1-carboxamide |
| 313 | 5-cyclopropyl-3-{4-[(4-ethoxybenzoyl)amino]-2-hydroxyphenyl}-N-(2- |
| | methylbenzyl)-1H-pyrazole-1-carboxamide |
| 314 | 5-cyclopropyl-3-{4-[(4-ethoxybenzoyl)amino]-2-hydroxyphenyl}-N-(3- |
| | methylbenzyl)-1H-pyrazole-1-carboxamide |
| 315 | 5-cyclopropyl-3-{4-[(4-ethoxybenzoyl)amino]-2-hydroxyphenyl}-N-(4- |
| | methylbenzyl)-1H-pyrazole-1-carboxamide |
| 316 | N-(2-chlorobenzyl)-5-cyclopropyl-3-{4-[(4-ethoxybenzoyl)amino]-2- |
| | hydroxyphenyl}-1H-pyrazole-1-carboxamide |
| 317 | 5-cyclopropyl-3-{4-[(4-ethoxybenzoyl)amino]-2-hydroxyphenyl}-N-(4- |
| | fluorobenzyl)-1H-pyrazole-1-carboxamide |
| 318 | 5-cyclopropyl-N-(3,4-dichlorobenzyl)-3-{4-[(4-ethoxybenzoyl)amino]-2- |
| | hydroxyphenyl}-1H-pyrazole-1-carboxamide |
| 319 | 5-cyclopropyl-3-{4-[(4-ethoxybenzoyl)amino]-2-hydroxyphenyl}-N-(4- |
| | methoxybenzyl)-1H-pyrazole-1-carboxamide |
| 320 | N-(sec-butyl)-5-cyclopropyl-3-{4-[(4-ethoxybenzoyl)amino]-2-hydroxyphenyl}- |
| | 1H-pyrazole-1-carboxamide |
| 321 | 5-cyclopropyl-3-{4-[(4-ethoxybenzoyl)amino]-2-hydroxyphenyl}-N-(2- |
| | fluorobenzyl)-1H-pyrazole-1-carboxamide |
| 322 | 5-cyclopropyl-3-{4-[(4-ethoxybenzoyl)amino]-2-hydroxyphenyl}-N-(3- |
| | fluorobenzyl)-1H-pyrazole-1-carboxamide |
| 323 | N,5-dicyclopropyl-3-{4-[(4-ethoxybenzoyl)amino]-2-hydroxyphenyl}-1H- |
| | pyrazole-1-carboxamide |
| 324 | 5-cyclobutyl-3-{4-[(4-ethoxybenzoyl)amino]-2-hydroxyphenyl}-N-isopropyl-1H- |
| | pyrazole-1-carboxamide |
| 325 | 5-cyclobutyl-3-{4-[(4-ethoxybenzoyl)amino]-2-hydroxyphenyl}-N-ethyl-1H- |
| | pyrazole-1-carboxamide |
| 326 | N-allyl-5-cyclobutyl-3-{4-[(4-ethoxybenzoyl)amino]-2-hydroxyphenyl}-1H- |
| | pyrazole-1-carboxamide |
| 327 | 5-cyclobutyl-3-{4-[(4-ethoxybenzoyl)amino]-2-hydroxyphenyl}-N-propyl-1H- |
| | pyrazole-1-carboxamide |
| 328 | N-benzyl-5-cyclobutyl-3-{4-[(4-ethoxybenzoyl)amino]-2-hydroxyphenyl}-1H- |
| | pyrazole-1-carboxamide |
| 329 | 5-cyclobutyl-3-{4-[(4-ethoxybenzoyl)amino]-2-hydroxyphenyl}-N-(2- |
| | phenylethyl)-1H-pyrazole-1-carboxamide |
| 330 | 5-cyclobutyl-3-{4-[(4-ethoxybenzoyl)amino]-2-hydroxyphenyl}-N-(2- |
| | methylbenzyl)-1H-pyrazole-1-carboxamide |
| 331 | 5-cyclobutyl-3-{4-[(4-ethoxybenzoyl)amino]-2-hydroxyphenyl}-N-(3- |
| | methylbenzyl)-1H-pyrazole-1-carboxamide |
| 332 | 5-cyclobutyl-3-{4-[(4-ethoxybenzoyl)amino]-2-hydroxyphenyl}-N-(4- |
| | methylbenzyl)-1H-pyrazole-1-carboxamide |
| 333 | N-(2-chlorobenzyl)-5-cyclobutyl-3-{4-[(4-ethoxybenzoyl)amino]-2- |
| | hydroxyphenyl}-1H-pyrazole-1-carboxamide |
| 334 | 5-cyclobutyl-3-{4-[(4-ethoxybenzoyl)amino]-2-hydroxyphenyl}-N-(4- |
| | fluorobenzyl)-1H-pyrazole-1-carboxamide |
| 335 | 5-cyclobutyl-N-(3,4-dichlorobenzyl)-3-{4-[(4-ethoxybenzoyl)amino]-2- |
| | hydroxyphenyl}-1H-pyrazole-1-carboxamide |
| 336 | 5-cyclobutyl-3-{4-[(4-ethoxybenzoyl)amino]-2-hydroxyphenyl}-N-(4- |
| | methoxybenzyl)-1H-pyrazole-1-carboxamide |
| 337 | N-(sec-butyl)-5-cyclobutyl-3-{4-[(4-ethoxybenzoyl)amino]-2-hydroxyphenyl}- |
| | 1H-pyrazole-1-carboxamide |
| 338 | 5-cyclobutyl-3-{4-[(4-ethoxybenzoyl)amino]-2-hydroxyphenyl}-N-(2- |
| | fluorobenzyl)-1H-pyrazole-1-carboxamide |
| 339 | 5-cyclobutyl-3-{4-[(4-ethoxybenzoyl)amino]-2-hydroxyphenyl}-N-(3- |
| | fluorobenzyl)-1H-pyrazole-1-carboxamide |
| 340 | 5-cyclobutyl-N-cyclopropyl-3-{4-[(4-ethoxybenzoyl)amino]-2-hydroxyphenyl}- |
| | 1H-pyrazole-1-carboxamide |
| 341 | 5-cyclopentyl-3-{4-[(4-ethoxybenzoyl)amino]-2-hydroxyphenyl}-N-isopropyl-1H- |
| | pyrazole-1-carboxamide |
| 342 | 5-cyclopentyl-3-{4-[(4-ethoxybenzoyl)amino]-2-hydroxyphenyl}-N-ethyl-1H- |
| | pyrazole-1-carboxamide |
| 343 | N-allyl-5-cyclopentyl-3-{4-[(4-ethoxybenzoyl)amino]-2-hydroxyphenyl}-1H- |
| | pyrazole-1-carboxamide |
| 344 | 5-cyclopentyl-3-{4-[(4-ethoxybenzoyl)amino]-2-hydroxyphenyl}-N-propyl-1H- |
| | pyrazole-1-carboxamide |
| 345 | N-benzyl-5-cyclopentyl-3-{4-[(4-ethoxybenzoyl)amino]-2-hydroxyphenyl}-1H- |
| | pyrazole-1-carboxamide |
| 346 | 5-cyclopentyl-3-{4-[(4-ethoxybenzoyl)amino]-2-hydroxyphenyl}-N-(2- |
| | phenylethyl)-1H-pyrazole-1-carboxamide |
| 347 | 5-cyclopentyl-3-{4-[(4-ethoxybenzoyl)amino]-2-hydroxyphenyl}-N-(2- |
| | methylbenzyl)-1H-pyrazole-1-carboxamide |
| 348 | 5-cyclopentyl-3-{4-[(4-ethoxybenzoyl)amino]-2-hydroxyphenyl}-N-(3- |
| | methylbenzyl)-1H-pyrazole-1-carboxamide |
| 349 | 5-cyclopentyl-3-{4-[(4-ethoxybenzoyl)amino]-2-hydroxyphenyl}-N-(4- |
| | methylbenzyl)-1H-pyrazole-1-carboxamide |
| 350 | N-(2-chlorobenzyl)-5-cyclopentyl-3-{4-[(4-ethoxybenzoyl)amino]-2- |
| | hydroxyphenyl}-1H-pyrazole-1-carboxamide |
| 351 | 5-cyclopentyl-3-{4-[(4-ethoxybenzoyl)amino]-2-hydroxyphenyl}-N-(4- |
| | fluorobenzyl)-1H-pyrazole-1-carboxamide |
| 352 | 5-cyclopentyl-N-(3,4-dichlorobenzyl)-3-{4-[(4-ethoxybenzoyl)amino]-2- |
| | hydroxyphenyl}-1H-pyrazole-1-carboxamide |
| 353 | 5-cyclopentyl-3-{4-[(4-ethoxybenzoyl)amino]-2-hydroxyphenyl}-N-(4- |
| | methoxybenzyl)-1H-pyrazole-1-carboxamide |
| 354 | N-(sec-butyl)-5-cyclopentyl-3-{4-[(4-ethoxybenzoyl)amino]-2-hydroxyphenyl}- |
| | 1H-pyrazole-1-carboxamide |
| 355 | 5-cyclopentyl-3-{4-[(4-ethoxybenzoyl)amino]-2-hydroxyphenyl}-N-(2- |
| | fluorobenzyl)-1H-pyrazole-1-carboxamide |
| 356 | 5-cyclopentyl-3-{4-[(4-ethoxybenzoyl)amino]-2-hydroxyphenyl}-N-(3- |
| | fluorobenzyl)-1H-pyrazole-1-carboxamide |
| 357 | 5-cyclopentyl-N-cyclopropyl-3-{4-[(4-ethoxybenzoyl)amino]-2-hydroxyphenyl}- |
| | 1H-pyrazole-1-carboxamide |
| 358 | 3-{4-[(4-tert-butylbenzoyl)amino]-2-hydroxyphenyl}-5-cyclopropyl-N-propyl-1H- |
| | pyrazole-1-carboxamide |
| 359 | 3-{4-[(4-tert-butylbenzoyl)amino]-2-hydroxyphenyl}-5-cyclopropyl-N-ethyl-1H- |
| | pyrazole-1-carboxamide |
| 360 | N-allyl-3-{4-[(4-tert-butylbenzoyl)amino]-2-hydroxyphenyl}-5-cyclopropyl-1H- |
| | pyrazole-1-carboxamide |
| 361 | 3-{4-[(4-tert-butylbenzoyl)amino]-2-hydroxyphenyl}-5-cyclopropyl-N-propyl-1H- |
| | pyrazole-1-carboxamide |
| 362 | N-benzyl-3-{4-[(4-tert-butylbenzoyl)amino]-2-hydroxyphenyl}-5-cyclopropyl-1H- |
| | pyrazole-1-carboxamide |
| 363 | 3-{4-[(4-tert-butylbenzoyl)amino]-2-hydroxyphenyl}-5-cyclopropyl-N-(2- |
| | phenylethyl)-1H-pyrazole-1-carboxamide |
| 364 | 3-{4-[(4-tert-butylbenzoyl)amino]-2-hydroxyphenyl}-5-cyclopropyl-N-(2- |
| | methylbenzyl)-1H-pyrazole-1-carboxamide |
| 365 | 3-{4-[(4-tert-butylbenzoyl)amino]-2-hydroxyphenyl}-5-cyclopropyl-N-(3- |
| | methylbenzyl)-1H-pyrazole-1-carboxamide |
| 366 | 3-{4-[(4-tert-butylbenzoyl)amino]-2-hydroxyphenyl}-5-cyclopropyl-N-(4- |
| | methylbenzyl)-1H-pyrazole-1-carboxamide |
| 367 | 3-{4-[(4-tert-butylbenzoyl)amino]-2-hydroxyphenyl}-N-(2-chlorobenzyl)-5- |
| | cyclopropyl-1H-pyrazole-1-carboxamide |
| 368 | 3-{4-[(4-tert-butylbenzoyl)amino]-2-hydroxyphenyl}-5-cyclopropyl-N-(4- |
| | fluorobenzyl)-1H-pyrazole-1-carboxamide |
| 369 | 3-{4-[(4-tert-butylbenzoyl)amino]-2-hydroxyphenyl}-5-cyclopropyl-N-(3,4- |
| | dichlorobenzyl)-1H-pyrazole-1-carboxamide |
| 370 | 3-{4-[(4-tert-butylbenzoyl)amino]-2-hydroxyphenyl}-5-cyclopropyl-N-(4- |
| | methoxybenzyl)-1H-pyrazole-1-carboxamide |
| 371 | N-(sec-butyl)-3-{4-[(4-tert-butylbenzoyl)amino]-2-hydroxyphenyl}-5- |
| | cyclopropyl-1H-pyrazole-1-carboxamide |
| 372 | 3-{4-[(4-tert-butylbenzoyl)amino]-2-hydroxyphenyl}-5-cyclopropyl-N-(2- |
| | fluorobenzyl)-1H-pyrazole-1-carboxamide |
| 373 | 3-{4-[(4-tert-butylbenzoyl)amino]-2-hydroxyphenyl}-5-cyclopropyl-N-(3- |
| | fluorobenzyl)-1H-pyrazole-1-carboxamide |
| 374 | 3-{4-[(4-tert-butylbenzoyl)amino]-2-hydroxyphenyl}-N,5-dicyclopropyl-1H- |
| | pyrazole-1-carboxamide |
| 375 | 3-{4-[(4-tert-butylbenzoyl)amino]-2-hydroxyphenyl}-5-cyclobutyl-N-isopropyl- |
| | 1H-pyrazole-1-carboxamide |
| 376 | 3-{4-[(4-tert-butylbenzoyl)amino]-2-hydroxyphenyl}-5-cyclobutyl-N-ethyl-1H- |
| | pyrazole-1-carboxamide |
| 377 | N-allyl-3-{4-[(4-tert-butylbenzoyl)amino]-2-hydroxyphenyl}-5-cyclobutyl-1H- |
| | pyrazole-1-carboxamide |
| 378 | 3-{4-[(4-tert-butylbenzoyl)amino]-2-hydroxyphenyl}-5-cyclobutyl-N-propyl-1H- |
| | pyrazole-1-carboxamide |
| 379 | N-benzyl-3-{4-[(4-tert-butylbenzoyl)amino]-2-hydroxyphenyl}-5-cyclobutyl-1H- |
| | pyrazole-1-carboxamide |
| 380 | 3-{4-[(4-tert-butylbenzoyl)amino]-2-hydroxyphenyl}-5-cyclobutyl-N-(2- |
| | phenylethyl)-1H-pyrazole-1-carboxamide |
| 381 | 3-{4-[(4-tert-butylbenzoyl)amino]-2-hydroxyphenyl}-5-cyclobutyl-N-(2- |
| | methylbenzyl)-1H-pyrazole-1-carboxamide |
| 382 | 3-{4-[(4-tert-butylbenzoyl)amino]-2-hydroxyphenyl}-5-cyclobutyl-N-(3- |
| | methylbenzyl)-1H-pyrazole-1-carboxamide |
| 383 | 3-{4-[(4-tert-butylbenzoyl)amino]-2-hydroxyphenyl}-5-cyclobutyl-N-(4- |
| | methylbenzyl)-1H-pyrazole-1-carboxamide |
| 384 | 3-{4-[(4-tert-butylbenzoyl)amino]-2-hydroxyphenyl}-N-(2-chlorobenzyl)-5- |
| | cyclobutyl-1H-pyrazole-1-carboxamide |
| 385 | 3-{4-[(4-tert-butylbenzoyl)amino]-2-hydroxyphenyl}-5-cyclobutyl-N-(4- |
| | fluorobenzyl)-1H-pyrazole-1-carboxamide |
| 386 | 3-{4-[(4-tert-butylbenzoyl)amino]-2-hydroxyphenyl}-5-cyclobutyl-N-(3,4- |
| | dichlorobenzyl)-1H-pyrazole-1-carboxamide |
| 387 | 3-{4-[(4-tert-butylbenzoyl)amino]-2-hydroxyphenyl}-5-cyclobutyl-N-(4- |
| | methoxybenzyl)-1H-pyrazole-1-carboxamide |
| 388 | N-(sec-butyl)-3-{4-[(4-tert-butylbenzoyl)amino]-2-hydroxyphenyl}-5-cyclobutyl- |
| | 1H-pyrazole-1-carboxamide |
| 389 | 3-{4-[(4-tert-butylbenzoyl)amino]-2-hydroxyphenyl}-5-cyclobutyl-N-(2- |
| | fluorobenzyl)-1H-pyrazole-1-carboxamide |
| 390 | 3-{4-[(4-tert-butylbenzoyl)amino]-2-hydroxyphenyl}-5-cyclobutyl-N-(3- |
| | fluorobenzyl)-1H-pyrazole-1-carboxamide |
| 391 | 3-{4-[(4-tert-butylbenzoyl)amino]-2-hydroxyphenyl}-5-cyclobutyl-N-cyclopropyl- |
| | 1H-pyrazole-1-carboxamide |
| 392 | 3-{4-[(4-tert-butylbenzoyl)amino]-2-hydroxyphenyl}-5-cyclopentyl-N-isopropyl- |
| | 1H-pyrazole-1-carboxamide |
| 393 | 3-{4-[(4-tert-butylbenzoyl)amino]-2-hydroxyphenyl}-5-cyclopentyl-N-ethyl-1H- |
| | pyrazole-1-carboxamide |
| 394 | N-allyl-3-{4-[(4-tert-butylbenzoyl)amino]-2-hydroxyphenyl}-5-cyclopentyl-1H- |
| | pyrazole-1-carboxamide |
| 395 | 3-{4-[(4-tert-butylbenzoyl)amino]-2-hydroxyphenyl}-5-cyclopentyl-N-propyl-1H- |
| | pyrazole-1-carboxamide |
| 396 | N-benzyl-3-{4-[(4-tert-butylbenzoyl)amino]-2-hydroxyphenyl}-5-cyclopentyl-1H- |
| | pyrazole-1-carboxamide |
| 397 | 3-{4-[(4-tert-butylbenzoyl)amino]-2-hydroxyphenyl}-5-cyclopentyl-N-(2- |
| | phenylethyl)-1H-pyrazole-1-carboxamide |
| 398 | 3-{4-[(4-tert-butylbenzoyl)amino]-2-hydroxyphenyl}-5-cyclopentyl-N-(2- |
| | methylbenzyl)-1H-pyrazole-1-carboxamide |
| 399 | 3-{4-[(4-tert-butylbenzoyl)amino]-2-hydroxyphenyl}-5-cyclopentyl-N-(3- |
| | methylbenzyl)-1H-pyrazole-1-carboxamide |
| 400 | 3-{4-[(4-tert-butylbenzoyl)amino]-2-hydroxyphenyl}-5-cyclopentyl-N-(4- |
| | methylbenzyl)-1H-pyrazole-1-carboxamide |
| 401 | 3-{4-[(4-tert-butylbenzoyl)amino]-2-hydroxyphenyl}-N-(2-chlorobenzyl)-5- |
| | cyclopentyl-1H-pyrazole-1-carboxamide |
| 402 | 3-{4-[(4-tert-butylbenzoyl)amino]-2-hydroxyphenyl}-5-cyclopentyl-N-(4- |
| | fluorobenzyl)-1H-pyrazole-1-carboxamide |
| 403 | 3-{4-[(4-tert-butylbenzoyl)amino]-2-hydroxyphenyl}-5-cyclopentyl-N-(3,4- |
| | dichlorobenzyl)-1H-pyrazole-1-carboxamide |
| 404 | 3-{4-[(4-tert-butylbenzoyl)amino]-2-hydroxyphenyl}-5-cyclopentyl-N-(4- |
| | methoxybenzyl)-1H-pyrazole-1-carboxamide |
| 405 | N-(sec-butyl)-3-{4-[(4-tert-butylbenzoyl)amino]-2-hydroxyphenyl}-5-cyclopentyl- |
| | 1H-pyrazole-1-carboxamide |
| 406 | 3-{4-[(4-tert-butylbenzoyl)amino]-2-hydroxyphenyl}-5-cyclopentyl-N-(2- |
| | fluorobenzyl)-1H-pyrazole-1-carboxamide |
| 407 | 3-{4-[(4-tert-butylbenzoyl)amino]-2-hydroxyphenyl}-5-cyclopentyl-N-(3- |
| | fluorobenzyl)-1H-pyrazole-1-carboxamide |
| 408 | 3-{4-[(4-tert-butylbenzoyl)amino]-2-hydroxyphenyl}-5-cyclopentyl-N- |
| | cyclopropyl-1H-pyrazole-1-carboxamide |
| 409 | 5-cyclopropyl-3-{2-hydroxy-4-[(4-methylbenzoyl)amino]phenyl}-N-isopropyl- |
| | 1H-pyrazole-1-carboxamide |
| 410 | 5-cyclopropyl-N-ethyl-3-{2-hydroxy-4-[(4-methylbenzoyl)amino]phenyl}-1H- |
| | pyrazole-1-carboxamide |
| 411 | N-allyl-5-cyclopropyl-3-{2-hydroxy-4-[(4-methylbenzoyl)amino]phenyl}-1H- |
| | pyrazole-1-carboxamide |
| 412 | 5-cyclopropyl-3-{2-hydroxy-4-[(4-methylbenzoyl)amino]phenyl}-N-propyl-1H- |
| | pyrazole-1-carboxamide |
| 413 | N-benzyl-5-cyclopropyl-3-{2-hydroxy-4-[(4-methylbenzoyl)amino]phenyl}-1H- |
| | pyrazole-1-carboxamide |
| 414 | 5-cyclopropyl-3-{2-hydroxy-4-[(4-methylbenzoyl)amino]phenyl}-N-(2- |
| | phenylethyl)-1H-pyrazole-1-carboxamide |
| 415 | 5-cyclopropyl-3-{2-hydroxy-4-[(4-methylbenzoyl)amino]phenyl}-N-(2- |
| | methylbenzyl)-1H-pyrazole-1-carboxamide |
| 416 | 5-cyclopropyl-3-{2-hydroxy-4-[(4-methylbenzoyl)amino]phenyl}-N-(3- |
| | methylbenzyl)-1H-pyrazole-1-carboxamide |
| 417 | 5-cyclopropyl-3-{2-hydroxy-4-[(4-methylbenzoyl)amino]phenyl}-N-(4- |
| | methylbenzyl)-1H-pyrazole-1-carboxamide |
| 418 | N-(2-chlorobenzyl)-5-cyclopropyl-3-{2-hydroxy-4-[(4- |
| | methylbenzoyl)amino]phenyl}-1H-pyrazole-1-carboxamide |
| 419 | 5-cyclopropyl-N-(4-fluorobenzyl)-3-{2-hydroxy-4-[(4- |
| | methylbenzoyl)amino]phenyl}-1H-pyrazole-1-carboxamide |
| 420 | 5-cyclopropyl-N-(3,4-dichlorobenzyl)-3-{2-hydroxy-4-[(4- |
| | methylbenzoyl)amino]phenyl}-1H-pyrazole-1-carboxamide |
| 421 | 5-cyclopropyl-3-{2-hydroxy-4-[(4-methylbenzoyl)amino]phenyl}-N-(4- |
| | methoxybenzyl)-1H-pyrazole-1-carboxamide |
| 422 | N-(sec-butyl)-5-cyclopropyl-3-{2-hydroxy-4-[(4-methylbenzoyl)amino]phenyl}- |
| | 1H-pyrazole-1-carboxamide |
| 423 | 5-cyclopropyl-N-(2-fluorobenzyl)-3-{2-hydroxy-4-[(4- |
| | methylbenzoyl)amino]phenyl}-1H-pyrazole-1-carboxamide |
| 424 | 5-cyclopropyl-N-(3-fluorobenzyl)-3-{2-hydroxy-4-[(4- |
| | methylbenzoyl)amino]phenyl}-1H-pyrazole-1-carboxamide |
| 425 | N,5-dicyclopropyl-3-{2-hydroxy-4-[(4-methylbenzoyl)amino]phenyl}-1H- |
| | pyrazole-1-carboxamide |
| 426 | 5-cyclobutyl-3-{2-hydroxy-4-[(4-methylbenzoyl)amino]phenyl}-N-isopropyl-1H- |
| | pyrazole-1-carboxamide |
| 427 | 5-cyclobutyl-N-ethyl-3-{2-hydroxy-4-[(4-methylbenzoyl)amino]phenyl}-1H- |
| | pyrazole-1-carboxamide |
| 428 | N-allyl-5-cyclobutyl-3-{2-hydroxy-4-[(4-methylbenzoyl)amino]phenyl}-1H- |
| | pyrazole-1-carboxamide |
| 429 | 5-cyclobutyl-3-{2-hydroxy-4-[(4-methylbenzoyl)amino]phenyl}-N-propyl-1H- |
| | pyrazole-1-carboxamide |
| 430 | N-benzyl-5-cyclobutyl-3-{2-hydroxy-4-[(4-methylbenzoyl)amino]phenyl}-1H- |
| | pyrazole-1-carboxamide |
| 431 | 5-cyclobutyl-3-{2-hydroxy-4-[(4-methylbenzoyl)amino]phenyl}-N-(2- |
| | phenylethyl)-1H-pyrazole-1-carboxamide |
| 432 | 5-cyclobutyl-3-{2-hydroxy-4-[(4-methylbenzoyl)amino]phenyl}-N-(2- |
| | methylbenzyl)-1H-pyrazole-1-carboxamide |
| 433 | 5-cyclobutyl-3-{2-hydroxy-4-[(4-methylbenzoyl)amino]phenyl}-N-(3- |
| | methylbenzyl)-1H-pyrazole-1-carboxamide |
| 434 | 5-cyclobutyl-3-{2-hydroxy-4-[(4-methylbenzoyl)amino]phenyl}-N-(4- |
| | methylbenzyl)-1H-pyrazole-1-carboxamide |
| 435 | N-(2-chlorobenzyl)-5-cyclobutyl-3-{2-hydroxy-4-[(4- |
| | methylbenzoyl)amino]phenyl}-1H-pyrazole-1-carboxamide |
| 436 | 5-cyclobutyl-N-(4-fluorobenzyl)-3-{2-hydroxy-4-[(4- |
| | methylbenzoyl)amino]phenyl}-1H-pyrazole-1-carboxamide |
| 437 | 5-cyclobutyl-N-(3,4-dichlorobenzyl)-3-{2-hydroxy-4-[(4- |
| | methylbenzoyl)amino]phenyl}-1H-pyrazole-1-carboxamide |
| 438 | 5-cyclobutyl-3-{2-hydroxy-4-[(4-methylbenzoyl)amino]phenyl}-N-(4- |
| | methoxybenzyl)-1H-pyrazole-1-carboxamide |
| 439 | N-(sec-butyl)-5-cyclobutyl-3-{2-hydroxy-4-[(4-methylbenzoyl)amino]phenyl}- |
| | 1H-pyrazole-1-carboxamide |
| 440 | 5-cyclobutyl-N-(2-fluorobenzyl)-3-{2-hydroxy-4-[(4- |
| | methylbenzoyl)amino]phenyl}-1H-pyrazole-1-carboxamide |
| 441 | 5-cyclopentyl-3-{2-hydroxy-4-[(4-methylbenzoyl)amino]phenyl}-N-propyl-1H- |
| | pyrazole-1-carboxamide |
| 442 | 5-cyclobutyl-N-cyclopropyl-3-{2-hydroxy-4-[(4-methylbenzoyl)amino]phenyl}- |
| | 1H-pyrazole-1-carboxamide |
| 443 | 5-cyclopentyl-3-{2-hydroxy-4-[(4-methylbenzoyl)amino]phenyl}-N-isopropyl- |
| | 1H-pyrazole-1-carboxamide |
| 444 | 5-cyclopentyl-N-ethyl-3-{2-hydroxy-4-[(4-methylbenzoyl)amino]phenyl}-1H- |
| | pyrazole-1-carboxamide |
| 445 | N-allyl-5-cyclopentyl-3-{2-hydroxy-4-[(4-methylbenzoyl)amino]phenyl}-1H- |
| | pyrazole-1-carboxamide |
| 446 | 5 -cyclopentyl-3-{2-hydroxy-4-[(4-methylbenzoyl)amino]phenyl}-N-propyl-1H- |
| | pyrazole-1-carboxamide |
| 447 | N-benzyl-5-cyclopentyl-3-{2-hydroxy-4-[(4-methylbenzoyl)amino]phenyl}-1H- |
| | pyrazole-1-carboxamide |
| 448 | 5-cyclopentyl-3-{2-hydroxy-4-[(4-methylbenzoyl)amino]phenyl}-N-(2- |
| | phenylethyl)-1H-pyrazole-1-carboxamide |
| 449 | 5-cyclopentyl-3-{2-hydroxy-4-[(4-methylbenzoyl)amino]phenyl}-N-(2- |
| | methylbenzyl)-1H-pyrazole-1-carboxamide |
| 450 | 5-cyclopentyl-3-{2-hydroxy-4-[(4-methylbenzoyl)amino]phenyl}-N-(3- |
| | methylbenzyl)-1H-pyrazole-1-carboxamide |
| 451 | 5-cyclopentyl-3-{2-hydroxy-4-[(4-methylbenzoyl)amino]phenyl}-N-(4- |
| | methylbenzyl)-1H-pyrazole-1-carboxamide |
| 452 | N-(2-chlorobenzyl)-5-cyclopentyl-3-{2-hydroxy-4-[(4- |
| | methylbenzoyl)amino]phenyl}-1H-pyrazole-1-carboxamide |
| 453 | 5-cyclopentyl-N-(4-fluorobenzyl)-3-{2-hydroxy-4-[(4- |
| | methylbenzoyl)amino]phenyl}-1H-pyrazole-1-carboxamide |
| 454 | 5-cyclopentyl-N-(3,4-dichlorobenzyl)-3-{2-hydroxy-4-[(4- |
| | methylbenzoyl)amino]phenyl}-1H-pyrazole-1-carboxamide |
| 455 | 5-cyclopentyl-3-{2-hydroxy-4-[(4-methylbenzoyl)amino]phenyl}-N-(4- |
| | methoxybenzyl)-1H-pyrazole-1-carboxamide |
| 456 | N-(sec-butyl)-5-cyclopentyl-3-{2-hydroxy-4-[(4-methylbenzoyl)amino]phenyl}- |
| | 1H-pyrazole-1-carboxamide |
| 457 | 5-cyclopentyl-N-(2-fluorobenzyl)-3-{2-hydroxy-4-[(4- |
| | methylbenzoyl)amino]phenyl}-1H-pyrazole-1-carboxamide |
| 458 | 5-cyclopentyl-N-(3-fluorobenzyl)-3-{2-hydroxy-4-[(4- |
| | methylbenzoyl)amino]phenyl}-1H-pyrazole-1-carboxamide |
| 459 | 5-cyclopentyl-N-cyclopropyl-3-{2-hydroxy-4-[(4-methylbenzoyl)amino]phenyl}- |
| | 1H-pyrazole-1-carboxamide |
| 460 | 5-cyclopropyl-3-{4-[(4-ethylbenzoyl)amino]-2-hydroxyphenyl}-N-isopropyl-1H- |
| | pyrazole-1-carboxamide |
| 461 | 5-cyclopropyl-N-ethyl-3-{4-[(4-ethylbenzoyl)amino]-2-hydroxyphenyl}-1H- |
| | pyrazole-1-carboxamide |
| 462 | N-allyl-5-cyclopropyl-3-{4-[(4-ethylbenzoyl)amino]-2-hydroxyphenyl}-1H- |
| | pyrazole-1-carboxamide |
| 463 | 5-cyclopropyl-3-{4-[(4-ethylbenzoyl)amino]-2-hydroxyphenyl}-N-propyl-1H- |
| | pyrazole-1-carboxamide |
| 464 | N-benzyl-5-cyclopropyl-3-{4-[(4-ethylbenzoyl)amino]-2-hydroxyphenyl}-1H- |
| | pyrazole-1-carboxamide |
| 465 | 5-cyclopropyl-3-{4-[(4-ethylbenzoyl)amino]-2-hydroxyphenyl}-N-(2- |
| | phenylethyl)-1H-pyrazole-1-carboxamide |
| 466 | 5-cyclopropyl-3-{4-[(4-ethylbenzoyl)amino]-2-hydroxyphenyl}-N-(2- |
| | methylbenzyl)-1H-pyrazole-1-carboxamide |
| 467 | 5-cyclopropyl-3-{4-[(4-ethylbenzoyl)amino]-2-hydroxyphenyl}-N-(3- |
| | methylbenzyl)-1H-pyrazole-1-carboxamide |
| 468 | 5-cyclobutyl-3-{4-[(4-ethylbenzoyl)amino]-2-hydroxyphenyl}-N-isopropyl-1H- |
| | pyrazole-1-carboxamide |
| 469 | N-(2-chlorobenzyl)-5-cyclopropyl-3-{4-[(4-ethylbenzoyl)amino]-2- |
| | hydroxyphenyl}-1H-pyrazole-1-carboxamide |
| 470 | 5-cyclopropyl-3-{4-[(4-ethylbenzoyl)amino]-2-hydroxyphenyl}-N-(4- |
| | fluorobenzyl)-1H-pyrazole-1-carboxamide |
| 471 | 5-cyclopropyl-N-(3,4-dichlorobenzyl)-3-{4-[(4-ethylbenzoyl)amino]-2- |
| | hydroxyphenyl}-1H-pyrazole-1-carboxamide |
| 472 | 5-cyclopropyl-3-{4-[(4-ethylbenzoyl)amino]-2-hydroxyphenyl}-N-(4- |
| | methoxybenzyl)-1H-pyrazole-1-carboxamide |
| 473 | N-(sec-butyl)-5-cyclopropyl-3-{4-[(4-ethylbenzoyl)amino]-2-hydroxyphenyl}-1H- |
| | pyrazole-1-carboxamide |
| 474 | 5-cyclopropyl-3-{4-[(4-ethylbenzoyl)amino]-2-hydroxyphenyl}-N-(2- |
| | fluorobenzyl)-1H-pyrazole-1-carboxamide |
| 475 | 5-cyclopropyl-3-{4-[(4-ethylbenzoyl)amino]-2-hydroxyphenyl}-N-(3- |
| | fluorobenzyl)-1H-pyrazole-1-carboxamide |
| 476 | N,5-dicyclopropyl-3-{4-[(4-ethylbenzoyl)amino]-2-hydroxyphenyl}-1H-pyrazole- |
| | 1-carboxamide |
| 477 | 5-cyclobutyl-3-{4-[(4-ethylbenzoyl)amino]-2-hydroxyphenyl}-N-isopropyl-1H- |
| | pyrazole-1-carboxamide |
| 478 | 5-cyclobutyl-N-ethyl-3-{4-[(4-ethylbenzoyl)amino]-2-hydroxyphenyl}-1H- |
| | pyrazole-1-carboxamide |
| 479 | N-allyl-5-cyclobutyl-3-{4-[(4-ethylbenzoyl)amino]-2-hydroxyphenyl}-1H- |
| | pyrazole-1-carboxamide |
| 480 | 5-cyclobutyl-3-{4-[(4-ethylbenzoyl)amino]-2-hydroxyphenyl}-N-propyl-1H- |
| | pyrazole-1-carboxamide |
| 481 | N-benzyl-5-cyclobutyl-3-{4-[(4-ethylbenzoyl)amino]-2-hydroxyphenyl}-1H- |
| | pyrazole-1-carboxamide |
| 482 | 5-cyclobutyl-3-{4-[(4-ethylbenzoyl)amino]-2-hydroxyphenyl}-N-(2-phenylethyl)- |
| | 1H-pyrazole-1-carboxamide |
| 483 | 5-cyclobutyl-3-{4-[(4-ethylbenzoyl)amino]-2-hydroxyphenyl}-N-(2- |
| | methylbenzyl)-1H-pyrazole-1-carboxamide |
| 484 | 5-cyclobutyl-3-{4-[(4-ethylbenzoyl)amino]-2-hydroxyphenyl}-N-(3- |
| | methylbenzyl)-1H-pyrazole-1-carboxamide |
| 485 | 5-cyclobutyl-3-{4-[(4-ethylbenzoyl)amino]-2-hydroxyphenyl}-N-(4- |
| | methylbenzyl)-1H-pyrazole-1-carboxamide |
| 486 | N-(2-chlorobenzyl)-5-cyclobutyl-3-{4-[(4-ethylbenzoyl)amino]-2- |
| | hydroxyphenyl}-1H-pyrazole-1-carboxamide |
| 487 | 5-cyclobutyl-3-{4-[(4-ethylbenzoyl)amino]-2-hydroxyphenyl}-N-(4- |
| | fluorobenzyl)-1H-pyrazole-1-carboxamide |
| 488 | 5-cyclobutyl-N-(3,4-dichlorobenzyl)-3-{4-[(4-ethylbenzoyl)amino]-2- |
| | hydroxyphenyl}-1H-pyrazole-1-carboxamide |
| 489 | 5-cyclobutyl-3-{4-[(4-ethylbenzoyl)amino]-2-hydroxyphenyl}-N-(4- |
| | methoxybenzyl)-1H-pyrazole-1-carboxamide |
| 490 | N-(sec-butyl)-5-cyclobutyl-3-{4-[(4-ethylbenzoyl)amino]-2-hydroxyphenyl}-1H- |
| | pyrazole-1-carboxamide |
| 491 | 5-cyclobutyl-3-{4-[(4-ethylbenzoyl)amino]-2-hydroxyphenyl}-N-(2- |
| | fluorobenzyl)-1H-pyrazole-1-carboxamide |
| 492 | 5-cyclobutyl-3-{4-[(4-ethylbenzoyl)amino]-2-hydroxyphenyl}-N-(3- |
| | fluorobenzyl)-1H-pyrazole-1-carboxamide |
| 493 | 5-cyclobutyl-N-cyclopropyl-3-{4-[(4-ethylbenzoyl)amino]-2-hydroxyphenyl}-1H- |
| | pyrazole-1-carboxamide |
| 494 | 5-cyclopentyl-3-{4-[(4-ethylbenzoyl)amino]-2-hydroxyphenyl}-N-isopropyl-1H- |
| | pyrazole-1-carboxamide |
| 495 | 5-cyclopentyl-N-ethyl-3-{4-[(4-ethylbenzoyl)amino]-2-hydroxyphenyl}-1H- |
| | pyrazole-1-carboxamide |
| 496 | N-allyl-5-cyclopentyl-3-{4-[(4-ethylbenzoyl)amino]-2-hydroxyphenyl}-1H- |
| | pyrazole-1-carboxamide |
| 497 | 5-cyclopentyl-3-{4-[(4-ethylbenzoyl)amino]-2-hydroxyphenyl}-N-propyl-1H- |
| | pyrazole-1-carboxamide |
| 498 | N-benzyl-5 -cyclopentyl-3-{4-[(4-ethylbenzoyl)amino]-2-hydroxyphenyl}-1H- |
| | pyrazole-1-carboxamide |
| 499 | 5-cyclopentyl-3-{4-[(4-ethylbenzoyl)amino]-2-hydroxyphenyl}-N-(2- |
| | phenylethyl)-1H-pyrazole-1-carboxamide |
| 500 | 5-cyclopentyl-3-{4-[(4-ethylbenzoyl)amino]-2-hydroxyphenyl}-N-(2- |
| | methylbenzyl)-1H-pyrazole-1-carboxamide |
| 501 | 5-cyclopentyl-3-{4-[(4-ethylbenzoyl)amino]-2-hydroxyphenyl}-N-(3- |
| | methylbenzyl)-1H-pyrazole-1-carboxamide |
| 502 | 5-cyclopentyl-3-{4-[(4-ethylbenzoyl)amino]-2-hydroxyphenyl}-N-(4- |
| | methylbenzyl)-1H-pyrazole-1-carboxamide |
| 503 | N-(2-chlorobenzyl)-5-cyclopentyl-3-{4-[(4-ethylbenzoyl)amino]-2- |
| | hydroxyphenyl}-1H-pyrazole-1-carboxamide |
| 504 | 5-cyclopentyl-3-{4-[(4-ethylbenzoyl)amino]-2-hydroxyphenyl}-N-(4- |
| | fluorobenzyl)-1H-pyrazole-1-carboxamide |
| 505 | 5-cyclopentyl-N-(3,4-dichlorobenzyl)-3-{4-[(4-ethylbenzoyl)amino]-2- |
| | hydroxyphenyl}-1H-pyrazole-1-carboxamide |
| 506 | 5-cyclopentyl-3-{4-[(4-ethylbenzoyl)amino]-2-hydroxyphenyl}-N-(4- |
| | methoxybenzyl)-1H-pyrazole-1-carboxamide |
| 507 | N-(sec-butyl)-5-cyclopentyl-3-{4-[(4-ethylbenzoyl)amino]-2-hydroxyphenyl}-1H- |
| | pyrazole-1-carboxamide |
| 508 | 5-cyclopentyl-3-{4-[(4-ethylbenzoyl)amino]-2-hydroxyphenyl}-N-(2- |
| | fluorobenzyl)-1H-pyrazole-1-carboxamide |
| 509 | 5-cyclopentyl-3-{4-[(4-ethylbenzoyl)amino]-2-hydroxyphenyl}-N-(3- |
| | fluorobenzyl)-1H-pyrazole-1-carboxamide |
| 510 | 5-cyclopentyl-N-cyclopropyl-3-{4-[(4-ethylbenzoyl)amino]-2-hydroxyphenyl}- |
| | 1H-pyrazole-1-carboxamide |
| 511 | 5-cyclopropyl-3-[2-hydroxy-4-(isobutyrylamino)phenyl]-N-isopropyl-1H- |
| | pyrazole-1-carboxamide |
| 512 | 5-cyclopropyl-N-ethyl-3-[2-hydroxy-4-(isobutyrylamino)phenyl]-1H-pyrazole-1- |
| | carboxamide |
| 513 | N-allyl-5-cyclopropyl-3-[2-hydroxy-4-(isobutyrylamino)phenyl]-1H-pyrazole-1- |
| | carboxamide |
| 514 | 5-cyclopropyl-3-[2-hydroxy-4-(isobutyrylamino)phenyl]-N-propyl-1H-pyrazole-1- |
| | carboxamide |
| 515 | N-benzyl-5-cyclopropyl-3-[2-hydroxy-4-(isobutyrylamino)phenyl]-1H-pyrazole-1- |
| | carboxamide |
| 516 | 5-cyclopropyl-3-[2-hydroxy-4-(isobutyrylamino)phenyl]-N-(2-phenylethyl)-1H- |
| | pyrazole-1-carboxamide |
| 517 | 5-cyclopropyl-3-[2-hydroxy-4-(isobutyrylamino)phenyl]-N-(2-methylbenzyl)-1H- |
| | pyrazole-1-carboxamide |
| 518 | 5-cyclopropyl-3-[2-hydroxy-4-(isobutyrylamino)phenyl]-N-(3-methylbenzyl)-1H- |
| | pyrazole-1-carboxamide |
| 519 | 5-cyclopropyl-3-[2-hydroxy-4-(isobutyrylamino)phenyl]-N-(4-methylbenzyl)-1H- |
| | pyrazole-1-carboxamide |
| 520 | N-(2-chlorobenzyl)-5-cyclopropyl-3-[2-hydroxy-4-(isobutyrylamino)phenyl]-1H- |
| | pyrazole-1-carboxamide |
| 521 | 5-cyclopropyl-N-(4-fluorobenzyl)-3-[2-hydroxy-4-(isobutyrylamino)phenyl]-1H- |
| | pyrazole-1-carboxamide |
| 522 | 5-cyclopropyl-N-(3,4-dichlorobenzyl)-3-[2-hydroxy-4-(isobutyrylamino)phenyl]- |
| | 1H-pyrazole-1-carboxamide |
| 523 | 5-cyclopropyl-3-[2-hydroxy-4-(isobutyrylamino)phenyl]-N-(4-methoxybenzyl)- |
| | 1H-pyrazole-1-carboxamide |
| 524 | N-(sec-butyl)-5-cyclopropyl-3-[2-hydroxy-4-(isobutyrylamino)phenyl]-1H- |
| | pyrazole-1-carboxamide |
| 525 | 5-cyclopropyl-N-(2-fluorobenzyl)-3-[2-hydroxy-4-(isobutyrylamino)phenyl]-1H- |
| | pyrazole-1-carboxamide |
| 526 | 5-cyclopropyl-N-(3-fluorobenzyl)-3-[2-hydroxy-4-(isobutyrylamino)phenyl]-1H- |
| | pyrazole-1-carboxamide |
| 527 | N,5-dicyclopropyl-3-[2-hydroxy-4-(isobutyrylamino)phenyl]-1H-pyrazole-1- |
| | carboxamide |
| 528 | 5-cyclobutyl-3-[2-hydroxy-4-(isobutyrylamino)phenyl]-N-isopropyl-1H-pyrazole- |
| | 1-carboxamide |
| 529 | 5-cyclobutyl-N-ethyl-3-[2-hydroxy-4-(isobutyrylamino)phenyl]-1H-pyrazole-1- |
| | carboxamide |
| 530 | N-allyl-5-cyclobutyl-3-[2-hydroxy-4-(isobutyrylamino)phenyl]-1H-pyrazole-1- |
| | carboxamide |
| 531 | 5-cyclobutyl-3-[2-hydroxy-4-(isobutyrylamino)phenyl]-N-propyl-1H-pyrazole-1- |
| | carboxamide |
| 532 | N-benzyl-5-cyclobutyl-3-[2-hydroxy-4-(isobutyrylamino)phenyl]-1H-pyrazole-1- |
| | carboxamide |
| 533 | 5-cyclobutyl-3-[2-hydroxy-4-(isobutyrylamino)phenyl]-N-(2-phenylethyl)-1H- |
| | pyrazole-1-carboxamide |
| 534 | 5-cyclobutyl-3-[2-hydroxy-4-(isobutyrylamino)phenyl]-N-(2-methylbenzyl)-1H- |
| | pyrazole-1-carboxamide |
| 535 | 5-cyclobutyl-3-[2-hydroxy-4-(isobutyrylamino)phenyl]-N-(3-methylbenzyl)-1H- |
| | pyrazole-1-carboxamide |
| 536 | 5-cyclobutyl-3-[2-hydroxy-4-(isobutyrylamino)phenyl]-N-(4-methylbenzyl)-1H- |
| | pyrazole-1-carboxamide |
| 537 | N-(2-chlorobenzyl)-5-cyclobutyl-3-[2-hydroxy-4-(isobutyrylamino)phenyl]-1H- |
| | pyrazole-1-carboxamide |
| 538 | 5-cyclobutyl-N-(4-fluorobenzyl)-3-[2-hydroxy-4-(isobutyrylamino)phenyl]-1H- |
| | pyrazole-1-carboxamide |
| 539 | 5-cyclobutyl-N-(3,4-dichlorobenzyl)-3-[2-hydroxy-4-(isobutyrylamino)phenyl]- |
| | 1H-pyrazole-1-carboxamide |
| 540 | 5-cyclobutyl-3-[2-hydroxy-4-(isobutyrylamino)phenyl]-N-(4-methoxybenzyl)-1H- |
| | pyrazole-1-carboxamide |
| 541 | N-(sec-butyl)-5-cyclobutyl-3-[2-hydroxy-4-(isobutyrylamino)phenyl]-1H- |
| | pyrazole-1-carboxamide |
| 542 | 5-cyclobutyl-N-(2-fluorobenzyl)-3-[2-hydroxy-4-(isobutyrylamino)phenyl]-1H- |
| | pyrazole-1-carboxamide |
| 543 | 5-cyclobutyl-N-(3-fluorobenzyl)-3-[2-hydroxy-4-(isobutyrylamino)phenyl]-1H- |
| | pyrazole-1-carboxamide |
| 544 | 5-cyclobutyl-N-cyclopropyl-3-[2-hydroxy-4-(isobutyrylamino)phenyl]-1H- |
| | pyrazole-1-carboxamide |
| 545 | 5-cyclopentyl-3-[2-hydroxy-4-(isobutyrylamino)phenyl]-N-isopropyl-1H- |
| | pyrazole-1-carboxamide |
| 546 | 5-cyclopentyl-N-ethyl-3-[2-hydroxy-4-(isobutyrylamino)phenyl]-1H-pyrazole-1- |
| | carboxamide |
| 547 | N-allyl-5-cyclopentyl-3-[2-hydroxy-4-(isobutyrylamino)phenyl]-1H-pyrazole-1- |
| | carboxamide |
| 548 | 5-cyclopentyl-3-[2-hydroxy-4-(isobutyrylamino)phenyl]-N-propyl-1H-pyrazole-1- |
| | carboxamide |
| 549 | N-benzyl-5-cyclopentyl-3-[2-hydroxy-4-(isobutyrylamino)phenyl]-1H-pyrazole-1- |
| | carboxamide |
| 550 | 5-cyclopentyl-3-[2-hydroxy-4-(isobutyrylamino)phenyl]-N-(2-phenylethyl)-1H- |
| | pyrazole-1-carboxamide |
| 551 | 5-cyclopentyl-3-[2-hydroxy-4-(isobutyrylamino)phenyl]-N-(2-methylbenzyl)-1H- |
| | pyrazole-1-carboxamide |
| 552 | 5-cyclopentyl-3-[2-hydroxy-4-(isobutyrylamino)phenyl]-N-(3-methylbenzyl)-1H- |
| | pyrazole-1-carboxamide |
| 553 | 5-cyclopentyl-3-[2-hydroxy-4-(isobutyrylamino)phenyl]-N-(4-methylbenzyl)-1H- |
| | pyrazole-1-carboxamide |
| 554 | N-(2-chlorobenzyl)-5-cyclopentyl-3-[2-hydroxy-4-(isobutyrylamino)phenyl]-1H- |
| | pyrazole-1-carboxamide |
| 555 | 5-cyclopentyl-N-(4-fluorobenzyl)-3-[2-hydroxy-4-(isobutyrylamino)phenyl]-1H- |
| | pyrazole-1-carboxamide |
| 556 | 5-cyclopentyl-N-(3,4-dichlorobenzyl)-3-[2-hydroxy-4-(isobutyrylamino)phenyl]- |
| | 1H-pyrazole-1-carboxamide |
| 557 | 5-cyclopentyl-3-[2-hydroxy-4-(isobutyrylamino)phenyl]-N-(4-methoxybenzyl)- |
| | 1H-pyrazole-1-carboxamide |
| 558 | N-(sec-butyl)-5-cyclopentyl-3-[2-hydroxy-4-(isobutyrylamino)phenyl]-1H- |
| | pyrazole-1-carboxamide |
| 559 | 5-cyclopentyl-N-(2-fluorobenzyl)-3-[2-hydroxy-4-(isobutyrylamino)phenyl]-1H- |
| | pyrazole-1-carboxamide |
| 560 | 5-cyclopentyl-N-(3-fluorobenzyl)-3-[2-hydroxy-4-(isobutyrylamino)phenyl]-1H- |
| | pyrazole-1-carboxamide |
| 561 | 5-cyclopentyl-N-cyclopropyl-3-[2-hydroxy-4-(isobutyrylamino)phenyl]-1H- |
| | pyrazole-1-carboxamide |
| 562 | 3-[4-(acetylamino)-2-hydroxyphenyl]-5-cyclopropyl-N-isopropyl-1H-pyrazole-1- |
| | carboxamide |
| 563 | 3-[4-(acetylamino)-2-hydroxyphenyl]-5-cyclopropyl-N-ethyl-1H-pyrazole-1- |
| | carboxamide |
| 564 | 3-[4-(acetylamino)-2-hydroxyphenyl]-N-allyl-5-cyclopropyl-1H-pyrazole-1- |
| | carboxamide |
| 565 | 3-[4-(acetylamino)-2-hydroxyphenyl]-5-cyclopropyl-N-propyl-1H-pyrazole-1- |
| | carboxamide |
| 566 | 3-[4-(acetylamino)-2-hydroxyphenyl]-5-cyclopropyl-N-(2-phenylethyl)-1H- |
| | pyrazole-1-carboxamide |
| 567 | 3-[4-(acetylamino)-2-hydroxyphenyl]-5-cyclopropyl-N-(2-phenylethyl)-1H- |
| | pyrazole-1-carboxamide |
| 568 | 3-[4-(acetylamino)-2-hydroxyphenyl]-5-cyclopropyl-N-(2-methylbenzyl)-1H- |
| | pyrazole-1-carboxamide |
| 569 | 3-[4-(acetylamino)-2-hydroxyphenyl]-5-cyclopropyl-N-(3-methylbenzyl)-1H- |
| | pyrazole-1-carboxamide |
| 570 | 3-[4-(acetylamino)-2-hydroxyphenyl]-5-cyclopropyl-N-(4-methylbenzyl)-1H- |
| | pyrazole-1-carboxamide |
| 571 | 3-[4-(acetylamino)-2-hydroxyphenyl]-N-(2-chlorobenzyl)-5-cyclopropyl-1H- |
| | pyrazole-1-carboxamide |
| 572 | 3-[4-(acetylamino)-2-hydroxyphenyl]-5-cyclopropyl-N-(4-fluorobenzyl)-1H- |
| | pyrazole-1-carboxamide |
| 573 | 3-[4-(acetylamino)-2-hydroxyphenyl]-5-cyclopropyl-N-(3,4-dichlorobenzyl)-1H- |
| | pyrazole-1-carboxamide |
| 574 | 3-[4-(acetylamino)-2-hydroxyphenyl]-5-cyclopropyl-N-(4-methoxybenzyl)-1H- |
| | pyrazole-1-carboxamide |
| 575 | 3-[4-(acetylamino)-2-hydroxyphenyl]-N-(sec-butyl)-5-cyclopropyl-1H-pyrazole-1- |
| | carboxamide |
| 576 | 3-[4-(acetylamino)-2-hydroxyphenyl]-5-cyclopropyl-N-(2-fluorobenzyl)-1H- |
| | pyrazole-1-carboxamide |
| 577 | 3-[4-(acetylamino)-2-hydroxyphenyl]-5-cyclopropyl-N-(3-fluorobenzyl)-1H- |
| | pyrazole-1-carboxamide |
| 578 | 3-[4-(acetylamino)-2-hydroxyphenyl]-N,5-dicyclopropyl-1H-pyrazole-1- |
| | carboxamide |
| 579 | 3-[4-(acetylamino)-2-hydroxyphenyl]-5-cyclobutyl-N-isopropyl-1H-pyrazole-1- |
| | carboxamide |
| 580 | 3-[4-(acetylamino)-2-hydroxyphenyl]-5-cyclobutyl-N-ethyl-1H-pyrazole-1- |
| | carboxamide |
| 581 | 3-[4-(acetylamino)-2-hydroxyphenyl]-N-allyl-5-cyclobutyl-1H-pyrazole-1- |
| | carboxamide |
| 582 | 3-[4-(acetylamino)-2-hydroxyphenyl]-5-cyclobutyl-N-propyl-1H-pyrazole-1- |
| | carboxamide |
| 583 | 3-[4-(acetylamino)-2-hydroxyphenyl]-N-benzyl-5-cyclobutyl-1H-pyrazole-1- |
| | carboxamide |
| 584 | 3-[4-(acetylamino)-2-hydroxyphenyl]-5-cyclobutyl-N-(2-phenylethyl)-1H- |
| | pyrazole-1-carboxamide |
| 585 | 3-[4-(acetylamino)-2-hydroxyphenyl]-5-cyclobutyl-N-(2-methylbenzyl)-1H- |
| | pyrazole-1-carboxamide |
| 586 | 3-[4-(acetylamino)-2-hydroxyphenyl]-5-cyclobutyl-N-(3-methylbenzyl)-1H- |
| | pyrazole-1-carboxamide |
| 587 | 3-[4-(acetylamino)-2-hydroxyphenyl]-5-cyclobutyl-N-(4-methylbenzyl)-1H- |
| | pyrazole-1-carboxamide |
| 588 | 3-[4-(acetylamino)-2-hydroxyphenyl]-N-(2-chlorobenzyl)-5-cyclobutyl-1H- |
| | pyrazole-1-carboxamide |
| 589 | 3-[4-(acetylamino)-2-hydroxyphenyl]-5-cyclobutyl-N-(4-fluorobenzyl)-1H- |
| | pyrazole-1-carboxamide |
| 590 | 3-[4-(acetylamino)-2-hydroxyphenyl]-5-cyclobutyl-N-(3,4-dichlorobenzyl)-1H- |
| | pyrazole-1-carboxamide |
| 591 | 3-[4-(acetylamino)-2-hydroxyphenyl]-5-cyclobutyl-N-(4-methoxybenzyl)-1H- |
| | pyrazole-1-carboxamide |
| 592 | 3-[4-(acetylamino)-2-hydroxyphenyl]-N-(sec-butyl)-5-cyclobutyl-1H-pyrazole-1- |
| | carboxamide |
| 593 | 3-[4-(acetylamino)-2-hydroxyphenyl]-5-cyclobutyl-N-(2-fluorobenzyl)-1H- |
| | pyrazole-1-carboxamide |
| 594 | 3-[4-(acetylamino)-2-hydroxyphenyl]-5-cyclobutyl-N-(3-fluorobenzyl)-1H- |
| | pyrazole-1-carboxamide |
| 595 | 3-[4-(acetylamino)-2-hydroxyphenyl]-5-cyclobutyl-N-cyclopropyl-1H-pyrazole-1- |
| | carboxamide |
| 596 | 3-[4-(acetylamino)-2-hydroxyphenyl]-5-cyclopentyl-N-isopropyl-1H-pyrazole-1- |
| | carboxamide |
| 597 | 3-[4-(acetylamino)-2-hydroxyphenyl]-5-cyclopentyl-N-ethyl-1H-pyrazole-1- |
| | carboxamide |
| 598 | 3-[4-(acetylamino)-2-hydroxyphenyl]-N-allyl-5-cyclopentyl-1H-pyrazole-1- |
| | carboxamide |
| 599 | 3-[4-(acetylamino)-2-hydroxyphenyl]-5-cyclopentyl-N-propyl-1H-pyrazole-1- |
| | carboxamide |
| 600 | 3-[4-(acetylamino)-2-hydroxyphenyl]-N-benzyl-5-cyclopentyl-1H-pyrazole-1- |
| | carboxamide |
| 601 | 3-[4-(acetylamino)-2-hydroxyphenyl]-5-cyclopentyl-N-(2-phenylethyl)-1H- |
| | pyrazole-1-carboxamide |
| 602 | 3-[4-(acetylamino)-2-hydroxyphenyl]-5-cyclopentyl-N-(2-methylbenzyl)-1H- |
| | pyrazole-1-carboxamide |
| 603 | 3-[4-(acetylamino)-2-hydroxyphenyl]-5-cyclopentyl-N-(3-methylbenzyl)-1H- |
| | pyrazole-1-carboxamide |
| 604 | 3-[4-(acetylamino)-2-hydroxyphenyl]-5-cyclopentyl-N-(4-methylbenzyl)-1H- |
| | pyrazole-1-carboxamide |
| 605 | 3-[4-(acetylamino)-2-hydroxyphenyl]-N-(2-chlorobenzyl)-5-cyclopentyl-1H- |
| | pyrazole-1-carboxamide |
| 606 | 3-[4-(acetylamino)-2-hydroxyphenyl]-5-cyclopentyl-N-(4-fluorobenzyl)-1H- |
| | pyrazole-1-carboxamide |
| 607 | 3-[4-(acetylamino)-2-hydroxyphenyl]-5-cyclopentyl-N-(3,4-dichlorobenzyl)-1H- |
| | pyrazole-1-carboxamide |
| 608 | 3-[4-(acetylamino)-2-hydroxyphenyl]-5-cyclopentyl-N-(4-methoxybenzyl)-1H- |
| | pyrazole-1-carboxamide |
| 609 | 3-[4-(acetylamino)-2-hydroxyphenyl]-N-(sec-butyl)-5-cyclopentyl-1H-pyrazole-1- |
| | carboxamide |
| 610 | 3-[4-(acetylamino)-2-hydroxyphenyl]-5-cyclopentyl-N-(2-fluorobenzyl)-1H- |
| | pyrazole-1-carboxamide |
| 611 | 3-[4-(acetylamino)-2-hydroxyphenyl]-5-cyclopentyl-N-(3-fluorobenzyl)-1H- |
| | pyrazole-1-carboxamide |
| 612 | 3-[4-(acetylamino)-2-hydroxyphenyl]-5-cyclopentyl-N-cyclopropyl-1H-pyrazole- |
| | 1-carboxamide |
| 613 | 5-cyclopropyl-3-{2-hydroxy-4-[(3-methylbutanoyl)amino]phenyl}-N-isopropyl- |
| | 1H-pyrazole-1-carboxamide |
| 614 | 5-cyclopropyl-N-ethyl-3-{2-hydroxy-4-[(3-methylbutanoyl)amino]phenyl}-1H- |
| | pyrazole-1-carboxamide |
| 615 | N-allyl-5-cyclopropyl-3-{2-hydroxy-4-[(3-methylbutanoyl)amino]phenyl}-1H- |
| | pyrazole-1-carboxamide |
| 616 | 5-cyclopropyl-3-{2-hydroxy-4-[(3-methylbutanoyl)amino]phenyl}-N-propyl-1H- |
| | pyrazole-1-carboxamide |
| 617 | N-benzyl-5-cyclopropyl-3-{2-hydroxy-4-[(3-methylbutanoyl)amino]phenyl}-1H- |
| | pyrazole-1-carboxamide |
| 618 | 5-cyclopropyl-3-{2-hydroxy-4-[(3-methylbutanoyl)amino]phenyl}-N-(2- |
| | phenylethyl)-1H-pyrazole-1-carboxamide |
| 619 | 5-cyclopropyl-3-{2-hydroxy-4-[(3-methylbutanoyl)amino]phenyl}-N-(2- |
| | methylbenzyl)-1H-pyrazole-1-carboxamide |
| 620 | 5-cyclopropyl-3-{2-hydroxy-4-[(3-methylbutanoyl)amino]phenyl}-N-(3- |
| | methylbenzyl)-1H-pyrazole-1-carboxamide |
| 621 | 5-cyclopropyl-3-{2-hydroxy-4-[(3-methylbutanoyl)amino]phenyl}-N-(4- |
| | methylbenzyl)-1H-pyrazole-1-carboxamide |
| 622 | N-(2-chlorobenzyl)-5-cyclopropyl-3-{2-hydroxy-4-[(3- |
| | methylbutanoyl)amino]phenyl}-1H-pyrazole-1-carboxamide |
| 623 | 5-cyclopropyl-N-(4-fluorobenzyl)-3-{2-hydroxy-4-[(3- |
| | methylbutanoyl)amino]phenyl}-1H-pyrazole-1-carboxamide |
| 624 | 5-cyclopropyl-N-(3,4-dichlorobenzyl)-3-{2-hydroxy-4-[(3- |
| | methylbutanoyl)amino]phenyl}-1H-pyrazole-1-carboxamide |
| 625 | 5-cyclopropyl-3-{2-hydroxy-4-[(3-methylbutanoyl)amino]phenyl}-N-(4- |
| | methoxybenzyl)-1H-pyrazole-1-carboxamide |
| 626 | N-(sec-butyl)-5-cyclopropyl-3-{2-hydroxy-4-[(3-methylbutanoyl)amino]phenyl}- |
| | 1H-pyrazole-1-carboxamide |
| 627 | 5-cyclopropyl-N-(2-fluorobenzyl)-3-{2-hydroxy-4-[(3- |
| | methylbutanoyl)amino]phenyl}-1H-pyrazole-1-carboxamide |
| 628 | 5-cyclopropyl-N-(3-fluorobenzyl)-3-{2-hydroxy-4-[(3- |
| | methylbutanoyl)amino]phenyl}-1H-pyrazole-1-carboxamide |
| 629 | N,5-dicyclopropyl-3-{2-hydroxy-4-[(3-methylbutanoyl)amino]phenyl}-1H- |
| | pyrazole-1-carboxamide |
| 630 | 5-cyclobutyl-N-ethyl-3-{2-hydroxy-4-[(3-methylbutanoyl)amino]phenyl}-1H- |
| | pyrazole-1-carboxamide |
| 631 | 5-cyclobutyl-N-ethyl-3-{2-hydroxy-4-[(3-methylbutanoyl)amino]phenyl}-1H- |
| | pyrazole-1-carboxamide |
| 632 | N-allyl-5-cyclobutyl-3-{2-hydroxy-4-[(3-methylbutanoyl)amino]phenyl}-1H- |
| | pyrazole-1-carboxamide |
| 633 | 5-cyclobutyl-3-{2-hydroxy-4-[(3 -methylbutanoyl)amino]phenyl}-N-propyl-1H- |
| | pyrazole-1-carboxamide |
| 634 | N-benzyl-5-cyclobutyl-3-{2-hydroxy-4-[(3-methylbutanoyl)amino]phenyl}-1H- |
| | pyrazole-1-carboxamide |
| 635 | 5-cyclobutyl-3-{2-hydroxy-4-[(3-methylbutanoyl)amino]phenyl}-N-(2- |
| | phenylethyl)-1H-pyrazole-1-carboxamide |
| 636 | 5-cyclobutyl-3-{2-hydroxy-4-[(3-methylbutanoyl)amino]phenyl}-N-(2- |
| | methylbenzyl)-1H-pyrazole-1-carboxamide |
| 637 | 5-cyclobutyl-3-{2-hydroxy-4-[(3-methylbutanoyl)amino]phenyl}-N-(3- |
| | methylbenzyl)-1H-pyrazole-1-carboxamide |
| 638 | 5-cyclobutyl-3-{2-hydroxy-4-[(3-methylbutanoyl)amino]phenyl}-N-(4- |
| | methylbenzyl)-1H-pyrazole-1-carboxamide |
| 639 | N-(2-chlorobenzyl)-5-cyclobutyl-3-{2-hydroxy-4-[(3- |
| | methylbutanoyl)amino]phenyl}-1H-pyrazole-1-carboxamide |
| 640 | 5-cyclobutyl-N-(4-fluorobenzyl)-3-{2-hydroxy-4-[(3- |
| | methylbutanoyl)amino]phenyl}-1H-pyrazole-1-carboxamide |
| 641 | 5-cyclobutyl-N-(3,4-dichlorobenzyl)-3-{2-hydroxy-4-[(3- |
| | methylbutanoyl)amino]phenyl}-1H-pyrazole-1-carboxamide |
| 642 | 5-cyclobutyl-3-{2-hydroxy-4-[(3-methylbutanoyl)amino]phenyl}-N-(4- |
| | methoxybenzyl)-1H-pyrazole-1-carboxamide |
| 643 | N-(sec-butyl)-5-cyclobutyl-3-{2-hydroxy-4-[(3-methylbutanoyl)amino]phenyl}- |
| | 1H-pyrazole-1-carboxamide |
| 644 | 5-cyclobutyl-N-(2-fluorobenzyl)-3-{2-hydroxy-4-[(3- |
| | methylbutanoyl)amino]phenyl}-1H-pyrazole-1-carboxamide |
| 645 | 5-cyclobutyl-N-(3-fluorobenzyl)-3-{2-hydroxy-4-[(3- |
| | methylbutanoyl)amino]phenyl}-1H-pyrazole-1-carboxamide |
| 646 | 5-cyclobutyl-N-cyclopropyl-3-{2-hydroxy-4-[(3-methylbutanoyl)amino]phenyl}- |
| | 1H-pyrazole-1-carboxamide |
| 647 | 5-cyclopentyl-3-{2-hydroxy-4-[(3-methylbutanoyl)amino]phenyl}-N-isopropyl- |
| | 1H-pyrazole-1-carboxamide |
| 648 | 5-cyclopentyl-N-ethyl-3-{2-hydroxy-4-[(3-methylbutanoyl)amino]phenyl}-1H- |
| | pyrazole-1-carboxamide |
| 649 | N-allyl-5-cyclopentyl-3-{2-hydroxy-4-[(3-methylbutanoyl)amino]phenyl}-1H- |
| | pyrazole-1-carboxamide |
| 650 | 5-cyclopentyl-3-{2-hydroxy-4-[(3-methylbutanoyl)amino]phenyl}-N-propyl-1H- |
| | pyrazole-1-carboxamide |
| 651 | N-benzyl-5-cyclopentyl-3-{2-hydroxy-4-[(3-methylbutanoyl)amino]phenyl}-1H- |
| | pyrazole-1-carboxamide |
| 652 | 5-cyclopentyl-3-{2-hydroxy-4-[(3-methylbutanoyl)amino]phenyl}-N-(2- |
| | phenylethyl)-1H-pyrazole-1-carboxamide |
| 653 | 5-cyclopentyl-3-{2-hydroxy-4-[(3-methylbutanoyl)amino]phenyl}-N-(2- |
| | methylbenzyl)-1H-pyrazole-1-carboxamide |
| 654 | 5-cyclopentyl-3-{2-hydroxy-4-[(3-methylbutanoyl)amino]phenyl}-N-(3- |
| | methylbenzyl)-1H-pyrazole-1-carboxamide |
| 655 | 5-cyclopentyl-3-{2-hydroxy-4-[(3-methylbutanoyl)amino]phenyl}-N-(4- |
| | methylbenzyl)-1H-pyrazole-1-carboxamide |
| 656 | N-(2-chlorobenzyl)-5-cyclopentyl-3-{2-hydroxy-4-[(3- |
| | methylbutanoyl)amino]phenyl}-1H-pyrazole-1-carboxamide |
| 657 | 5-cyclopentyl-N-(4-fluorobenzyl)-3-{2-hydroxy-4-[(3- |
| | methylbutanoyl)amino]phenyl}-1H-pyrazole-1-carboxamide |
| 658 | 5-cyclopentyl-N-(3,4-dichlorobenzyl)-3-{2-hydroxy-4-[(3- |
| | methylbutanoyl)amino]phenyl}-1H-pyrazole-1-carboxamide |
| 659 | 5-cyclopentyl-3-{2-hydroxy-4-[(3-methylbutanoyl)amino]phenyl}-N-(4- |
| | methoxybenzyl)-1H-pyrazole-1-carboxamide |
| 660 | N-(sec-butyl)-5-cyclopentyl-3-{2-hydroxy-4-[(3-methylbutanoyl)amino]phenyl}- |
| | 1H-pyrazole-1-carboxamide |
| 661 | 5-cyclopentyl-N-(2-fluorobenzyl)-3-{2-hydroxy-4-[(3- |
| | methylbutanoyl)amino]phenyl}-1H-pyrazole-1-carboxamide |
| 662 | 5-cyclopentyl-N-(3-fluorobenzyl)-3-{2-hydroxy-4-[(3- |
| | methylbutanoyl)amino]phenyl}-1H-pyrazole-1-carboxamide |
| 663 | 5-cyclopentyl-N-cyclopropyl-3-{2-hydroxy-4-[(3-methylbutanoyl)amino]phenyl}- |
| | 1H-pyrazole-1-carboxamide |
| 664 | 5-cyclopropyl-3-[2-hydroxy-4-(propionylamino)phenyl]-N-isopropyl-1H- |
| | pyrazole-1-carboxamide |
| 665 | 5-cyclopropyl-N-ethyl-3-[2-hydroxy-4-(propionylamino)phenyl]-1H-pyrazole-1- |
| | carboxamide |
| 666 | N-allyl-5-cyclopropyl-3-[2-hydroxy-4-(propionylamino)phenyl]-1H-pyrazole-1- |
| | carboxamide |
| 667 | 5-cyclopropyl-3-[2-hydroxy-4-(propionylamino)phenyl]-N-propyl-1H-pyrazole-1- |
| | carboxamide |
| 668 | N-benzyl-5-cyclopropyl-3-[2-hydroxy-4-(propionylamino)phenyl]-1H-pyrazole-1- |
| | carboxamide |
| 669 | 5-cyclopropyl-3-[2-hydroxy-4-(propionylamino)phenyl]-N-(2-phenylethyl)-1H- |
| | pyrazole-1-carboxamide |
| 670 | 5-cyclopropyl-3-[2-hydroxy-4-(propionylamino)phenyl]-N-(2-methylbenzyl)-1H- |
| | pyrazole-1-carboxamide |
| 671 | 5-cyclopropyl-3-[2-hydroxy-4-(propionylamino)phenyl]-N-(3-methylbenzyl)-1H- |
| | pyrazole-1-carboxamide |
| 672 | N,5-dicyclopropyl-3-[2-hydroxy-4-(propionylamino)phenyl]-1H-pyrazole-1- |
| | carboxamide |
| 673 | N-(2-chlorobenzyl)-5-cyclopropyl-3-[2-hydroxy-4-(propionylamino)phenyl]-1H- |
| | pyrazole-1-carboxamide |
| 674 | 5-cyclopropyl-N-(4-fluorobenzyl)-3-[2-hydroxy-4-(propionylamino)phenyl]-1H- |
| | pyrazole-1-carboxamide |
| 675 | 5-cyclopropyl-N-(3,4-dichlorobenzyl)-3-[2-hydroxy-4-(propionylamino)phenyl]- |
| | 1H-pyrazole-1-carboxamide |
| 676 | 5-cyclopropyl-3-[2-hydroxy-4-(propionylamino)phenyl]-N-(4-methoxybenzyl)- |
| | 1H-pyrazole-1-carboxamide |
| 677 | N-(sec-butyl)-5-cyclopropyl-3-[2-hydroxy-4-(propionylamino)phenyl]-1H- |
| | pyrazole-1-carboxamide |
| 678 | 5-cyclopropyl-N-(2-fluorobenzyl)-3-[2-hydroxy-4-(propionylamino)phenyl]-1H- |
| | pyrazole-1-carboxamide |
| 679 | 5-cyclopropyl-N-(3-fluorobenzyl)-3-[2-hydroxy-4-(propionylamino)phenyl]-1H- |
| | pyrazole-1-carboxamide |
| 680 | N,5-dicyclopropyl-3-[2-hydroxy-4-(propionylamino)phenyl]-1H-pyrazole-1- |
| | carboxamide |
| 681 | 5-cyclobutyl-3-[2-hydroxy-4-(propionylamino)phenyl]-N-isopropyl-1H-pyrazole- |
| | 1-carboxamide |
| 682 | 5-cyclobutyl-N-ethyl-3-[2-hydroxy-4-(propionylamino)phenyl]-1H-pyrazole-1- |
| | carboxamide |
| 683 | N-allyl-5-cyclobutyl-3-[2-hydroxy-4-(propionylamino)phenyl]-1H-pyrazole-1- |
| | carboxamide |
| 684 | 5-cyclobutyl-3-[2-hydroxy-4-propionylamino)phenyl]-N-propyl-1H-pyrazole-1- |
| | carboxamide |
| 685 | N-benzyl-5-cyclobutyl-3-[2-hydroxy-4-(propionylamino)phenyl]-1H-pyrazole-1- |
| | carboxamide |
| 686 | 5-cyclobutyl-3-[2-hydroxy-4-(propionylamino)phenyl]-N-(2-phenylethyl)-1H- |
| | pyrazole-1-carboxamide |
| 687 | 5-cyclobutyl-3-[2-hydroxy-4-(propionylamino)phenyl]-N-(2-methylbenzyl)-1H- |
| | pyrazole-1-carboxamide |
| 688 | 5-cyclobutyl-3-[2-hydroxy-4-(propionylamino)phenyl]-N-(3-methylbenzyl)-1H- |
| | pyrazole-1-carboxamide |
| 689 | 5-cyclobutyl-3-[2-hydroxy-4-(propionylamino)phenyl]-N-(4-methylbenzyl)-1H- |
| | pyrazole-1-carboxamide |
| 690 | N-(2-chlorobenzyl)-5-cyclobutyl-3-[2-hydroxy-4-(propionylamino)phenyl]-1H- |
| | pyrazole-1-carboxamide |
| 691 | 5-cyclobutyl-N-(4-fluorobenzyl)-3-[2-hydroxy-4-(propionylamino)phenyl]-1H- |
| | pyrazole-1-carboxamide |
| 692 | 5-cyclobutyl-N-(3,4-dichlorobenzyl)-3-[2-hydroxy-4-(propionylamino)phenyl]- |
| | 1H-pyrazole-1-carboxamide |
| 693 | 5-cyclobutyl-3-[2-hydroxy-4-(propionylamino)phenyl]-N-(4-methoxybenzyl)-1H- |
| | pyrazole-1-carboxamide |
| 694 | N-(sec-butyl)-5-cyclobutyl-3-[2-hydroxy-4-(propionylamino)phenyl]-1H- |
| | pyrazole-1-carboxamide |
| 695 | 5-cyclobutyl-N-(2-fluorobenzyl)-3-[2-hydroxy-4-(propionylamino)phenyl]-1H- |
| | pyrazole-1-carboxamide |
| 696 | 5-cyclobutyl-N-(3-fluorobenzyl)-3-[2-hydroxy-4-(propionylamino)phenyl]-1H- |
| | pyrazole-1-carboxamide |
| 697 | 5-cyclobutyl-N-cyclopropyl-3-[2-hydroxy-4-(propionylamino)phenyl]-1H- |
| | pyrazole-1-carboxamide |
| 698 | 5-cyclopentyl-3-[2-hydroxy-4-(propionylamino)phenyl]-N-isopropyl-1H-pyrazole- |
| | 1-carboxamide |
| 699 | 5-cyclopentyl-N-ethyl-3-[2-hydroxy-4-(propionylamino)phenyl]-1H-pyrazole-1- |
| | carboxamide |
| 700 | N-allyl-5-cyclopentyl-3-[2-hydroxy-4-(propionylamino)phenyl]-1H-pyrazole-1- |
| | carboxamide |
| 701 | 5-cyclopentyl-3-[2-hydroxy-4-(propionylamino)phenyl]-N-propyl-1H-pyrazole-1- |
| | carboxamide |
| 702 | N-benzyl-5-cyclopentyl-3-[2-hydroxy-4-(propionylamino)phenyl]-1H-pyrazole-1- |
| | carboxamide |
| 703 | 5-cyclopentyl-3-[2-hydroxy-4-(propionylamino)phenyl]-N-(2-phenylethyl)-1H- |
| | pyrazole-1-carboxamide |
| 704 | 5-cyclopentyl-3-[2-hydroxy-4-(propionylamino)phenyl]-N-(2-methylbenzyl)-1H- |
| | pyrazole-1-carboxamide |
| 705 | 5-cyclopentyl-3-[2-hydroxy-4-(propionylamino)phenyl]-N-(3-methylbenzyl)-1H- |
| | pyrazole-1-carboxamide |
| 706 | 5-cyclopentyl-3-[2-hydroxy-4-(propionylamino)phenyl]-N-(4-methylbenzyl)-1H- |
| | pyrazole-1-carboxamide |
| 707 | N-(2-chlorobenzyl)-5-cyclopentyl-3-[2-hydroxy-4-(propionylamino)phenyl]-1H- |
| | pyrazole-1-carboxamide |
| 708 | 5-cyclopentyl-N-(4-fluorobenzyl)-3-[2-hydroxy-4-(propionylamino)phenyl]-1H- |
| | pyrazole-1-carboxamide |
| 709 | 5-cyclopentyl-N-(3,4-dichlorobenzyl)-3-[2-hydroxy-4-(propionylamino)phenyl]- |
| | 1H-pyrazole-1-carboxamide |
| 710 | 5-cyclopentyl-3-[2-hydroxy-4-(propionylamino)phenyl]-N-(4-methoxybenzyl)- |
| | 1H-pyrazole-1-carboxamide |
| 711 | N-(sec-butyl)-5-cyclopentyl-3-[2-hydroxy-4-(propionylamino)phenyl]-1H- |
| | pyrazole-1-carboxamide |
| 712 | 5-cyclopentyl-N-(2-fluorobenzyl)-3-[2-hydroxy-4-(propionylamino)phenyl]-1H- |
| | pyrazole-1-carboxamide |
| 713 | 5-cyclopentyl-N-(3-fluorobenzyl)-3-[2-hydroxy-4-(propionylamino)phenyl]-1H- |
| | pyrazole-1-carboxamide |
| 714 | 5-cyclopentyl-N-cyclopropyl-3-[2-hydroxy-4-(propionylamino)phenyl]-1H- |
| | pyrazole-1-carboxamide |
| 715 | 5-cyclopropyl-3-{4-[(cyclopropylcarbonyl)amino]-2-hydroxyphenyl}-N- |
| | isopropyl-1H-pyrazole-1-carboxamide |
| 716 | 5-cyclopropyl-3-{4-[(cyclopropylcarbonyl)amino]-2-hydroxyphenyl}-N-ethyl-1H- |
| | pyrazole-1-carboxamide |
| 717 | N-allyl-5-cyclopropyl-3-{4-[(cyclopropylcarbonyl)amino]-2-hydroxyphenyl}-1H- |
| | pyrazole-1-carboxamide |
| 718 | 5-cyclopropyl-3-{4-[(cyclopropylcarbonyl)amino]-2-hydroxyphenyl}-N-propyl- |
| | 1H-pyrazole-1-carboxamide |
| 719 | N-benzyl-5-cyclopropyl-3-{4-[(cyclopropylcarbonyl)amino]-2-hydroxyphenyl}- |
| | 1H-pyrazole-1-carboxamide |
| 720 | 5-cyclopropyl-3-{4-[(cyclopropylcarbonyl)amino]-2-hydroxyphenyl}-N-(2- |
| | phenylethyl)-1H-pyrazole-1-carboxamide |
| 721 | 5-cyclopropyl-3-{4-[(cyclopropylcarbonyl)amino]-2-hydroxyphenyl}-N-(2- |
| | methylbenzyl)-1H-pyrazole-1-carboxamide |
| 722 | 5-cyclopropyl-3-{4-[(cyclopropylcarbonyl)amino]-2-hydroxyphenyl}-N-(3- |
| | methylbenzyl)-1H-pyrazole-1-carboxamide |
| 723 | 5-cyclopropyl-3-{4-[(cyclopropylcarbonyl)amino]-2-hydroxyphenyl}-N-(4- |
| | methylbenzyl)-1H-pyrazole-1-carboxamide |
| 724 | N-(2-chlorobenzyl)-5-cyclopropyl-3-{4-[(cyclopropylcarbonyl)amino]-2- |
| | hydroxyphenyl}-1H-pyrazole-1-carboxamide |
| 725 | 5-cyclopropyl-3-{4-[(cyclopropylcarbonyl)amino]-2-hydroxyphenyl}-N-(4- |
| | fluorobenzyl)-1H-pyrazole-1-carboxamide |
| 726 | 5-cyclopropyl-3-{4-[(cyclopropylcarbonyl)amino]-2-hydroxyphenyl}-N-(3,4- |
| | dichlorobenzyl)-1H-pyrazole-1-carboxamide |
| 727 | 5-cyclopropyl-3-{4-[(cyclopropylcarbonyl)amino]-2-hydroxyphenyl}-N-(4- |
| | methoxybenzyl)-1H-pyrazole-1-carboxamide |
| 728 | N-(sec-butyl)-5-cyclopropyl-3-{4-[(cyclopropylcarbonyl)amino]-2- |
| | hydroxyphenyl}-1H-pyrazole-1-carboxamide |
| 729 | 5-cyclopropyl-3-{4-[(cyclopropylcarbonyl)amino]-2-hydroxyphenyl}-N-(2- |
| | fluorobenzyl)-1H-pyrazole-1-carboxamide |
| 730 | 5-cyclopropyl-3-{4-[(cyclopropylcarbonyl)amino]-2-hydroxyphenyl}-N-(3- |
| | fluorobenzyl)-1H-pyrazole-1-carboxamide |
| 731 | N,5-dicyclopropyl-3-{4-[(cyclopropylcarbonyl)amino]-2-hydroxyphenyl}-1H- |
| | pyrazole-1-carboxamide |
| 732 | 5-cyclobutyl-3-{4-[(cyclopropylcarbonyl)amino]-2-hydroxyphenyl}-N-isopropyl- |
| | 1H-pyrazole-1-carboxamide |
| 733 | 5-cyclobutyl-3-{4-[(cyclopropylcarbonyl)amino]-2-hydroxyphenyl}-N-ethyl-1H- |
| | pyrazole-1-carboxamide |
| 734 | N-allyl-5-cyclobutyl-3-{4-[(cyclopropylcarbonyl)amino]-2-hydroxyphenyl}-1H- |
| | pyrazole-1-carboxamide |
| 735 | 5-cyclobutyl-3-{4-[(cyclopropylcarbonyl)amino]-2-hydroxyphenyl}-N-propyl-1H- |
| | pyrazole-1-carboxamide |
| 736 | N-benzyl-5-cyclobutyl-3-{4-[(cyclopropylcarbonyl)amino]-2-hydroxyphenyl}-1H- |
| | pyrazole-1-carboxamide |
| 737 | 5-cyclobutyl-3-{4-[(cyclopropylcarbonyl)amino]-2-hydroxyphenyl}-N-(2- |
| | phenylethyl)-1H-pyrazole-1-carboxamide |
| 738 | 5-cyclobutyl-3-{4-[(cyclopropylcarbonyl)amino]-2-hydroxyphenyl}-N-(2- |
| | methylbenzyl)-1H-pyrazole-1-carboxamide |
| 739 | 5-cyclobutyl-3-{4-[(cyclopropylcarbonyl)amino]-2-hydroxyphenyl}-N-(3- |
| | methylbenzyl)-1H-pyrazole-1-carboxamide |
| 740 | 5-cyclobutyl-3-{4-[(cyclopropylcarbonyl)amino]-2-hydroxyphenyl}-N-(4- |
| | methylbenzyl)-1H-pyrazole-1-carboxamide |
| 741 | N-(2-chlorobenzyl)-5-cyclobutyl-3-{4-[(cyclopropylcarbonyl)amino]-2- |
| | hydroxyphenyl}-1H-pyrazole-1-carboxamide |
| 742 | 5-cyclobutyl-3-{4-[(cyclopropylcarbonyl)amino]-2-hydroxyphenyl}-N-(4- |
| | fluorobenzyl)-1H-pyrazole-1-carboxamide |
| 743 | 5-cyclobutyl-3-{4-[(cyclopropylcarbonyl)amino]-2-hydroxyphenyl}-N-(3,4- |
| | dichlorobenzyl)-1H-pyrazole-1-carboxamide |
| 744 | 5-cyclobutyl-3-{4-[(cyclopropylcarbonyl)amino]-2-hydroxyphenyl}-N-(4- |
| | methoxybenzyl)-1H-pyrazole-1-carboxamide |
| 745 | N-(sec-butyl)-5-cyclobutyl-3-{4-[(cyclopropylcarbonyl)amino]-2- |
| | hydroxyphenyl}-1H-pyrazole-1-carboxamide |
| 746 | 5-cyclobutyl-3-{4-[(cyclopropylcarbonyl)amino]-2-hydroxyphenyl}-N-(2- |
| | fluorobenzyl)-1H-pyrazole-1-carboxamide |
| 747 | 5-cyclobutyl-3-{4-[(cyclopropylcarbonyl)amino]-2-hydroxyphenyl}-N-(3- |
| | fluorobenzyl)-1H-pyrazole-1-carboxamide |
| 748 | 5-cyclobutyl-N-cyclopropyl-3-{4-[(cyclopropylcarbonyl)amino]-2- |
| | hydroxyphenyl}-1H-pyrazole-1-carboxamide |
| 749 | 5-cyclopentyl-3-{4-[(cyclopropylcarbonyl)amino]-2-hydroxyphenyl}-N-isopropyl- |
| | 1H-pyrazole-1-carboxamide |
| 750 | 5-cyclopentyl-3-{4-[(cyclopropylcarbonyl)amino]-2-hydroxyphenyl}-N-ethyl-1H- |
| | pyrazole-1-carboxamide |
| 751 | N-allyl-5-cyclopentyl-3-{4-[(cyclopropylcarbonyl)amino]-2-hydroxyphenyl}-1H- |
| | pyrazole-1-carboxamide |
| 752 | 5-cyclopentyl-3-{4-[(cyclopropylcarbonyl)amino]-2-hydroxyphenyl}-N-propyl- |
| | 1H-pyrazole-1-carboxamide |
| 753 | N-benzyl-5-cyclopentyl-3-{4-[(cyclopropylcarbonyl)amino]-2-hydroxyphenyl}- |
| | 1H-pyrazole-1-carboxamide |
| 754 | 5-cyclopentyl-3-{4-[(cyclopropylcarbonyl)amino]-2-hydroxyphenyl}-N-(2- |
| | phenylethyl)-1H-pyrazole-1-carboxamide |
| 755 | 5-cyclopentyl-3-{4-[(cyclopropylcarbonyl)amino]-2-hydroxyphenyl}-N-(2- |
| | methylbenzyl)-1H-pyrazole-1-carboxamide |
| 756 | 5-cyclopentyl-3-{4-[(cyclopropylcarbonyl)amino]-2-hydroxyphenyl}-N-(3- |
| | methylbenzyl)-1H-pyrazole-1-carboxamide |
| 757 | 5-cyclopentyl-3-{4-[(cyclopropylcarbonyl)amino]-2-hydroxyphenyl}-N-(4- |
| | methylbenzyl)-1H-pyrazole-1-carboxamide |
| 758 | N-(2-chlorobenzyl)-5-cyclopentyl-3-{4-[(cyclopropylcarbonyl)amino]-2- |
| | hydroxyphenyl}-1H-pyrazole-1-carboxamide |
| 759 | 5-cyclopentyl-3-{4-[(cyclopropylcarbonyl)amino]-2-hydroxyphenyl}-N-(4- |
| | fluorobenzyl)-1H-pyrazole-1-carboxamide |
| 760 | 5-cyclopentyl-3-{4-[(cyclopropylcarbonyl)amino]-2-hydroxyphenyl}-N-(3,4- |
| | dichlorobenzyl)-1H-pyrazole-1-carboxamide |
| 761 | 5-cyclopentyl-3-{4-[(cyclopropylcarbonyl)amino]-2-hydroxyphenyl}-N-(4- |
| | methoxybenzyl)-1H-pyrazole-1-carboxamide |
| 762 | N-(sec-butyl)-5-cyclopentyl-3-{4-[(cyclopropylcarbonyl)amino]-2- |
| | hydroxyphenyl}-1H-pyrazole-1-carboxamide |
| 763 | 5-cyclopentyl-3-{4-[(cyclopropylcarbonyl)amino]-2-hydroxyphenyl}-N-(2- |
| | fluorobenzyl)-1H-pyrazole-1-carboxamide |
| 764 | 5-cyclopentyl-3-{4-[(cyclopropylcarbonyl)amino]-2-hydroxyphenyl}-N-(3- |
| | fluorobenzyl)-1H-pyrazole-1-carboxamide |
| 765 | 5-cyclopentyl-N-cyclopropyl-3-{4-[(cyclopropylcarbonyl)amino]-2- |
| | hydroxyphenyl}-1H-pyrazole-1-carboxamide |
| 766 | 5-cyclopropyl-3-[4-(2-furoylamino)-2-hydroxyphenyl]-N-isopropyl-1H-pyrazole- |
| | 1-carboxamide |
| 767 | 5-cyclopropyl-N-ethyl-3-[4-(2-furoylamino)-2-hydroxyphenyl]-1H-pyrazole-1- |
| | carboxamide |
| 768 | N-allyl-5-cyclopropyl-3-[4-(2-furoylamino)-2-hydroxyphenyl]-1H-pyrazole-1- |
| | carboxamide |
| 769 | 5-cyclopropyl-3-[4-(2-furoylamino)-2-hydroxyphenyl]-N-propyl-1H-pyrazole-1- |
| | carboxamide |
| 770 | N-benzyl-5-cyclopropyl-3-[4-(2-furoylamino)-2-hydroxyphenyl]-1H-pyrazole-1- |
| | carboxamide |
| 771 | 5-cyclopropyl-3-[4-(2-furoylamino)-2-hydroxyphenyl]-N-(2-phenylethyl)-1H- |
| | pyrazole-1-carboxamide |
| 772 | 5-cyclopropyl-3-[4-(2-furoylamino)-2-hydroxyphenyl]-N-(2-methylbenzyl)-1H- |
| | pyrazole-1-carboxamide |
| 773 | 5-cyclopropyl-3-[4-(2-furoylamino)-2-hydroxyphenyl]-N-(3-methylbenzyl)-1H- |
| | pyrazole-1-carboxamide |
| 774 | 5-cyclopropyl-3-[4-(2-furoylamino)-2-hydroxyphenyl]-N-(4-methylbenzyl)-1H- |
| | pyrazole-1-carboxamide |
| 775 | N-(2-chlorobenzyl)-5-cyclopropyl-3-[4-(2-furoylamino)-2-hydroxyphenyl]-1H- |
| | pyrazole-1-carboxamide |
| 776 | 5-cyclopropyl-N-(4-fluorobenzyl)-3-[4-(2-furoylamino)-2-hydroxyphenyl]-1H- |
| | pyrazole-1-carboxamide |
| 777 | 5-cyclopropyl-N-(3,4-dichlorobenzyl)-3-[4-(2-furoylamino)-2-hydroxyphenyl]- |
| | 1H-pyrazole-1-carboxamide |
| 778 | 5-cyclopropyl-3-[4-(2-furoylamino)-2-hydroxyphenyl]-N-(4-methoxybenzyl)-1H- |
| | pyrazole-1-carboxamide |
| 779 | N-(sec-butyl)-5-cyclopropyl-3-[4-(2-furoylamino)-2-hydroxyphenyl]-1H-pyrazole- |
| | 1-carboxamide |
| 780 | 5-cyclopropyl-N-(2-fluorobenzyl)-3-[4-(2-furoylamino)-2-hydroxyphenyl]-1H- |
| | pyrazole-1-carboxamide |
| 781 | 5-cyclopropyl-N-(3-fluorobenzyl)-3-[4-(2-furoylamino)-2-hydroxyphenyl]-1H- |
| | pyrazole-1-carboxamide |
| 782 | N,5-dicyclopropyl-3-[4-(2-furoylamino)-2-hydroxyphenyl]-1H-pyrazole-1- |
| | carboxamide |
| 783 | 5-cyclobutyl-3-[4-(2-furoylamino)-2-hydroxyphenyl]-N-isopropyl-1H-pyrazole-1- |
| | carboxamide |
| 784 | 5-cyclobutyl-N-ethyl-3-[4-(2-furoylamino)-2-hydroxyphenyl]-1H-pyrazole-1- |
| | carboxamide |
| 785 | N-allyl-5-cyclobutyl-3-[4-(2-furoylamino)-2-hydroxyphenyl]-1H-pyrazole-1- |
| | carboxamide |
| 786 | 5-cyclobutyl-3-[4-(2-furoylamino)-2-hydroxyphenyl]-N-propyl-1H-pyrazole-1- |
| | carboxamide |
| 787 | N-benzyl-5-cyclobutyl-3-[4-(2-furoylamino)-2-hydroxyphenyl]-1H-pyrazole-1- |
| | carboxamide |
| 788 | 5-cyclobutyl-3-[4-(2-furoylamino)-2-hydroxyphenyl]-N-(2-phenylethyl)-1H- |
| | pyrazole-1-carboxamide |
| 789 | 5-cyclobutyl-3-[4-(2-furoylamino)-2-hydroxyphenyl]-N-(2-methylbenzyl)-1H- |
| | pyrazole-1-carboxamide |
| 790 | 5-cyclobutyl-3-[4-(2-furoylamino)-2-hydroxyphenyl]-N-(3-methylbenzyl)-1H- |
| | pyrazole-1-carboxamide |
| 791 | 5-cyclobutyl-3-[4-(2-furoylamino)-2-hydroxyphenyl]-N-(4-methylbenzyl)-1H- |
| | pyrazole-1-carboxamide |
| 792 | N-(2-chlorobenzyl)-5-cyclobutyl-3-[4-(2-furoylamino)-2-hydroxyphenyl]-1H- |
| | pyrazole-1-carboxamide |
| 793 | 5-cyclobutyl-N-(4-fluorobenzyl)-3-[4-(2-furoylamino)-2-hydroxyphenyl]-1H- |
| | pyrazole-1-carboxamide |
| 794 | 5-cyclobutyl-N-(3,4-dichlorobenzyl)-3-[4-(2-furoylamino)-2-hydroxyphenyl]-1H- |
| | pyrazole-1-carboxamide |
| 795 | 5-cyclobutyl-3-[4-(2-furoylamino)-2-hydroxyphenyl]-N-(4-methoxybenzyl)-1H- |
| | pyrazole-1-carboxamide |
| 796 | N-(sec-butyl)-5-cyclobutyl-3-[4-(2-furoylamino)-2-hydroxyphenyl]-1H-pyrazole- |
| | 1-carboxamide |
| 797 | 5-cyclobutyl-N-(2-fluorobenzyl)-3-[4-(2-furoylamino)-2-hydroxyphenyl]-1H- |
| | pyrazole-1-carboxamide |
| 798 | 5-cyclobutyl-N-(3-fluorobenzyl)-3-[4-(2-furoylamino)-2-hydroxyphenyl]-1H- |
| | pyrazole-1-carboxamide |
| 799 | 5-cyclobutyl-N-cyclopropyl-3-[4-(2-furoylamino)-2-hydroxyphenyl]-1H-pyrazole- |
| | 1-carboxamide |
| 800 | 5-cyclopentyl-3-[4-(2-furoylamino)-2-hydroxyphenyl]-N-isopropyl-1H-pyrazole- |
| | 1-carboxamide |
| 801 | 5-cyclopentyl-N-ethyl-3-[4-(2-furoylamino)-2-hydroxyphenyl]-1H-pyrazole-1- |
| | carboxamide |
| 802 | N-allyl-5-cyclopentyl-3-[4-(2-furoylamino)-2-hydroxyphenyl]-1H-pyrazole-1- |
| | carboxamide |
| 803 | 5-cyclopentyl-3-[4-(2-furoylamino)-2-hydroxyphenyl]-N-propyl-1H-pyrazole-1- |
| | carboxamide |
| 804 | 5-cyclopentyl-3-[4-(2-furoylamino)-2-hydroxyphenyl]-N-(2-methylbenzyl)-1H- |
| | pyrazole-1-carboxamide |
| 805 | 5-cyclopentyl-3-[4-(2-furoylamino)-2-hydroxyphenyl]-N-(2-phenylethyl)-1H- |
| | pyrazole-1-carboxamide |
| 806 | 5-cyclopentyl-3-[4-(2-furoylamino)-2-hydroxyphenyl]-N-(2-methylbenzyl)-1H- |
| | pyrazole-1-carboxamide |
| 807 | 5-cyclopentyl-3-[4-(2-furoylamino)-2-hydroxyphenyl]-N-(3-methylbenzyl)-1H- |
| | pyrazole-1-carboxamide |
| 808 | 5-cyclopentyl-3-[4-(2-furoylamino)-2-hydroxyphenyl]-N-(4-methylbenzyl)-1H- |
| | pyrazole-1-carboxamide |
| 809 | N-(2-chlorobenzyl)-5-cyclopentyl-3-[4-(2-furoylamino)-2-hydroxyphenyl]-1H- |
| | pyrazole-1-carboxamide |
| 810 | 5-cyclopentyl-N-(4-fluorobenzyl)-3-[4-(2-furoylamino)-2-hydroxyphenyl]-1H- |
| | pyrazole-1-carboxamide |
| 811 | 5-cyclopentyl-N-(3,4-dichlorobenzyl)-3-[4-(2-furoylamino)-2-hydroxyphenyl]- |
| | 1H-pyrazole-1-carboxamide |
| 812 | 5-cyclopentyl-3-[4-(2-furoylamino)-2-hydroxyphenyl]-N-(4-methoxybenzyl)-1H- |
| | pyrazole-1-carboxamide |
| 813 | N-(sec-butyl)-5-cyclopentyl-3-[4-(2-furoylamino)-2-hydroxyphenyl]-1H-pyrazole- |
| | 1-carboxamide |
| 814 | 5-cyclopentyl-N-(2-fluorobenzyl)-3-[4-(2-furoylamino)-2-hydroxyphenyl]-1H- |
| | pyrazole-1-carboxamide |
| 815 | 5-cyclopentyl-N-(3-fluorobenzyl)-3-[4-(2-furoylamino)-2-hydroxyphenyl]-1H- |
| | pyrazole-1-carboxamide |
| 816 | 5-cyclopentyl-N-cyclopropyl-3-[4-(2-furoylamino)-2-hydroxyphenyl]-1H- |
| | pyrazole-1-carboxamide |
| 817 | 5-cyclopropyl-3-{2-hydroxy-4-[(thien-2-ylcarbonyl)amino]phenyl}-N-isopropyl- |
| | 1H-pyrazole-1-carboxamide |
| 818 | 5-cyclopropyl-N-ethyl-3-{2-hydroxy-4-[(thien-2-ylcarbonyl)amino]phenyl}-1H- |
| | pyrazole-1-carboxamide |
| 819 | N-allyl-5-cyclopropyl-3-{2-hydroxy-4-[(thien-2-ylcarbonyl)amino]phenyl}-1H- |
| | pyrazole-1-carboxamide |
| 820 | 5-cyclopropyl-3-{2-hydroxy-4-[(thien-2-ylcarbonyl)amino]phenyl}-N-propyl-1H- |
| | pyrazole-1-carboxamide |
| 821 | N-benzyl-5-cyclopropyl-3-{2-hydroxy-4-[(thien-2-ylcarbonyl)amino]phenyl}-1H- |
| | pyrazole-1-carboxamide |
| 822 | 5-cyclopropyl-3-{2-hydroxy-4-[(thien-2-ylcarbonyl)amino]phenyl}-N-(2- |
| | phenylethyl)-1H-pyrazole-1-carboxamide |
| 823 | 5-cyclopropyl-3-{2-hydroxy-4-[(thien-2-ylcarbonyl)amino]phenyl}-N-(2- |
| | methylbenzyl)-1H-pyrazole-1-carboxamide |
| 824 | 5-cyclopropyl-3-{2-hydroxy-4-[(thien-2-ylcarbonyl)amino]phenyl}-N-(3- |
| | methylbenzyl)-1H-pyrazole-1-carboxamide |
| 825 | 5-cyclopropyl-3-{2-hydroxy-4-[(thien-2-ylcarbonyl)amino]phenyl}-N-(4- |
| | methylbenzyl)-1H-pyrazole-1-carboxamide |
| 826 | N-(2-chlorobenzyl)-5-cyclopropyl-3-{2-hydroxy-4-[(thien-2- |
| | ylcarbonyl)amino]phenyl}-1H-pyrazole-1-carboxamide |
| 827 | 5-cyclopropyl-N-(4-fluorobenzyl)-3-{2-hydroxy-4-[(thien-2- |
| | ylcarbonyl)amino]phenyl}-1H-pyrazole-1-carboxamide |
| 828 | 5-cyclopropyl-N-(3,4-dichlorobenzyl)-3-{2-hydroxy-4-[(thien-2- |
| | ylcarbonyl)amino]phenyl}-1H-pyrazole-1-carboxamide |
| 829 | 5-cyclopropyl-3-{2-hydroxy-4-[(thien-2-ylcarbonyl)amino]phenyl}-N-(4- |
| | methoxybenzyl)-1H-pyrazole-1-carboxamide |
| 830 | N-(sec-butyl)-5-cyclopropyl-3-{2-hydroxy-4-[(thien-2-ylcarbonyl)amino]phenyl}- |
| | 1H-pyrazole-1-carboxamide |
| 831 | 5-cyclopropyl-N-(2-fluorobenzyl)-3-{2-hydroxy-4-[(thien-2- |
| | ylcarbonyl)amino]phenyl}-1H-pyrazole-1-carboxamide |
| 832 | 5-cyclopropyl-N-(3-fluorobenzyl)-3-{2-hydroxy-4-[(thien-2- |
| | ylcarbonyl)amino]phenyl}-1H-pyrazole-1-carboxamide |
| 833 | N,5-dicyclopropyl-3-{2-hydroxy-4-[(thien-2-ylcarbonyl)amino]phenyl}-1H- |
| | pyrazole-1-carboxamide |
| 834 | 5-cyclobutyl-3-{2-hydroxy-4-[(thien-2-ylcarbonyl)amino]phenyl}-N-isopropyl- |
| | 1H-pyrazole-1-carboxamide |
| 835 | 5-cyclobutyl-N-ethyl-3-{2-hydroxy-4-[(thien-2-ylcarbonyl)amino]phenyl}-1H- |
| | pyrazole-1-carboxamide |
| 836 | N-allyl-5-cyclobutyl-3-{2-hydroxy-4-[(thien-2-ylcarbonyl)amino]phenyl}-1H- |
| | pyrazole-1-carboxamide |
| 837 | 5-cyclobutyl-3-{2-hydroxy-4-[(thien-2-ylcarbonyl)amino]phenyl}-N-propyl-1H- |
| | pyrazole-1-carboxamide |
| 838 | N-benzyl-5-cyclobutyl-3-{2-hydroxy-4-[(thien-2-ylcarbonyl)amino]phenyl}-1H- |
| | pyrazole-1-carboxamide |
| 839 | 5-cyclobutyl-3-{2-hydroxy-4-[(thien-2-ylcarbonyl)amino]phenyl}-N-(2- |
| | phenylethyl)-1H-pyrazole-1-carboxamide |
| 840 | 5-cyclobutyl-3-{2-hydroxy-4-[(thien-2-ylcarbonyl)amino]phenyl}-N-(2- |
| | methylbenzyl)-1H-pyrazole-1-carboxamide |
| 841 | 5-cyclobutyl-3-{2-hydroxy-4-[(thien-2-ylcarbonyl)amino]phenyl}-N-(3- |
| | methylbenzyl)-1H-pyrazole-1-carboxamide |
| 842 | 5-cyclobutyl-3-{2-hydroxy-4-[(thien-2-ylcarbonyl)amino]phenyl}-N-(4- |
| | methylbenzyl)-1H-pyrazole-1-carboxamide |
| 843 | N-(2-chlorobenzyl)-5-cyclobutyl-3-{2-hydroxy-4-[(thien-2- |
| | ylcarbonyl)amino]phenyl}-1H-pyrazole-1-carboxamide |
| 844 | 5-cyclobutyl-N-(4-fluorobenzyl)-3-{2-hydroxy-4-[(thien-2- |
| | ylcarbonyl)amino]phenyl}-1H-pyrazole-1-carboxamide |
| 845 | 5-cyclobutyl-N-(3,4-dichlorobenzyl)-3-{2-hydroxy-4-[(thien-2- |
| | ylcarbonyl)amino]phenyl}-1H-pyrazole-1-carboxamide |
| 846 | 5-cyclobutyl-3-{2-hydroxy-4-[(thien-2-ylcarbonyl)amino]phenyl}-N-(4- |
| | methoxybenzyl)-1H-pyrazole-1-carboxamide |
| 847 | N-(sec-butyl)-5-cyclobutyl-3-{2-hydroxy-4-[(thien-2-ylcarbonyl)amino]phenyl}- |
| | 1H-pyrazole-1-carboxamide |
| 848 | 5-cyclobutyl-N-(2-fluorobenzyl)-3-{2-hydroxy-4-[(thien-2- |
| | ylcarbonyl)amino]phenyl}-1H-pyrazole-1-carboxamide |
| 849 | 5-cyclobutyl-N-(3-fluorobenzyl)-3-{2-hydroxy-4-[(thien-2- |
| | ylcarbonyl)amino]phenyl}-1H-pyrazole-1-carboxamide |
| 850 | 5-cyclobutyl-N-cyclopropyl-3-{2-hydroxy-4-[(thien-2-ylcarbonyl)amino]phenyl}- |
| | 1H-pyrazole-1-carboxamide |
| 851 | 5-cyclopentyl-3-{2-hydroxy-4-[(thien-2-ylcarbonyl)amino]phenyl}-N-isopropyl- |
| | 1H-pyrazole-1-carboxamide |
| 852 | 5-cyclopentyl-N-ethyl-3-{2-hydroxy-4-[(thien-2-ylcarbonyl)amino]phenyl}-1H- |
| | pyrazole-1-carboxamide |
| 853 | N-allyl-5-cyclopentyl-3-{2-hydroxy-4-[(thien-2-ylcarbonyl)amino]phenyl}-1H- |
| | pyrazole-1-carboxamide |
| 854 | 5-cyclopentyl-3-{2-hydroxy-4-[(thien-2-ylcarbonyl)amino]phenyl}-N-propyl-1H- |
| | pyrazole-1-carboxamide |
| 855 | N-benzyl-5-cyclopentyl-3-{2-hydroxy-4-[(thien-2-ylcarbonyl)amino]phenyl}-1H- |
| | pyrazole-1-carboxamide |
| 856 | 5-cyclopentyl-3-{2-hydroxy-4-[(thien-2-ylcarbonyl)amino]phenyl}-N-(2- |
| | phenylethyl)-1H-pyrazole-1-carboxamide |
| 857 | 5-cyclopentyl-3-{2-hydroxy-4-[(thien-2-ylcarbonyl)amino]phenyl}-N-(2- |
| | methylbenzyl)-1H-pyrazole-1-carboxamide |
| 858 | 5-cyclopentyl-3-{2-hydroxy-4-[(thien-2-ylcarbonyl)amino]phenyl}-N-(3- |
| | methylbenzyl)-1H-pyrazole-1-carboxamide |
| 859 | 5-cyclopentyl-3-{2-hydroxy-4-[(thien-2-ylcarbonyl)amino]phenyl}-N-(4- |
| | methylbenzyl)-1H-pyrazole-1-carboxamide |
| 860 | N-(2-chlorobenzyl)-5-cyclopentyl-3-{2-hydroxy-4-[(thien-2- |
| | ylcarbonyl)amino]phenyl}-1H-pyrazole-1-carboxamide |
| 861 | 5-cyclopentyl-N-(4-fluorobenzyl)-3-{2-hydroxy-4-[(thien-2- |
| | ylcarbonyl)amino]phenyl}-1H-pyrazole-1-carboxamide |
| 862 | 5-cyclopentyl-N-(3,4-dichlorobenzyl)-3-{2-hydroxy-4-[(thien-2- |
| | ylcarbonyl)amino]phenyl}-1H-pyrazole-1-carboxamide |
| 863 | 5-cyclopentyl-3-{2-hydroxy-4-[(thien-2-ylcarbonyl)amino]phenyl}-N-(4- |
| | methoxybenzyl)-1H-pyrazole-1-carboxamide |
| 864 | N-(sec-butyl)-5-cyclopentyl-3-{2-hydroxy-4-[(thien-2-ylcarbonyl)amino]phenyl}- |
| | 1H-pyrazole-1-carboxamide |
| 865 | 5-cyclopentyl-N-(2-fluorobenzyl)-3-{2-hydroxy-4-[(thien-2- |
| | ylcarbonyl)amino]phenyl}-1H-pyrazole-1-carboxamide |
| 866 | 5-cyclopentyl-N-(3-fluorobenzyl)-3-{2-hydroxy-4-[(thien-2- |
| | ylcarbonyl)amino]phenyl}-1H-pyrazole-1-carboxamide |
| 867 | 5-cyclopentyl-N-cyclopropyl-3-{2-hydroxy-4-[(thien-2- |
| | ylcarbonyl)amino]phenyl}-1H-pyrazole-1-carboxamide |
| 868 | N-(4-{5-cyclopropyl-1-[(isopropylamino)carbonyl]-1H-pyrazol-3-yl}-3- |
| | hydroxyphenyl)quinoxaline-2-carboxamide |
| 869 | N-(4-{5-cyclopropyl-1-[(ethylamino)carbonyl]-1H-pyrazol-3-yl}-3- |
| | hydroxyphenyl)quinoxaline-2-carboxamide |
| 870 | N-(4-{1-[(allylamino)carbonyl]-5-cyclopropyl-1H-pyrazol-3-yl}-3- |
| | hydroxyphenyl)quinoxaline-2-carboxamide |
| 871 | N-(4-{5-cyclopropyl-1-[(propylamino)carbonyl]-1H-pyrazol-3-yl}-3- |
| | hydroxyphenyl)quinoxaline-2-carboxamide |
| 872 | N-(4-{1-[(benzylamino)carbonyl]-5-cyclopropyl-1H-pyrazol-3-yl}-3- |
| | hydroxyphenyl)quinoxaline-2-carboxamide |
| 873 | N-[4-(5-cyclopropyl-1-{[(2-phenylethyl)amino]carbonyl}-1H-pyrazol-3-yl)-3- |
| | hydroxyphenyl]quinoxaline-2-carboxamide |
| 874 | N-[4-(5-cyclopropyl-1-{[(2-methylbenzyl)amino]carbonyl}-1H-pyrazol-3-yl)-3- |
| | hydroxyphenyl]quinoxaline-2-carboxamide |
| 875 | N-[4-(5-cyclopropyl-1-{[(3-methylbenzyl)amino]carbonyl}-1H-pyrazol-3-yl)-3- |
| | hydroxyphenyl]quinoxaline-2-carboxamide |
| 876 | N-[4-(5-cyclopropyl-1-{[(4-methylbenzyl)amino]carbonyl}-1H-pyrazol-3-yl)-3- |
| | hydroxyphenyl]quinoxaline-2-carboxamide |
| 877 | N-[4-(1-{[(2-chlorobenzyl)amino]carbonyl}-5-cyclopropyl-1H-pyrazol-3-yl)-3- |
| | hydroxyphenyl]quinoxaline-2-carboxamide |
| 878 | N-[4-(5-cyclopropyl-1-{[(4-fluorobenzyl)amino]carbonyl}-1H-pyrazol-3-yl)-3- |
| | hydroxyphenyl]quinoxaline-2-carboxamide |
| 879 | N-[4-(5-cyclopropyl-1-{[(3 ,4-dichlorobenzyl)amino]carbonyl}-1H-pyrazol-3-yl)- |
| | 3-hydroxyphenyl]quinoxaline-2-carboxamide |
| 880 | N-[4-(5-cyclopropyl-1-{[(4-methoxybenzyl)amino]carbonyl}-1H-pyrazol-3-yl)-3- |
| | hydroxyphenyl]quinoxaline-2-carboxamide |
| 881 | N-(4-{1-[(sec-butylamino)carbonyl]-5-cyclopropyl-1H-pyrazol-3-yl}-3- |
| | hydroxyphenyl)quinoxaline-2-carboxamide |
| 882 | N-[4-(5-cyclopropyl-1-{[(2-fluorobenzyl)amino]carbonyl}-1H-pyrazol-3-yl)-3- |
| | hydroxyphenyl]quinoxaline-2-carboxamide |
| 883 | N-[4-(5-cyclopropyl-1-{[(3-fluorobenzyl)amino]carbonyl}-1H-pyrazol-3-yl)-3- |
| | hydroxyphenyl]quinoxaline-2-carboxamide |
| 884 | N-(4-{5-cyclopropyl-1-[(cyclopropylamino)carbonyl]-1H-pyrazol-3-yl}-3- |
| | hydroxyphenyl)quinoxaline-2-carboxamide |
| 885 | N-(4-{5-cyclobutyl-1-[(isopropylamino)carbonyl]-1H-pyrazol-3-yl}-3- |
| | hydroxyphenyl)quinoxaline-2-carboxamide |
| 886 | N-(4-{5-cyclobutyl-1-[(ethylamino)carbonyl]-1H-pyrazol-3-yl}-3- |
| | hydroxyphenyl)quinoxaline-2-carboxamide |
| 887 | N-(4-{1-[(allylamino)carbonyl]-5-cyclobutyl-1H-pyrazol-3-yl}-3- |
| | hydroxyphenyl)quinoxaline-2-carboxamide |
| 888 | N-(4-{5-cyclobutyl-1-[(propylamino)carbonyl]-1H-pyrazol-3-yl}-3- |
| | hydroxyphenyl)quinoxaline-2-carboxamide |
| 889 | N-(4-{1-[(benzylamino)carbonyl]-5-cyclobutyl-1H-pyrazol-3-yl}-3- |
| | hydroxyphenyl)quinoxaline-2-carboxamide |
| 890 | N-[4-(5-cyclobutyl-1-{[(2-phenylethyl)amino]carbonyl}-1H-pyrazol-3-yl)-3- |
| | hydroxyphenyl]quinoxaline-2-carboxamide |
| 891 | N-[4-(5-cyclobutyl-1-{[(2-methylbenzyl)amino]carbonyl}-1H-pyrazol-3-yl)-3- |
| | hydroxyphenyl]quinoxaline-2-carboxamide |
| 892 | N-[4-(5-cyclobutyl-1-{[(3-methylbenzyl)amino]carbonyl}-1H-pyrazol-3-yl)-3- |
| | hydroxyphenyl]quinoxaline-2-carboxamide |
| 893 | N-[4-(5-cyclobutyl-1-{[(4-methylbenzyl)amino]carbonyl}-1H-pyrazol-3-yl)-3- |
| | hydroxyphenyl]quinoxaline-2-carboxamide |
| 894 | N-[4-(1-{[(2-chlorobenzyl)amino]carbonyl}-5-cyclobutyl-1H-pyrazol-3-yl)-3- |
| | hydroxyphenyl]quinoxaline-2-carboxamide |
| 895 | N-[4-(5-cyclobutyl-1-{[(4-fluorobenzyl)amino]carbonyl}-1H-pyrazol-3-yl)-3- |
| | hydroxyphenyl]quinoxaline-2-carboxamide |
| 896 | N-[4-(5-cyclobutyl-1-{[(3,4-dichlorobenzyl)amino]carbonyl}-1H-pyrazol-3-yl)-3- |
| | hydroxyphenyl]quinoxaline-2-carboxamide |
| 897 | N-[4-(5-cyclobutyl-1-{[(4-methoxybenzyl)amino]carbonyl}-1H-pyrazol-3-yl)-3- |
| | hydroxyphenyl]quinoxaline-2-carboxamide |
| 898 | N-(4-{1-[(sec-butylamino)carbonyl]-5-cyclobutyl-1H-pyrazol-3-yl}-3- |
| | hydroxyphenyl)quinoxaline-2-carboxamide |
| 899 | N-[4-(5-cyclobutyl-1-{[(2-fluorobenzyl)amino]carbonyl}-1H-pyrazol-3-yl)-3- |
| | hydroxyphenyl]quinoxaline-2-carboxamide |
| 900 | N-[4-(5-cyclobutyl-1-{[(3-fluorobenzyl)amino]carbonyl}-1H-pyrazol-3-yl)-3- |
| | hydroxyphenyl]quinoxaline-2-carboxamide |
| 901 | N-(4-{5-cyclobutyl-1-[(cyclopropylamino)carbonyl]-1H-pyrazol-3-yl}-3- |
| | hydroxyphenyl)quinoxaline-2-carboxamide |
| 902 | N-(4-{5-cyclopentyl-1-[(isopropylamino)carbonyl]-1H-pyrazol-3-yl}-3- |
| | hydroxyphenyl)quinoxaline-2-carboxamide |
| 903 | N-(4-{5-cyclopentyl-1-[(ethylamino)carbonyl]-1H-pyrazol-3-yl}-3- |
| | hydroxyphenyl)quinoxaline-2-carboxamide |
| 904 | N-(4-{1-[(allylamino)carbonyl]-5-cyclopentyl-1H-pyrazol-3-yl}-3- |
| | hydroxyphenyl)quinoxaline-2-carboxamide |
| 905 | N-(4-{5-cyclopentyl-1-[(propylamino)carbonyl]-1H-pyrazol-3-yl}-3- |
| | hydroxyphenyl)quinoxaline-2-carboxamide |
| 906 | N-(4-{1-[(benzylamino)carbonyl]-5-cyclopentyl-1H-pyrazol-3-yl}-3- |
| | hydroxyphenyl)quinoxaline-2-carboxamide |
| 907 | N-[4-(5-cyclopentyl-1-{[(2-phenylethyl)amino]carbonyl}-1H-pyrazol-3-yl)-3- |
| | hydroxyphenyl]quinoxaline-2-carboxamide |
| 908 | N-[4-(5-cyclopentyl-1-{[(2-methylbenzyl)amino]carbonyl}-1H-pyrazol-3-yl)-3- |
| | hydroxyphenyl]quinoxaline-2-carboxamide |
| 909 | N-[4-(5-cyclopentyl-1-{[(3-methylbenzyl)amino]carbonyl}-1H-pyrazol-3-yl)-3- |
| | hydroxyphenyl]quinoxaline-2-carboxamide |
| 910 | N-[4-(5-cyclopentyl-1-{[(4-methylbenzyl)amino]carbonyl}-1H-pyrazol-3-yl)-3- |
| | hydroxyphenyl]quinoxaline-2-carboxamide |
| 911 | N-[4-(1-{[(2-chlorobenzyl)amino]carbonyl}-5-cyclopentyl-1H-pyrazol-3-yl)-3- |
| | hydroxyphenyl]quinoxaline-2-carboxamide |
| 912 | N-[4-(5-cyclopentyl-1-{[(4-fluorobenzyl)amino]carbonyl}-1H-pyrazol-3-yl)-3- |
| | hydroxyphenyl]quinoxaline-2-carboxamide |
| 913 | N-[4-(5-cyclopentyl-1-{[(3,4-dichlorobenzyl)amino]carbonyl}-1H-pyrazol-3-yl)- |
| | 3-hydroxyphenyl]quinoxaline-2-carboxamide |
| 914 | N-[4-(5-cyclopentyl-1-{[(4-methoxybenzyl)amino]carbonyl}-1H-pyrazol-3-yl)-3- |
| | hydroxyphenyl]quinoxaline-2-carboxamide |
| 915 | N-(4-{1-[(sec-butylamino)carbonyl]-5-cyclopentyl-1H-pyrazol-3-yl}-3- |
| | hydroxyphenyl)quinoxaline-2-carboxamide |
| 916 | N-[4-(5-cyclopentyl-1-{[(2-fluorobenzyl)amino]carbonyl}-1H-pyrazol-3-yl)-3- |
| | hydroxyphenyl]quinoxaline-2-carboxamide |
| 917 | N-[4-(5-cyclopentyl-1-{[(3-fluorobenzyl)amino]carbonyl}-1H-pyrazol-3-yl)-3- |
| | hydroxyphenyl]quinoxaline-2-carboxamide |
| 918 | N-(4-{5-cyclopentyl-1-[(cyclopropylamino)carbonyl]-1H-pyrazol-3-yl}-3- |
| | hydroxyphenyl)quinoxaline-2-carboxamide |
| 919 | N-(4-{5-cyclopropyl-1-[(isopropylamino)carbonyl]-1H-pyrazol-3-yl}-3- |
| | hydroxyphenyl)nicotinamide |
| 920 | N-(4-{5-cyclopropyl-1-[(ethylamino)carbonyl]-1H-pyrazol-3-yl}-3- |
| | hydroxyphenyl)nicotinamide |
| 921 | N-(4-{1-[(allylamino)carbonyl]-5-cyclopropyl-1H-pyrazol-3-yl}-3- |
| | hydroxyphenyl)nicotinamide |
| 922 | N-(4-{5-cyclopropyl-1-[(propylamino)carbonyl]-1H-pyrazol-3-yl}-3- |
| | hydroxyphenyl)nicotinamide |
| 923 | N-(4-{1-[(benzylamino)carbonyl]-5-cyclopropyl-1H-pyrazol-3-yl}-3- |
| | hydroxyphenyl)nicotinamide |
| 924 | N-[4-(5-cyclopropyl-1-{[(2-phenylethyl)amino]carbonyl}-1H-pyrazol-3-yl)-3- |
| | hydroxyphenyl]nicotinamide |
| 925 | N-[4-(5-cyclopropyl-1-{[(2-methylbenzyl)amino]carbonyl}-1H-pyrazol-3-yl)-3- |
| | hydroxyphenyl]nicotinamide |
| 926 | N-[4-(5-cyclopropyl-1-{[(3-methylbenzyl)amino]carbonyl}-1H-pyrazol-3-yl)-3- |
| | hydroxyphenyl]nicotinamide |
| 927 | N-[4-(5-cyclopropyl-1-{[(4-methylbenzyl)amino]carbonyl}-1H-pyrazol-3-yl)-3- |
| | hydroxyphenyl]nicotinamide |
| 928 | N-[4-(1-{[(2-chlorobenzyl)amino]carbonyl}-5-cyclopropyl-1H-pyrazol-3-yl)-3- |
| | hydroxyphenyl]nicotinamide |
| 929 | N-[4-(5-cyclopropyl-1-{[(4-fluorobenzyl)amino]carbonyl}-1H-pyrazol-3-yl)-3 - |
| | hydroxyphenyl]nicotinamide |
| 930 | N-[4-(5-cyclopropyl-1-{[(3,4-dichlorobenzyl)amino]carbonyl}-1H-pyrazol-3-yl)- |
| | 3-hydroxyphenyl]nicotinamide |
| 931 | N-[4-(5-cyclopropyl-1-{[(4-methoxybenzyl)amino]carbonyl}-1H-pyrazol-3-yl)-3- |
| | hydroxyphenyl]nicotinamide |
| 932 | N-(4-{1-[(sec-butylamino)carbonyl]-5-cyclopropyl-1H-pyrazol-3-yl}-3- |
| | hydroxyphenyl)nicotinamide |
| 933 | N-[4-(5 -cyclopropyl-1-{[(2-fluorobenzyl)amino]carbonyl}-1H-pyrazol-3-yl)-3- |
| | hydroxyphenyl]nicotinamide |
| 934 | N-[4-(5-cyclopropyl-1-{[(3-fluorobenzyl)amino]carbonyl}-1H-pyrazol-3-yl)-3- |
| | hydroxyphenyl]nicotinamide |
| 935 | N-(4-{5-cyclopropyl-1-[(cyclopropylamino)carbonyl]-1H-pyrazol-3-yl}-3- |
| | hydroxyphenyl)nicotinamide |
| 936 | N-(4-{5-cyclobutyl-1-[(isopropylamino)carbonyl]-1H-pyrazol-3-yl}-3- |
| | hydroxyphenyl)nicotinamide |
| 937 | N-(4-{5-cyclobutyl-1-[(ethylamino)carbonyl]-1H-pyrazol-3-yl}-3- |
| | hydroxyphenyl)nicotinamide |
| 938 | N-(4-{1-[(allylamino)carbonyl]-5-cyclobutyl-1H-pyrazol-3-yl}-3- |
| | hydroxyphenyl)nicotinamide |
| 939 | N-(4-{5-cyclobutyl-1-[(propylamino)carbonyl]-1H-pyrazol-3-yl}-3- |
| | hydroxyphenyl)nicotinamide |
| 940 | N-(4-{1-[(benzylamino)carbonyl]-5-cyclobutyl-1H-pyrazol-3-yl}-3- |
| | hydroxyphenyl)nicotinamide |
| 941 | N-[4-(5-cyclobutyl-1-{[(2-phenylethyl)amino]carbonyl}-1H-pyrazol-3-yl)-3- |
| | hydroxyphenyl]nicotinamide |
| 942 | N-[4-(5-cyclobutyl-1-{[(2-methylbenzyl)amino]carbonyl}-1H-pyrazol-3-yl)-3- |
| | hydroxyphenyl]nicotinamide |
| 943 | N-[4-(5-cyclobutyl-1-{[(3-methylbenzyl)amino]carbonyl}-1H-pyrazol-3-yl)-3- |
| | hydroxyphenyl]nicotinamide |
| 944 | N-[4-(5-cyclobutyl-1-{[(4-methylbenzyl)amino]carbonyl}-1H-pyrazol-3-yl)-3- |
| | hydroxyphenyl]nicotinamide |
| 945 | N-[4-(1-{[(2-chlorobenzyl)amino]carbonyl}-5-cyclobutyl-1H-pyrazol-3-yl)-3- |
| | hydroxyphenyl]nicotinamide |
| 946 | N-[4-(5-cyclobutyl-1-{[(4-fluorobenzyl)amino]carbonyl}-1H-pyrazol-3-yl)-3- |
| | hydroxyphenyl]nicotinamide |
| 947 | N-[4-(5-cyclobutyl-1-{[(3,4-dichlorobenzyl)amino]carbonyl}-1H-pyrazol-3-yl)-3- |
| | hydroxyphenyl]nicotinamide |
| 948 | N-[4-(5-cyclobutyl-1-{[(4-methoxybenzyl)amino]carbonyl}-1H-pyrazol-3-yl)-3- |
| | hydroxyphenyl]nicotinamide |
| 949 | N-(4-{1-[(sec-butylamino)carbonyl]-5-cyclobutyl-1H-pyrazol-3-yl}-3- |
| | hydroxyphenyl)nicotinamide |
| 950 | N-[4-(5-cyclobutyl-1-{[(2-fluorobenzyl)amino]carbonyl}-1H-pyrazol-3-yl)-3- |
| | hydroxyphenyl]nicotinamide |
| 951 | N-[4-(5-cyclobutyl-1-{[(3-fluorobenzyl)amino]carbonyl}-1H-pyrazol-3-yl)-3- |
| | hydroxyphenyl]nicotinamide |
| 952 | N-(4-{5-cyclobutyl-1-[(cyclopropylamino)carbonyl]-1H-pyrazol-3-yl}-3- |
| | hydroxyphenyl)nicotinamide |
| 953 | N-(4-{5-cyclopentyl-1-[(isopropylamino)carbonyl]-1H-pyrazol-3-yl}-3- |
| | hydroxyphenyl)nicotinamide |
| 954 | N-(4-{5-cyclopentyl-1-[(ethylamino)carbonyl]-1H-pyrazol-3-yl}-3- |
| | hydroxyphenyl)nicotinamide |
| 955 | N-(4-{1-[(allylamino)carbonyl]-5-cyclopentyl-1H-pyrazol-3-yl}-3- |
| | hydroxyphenyl)nicotinamide |
| 956 | N-(4-{5-cyclopentyl-1-[(propylamino)carbonyl]-1H-pyrazol-3-yl}-3- |
| | hydroxyphenyl)nicotinamide |
| 957 | N-(4-{1-[(benzylamino)carbonyl]-5-cyclopentyl-1H-pyrazol-3-yl}-3- |
| | hydroxyphenyl)nicotinamide |
| 958 | N-[4-(5-cyclopentyl-1-{[(2-phenylethyl)amino]carbonyl}-1H-pyrazol-3-yl)-3- |
| | hydroxyphenyl]nicotinamide |
| 959 | N-[4-(5-cyclopentyl-1-{[(2-methylbenzyl)amino]carbonyl}-1H-pyrazol-3-yl)-3- |
| | hydroxyphenyl]nicotinamide |
| 960 | N-[4-(5-cyclopentyl-1-{[(3-methylbenzyl)amino]carbonyl}-1H-pyrazol-3-yl)-3- |
| | hydroxyphenyl]nicotinamide |
| 961 | N-[4-(5-cyclopentyl-1-{[(4-methylbenzyl)amino]carbonyl}-1H-pyrazol-3-yl)-3- |
| | hydroxyphenyl]nicotinamide |
| 962 | N-[4-(1-{[(2-chlorobenzyl)amino]carbonyl}-5-cyclopentyl-1H-pyrazol-3-yl)-3- |
| | hydroxyphenyl]nicotinamide |
| 963 | N-[4-(5-cyclopentyl-1-{[(4-fluorobenzyl)amino]carbonyl}-1H-pyrazol-3-yl)-3- |
| | hydroxyphenyl]nicotinamide |
| 964 | N-[4-(5-cyclopentyl-1-{[(3,4-dichlorobenzyl)amino]carbonyl}-1H-pyrazol-3-yl)- |
| | 3-hydroxyphenyl]nicotinamide |
| 965 | N-(4-{1-[(sec-butylamino)carbonyl]-5-cyclopentyl-1H-pyrazol-3-yl}-3- |
| | hydroxyphenyl)nicotinamide |
| 966 | N-(4-{1-[(sec-butylamino)carbonyl]-5-cyclopentyl-1H-pyrazol-3-yl}-3- |
| | hydroxyphenyl)nicotinamide |
| 967 | N-[4-(5-cyclopentyl-1-{[(2-fluorobenzyl)amino]carbonyl}-1H-pyrazol-3-yl)-3- |
| | hydroxyphenyl]nicotinamide |
| 968 | N-[4-(5-cyclopentyl-1-{[(3-fluorobenzyl)amino]carbonyl}-1H-pyrazol-3-yl)-3- |
| | hydroxyphenyl]nicotinamide |
| 969 | N-(4-{5-cyclopentyl-1-[(cyclopropylamino)carbonyl]-1H-pyrazol-3-yl}-3- |
| | hydroxyphenyl)nicotinamide |
| 970 | N-(4-{5-cyclopropyl-1-[(isopropylamino)carbonyl]-1H-pyrazol-3-yl}-3- |
| | hydroxyphenyl)isonicotinamide |
| 971 | N-(4-{5-cyclopropyl-1-[(ethylamino)carbonyl]-1H-pyrazol-3-yl}-3- |
| | hydroxyphenyl)isonicotinamide |
| 972 | N-(4-{1-[(allylamino)carbonyl]-5-cyclopropyl-1H-pyrazol-3-yl}-3- |
| | hydroxyphenyl)isonicotinamide |
| 973 | N-(4-{5-cyclopropyl-1-[(propylamino)carbonyl]-1H-pyrazol-3-yl}-3- |
| | hydroxyphenyl)isonicotinamide |
| 974 | N-(4-{1-[(benzylamino)carbonyl]-5-cyclopropyl-1H-pyrazol-3-yl}-3- |
| | hydroxyphenyl)isonicotinamide |
| 975 | N-[4-(5-cyclopropyl-1-{[(2-phenylethyl)amino]carbonyl}-1H-pyrazol-3-yl)-3- |
| | hydroxyphenyl]isonicotinamide |
| 976 | N-[4-(5-cyclopropyl-1-{[(2-methylbenzyl)amino]carbonyl}-1H-pyrazol-3-yl)-3- |
| | hydroxyphenyl]isonicotinamide |
| 977 | N-[4-(5-cyclopropyl-1-{[(3-methylbenzyl)amino]carbonyl}-1H-pyrazol-3-yl)-3- |
| | hydroxyphenyl]isonicotinamide |
| 978 | N-[4-(5-cyclopropyl-1-{[(4-methylbenzyl)amino]carbonyl}-1H-pyrazol-3-yl)-3- |
| | hydroxyphenyl]isonicotinamide |
| 979 | N-[4-(1-{[(2-chlorobenzyl)amino]carbonyl}-5-cyclopropyl-1H-pyrazol-3-yl)-3- |
| | hydroxyphenyl]isonicotinamide |
| 980 | N-[4-(5-cyclopropyl-1-{[(4-fluorobenzyl)amino]carbonyl}-1H-pyrazol-3-yl)-3- |
| | hydroxyphenyl]isonicotinamide |
| 981 | N-[4-(5-cyclopropyl-1-{[(3,4-dichlorobenzyl)amino]carbonyl}-1H-pyrazol-3-yl)- |
| | 3-hydroxyphenyl]isonicotinamide |
| 982 | N-[4-(5-cyclopropyl-1-{[(4-methoxybenzyl)amino]carbonyl}-1H-pyrazol-3-yl)-3- |
| | hydroxyphenyl]isonicotinamide |
| 983 | N-(4-{1-[(sec-butylamino)carbonyl]-5-cyclopropyl-1H-pyrazol-3-yl}-3- |
| | hydroxyphenyl)isonicotinamide |
| 984 | N-[4-(5-cyclopropyl-1-{[(2-fluorobenzyl)amino]carbonyl}-1H-pyrazol-3-yl)-3- |
| | hydroxyphenyl]isonicotinamide |
| 985 | N-[4-(5-cyclopropyl-1-{[(3-fluorobenzyl)amino]carbonyl}-1H-pyrazol-3-yl)-3- |
| | hydroxyphenyl]isonicotinamide |
| 986 | N-(4-{5-cyclopropyl-1-[(cyclopropylamino)carbonyl]-1H-pyrazol-3-yl}-3- |
| | hydroxyphenyl)isonicotinamide |
| 987 | N-(4-{5-cyclobutyl-1-[(isopropylamino)carbonyl]-1H-pyrazol-3-yl}-3- |
| | hydroxyphenyl)isonicotinamide |
| 988 | N-(4-{5-cyclobutyl-1-[(ethylamino)carbonyl]-1H-pyrazol-3-yl}-3- |
| | hydroxyphenyl)isonicotinamide |
| 989 | N-(4-{1-[(allylamino)carbonyl]-5-cyclobutyl-1H-pyrazol-3-yl}-3- |
| | hydroxyphenyl)isonicotinamide |
| 990 | N-(4-{5-cyclobutyl-1-[(propylamino)carbonyl]-1H-pyrazol-3-yl}-3- |
| | hydroxyphenyl)isonicotinamide |
| 991 | N-(4-{1-[(benzylamino)carbonyl]-5-cyclobutyl-1H-pyrazol-3-yl}-3- |
| | hydroxyphenyl)isonicotinamide |
| 992 | N-[4-(5-cyclobutyl-1-{[(2-methylbenzyl)amino]carbonyl}-1H-pyrazol-3-yl)-3- |
| | hydroxyphenyl]isonicotinamide |
| 993 | N-[4-(5-cyclobutyl-1-{[(2-methylbenzyl)amino]carbonyl}-1H-pyrazol-3-yl)-3- |
| | hydroxyphenyl]isonicotinamide |
| 994 | N-[4-(5-cyclobutyl-1-{[(3-methylbenzyl)amino]carbonyl}-1H-pyrazol-3-yl)-3- |
| | hydroxyphenyl]isonicotinamide |
| 995 | N-[4-(5-cyclobutyl-1-{[(4-methylbenzyl)amino]carbonyl}-1H-pyrazol-3-yl)-3- |
| | hydroxyphenyl]isonicotinamide |
| 996 | N-[4-(1-{[(2-chlorobenzyl)amino]carbonyl}-5-cyclobutyl-1H-pyrazol-3-yl)-3- |
| | hydroxyphenyl]isonicotinamide |
| 997 | N-[4-(5-cyclobutyl-1-{[(4-fluorobenzyl)amino]carbonyl}-1H-pyrazol-3-yl)-3- |
| | hydroxyphenyl]isonicotinamide |
| 998 | N-[4-(5-cyclobutyl-1-{[(3,4-dichlorobenzyl)amino]carbonyl}-1H-pyrazol-3-yl)-3- |
| | hydroxyphenyl]isonicotinamide |
| 999 | N-[4-(5-cyclobutyl-1-{[(4-methoxybenzyl)amino]carbonyl}-1H-pyrazol-3-yl)-3- |
| | hydroxyphenyl]isonicotinamide |
| 1000 | N-(4-{1-[(sec-butylamino)carbonyl]-5-cyclobutyl-1H-pyrazol-3-yl}-3- |
| | hydroxyphenyl)isonicotinamide |
| 1001 | N-[4-(5-cyclobutyl-1-{[(2-fluorobenzyl)amino]carbonyl}-1H-pyrazol-3-yl)-3- |
| | hydroxyphenyl]isonicotinamide |
| 1002 | N-[4-(5-cyclobutyl-1-{[(3-fluorobenzyl)amino]carbonyl}-1H-pyrazol-3-yl)-3- |
| | hydroxyphenyl]isonicotinamide |
| 1003 | N-(4-{5-cyclobutyl-1-[(cyclopropylamino)carbonyl]-1H-pyrazol-3-yl}-3- |
| | hydroxyphenyl)isonicotinamide |
| 1004 | N-(4-{5-cyclopentyl-1-[(isopropylamino)carbonyl]-1H-pyrazol-3-yl}-3- |
| | hydroxyphenyl)isonicotinamide |
| 1005 | N-(4-{5-cyclopentyl-1-[(ethylamino)carbonyl]-1H-pyrazol-3-yl}-3- |
| | hydroxyphenyl)isonicotinamide |
| 1006 | N-(4-{1-[(allylamino)carbonyl]-5-cyclopentyl-1H-pyrazol-3-yl}-3- |
| | hydroxyphenyl)isonicotinamide |
| 1007 | N-(4-{5-cyclopentyl-1-[(propylamino)carbonyl]-1H-pyrazol-3-yl}-3- |
| | hydroxyphenyl)isonicotinamide |
| 1008 | N-(4-{1-[(benzylamino)carbonyl]-5-cyclopentyl-1H-pyrazol-3-yl}-3- |
| | hydroxyphenyl)isonicotinamide |
| 1009 | N-[4-(5-cyclopentyl-1-{[(2-phenylethyl)amino]carbonyl}-1H-pyrazol-3-yl)-3- |
| | hydroxyphenyl]isonicotinamide |
| 1010 | N-[4-(5-cyclopentyl-1-{[(2-methylbenzyl)amino]carbonyl}-1H-pyrazol-3-yl)-3- |
| | hydroxyphenyl]isonicotinamide |
| 1011 | N-[4-(5-cyclopentyl-1-{[(3-methylbenzyl)amino]carbonyl}-1H-pyrazol-3-yl)-3- |
| | hydroxyphenyl]isonicotinamide |
| 1012 | N-[4-(5-cyclopentyl-1-{[(4-methylbenzyl)amino]carbonyl}-1H-pyrazol-3-yl)-3- |
| | hydroxyphenyl]isonicotinamide |
| 1013 | N-[4-(1-{[(2-chlorobenzyl)amino]carbonyl}-5-cyclopentyl-1H-pyrazol-3-yl)-3- |
| | hydroxyphenyl]isonicotinamide |
| 1014 | N-[4-(5-cyclopentyl-1-{[(4-fluorobenzyl)amino]carbonyl}-1H-pyrazol-3-yl)-3- |
| | hydroxyphenyl]isonicotinamide |
| 1015 | N-[4-(5-cyclopentyl-1-{[(3,4-dichlorobenzyl)amino]carbonyl}-1H-pyrazol-3-yl)- |
| | 3-hydroxyphenyl]isonicotinamide |
| 1016 | N-[4-(5-cyclopentyl-1-{[(4-methoxybenzyl)amino]carbonyl}-1H-pyrazol-3-yl)-3- |
| | hydroxyphenyl]isonicotinamide |
| 1017 | N-(4-{1-[(sec-butylamino)carbonyl]-5-cyclopentyl-1H-pyrazol-3-yl}-3- |
| | hydroxyphenyl)isonicotinamide |
| 1018 | N-[4-(5-cyclopentyl-1-{[(2-fluorobenzyl)amino]carbonyl}-1H-pyrazol-3-yl)-3- |
| | hydroxyphenyl]isonicotinamide |
| 1019 | N-[4-(5-cyclopentyl-1-{[(3-fluorobenzyl)amino]carbonyl}-1H-pyrazol-3-yl)-3- |
| | hydroxyphenyl]isonicotinamide |
| 1020 | N-(4-{5-cyclopentyl-1-[(cyclopropylamino)carbonyl]-1H-pyrazol-3-yl}-3- |
| | hydroxyphenyl)isonicotinamide |
| 1021 | 3-{4-[(1,3-benzodioxol-5-ylcarbonyl)amino]-2-hydroxyphenyl}-5-cyclopropyl-N- |
| | isopropyl-1H-pyrazole-1-carboxamide |
| 1022 | 3-{4-[(1,3-benzodioxol-5-ylcarbonyl)amino]-2-hydroxyphenyl}-5-cyclopropyl-N- |
| | ethyl-1H-pyrazole-1-carboxamide |
| 1023 | N-allyl-3-{4-[(1,3-benzodioxol-5-ylcarbonyl)amino]-2-hydroxyphenyl}-5- |
| | cyclopropyl-1H-pyrazole-1-carboxamide |
| 1024 | 3-{4-[(1,3-benzodioxol-5-ylcarbonyl)amino]-2-hydroxyphenyl}-5-cyclopropyl-N- |
| | propyl-1H-pyrazole-1-carboxamide |
| 1025 | 3-{4-[(1,3-benzodioxol-5-ylcarbonyl)amino]-2-hydroxyphenyl}-N-benzyl-5- |
| | cyclopropyl-1H-pyrazole-1-carboxamide |
| 1026 | 3-{4-[(1,3-benzodioxol-5-ylcarbonyl)amino]-2-hydroxyphenyl}-5-cyclopropyl-N- |
| | (2-phenylethyl)-1H-pyrazole-1-carboxamide |
| 1027 | 3-{4-[(1,3-benzodioxol-5-ylcarbonyl)amino]-2-hydroxyphenyl}-5-cyclopropyl-N- |
| | (2-methylbenzyl)-1H-pyrazole-1-carboxamide |
| 1028 | 3-{4-[(1,3-benzodioxol-5-ylcarbonyl)amino]-2-hydroxyphenyl}-5-cyclopropyl-N- |
| | (3-methylbenzyl)-1H-pyrazole-1-carboxamide |
| 1029 | 3-{4-[(1,3-benzodioxol-5-ylcarbonyl)amino]-2-hydroxyphenyl}-5-cyclopropyl-N- |
| | (4-methylbenzyl)-1H-pyrazole-1-carboxamide |
| 1030 | 3-{4-[(1,3-benzodioxol-5-ylcarbonyl)amino]-2-hydroxyphenyl}-N-(2- |
| | chlorobenzyl)-5-cyclopropyl-1H-pyrazole-1-carboxamide |
| 1031 | 3-{4-[(1,3-benzodioxol-5-ylcarbonyl)amino]-2-hydroxyphenyl}-5-cyclopropyl-N- |
| | (4-fluorobenzyl)-1H-pyrazole-1-carboxamide |
| 1032 | 3-{4-[(1,3-benzodioxol-5-ylcarbonyl)amino]-2-hydroxyphenyl}-5-cyclopropyl-N- |
| | (3,4-dichlorobenzyl)-1H-pyrazole-1-carboxamide |
| 1033 | 3-{4-[(1,3-benzodioxol-5-ylcarbonyl)amino]-2-hydroxyphenyl}-5-cyclopropyl-N- |
| | (4-methoxybenzyl)-1H-pyrazole-1-carboxamide |
| 1034 | 3-{4-[(1,3-benzodioxol-5-ylcarbonyl)amino]-2-hydroxyphenyl}-N-(sec-butyl)-5- |
| | cyclopropyl-1H-pyrazole-1-carboxamide |
| 1035 | 3-{4-[(1,3-benzodioxol-5-ylcarbonyl)amino]-2-hydroxyphenyl}-5-cyclopropyl-N- |
| | (2-fluorobenzyl)-1H-pyrazole-1-carboxamide |
| 1036 | 3-{4-[(1,3-benzodioxol-5-ylcarbonyl)amino]-2-hydroxyphenyl}-5-cyclopropyl-N- |
| | (3-fluorobenzyl)-1H-pyrazole-1-carboxamide |
| 1037 | 3-{4-[(1,3-benzodioxol-5-ylcarbonyl)amino]-2-hydroxyphenyl}-N,5- |
| | dicyclopropyl-1H-pyrazole-1-carboxamide |
| 1038 | 3-{4-[(1,3-benzodioxol-5-ylcarbonyl)amino]-2-hydroxyphenyl}-5-cyclobutyl-N- |
| | isopropyl-1H-pyrazole-1-carboxamide |
| 1039 | 3-{4-[(1,3-benzodioxol-5-ylcarbonyl)amino]-2-hydroxyphenyl}-5-cyclobutyl-N- |
| | ethyl-1H-pyrazole-1-carboxamide |
| 1040 | N-allyl-3-{4-[(1,3-benzodioxol-5-ylcarbonyl)amino]-2-hydroxyphenyl}-5- |
| | cyclobutyl-1H-pyrazole-1-carboxamide |
| 1041 | 3-{4-[(1,3-benzodioxol-5-ylcarbonyl)amino]-2-hydroxyphenyl}-5-cyclobutyl-N- |
| | propyl-1H-pyrazole-1-carboxamide |
| 1042 | 3-{4[(1,3-benzodioxol-5-ylcarbonyl)amino]-2-hydroxyphenyl}-N-benzyl-5- |
| | cyclobutyl-1H-pyrazole-1-carboxamide |
| 1043 | 3-{4-[(1,3-benzodioxol-5-ylcarbonyl)amino]-2-hydroxyphenyl}-5-cyclobutyl-N- |
| | (2-phenylethyl)-1H-pyrazole-1-carboxamide |
| 1044 | 3-{4-[(1,3-benzodioxol-5-ylcarbonyl)amino]-2-hydroxyphenyl}-5-cyclobutyl-N- |
| | (2-methylbenzyl)-1H-pyrazole-1-carboxamide |
| 1045 | 3-{4-[(1,3-benzodioxol-5-ylcarbonyl)amino]-2-hydroxyphenyl}-5-cyclobutyl-N- |
| | (3-methylbenzyl)-1H-pyrazole-1-carboxamide |
| 1046 | 3-{4-[(1,3-benzodioxol-5-ylcarbonyl)amino]-2-hydroxyphenyl}-5-cyclobutyl-N- |
| | (4-methylbenzyl)-1H-pyrazole-1-carboxamide |
| 1047 | 3-{4-[(1,3-benzodioxol-5-ylcarbonyl)amino]-2-hydroxyphenyl}-N-(2- |
| | chlorobenzyl)-5-cyclobutyl-1H-pyrazole-1-carboxamide |
| 1048 | 3-{4-[(1,3-benzodioxol-5-ylcarbonyl)amino]-2-hydroxyphenyl}-5-cyclobutyl-N- |
| | (4-fluorobenzyl)-1H-pyrazole-1-carboxamide |
| 1049 | 3-{4-[(1,3-benzodioxol-5-ylcarbonyl)amino]-2-hydroxyphenyl}-5-cyclobutyl-N- |
| | (3,4-dichlorobenzyl)-1H-pyrazole-1-carboxamide |
| 1050 | 3-{4-[(1,3-benzodioxol-5-ylcarbonyl)amino]-2-hydroxyphenyl}-5-cyclobutyl-N- |
| | (4-methoxybenzyl)-1H-pyrazole-1-carboxamide |
| 1051 | 3-{4-[(1,3-benzodioxol-5-ylcarbonyl)amino]-2-hydroxyphenyl}-N-(sec-butyl)-5- |
| | cyclobutyl-1H-pyrazole-1-carboxamide |
| 1052 | 3-{4-[(1,3-benzodioxol-5-ylcarbonyl)amino]-2-hydroxyphenyl}-5-cyclobutyl-N- |
| | (2-fluorobenzyl)-1H-pyrazole-1-carboxamide |
| 1053 | 3-{4-[(1,3-benzodioxol-5-ylcarbonyl)amino]-2-hydroxyphenyl}-5-cyclobutyl-N- |
| | (3-fluorobenzyl)-1H-pyrazole-1-carboxamide |
| 1054 | 3-{4-[(1,3-benzodioxol-5-ylcarbonyl)amino]-2-hydroxphenyl}-5-cyclobutyl-N- |
| | cyclopropyl-1H-pyrazole-1-carboxamide |
| 1055 | 3-{4-[(1,3-benzodioxol-5-ylcarbonyl)amino]-2-hydroxyphenyl}-5-cyclopentyl-N- |
| | isopropyl-1H-pyrazole-1-carboxamide |
| 1056 | 3-{4-[(1,3-benzodioxol-5-ylcarbonyl)amino]-2-hydroxyphenyl}-5-cyclopentyl-N- |
| | ethyl-1H-pyrazole-1-carboxamide |
| 1057 | N-allyl-3-{4-[(1,3-benzodioxol-5-ylcarbonyl)amino]-2-hydroxyphenyl}-5- |
| | cyclopentyl-1H-pyrazole-1-carboxamide |
| 1058 | 3-{4-[(1,3-benzodioxol-5-ylcarbonyl)amino]-2-hydroxyphenyl}-5-cyclopentyl-N- |
| | propyl-1H-pyrazole-1-carboxamide |
| 1059 | 3-{4-[(1,3-benzodioxol-5-ylcarbonyl)amino]-2-hydroxyphenyl}-N-benzyl-5- |
| | cyclopentyl-1H-pyrazole-1-carboxamide |
| 1060 | 3-{4-[(1,3-benzodioxol-5-ylcarbonyl)amino]-2-hydroxyphenyl}-5-cyclopentyl-N- |
| | (2-phenylethyl)-1H-pyrazole-1-carboxamide |
| 1061 | 3-{4-[(1,3-benzodioxol-5-ylcarbonyl)amino]-2-hydroxyphenyl}-5-cyclopentyl-N- |
| | (2-methylbenzyl)-1H-pyrazole-1-carboxamide |
| 1062 | 3-{4-[(1,3-benzodioxol-5-ylcarbonyl)amino]-2-hydroxyphenyl}-5-cyclopentyl-N- |
| | (3-methylbenzyl)-1H-pyrazole-1-carboxamide |
| 1063 | 3-{4-[(1,3-benzodioxol-5-ylcarbonyl)amino]-2-hydroxyphenyl}-5-cyclopentyl-N- |
| | (4-methylbenzyl)-1H-pyrazole-1-carboxamide |
| 1064 | 3-{4-[(1,3-benzodioxol-5-ylcarbonyl)amino]-2-hydroxyphenyl}-N-(2- |
| | chlorobenzyl-5-cyclopentyl-1H-pyrazole-1-carboxamide |
| 1065 | 3-{4-[(1,3-benzodioxol-5-ylcarbonyl)amino]-2-hydroxyphenyl}-5-cyclopentyl-N- |
| | (4-fluorobenzyl)-1H-pyrazole-1-carboxamide |
| 1066 | 3-{4-[(1,3-benzodioxol-5-ylcarbonyl)amino]-2-hydroxyphenyl}-5-cyclopentyl-N- |
| | (3,4-dichlorobenzyl)-1H-pyrazole-1-carboxamide |
| 1067 | 3-{4-[(1,3-benzodioxol-5-ylcarbonyl)amino]-2-hydroxyphenyl}-5-cyclopentyl-N- |
| | (4-methoxybenzyl)-1H-pyrazole-1-carboxamide |
| 1068 | 3-{4-[(1,3-benzodioxol-5-ylcarbonyl)amino]-2-hydroxyphenyl}-N-(sec-butyl)-5- |
| | cyclopentyl-1H-pyrazole-1-carboxamide |
| 1069 | 3-{4-[(1,3-benzodioxol-5-ylcarbonyl)amino]-2-hydroxyphenyl}-5-cyclopentyl-N- |
| | (2-fluorobenzyl)-1H-pyrazole-1-carboxamide |
| 1070 | 3-{4-[(1,3-benzodioxol-5-ylcarbonyl)amino]-2-hydroxyphenyl}-5-cyclopentyl-N- |
| | (3-fluorobenzyl)-1H-pyrazole-1-carboxamide |
| 1071 | 3-{4-[(1,3-benzodioxol-5-ylcarbonyl)amino]-2-hydroxyphenyl}-5-cyclopentyl-N- |
| | cyclopropyl-1H-pyrazole-1-carboxamide |
| 1072 | 5-cyclopropyl-3-{4-[(2-ethylbutanoyl)amino]-2-hydroxyphenyl}-N-isopropyl-1H- |
| | pyrazole-1-carboxamide |
| 1073 | 5-cyclopropyl-N-ethyl-3-{4-[(2-ethylbutanoyl)amino]-2-hydroxyphenyl}-1H- |
| | pyrazole-1-carboxamide |
| 1074 | N-allyl-5-cyclopropyl-3-{4-[(2-ethylbutanoyl)amino]-2-hydroxyphenyl}-1H- |
| | pyrazole-1-carboxamide |
| 1075 | 5-cyclopropyl-3-{4-[(2-ethylbutanoyl)amino]-2-hydroxyphenyl}-N-propyl-1H- |
| | pyrazole-1-carboxamide |
| 1076 | N-benzyl-5-cyclopropyl-3-{4-[(2-ethylbutanoyl)amino]-2-hydroxyphenyl}-1H- |
| | pyrazole-1-carboxamide |
| 1077 | 5-cyclopropyl-3-{4-[(2-ethylbutanoyl)amino]-2-hydroxyphenyl}-N-(2- |
| | phenylethyl)-1H-pyrazole-1-carboxamide |
| 1078 | 5-cyclopropyl-3-{4-[(2-ethylbutanoyl)amino]-2-hydroxyphenyl}-N-(2- |
| | methylbenzyl)-1H-pyrazole-1-carboxamide |
| 1079 | 5-cyclopropyl-3-{4-[(2-ethylbutanoyl)amino]-2-hydroxyphenyl}-N-(3- |
| | methylbenzyl)-1H-pyrazole-1-carboxamide |
| 1080 | 5-cyclopropyl-3-{4-[(2-ethylbutanoyl)amino]-2-hydroxyphenyl}-N-(4- |
| | methylbenzyl)-1H-pyrazole-1-carboxamide |
| 1081 | N-(2-chlorobenzyl)-5-cyclopropyl-3-{4-[(2-ethylbutanoyl)amino]-2- |
| | hydroxyphenyl}-1H-pyrazole-1-carboxamide |
| 1082 | 5-cyclopropyl-3-{4-[(2-ethylbutanoyl)amino]-2-hydroxyphenyl}-N-(4- |
| | fluorobenzyl)-1H-pyrazole-1-carboxamide |
| 1083 | 5-cyclopropyl-N-(3,4-dichlorobenzyl)-3-{4-[(2-ethylbutanoyl)amino]-2- |
| | hydroxyphenyl}-1H-pyrazole-1-carboxamide |
| 1084 | 5-cyclopropyl-3-{4-[(2-ethylbutanoyl)amino]-2-hydroxyphenyl}-N-(4- |
| | methoxybenzyl)-1H-pyrazole-1-carboxamide |
| 1085 | N-(sec-butyl)-5-cyclopropyl-3-{4-[(2-ethylbutanoyl)amino]-2-hydroxyphenyl}- |
| | 1H-pyrazole-1-carboxamide |
| 1086 | 5-cyclopropyl-3-{4-[(2-ethylbutanoyl)amino]-2-hydroxyphenyl}-N-(2- |
| | fluorobenzyl)-1H-pyrazole-1-carboxamide |
| 1087 | 5-cyclopropyl-3-{4-[(2-ethylbutanoyl)amino]-2-hydroxyphenyl}-N-(3- |
| | fluorobenzyl)-1H-pyrazole-1-carboxamide |
| 1088 | N,5-dicyclopropyl-3-{4-[(2-ethylbutanoyl)amino]-2-hydroxyphenyl}-1H- |
| | pyrazole-1-carboxamide |
| 1089 | 5-cyclobutyl-3-{4-[(2-ethylbutanoyl)amino]-2-hydroxyphenyl}-N-isopropyl-1H- |
| | pyrazole-1-carboxamide |
| 1090 | 5-cyclobutyl-N-ethyl-3-{4-[(2-ethylbutanoyl)amino]-2-hydroxyphenyl}-1H- |
| | pyrazole-1-carboxamide |
| 1091 | N-allyl-5-cyclobutyl-3-{4-[(2-ethylbutanoyl)amino]-2-hydroxyphenyl}-1H- |
| | pyrazole-1-carboxamide |
| 1092 | 5-cyclobutyl-3-{4-[(2-ethylbutanoyl)amino]-2-hydroxyphenyl}-N-propyl-1H- |
| | pyrazole-1-carboxamide |
| 1093 | N-benzyl-5-cyclobutyl-3-{4-[(2-ethylbutanoyl)amino]-2-hydroxyphenyl}-1H- |
| | pyrazole-1-carboxamide |
| 1094 | 5-cyclobutyl-3-{4-[(2-ethylbutanoyl)amino]-2-hydroxyphenyl}-N-(2-phenylethyl)- |
| | 1H-pyrazole-1-carboxamide |
| 1095 | 5-cyclobutyl-3-{4-[(2-ethylbutanoyl)amino]-2-hydroxyphenyl}-N-(2- |
| | methylbenzyl)-1H-pyrazole-1-carboxamide |
| 1096 | 5-cyclobutyl-3-{4-[(2-ethylbutanoyl)amino]-2-hydroxyphenyl}-N-(3- |
| | methylbenzyl)-1H-pyrazole-1-carboxamide |
| 1097 | 5-cyclobutyl-3-{4-[(2-ethylbutanoyl)amino]-2-hydroxyphenyl}-N-(4- |
| | methylbenzyl)-1H-pyrazole-1-carboxamide |
| 1098 | N-(2-chlorobenzyl)-5-cyclobutyl-3-{4-[(2-ethylbutanoyl)amino]-2- |
| | hydroxyphenyl}-1H-pyrazole-1-carboxamide |
| 1099 | 5-cyclobutyl-3-{4-[(2-ethylbutanoyl)amino]-2-hydroxyphenyl}-N-(4- |
| | fluorobenzyl)-1H-pyrazole-1-carboxamide |
| 1100 | 5-cyclobutyl-N-(3,4-dichlorobenzyl)-3-{4-[(2-ethylbutanoyl)amino]-2- |
| | hydroxyphenyl}-1H-pyrazole-1-carboxamide |
| 1101 | 5-cyclobutyl-3-{4-[(2-ethylbutanoyl)amino]-2-hydroxyphenyl}-N-(4- |
| | methoxybenzyl)-1H-pyrazole-1-carboxamide |
| 1102 | N-(sec-butyl)-5-cyclobutyl-3-{4-[(2-ethylbutanoyl)amino]-2-hydroxyphenyl}-1H- |
| | pyrazole-1-carboxamide |
| 1103 | 5-cyclobutyl-3-{4-[(2-ethylbutanoyl)amino]-2-hydroxyphenyl}-N-(2- |
| | fluorobenzyl)-1H-pyrazole-1-carboxamide |
| 1104 | 5-cyclobutyl-3-{4-[(2-ethylbutanoyl)amino]-2-hydroxyphenyl}-N-(3- |
| | fluorobenzyl)-1H-pyrazole-1-carboxamide |
| 1105 | 5-cyclobutyl-N-cyclopropyl-3-{4-[(2-ethylbutanoyl)amino]-2-hydroxyphenyl}- |
| | 1H-pyrazole-1-carboxamide |
| 1106 | 5-cyclopentyl-3-{4-[(2-ethylbutanoyl)amino]-2-hydroxyphenyl}-N-isopropyl-1H- |
| | pyrazole-1-carboxamide |
| 1107 | 5-cyclopentyl-N-ethyl-3-{4-[(2-ethylbutanoyl)amino]-2-hydroxyphenyl}-1H- |
| | pyrazole-1-carboxamide |
| 1108 | N-allyl-5-cyclopentyl-3-{4-[(2-ethylbutanoyl)amino]-2-hydroxyphenyl}-1H- |
| | pyrazole-1-carboxamide |
| 1109 | 5-cyclopentyl-3-{4-[(2-ethylbutanoyl)amino]-2-hydroxyphenyl}-N-propyl-1H- |
| | pyrazole-1-carboxamide |
| 1110 | N-benzyl-5-cyclopentyl-3-{4-[(2-ethylbutanoyl)amino]-2-hydroxyphenyl}-1H- |
| | pyrazole-1-carboxamide |
| 1111 | 5-cyclopentyl-3-{4-[(2-ethylbutanoyl)amino]-2-hydroxyphenyl}-N-(2- |
| | phenylethyl)-1H-pyrazole-1-carboxamide |
| 1112 | 5-cyclopentyl-3-{4-[(2-ethylbutanoyl)amino]-2-hydroxyphenyl}-N-(2- |
| | methylbenzyl)-1H-pyrazole-1-carboxamide |
| 1113 | 5-cyclopentyl-3-{4-[(2-ethylbutanoyl)amino]-2-hydroxyphenyl}-N-(3- |
| | methylbenzyl)-1H-pyrazole-1-carboxamide |
| 1114 | 5-cyclopentyl-3-{4-[(2-ethylbutanoyl)amino]-2-hydroxyphenyl}-N-(4- |
| | methylbenzyl)-1H-pyrazole-1-carboxamide |
| 1115 | N-(2-chlorobenzyl)-5-cyclopentyl-3-{4-[(2-ethylbutanoyl)amino]-2- |
| | hydroxyphenyl}-1H-pyrazole-1-carboxamide |
| 1116 | 5-cyclopentyl-3-{4-[(2-ethylbutanoyl)amino]-2-hydroxyphenyl}-N-(4- |
| | fluorobenzyl)-1H-pyrazole-1-carboxamide |
| 1117 | 5-cyclopentyl-N-(3,4-dichlorobenzyl)-3-{4-[(2-ethylbutanoyl)amino]-2- |
| | hydroxyphenyl}-1H-pyrazole-1-carboxamide |
| 1118 | 5-cyclopentyl-3-{4-[(2-ethylbutanoyl)amino]-2-hydroxyphenyl}-N-(4- |
| | methoxybenzyl)-1H-pyrazole-1-carboxamide |
| 1119 | N-(sec-butyl)-5-cyclopentyl-3-{4-[(2-ethylbutanoyl)amino]-2-hydroxyphenyl}- |
| | 1H-pyrazole-1-carboxamide |
| 1120 | 5-cyclopentyl-3-{4-[(2-ethylbutanoyl)amino]-2-hydroxyphenyl}-N-(2- |
| | fluorobenzyl)-1H-pyrazole-1-carboxamide |
| 1121 | 5-cyclopentyl-3-{4-[(2-ethylbutanoyl)amino]-2-hydroxyphenyl}-N-(3- |
| | fluorobenzyl)-1H-pyrazole-1-carboxamide |
| 1122 | 5-cyclopentyl-N-cyclopropyl-3-{4-[(2-ethylbutanoyl)amino]-2-hydroxyphenyl}- |
| | 1H-pyrazole-1-carboxamide |
| 1123 | 3-{4-[(1-benzothien-2-ylcarbonyl)amino]-2-hydroxyphenyl}-5-cyclopropyl-N- |
| | isopropyl-1H-pyrazole-1-carboxamide |
| 1124 | 3-{4-[(1-benzothien-2-ylcarbonyl)amino]-2-hydroxyphenyl}-5-cyclopropyl-N- |
| | ethyl-1H-pyrazole-1-carboxamide |
| 1125 | N-allyl-3-{4-[(1-benzothien-2-ylcarbonyl)amino]-2-hydroxyphenyl}-5- |
| | cyclopropyl-1H-pyrazole-1-carboxamide |
| 1126 | 3-{4-[(1-benzothien-2-ylcarbonyl)amino]-2-hydroxyphenyl}-5-cyclopropyl-N- |
| | propyl-1H-pyrazole-1-carboxamide |
| 1127 | 3-{4-[(1-benzothien-2-ylcarbonyl)amino]-2-hydroxyphenyl}-N-benzyl-5- |
| | cyclopropyl-1H-pyrazole-1-carboxamide |
| 1128 | 3-{4-[(1-benzothien-2-ylcarbonyl)amino]-2-hydroxyphenyl}-5-cyclopropyl-N-(2- |
| | phenylethyl)-1H-pyrazole-1-carboxamide |
| 1129 | 3-{4-[(1-benzothien-2-ylcarbonyl)amino]-2-hydroxyphenyl}-5-cyclopropyl-N-(2- |
| | methylbenzyl)-1H-pyrazole-1-carboxamide |
| 1130 | 3-{4-[(1-benzothien-2-ylcarbonyl)amino]-2-hydroxyphenyl}-5-cyclopropyl-N-(3- |
| | methylbenzyl)-1H-pyrazole-1-carboxamide |
| 1131 | 3-{4-[(1-benzothien-2-ylcarbonyl)amino]-2-hydroxyphenyl}-5-cyclopropyl-N-(4- |
| | methylbenzyl)-1H-pyrazole-1-carboxamide |
| 1132 | 3-{4-[(1-benzothien-2-ylcarbonyl)amino]-2-hydroxyphenyl}-N-(2-chlorobenzyl)- |
| | 5-cyclopropyl-1H-pyrazole-1-carboxamide |
| 1133 | 3-{4-[(1-benzothien-2-ylcarbonyl)amino]-2-hydroxyphenyl}-5-cyclopropyl-N-(4- |
| | fluorobenzyl)-1H-pyrazole-1-carboxamide |
| 1134 | 3-{4-[(1-benzothien-2-ylcarbonyl)amino]-2-hydroxyphenyl}-5-cyclopropyl-N- |
| | (3,4-dichlorobenzyl)-1H-pyrazole-1-carboxamide |
| 1135 | 3-{4-[(1-benzothien-2-ylcarbonyl)amino]-2-hydroxyphenyl}-5-cyclopropyl-N-(4- |
| | methoxybenzyl)-1H-pyrazole-1-carboxamide |
| 1136 | 3-{4-[(1-benzothien-2-ylcarbonyl)amino]-2-hydroxyphenyl}-N-(sec-butyl)-5- |
| | cyclopropyl-1H-pyrazole-1-carboxamide |
| 1137 | 3-{4-[(1-benzothien-2-ylcarbonyl)amino]-2-hydroxyphenyl}-5-cyclopropyl-N-(2- |
| | fluorobenzyl)-1H-pyrazole-1-carboxamide |
| 1138 | 3-{4-[(1-benzothien-2-ylcarbonyl)amino]-2-hydroxyphenyl}-5-cyclopropyl-N-(3- |
| | fluorobenzyl)-1H-pyrazole-1-carboxamide |
| 1139 | 3-{4-[(1-benzothien-2-ylcarbonyl)amino]-2-hydroxyphenyl}-N,5-dicyclopropyl- |
| | 1H-pyrazole-1-carboxamide |
| 1140 | 3-{4-[(1-benzothien-2-ylcarbonyl)amino]-2-hydroxyphenyl}-5-cyclobutyl-N- |
| | isopropyl-1H-pyrazole-1-carboxamide |
| 1141 | 3-{4-[(1-benzothien-2-ylcarbonyl)amino]-2-hydroxyphenyl}-5-cyclobutyl-N- |
| | ethyl-1H-pyrazole-1-carboxamide |
| 1142 | N-allyl-3-{4-[(1-benzothien-2-ylcarbonyl)amino]-2-hydroxyphenyl}-5-cyclobutyl- |
| | 1H-pyrazole-1-carboxamide |
| 1143 | 3-{4-[(1-benzothien-2-ylcarbonyl)amino]-2-hydroxyphenyl}-5-cyclobutyl-N- |
| | propyl-1H-pyrazole-1-carboxamide |
| 1144 | 3-{4-[(1-benzothien-2-ylcarbonyl)amino]-2-hydroxyphenyl}-N-benzyl-5- |
| | cyclobutyl-1H-pyrazole-1-carboxamide |
| 1145 | 3-{4-[(1-benzothien-2-ylcarbonyl)amino]-2-hydroxyphenyl}-5-cyclobutyl-N-(2- |
| | phenylethyl)-1H-pyrazole-1-carboxamide |
| 1146 | 3-{4-[(1-benzothien-2-ylcarbonyl)amino]-2-hydroxyphenyl}-5-cyclobutyl-N-(2- |
| | methylbenzyl)-1H-pyrazole-1-carboxamide |
| 1147 | 3-{4-[(1-benzothien-2-ylcarbonyl)amino]-2-hydroxyphenyl}-5-cyclobutyl-N-(3- |
| | methylbenzyl)-1H-pyrazole-1-carboxamide |
| 1148 | 3-{4-[(1-benzothien-2-ylcarbonyl)amino]-2-hydroxyphenyl}-5-cyclobutyl-N-(4- |
| | methylbenzyl)-1H-pyrazole-1-carboxamide |
| 1149 | 3-{4-[(1-benzothien-2-ylcarbonyl)amino]-2-hydroxyphenyl}-N-(2-chlorobenzyl)- |
| | 5-cyclobutyl-1H-pyrazole-1-carboxamide |
| 1150 | 3-{4-[(1-benzothien-2-ylcarbonyl)amino]-2-hydroxyphenyl}-5-cyclobutyl-N-(4- |
| | fluorobenzyl)-1H-pyrazole-1-carboxamide |
| 1151 | 3-{4-[(1-benzothien-2-ylcarbonyl)amino]-2-hydroxyphenyl}-5-cyclobutyl-N-(3,4- |
| | dichlorobenzyl)-1H-pyrazole-1-carboxamide |
| 1152 | 3-{4-[(1-benzothien-2-ylcarbonyl)amino]-2-hydroxyphenyl}-5-cyclobutyl-N-(4- |
| | methoxybenzyl)-1H-pyrazole-1-carboxamide |
| 1153 | 3-{4-[(1-benzothien-2-ylcarbonyl)amino]-2-hydroxyphenyl}-5-cyclobutyl-N-(2- |
| | fluorobenzyl)-1H-pyrazole-1-carboxamide |
| 1154 | 3-{4-[(1-benzothien-2-ylcarbonyl)amino]-2-hydroxyphenyl}-5-cyclobutyl-N-(2- |
| | fluorobenzyl)-1H-pyrazole-1-carboxamide |
| 1155 | 3-{4-[(1-benzothien-2-ylcarbonyl)amino]-2-hydroxyphenyl}-5-cyclobutyl-N-(3- |
| | fluorobenzyl)-1H-pyrazole-1-carboxamide |
| 1156 | 3-{4-[(1-benzothien-2-ylcarbonyl)amino]-2-hydroxyphenyl}-5-cyclobutyl-N- |
| | cyclopropyl-1H-pyrazole-1-carboxamide |
| 1157 | 3-{4-[(1-benzothien-2-ylcarbonyl)amino]-2-hydroxyphenyl}-5-cyclopentyl-N- |
| | isopropyl-1H-pyrazole-1-carboxamide |
| 1158 | 3-{4-[(1-benzothien-2-ylcarbonyl)amino]-2-hydroxyphenyl}-5-cyclopentyl-N- |
| | ethyl-1H-pyrazole-1-carboxamide |
| 1159 | N-allyl-3-{4-[(1-benzothien-2-ylcarbonyl)amino]-2-hydroxyphenyl}-5- |
| | cyclopentyl-1H-pyrazole-1-carboxamide |
| 1160 | 3-{4-[(1-benzothien-2-ylcarbonyl)amino]-2-hydroxyphenyl}-5-cyclopentyl-N- |
| | propyl-1H-pyrazole-1-carboxamide |
| 1161 | 3-{4-[(1-benzothien-2-ylcarbonyl)amino]-2-hydroxyphenyl}-N-benzyl-5- |
| | cyclopentyl-1H-pyrazole-1-carboxamide |
| 1162 | 3-{4-[(1-benzothien-2-ylcarbonyl)amino]-2-hydroxyphenyl}-5-cyclopentyl-N-(2- |
| | phenylethyl)-1H-pyrazole-1-carboxamide |
| 1163 | 3-{4-[(1-benzothien-2-ylcarbonyl)amino]-2-hydroxyphenyl}-5-cyclopentyl-N-(2- |
| | methylbenzyl)-1H-pyrazole-1-carboxamide |
| 1164 | 3-{4-[(1-benzothien-2-ylcarbonyl)amino]-2-hydroxyphenyl}-5-cyclopentyl-N-(3- |
| | methylbenzyl)-1H-pyrazole-1-carboxamide |
| 1165 | 3-{4-[(1-benzothien-2-ylcarbonyl)amino]-2-hydroxyphenyl}-5-cyclopentyl-N-(4- |
| | methylbenzyl)-1H-pyrazole-1-carboxamide |
| 1166 | 3-{4-[(1-benzothien-2-ylcarbonyl)amino]-2-hydroxyphenyl}-N-(2-chlorobenzyl)- |
| | 5-cyclopentyl-1H-pyrazole-1-carboxamide |
| 1167 | 3-{4-[(1-benzothien-2-ylcarbonyl)amino]-2-hydroxyphenyl}-5-cyclopentyl-N-(4- |
| | fluorobenzyl)-1H-pyrazole-1-carboxamide |
| 1168 | 3-{4-[(1-benzothien-2-ylcarbonyl)amino]-2-hydroxyphenyl}-5-cyclopentyl-N- |
| | (3,4-dichlorobenzyl)-1H-pyrazole-1-carboxamide |
| 1169 | 3-{4-[(1-benzothien-2-ylcarbonyl)amino]-2-hydroxyphenyl}-5-cyclopentyl-N-(4- |
| | methoxybenzyl)-1H-pyrazole-1-carboxamide |
| 1170 | 3-{4-[(1-benzothien-2-ylcarbonyl)amino]-2-hydroxyphenyl}-N-(sec-butyl)-5- |
| | cyclopentyl-1H-pyrazole-1-carboxamide |
| 1171 | 3-{4-[(1-benzothien-2-ylcarbonyl)amino]-2-hydroxyphenyl}-5-cyclopentyl-N-(2- |
| | fluorobenzyl)-1H-pyrazole-1-carboxamide |
| 1172 | 3-{4-[(1-benzothien-2-ylcarbonyl)amino]-2-hydroxyphenyl}-5-cyclopentyl-N-(3- |
| | fluorobenzyl)-1H-pyrazole-1-carboxamide |
| 1173 | 3-{4-[(1-benzothien-2-ylcarbonyl)amino]-2-hydroxyphenyl}-5-cyclopentyl-N- |
| | cyclopropyl-1H-pyrazole-1-carboxamide |
| 1174 | 2-chloro-N-(4-{5-cyclopropyl-1-[(isopropylamino)carbonyl]-1H-pyrazol-3-yl}-3- |
| | hydroxyphenyl)nicotinamide |
| 1175 | 2-chloro-N-(4-{5-cyclopropyl-1-[(ethylamino)carbonyl]-1H-pyrazol-3-yl}-3- |
| | hydroxyphenyl)nicotinamide |
| 1176 | N-(4-{1-[(allylamino)carbonyl]-5-cyclopropyl-1H-pyrazol-3-yl}-3- |
| | hydroxyphenyl)-2-chloronicotinamide |
| 1177 | 2-chloro-N-(4-{5-cyclopropyl-1-[(propylamino)carbonyl]-1H-pyrazol-3-yl}-3- |
| | hydroxyphenyl)nicotinamide |
| 1178 | N-(4-{1-[(benzylamino)carbonyl]-5-cyclopropyl-1H-pyrazol-3-yl}-3- |
| | hydroxyphenyl)-2-chloronicotinamide |
| 1179 | 2-chloro-N-[4-(5-cyclopropyl-1-{[(2-phenylethyl)amino]carbonyl}-1H-pyrazol-3- |
| | yl)-3-hydroxyphenyl]nicotinamide |
| 1180 | 2-chloro-N-[4-(5-cyclopropyl-1-{[(2-methylbenzyl)amino]carbonyl}-1H-pyrazol- |
| | 3-yl)-3-hydroxyphenyl]nicotinamide |
| 1181 | 2-chloro-N-[4-(5-cyclopropyl-1-{[(3-methylbenzyl)amino]carbonyl}-1H-pyrazol- |
| | 3-yl)-3-hydroxyphenyl]nicotinamide |
| 1182 | 2-chloro-N-[4-(5-cyclopropyl-1-{[(4-methylbenzyl)amino]carbonyl}-1H-pyrazol- |
| | 3-yl)-3-hydroxyphenyl]nicotinamide |
| 1183 | 2-chloro-N-[4-(1-{[(2-chlorobenzyl)amino]carbonyl}-5-cyclopropyl-1H-pyrazol- |
| | 3-yl)-3-hydroxyphenyl]nicotinamide |
| 1184 | 2-chloro-N-[4-(5-cyclopropyl-1-{[(4-fluorobenzyl)amino]carbonyl}-1H-pyrazol- |
| | 3-yl)-3-hydroxyphenyl]nicotinamide |
| 1185 | 2-chloro-N-[4-(5-cyclopropyl-1-{[(3,4-dichlorobenzyl)amino]carbonyl}-1H- |
| | pyrazol-3-yl)-3-hydroxyphenyl]nicotinamide |
| 1186 | 2-chloro-N-[4-(5-cyclopropyl-1-{[(4-methoxybenzyl)amino]carbonyl}-1H- |
| | pyrazol-3-yl)-3-hydroxyphenyl]nicotinamide |
| 1187 | N-(4-{1-[(sec-butylamino)carbonyl]-5-cyclopropyl-1H-pyrazol-3-yl}-3- |
| | hydroxyphenyl)-2-chloronicotinamide |
| 1188 | 2-chloro-N-[4-(5-cyclopropyl-1-{[(2-fluorobenzyl)amino]carbonyl}-1H-pyrazol- |
| | 3-yl)-3-hydroxyphenyl]nicotinamide |
| 1189 | 2-chloro-N-[4-(5-cyclopropyl-1-{[(3-fluorobenzyl)amino]carbonyl}-1H-pyrazol- |
| | 3-yl)-3-hydroxyphenyl]nicotinamide |
| 1190 | 2-chloro-N-(4-{5-cyclopropyl-1-[(cyclopropylamino)carbonyl]-1H-pyrazol-3-yl}- |
| | 3-hydroxyphenyl)nicotinamide |
| 1191 | 2-chloro-N-(4-{5-cyclobutyl-1-[(isopropylamino)carbonyl]-1H-pyrazol-3-yl}-3- |
| | hydroxyphenyl)nicotinamide |
| 1192 | 2-chloro-N-(4-{5-cyclobutyl-1-[(ethylamino)carbonyl]-1H-pyrazol-3-yl}-3- |
| | hydroxyphenyl)nicotinamide |
| 1193 | N-(4-{1-[(allylamino)carbonyl]-5-cyclobutyl-1H-pyrazol-3-yl}-3-hydroxyphenyl)- |
| | 2-chloronicotinamide |
| 1194 | 2-chloro-N-(4-{5-cyclobutyl-1-[(propylamino)carbonyl]-1H-pyrazol-3-yl}-3- |
| | hydroxyphenyl)nicotinamide |
| 1195 | N-(4-{1-[(benzylamino)carbonyl]-5-cyclobutyl-1H-pyrazol-3-yl}-3- |
| | hydroxyphenyl)-2-chloronicotinamide |
| 1196 | 2-chloro-N-[4-(5-cyclobutyl-1-{[(2-phenylethyl)amino]carbonyl}-1H-pyrazol-3- |
| | yl)-3-hydroxyphenyl]nicotinamide |
| 1197 | 2-chloro-N-[4-(5-cyclobutyl-1-{[(2-methylbenzyl)amino]carbonyl}-1H-pyrazol-3- |
| | yl)-3-hydroxyphenyl]nicotinamide |
| 1198 | 2-chloro-N-[4-(5-cyclobutyl-1-{[(3-methylbenzyl)amino]carbonyl}-1H-pyrazol-3- |
| | yl)-3-hydroxyphenyl]nicotinamide |
| 1199 | 2-chloro-N-[4-(5-cyclobutyl-1-{[(4-methylbenzyl)amino]carbonyl}-1H-pyrazol-3- |
| | yl)-3-hydroxyphenyl]nicotinamide |
| 1200 | 2-chloro-N-[4-(1-{[(2-chlorobenzyl)amino]carbonyl}-5-cyclobutyl-1H-pyrazol-3- |
| | yl)-3-hydroxyphenyl]nicotinamide |
| 1201 | 2-chloro-N-[4-(5-cyclobutyl-1-{[(4-fluorobenzyl)amino]carbonyl}-1H-pyrazol-3- |
| | yl)-3-hydroxyphenyl]nicotinamide |
| 1202 | 2-chloro-N-[4-(5-cyclobutyl-1-{[(3,4-dichlorobenzyl)amino]carbonyl}-1H- |
| | pyrazol-3-yl)-3-hydroxyphenyl]nicotinamide |
| 1203 | 2-chloro-N-[4-(5-cyclobutyl-1-{[(4-methoxybenzyl)amino]carbonyl}-1H-pyrazol- |
| | 3-yl)-3-hydroxyphenyl]nicotinamide |
| 1204 | N-(4-{1-[(sec-butylamino)carbonyl]-5-cyclobutyl-1H-pyrazol-3-yl}-3- |
| | hydroxyphenyl)-2-chloronicotinamide |
| 1205 | 2-chloro-N-[4-(5-cyclobutyl-1-{[(2-fluorobenzyl)amino]carbonyl}-1H-pyrazol-3- |
| | yl)-3-hydroxyphenyl]nicotinamide |
| 1206 | 2-chloro-N-[4-(5-cyclobutyl-1-{[(3-fluorobenzyl)amino]carbonyl}-1H-pyrazol-3- |
| | yl)-3-hydroxyphenyl]nicotinamide |
| 1207 | 2-chloro-N-(4-{5-cyclobutyl-1-[(cyclopropylamino)carbonyl]-1H-pyrazol-3-yl}-3- |
| | hydroxyphenyl)nicotinamide |
| 1208 | 2-chloro-N-(4-{5-cyclopentyl-1-[(isopropylamino)carbonyl]-1H-pyrazol-3-yl}-3- |
| | hydroxyphenyl)nicotinamide |
| 1209 | 2-chloro-N-(4-{5-cyclopentyl-1-[(ethylamino)carbonyl]-1H-pyrazol-3-yl}-3- |
| | hydroxyphenyl)nicotinamide |
| 1210 | N-(4-{1-[(allylamino)carbonyl]-5-cyclopentyl-1H-pyrazol-3-yl}-3- |
| | hydroxyphenyl)-2-chloronicotinamide |
| 1211 | 2-chloro-N-(4-{5-cyclopentyl-1-[(propylamino)carbonyl]-1H-pyrazol-3-yl}-3- |
| | hydroxyphenyl)nicotinamide |
| 1212 | N-(4-{1-[(benzylamino)carbonyl]-5-cyclopentyl-1H-pyrazol-3-yl}-3- |
| | hydroxyphenyl)-2-chloronicotinamide |
| 1213 | 2-chloro-N-[4-(5-cyclopentyl-1-{[(2-phenylethyl)amino]carbonyl}-1H-pyrazol-3- |
| | yl)-3-hydroxyphenyl]nicotinamide |
| 1214 | 2-chloro-N-[4-(5-cyclopentyl-1-{[(2-methylbenzyl)amino]carbonyl}-1H-pyrazol- |
| | 3-yl)-3-hydroxyphenyl]nicotinamide |
| 1215 | 2-chloro-N-[4-(5-cyclopentyl-1-{[(3-methylbenzyl)amino]carbonyl}-1H-pyrazol- |
| | 3-yl)-3-hydroxyphenyl]nicotinamide |
| 1216 | 2-chloro-N-[4-(5-cyclopentyl-1-{[(4-methylbenzyl)amino]carbonyl}-1H-pyrazol- |
| | 3-yl)-3-hydroxyphenyl]nicotinamide |
| 1217 | 2-chloro-N-[4-(1-{[(2-chlorobenzyl)amino]carbonyl}-5-cyclopentyl-1H-pyrazol- |
| | 3-yl)-3-hydroxyphenyl]nicotinamide |
| 1218 | 2-chloro-N-[4-(5-cyclopentyl-1-{[(4-fluorobenzyl)amino]carbonyl}-1H-pyrazol-3- |
| | yl)-3-hydroxyphenyl]nicotinamide |
| 1219 | 2-chloro-N-[4-(5-cyclopentyl-1-{[(3,4-dichlorobenzyl)amino]carbonyl}-1H- |
| | pyrazol-3-yl)-3-hydroxyphenyl]nicotinamide |
| 1220 | 2-chloro-N-[4-(5-cyclopentyl-1-{[(4-methoxybenzyl)amino]carbonyl}-1H- |
| | pyrazol-3-yl)-3-hydroxyphenyl]nicotinamide |
| 1221 | N-(4-{1-[(sec-butylamino)carbonyl]-5-cyclopentyl-1H-pyrazol-3-yl}-3- |
| | hydroxyphenyl)-2-chloronicotinamide |
| 1222 | 2-chloro-N-[4-(5-cyclopentyl-1-{[(2-fluorobenzyl)amino]carbonyl}-1H-pyrazol-3- |
| | yl)-3-hydroxyphenyl]nicotinamide |
| 1223 | 2-chloro-N-[4-(5-cyclopentyl-1-{[(3-fluorobenzyl)amino]carbonyl}-1H-pyrazol-3- |
| | yl)-3-hydroxyphenyl]nicotinamide |
| 1224 | 2-chloro-N-(4-{5-cyclopentyl-1-[(cyclopropylamino)carbonyl]-1H-pyrazol-3-yl}- |
| | 3-hydroxyphenyl)nicotinamide |
| 1225 | 5-cyclopropyl-3-(2-hydroxy-4-{[4-(trifluoromethoxy)benzoyl]amino}phenyl)-N- |
| | isopropyl-1H-pyrazole-1-carboxamide |
| 1226 | 5-cyclopropyl-N-ethyl-3-(2-hydroxy-4-{[4- |
| | (trifluoromethoxy)benzoyl]amino}phenyl)-1H-pyrazole-1-carboxamide |
| 1227 | N-allyl-5-cyclopropyl-3-(2-hydroxy-4-{[4- |
| | (trifluoromethoxy)benzoyl]amino}phenyl)-1H-pyrazole-1-carboxamide |
| 1228 | 5-cyclopropyl-3-(2-hydroxy-4-{[4-(trifluoromethoxy)benzoyl]amino}phenyl)-N- |
| | propyl-1H-pyrazole-1-carboxamide |
| 1229 | N-benzyl-5-cyclopropyl-3-(2-hydroxy-4-{[4- |
| | (trifluoromethoxy)benzoyl]amino}phenyl)-1H-pyrazole-1-carboxamide |
| 1230 | 5-cyclopropyl-3-(2-hydroxy-4-{[4-(trifluoromethoxy)benzoyl]amino}phenyl)-N- |
| | (2-phenylethyl)-1H-pyrazole-1-carboxamide |
| 1231 | 5-cyclopropyl-3-(2-hydroxy-4-{[4-(trifluoromethoxy)benzoyl]amino}phenyl)-N- |
| | (2-methylbenzyl)-1H-pyrazole-1-carboxamide |
| 1232 | 5-cyclopropyl-3-(2-hydroxy-4-{[4-(trifluoromethoxy)benzoyl]amino}phenyl)-N- |
| | (3-methylbenzyl)-1H-pyrazole-1-carboxamide |
| 1233 | 5-cyclopropyl-3-(2-hydroxy-4-{[4-(trifluoromethoxy)benzoyl]amino}phenyl)-N- |
| | (4-methylbenzyl)-1H-pyrazole-1-carboxamide |
| 1234 | N-(2-chlorobenzyl)-5-cyclopropyl-3-(2-hydroxy-4-{[4- |
| | (trifluoromethoxy)benzoyl]amino}phenyl)-1H-pyrazole-1-carboxamide |
| 1235 | 5-cyclopropyl-N-(4-fluorobenzyl)-3-(2-hydroxy-4-{[4- |
| | (trifluoromethoxy)benzoyl]amino}phenyl)-1H-pyrazole-1-carboxamide |
| 1236 | 5-cyclopropyl-N-(3,4-dichlorobenzyl)-3-(2-hydroxy-4-{[4- |
| | (trifluoromethoxy)benzoyl]amino}phenyl)-1H-pyrazole-1-carboxamide |
| 1237 | 5-cyclopropyl-3-(2-hydroxy-4-{[4-(trifluoromethoxy)benzoyl]amino}phenyl)-N- |
| | (4-methoxybenzyl)-1H-pyrazole-1-carboxamide |
| 1238 | N-(sec-butyl)-5-cyclopropyl-3-(2-hydroxy-4-{[4- |
| | (trifluoromethoxy)benzoyl]amino}phenyl)-1H-pyrazole-1-carboxamide |
| 1239 | 5-cyclopropyl-N-(2-fluorobenzyl)-3-(2-hydroxy-4-{[4- |
| | (trifluoromethoxy)benzoyl]amino}phenyl)-1H-pyrazole-1-carboxamide |
| 1240 | 5-cyclopropyl-N-(3-fluorobenzyl)-3-(2-hydroxy-4-{[4- |
| | (trifluoromethoxy)benzoyl]amino}phenyl)-1H-pyrazole-1-carboxamide |
| 1241 | N,5-dicyclopropyl-3-(2-hydroxy-4-{[4-(trifluoromethoxy)benzoyl]amino}phenyl)- |
| | 1H-pyrazole-1-carboxamide |
| 1242 | 5-cyclobutyl-N-ethyl-3-(2-hydroxy-4-{[4- |
| | (trifluoromethoxy)benzoyl]amino}phenyl)-1H-pyrazole-1-carboxamide |
| 1243 | 5-cyclobutyl-N-ethyl-3-(2-hydroxy-4-{[4- |
| | (trifluoromethoxy)benzoyl]amino}phenyl)-1H-pyrazole-1-carboxamide |
| 1244 | N-allyl-5-cyclobutyl-3-(2-hydroxy-4-{[4- |
| | (trifluoromethoxy)benzoyl]amino}phenyl)-1H-pyrazole-1-carboxamide |
| 1245 | 5-cyclobutyl-3-(2-hydroxy-4-{[4-(trifluoromethoxy)benzoyl]amino}phenyl)-N- |
| | propyl-1H-pyrazole-1-carboxamide |
| 1246 | N-benzyl-5-cyclobutyl-3-(2-hydroxy-4-{[4- |
| | (trifluoromethoxy)benzoyl]amino}phenyl)-1H-pyrazole-1-carboxamide |
| 1247 | 5-cyclobutyl-3-(2-hydroxy-4-{[4-(trifluoromethoxy)benzoyl]amino}phenyl)-N-(2- |
| | phenylethyl)-1H-pyrazole-1-carboxamide |
| 1248 | 5-cyclobutyl-3-(2-hydroxy-4-{[4-(trifluoromethoxy)benzoyl]amino}phenyl)-N-(2- |
| | methylbenzyl)-1H-pyrazole-1-carboxamide |
| 1249 | 5-cyclobutyl-3-(2-hydroxy-4-{[4-(trifluoromethoxy)benzoyl]amino}phenyl)-N-(3- |
| | methylbenzyl)-1H-pyrazole-1-carboxamide |
| 1250 | 5-cyclobutyl-3-(2-hydroxy-4-{[4-(trifluoromethoxy)benzoyl]amino}phenyl)-N-(4- |
| | methylbenzyl)-1H-pyrazole-1-carboxamide |
| 1251 | N-(2-chlorobenzyl)-5-cyclobutyl-3-(2-hydroxy-4-{[4- |
| | (trifluoromethoxy)benzoyl]amino}phenyl)-1H-pyrazole-1-carboxamide |
| 1252 | 5-cyclobutyl-N-(4-fluorobenzyl)-3-(2-hydroxy-4-{[4- |
| | (trifluoromethoxy)benzoyl]amino}phenyl)-1H-pyrazole-1-carboxamide |
| 1253 | 5-cyclobutyl-N-(3,4-dichlorobenzyl)-3-(2-hydroxy-4-{[4- |
| | (trifluoromethoxy)benzoyl]amino}phenyl)-1H-pyrazole-1-carboxamide |
| 1254 | 5-cyclobutyl-3-(2-hydroxy-4-{[4-(trifluoromethoxy)benzoyl]amino}phenyl)-N-(4- |
| | methoxybenzyl)-1H-pyrazole-1-carboxamide |
| 1255 | N-(sec-butyl)-5-cyclobutyl-3-(2-hydroxy-4-{[4- |
| | (trifluoromethoxy)benzoyl]amino}phenyl)-1H-pyrazole-1-carboxamide |
| 1256 | 5-cyclobutyl-N-(2-fluorobenzyl)-3-(2-hydroxy-4-{[4- |
| | (trifluoromethoxy)benzoyl]amino}phenyl)-1H-pyrazole-1-carboxamide |
| 1257 | 5-cyclobutyl-N-(3-fluorobenzyl)-3-(2-hydroxy-4-{[4- |
| | (trifluoromethoxy)benzoyl]amino}phenyl)-1H-pyrazole-1-carboxamide |
| 1258 | N-allyl-5-cyclopentyl-3-(2-hydroxy-4-{[4- |
| | (trifluoromethoxy)benzoyl]amino}phenyl)-1H-pyrazole-1-carboxamide |
| 1259 | 5-cyclopentyl-3-(2-hydroxy-4-{[4-(trifluoromethoxy)benzoyl]amino}phenyl)-N- |
| | isopropyl-1H-pyrazole-1-carboxamide |
| 1260 | 5-cyclopentyl-N-ethyl-3-(2-hydroxy-4-{[4- |
| | (trifluoromethoxy)benzoyl]amino}phenyl)-1H-pyrazole-1-carboxamide |
| 1261 | N-allyl-5-cyclopentyl-3-(2-hydroxy-4-{[4- |
| | (trifluoromethoxy)benzoyl]amino}phenyl)-1H-pyrazole-1-carboxamide |
| 1262 | 5-cyclopentyl-3-(2-hydroxy-4-{[4-(trifluoromethoxy)benzoyl]amino}phenyl)-N- |
| | propyl-1H-pyrazole-1-carboxamide |
| 1263 | N-benzyl-5-cyclopentyl-3-(2-hydroxy-4-{[4- |
| | (trifluoromethoxy)benzoyl]amino}phenyl)-1H-pyrazole-1-carboxamide |
| 1264 | 5-cyclopentyl-3-(2-hydroxy-4-{[4-(trifluoromethoxy)benzoyl]amino}phenyl)-N- |
| | (2-phenylethyl)-1H-pyrazole-1-carboxamide |
| 1265 | 5-cyclopentyl-3-(2-hydroxy-4-{[4-(trifluoromethoxy)benzoyl]amino}phenyl)-N- |
| | (2-methylbenzyl)-1H-pyrazole-1-carboxamide |
| 1266 | 5-cyclopentyl-3-(2-hydroxy-4-{[4-(trifluoromethoxy)benzoyl]amino}phenyl)-N- |
| | (3-methylbenzyl)-1H-pyrazole-1-carboxamide |
| 1267 | 5-cyclopentyl-3-(2-hydroxy-4-{[4-(trifluoromethoxy)benzoyl]amino}phenyl)-N- |
| | (4-methylbenzyl)-1H-pyrazole-1-carboxamide |
| 1268 | N-(2-chlorobenzyl)-5-cyclopentyl-3-(2-hydroxy-4-{[4- |
| | (trifluoromethoxy)benzoyl]amino}phenyl)-1H-pyrazole-1-carboxamide |
| 1269 | 5-cyclopentyl-N-(4-fluorobenzyl)-3-(2-hydroxy-4-{[4- |
| | (trifluoromethoxy)benzoyl]amino}phenyl)-1H-pyrazole-1-carboxamide |
| 1270 | 5-cyclopentyl-N-(3,4-dichlorobenzyl)-3-(2-hydroxy-4-{[4- |
| | (trifluoromethoxy)benzoyl]amino}phenyl)-1H-pyrazole-1-carboxamide |
| 1271 | 5-cyclopentyl-3-(2-hydroxy-4-{[4-(trifluoromethoxy)benzoyl]amino}phenyl)-N- |
| | (4-methoxybenzyl)-1H-pyrazole-1-carboxamide |
| 1272 | N-(sec-butyl)-5-cyclopentyl-3-(2-hydroxy-4-{[4- |
| | (trifluoromethoxy)benzoyl]amino}phenyl)-1H-pyrazole-1-carboxamide |
| 1273 | 5-cyclopentyl-N-(2-fluorobenzyl)-3-(2-hydroxy-4-{[4- |
| | (trifluoromethoxy)benzoyl]amino}phenyl)-1H-pyrazole-1-carboxamide |
| 1274 | 5-cyclopentyl-N-(3-fluorobenzyl)-3-(2-hydroxy-4-{[4- |
| | (trifluoromethoxy)benzoyl]amino}phenyl)-1H-pyrazole-1-carboxamide |
| 1275 | 5-cyclopentyl-N-cyclopropyl-3-(2-hydroxy-4-{[4- |
| | (trifluoromethoxy)benzoyl]amino}phenyl)-1H-pyrazole-1-carboxamide |
| 1276 | N-(4-{5-cyclopropyl-1-[(isopropylamino)carbonyl]-1H-pyrazol-3-yl}-3- |
| | hydroxyphenyl)isoxazole-5-carboxamide |
| 1277 | N-(4-{5-cyclopropyl-1-[(ethylamino)carbonyl]-1H-pyrazol-3-yl}-3- |
| | hydroxyphenyl)isoxazole-5-carboxamide |
| 1278 | N-(4-{1-[(allylamino)carbonyl]-5-cyclopropyl-1H-pyrazol-3-yl}-3- |
| | hydroxyphenyl)isoxazole-5-carboxamide |
| 1279 | N-(4-{5-cyclopropyl-1-[(propylamino)carbonyl]-1H-pyrazol-3-yl}-3- |
| | hydroxyphenyl)isoxazole-5-carboxamide |
| 1280 | N-(4-{1-[(benzylamino)carbonyl]-5-cyclopropyl-1H-pyrazol-3-yl}-3- |
| | hydroxyphenyl)isoxazole-5-carboxamide |
| 1281 | N-[4-(5-cyclopropyl-1-{[(2-phenylethyl)amino]carbonyl}-1H-pyrazol-3-yl)-3- |
| | hydroxyphenyl]isoxazole-5-carboxamide |
| 1282 | N-[4-(5-cyclopropyl-1-{[(2-methylbenzyl)amino]carbonyl}-1H-pyrazol-3-yl)-3- |
| | hydroxyphenyl]isoxazole-5-carboxamide |
| 1283 | N-[4-(5-cyclopropyl-1-{[(3-methylbenzyl)amino]carbonyl}-1H-pyrazol-3-yl)-3- |
| | hydroxyphenyl]isoxazole-5-carboxamide |
| 1284 | N-[4-(5-cyclopropyl-1-{[(4-methylbenzyl)amino]carbonyl}-1H-pyrazol-3-yl)-3- |
| | hydroxyphenyl]isoxazole-5-carboxamide |
| 1285 | N-[4-(1-{[(2-chlorobenzyl)amino]carbonyl}-5-cyclopropyl-1H-pyrazol-3-yl)-3- |
| | hydroxyphenyl]isoxazole-5-carboxamide |
| 1286 | N-[4-(5-cyclopropyl-1-{[(4-fluorobenzyl)amino]carbonyl}-1H-pyrazol-3-yl)-3- |
| | hydroxyphenyl]isoxazole-5-carboxamide |
| 1287 | N-[4-(5-cyclopropyl-1-{[(3,4-dichlorobenzyl)amino]carbonyl}-1H-pyrazol-3-yl)- |
| | 3-hydroxyphenyl]isoxazole-5-carboxamide |
| 1288 | N-[4-(5-cyclopropyl-1-{[(4-methoxybenzyl)amino]carbonyl}-1H-pyrazol-3-yl)-3- |
| | hydroxyphenyl]isoxazole-5-carboxamide |
| 1289 | N-(4-{1-[(sec-butylamino)carbonyl]-5-cyclopropyl-1H-pyrazol-3-yl}-3- |
| | hydroxyphenyl)isoxazole-5-carboxamide |
| 1290 | N-[4-(5-cyclopropyl-1-{[(2-fluorobenzyl)amino]carbonyl}-1H-pyrazol-3-yl)-3- |
| | hydroxyphenyl]isoxazole-5-carboxamide |
| 1291 | N-[4-(5-cyclopropyl-1-{[(3-fluorobenzyl)amino]carbonyl}-1H-pyrazol-3-yl)-3- |
| | hydroxyphenyl]isoxazole-5-carboxamide |
| 1292 | N-(4-{5-cyclopropyl-1-[(cyclopropylamino)carbonyl]-1H-pyrazol-3-yl}-3- |
| | hydroxyphenyl)isoxazole-5-carboxamide |
| 1293 | N-(4-{5-cyclobutyl-1-[(isopropylamino)carbonyl]-1H-pyrazol-3-yl}-3- |
| | hydroxyphenyl)isoxazole-5-carboxamide |
| 1294 | N-(4-{5-cyclobutyl-1-[(ethylamino)carbonyl]-1H-pyrazol-3-yl}-3- |
| | hydroxyphenyl)isoxazole-5-carboxamide |
| 1295 | N-(4-{1-[(allylamino)carbonyl]-5-cyclobutyl-1H-pyrazol-3-yl}-3- |
| | hydroxyphenyl)isoxazole-5-carboxamide |
| 1296 | N-(4-{5-cyclobutyl-1-[(propylamino)carbonyl]-1H-pyrazol-3-yl}-3- |
| | hydroxyphenyl)isoxazole-5-carboxamide |
| 1297 | N-(4-{1-[(benzylamino)carbonyl]-5-cyclobutyl-1H-pyrazol-3-yl}-3- |
| | hydroxyphenyl)isoxazole-5-carboxamide |
| 1298 | N-[4-(5-cyclobutyl-1-{[(2-phenylethyl)amino]carbonyl}-1H-pyrazol-3-yl)-3- |
| | hydroxyphenyl]isoxazole-5-carboxamide |
| 1299 | N-[4-(5-cyclobutyl-1-{[(2-methylbenzyl)amino]carbonyl}-1H-pyrazol-3-yl)-3- |
| | hydroxyphenyl]isoxazole-5-carboxamide |
| 1300 | N-[4-(5-cyclobutyl-1-{[(3-methylbenzyl)amino]carbonyl}-1H-pyrazol-3-yl)-3- |
| | hydroxyphenyl]isoxazole-5-carboxamide |
| 1301 | N-[4-(5-cyclobutyl-1-{[(4-methylbenzyl)amino]carbonyl}-1H-pyrazol-3-yl)-3- |
| | hydroxyphenyl]isoxazole-5-carboxamide |
| 1302 | N-[4-(1-{[(2-chlorobenzyl)amino]carbonyl}-5-cyclobutyl-1H-pyrazol-3-yl)-3- |
| | hydroxyphenyl]isoxazole-5-carboxamide |
| 1303 | N-[4-(5-cyclobutyl-1-{[(4-fluorobenzyl)amino]carbonyl}-1H-pyrazol-3-yl)-3- |
| | hydroxyphenyl]isoxazole-5-carboxamide |
| 1304 | N-[4-(5-cyclobutyl-1-{[(3,4-dichlorobenzyl)amino]carbonyl}-1H-pyrazol-3-yl)-3- |
| | hydroxyphenyl]isoxazole-5-carboxamide |
| 1305 | N-[4-(5-cyclobutyl-1-{[(4-methoxybenzyl)amino]carbonyl}-1H-pyrazol-3-yl)-3- |
| | hydroxyphenyl]isoxazole-5-carboxamide |
| 1306 | N-(4-{1-[(sec-butylamino)carbonyl]-5-cyclobutyl-1H-pyrazol-3-yl}-3- |
| | hydroxyphenyl)isoxazole-5-carboxamide |
| 1307 | N-[4-(5-cyclobutyl-1-{[(2-fluorobenzyl)amino]carbonyl}-1H-pyrazol-3-yl)-3- |
| | hydroxyphenyl]isoxazole-5-carboxamide |
| 1308 | N-[4-(5-cyclobutyl-1-{[(3-fluorobenzyl)amino]carbonyl}-1H-pyrazol-3-yl)-3- |
| | hydroxyphenyl]isoxazole-5-carboxamide |
| 1309 | N-(4-{5-cyclobutyl-1-[(cyclopropylamino)carbonyl]-1H-pyrazol-3-yl}-3- |
| | hydroxyphenyl)isoxazole-5-carboxamide |
| 1310 | N-(4-{5-cyclopentyl-1-[(isopropylamino)carbonyl]-1H-pyrazol-3-yl}-3- |
| | hydroxyphenyl)isoxazole-5-carboxamide |
| 1311 | N-(4-{5-cyclopentyl-1-[(ethylamino)carbonyl]-1H-pyrazol-3-yl}-3- |
| | hydroxyphenyl)isoxazole-5-carboxamide |
| 1312 | N-(4-{1-[(allylamino)carbonyl]-5-cyclopentyl-1H-pyrazol-3-yl}-3- |
| | hydroxyphenyl)isoxazole-5-carboxamide |
| 1313 | N-(4-{5-cyclopentyl-1-[(propylamino)carbonyl]-1H-pyrazol-3-yl}-3- |
| | hydroxyphenyl)isoxazole-5-carboxamide |
| 1314 | N-(4-{1-[(benzylamino)carbonyl]-5-cyclopentyl-1H-pyrazol-3-yl}-3- |
| | hydroxyphenyl)isoxazole-5-carboxamide |
| 1315 | N-[4-(5-cyclopentyl-1-{[(2-phenylethyl)amino]carbonyl}-1H-pyrazol-3-yl)-3- |
| | hydroxyphenyl]isoxazole-5-carboxamide |
| 1316 | N-[4-(5-cyclopentyl-1-{[(2-methylbenzyl)amino]carbonyl}-1H-pyrazol-3-yl)-3- |
| | hydroxyphenyl]isoxazole-5-carboxamide |
| 1317 | N-[4-(5-cyclopentyl-1-{[(3-methylbenzyl)amino]carbonyl}-1H-pyrazol-3-yl)-3- |
| | hydroxyphenyl]isoxazole-5-carboxamide |
| 1318 | N-[4-(5-cyclopentyl-1-{[(4-methylbenzyl)amino]carbonyl}-1H-pyrazol-3-yl)-3- |
| | hydroxyphenyl]isoxazole-5-carboxamide |
| 1319 | N-[4-(1-{[(2-chlorobenzyl)amino]carbonyl}-5-cyclopentyl-1H-pyrazol-3-yl)-3- |
| | hydroxyphenyl]isoxazole-5-carboxamide |
| 1320 | N-[4-(5-cyclopentyl-1-{[(4-fluorobenzyl)amino]carbonyl}-1H-pyrazol-3-yl)-3- |
| | hydroxyphenyl]isoxazole-5-carboxamide |
| 1321 | N-[4-(5-cyclopentyl-1-{[(3,4-dichlorobenzyl)amino]carbonyl}-1H-pyrazol-3-yl)- |
| | 3-hydroxyphenyl]isoxazole-5-carboxamide |
| 1322 | N-[4-(5-cyclopentyl-1-{[(4-methoxybenzyl)amino]carbonyl}-1H-pyrazol-3-yl)-3- |
| | hydroxyphenyl]isoxazole-5-carboxamide |
| 1323 | N-(4-{1-[(sec-butylamino)carbonyl]-5-cyclopentyl-1H-pyrazol-3-yl}-3- |
| | hydroxyphenyl)isoxazole-5-carboxamide |
| 1324 | N-[4-(5-cyclopentyl-1-{[(2-fluorobenzyl)amino]carbonyl}-1H-pyrazol-3-yl)-3- |
| | hydroxyphenyl]isoxazole-5-carboxamide |
| 1325 | N-[4-(5-cyclopentyl-1-{[(3-fluorobenzyl)amino]carbonyl}-1H-pyrazol-3-yl)-3- |
| | hydroxyphenyl]isoxazole-5-carboxamide |
| 1326 | N-(4-{5-cyclopentyl-1-[(cyclopropylamino)carbonyl]-1H-pyrazol-3-yl}-3- |
| | hydroxyphenyl)isoxazole-5-carboxamide |
| 1327 | 5-cyclopropyl-3-(2-hydroxy-4-{[(1-methyl-1H-pyrrol-2- |
| | yl)carbonyl]amino}phenyl)-N-isopropyl-1H-pyrazole-1-carboxamide |
| 1328 | 5-cyclopropyl-N-ethyl-3-(2-hydroxy-4-{[(1-methyl-1H-pyrrol-2- |
| | yl)carbonyl]amino}phenyl)-1H-pyrazole-1-carboxamide |
| 1329 | N-allyl-5-cyclopropyl-3-(2-hydroxy-4-{[(1-methyl-1H-pyrrol-2- |
| | yl)carbonyl]amino}phenyl)-1H-pyrazole-1-carboxamide |
| 1330 | 5-cyclopropyl-3-(2-hydroxy-4-{[(1-methyl-1H-pyrrol-2- |
| | yl)carbonyl]amino}phenyl)-N-propyl-1H-pyrazole-1-carboxamide |
| 1331 | N-benzyl-5-cyclopropyl-3-(2-hydroxy-4-{[(1-methyl-1H-pyrrol-2- |
| | yl)carbonyl]amino}phenyl)-1H-pyrazole-1-carboxamide |
| 1332 | 5-cyclopropyl-3-(2-hydroxy-4-{[(1-methyl-1H-pyrrol-2- |
| | yl)carbonyl]amino}phenyl)-N-(2-phenylethyl)-1H-pyrazole-1-carboxamide |
| 1333 | 5-cyclopropyl-3-(2-hydroxy-4-{[(1-methyl-1H-pyrrol-2- |
| | yl)carbonyl]amino}phenyl)-N-(2-methylbenzyl)-1H-pyrazole-1-carboxamide |
| 1334 | 5-cyclopropyl-3-(2-hydroxy-4-{[(1-methyl-1H-pyrrol-2- |
| | yl)carbonyl]amino}phenyl)-N-(3-methylbenzyl)-1H-pyrazole-1-carboxamide |
| 1335 | 5-cyclopropyl-3-(2-hydroxy-4-{[(1-methyl-1H-pyrrol-2- |
| | yl)carbonyl]amino}phenyl)-N-(4-methylbenzyl)-1H-pyrazole-1-carboxamide |
| 1336 | N-(2-chlorobenzyl)-5-cyclopropyl-3-(2-hydroxy-4-{[(1-methyl-1H-pyrrol-2- |
| | yl)carbonyl]amino}phenyl)-1H-pyrazole-1-carboxamide |
| 1337 | 5-cyclopropyl-N-(4-fluorobenzyl)-3-(2-hydroxy-4-{[(1-methyl-1H-pyrrol-2- |
| | yl)carbonyl]amino}phenyl)-1H-pyrazole-1-carboxamide |
| 1338 | 5-cyclopropyl-N-(3,4-dichlorobenzyl)-3-(2-hydroxy-4-{[(1-methyl-1H-pyrrol-2- |
| | yl)carbonyl]amino}phenyl)-1H-pyrazole-1-carboxamide |
| 1339 | 5-cyclopropyl-3-(2-hydroxy-4-{[(1-methyl-1H-pyrrol-2- |
| | yl)carbonyl]amino}phenyl)-N-(4-methoxybenzyl)-1H-pyrazole-1-carboxamide |
| 1340 | N-(sec-butyl)-5-cyclopropyl-3-(2-hydroxy-4-{[(1-methyl-1H-pyrrol-2- |
| | yl)carbonyl]amino}phenyl)-1H-pyrazole-1-carboxamide |
| 1341 | 5-cyclopropyl-N-(2-fluorobenzyl)-3-(2-hydroxy-4-{[(1-methyl-1H-pyrrol-2- |
| | yl)carbonyl]amino}phenyl)-1H-pyrazole-1-carboxamide |
| 1342 | 5-cyclopropyl-N-(3-fluorobenzyl)-3-(2-hydroxy-4-{[(1-methyl-1H-pyrrol-2- |
| | yl)carbonyl]amino}phenyl)-1H-pyrazole-1-carboxamide |
| 1343 | N,5-dicyclopropyl-3-(2-hydroxy-4-{[(1-methyl-1H-pyrrol-2- |
| | yl)carbonyl]amino}phenyl)-1H-pyrazole-1-carboxamide |
| 1344 | 5-cyclobutyl-3-(2-hydroxy-4-{[(1-methyl-1H-pyrrol-2- |
| | yl)carbonyl]amino}phenyl)-N-isopropyl-1H-pyrazole-1-carboxamide |
| 1345 | 5-cyclobutyl-N-ethyl-3-(2-hydroxy-4-{[(1-methyl-1H-pyrrol-2- |
| | yl)carbonyl]amino}phenyl)-1H-pyrazole-1-carboxamide |
| 1346 | N-allyl-5-cyclobutyl-3-(2-hydroxy-4-{[(1-methyl-1H-pyrrol-2- |
| | yl)carbonyl]amino}phenyl)-1H-pyrazole-1-carboxamide |
| 1347 | 5-cyclobutyl-3-(2-hydroxy-4-{[(1-methyl-1H-pyrrol-2- |
| | yl)carbonyl]amino}phenyl)-N-propyl-1H-pyrazole-1-carboxamide |
| 1348 | N-benzyl-5-cyclobutyl-3-(2-hydroxy-4-{[(1-methyl-1H-pyrrol-2- |
| | yl)carbonyl]amino}phenyl)-1H-pyrazole-1-carboxamide |
| 1349 | 5-cyclobutyl-3-(2-hydroxy-4-{[(1-methyl-1H-pyrrol-2- |
| | yl)carbonyl]amino}phenyl)-N-(2-phenylethyl)-1H-pyrazole-1-carboxamide |
| 1350 | 5-cyclobutyl-3-(2-hydroxy-4-{[(1-methyl-1H-pyrrol-2- |
| | yl)carbonyl]amino}phenyl)-N-(2-methylbenzyl)-1H-pyrazole-1-carboxamide |
| 1351 | 5-cyclobutyl-3-(2-hydroxy-4-{[(1-methyl-1H-pyrrol-2- |
| | yl)carbonyl]amino}phenyl)-N-(3-methylbenzyl)-1H-pyrazole-1-carboxamide |
| 1352 | 5-cyclobutyl-3-(2-hydroxy-4-{[(1-methyl-1H-pyrrol-2- |
| | yl)carbonyl]amino}phenyl)-N-(4-methylbenzyl)-1H-pyrazole-1-carboxamide |
| 1353 | N-(2-chlorobenzyl)-5-cyclobutyl-3-(2-hydroxy-4-{[(1-methyl-1H-pyrrol-2- |
| | yl)carbonyl]amino}phenyl)-1H-pyrazole-1-carboxamide |
| 1354 | 5-cyclobutyl-N-(4-fluorobenzyl)-3-(2-hydroxy-4-{[(1-methyl-1H-pyrrol-2- |
| | yl)carbonyl]amino}phenyl)-1H-pyrazole-1-carboxamide |
| 1355 | 5-cyclobutyl-N-(3,4-dichlorobenzyl)-3-(2-hydroxy-4-{[(1-methyl-1H-pyrrol-2- |
| | yl)carbonyl]amino}phenyl)-1H-pyrazole-1-carboxamide |
| 1356 | 5-cyclobutyl-3-(2-hydroxy-4-{[(1-methyl-1H-pyrrol-2- |
| | yl)carbonyl]amino}phenyl)-N-(4-methoxybenzyl)-1H-pyrazole-1-carboxamide |
| 1357 | N-(sec-butyl)-5-cyclobutyl-3-(2-hydroxy-4-{[(1-methyl-1H-pyrrol-2- |
| | yl)carbonyl]amino}phenyl)-1H-pyrazole-1-carboxamide |
| 1358 | 5-cyclobutyl-N-(2-fluorobenzyl)-3-(2-hydroxy-4-{[(1-methyl-1H-pyrrol-2- |
| | yl)carbonyl]amino}phenyl)-1H-pyrazole-1-carboxamide |
| 1359 | 5-cyclobutyl-N-(3-fluorobenzyl)-3-(2-hydroxy-4-{[(1-methyl-1H-pyrrol-2- |
| | yl)carbonyl]amino}phenyl)-1H-pyrazole-1-carboxamide |
| 1360 | 5-cyclobutyl-N-cyclopropyl-3-(2-hydroxy-4-{[(1-methyl-1H-pyrrol-2- |
| | yl)carbonyl]amino}phenyl)-1H-pyrazole-1-carboxamide |
| 1361 | 5-cyclopentyl-3-(2-hydroxy-4-{[(1-methyl-1H-pyrrol-2- |
| | yl)carbonyl]amino}phenyl)-N-isopropyl-1H-pyrazole-1-carboxamide |
| 1362 | 5-cyclopentyl-N-ethyl-3-(2-hydroxy-4-{[(1-methyl-1H-pyrrol-2- |
| | yl)carbonyl]amino}phenyl)-1H-pyrazole-1-carboxamide |
| 1363 | N-allyl-5-cyclopentyl-3-(2-hydroxy-4-{[(1-methyl-1H-pyrrol-2- |
| | yl)carbonyl]amino}phenyl)-1H-pyrazole-1-carboxamide |
| 1364 | 5-cyclopentyl-3-(2-hydroxy-4-{[(1-methyl-1H-pyrrol-2- |
| | yl)carbonyl]amino}phenyl)-N-propyl-1H-pyrazole-1-carboxamide |
| 1365 | N-benzyl-5-cyclopentyl-3-(2-hydroxy-4-{[(1-methyl-1H-pyrrol-2- |
| | yl)carbonyl]amino}phenyl)-1H-pyrazole-1-carboxamide |
| 1366 | 5-cyclopentyl-3-(2-hydroxy-4-{[(1-methyl-1H-pyrrol-2- |
| | yl)carbonyl]amino}phenyl)-N-(2-phenylethyl)-1H-pyrazole-1-carboxamide |
| 1367 | 5-cyclopentyl-3-(2-hydroxy-4-{[(1-methyl-1H-pyrrol-2- |
| | yl)carbonyl]amino}phenyl)-N-(2-methylbenzyl)-1H-pyrazole-1-carboxamide |
| 1368 | 5-cyclopentyl-3-(2-hydroxy-4-{[(1-methyl-1H-pyrrol-2- |
| | yl)carbonyl]amino}phenyl)-N-(3-methylbenzyl)-1H-pyrazole-1-carboxamide |
| 1369 | 5-cyclopentyl-3-(2-hydroxy-4-{[(1-methyl-1H-pyrrol-2- |
| | yl)carbonyl]amino}phenyl)-N-(4-methylbenzyl)-1H-pyrazole-1-carboxamide |
| 1370 | N-(2-chlorobenzyl)-5-cyclopentyl-3-(2-hydroxy-4-{[(1-methyl-1H-pyrrol-2- |
| | yl)carbonyl]amino}phenyl)-1H-pyrazole-1-carboxamide |
| 1371 | 5-cyclopentyl-N-(4-fluorobenzyl)-3-(2-hydroxy-4-{[(1-methyl-1H-pyrrol-2- |
| | yl)carbonyl]amino}phenyl)-1H-pyrazole-1-carboxamide |
| 1372 | 5-cyclopentyl-N-(3,4-dichlorobenzyl)-3-(2-hydroxy-4-{[(1-methyl-1H-pyrrol-2- |
| | yl)carbonyl]amino}phenyl)-1H-pyrazole-1-carboxamide |
| 1373 | 5-cyclopentyl-3-(2-hydroxy-4-{[(1-methyl-1H-pyrrol-2- |
| | yl)carbonyl]amino}phenyl)-N-(4-methoxybenzyl)-1H-pyrazole-1-carboxamide |
| 1374 | N-(sec-butyl)-5-cyclopentyl-3-(2-hydroxy-4-{[(1-methyl-1H-pyrrol-2- |
| | yl)carbonyl]amino}phenyl)-1H-pyrazole-1-carboxamide |
| 1375 | 5-cyclopentyl-N-(2-fluorobenzyl)-3-(2-hydroxy-4-{[(1-methyl-1H-pyrrol-2- |
| | yl)carbonyl]amino}phenyl)-1H-pyrazole-1-carboxamide |
| 1376 | 5-cyclopentyl-N-(3-fluorobenzyl)-3-(2-hydroxy-4-{[(1-methyl-1H-pyrrol-2- |
| | yl)carbonyl]amino}phenyl)-1H-pyrazole-1-carboxamide |
| 1377 | 5-cyclopentyl-N-cyclopropyl-3-(2-hydroxy-4-{[(1-methyl-1H-pyrrol-2- |
| | yl)carbonyl]amino}phenyl)-1H-pyrazole-1-carboxamide |
| 1378 | 3-{5-[(2-chlorobenzoyl)amino]-2-hydroxyphenyl}-5-cyclopropyl-N-isopropyl-1H- |
| | pyrazole-1-carboxamide |
| 1379 | 3-{5-[(2-chlorobenzoyl)amino]-2-hydroxyphenyl}-5-cyclopropyl-N-ethyl-1H- |
| | pyrazole-1-carboxamide |
| 1380 | N-allyl-3-{5-[(2-chlorobenzoyl)amino]-2-hydroxyphenyl}-5-cyclopropyl-1H- |
| | pyrazole-1-carboxamide |
| 1381 | 3-{5-[(2-chlorobenzoyl)amino]-2-hydroxyphenyl}-5-cyclopropyl-N-propyl-1H- |
| | pyrazole-1-carboxamide |
| 1382 | N-benzyl-3-{5-[(2-chlorobenzoyl)amino]-2-hydroxyphenyl}-5-cyclopropyl-1H- |
| | pyrazole-1-carboxamide |
| 1383 | 3-{5-[(2-chlorobenzoyl)amino]-2-hydroxyphenyl}-5-cyclopropyl-N-(2- |
| | phenylethyl)-1H-pyrazole-1-carboxamide |
| 1384 | 3-{5-[(2-chlorobenzoyl)amino]-2-hydroxyphenyl}-5-cyclopropyl-N-(2- |
| | methylbenzyl)-1H-pyrazole-1-carboxamide |
| 1385 | 3-{5-[(2-chlorobenzoyl)amino]-2-hydroxyphenyl}-5-cyclopropyl-N-(3- |
| | methylbenzyl)-1H-pyrazole-1-carboxamide |
| 1386 | 3-{5-[(2-chlorobenzoyl)amino]-2-hydroxyphenyl}-5-cyclopropyl-N-(4- |
| | methylbenzyl)-1H-pyrazole-1-carboxamide |
| 1387 | 3-{5-[(2-chlorobenzoyl)amino]-2-hydroxyphenyl}-N-(2-chlorobenzyl)-5- |
| | cyclopropyl-1H-pyrazole-1-carboxamide |
| 1388 | 3-{5-[(2-chlorobenzoyl)amino]-2-hydroxyphenyl}-5-cyclopropyl-N-(4- |
| | fluorobenzyl)-1H-pyrazole-1-carboxamide |
| 1389 | 3-{5-[(2-chlorobenzoyl)amino]-2-hydroxyphenyl}-5-cyclopropyl-N-(3,4- |
| | dichlorobenzyl)-1H-pyrazole-1-carboxamide |
| 1390 | 3-{5-[(2-chlorobenzoyl)amino]-2-hydroxyphenyl}-5-cyclopropyl-N-(4- |
| | methoxybenzyl)-1H-pyrazole-1-carboxamide |
| 1391 | N-(sec-butyl)-3-{5-[(2-chlorobenzoyl)amino]-2-hydroxyphenyl}-5-cyclopropyl- |
| | 1H-pyrazole-1-carboxamide |
| 1392 | 3-{5-[(2-chlorobenzoyl)amino]-2-hydroxyphenyl}-5-cyclopropyl-N-(2- |
| | fluorobenzyl)-1H-pyrazole-1-carboxamide |
| 1393 | 3-{5-[(2-chlorobenzoyl)amino]-2-hydroxyphenyl}-5-cyclopropyl-N-(3- |
| | fluorobenzyl)-1H-pyrazole-1-carboxamide |
| 1394 | 3-{5-[(2-chlorobenzoyl)amino]-2-hydroxyphenyl}-N,5-dicyclopropyl-1H- |
| | pyrazole-1-carboxamide |
| 1395 | 3-{5-[(2-chlorobenzoyl)amino]-2-hydroxyphenyl}-5-cyclobutyl-N-isopropyl-1H- |
| | pyrazole-1-carboxamide |
| 1396 | 3-{5-[(2-chlorobenzoyl)amino]-2-hydroxyphenyl}-5-cyclobutyl-N-ethyl-1H- |
| | pyrazole-1-carboxamide |
| 1397 | N-allyl-3-{5-[(2-chlorobenzoyl)amino]-2-hydroxyphenyl}-5-cyclobutyl-1H- |
| | pyrazole-1-carboxamide |
| 1398 | 3-{5-[(2-chlorobenzoyl)amino]-2-hydroxyphenyl}-5-cyclobutyl-N-propyl-1H- |
| | pyrazole-1-carboxamide |
| 1399 | N-benzyl-3-{5-[(2-chlorobenzoyl)amino]-2-hydroxyphenyl}-5-cyclobutyl-1H- |
| | pyrazole-1-carboxamide |
| 1400 | 3-{5-[(2-chlorobenzoyl)amino]-2-hydroxyphenyl}-5-cyclobutyl-N-(2- |
| | phenylethyl)-1H-pyrazole-1-carboxamide |
| 1401 | 3-{5-[(2-chlorobenzoyl)amino]-2-hydroxyphenyl}-5-cyclobutyl-N-(2- |
| | methylbenzyl)-1H-pyrazole-1-carboxamide |
| 1402 | 3-{5-[(2-chlorobenzoyl)amino]-2-hydroxyphenyl}-5-cyclobutyl-N-(3- |
| | methylbenzyl)-1H-pyrazole-1-carboxamide |
| 1403 | 3-{5-[(2-chlorobenzoyl)amino]-2-hydroxyphenyl}-5-cyclobutyl-N-(4- |
| | methylbenzyl)-1H-pyrazole-1-carboxamide |
| 1404 | 3-{5-[(2-chlorobenzoyl)amino]-2-hydroxyphenyl}-N-(2-chlorobenzyl)-5- |
| | cyclobutyl-1H-pyrazole-1-carboxamide |
| 1405 | 3-{5-[(2-chlorobenzoyl)amino]-2-hydroxyphenyl}-5-cyclobutyl-N-(4- |
| | fluorobenzyl)-1H-pyrazole-1-carboxamide |
| 1406 | 3-{5-[(2-chlorobenzoyl)amino]-2-hydroxyphenyl}-5-cyclobutyl-N-(3,4- |
| | dichlorobenzyl)-1H-pyrazole-1-carboxamide |
| 1407 | 3-{5-[(2-chlorobenzoyl)amino]-2-hydroxyphenyl}-5-cyclobutyl-N-(4- |
| | methoxybenzyl)-1H-pyrazole-1-carboxamide |
| 1408 | N-(sec-butyl)-3-{5-[(2-chlorobenzoyl)amino]-2-hydroxyphenyl}-5-cyclobutyl-1H- |
| | pyrazole-1-carboxamide |
| 1409 | 3-{5-[(2-chlorobenzoyl)amino]-2-hydroxyphenyl}-5-cyclobutyl-N-(2- |
| | fluorobenzyl)-1H-pyrazole-1-carboxamide |
| 1410 | 3-{5-[(2-chlorobenzoyl)amino]-2-hydroxyphenyl}-5-cyclobutyl-N-(3- |
| | fluorobenzyl)-1H-pyrazole-1-carboxamide |
| 1411 | 3-{5-[(2-chlorobenzoyl)amino]-2-hydroxyphenyl}-5-cyclobutyl-N-cyclopropyl- |
| | 1H-pyrazole-1-carboxamide |
| 1412 | 3-{5-[(2-chlorobenzoyl)amino]-2-hydroxyphenyl}-5-cyclopentyl-N-isopropyl-1H- |
| | pyrazole-1-carboxamide |
| 1413 | 3-{5-[(2-chlorobenzoyl)amino]-2-hydroxyphenyl}-5-cyclopentyl-N-ethyl-1H- |
| | pyrazole-1-carboxamide |
| 1414 | N-allyl-3-{5-[(2-chlorobenzoyl)amino]-2-hydroxyphenyl}-5-cyclopentyl-1H- |
| | pyrazole-1-carboxamide |
| 1415 | 3-{5-[(2-chlorobenzoyl)amino]-2-hydroxyphenyl}-5-cyclopentyl-N-propyl-1H- |
| | pyrazole-1-carboxamide |
| 1416 | N-benzyl-3-{5-[(2-chlorobenzoyl)amino]-2-hydroxyphenyl}-5-cyclopentyl-1H- |
| | pyrazole-1-carboxamide |
| 1417 | 3-{5-[(2-chlorobenzoyl)amino]-2-hydroxyphenyl}-5-cyclopentyl-N-(2- |
| | phenylethyl)-1H-pyrazole-1-carboxamide |
| 1418 | 3-{5-[(2-chlorobenzoyl)amino]-2-hydroxyphenyl}-5-cyclopentyl-N-(2- |
| | methylbenzyl)-1H-pyrazole-1-carboxamide |
| 1419 | 3-{5-[(2-chlorobenzoyl)amino]-2-hydroxyphenyl}-5-cyclopentyl-N-(3- |
| | methylbenzyl)-1H-pyrazole-1-carboxamide |
| 1420 | 3-{5-[(2-chlorobenzoyl)amino]-2-hydroxyphenyl}-5-cyclopentyl-N-(4- |
| | methylbenzyl)-1H-pyrazole-1-carboxamide |
| 1421 | 3-{5-[(2-chlorobenzoyl)amino]-2-hydroxyphenyl}-N-(2-chlorobenzyl)-5- |
| | cyclopentyl-1H-pyrazole-1-carboxamide |
| 1422 | 3-{5-[(2-chlorobenzoyl)amino]-2-hydroxyphenyl}-5-cyclopentyl-N-(4- |
| | fluorobenzyl)-1H-pyrazole-1-carboxamide |
| 1423 | 3-{5-[(2-chlorobenzoyl)amino]-2-hydroxyphenyl}-5-cyclopentyl-N-(3,4- |
| | dichlorobenzyl)-1H-pyrazole-1-carboxamide |
| 1424 | 3-{5-[(2-chlorobenzoyl)amino]-2-hydroxyphenyl}-5-cyclopentyl-N-(4- |
| | methoxybenzyl)-1H-pyrazole-1-carboxamide |
| 1425 | N-(sec-butyl)-3-{5-[(2-chlorobenzoyl)amino]-2-hydroxyphenyl}-5-cyclopentyl- |
| | 1H-pyrazole-1-carboxamide |
| 1426 | 3-{5-[(2-chlorobenzoyl)amino]-2-hydroxyphenyl}-5-cyclopentyl-N-(2- |
| | fluorobenzyl)-1H-pyrazole-1-carboxamide |
| 1427 | 3-{5-[(2-chlorobenzoyl)amino]-2-hydroxyphenyl}-5-cyclopentyl-N-(3- |
| | fluorobenzyl)-1H-pyrazole-1-carboxamide |
| 1428 | 3-{5-[(2-chlorobenzoyl)amino]-2-hydroxyphenyl}-5-cyclopentyl-N-cyclopropyl- |
| | 1H-pyrazole-1-carboxamide |
| 1429 | 5-cyclopropyl-3-{5-[(2,6-dichlorobenzoyl)amino]-2-hydroxyphenyl}-N-isopropyl- |
| | 1H-pyrazole-1-carboxamide |
| 1430 | 5-cyclopropyl-3-{5-[(2,6-dichlorobenzoyl)amino]-2-hydroxyphenyl}-N-ethyl-1H- |
| | pyrazole-1-carboxamide |
| 1431 | N-allyl-5-cyclopropyl-3-{5-[(2,6-dichlorobenzoyl)amino]-2-hydroxyphenyl}-1H- |
| | pyrazole-1-carboxamide |
| 1432 | 5-cyclopropyl-3-{5-[(2,6-dichlorobenzoyl)amino]-2-hydroxyphenyl}-N-propyl- |
| | 1H-pyrazole-1-carboxamide |
| 1433 | N-benzyl-5-cyclopropyl-3-{5-[(2,6-dichlorobenzoyl)amino]-2-hydroxyphenyl}- |
| | 1H-pyrazole-1-carboxamide |
| 1434 | 5-cyclopropyl-3-{5-[(2,6-dichlorobenzoyl)amino]-2-hydroxyphenyl}-N-(2- |
| | phenylethyl)-1H-pyrazole-1-carboxamide |
| 1435 | 5-cyclopropyl-3-{5-[(2,6-dichlorobenzoyl)amino]-2-hydroxyphenyl}-N-(2- |
| | methylbenzyl)-1H-pyrazole-1-carboxamide |
| 1436 | 5-cyclopropyl-3-{5-[(2,6-dichlorobenzoyl)amino]-2-hydroxyphenyl}-N-(3- |
| | methylbenzyl)-1H-pyrazole-1-carboxamide |
| 1437 | 5-cyclopropyl-3-{5-[(2,6-dichlorobenzoyl)amino]-2-hydroxyphenyl}-N-(4- |
| | methylbenzyl)-1H-pyrazole-1-carboxamide |
| 1438 | N-(2-chlorobenzyl)-5-cyclopropyl-3-{5-[(2,6-dichlorobenzoyl)amino]-2- |
| | hydroxyphenyl}-1H-pyrazole-1-carboxamide |
| 1439 | 5-cyclopropyl-3-{5-[(2,6-dichlorobenzoyl)amino]-2-hydroxyphenyl}-N-(4- |
| | fluorobenzyl)-1H-pyrazole-1-carboxamide |
| 1440 | 5-cyclopropyl-3-{5-[(2,6-dichlorobenzoyl)amino]-2-hydroxyphenyl}-N-(3,4- |
| | dichlorobenzyl)-1H-pyrazole-1-carboxamide |
| 1441 | 5-cyclopropyl-3-{5-[(2,6-dichlorobenzoyl)amino]-2-hydroxyphenyl}-N-(4- |
| | methoxybenzyl)-1H-pyrazole-1-carboxamide |
| 1442 | N-(sec-butyl)-5-cyclopropyl-3-{5-[(2,6-dichlorobenzoyl)amino]-2- |
| | hydroxyphenyl}-1H-pyrazole-1-carboxamide |
| 1443 | 5-cyclopropyl-3-{5-[(2,6-dichlorobenzoyl)amino]-2-hydroxyphenyl}-N-(2- |
| | fluorobenzyl)-1H-pyrazole-1-carboxamide |
| 1444 | 5-cyclopropyl-3-{5-[(2,6-dichlorobenzoyl)amino]-2-hydroxyphenyl}-N-(3- |
| | fluorobenzyl)-1H-pyrazole-1-carboxamide |
| 1445 | N,5-dicyclopropyl-3-{5-[(2,6-dichlorobenzoyl)amino]-2-hydroxyphenyl}-1H- |
| | pyrazole-1-carboxamide |
| 1446 | 5-cyclobutyl-3-{5-[(2,6-dichlorobenzoyl)amino]-2-hydroxyphenyl}-N-isopropyl- |
| | 1H-pyrazole-1-carboxamide |
| 1447 | 5-cyclobutyl-3-{5-[(2,6-dichlorobenzoyl)amino]-2-hydroxyphenyl}-N-ethyl-1H- |
| | pyrazole-1-carboxamide |
| 1448 | N-allyl-5-cyclobutyl-3-{5-[(2,6-dichlorobenzoyl)amino]-2-hydroxyphenyl}-1H- |
| | pyrazole-1-carboxamide |
| 1449 | 5-cyclobutyl-3-{5-[(2,6-dichlorobenzoyl)amino]-2-hydroxyphenyl}-N-propyl-1H- |
| | pyrazole-1-carboxamide |
| 1450 | N-benzyl-5-cyclobutyl-3-{5-[(2,6-dichlorobenzoyl)amino]-2-hydroxyphenyl}-1H- |
| | pyrazole-1-carboxamide |
| 1451 | 5-cyclobutyl-3-{5-[(2,6-dichlorobenzoyl)amino]-2-hydroxyphenyl}-N-(2- |
| | phenylethyl)-1H-pyrazole-1-carboxamide |
| 1452 | 5-cyclobutyl-3-{5-[(2,6-dichlorobenzoyl)amino]-2-hydroxyphenyl}-N-(2- |
| | methylbenzyl)-1H-pyrazole-1-carboxamide |
| 1453 | 5-cyclobutyl-3-{5-[(2,6-dichlorobenzoyl)amino]-2-hydroxyphenyl}-N-(3- |
| | methylbenzyl)-1H-pyrazole-1-carboxamide |
| 1454 | 5-cyclobutyl-3-{5-[(2,6-dichlorobenzoyl)amino]-2-hydroxyphenyl}-N-(4- |
| | methylbenzyl)-1H-pyrazole-1-carboxamide |
| 1455 | N-(2-chlorobenzyl)-5-cyclobutyl-3-{5-[(2,6-dichlorobenzoyl)amino]-2- |
| | hydroxyphenyl}-1H-pyrazole-1-carboxamide |
| 1456 | 5-cyclobutyl-3-{5-[(2,6-dichlorobenzoyl)amino]-2-hydroxyphenyl}-N-(4- |
| | fluorobenzyl)-1H-pyrazole-1-carboxamide |
| 1457 | 5-cyclobutyl-3-{5-[(2,6-dichlorobenzoyl)amino]-2-hydroxyphenyl}-N-(3,4- |
| | dichlorobenzyl)-1H-pyrazole-1-carboxamide |
| 1458 | 5-cyclobutyl-3-{5-[(2,6-dichlorobenzoyl)amino]-2-hydroxyphenyl}-N-(4- |
| | methoxybenzyl)-1H-pyrazole-1-carboxamide |
| 1459 | N-(sec-butyl)-5-cyclobutyl-3-{5-[(2,6-dichlorobenzoyl)amino]-2-hydroxyphenyl}- |
| | 1H-pyrazole-1-carboxamide |
| 1460 | 5-cyclobutyl-3-{5-[(2,6-dichlorobenzoyl)amino]-2-hydroxyphenyl}-N-(2- |
| | fluorobenzyl)-1H-pyrazole-1-carboxamide |
| 1461 | 5-cyclobutyl-3-{5-[(2,6-dichlorobenzoyl)amino]-2-hydroxyphenyl}-N-(3- |
| | fluorobenzyl)-1H-pyrazole-1-carboxamide |
| 1462 | 5-cyclobutyl-N-cyclopropyl-3-{5-[(2,6-dichlorobenzoyl)amino]-2- |
| | hydroxyphenyl}-1H-pyrazole-1-carboxamide |
| 1463 | 5-cyclopentyl-3-{5-[(2,6-dichlorobenzoyl)amino]-2-hydroxyphenyl}-N-isopropyl- |
| | 1H-pyrazole-1-carboxamide |
| 1464 | 5-cyclopentyl-3-{5-[(2,6-dichlorobenzoyl)amino]-2-hydroxyphenyl}-N-ethyl-1H- |
| | pyrazole-1-carboxamide |
| 1465 | N-allyl-5-cyclopentyl-3-{5-[(2,6-dichlorobenzoyl)amino]-2-hydroxyphenyl}-1H- |
| | pyrazole-1-carboxamide |
| 1466 | 5-cyclopentyl-3-{5-[(2,6-dichlorobenzoyl)amino]-2-hydroxyphenyl}-N-propyl- |
| | 1H-pyrazole-1-carboxamide |
| 1467 | N-benzyl-5-cyclopentyl-3-{5-[(2,6-dichlorobenzoyl)amino]-2-hydroxyphenyl}- |
| | 1H-pyrazole-1-carboxamide |
| 1468 | 5-cyclopentyl-3-{5-[(2,6-dichlorobenzoyl)amino]-2-hydroxyphenyl}-N-(2- |
| | phenylethyl)-1H-pyrazole-1-carboxamide |
| 1469 | 5-cyclopentyl-3-{5-[(2,6-dichlorobenzoyl)amino]-2-hydroxyphenyl}-N-(2- |
| | methylbenzyl)-1H-pyrazole-1-carboxamide |
| 1470 | 5-cyclopentyl-3-{5-[(2,6-dichlorobenzoyl)amino]-2-hydroxyphenyl}-N-(3- |
| | methylbenzyl)-1H-pyrazole-1-carboxamide |
| 1471 | 5-cyclopentyl-3-{5-[(2,6-dichlorobenzoyl)amino]-2-hydroxyphenyl}-N-(4- |
| | methylbenzyl)-1H-pyrazole-1-carboxamide |
| 1472 | N-(2-chlorobenzyl)-5-cyclopentyl-3-{5-[(2,6-dichlorobenzoyl)amino]-2- |
| | hydroxyphenyl}-1H-pyrazole-1-carboxamide |
| 1473 | 5-cyclopentyl-3-{5-[(2,6-dichlorobenzoyl)amino]-2-hydroxyphenyl}-N-(4- |
| | fluorobenzyl)-1H-pyrazole-1-carboxamide |
| 1474 | 5-cyclopentyl-3-{5-[(2,6-dichlorobenzoyl)amino]-2-hydroxyphenyl}-N-(3,4- |
| | dichlorobenzyl)-1H-pyrazole-1-carboxamide |
| 1475 | 5-cyclopentyl-3-{5-[(2,6-dichlorobenzoyl)amino]-2-hydroxyphenyl}-N-(4- |
| | methoxybenzyl)-1H-pyrazole-1-carboxamide |
| 1476 | N-(sec-butyl)-5-cyclopentyl-3-{5-[(2,6-dichlorobenzoyl)amino]-2- |
| | hydroxyphenyl}-1H-pyrazole-1-carboxamide |
| 1477 | 5-cyclopentyl-3-{5-[(2,6-dichlorobenzoyl)amino]-2-hydroxyphenyl}-N-(2- |
| | fluorobenzyl)-1H-pyrazole-1-carboxamide |
| 1478 | 5-cyclopentyl-3-{5-[(2,6-dichlorobenzoyl)amino]-2-hydroxyphenyl}-N-(3- |
| | fluorobenzyl)-1H-pyrazole-1-carboxamide |
| 1479 | 5-cyclopentyl-N-cyclopropyl-3-{5-[(2,6-dichlorobenzoyl)amino]-2- |
| | hydroxyphenyl}-1H-pyrazole-1-carboxamide |
| 1480 | 5-cyclopropyl-3-{2-hydroxy-5-[(2-methoxybenzoyl)amino]phenyl}-N-isopropyl- |
| | 1H-pyrazole-1-carboxamide |
| 1481 | 5-cyclopropyl-N-ethyl-3-{2-hydroxy-5-[(2-methoxybenzoyl)amino]phenyl}-1H- |
| | pyrazole-1-carboxamide |
| 1482 | N-allyl-5-cyclopropyl-3-{2-hydroxy-5-[(2-methoxybenzoyl)amino]phenyl}-1H- |
| | pyrazole-1-carboxamide |
| 1483 | 5-cyclopropyl-3-{2-hydroxy-5-[(2-methoxybenzoyl)amino]phenyl}-N-propyl-1H- |
| | pyrazole-1-carboxamide |
| 1484 | N-benzyl-5-cyclopropyl-3-{2-hydroxy-5-[(2-methoxybenzoyl)amino]phenyl}-1H- |
| | pyrazole-1-carboxamide |
| 1485 | 5-cyclopropyl-3-{2-hydroxy-5-[(2-methoxybenzoyl)amino]phenyl}-N-(2- |
| | phenylethyl)-1H-pyrazole-1-carboxamide |
| 1486 | 5-cyclopropyl-3-{2-hydroxy-5-[(2-methoxybenzoyl)amino]phenyl}-N-(2- |
| | methylbenzyl)-1H-pyrazole-1-carboxamide |
| 1487 | 5-cyclopropyl-3-{2-hydroxy-5-[(2-methoxybenzoyl)amino]phenyl}-N-(3- |
| | methylbenzyl)-1H-pyrazole-1-carboxamide |
| 1488 | 5-cyclopropyl-3-{2-hydroxy-5-[(2-methoxybenzoyl)amino]phenyl}-N-(4- |
| | methylbenzyl)-1H-pyrazole-1-carboxamide |
| 1489 | N-(2-chlorobenzyl)-5-cyclopropyl-3-{2-hydroxy-5-[(2- |
| | methoxybenzoyl)amino]phenyl}-1H-pyrazole-1-carboxamide |
| 1490 | 5-cyclopropyl-N-(4-fluorobenzyl)-3-{2-hydroxy-5-[(2- |
| | methoxybenzoyl)amino]phenyl}-1H-pyrazole-1-carboxamide |
| 1491 | 5-cyclopropyl-N-(3,4-dichlorobenzyl)-3-{2-hydroxy-5-[(2- |
| | methoxybenzoyl)amino]phenyl}-1H-pyrazole-1-carboxamide |
| 1492 | 5-cyclopropyl-3-{2-hydroxy-5-[(2-methoxybenzoyl)amino]phenyl}-N-(4- |
| | methoxybenzyl)-1H-pyrazole-1-carboxamide |
| 1493 | N-(sec-butyl)-5-cyclopropyl-3-{2-hydroxy-5-[(2-methoxybenzoyl)amino]phenyl}- |
| | 1H-pyrazole-1-carboxamide |
| 1494 | 5-cyclopropyl-N-(2-fluorobenzyl)-3-{2-hydroxy-5-[(2- |
| | methoxybenzoyl)amino]phenyl}-1H-pyrazole-1-carboxamide |
| 1495 | 5-cyclopropyl-N-(3-fluorobenzyl)-3-{2-hydroxy-5-[(2- |
| | methoxybenzoyl)amino]phenyl}-1H-pyrazole-1-carboxamide |
| 1496 | N,5-dicyclopropyl-3-{2-hydroxy-5-[(2-methoxybenzoyl)amino]phenyl}-1H- |
| | pyrazole-1-carboxamide |
| 1497 | 5-cyclobutyl-3-{2-hydroxy-5-[(2-methoxybenzoyl)amino]phenyl}-N-isopropyl- |
| | 1H-pyrazole-1-carboxamide |
| 1498 | 5-cyclobutyl-N-ethyl-3-{2-hydroxy-5-[(2-methoxybenzoyl)amino]phenyl}-1H- |
| | pyrazole-1-carboxamide |
| 1499 | N-allyl-5-cyclobutyl-3-{2-hydroxy-5-[(2-methoxybenzoyl)amino]phenyl}-1H- |
| | pyrazole-1-carboxamide |
| 1500 | 5-cyclobutyl-3-{2-hydroxy-5-[(2-methoxybenzoyl)amino]phenyl}-N-propyl-1H- |
| | pyrazole-1-carboxamide |
| 1501 | N-benzyl-5-cyclobutyl-3-{2-hydroxy-5-[(2-methoxybenzoyl)amino]phenyl}-1H- |
| | pyrazole-1-carboxamide |
| 1502 | 5-cyclobutyl-3-{2-hydroxy-5-[(2-methoxybenzoyl)amino]phenyl}-N-(2- |
| | phenylethyl)-1H-pyrazole-1-carboxamide |
| 1503 | 5-cyclobutyl-3-{2-hydroxy-5-[(2-methoxybenzoyl)amino]phenyl}-N-(2- |
| | methylbenzyl)-1H-pyrazole-1-carboxamide |
| 1504 | 5-cyclobutyl-3-{2-hydroxy-5-[(2-methoxybenzoyl)amino]phenyl}-N-(3- |
| | methylbenzyl)-1H-pyrazole-1-carboxamide |
| 1505 | 5-cyclobutyl-3-{2-hydroxy-5-[(2-methoxybenzoyl)amino]phenyl}-N-(4- |
| | methylbenzyl)-1H-pyrazole-1-carboxamide |
| 1506 | N-(2-chlorobenzyl)-5-cyclobutyl-3-{2-hydroxy-5-[(2- |
| | methoxybenzoyl)amino]phenyl}-1H-pyrazole-1-carboxamide |
| 1507 | 5-cyclobutyl-N-(4-fluorobenzyl)-3-{2-hydroxy-5-[(2- |
| | methoxybenzoyl)amino]phenyl}-1H-pyrazole-1-carboxamide |
| 1508 | 5-cyclobutyl-N-(3,4-dichlorobenzyl)-3-{2-hydroxy-5-[(2- |
| | methoxybenzoyl)amino]phenyl}-1H-pyrazole-1-carboxamide |
| 1509 | 5-cyclobutyl-3-{2-hydroxy-5-[(2-methoxybenzoyl)amino]phenyl}-N-(4- |
| | methoxybenzyl)-1H-pyrazole-1-carboxamide |
| 1510 | N-(sec-butyl)-5-cyclobutyl-3-{2-hydroxy-5-[(2-methoxybenzoyl)amino]phenyl}- |
| | 1H-pyrazole-1-carboxamide |
| 1511 | 5-cyclobutyl-N-(2-fluorobenzyl)-3-{2-hydroxy-5-[(2- |
| | methoxybenzoyl)amino]phenyl}-1H-pyrazole-1-carboxamide |
| 1512 | 5-cyclobutyl-N-(3-fluorobenzyl)-3-{2-hydroxy-5-[(2- |
| | methoxybenzoyl)amino]phenyl}-1H-pyrazole-1-carboxamide |
| 1513 | 5-cyclobutyl-N-cyclopropyl-3-{2-hydroxy-5-[(2-methoxybenzoyl)amino]phenyl}- |
| | 1H-pyrazole-1-carboxamide |
| 1514 | 5-cyclopentyl-3-{2-hydroxy-5-[(2-methoxybenzoyl)amino]phenyl}-N-isopropyl- |
| | 1H-pyrazole-1-carboxamide |
| 1515 | 5-cyclopentyl-N-ethyl-3-{2-hydroxy-5-[(2-methoxybenzoyl)amino]phenyl}-1H- |
| | pyrazole-1-carboxamide |
| 1516 | N-allyl-5-cyclopentyl-3-{2-hydroxy-5-[(2-methoxybenzoyl)amino]phenyl}-1H- |
| | pyrazole-1-carboxamide |
| 1517 | 5-cyclopentyl-3-{2-hydroxy-5-[(2-methoxybenzoyl)amino]phenyl}-N-propyl-1H- |
| | pyrazole-1-carboxamide |
| 1518 | N-benzyl-5-cyclopentyl-3-{2-hydroxy-5-[(2-methoxybenzoyl)amino]phenyl}-1H- |
| | pyrazole-1-carboxamide |
| 1519 | 5-cyclopentyl-3-{2-hydroxy-5-[(2-methoxybenzoyl)amino]phenyl}-N-(2- |
| | phenylethyl)-1H-pyrazole-1-carboxamide |
| 1520 | 5-cyclopentyl-3-{2-hydroxy-5-[(2-methoxybenzoyl)amino]phenyl}-N-(2- |
| | methylbenzyl)-1H-pyrazole-1-carboxamide |
| 1521 | 5-cyclopentyl-3-{2-hydroxy-5-[(2-methoxybenzoyl)amino]phenyl}-N-(3- |
| | methylbenzyl)-1H-pyrazole-1-carboxamide |
| 1522 | 5-cyclopentyl-3-{2-hydroxy-5-[(2-methoxybenzoyl)amino]phenyl}-N-(4- |
| | methylbenzyl)-1H-pyrazole-1-carboxamide |
| 1523 | N-(2-chlorobenzyl)-5-cyclopentyl-3-{2-hydroxy-5-[(2- |
| | methoxybenzoyl)amino]phenyl}-1H-pyrazole-1-carboxamide |
| 1524 | 5-cyclopentyl-N-(4-fluorobenzyl)-3-{2-hydroxy-5-[(2- |
| | methoxybenzoyl)amino]phenyl}-1H-pyrazole-1-carboxamide |
| 1525 | 5-cyclopentyl-N-(3,4-dichlorobenzyl)-3-{2-hydroxy-5-[(2- |
| | methoxybenzoyl)amino]phenyl}-1H-pyrazole-1-carboxamide |
| 1526 | 5-cyclopentyl-3-{2-hydroxy-5-[(2-methoxybenzoyl)amino]phenyl}-N-(4- |
| | methoxybenzyl)-1H-pyrazole-1-carboxamide |
| 1527 | N-(sec-butyl)-5-cyclopentyl-3-{2-hydroxy-5-[(2-methoxybenzoyl)amino]phenyl}- |
| | 1H-pyrazole-1-carboxamide |
| 1528 | 5-cyclopentyl-N-(2-fluorobenzyl)-3-{2-hydroxy-5-[(2- |
| | methoxybenzoyl)amino]phenyl}-1H-pyrazole-1-carboxamide |
| 1529 | 5-cyclopentyl-N-(3-fluorobenzyl)-3-{2-hydroxy-5-[(2- |
| | methoxybenzoyl)amino]phenyl}-1H-pyrazole-1-carboxamide |
| 1530 | 5-cyclopentyl-N-cyclopropyl-3-{2-hydroxy-5-[(2- |
| | methoxybenzoyl)amino]phenyl}-1H-pyrazole-1-carboxamide |
| 1531 | 5-cyclopropyl-3-{2-hydroxy-5-[(2-methylbenzoyl)amino]phenyl}-N-isopropyl- |
| | 1H-pyrazole-1-carboxamide |
| 1532 | 5-cyclopropyl-N-ethyl-3-{2-hydroxy-5-[(2-methylbenzoyl)amino]phenyl}-1H- |
| | pyrazole-1-carboxamide |
| 1533 | N-allyl-5-cyclopropyl-3-{2-hydroxy-5-[(2-methylbenzoyl)amino]phenyl}-1H- |
| | pyrazole-1-carboxamide |
| 1534 | 5-cyclopropyl-3-{2-hydroxy-5-[(2-methylbenzoyl)amino]phenyl}-N-propyl-1H- |
| | pyrazole-1-carboxamide |
| 1535 | N-benzyl-5-cyclopropyl-3-{2-hydroxy-5-[(2-methylbenzoyl)amino]phenyl}-1H- |
| | pyrazole-1-carboxamide |
| 1536 | 5-cyclopropyl-3-{2-hydroxy-5-[(2-methylbenzoyl)amino]phenyl}-N-(2- |
| | phenylethyl)-1H-pyrazole-1-carboxamide |
| 1537 | 5-cyclopropyl-3-{2-hydroxy-5-[(2-methylbenzoyl)amino]phenyl}-N-(2- |
| | methylbenzyl)-1H-pyrazole-1-carboxamide |
| 1538 | 5-cyclopropyl-3-{2-hydroxy-5-[(2-methylbenzoyl)amino]phenyl}-N-(3- |
| | methylbenzyl)-1H-pyrazole-1-carboxamide |
| 1539 | 5-cyclopropyl-3-{2-hydroxy-5-[(2-methylbenzoyl)amino]phenyl}-N-(4- |
| | methylbenzyl)-1H-pyrazole-1-carboxamide |
| 1540 | N-(2-chlorobenzyl)-5-cyclopropyl-3-{2-hydroxy-5-[(2- |
| | methylbenzoyl)amino]phenyl}-1H-pyrazole-1-carboxamide |
| 1541 | 5-cyclopropyl-N-(4-fluorobenzyl)-3-{2-hydroxy-5-[(2- |
| | methylbenzoyl)amino]phenyl}-1H-pyrazole-1-carboxamide |
| 1542 | 5-cyclopropyl-N-(3,4-dichlorobenzyl)-3-{2-hydroxy-5-[(2- |
| | methylbenzoyl)amino]phenyl}-1H-pyrazole-1-carboxamide |
| 1543 | 5-cyclopropyl-3-{2-hydroxy-5-[(2-methylbenzoyl)amino]phenyl}-N-(4- |
| | methoxybenzyl)-1H-pyrazole-1-carboxamide |
| 1544 | N-(sec-butyl)-5-cyclopropyl-3-{2-hydroxy-5-[(2-methylbenzoyl)amino]phenyl}- |
| | 1H-pyrazole-1-carboxamide |
| 1545 | 5-cyclopropyl-N-(2-fluorobenzyl)-3-{2-hydroxy-5-[(2- |
| | methylbenzoyl)amino]phenyl}-1H-pyrazole-1-carboxamide |
| 1546 | 5-cyclobutyl-3-{2-hydroxy-5-[(2-methylbenzoyl)amino]phenyl}-N-isopropyl-1H- |
| | pyrazole-1-carboxamide |
| 1547 | N,5-dicyclopropyl-3-{2-hydroxy-5-[(2-methylbenzoyl)amino]phenyl}-1H- |
| | pyrazole-1-carboxamide |
| 1548 | 5-cyclobutyl-3-{2-hydroxy-5-[(2-methylbenzoyl)amino]phenyl}-N-isopropyl-1H- |
| | pyrazole-1-carboxamide |
| 1549 | 5-cyclobutyl-N-ethyl-3-{2-hydroxy-5-[(2-methylbenzoyl)amino]phenyl}-1H- |
| | pyrazole-1-carboxamide |
| 1550 | N-allyl-5-cyclobutyl-3-{2-hydroxy-5-[(2-methylbenzoyl)amino]phenyl}-1H- |
| | pyrazole-1-carboxamide |
| 1551 | 5-cyclobutyl-3-{2-hydroxy-5-[(2-methylbenzoyl)amino]phenyl}-N-propyl-1H- |
| | pyrazole-1-carboxamide |
| 1552 | N-benzyl-5-cyclobutyl-3-{2-hydroxy-5-[(2-methylbenzoyl)amino]phenyl}-1H- |
| | pyrazole-1-carboxamide |
| 1553 | 5-cyclobutyl-3-{2-hydroxy-5-[(2-methylbenzoyl)amino]phenyl}-N-(2- |
| | phenylethyl)-1H-pyrazole-1-carboxamide |
| 1554 | 5-cyclobutyl-3-{2-hydroxy-5-[(2-methylbenzoyl)amino]phenyl}-N-(2- |
| | methylbenzyl)-1H-pyrazole-1-carboxamide |
| 1555 | 5-cyclobutyl-3-{2-hydroxy-5-[(2-methylbenzoyl)amino]phenyl}-N-(3- |
| | methylbenzyl)-1H-pyrazole-1-carboxamide |
| 1556 | 5-cyclobutyl-3-{2-hydroxy-5-[(2-methylbenzoyl)amino]phenyl}-N-(4- |
| | methylbenzyl)-1H-pyrazole-1-carboxamide |
| 1557 | N-(2-chlorobenzyl)-5-cyclobutyl-3-{2-hydroxy-5-[(2- |
| | methylbenzoyl)amino]phenyl}-1H-pyrazole-1-carboxamide |
| 1558 | 5-cyclobutyl-N-(4-fluorobenzyl)-3-{2-hydroxy-5-[(2- |
| | methylbenzoyl)amino]phenyl}-1H-pyrazole-1-carboxamide |
| 1559 | 5-cyclobutyl-N-(3,4-dichlorobenzyl)-3-{2-hydroxy-5-[(2- |
| | methylbenzoyl)amino]phenyl}-1H-pyrazole-1-carboxamide |
| 1560 | 5-cyclobutyl-3-{2-hydroxy-5-[(2-methylbenzoyl)amino]phenyl}-N-(4- |
| | methoxybenzyl)-1H-pyrazole-1-carboxamide |
| 1561 | N-(sec-butyl)-5-cyclobutyl-3-{2-hydroxy-5-[(2-methylbenzoyl)amino]phenyl}- |
| | 1H-pyrazole-1-carboxamide |
| 1562 | 5-cyclobutyl-N-(2-fluorobenzyl)-3-{2-hydroxy-5-[(2- |
| | methylbenzoyl)amino]phenyl}-1H-pyrazole-1-carboxamide |
| 1563 | 5-cyclopentyl-3-{2-hydroxy-5-[(2-methylbenzoyl)amino]phenyl}-N-isopropyl- |
| | 1H-pyrazole-1-carboxamide |
| 1564 | 5-cyclobutyl-N-cyclopropyl-3-{2-hydroxy-5-[(2-methylbenzoyl)amino]phenyl}- |
| | 1H-pyrazole-1-carboxamide |
| 1565 | 5-cyclopentyl-3-{2-hydroxy-5-[(2-methylbenzoyl)amino]phenyl}-N-isopropyl- |
| | 1H-pyrazole-1-carboxamide |
| 1566 | 5-cyclopentyl-N-ethyl-3-{2-hydroxy-5-[(2-methylbenzoyl)amino]phenyl}-1H- |
| | pyrazole-1-carboxamide |
| 1567 | N-allyl-5-cyclopentyl-3-{2-hydroxy-5-[(2-methylbenzoyl)amino]phenyl}-1H- |
| | pyrazole-1-carboxamide |
| 1568 | 5-cyclopentyl-3-{2-hydroxy-5-[(2-methylbenzoyl)amino]phenyl}-N-propyl-1H- |
| | pyrazole-1-carboxamide |
| 1569 | N-benzyl-5-cyclopentyl-3-{2-hydroxy-5-[(2-methylbenzoyl)amino]phenyl}-1H- |
| | pyrazole-1-carboxamide |
| 1570 | 5-cyclopentyl-3-{2-hydroxy-5-[(2-methylbenzoyl)amino]phenyl}-N-(2- |
| | phenylethyl)-1H-pyrazole-1-carboxamide |
| 1571 | 5-cyclopentyl-3-{2-hydroxy-5-[(2-methylbenzoyl)amino]phenyl}-N-(2- |
| | methylbenzyl)-1H-pyrazole-1-carboxamide |
| 1572 | 5-cyclopentyl-3-{2-hydroxy-5-[(2-methylbenzoyl)amino]phenyl}-N-(3- |
| | methylbenzyl)-1H-pyrazole-1-carboxamide |
| 1573 | 5-cyclopentyl-3-{2-hydroxy-5-[(2-methylbenzoyl)amino]phenyl}-N-(4- |
| | methylbenzyl)-1H-pyrazole-1-carboxamide |
| 1574 | N-(2-chlorobenzyl)-5-cyclopentyl-3-{2-hydroxy-5-[(2- |
| | methylbenzoyl)amino]phenyl}-1H-pyrazole-1-carboxamide |
| 1575 | 5-cyclopentyl-N-(4-fluorobenzyl)-3-{2-hydroxy-5-[(2- |
| | methylbenzoyl)amino]phenyl}-1H-pyrazole-1-carboxamide |
| 1576 | 5-cyclopentyl-N-(3,4-dichlorobenzyl)-3-{2-hydroxy-5-[(2- |
| | methylbenzoyl)amino]phenyl}-1H-pyrazole-1-carboxamide |
| 1577 | 5-cyclopentyl-3-{2-hydroxy-5-[(2-methylbenzoyl)amino]phenyl}-N-(4- |
| | methoxybenzyl)-1H-pyrazole-1-carboxamide |
| 1578 | N-(sec-butyl)-5-cyclopentyl-3-{2-hydroxy-5-[(2-methylbenzoyl)amino]phenyl}- |
| | 1H-pyrazole-1-carboxamide |
| 1579 | 5-cyclopentyl-N-(2-fluorobenzyl)-3-{2-hydroxy-5-[(2- |
| | methylbenzoyl)amino]phenyl}-1H-pyrazole-1-carboxamide |
| 1580 | 5-cyclopentyl-N-(3-fluorobenzyl)-3-{2-hydroxy-5-[(2- |
| | methylbenzoyl)amino]phenyl}-1H-pyrazole-1-carboxamide |
| 1581 | 5-cyclopentyl-N-cyclopropyl-3-{2-hydroxy-5-[(2-methylbenzoyl)amino]phenyl}- |
| | 1H-pyrazole-1-carboxamide |
| 1582 | 5-cyclopropyl-3-(2-hydroxy-5-{[3-(trifluoromethyl)benzoyl]amino}phenyl)-N- |
| | isopropyl-1H-pyrazole-1-carboxamide |
| 1583 | 5-cyclopropyl-N-ethyl-3-(2-hydroxy-5-{[3- |
| | (trifluoromethyl)benzoyl]amino}phenyl)-1H-pyrazole-1-carboxamide |
| 1584 | N-allyl-5-cyclopropyl-3-(2-hydroxy-5-{[3- |
| | (trifluoromethyl)benzoyl]amino}phenyl)-1H-pyrazole-1-carboxamide |
| 1585 | 5-cyclopropyl-3-(2-hydroxy-5-{[3-(trifluoromethyl)benzoyl]amino}phenyl)-N- |
| | propyl-1H-pyrazole-1-carboxamide |
| 1586 | N-benzyl-5-cyclopropyl-3-(2-hydroxy-5-{[3- |
| | (trifluoromethyl)benzoyl]amino}phenyl)-1H-pyrazole-1-carboxamide |
| 1587 | 5-cyclopropyl-3-(2-hydroxy-5-{[3-(trifluoromethyl)benzoyl]amino}phenyl)-N-(2- |
| | phenylethyl)-1H-pyrazole-1-carboxamide |
| 1588 | 5-cyclopropyl-3-(2-hydroxy-5-{[3-(trifluoromethyl)benzoyl]amino}phenyl)-N-(2- |
| | methylbenzyl)-1H-pyrazole-1-carboxamide |
| 1589 | 5-cyclopropyl-3-(2-hydroxy-5-{[3-(trifluoromethyl)benzoyl]amino}phenyl)-N-(3- |
| | methylbenzyl)-1H-pyrazole-1-carboxamide |
| 1590 | 5-cyclopropyl-3-(2-hydroxy-5-{[3-(trifluoromethyl)benzoyl]amino}phenyl)-N-(4- |
| | methylbenzyl)-1H-pyrazole-1-carboxamide |
| 1591 | N-(2-chlorobenzyl)-5-cyclopropyl-3-(2-hydroxy-5-{[3- |
| | (trifluoromethyl)benzoyl]amino}phenyl)-1H-pyrazole-1-carboxamide |
| 1592 | 5-cyclopropyl-N-(4-fluorobenzyl)-3-(2-hydroxy-5-{[3- |
| | (trifluoromethyl)benzoyl]amino}phenyl)-1H-pyrazole-1-carboxamide |
| 1593 | 5-cyclopropyl-N-(3,4-dichlorobenzyl)-3-(2-hydroxy-5-{[3- |
| | (trifluoromethyl)benzoyl]amino}phenyl)-1H-pyrazole-1-carboxamide |
| 1594 | 5-cyclopropyl-3-(2-hydroxy-5-{[3-(trifluoromethyl)benzoyl]amino}phenyl)-N-(4- |
| | methoxybenzyl)-1H-pyrazole-1-carboxamide |
| 1595 | N-(sec-butyl)-5-cyclopropyl-3-(2-hydroxy-5-{[3- |
| | (trifluoromethyl)benzoyl]amino}phenyl)-1H-pyrazole-1-carboxamide |
| 1596 | 5-cyclopropyl-N-(2-fluorobenzyl)-3-(2-hydroxy-5-{[3- |
| | (trifluoromethyl)benzoyl]amino}phenyl)-1H-pyrazole-1-carboxamide |
| 1597 | 5-cyclopropyl-N-(3-fluorobenzyl)-3-(2-hydroxy-5-{[3- |
| | (trifluoromethyl)benzoyl]amino}phenyl)-1H-pyrazole-1-carboxamide |
| 1598 | N,5-dicyclopropyl-3-(2-hydroxy-5-{[3-(trifluoromethyl)benzoyl]amino}phenyl)- |
| | 1H-pyrazole-1-carboxamide |
| 1599 | 5-cyclobutyl-3-(2-hydroxy-5-{[3-(trifluoromethyl)benzoyl]amino}phenyl)-N- |
| | isopropyl-1H-pyrazole-1-carboxamide |
| 1600 | 5-cyclobutyl-N-ethyl-3-(2-hydroxy-5-{[3- |
| | (trifluoromethyl)benzoyl]amino}phenyl)-1H-pyrazole-1-carboxamide |
| 1601 | N-allyl-5-cyclobutyl-3-(2-hydroxy-5-{[3- |
| | (trifluoromethyl)benzoyl]amino}phenyl)-1H-pyrazole-1-carboxamide |
| 1602 | 5-cyclobutyl-3-(2-hydroxy-5-{[3-(trifluoromethyl)benzoyl]amino}phenyl)-N- |
| | propyl-1H-pyrazole-1-carboxamide |
| 1603 | N-benzyl-5-cyclobutyl-3-(2-hydroxy-5-{[3- |
| | (trifluoromethyl)benzoyl]amino}phenyl)-1H-pyrazole-1-carboxamide |
| 1604 | 5-cyclobutyl-3-(2-hydroxy-5-{[3-(trifluoromethyl)benzoyl]amino}phenyl)-N-(2- |
| | phenylethyl)-1H-pyrazole-1-carboxamide |
| 1605 | 5-cyclobutyl-3-(2-hydroxy-5-{[3-(trifluoromethyl)benzoyl]amino}phenyl)-N-(2- |
| | methylbenzyl)-1H-pyrazole-1-carboxamide |
| 1606 | 5-cyclobutyl-3-(2-hydroxy-5-{[3-(trifluoromethyl)benzoyl]amino}phenyl)-N-(3- |
| | methylbenzyl)-1H-pyrazole-1-carboxamide |
| 1607 | 5-cyclobutyl-3-(2-hydroxy-5-{[3-(trifluoromethyl)benzoyl]amino}phenyl)-N-(4- |
| | methylbenzyl)-1H-pyrazole-1-carboxamide |
| 1608 | N-(2-chlorobenzyl)-5-cyclobutyl-3-(2-hydroxy-5-{[3- |
| | (trifluoromethyl)benzoyl]amino}phenyl)-1H-pyrazole-1-carboxamide |
| 1609 | 5-cyclobutyl-N-(4-fluorobenzyl)-3-(2-hydroxy-5-{[3- |
| | (trifluoromethyl)benzoyl]amino}phenyl)-1H-pyrazole-1-carboxamide |
| 1610 | 5-cyclobutyl-N-(3,4-dichlorobenzyl)-3-(2-hydroxy-5-{[3- |
| | (trifluoromethyl)benzoyl]amino}phenyl)-1H-pyrazole-1-carboxamide |
| 1611 | 5-cyclobutyl-3-(2-hydroxy-5-{[3-(trifluoromethyl)benzoyl]amino}phenyl)-N-(4- |
| | methoxybenzyl)-1H-pyrazole-1-carboxamide |
| 1612 | N-(sec-butyl)-5-cyclobutyl-3-(2-hydroxy-5-{[3- |
| | (trifluoromethyl)benzoyl]amino}phenyl)-1H-pyrazole-1-carboxamide |
| 1613 | 5-cyclobutyl-N-(2-fluorobenzyl)-3-(2-hydroxy-5-{[3- |
| | (trifluoromethyl)benzoyl]amino}phenyl)-1H-pyrazole-1-carboxamide |
| 1614 | 5-cyclobutyl-N-(3-fluorobenzyl)-3-(2-hydroxy-5-{[3- |
| | (trifluoromethyl)benzoyl]amino}phenyl)-1H-pyrazole-1-carboxamide |
| 1615 | 5-cyclobutyl-N-cyclopropyl-3-(2-hydroxy-5-{[3- |
| | (trifluoromethyl)benzoyl]amino}phenyl)-1H-pyrazole-1-carboxamide |
| 1616 | 5-cyclopentyl-3-(2-hydroxy-5-{[3-(trifluoromethyl)benzoyl]amino}phenyl)-N- |
| | isopropyl-1H-pyrazole-1-carboxamide |
| 1617 | 5-cyclopentyl-N-ethyl-3-(2-hydroxy-5-{[3- |
| | (trifluoromethyl)benzoyl]amino}phenyl)-1H-pyrazole-1-carboxamide |
| 1618 | N-allyl-5-cyclopentyl-3-(2-hydroxy-5-{[3- |
| | (trifluoromethyl)benzoyl]amino}phenyl)-1H-pyrazole-1-carboxamide |
| 1619 | 5-cyclopentyl-3-(2-hydroxy-5-{[3-(trifluoromethyl)benzoyl]amino}phenyl)-N- |
| | propyl-1H-pyrazole-1-carboxamide |
| 1620 | N-benzyl-5-cyclopentyl-3-(2-hydroxy-5-{[3- |
| | (trifluoromethyl)benzoyl]amino}phenyl)-1H-pyrazole-1-carboxamide |
| 1621 | 5-cyclopentyl-3-(2-hydroxy-5-{[3-(trifluoromethyl)benzoyl]amino}phenyl)-N-(2- |
| | phenylethyl)-1H-pyrazole-1-carboxamide |
| 1622 | 5-cyclopentyl-3-(2-hydroxy-5-{[3-(trifluoromethyl)benzoyl]amino}phenyl)-N-(2- |
| | methylbenzyl)-1H-pyrazole-1-carboxamide |
| 1623 | 5-cyclopentyl-3-(2-hydroxy-5-{[3-(trifluoromethyl)benzoyl]amino}phenyl)-N-(3- |
| | methylbenzyl)-1H-pyrazole-1-carboxamide |
| 1624 | 5-cyclopentyl-3-(2-hydroxy-5-{[3-(trifluoromethyl)benzoyl]amino}phenyl)-N-(4- |
| | methylbenzyl)-1H-pyrazole-1-carboxamide |
| 1625 | N-(2-chlorobenzyl)-5-cyclopentyl-3-(2-hydroxy-5-{[3- |
| | (trifluoromethyl)benzoyl]amino}phenyl)-1H-pyrazole-1-carboxamide |
| 1626 | 5-cyclopentyl-N-(4-fluorobenzyl)-3-(2-hydroxy-5-{[3- |
| | (trifluoromethyl)benzoyl]amino}phenyl)-1H-pyrazole-1-carboxamide |
| 1627 | 5-cyclopentyl-N-(3,4-dichlorobenzyl)-3-(2-hydroxy-5-{[3- |
| | (trifluoromethyl)benzoyl]amino}phenyl)-1H-pyrazole-1-carboxamide |
| 1628 | 5-cyclopentyl-3-(2-hydroxy-5-{[3-(trifluoromethyl)benzoyl]amino}phenyl)-N-(4- |
| | methoxybenzyl)-1H-pyrazole-1-carboxamide |
| 1629 | N-(sec-butyl)-5-cyclopentyl-3-(2-hydroxy-5-{[3- |
| | (trifluoromethyl)benzoyl]amino}phenyl)-1H-pyrazole-1-carboxamide |
| 1630 | 5-cyclopentyl-N-(2-fluorobenzyl)-3-(2-hydroxy-5-{[3- |
| | (trifluoromethyl)benzoyl]amino}phenyl)-1H-pyrazole-1-carboxamide |
| 1631 | 5-cyclopentyl-N-(3-fluorobenzyl)-3-(2-hydroxy-5-{[3- |
| | (trifluoromethyl)benzoyl]amino}phenyl)-1H-pyrazole-1-carboxamide |
| 1632 | 5-cyclopentyl-N-cyclopropyl-3-(2-hydroxy-5-{[3- |
| | (trifluoromethyl)benzoyl]amino}phenyl)-1H-pyrazole-1-carboxamide |
| 1633 | 3-{5-[(4-chlorobenzoyl)amino]-2-hydroxyphenyl}-5-cyclopropyl-N-isopropyl-1H- |
| | pyrazole-1-carboxamide |
| 1634 | 3-{5-[(4-chlorobenzoyl)amino]-2-hydroxyphenyl}-5-cyclopropyl-N-ethyl-1H- |
| | pyrazole-1-carboxamide |
| 1635 | N-allyl-3-{5-[(4-chlorobenzoyl)amino]-2-hydroxyphenyl}-5-cyclopropyl-1H- |
| | pyrazole-1-carboxamide |
| 1636 | 3-{5-[(4-chlorobenzoyl)amino]-2-hydroxyphenyl}-5-cyclopropyl-N-propyl-1H- |
| | pyrazole-1-carboxamide |
| 1637 | N-benzyl-3-{5-[(4-chlorobenzoyl)amino]-2-hydroxyphenyl}-5-cyclopropyl-1H- |
| | pyrazole-1-carboxamide |
| 1638 | 3-{5-[(4-chlorobenzoyl)amino]-2-hydroxyphenyl}-5-cyclopropyl-N-(2- |
| | phenylethyl)-1H-pyrazole-1-carboxamide |
| 1639 | 3-{5-[(4-chlorobenzoyl)amino]-2-hydroxyphenyl}-5-cyclopropyl-N-(2- |
| | methylbenzyl)-1H-pyrazole-1-carboxamide |
| 1640 | 3-{5-[(4-chlorobenzoyl)amino]-2-hydroxyphenyl}-5-cyclopropyl-N-(3- |
| | methylbenzyl)-1H-pyrazole-1-carboxamide |
| 1641 | 3-{5-[(4-chlorobenzoyl)amino]-2-hydroxyphenyl}-5-cyclopropyl-N-(4- |
| | methylbenzyl)-1H-pyrazole-1-carboxamide |
| 1642 | 3-{5-[(4-chlorobenzoyl)amino]-2-hydroxyphenyl}-N-(2-chlorobenzyl)-5- |
| | cyclopropyl-1H-pyrazole-1-carboxamide |
| 1643 | 3-{5-[(4-chlorobenzoyl)amino]-2-hydroxyphenyl}-5-cyclopropyl-N-(4- |
| | fluorobenzyl)-1H-pyrazole-1-carboxamide |
| 1644 | 3-{5-[(4-chlorobenzoyl)amino]-2-hydroxyphenyl}-5-cyclopropyl-N-(3,4- |
| | dichlorobenzyl)-1H-pyrazole-1-carboxamide |
| 1645 | 3-{5-[(4-chlorobenzoyl)amino]-2-hydroxyphenyl}-5-cyclopropyl-N-(4- |
| | methoxybenzyl)-1H-pyrazole-1-carboxamide |
| 1646 | N-(sec-butyl)-3-{5-[(4-chlorobenzoyl)amino]-2-hydroxyphenyl}-5-cyclopropyl- |
| | 1H-pyrazole-1-carboxamide |
| 1647 | 3-{5-[(4-chlorobenzoyl)amino]-2-hydroxyphenyl}-5-cyclopropyl-N-(2- |
| | fluorobenzyl)-1H-pyrazole-1-carboxamide |
| 1648 | 3-{5-[(4-chlorobenzoyl)amino]-2-hydroxyphenyl}-5-cyclopropyl-N-(3- |
| | fluorobenzyl)-1H-pyrazole-1-carboxamide |
| 1649 | 3-{5-[(4-chlorobenzoyl)amino]-2-hydroxyphenyl}-N,5-dicyclopropyl-1H- |
| | pyrazole-1-carboxamide |
| 1650 | 3-{5-[(4-chlorobenzoyl)amino]-2-hydroxyphenyl}-5-cyclobutyl-N-isopropyl-1H- |
| | pyrazole-1-carboxamide |
| 1651 | 3-{5-[(4-chlorobenzoyl)amino]-2-hydroxyphenyl}-5-cyclobutyl-N-ethyl-1H- |
| | pyrazole-1-carboxamide |
| 1652 | N-allyl-3-{5-[(4-chlorobenzoyl)amino]-2-hydroxyphenyl}-5-cyclobutyl-1H- |
| | pyrazole-1-carboxamide |
| 1653 | 3-{5-[(4-chlorobenzoyl)amino]-2-hydroxyphenyl}-5-cyclobutyl-N-propyl-1H- |
| | pyrazole-1-carboxamide |
| 1654 | N-benzyl-3-{5-[(4-chlorobenzoyl)amino]-2-hydroxyphenyl}-5-cyclobutyl-1H- |
| | pyrazole-1-carboxamide |
| 1655 | 3-{5-[(4-chlorobenzoyl)amino]-2-hydroxyphenyl}-5-cyclobutyl-N-(2- |
| | phenylethyl)-1H-pyrazole-1-carboxamide |
| 1656 | 3-{5-[(4-chlorobenzoyl)amino]-2-hydroxyphenyl}-5-cyclobutyl-N-(2- |
| | methylbenzyl)-1H-pyrazole-1-carboxamide |
| 1657 | 3-{5-[(4-chlorobenzoyl)amino]-2-hydroxyphenyl}-5-cyclobutyl-N-(3- |
| | methylbenzyl)-1H-pyrazole-1-carboxamide |
| 1658 | 3-{5-[(4-chlorobenzoyl)amino]-2-hydroxyphenyl}-5-cyclobutyl-N-(4- |
| | methylbenzyl)-1H-pyrazole-1-carboxamide |
| 1659 | 3-{5-[(4-chlorobenzoyl)amino]-2-hydroxyphenyl}-N-(2-chlorobenzyl)-5- |
| | cyclobutyl-1H-pyrazole-1-carboxamide |
| 1660 | 3-{5-[(4-chlorobenzoyl)amino]-2-hydroxyphenyl}-5-cyclobutyl-N-(4- |
| | fluorobenzyl)-1H-pyrazole-1-carboxamide |
| 1661 | 3-{5-[(4-chlorobenzoyl)amino]-2-hydroxyphenyl}-5-cyclobutyl-N-(3,4- |
| | dichlorobenzyl)-1H-pyrazole-1-carboxamide |
| 1662 | 3-{5-[(4-chlorobenzoyl)amino]-2-hydroxyphenyl}-5-cyclobutyl-N-(4- |
| | methoxybenzyl)-1H-pyrazole-1-carboxamide |
| 1663 | N-(sec-butyl)-3-{5-[(4-chlorobenzoyl)amino]-2-hydroxyphenyl}-5-cyclobutyl-1H- |
| | pyrazole-1-carboxamide |
| 1664 | 3-{5-[(4-chlorobenzoyl)amino]-2-hydroxyphenyl}-5-cyclobutyl-N-(2- |
| | fluorobenzyl)-1H-pyrazole-1-carboxamide |
| 1665 | 3-{5-[(4-chlorobenzoyl)amino]-2-hydroxyphenyl}-5-cyclobutyl-N-(3- |
| | fluorobenzyl)-1H-pyrazole-1-carboxamide |
| 1666 | 3-{5-[(4-chlorobenzoyl)amino]-2-hydroxyphenyl}-5-cyclobutyl-N-cyclopropyl- |
| | 1H-pyrazole-1-carboxamide |
| 1667 | 3-{5-[(4-chlorobenzoyl)amino]-2-hydroxyphenyl}-5-cyclopentyl-N-isopropyl-1H- |
| | pyrazole-1-carboxamide |
| 1668 | 3-{5-[(4-chlorobenzoyl)amino]-2-hydroxyphenyl}-5-cyclopentyl-N-ethyl-1H- |
| | pyrazole-1-carboxamide |
| 1669 | N-allyl-3-{5-[(4-chlorobenzoyl)amino]-2-hydroxyphenyl}-5-cyclopentyl-1H- |
| | pyrazole-1-carboxamide |
| 1670 | 3-{5-[(4-chlorobenzoyl)amino]-2-hydroxyphenyl}-5-cyclopentyl-N-propyl-1H- |
| | pyrazole-1-carboxamide |
| 1671 | N-benzyl-3-{5-[(4-chlorobenzoyl)amino]-2-hydroxyphenyl}-5-cyclopentyl-1H- |
| | pyrazole-1-carboxamide |
| 1672 | 3-{5-[(4-chlorobenzoyl)amino]-2-hydroxyphenyl}-5-cyclopentyl-N-(2- |
| | phenylethyl)-1H-pyrazole-1-carboxamide |
| 1673 | 3-{5-[(4-chlorobenzoyl)amino]-2-hydroxyphenyl}-5-cyclopentyl-N-(2- |
| | methylbenzyl)-1H-pyrazole-1-carboxamide |
| 1674 | 3-{5-[(4-chlorobenzoyl)amino]-2-hydroxyphenyl}-5-cyclopentyl-N-(3- |
| | methylbenzyl)-1H-pyrazole-1-carboxamide |
| 1675 | 3-{5-[(4-chlorobenzoyl)amino]-2-hydroxyphenyl}-5-cyclopentyl-N-(4- |
| | methylbenzyl)-1H-pyrazole-1-carboxamide |
| 1676 | 3-{5-[(4-chlorobenzoyl)amino]-2-hydroxyphenyl}-N-(2-chlorobenzyl)-5- |
| | cyclopentyl-1H-pyrazole-1-carboxamide |
| 1677 | 3-{5-[(4-chlorobenzoyl)amino]-2-hydroxyphenyl}-5-cyclopentyl-N-(4- |
| | fluorobenzyl)-1H-pyrazole-1-carboxamide |
| 1678 | 3-{5-[(4-chlorobenzoyl)amino]-2-hydroxyphenyl}-5-cyclopentyl-N-(3,4- |
| | dichlorobenzyl)-1H-pyrazole-1-carboxamide |
| 1679 | 3-{5-[(4-chlorobenzoyl)amino]-2-hydroxyphenyl}-5-cyclopentyl-N-(4- |
| | methoxybenzyl)-1H-pyrazole-1-carboxamide |
| 1680 | N-(sec-butyl)-3-{5-[(4-chlorobenzoyl)amino]-2-hydroxyphenyl}-5-cyclopentyl- |
| | 1H-pyrazole-1-carboxamide |
| 1681 | 3-{5-[(4-chlorobenzoyl)amino]-2-hydroxyphenyl}-5-cyclopentyl-N-(2- |
| | fluorobenzyl)-1H-pyrazole-1-carboxamide |
| 1682 | 3-{5-[(4-chlorobenzoyl)amino]-2-hydroxyphenyl}-5-cyclopentyl-N-(3- |
| | fluorobenzyl)-1H-pyrazole-1-carboxamide |
| 1683 | 3-{5-[(4-chlorobenzoyl)amino]-2-hydroxyphenyl}-5-cyclopentyl-N-cyclopropyl- |
| | 1H-pyrazole-1-carboxamide |
| 1684 | 5-cyclopropyl-3-{5-[(4-ethoxybenzoyl)amino]-2-hydroxyphenyl}-N-isopropyl- |
| | 1H-pyrazole-1-carboxamide |
| 1685 | 5-cyclopropyl-3-{5-[(4-ethoxybenzoyl)amino]-2-hydroxyphenyl}-N-ethyl-1H- |
| | pyrazole-1-carboxamide |
| 1686 | N-allyl-5-cyclopropyl-3-{5-[(4-ethoxybenzoyl)amino]-2-hydroxyphenyl}-1H- |
| | pyrazole-1-carboxamide |
| 1687 | 5-cyclopropyl-3-{5-[(4-ethoxybenzoyl)amino]-2-hydroxyphenyl}-N-propyl-1H- |
| | pyrazole-1-carboxamide |
| 1688 | N-benzyl-5-cyclopropyl-3-{5-[(4-ethoxybenzoyl)amino]-2-hydroxyphenyl}-1H- |
| | pyrazole-1-carboxamide |
| 1689 | 5-cyclopropyl-3-{5-[(4-ethoxybenzoyl)amino]-2-hydroxyphenyl}-N-(2- |
| | phenylethyl)-1H-pyrazole-1-carboxamide |
| 1690 | 5-cyclopropyl-3-{5-[(4-ethoxybenzoyl)amino]-2-hydroxyphenyl}-N-(2- |
| | methylbenzyl)-1H-pyrazole-1-carboxamide |
| 1691 | 5-cyclopropyl-3-{5-[(4-ethoxybenzoyl)amino]-2-hydroxyphenyl}-N-(3- |
| | methylbenzyl)-1H-pyrazole-1-carboxamide |
| 1692 | 5-cyclopropyl-3-{5-[(4-ethoxybenzoyl)amino]-2-hydroxyphenyl}-N-(4- |
| | methylbenzyl)-1H-pyrazole-1-carboxamide |
| 1693 | N-(2-chlorobenzyl)-5-cyclopropyl-3-{5-[(4-ethoxybenzoyl)amino]-2- |
| | hydroxyphenyl}-1H-pyrazole-1-carboxamide |
| 1694 | 5-cyclopropyl-3-{5-[(4-ethoxybenzoyl)amino]-2-hydroxyphenyl}-N-(4- |
| | fluorobenzyl)-1H-pyrazole-1-carboxamide |
| 1695 | 5-cyclopropyl-N-(3,4-dichlorobenzyl)-3-{5-[(4-ethoxybenzoyl)amino]-2- |
| | hydroxyphenyl}-1H-pyrazole-1-carboxamide |
| 1696 | 5-cyclopropyl-3-{5-[(4-ethoxybenzoyl)amino]-2-hydroxyphenyl}-N-(4- |
| | methoxybenzyl)-1H-pyrazole-1-carboxamide |
| 1697 | N-(sec-butyl)-5-cyclopropyl-3-{5-[(4-ethoxybenzoyl)amino]-2-hydroxyphenyl}- |
| | 1H-pyrazole-1-carboxamide |
| 1698 | 5-cyclopropyl-3-{5-[(4-ethoxybenzoyl)amino]-2-hydroxyphenyl}-N-(2- |
| | fluorobenzyl)-1H-pyrazole-1-carboxamide |
| 1699 | 5-cyclopropyl-3-{5-[(4-ethoxybenzoyl)amino]-2-hydroxyphenyl}-N-(3- |
| | fluorobenzyl)-1H-pyrazole-1-carboxamide |
| 1700 | N,5-dicyclopropyl-3-{5-[(4-ethoxybenzoyl)amino]-2-hydroxyphenyl}-1H- |
| | pyrazole-1-carboxamide |
| 1701 | 5-cyclobutyl-3-{5-[(4-ethoxybenzoyl)amino]-2-hydroxyphenyl}-N-isopropyl-1H- |
| | pyrazole-1-carboxamide |
| 1702 | 5-cyclobutyl-3-{5-[(4-ethoxybenzoyl)amino]-2-hydroxyphenyl}-N-ethyl-1H- |
| | pyrazole-1-carboxamide |
| 1703 | N-allyl-5-cyclobutyl-3-{5-[(4-ethoxybenzoyl)amino]-2-hydroxyphenyl}-1H- |
| | pyrazole-1-carboxamide |
| 1704 | 5-cyclobutyl-3-{5-[(4-ethoxybenzoyl)amino]-2-hydroxyphenyl}-N-propyl-1H- |
| | pyrazole-1-carboxamide |
| 1705 | N-benzyl-5-cyclobutyl-3-{5-[(4-ethoxybenzoyl)amino]-2-hydroxyphenyl}-1H- |
| | pyrazole-1-carboxamide |
| 1706 | 5-cyclobutyl-3-{5-[(4-ethoxybenzoyl)amino]-2-hydroxyphenyl}-N-(2- |
| | phenylethyl)-1H-pyrazole-1-carboxamide |
| 1707 | 5-cyclobutyl-3-{5-[(4-ethoxybenzoyl)amino]-2-hydroxyphenyl}-N-(2- |
| | methylbenzyl)-1H-pyrazole-1-carboxamide |
| 1708 | 5-cyclobutyl-3-{5-[(4-ethoxybenzoyl)amino]-2-hydroxyphenyl}-N-(3- |
| | methylbenzyl)-1H-pyrazole-1-carboxamide |
| 1709 | 5-cyclobutyl-3-{5-[(4-ethoxybenzoyl)amino]-2-hydroxyphenyl}-N-(4- |
| | methylbenzyl)-1H-pyrazole-1-carboxamide |
| 1710 | N-(2-chlorobenzyl)-5-cyclobutyl-3-{5-[(4-ethoxybenzoyl)amino]-2- |
| | hydroxyphenyl}-1H-pyrazole-1-carboxamide |
| 1711 | 5-cyclobutyl-3-{5-[(4-ethoxybenzoyl)amino]-2-hydroxyphenyl}-N-(4- |
| | fluorobenzyl)-1H-pyrazole-1-carboxamide |
| 1712 | 5-cyclobutyl-N-(3,4-dichlorobenzyl)-3-{5-[(4-ethoxybenzoyl)amino]-2- |
| | hydroxyphenyl}-1H-pyrazole-1-carboxamide |
| 1713 | 5-cyclobutyl-3-{5-[(4-ethoxybenzoyl)amino]-2-hydroxyphenyl}-N-(4- |
| | methoxybenzyl)-1H-pyrazole-1-carboxamide |
| 1714 | N-(sec-butyl)-5-cyclobutyl-3-{5-[(4-ethoxybenzoyl)amino]-2-hydroxyphenyl}- |
| | 1H-pyrazole-1-carboxamide |
| 1715 | 5-cyclobutyl-3-{5-[(4-ethoxybenzoyl)amino]-2-hydroxyphenyl}-N-(2- |
| | fluorobenzyl)-1H-pyrazole-1-carboxamide |
| 1716 | 5-cyclobutyl-3-{5-[(4-ethoxybenzoyl)amino]-2-hydroxyphenyl}-N-(3- |
| | fluorobenzyl)-1H-pyrazole-1-carboxamide |
| 1717 | 5-cyclobutyl-N-cyclopropyl-3-{5-[(4-ethoxybenzoyl)amino]-2-hydroxyphenyl}- |
| | 1H-pyrazole-1-carboxamide |
| 1718 | 5-cyclopentyl-3-{5-[(4-ethoxybenzoyl)amino]-2-hydroxyphenyl}-N-isopropyl-1H- |
| | pyrazole-1-carboxamide |
| 1719 | 5-cyclopentyl-3-{5-[(4-ethoxybenzoyl)amino]-2-hydroxyphenyl}-N-ethyl-1H- |
| | pyrazole-1-carboxamide |
| 1720 | N-allyl-5-cyclopentyl-3-{5-[(4-ethoxybenzoyl)amino]-2-hydroxyphenyl}-1H- |
| | pyrazole-1-carboxamide |
| 1721 | 5-cyclopentyl-3-{5-[(4-ethoxybenzoyl)amino]-2-hydroxyphenyl}-N-propyl-1H- |
| | pyrazole-1-carboxamide |
| 1722 | N-benzyl-5-cyclopentyl-3-{5-[(4-ethoxybenzoyl)amino]-2-hydroxyphenyl}-1H- |
| | pyrazole-1-carboxamide |
| 1723 | 5-cyclopentyl-3-{5-[(4-ethoxybenzoyl)amino]-2-hydroxyphenyl}-N-(2- |
| | phenylethyl)-1H-pyrazole-1-carboxamide |
| 1724 | 5-cyclopentyl-3-{5-[(4-ethoxybenzoyl)amino]-2-hydroxyphenyl}-N-(2- |
| | methylbenzyl)-1H-pyrazole-1-carboxamide |
| 1725 | 5-cyclopentyl-3-{5-[(4-ethoxybenzoyl)amino]-2-hydroxyphenyl}-N-(3- |
| | methylbenzyl)-1H-pyrazole-1-carboxamide |
| 1726 | 5-cyclopentyl-3-{5-[(4-ethoxybenzoyl)amino]-2-hydroxyphenyl}-N-(4- |
| | methylbenzyl)-1H-pyrazole-1-carboxamide |
| 1727 | N-(2-chlorobenzyl)-5-cyclopentyl-3-{5-[(4-ethoxybenzoyl)amino]-2- |
| | hydroxyphenyl}-1H-pyrazole-1-carboxamide |
| 1728 | 5-cyclopentyl-3-{5-[(4-ethoxybenzoyl)amino]-2-hydroxyphenyl}-N-(4- |
| | fluorobenzyl)-1H-pyrazole-1-carboxamide |
| 1729 | 5-cyclopentyl-N-(3,4-dichlorobenzyl)-3-{5-[(4-ethoxybenzoyl)amino]-2- |
| | hydroxyphenyl}-1H-pyrazole-1-carboxamide |
| 1730 | 5-cyclopentyl-3-{5-[(4-ethoxybenzoyl)amino]-2-hydroxyphenyl}-N-(4- |
| | methoxybenzyl)-1H-pyrazole-1-carboxamide |
| 1731 | N-(sec-butyl)-5-cyclopentyl-3-{5-[(4-ethoxybenzoyl)amino]-2-hydroxyphenyl}- |
| | 1H-pyrazole-1-carboxamide |
| 1732 | 5-cyclopentyl-3-{5-[(4-ethoxybenzoyl)amino]-2-hydroxyphenyl}-N-(2- |
| | fluorobenzyl)-1H-pyrazole-1-carboxamide |
| 1733 | 5-cyclopentyl-3-{5-[(4-ethoxybenzoyl)amino]-2-hydroxyphenyl}-N-(3- |
| | fluorobenzyl)-1H-pyrazole-1-carboxamide |
| 1734 | 5-cyclopentyl-N-cyclopropyl-3-{5-[(4-ethoxybenzoyl)amino]-2-hydroxyphenyl}- |
| | 1H-pyrazole-1-carboxamide |
| 1735 | 3-{5-[(4-tert-butylbenzoyl)amino]-2-hydroxyphenyl}-5-cyclopropyl-N-isopropyl- |
| | 1H-pyrazole-1-carboxamide |
| 1736 | 3-{5-[(4-tert-butylbenzoyl)amino]-2-hydroxyphenyl}-5-cyclopropyl-N-ethyl-1H- |
| | pyrazole-1-carboxamide |
| 1737 | N-allyl-3-{5-[(4-tert-butylbenzoyl)amino]-2-hydroxyphenyl}-5-cyclopropyl-1H- |
| | pyrazole-1-carboxamide |
| 1738 | 3-{5-[(4-tert-butylbenzoyl)amino]-2-hydroxyphenyl}-5-cyclopropyl-N-propyl-1H- |
| | pyrazole-1-carboxamide |
| 1739 | N-benzyl-3-{5-[(4-tert-butylbenzoyl)amino]-2-hydroxyphenyl}-5-cyclopropyl-1H- |
| | pyrazole-1-carboxamide |
| 1740 | 3-{5-[(4-tert-butylbenzoyl)amino]-2-hydroxyphenyl}-5-cyclopropyl-N-(2- |
| | phenylethyl)-1H-pyrazole-1-carboxamide |
| 1741 | 3-{5-[(4-tert-butylbenzoyl)amino]-2-hydroxyphenyl}-5-cyclopropyl-N-(2- |
| | methylbenzyl)-1H-pyrazole-1-carboxamide |
| 1742 | 3-{5-[(4-tert-butylbenzoyl)amino]-2-hydroxyphenyl}-5-cyclopropyl-N-(3- |
| | methylbenzyl)-1H-pyrazole-1-carboxamide |
| 1743 | 3-{5-[(4-tert-butylbenzoyl)amino]-2-hydroxyphenyl}-5-cyclopropyl-N-(4- |
| | methylbenzyl)-1H-pyrazole-1-carboxamide |
| 1744 | 3-{5-[(4-tert-butylbenzoyl)amino]-2-hydroxyphenyl}-N-(2-chlorobenzyl)-5- |
| | cyclopropyl-1H-pyrazole-1-carboxamide |
| 1745 | 3-{5-[(4-tert-butylbenzoyl)amino]-2-hydroxyphenyl}-5-cyclopropyl-N-(4- |
| | fluorobenzyl)-1H-pyrazole-1-carboxamide |
| 1746 | 3-{5-[(4-tert-butylbenzoyl)amino]-2-hydroxyphenyl}-5-cyclopropyl-N-(3,4- |
| | dichlorobenzyl)-1H-pyrazole-1-carboxamide |
| 1747 | 3-{5-[(4-tert-butylbenzoyl)amino]-2-hydroxyphenyl}-5-cyclopropyl-N-(4- |
| | methoxybenzyl)-1H-pyrazole-1-carboxamide |
| 1748 | N-(sec-butyl)-3-{5-[(4-tert-butylbenzoyl)amino]-2-hydroxyphenyl}-5- |
| | cyclopropyl-1H-pyrazole-1-carboxamide |
| 1749 | 3-{5-[(4-tert-butylbenzoyl)amino]-2-hydroxyphenyl}-5-cyclopropyl-N-(2- |
| | fluorobenzyl)-1H-pyrazole-1-carboxamide |
| 1750 | 3-{5-[(4-tert-butylbenzoyl)amino]-2-hydroxyphenyl}-5-cyclopropyl-N-(3- |
| | fluorobenzyl)-1H-pyrazole-1-carboxamide |
| 1751 | 3-{5-[(4-tert-butylbenzoyl)amino]-2-hydroxyphenyl}-N,5-dicyclopropyl-1H- |
| | pyrazole-1-carboxamide |
| 1752 | 3-{5-[(4-tert-butylbenzoyl)amino]-2-hydroxyphenyl}-5-cyclobutyl-N-isopropyl- |
| | 1H-pyrazole-1-carboxamide |
| 1753 | 3-{5-[(4-tert-butylbenzoyl)amino]-2-hydroxyphenyl}-5-cyclobutyl-N-ethyl-1H- |
| | pyrazole-1-carboxamide |
| 1754 | N-allyl-3-{5-[(4-tert-butylbenzoyl)amino]-2-hydroxyphenyl}-5-cyclobutyl-1H- |
| | pyrazole-1-carboxamide |
| 1755 | 3-{5-[(4-tert-butylbenzoyl)amino]-2-hydroxyphenyl}-5-cyclobutyl-N-propyl-1H- |
| | pyrazole-1-carboxamide |
| 1756 | N-benzyl-3-{5-[(4-tert-butylbenzoyl)amino]-2-hydroxyphenyl}-5-cyclobutyl-1H- |
| | pyrazole-1-carboxamide |
| 1757 | 3-{5-[(4-tert-butylbenzoyl)amino]-2-hydroxyphenyl}-5-cyclobutyl-N-(2- |
| | phenylethyl)-1H-pyrazole-1-carboxamide |
| 1758 | 3-{5-[(4-tert-butylbenzoyl)amino]-2-hydroxyphenyl}-5-cyclobutyl-N-(2- |
| | methylbenzyl)-1H-pyrazole-1-carboxamide |
| 1759 | 3-{5-[(4-tert-butylbenzoyl)amino]-2-hydroxyphenyl}-5-cyclobutyl-N-(3- |
| | methylbenzyl)-1H-pyrazole-1-carboxamide |
| 1760 | 3-{5-[(4-tert-butylbenzoyl)amino]-2-hydroxyphenyl}-5-cyclobutyl-N-(4- |
| | methylbenzyl)-1H-pyrazole-1-carboxamide |
| 1761 | 3-{5-[(4-tert-butylbenzoyl)amino]-2-hydroxyphenyl}-N-(2-chlorobenzyl)-5- |
| | cyclobutyl-1H-pyrazole-1-carboxamide |
| 1762 | 3-{5-[(4-tert-butylbenzoyl)amino]-2-hydroxyphenyl}-5-cyclobutyl-N-(4- |
| | fluorobenzyl)-1H-pyrazole-1-carboxamide |
| 1763 | 3-{5-[(4-tert-butylbenzoyl)amino]-2-hydroxyphenyl}-5-cyclobutyl-N-(3,4- |
| | dichlorobenzyl)-1H-pyrazole-1-carboxamide |
| 1764 | 3-{5-[(4-tert-butylbenzoyl)amino]-2-hydroxyphenyl}-5-cyclobutyl-N-(4- |
| | methoxybenzyl)-1H-pyrazole-1-carboxamide |
| 1765 | N-(sec-butyl)-3-{5-[(4-tert-butylbenzoyl)amino]-2-hydroxyphenyl}-5-cyclobutyl- |
| | 1H-pyrazole-1-carboxamide |
| 1766 | 3-{5-[(4-tert-butylbenzoyl)amino]-2-hydroxyphenyl}-5-cyclobutyl-N-(2- |
| | fluorobenzyl)-1H-pyrazole-1-carboxamide |
| 1767 | 3-{5-[(4-tert-butylbenzoyl)amino]-2-hydroxyphenyl}-5-cyclobutyl-N-cyclopropyl- |
| | 1H-pyrazole-1-carboxamide |
| 1768 | 3-{5-[(4-tert-butylbenzoyl)amino]-2-hydroxyphenyl}-5-cyclobutyl-N-cyclopropyl- |
| | 1H-pyrazole-1-carboxamide |
| 1769 | 3-{5-[(4-tert-butylbenzoyl)amino]-2-hydroxyphenyl}-5-cyclopentyl-N-isopropyl- |
| | 1H-pyrazole-1-carboxamide |
| 1770 | 3-{5-[(4-tert-butylbenzoyl)amino]-2-hydroxyphenyl}-5-cyclopentyl-N-ethyl-1H- |
| | pyrazole-1-carboxamide |
| 1771 | N-allyl-3-{5-[(4-tert-butylbenzoyl)amino]-2-hydroxyphenyl}-5-cyclopentyl-1H- |
| | pyrazole-1-carboxamide |
| 1772 | 3-{5-[(4-tert-butylbenzoyl)amino]-2-hydroxyphenyl}-5-cyclopentyl-N-propyl-1H- |
| | pyrazole-1-carboxamide |
| 1773 | N-benzyl-3-{5-[(4-tert-butylbenzoyl)amino]-2-hydroxyphenyl}-5-cyclopentyl-1H- |
| | pyrazole-1-carboxamide |
| 1774 | 3-{5-[(4-tert-butylbenzoyl)amino]-2-hydroxyphenyl}-5-cyclopentyl-N-(2- |
| | phenylethyl)-1H-pyrazole-1-carboxamide |
| 1775 | 3-{5-[(4-tert-butylbenzoyl)amino]-2-hydroxyphenyl}-5-cyclopentyl-N-(2- |
| | methylbenzyl)-1H-pyrazole-1-carboxamide |
| 1776 | 3-{5-[(4-tert-butylbenzoyl)amino]-2-hydroxyphenyl}-5-cyclopentyl-N-(3- |
| | methylbenzyl)-1H-pyrazole-1-carboxamide |
| 1777 | 3-{5-[(4-tert-butylbenzoyl)amino]-2-hydroxyphenyl}-5-cyclopentyl-N-(4- |
| | methylbenzyl)-1H-pyrazole-1-carboxamide |
| 1778 | 3-{5-[(4-tert-butylbenzoyl)amino]-2-hydroxyphenyl}-N-(2-chlorobenzyl)-5- |
| | cyclopentyl-1H-pyrazole-1-carboxamide |
| 1779 | 3-{5-[(4-tert-butylbenzoyl)amino]-2-hydroxyphenyl}-5-cyclopentyl-N-(4- |
| | fluorobenzyl)-1H-pyrazole-1-carboxamide |
| 1780 | 3-{5-[(4-tert-butylbenzoyl)amino]-2-hydroxyphenyl}-5-cyclopentyl-N-(3,4- |
| | dichlorobenzyl)-1H-pyrazole-1-carboxamide |
| 1781 | 3-{5-[(4-tert-butylbenzoyl)amino]-2-hydroxyphenyl}-5-cyclopentyl-N-(4- |
| | methoxybenzyl)-1H-pyrazole-1-carboxamide |
| 1782 | N-(sec-butyl)-3-{5-[(4-tert-butylbenzoyl)amino]-2-hydroxyphenyl}-5-cyclopentyl- |
| | 1H-pyrazole-1-carboxamide |
| 1783 | 3-{5-[(4-tert-butylbenzoyl)amino]-2-hydroxyphenyl}-5-cyclopentyl-N-(2- |
| | fluorobenzyl)-1H-pyrazole-1-carboxamide |
| 1784 | 3-{5-[(4-tert-butylbenzoyl)amino]-2-hydroxyphenyl}-5-cyclopentyl-N-(3- |
| | fluorobenzyl)-1H-pyrazole-1-carboxamide |
| 1785 | 3-{5-[(4-tert-butylbenzoyl)amino]-2-hydroxyphenyl}-5-cyclopentyl-N- |
| | cyclopropyl-1H-pyrazole-1-carboxamide |
| 1786 | 5-cyclopropyl-3-{2-hydroxy-5-[(4-methylbenzoyl)amino]phenyl}-N-isopropyl- |
| | 1H-pyrazole-1-carboxamide |
| 1787 | 5-cyclopropyl-N-ethyl-3-{2-hydroxy-5-[(4-methylbenzoyl)amino]phenyl}-1H- |
| | pyrazole-1-carboxamide |
| 1788 | N-allyl-5-cyclopropyl-3-{2-hydroxy-5-[(4-methylbenzoyl)amino]phenyl}-1H- |
| | pyrazole-1-carboxamide |
| 1789 | 5-cyclopropyl-3-{2-hydroxy-5-[(4-methylbenzoyl)amino]phenyl}-N-propyl-1H- |
| | pyrazole-1-carboxamide |
| 1790 | N-benzyl-5-cyclopropyl-3-{2-hydroxy-5-[(4-methylbenzoyl)amino]phenyl}-1H- |
| | pyrazole-1-carboxamide |
| 1791 | 5-cyclopropyl-3-{2-hydroxy-5-[(4-methylbenzoyl)amino]phenyl}-N-(2- |
| | phenylethyl)-1H-pyrazole-1-carboxamide |
| 1792 | 5-cyclopropyl-3-{2-hydroxy-5-[(4-methylbenzoyl)amino]phenyl}-N-(2- |
| | methylbenzyl)-1H-pyrazole-1-carboxamide |
| 1793 | 5-cyclopropyl-3-{2-hydroxy-5-[(4-methylbenzoyl)amino]phenyl}-N-(3- |
| | methylbenzyl)-1H-pyrazole-1-carboxamide |
| 1794 | 5-cyclopropyl-3-{2-hydroxy-5-[(4-methylbenzoyl)amino]phenyl}-N-(4- |
| | methylbenzyl)-1H-pyrazole-1-carboxamide |
| 1795 | N-(2-chlorobenzyl)-5-cyclopropyl-3-{2-hydroxy-5-[(4- |
| | methylbenzoyl)amino]phenyl}-1H-pyrazole-1-carboxamide |
| 1796 | 5-cyclopropyl-N-(4-fluorobenzyl)-3-{2-hydroxy-5-[(4- |
| | methylbenzoyl)amino]phenyl}-1H-pyrazole-1-carboxamide |
| 1797 | 5-cyclopropyl-N-(3,4-dichlorobenzyl)-3-{2-hydroxy-5-[(4- |
| | methylbenzoyl)amino]phenyl}-1H-pyrazole-1-carboxamide |
| 1798 | 5-cyclopropyl-3-{2-hydroxy-5-[(4-methylbenzoyl)amino]phenyl}-N-(4- |
| | methoxybenzyl)-1H-pyrazole-1-carboxamide |
| 1799 | N-(sec-butyl)-5-cyclopropyl-3-{2-hydroxy-5-[(4-methylbenzoyl)amino]phenyl}- |
| | 1H-pyrazole-1-carboxamide |
| 1800 | 5-cyclopropyl-N-(2-fluorobenzyl)-3-{2-hydroxy-5-[(4- |
| | methylbenzoyl)amino]phenyl}-1H-pyrazole-1-carboxamide |
| 1801 | 5-cyclopropyl-N-(3-fluorobenzyl)-3-{2-hydroxy-5-[(4- |
| | methylbenzoyl)amino]phenyl}-1H-pyrazole-1-carboxamide |
| 1802 | N,5-dicyclopropyl-3-{2-hydroxy-5-[(4-methylbenzoyl)amino]phenyl}-1H- |
| | pyrazole-1-carboxamide |
| 1803 | 5-cyclobutyl-3-{2-hydroxy-5-[(4-methylbenzoyl)amino]phenyl}-N-isopropyl-1H- |
| | pyrazole-1-carboxamide |
| 1804 | 5-cyclobutyl-N-ethyl-3-{2-hydroxy-5-[(4-methylbenzoyl)amino]phenyl}-1H- |
| | pyrazole-1-carboxamide |
| 1805 | N-allyl-5-cyclobutyl-3-{2-hydroxy-5-[(4-methylbenzoyl)amino]phenyl}-1H- |
| | pyrazole-1-carboxamide |
| 1806 | 5-cyclobutyl-3-{2-hydroxy-5-[(4-methylbenzoyl)amino]phenyl}-N-propyl-1H- |
| | pyrazole-1-carboxamide |
| 1807 | N-benzyl-5-cyclobutyl-3-{2-hydroxy-5-[(4-methylbenzoyl)amino]phenyl}-1H- |
| | pyrazole-1-carboxamide |
| 1808 | 5-cyclobutyl-3-{2-hydroxy-5-[(4-methylbenzoyl)amino]phenyl}-N-(2- |
| | phenylethyl)-1H-pyrazole-1-carboxamide |
| 1809 | 5-cyclobutyl-3-{2-hydroxy-5-[(4-methylbenzoyl)amino]phenyl}-N-(2- |
| | methylbenzyl)-1H-pyrazole-1-carboxamide |
| 1810 | 5-cyclobutyl-3-{2-hydroxy-5-[(4-methylbenzoyl)amino]phenyl}-N-(3- |
| | methylbenzyl)-1H-pyrazole-1-carboxamide |
| 1811 | 5-cyclobutyl-3-{2-hydroxy-5-[(4-methylbenzoyl)amino]phenyl}-N-(4- |
| | methylbenzyl)-1H-pyrazole-1-carboxamide |
| 1812 | N-(2-chlorobenzyl)-5-cyclobutyl-3-{2-hydroxy-5-[(4- |
| | methylbenzoyl)amino]phenyl}-1H-pyrazole-1-carboxamide |
| 1813 | 5-cyclobutyl-N-(4-fluorobenzyl)-3-{2-hydroxy-5-[(4- |
| | methylbenzoyl)amino]phenyl}-1H-pyrazole-1-carboxamide |
| 1814 | 5-cyclobutyl-N-(3,4-dichlorobenzyl)-3-{2-hydroxy-5-[(4- |
| | methylbenzoyl)amino]phenyl}-1H-pyrazole-1-carboxamide |
| 1815 | 5-cyclobutyl-3-{2-hydroxy-5-[(4-methylbenzoyl)amino]phenyl}-N-(4- |
| | methoxybenzyl)-1H-pyrazole-1-carboxamide |
| 1816 | N-(sec-butyl)-5-cyclobutyl-3-{2-hydroxy-5-[(4-methylbenzoyl)amino]phenyl}- |
| | 1H-pyrazole-1-carboxamide |
| 1817 | 5-cyclobutyl-N-(2-fluorobenzyl)-3-{2-hydroxy-5-[(4- |
| | methylbenzoyl)amino]phenyl}-1H-pyrazole-1-carboxamide |
| 1818 | 5-cyclobutyl-N-(3-fluorobenzyl)-3-{2-hydroxy-5-[(4- |
| | methylbenzoyl)amino]phenyl}-1H-pyrazole-1-carboxamide |
| 1819 | 5-cyclobutyl-N-cyclopropyl-3-{2-hydroxy-5-[(4-methylbenzoyl)amino]phenyl}- |
| | 1H-pyrazole-1-carboxamide |
| 1820 | 5-cyclopentyl-3-{2-hydroxy-5-[(4-methylbenzoyl)amino]phenyl}-N-isopropyl- |
| | 1H-pyrazole-1-carboxamide |
| 1821 | 5-cyclopentyl-N-ethyl-3-{2-hydroxy-5-[(4-methylbenzoyl)amino]phenyl}-1H- |
| | pyrazole-1-carboxamide |
| 1822 | N-allyl-5-cyclopentyl-3-{2-hydroxy-5-[(4-methylbenzoyl)amino]phenyl}-1H- |
| | pyrazole-1-carboxamide |
| 1823 | 5-cyclopentyl-3-{2-hydroxy-5-[(4-methylbenzoyl)amino]phenyl}-N-propyl-1H- |
| | pyrazole-1-carboxamide |
| 1824 | N-benzyl-5-cyclopentyl-3-{2-hydroxy-5-[(4-methylbenzoyl)amino]phenyl}-1H- |
| | pyrazole-1-carboxamide |
| 1825 | 5-cyclopentyl-3-{2-hydroxy-5-[(4-methylbenzoyl)amino]phenyl}-N-(2- |
| | phenylethyl)-1H-pyrazole-1-carboxamide |
| 1826 | 5-cyclopentyl-3-{2-hydroxy-5-[(4-methylbenzoyl)amino]phenyl}-N-(2- |
| | methylbenzyl)-1H-pyrazole-1-carboxamide |
| 1827 | 5-cyclopentyl-3-{2-hydroxy-5-[(4-methylbenzoyl)amino]phenyl}-N-(3- |
| | methylbenzyl)-1H-pyrazole-1-carboxamide |
| 1828 | 5-cyclopentyl-3-{2-hydroxy-5-[(4-methylbenzoyl)amino]phenyl}-N-(4- |
| | methylbenzyl)-1H-pyrazole-1-carboxamide |
| 1829 | N-(2-chlorobenzyl)-5-cyclopentyl-3-{2-hydroxy-5-[(4- |
| | methylbenzoyl)amino]phenyl}-1H-pyrazole-1-carboxamide |
| 1830 | 5-cyclopentyl-N-(4-fluorobenzyl)-3-{2-hydroxy-5-[(4- |
| | methylbenzoyl)amino]phenyl}-1H-pyrazole-1-carboxamide |
| 1831 | 5-cyclopentyl-N-(3,4-dichlorobenzyl)-3-{2-hydroxy-5-[(4- |
| | methylbenzoyl)amino]phenyl}-1H-pyrazole-1-carboxamide |
| 1832 | 5-cyclopentyl-3-{2-hydroxy-5-[(4-methylbenzoyl)amino]phenyl}-N-(4- |
| | methoxybenzyl)-1H-pyrazole-1-carboxamide |
| 1833 | N-(sec-butyl)-5-cyclopentyl-3-{2-hydroxy-5-[(4-methylbenzoyl)amino]phenyl}- |
| | 1H-pyrazole-1-carboxamide |
| 1834 | 5-cyclopentyl-N-(2-fluorobenzyl)-3-{2-hydroxy-5-[(4- |
| | methylbenzoyl)amino]phenyl}-1H-pyrazole-1-carboxamide |
| 1835 | 5-cyclopentyl-N-(3-fluorobenzyl)-3-{2-hydroxy-5-[(4- |
| | methylbenzoyl)amino]phenyl}-1H-pyrazole-1-carboxamide |
| 1836 | 5-cyclopentyl-N-cyclopropyl-3-{2-hydroxy-5-[(4-methylbenzoyl)amino]phenyl}- |
| | 1H-pyrazole-1-carboxamide |
| 1837 | 5-cyclopropyl-3-{5-[(4-ethylbenzoyl)amino]-2-hydroxyphenyl}-N-isopropyl-1H- |
| | pyrazole-1-carboxamide |
| 1838 | 5-cyclopropyl-N-ethyl-3-{5-[(4-ethylbenzoyl)amino]-2-hydroxyphenyl}-1H- |
| | pyrazole-1-carboxamide |
| 1839 | N-allyl-5-cyclopropyl-3-{5-[(4-ethylbenzoyl)amino]-2-hydroxyphenyl}-1H- |
| | pyrazole-1-carboxamide |
| 1840 | 5-cyclopropyl-3-{5-[(4-ethylbenzoyl)amino]-2-hydroxyphenyl}-N-propyl-1H- |
| | pyrazole-1-carboxamide |
| 1841 | N-benzyl-5-cyclopropyl-3-{5-[(4-ethylbenzoyl)amino]-2-hydroxyphenyl}-1H- |
| | pyrazole-1-carboxamide |
| 1842 | 5-cyclopropyl-3-{5-[(4-ethylbenzoyl)amino]-2-hydroxyphenyl}-N-(2- |
| | phenylethyl)-1H-pyrazole-1-carboxamide |
| 1843 | 5-cyclopropyl-3-{5-[(4-ethylbenzoyl)amino]-2-hydroxyphenyl}-N-(2- |
| | methylbenzyl)-1H-pyrazole-1-carboxamide |
| 1844 | 5-cyclopropyl-3-{5-[(4-ethylbenzoyl)amino]-2-hydroxyphenyl}-N-(3- |
| | methylbenzyl)-1H-pyrazole-1-carboxamide |
| 1845 | 5-cyclopropyl-3-{5-[(4-ethylbenzoyl)amino]-2-hydroxyphenyl}-N-(4- |
| | methylbenzyl)-1H-pyrazole-1-carboxamide |
| 1846 | N-(2-chlorobenzyl)-5-cyclopropyl-3-{5-[(4-ethylbenzoyl)amino]-2- |
| | hydroxyphenyl}-1H-pyrazole-1-carboxamide |
| 1847 | 5-cyclopropyl-3-{5-[(4-ethylbenzoyl)amino]-2-hydroxyphenyl}-N-(4- |
| | fluorobenzyl)-1H-pyrazole-1-carboxamide |
| 1848 | 5-cyclopropyl-N-(3,4-dichlorobenzyl)-3-{5-[(4-ethylbenzoyl)amino]-2- |
| | hydroxyphenyl}-1H-pyrazole-1-carboxamide |
| 1849 | 5-cyclopropyl-3-{5-[(4-ethylbenzoyl)amino]-2-hydroxyphenyl}-N-(4- |
| | methoxybenzyl)-1H-pyrazole-1-carboxamide |
| 1850 | N-(sec-butyl)-5-cyclopropyl-3-{5-[(4-ethylbenzoyl)amino]-2-hydroxyphenyl}-1H- |
| | pyrazole-1-carboxamide |
| 1851 | 5-cyclopropyl-3-{5-[(4-ethylbenzoyl)amino]-2-hydroxyphenyl}-N-(2- |
| | fluorobenzyl)-1H-pyrazole-1-carboxamide |
| 1852 | 5-cyclopropyl-3-{5-[(4-ethylbenzoyl)amino]-2-hydroxyphenyl}-N-(3- |
| | fluorobenzyl)-1H-pyrazole-1-carboxamide |
| 1853 | N,5-dicyclopropyl-3-{5-[(4-ethylbenzoyl)amino]-2-hydroxyphenyl}-1H-pyrazole- |
| | 1-carboxamide |
| 1854 | 5-cyclobutyl-3-{5-[(4-ethylbenzoyl)amino]-2-hydroxyphenyl}-N-isopropyl-1H- |
| | pyrazole-1-carboxamide |
| 1855 | 5-cyclobutyl-N-ethyl-3-{5-[(4-ethylbenzoyl)amino]-2-hydroxyphenyl}-1H- |
| | pyrazole-1-carboxamide |
| 1856 | N-allyl-5-cyclobutyl-3-{5-[(4-ethylbenzoyl)amino]-2-hydroxyphenyl}-1H- |
| | pyrazole-1-carboxamide |
| 1857 | 5-cyclobutyl-3-{5-[(4-ethylbenzoyl)amino]-2-hydroxyphenyl}-N-propyl-1H- |
| | pyrazole-1-carboxamide |
| 1858 | N-benzyl-5-cyclobutyl-3-{5-[(4-ethylbenzoyl)amino]-2-hydroxyphenyl}-1H- |
| | pyrazole-1-carboxamide |
| 1859 | 5-cyclobutyl-3-{5-[(4-ethylbenzoyl)amino]-2-hydroxyphenyl}-N-(2-phenylethyl)- |
| | 1H-pyrazole-1-carboxamide |
| 1860 | 5-cyclobutyl-3-{5-[(4-ethylbenzoyl)amino]-2-hydroxyphenyl}-N-(2- |
| | methylbenzyl)-1H-pyrazole-1-carboxamide |
| 1861 | 5-cyclobutyl-3-{5-[(4-ethylbenzoyl)amino]-2-hydroxyphenyl}-N-(3- |
| | methylbenzyl)-1H-pyrazole-1-carboxamide |
| 1862 | 5-cyclobutyl-3-{5-[(4-ethylbenzoyl)amino]-2-hydroxyphenyl}-N-(4- |
| | methylbenzyl)-1H-pyrazole-1-carboxamide |
| 1863 | N-(2-chlorobenzyl)-5-cyclobutyl-3-{5-[(4-ethylbenzoyl)amino]-2- |
| | hydroxyphenyl}-1H-pyrazole-1-carboxamide |
| 1864 | 5-cyclobutyl-3-{5-[(4-ethylbenzoyl)amino]-2-hydroxyphenyl}-N-(4- |
| | fluorobenzyl)-1H-pyrazole-1-carboxamide |
| 1865 | 5-cyclobutyl-N-(3,4-dichlorobenzyl)-3-{5-[(4-ethylbenzoyl)amino]-2- |
| | hydroxyphenyl}-1H-pyrazole-1-carboxamide |
| 1866 | 5-cyclobutyl-3-{5-[(4-ethylbenzoyl)amino]-2-hydroxyphenyl}-N-(4- |
| | methoxybenzyl)-1H-pyrazole-1-carboxamide |
| 1867 | N-(sec-butyl)-5-cyclobutyl-3-{5-[(4-ethylbenzoyl)amino]-2-hydroxyphenyl}-1H- |
| | pyrazole-1-carboxamide |
| 1868 | 5-cyclobutyl-3-{5-[(4-ethylbenzoyl)amino]-2-hydroxyphenyl}-N-(2- |
| | fluorobenzyl)-1H-pyrazole-1-carboxamide |
| 1869 | 5-cyclobutyl-3-{5-[(4-ethylbenzoyl)amino]-2-hydroxyphenyl}-N-(3- |
| | fluorobenzyl)-1H-pyrazole-1-carboxamide |
| 1870 | 5-cyclobutyl-N-cyclopropyl-3-{5-[(4-ethylbenzoyl)amino]-2-hydroxyphenyl}-1H- |
| | pyrazole-1-carboxamide |
| 1871 | 5-cyclopentyl-3-{5-[(4-ethylbenzoyl)amino]-2-hydroxyphenyl}-N-isopropyl-1H- |
| | pyrazole-1-carboxamide |
| 1872 | 5-cyclopentyl-N-ethyl-3-{5-[(4-ethylbenzoyl)amino]-2-hydroxyphenyl}-1H- |
| | pyrazole-1-carboxamide |
| 1873 | N-allyl-5-cyclopentyl-3-{5-[(4-ethylbenzoyl)amino]-2-hydroxyphenyl}-1H- |
| | pyrazole-1-carboxamide |
| 1874 | 5-cyclopentyl-3-{5-[(4-ethylbenzoyl)amino]-2-hydroxyphenyl}-N-propyl-1H- |
| | pyrazole-1-carboxamide |
| 1875 | N-benzyl-5-cyclopentyl-3-{5-[(4-ethylbenzoyl)amino]-2-hydroxyphenyl}-1H- |
| | pyrazole-1-carboxamide |
| 1876 | 5-cyclopentyl-3-{5-[(4-ethylbenzoyl)amino]-2-hydroxyphenyl}-N-(2- |
| | phenylethyl)-1H-pyrazole-1-carboxamide |
| 1877 | 5-cyclopentyl-3-{5-[(4-ethylbenzoyl)amino]-2-hydroxyphenyl}-N-(2- |
| | methylbenzyl)-1H-pyrazole-1-carboxamide |
| 1878 | 5-cyclopentyl-3-{5-[(4-ethylbenzoyl)amino]-2-hydroxyphenyl}-N-(3- |
| | methylbenzyl)-1H-pyrazole-1-carboxamide |
| 1879 | 5-cyclopentyl-3-{5-[(4-ethylbenzoyl)amino]-2-hydroxyphenyl}-N-(4- |
| | methylbenzyl)-1H-pyrazole-1-carboxamide |
| 1880 | N-(2-chlorobenzyl)-5-cyclopentyl-3-{5-[(4-ethylbenzoyl)amino]-2- |
| | hydroxyphenyl}-1H-pyrazole-1-carboxamide |
| 1881 | 5-cyclopentyl-3-{5-[(4-ethylbenzoyl)amino]-2-hydroxyphenyl}-N-(4- |
| | fluorobenzyl)-1H-pyrazole-1-carboxamide |
| 1882 | 5-cyclopentyl-N-(3,4-dichlorobenzyl)-3-{5-[(4-ethylbenzoyl)amino]-2- |
| | hydroxyphenyl}-1H-pyrazole-1-carboxamide |
| 1883 | 5-cyclopentyl-3-{5-[(4-ethylbenzoyl)amino]-2-hydroxyphenyl}-N-(4- |
| | methoxybenzyl)-1H-pyrazole-1-carboxamide |
| 1884 | N-(sec-butyl)-5-cyclopentyl-3-{5-[(4-ethylbenzoyl)amino]-2-hydroxyphenyl}-1H- |
| | pyrazole-1-carboxamide |
| 1885 | 5-cyclopentyl-3-{5-[(4-ethylbenzoyl)amino]-2-hydroxyphenyl}-N-(2- |
| | fluorobenzyl)-1H-pyrazole-1-carboxamide |
| 1886 | 5-cyclopentyl-3-{5-[(4-ethylbenzoyl)amino]-2-hydroxyphenyl}-N-(3- |
| | fluorobenzyl)-1H-pyrazole-1-carboxamide |
| 1887 | 5-cyclopentyl-N-cyclopropyl-3-{5-[(4-ethylbenzoyl)amino]-2-hydroxyphenyl}- |
| | 1H-pyrazole-1-carboxamide |
| 1888 | 5-cyclopropyl-3-[2-hydroxy-5-(isobutyrylamino)phenyl]-N-isopropyl-1H- |
| | pyrazole-1-carboxamide |
| 1889 | 5-cyclopropyl-N-ethyl-3-[2-hydroxy-5-(isobutyrylamino)phenyl]-1H-pyrazole-1- |
| | carboxamide |
| 1890 | N-allyl-5-cyclopropyl-3-[2-hydroxy-5-(isobutyrylamino)phenyl]-1H-pyrazole-1- |
| | carboxamide |
| 1891 | 5-cyclopropyl-3-[2-hydroxy-5-(isobutyrylamino)phenyl]-N-propyl-1H-pyrazole-1- |
| | carboxamide |
| 1892 | N-benzyl-5-cyclopropyl-3-[2-hydroxy-5-(isobutyrylamino)phenyl]-1H-pyrazole-1- |
| | carboxamide |
| 1893 | 5-cyclopropyl-3-[2-hydroxy-5-(isobutyrylamino)phenyl]-N-(2-phenylethyl)-1H- |
| | pyrazole-1-carboxamide |
| 1894 | 5-cyclopropyl-3-[2-hydroxy-5-(isobutyrylamino)phenyl]-N-(2-methylbenzyl)-1H- |
| | pyrazole-1-carboxamide |
| 1895 | 5-cyclopropyl-3-[2-hydroxy-5-(isobutyrylamino)phenyl]-N-(3-methylbenzyl)-1H- |
| | pyrazole-1-carboxamide |
| 1896 | 5-cyclopropyl-3-[2-hydroxy-5-(isobutyrylamino)phenyl]-N-(4-methylbenzyl)-1H- |
| | pyrazole-1-carboxamide |
| 1897 | N-(2-chlorobenzyl)-5-cyclopropyl-3-[2-hydroxy-5-(isobutyrylamino)phenyl]-1H- |
| | pyrazole-1-carboxamide |
| 1898 | 5-cyclopropyl-N-(4-fluorobenzyl)-3-[2-hydroxy-5-(isobutyrylamino)phenyl]-1H- |
| | pyrazole-1-carboxamide |
| 1899 | 5-cyclopropyl-N-(3,4-dichlorobenzyl)-3-[2-hydroxy-5-(isobutyrylamino)phenyl]- |
| | 1H-pyrazole-1-carboxamide |
| 1900 | 5-cyclopropyl-3-[2-hydroxy-5-(isobutyrylamino)phenyl]-N-(4-methoxybenzyl)- |
| | 1H-pyrazole-1-carboxamide |
| 1901 | N-(sec-butyl)-5-cyclopropyl-3-[2-hydroxy-5-(isobutyrylamino)phenyl]-1H- |
| | pyrazole-1-carboxamide |
| 1902 | 5-cyclopropyl-N-(2-fluorobenzyl)-3-[2-hydroxy-5-(isobutyrylamino)phenyl]-1H- |
| | pyrazole-1-carboxamide |
| 1903 | 5-cyclopropyl-N-(3-fluorobenzyl)-3-[2-hydroxy-5-(isobutyrylamino)phenyl]-1H- |
| | pyrazole-1-carboxamide |
| 1904 | N,5-dicyclopropyl-3-[2-hydroxy-5-(isobutyrylamino)phenyl]-1H-pyrazole-1- |
| | carboxamide |
| 1905 | 5-cyclobutyl-3-[2-hydroxy-5-(isobutyrylamino)phenyl]-N-isopropyl-1H-pyrazole- |
| | 1-carboxamide |
| 1906 | 5-cyclobutyl-N-ethyl-3-[2-hydroxy-5-(isobutyrylamino)phenyl]-1H-pyrazole-1- |
| | carboxamide |
| 1907 | N-allyl-5-cyclobutyl-3-[2-hydroxy-5-(isobutyrylamino)phenyl]-1H-pyrazole-1- |
| | carboxamide |
| 1908 | 5-cyclobutyl-3-[2-hydroxy-5-(isobutyrylamino)phenyl]-N-propyl-1H-pyrazole-1- |
| | carboxamide |
| 1909 | N-benzyl-5-cyclobutyl-3-[2-hydroxy-5-(isobutyrylamino)phenyl]-1H-pyrazole-1- |
| | carboxamide |
| 1910 | 5-cyclobutyl-3-[2-hydroxy-5-(isobutyrylamino)phenyl]-N-(2-phenylethyl)-1H- |
| | pyrazole-1-carboxamide |
| 1911 | 5-cyclobutyl-3-[2-hydroxy-5-(isobutyrylamino)phenyl]-N-(2-methylbenzyl)-1H- |
| | pyrazole-1-carboxamide |
| 1912 | 5-cyclobutyl-3-[2-hydroxy-5-(isobutyrylamino)phenyl]-N-(3-methylbenzyl)-1H- |
| | pyrazole-1-carboxamide |
| 1913 | 5-cyclobutyl-3-[2-hydroxy-5-(isobutyrylamino)phenyl]-N-(4-methylbenzyl)-1H- |
| | pyrazole-1-carboxamide |
| 1914 | N-(2-chlorobenzyl)-5-cyclobutyl-3-[2-hydroxy-5-(isobutyrylamino)phenyl]-1H- |
| | pyrazole-1-carboxamide |
| 1915 | 5-cyclobutyl-N-(4-fluorobenzyl)-3-[2-hydroxy-5-(isobutyrylamino)phenyl]-1H- |
| | pyrazole-1-carboxamide |
| 1916 | 5-cyclobutyl-N-(3,4-dichlorobenzyl)-3-[2-hydroxy-5-(isobutyrylamino)phenyl]- |
| | 1H-pyrazole-1-carboxamide |
| 1917 | 5-cyclobutyl-3-[2-hydroxy-5-(isobutyrylamino)phenyl]-N-(4-methoxybenzyl)-1H- |
| | pyrazole-1-carboxamide |
| 1918 | N-(sec-butyl)-5-cyclobutyl-3-[2-hydroxy-5-(isobutyrylamino)phenyl]-1H- |
| | pyrazole-1-carboxamide |
| 1919 | 5-cyclobutyl-N-(2-fluorobenzyl)-3-[2-hydroxy-5-(isobutyrylamino)phenyl]-1H- |
| | pyrazole-1-carboxamide |
| 1920 | 5-cyclobutyl-N-(3-fluorobenzyl)-3-[2-hydroxy-5-(isobutyrylamino)phenyl]-1H- |
| | pyrazole-1-carboxamide |
| 1921 | 5-cyclobutyl-N-cyclopropyl-3-[2-hydroxy-5-(isobutyrylamino)phenyl]-1H- |
| | pyrazole-1-carboxamide |
| 1922 | 5-cyclopentyl-3-[2-hydroxy-5-(isobutyrylamino)phenyl]-N-isopropyl-1H- |
| | pyrazole-1-carboxamide |
| 1923 | 5-cyclopentyl-N-ethyl-3-[2-hydroxy-5-(isobutyrylamino)phenyl]-1H-pyrazole-1- |
| | carboxamide |
| 1924 | N-allyl-5-cyclopentyl-3-[2-hydroxy-5-(isobutyrylamino)phenyl]-1H-pyrazole-1- |
| | carboxamide |
| 1925 | 5-cyclopentyl-3-[2-hydroxy-5-(isobutyrylamino)phenyl]-N-propyl-1H-pyrazole-1- |
| | carboxamide |
| 1926 | N-benzyl-5-cyclopentyl-3-[2-hydroxy-5-(isobutyrylamino)phenyl]-1H-pyrazole-1- |
| | carboxamide |
| 1927 | 5-cyclopentyl-3-[2-hydroxy-5-(isobutyrylamino)phenyl]-N-(2-phenylethyl)-1H- |
| | pyrazole-1-carboxamide |
| 1928 | 5-cyclopentyl-3-[2-hydroxy-5-(isobutyrylamino)phenyl]-N-(2-methylbenzyl)-1H- |
| | pyrazole-1-carboxamide |
| 1929 | 5-cyclopentyl-3-[2-hydroxy-5-(isobutyrylamino)phenyl]-N-(3-methylbenzyl)-1H- |
| | pyrazole-1-carboxamide |
| 1930 | 5-cyclopentyl-3-[2-hydroxy-5-(isobutyrylamino)phenyl]-N-(4-methylbenzyl)-1H- |
| | pyrazole-1-carboxamide |
| 1931 | N-(2-chlorobenzyl)-5-cyclopentyl-3-[2-hydroxy-5-(isobutyrylamino)phenyl]-1H- |
| | pyrazole-1-carboxamide |
| 1932 | 5-cyclopentyl-N-(4-fluorobenzyl)-3-[2-hydroxy-5-(isobutyrylamino)phenyl]-1H- |
| | pyrazole-1-carboxamide |
| 1933 | 5-cyclopentyl-N-(3,4-dichlorobenzyl)-3-[2-hydroxy-5-(isobutyrylamino)phenyl]- |
| | 1H-pyrazole-1-carboxamide |
| 1934 | 5-cyclopentyl-3-[2-hydroxy-5-(isobutyrylamino)phenyl]-N-(4-methoxybenzyl)- |
| | 1H-pyrazole-1-carboxamide |
| 1935 | N-(sec-butyl)-5-cyclopentyl-3-[2-hydroxy-5-(isobutyrylamino)phenyl]-1H- |
| | pyrazole-1-carboxamide |
| 1936 | 5-cyclopentyl-N-(2-fluorobenzyl)-3-[2-hydroxy-5-(isobutyrylamino)phenyl]-1H- |
| | pyrazole-1-carboxamide |
| 1937 | 5-cyclopentyl-N-(3-fluorobenzyl)-3-[2-hydroxy-5-(isobutyrylamino)phenyl]-1H- |
| | pyrazole-1-carboxamide |
| 1938 | 5-cyclopentyl-N-cyclopropyl-3-[2-hydroxy-5-(isobutyrylamino)phenyl]-1H- |
| | pyrazole-1-carboxamide |
| 1939 | 3-[5-(acetylamino)-2-hydroxyphenyl]-5-cyclopropyl-N-isopropyl-1H-pyrazole-1- |
| | carboxamide |
| 1940 | 3-[5-(acetylamino)-2-hydroxyphenyl]-5-cyclopropyl-N-ethyl-1H-pyrazole-1- |
| | carboxamide |
| 1941 | 3-[5-(acetylamino)-2-hydroxyphenyl]-N-allyl-5-cyclopropyl-1H-pyrazole-1- |
| | carboxamide |
| 1942 | 3-[5-(acetylamino)-2-hydroxyphenyl]-5-cyclopropyl-N-propyl-1H-pyrazole-1- |
| | carboxamide |
| 1943 | 3-[5-(acetylamino)-2-hydroxyphenyl]-N-benzyl-5-cyclopropyl-1H-pyrazole-1- |
| | carboxamide |
| 1944 | 3-[5-(acetylamino)-2-hydroxyphenyl]-5-cyclopropyl-N-(2-phenylethyl)-1H- |
| | pyrazole-1-carboxamide |
| 1945 | 3-[5-(acetylamino)-2-hydroxyphenyl]5-cyclopropyl-N-(2-methylbenzyl)-1H- |
| | pyrazole-1-carboxamide |
| 1946 | 3-[5-(acetylamino)-2-hydroxyphenyl]-5-cyclopropyl-N-(3-methylbenzyl)-1H- |
| | pyrazole-1-carboxamide |
| 1947 | 3-[5-(acetylamino)-2-hydroxyphenyl]-5-cyclopropyl-N-(4-methylbenzyl)-1H- |
| | pyrazole-1-carboxamide |
| 1948 | 3-[5-(acetylamino)-2-hydroxyphenyl]-N-(2-chlorobenzyl)-5-cyclopropyl-1H- |
| | pyrazole-1-carboxamide |
| 1949 | 3-[5-(acetylamino)-2-hydroxyphenyl]-5-cyclopropyl-N-(4-fluorobenzyl)-1H- |
| | pyrazole-1-carboxamide |
| 1950 | 3-[5-(acetylamino)-2-hydroxyphenyl]-5-cyclopropyl-N-(3,4-dichlorobenzyl)-1H- |
| | pyrazole-1-carboxamide |
| 1951 | 3-[5-(acetylamino)-2-hydroxyphenyl]-5-cyclopropyl-N-(4-methoxybenzyl)-1H- |
| | pyrazole-1-carboxamide |
| 1952 | 3-[5-(acetylamino)-2-hydroxyphenyl]-N-(sec-butyl)-5-cyclopropyl-1H-pyrazole-1- |
| | carboxamide |
| 1953 | 3-[5-(acetylamino)-2-hydroxyphenyl]-5-cyclopropyl-N-(2-fluorobenzyl)-1H- |
| | pyrazole-1-carboxamide |
| 1954 | 3-[5-(acetylamino)-2-hydroxyphenyl]-5-cyclopropyl-N-(3-fluorobenzyl)-1H- |
| | pyrazole-1-carboxamide |
| 1955 | 3-[5-(acetylamino)-2-hydroxyphenyl]-N,5-dicyclopropyl-1H-pyrazole-1- |
| | carboxamide |
| 1956 | 3-[5-(acetylamino)-2-hydroxyphenyl]-5-cyclobutyl-N-isopropyl-1H-pyrazole-1- |
| | carboxamide |
| 1957 | 3-[5-(acetylamino)-2-hydroxyphenyl]-5-cyclobutyl-N-ethyl-1H-pyrazole-1- |
| | carboxamide |
| 1958 | 3-[5-(acetylamino)-2-hydroxyphenyl]-N-allyl-5-cyclobutyl-1H-pyrazole-1- |
| | carboxamide |
| 1959 | 3-[5-(acetylamino)-2-hydroxyphenyl]-5-cyclobutyl-N-propyl-1H-pyrazole-1- |
| | carboxamide |
| 1960 | 3-[5-(acetylamino)-2-hydroxyphenyl]-N-benzyl-5-cyclobutyl-1H-pyrazole-1- |
| | carboxamide |
| 1961 | 3-[5-(acetylamino)-2-hydroxyphenyl]-5-cyclobutyl-N-(2-phenylethyl)-1H- |
| | pyrazole-1-carboxamide |
| 1962 | 3-[5-(acetylamino)-2-hydroxyphenyl]-5-cyclobutyl-N-(2-methylbenzyl)-1H- |
| | pyrazole-1-carboxamide |
| 1963 | 3-[5-(acetylamino)-2-hydroxyphenyl]-5-cyclobutyl-N-(3-methylbenzyl)-1H- |
| | pyrazole-1-carboxamide |
| 1964 | 3-[5-(acetylamino)-2-hydroxyphenyl]-5-cyclobutyl-N-(4-methylbenzyl)-1H- |
| | pyrazole-1-carboxamide |
| 1965 | 3-[5-(acetylamino)-2-hydroxyphenyl]-N-(2-chlorobenzyl)-5-cyclobutyl-1H- |
| | pyrazole-1-carboxamide |
| 1966 | 3-[5-(acetylamino)-2-hydroxyphenyl]-5-cyclobutyl-N-(4-fluorobenzyl)-1H- |
| | pyrazole-1-carboxamide |
| 1967 | 3-[5-(acetylamino)-2-hydroxyphenyl]-5-cyclobutyl-N-(3,4-dichlorobenzyl)-1H- |
| | pyrazole-1-carboxamide |
| 1968 | 3-[5-(acetylamino)-2-hydroxyphenyl]-5-cyclobutyl-N-(4-methoxybenzyl)-1H- |
| | pyrazole-1-carboxamide |
| 1969 | 3-[5-(acetylamino)-2-hydroxyphenyl]-N-(sec-butyl)-5-cyclobutyl-1H-pyrazole-1- |
| | carboxamide |
| 1970 | 3-[5-(acetylamino)-2-hydroxyphenyl]-5-cyclobutyl-N-(2-fluorobenzyl)-1H- |
| | pyrazole-1-carboxamide |
| 1971 | 3-[5-(acetylamino)-2-hydroxyphenyl]-5-cyclobutyl-N-(3-fluorobenzyl)-1H- |
| | pyrazole-1-carboxamide |
| 1972 | 3-[5-(acetylamino)-2-hydroxyphenyl]-5-cyclobutyl-N-cyclopropyl-1H-pyrazole-1- |
| | carboxamide |
| 1973 | 3-[5-(acetylamino)-2-hydroxyphenyl]-5-cyclopentyl-N-isopropyl-1H-pyrazole-1- |
| | carboxamide |
| 1974 | 3-[5-(acetylamino)-2-hydroxyphenyl]-5-cyclopentyl-N-ethyl-1H-pyrazole-1- |
| | carboxamide |
| 1975 | 3-[5-(acetylamino)-2-hydroxyphenyl]-N-allyl-5-cyclopentyl-1H-pyrazole-1- |
| | carboxamide |
| 1976 | 3-[5-(acetylamino)-2-hydroxyphenyl]-5-cyclopentyl-N-propyl-1H-pyrazole-1- |
| | carboxamide |
| 1977 | 3-[5-(acetylamino)-2-hydroxyphenyl]-N-benzyl-5-cyclopentyl-1H-pyrazole-1- |
| | carboxamide |
| 1978 | 3-[5-(acetylamino)-2-hydroxyphenyl]-5-cyclopentyl-N-(2-phenylethyl)-1H- |
| | pyrazole-1-carboxamide |
| 1979 | 3-[5-(acetylamino)-2-hydroxyphenyl]-5-cyclopentyl-N-(2-methylbenzyl)-1H- |
| | pyrazole-1-carboxamide |
| 1980 | 3-[5-(acetylamino)-2-hydroxyphenyl]-5-cyclopentyl-N-(3-methylbenzyl)-1H- |
| | pyrazole-1-carboxamide |
| 1981 | 3-[5-(acetylamino)-2-hydroxyphenyl]-5-cyclopentyl-N-(4-methylbenzyl)-1H- |
| | pyrazole-1-carboxamide |
| 1982 | 3-[5-(acetylamino)-2-hydroxyphenyl]-N-(2-chlorobenzyl)-5-cyclopentyl-1H- |
| | pyrazole-1-carboxamide |
| 1983 | 3-[5-(acetylamino)-2-hydroxyphenyl]-5-cyclopentyl-N-(4-fluorobenzyl)-1H- |
| | pyrazole-1-carboxamide |
| 1984 | 3-[5-(acetylamino)-2-hydroxyphenyl]-5-cyclopentyl-N-(3,4-dichlorobenzyl)-1H- |
| | pyrazole-1-carboxamide |
| 1985 | 3-[5-(acetylamino)-2-hydroxyphenyl]-5-cyclopentyl-N-(4-methoxybenzyl)-1H- |
| | pyrazole-1-carboxamide |
| 1986 | 3-[5-(acetylamino)-2-hydroxyphenyl]-N-(sec-butyl)-5-cyclopentyl-1H-pyrazole-1- |
| | carboxamide |
| 1987 | 3-[5-(acetylamino)-2-hydroxyphenyl]-5-cyclopentyl-N-(2-fluorobenzyl)-1H- |
| | pyrazole-1-carboxamide |
| 1988 | 3-[5-(acetylamino)-2-hydroxyphenyl]-5-cyclopentyl-N-(3-fluorobenzyl)-1H- |
| | pyrazole-1-carboxamide |
| 1989 | 3-[5-(acetylamino)-2-hydroxyphenyl]-5-cyclopentyl-N-cyclopropyl-1H-pyrazole- |
| | 1-carboxamide |
| 1990 | 5-cyclopropyl-3-{2-hydroxy-5-[(3-methylbutanoyl)amino]phenyl}-N-isopropyl- |
| | 1H-pyrazole-1-carboxamide |
| 1991 | 5-cyclopropyl-N-ethyl-3-{2-hydroxy-5-[(3-methylbutanoyl)amino]phenyl}-1H- |
| | pyrazole-1-carboxamide |
| 1992 | N-allyl-5-cyclopropyl-3-{2-hydroxy-5-[(3-methylbutanoyl)amino]phenyl}-1H- |
| | pyrazole-1-carboxamide |
| 1993 | 5-cyclopropyl-3-{2-hydroxy-5-[(3-methylbutanoyl)amino]phenyl}-N-propyl-1H- |
| | pyrazole-1-carboxamide |
| 1994 | N-benzyl-5-cyclopropyl-3-{2-hydroxy-5-[(3-methylbutanoyl)amino]phenyl}-1H- |
| | pyrazole-1-carboxamide |
| 1995 | 5-cyclopropyl-3-{2-hydroxy-5-[(3-methylbutanoyl)amino]phenyl}-N-(2- |
| | phenylethyl)-1H-pyrazole-1-carboxamide |
| 1996 | 5-cyclopropyl-3-{2-hydroxy-5-[(3-methylbutanoyl)amino]phenyl}-N-(2- |
| | methylbenzyl)-1H-pyrazole-1-carboxamide |
| 1997 | 5-cyclopropyl-3-{2-hydroxy-5-[(3-methylbutanoyl)amino]phenyl}-N-(3- |
| | methylbenzyl)-1H-pyrazole-1-carboxamide |
| 1998 | 5-cyclopropyl-3-{2-hydroxy-5-[(3-methylbutanoyl)amino]phenyl}-N-(4- |
| | methylbenzyl)-1H-pyrazole-1-carboxamide |
| 1999 | N-(2-chlorobenzyl)-5-cyclopropyl-3-{2-hydroxy-5-[(3- |
| | methylbutanoyl)amino]phenyl}-1H-pyrazole-1-carboxamide |
| 2000 | 5-cyclopropyl-N-(4-fluorobenzyl)-3-{2-hydroxy-5-[(3- |
| | methylbutanoyl)amino]phenyl}-1H-pyrazole-1-carboxamide |
| 2001 | 5-cyclopropyl-N-(3,4-dichlorobenzyl)-3-{2-hydroxy-5-[(3- |
| | methylbutanoyl)amino]phenyl}-1H-pyrazole-1-carboxamide |
| 2002 | 5-cyclopropyl-3-{2-hydroxy-5-[(3-methylbutanoyl)amino]phenyl}-N-(4- |
| | methoxybenzyl)-1H-pyrazole-1-carboxamide |
| 2003 | N-(sec-butyl)-5-cyclopropyl-3-{2-hydroxy-5-[(3-methylbutanoyl)amino]phenyl}- |
| | 1H-pyrazole-1-carboxamide |
| 2004 | 5-cyclopropyl-N-(2-fluorobenzyl)-3-{2-hydroxy-5-[(3- |
| | methylbutanoyl)amino]phenyl}-1H-pyrazole-1-carboxamide |
| 2005 | 5-cyclopropyl-N-(3-fluorobenzyl)-3-{2-hydroxy-5-[(3- |
| | methylbutanoyl)amino]phenyl}-1H-pyrazole-1-carboxamide |
| 2006 | N,5-dicyclopropyl-3-{2-hydroxy-5-[(3-methylbutanoyl)amino]phenyl}-1H- |
| | pyrazole-1-carboxamide |
| 2007 | 5-cyclobutyl-3-{2-hydroxy-5-[(3-methylbutanoyl)amino]phenyl}-N-isopropyl-1H- |
| | pyrazole-1-carboxamide |
| 2008 | 5-cyclobutyl-N-ethyl-3-{2-hydroxy-5-[(3-methylbutanoyl)amino]phenyl}-1H- |
| | pyrazole-1-carboxamide |
| 2009 | N-allyl-5-cyclobutyl-3-{2-hydroxy-5-[(3-methylbutanoyl)amino]phenyl}-1H- |
| | pyrazole-1-carboxamide |
| 2010 | 5-cyclobutyl-3-{2-hydroxy-5-[(3-methylbutanoyl)amino]phenyl}-N-propyl-1H- |
| | pyrazole-1-carboxamide |
| 2011 | N-benzyl-5-cyclobutyl-3-{2-hydroxy-5-[(3-methylbutanoyl)amino]phenyl}-1H- |
| | pyrazole-1-carboxamide |
| 2012 | 5-cyclobutyl-3-{2-hydroxy-5-[(3-methylbutanoyl)amino]phenyl}-N-(2- |
| | phenylethyl)-1H-pyrazole-1-carboxamide |
| 2013 | 5-cyclobutyl-3-{2-hydroxy-5-[(3-methylbutanoyl)amino]phenyl}-N-(2- |
| | methylbenzyl)-1H-pyrazole-1-carboxamide |
| 2014 | 5-cyclobutyl-3-{2-hydroxy-5-[(3-methylbutanoyl)amino]phenyl}-N-(3- |
| | methylbenzyl)-1H-pyrazole-1-carboxamide |
| 2015 | 5-cyclobutyl-3-{2-hydroxy-5-[(3-methylbutanoyl)amino]phenyl}-N-(4- |
| | methylbenzyl)-1H-pyrazole-1-carboxamide |
| 2016 | N-(2-chlorobenzyl)-5-cyclobutyl-3-{2-hydroxy-5-[(3- |
| | methylbutanoyl)amino]phenyl}-1H-pyrazole-1-carboxamide |
| 2017 | 5-cyclobutyl-N-(4-fluorobenzyl)-3-{2-hydroxy-5-[(3- |
| | methylbutanoyl)amino]phenyl}-1H-pyrazole-1-carboxamide |
| 2018 | 5-cyclobutyl-N-(3,4-dichlorobenzyl)-3-{2-hydroxy-5-[(3- |
| | methylbutanoyl)amino]phenyl}-1H-pyrazole-1-carboxamide |
| 2019 | 5-cyclobutyl-3-{2-hydroxy-5-[(3-methylbutanoyl)amino]phenyl}-N-(4- |
| | methoxybenzyl)-1H-pyrazole-1-carboxamide |
| 2020 | N-(sec-butyl)-5-cyclobutyl-3-{2-hydroxy-5-[(3-methylbutanoyl)amino]phenyl}- |
| | 1H-pyrazole-1-carboxamide |
| 2021 | 5-cyclobutyl-N-(2-fluorobenzyl)-3-{2-hydroxy-5-[(3- |
| | methylbutanoyl)amino]phenyl}-1H-pyrazole-1-carboxamide |
| 2022 | 5-cyclobutyl-N-(3-fluorobenzyl)-3-{2-hydroxy-5-[(3- |
| | methylbutanoyl)amino]phenyl}-1H-pyrazole-1-carboxamide |
| 2023 | 5-cyclobutyl-N-cyclopropyl-3-{2-hydroxy-5-[(3-methylbutanoyl)amino]phenyl}- |
| | 1H-pyrazole-1-carboxamide |
| 2024 | 5-cyclopentyl-3-{2-hydroxy-5-[(3-methylbutanoyl)amino]phenyl}-N-isopropyl- |
| | 1H-pyrazole-1-carboxamide |
| 2025 | 5-cyclopentyl-N-ethyl-3-{2-hydroxy-5-[(3-methylbutanoyl)amino]phenyl}-1H- |
| | pyrazole-1-carboxamide |
| 2026 | N-allyl-5-cyclopentyl-3-{2-hydroxy-5-[(3-methylbutanoyl)amino]phenyl}-1H- |
| | pyrazole-1-carboxamide |
| 2027 | 5-cyclopentyl-3-{2-hydroxy-5-[(3-methylbutanoyl)amino]phenyl}-N-propyl-1H- |
| | pyrazole-1-carboxamide |
| 2028 | N-benzyl-5-cyclopentyl-3-{2-hydroxy-5-[(3-methylbutanoyl)amino]phenyl}-1H- |
| | pyrazole-1-carboxamide |
| 2029 | 5-cyclopentyl-3-{2-hydroxy-5-[(3-methylbutanoyl)amino]phenyl}-N-(2- |
| | phenylethyl)-1H-pyrazole-1-carboxamide |
| 2030 | 5-cyclopentyl-3-{2-hydroxy-5-[(3-methylbutanoyl)amino]phenyl}-N-(2- |
| | methylbenzyl)-1H-pyrazole-1-carboxamide |
| 2031 | 5-cyclopentyl-3-{2-hydroxy-5-[(3-methylbutanoyl)amino]phenyl}-N-(3- |
| | methylbenzyl)-1H-pyrazole-1-carboxamide |
| 2032 | 5-cyclopentyl-3-{2-hydroxy-5-[(3-methylbutanoyl)amino]phenyl}-N-(4- |
| | methylbenzyl)-1H-pyrazole-1-carboxamide |
| 2033 | N-(2-chlorobenzyl)-5-cyclopentyl-3-{2-hydroxy-5-[(3- |
| | methylbutanoyl)amino]phenyl}-1H-pyrazole-1-carboxamide |
| 2034 | 5-cyclopentyl-N-(4-fluorobenzyl)-3-{2-hydroxy-5-[(3- |
| | methylbutanoyl)amino]phenyl}-1H-pyrazole-1-carboxamide |
| 2035 | 5-cyclopentyl-N-(3,4-dichlorobenzyl)-3-{2-hydroxy-5-[(3- |
| | methylbutanoyl)amino]phenyl}-1H-pyrazole-1-carboxamide |
| 2036 | 5-cyclopentyl-3-{2-hydroxy-5-[(3-methylbutanoyl)amino]phenyl}-N-(4- |
| | methoxybenzyl)-1H-pyrazole-1-carboxamide |
| 2037 | N-(sec-butyl)-5-cyclopentyl-3-{2-hydroxy-5-[(3-methylbutanoyl)amino]phenyl}- |
| | 1H-pyrazole-1-carboxamide |
| 2038 | 5-cyclopentyl-N-(2-fluorobenzyl)-3-{2-hydroxy-5-[(3- |
| | methylbutanoyl)amino]phenyl}-1H-pyrazole-1-carboxamide |
| 2039 | 5-cyclopentyl-N-(3-fluorobenzyl)-3-{2-hydroxy-5-[(3- |
| | methylbutanoyl)amino]phenyl}-1H-pyrazole-1-carboxamide |
| 2040 | 5-cyclopentyl-N-cyclopropyl-3-{2-hydroxy-5-[(3-methylbutanoyl)amino]phenyl}- |
| | 1H-pyrazole-1-carboxamide |
| 2041 | 5-cyclopropyl-3-[2-hydroxy-5-(propionylamino)phenyl]-N-isopropyl-1H- |
| | pyrazole-1-carboxamide |
| 2042 | 5-cyclopropyl-N-ethyl-3-[2-hydroxy-5-(propionylamino)phenyl]-1H-pyrazole-1- |
| | carboxamide |
| 2043 | N-allyl-5-cyclopropyl-3-[2-hydroxy-5-(propionylamino)phenyl]-1H-pyrazole-1- |
| | carboxamide |
| 2044 | 5-cyclopropyl-3-[2-hydroxy-5-(propionylamino)phenyl]-N-propyl-1H-pyrazole-1- |
| | carboxamide |
| 2045 | N-benzyl-5-cyclopropyl-3-[2-hydroxy-5-(propionylamino)phenyl]-1H-pyrazole-1- |
| | carboxamide |
| 2046 | 5-cyclopropyl-3-[2-hydroxy-5-(propionylamino)phenyl]-N-(2-phenylethyl)-1H- |
| | pyrazole-1-carboxamide |
| 2047 | 5-cyclopropyl-3-[2-hydroxy-5-(propionylamino)phenyl]-N-(2-methylbenzyl)-1H- |
| | pyrazole-1-carboxamide |
| 2048 | 5-cyclopropyl-3-[2-hydroxy-5-(propionylamino)phenyl]-N-(3-methylbenzyl)-1H- |
| | pyrazole-1-carboxamide |
| 2049 | 5-cyclopropyl-3-[2-hydroxy-5-(propionylamino)phenyl]-N-(4-methylbenzyly)-1H- |
| | pyrazole-1-carboxamide |
| 2050 | N-(2-chlorobenzyl)-5-cyclopropyl-3-[2-hydroxy-5-(propionylamino)phenyl]-1H- |
| | pyrazole-1-carboxamide |
| 2051 | 5-cyclopropyl-N-(4-fluorobenzyl)-3-[2-hydroxy-5-(propionylamino)phenyl]-1H- |
| | pyrazole-1-carboxamide |
| 2052 | 5-cyclopropyl-N-(3,4-dichlorobenzyl)-3-[2-hydroxy-5-(propionylamino)phenyl]- |
| | 1H-pyrazole-1-carboxamide |
| 2053 | 5-cyclopropyl-3-[2-hydroxy-5-(propionylamino)phenyl]-N-(4-methoxybenzyl)- |
| | 1H-pyrazole-1-carboxamide |
| 2054 | N-(sec-butyl)-5-cyclopropyl-3-[2-hydroxy-5-(propionylamino)phenyl]-1H- |
| | pyrazole-1-carboxamide |
| 2055 | 5-cyclopropyl-N-(2-fluorobenzyl)-3-[2-hydroxy-5-(propionylamino)phenyl]-1H- |
| | pyrazole-1-carboxamide |
| 2056 | 5-cyclopropyl-N-(3-fluorobenzyl)-3-[2-hydroxy-5-(propionylamino)phenyl]-1H- |
| | pyrazole-1-carboxamide |
| 2057 | N,5-dicyclopropyl-3-[2-hydroxy-5-(propionylamino)phenyl]-1H-pyrazole-1- |
| | carboxamide |
| 2058 | 5-cyclobutyl-3-[2-hydroxy-5-(propionylamino)phenyl]-N-isopropyl-1H-pyrazole- |
| | 1-carboxamide |
| 2059 | 5-cyclobutyl-N-ethyl-3-[2-hydroxy-5-(propionylamino)phenyl]-1H-pyrazole-1- |
| | carboxamide |
| 2060 | N-allyl-5-cyclobutyl-3-[2-hydroxy-5-(propionylamino)phenyl]-1H-pyrazole-1- |
| | carboxamide |
| 2061 | 5-cyclobutyl-3-[2-hydroxy-5-(propionylamino)phenyl]-N-propyl-1H-pyrazole-1- |
| | carboxamide |
| 2062 | N-benzyl-5-cyclobutyl-3-[2-hydroxy-5-(propionylamino)phenyl]-1H-pyrazole-1- |
| | carboxamide |
| 2063 | 5-cyclobutyl-3-[2-hydroxy-5-(propionylamino)phenyl]-N-(2-phenylethyl)-1H- |
| | pyrazole-1-carboxamide |
| 2064 | 5-cyclobutyl-3-[2-hydroxy-5-(propionylamino)phenyl]-N-(2-methylbenzyl)-1H- |
| | pyrazole-1-carboxamide |
| 2065 | 5-cyclobutyl-3-[2-hydroxy-5-(propionylamino)phenyl]-N-(3-methylbenzyl)-1H- |
| | pyrazole-1-carboxamide |
| 2066 | 5-cyclobutyl-3-[2-hydroxy-5-(propionylamino)phenyl]-N-(4-methylbenzyl)-1H- |
| | pyrazole-1-carboxamide |
| 2067 | N-(2-chlorobenzyl)-5-cyclobutyl-3-[2-hydroxy-5-(propionylamino)phenyl]-1H- |
| | pyrazole-1-carboxamide |
| 2068 | 5-cyclobutyl-N-(4-fluorobenzyl)-3-[2-hydroxy-5-{propionylamino)phenyl]-1H- |
| | pyrazole-1-carboxamide |
| 2069 | 5-cyclobutyl-N-(3,4-dichlorobenzyl)-3-[2-hydroxy-5-(propionylamino)phenyl]- |
| | 1H-pyrazole-1-carboxamide |
| 2070 | 5-cyclobutyl-3-[2-hydroxy-5-(propionylamino)phenyl]-N-(4-methoxybenzyl)-1H- |
| | pyrazole-1-carboxamide |
| 2071 | N-(sec-butyl)-5-cyclobutyl-3-[2-hydroxy-5-(propionylamino)phenyl]-1H- |
| | pyrazole-1-carboxamide |
| 2072 | 5-cyclobutyl-N-(2-fluorobenzyl)-3-[2-hydroxy-5-(propionylamino)phenyl]-1H- |
| | pyrazole-1-carboxamide |
| 2073 | 5-cyclobutyl-N-(3-fluorobenzyl)-3-[2-hydroxy-5-(propionylamino)phenyl]-1H- |
| | pyrazole-1-carboxamide |
| 2074 | 5-cyclobutyl-N-cyclopropyl-3-[2-hydroxy-5-(propionylamino)phenyl]-1H- |
| | pyrazole-1-carboxamide |
| 2075 | 5-cyclopentyl-3-[2-hydroxy-5-(propionylamino)phenyl]-N-isopropyl-1H-pyrazole- |
| | 1-carboxamide |
| 2076 | 5-cyclopentyl-N-ethyl-3-[2-hydroxy-5-(propionylamino)phenyl]-1H-pyrazole-1- |
| | carboxamide |
| 2077 | N-allyl-5-cyclopentyl-3-[2-hydroxy-5-(propionylamino)phenyl]-1H-pyrazole-1- |
| | carboxamide |
| 2078 | 5-cyclopentyl-3-[2-hydroxy-5-(propionylamino)phenyl]-N-propyl-1H-pyrazole-1- |
| | carboxamide |
| 2079 | N-benzyl-5-cyclopentyl-3-[2-hydroxy-5-(propionylamino)phenyl]-1H-pyrazole-1- |
| | carboxamide |
| 2080 | 5-cyclopentyl-3-[2-hydroxy-5-(propionylamino)phenyl]-N-(2-phenylethyl)-1H- |
| | pyrazole-1-carboxamide |
| 2081 | 5-cyclopentyl-3-[2-hydroxy-5-(propionylamino)phenyl]-N-(2-methylbenzyl)-1H- |
| | pyrazole-1-carboxamide |
| 2082 | 5-cyclopentyl-3-[2-hydroxy-5-propionylamino)phenyl]-N-(3-methylbenyl)-1H- |
| | pyrazole-1-carboxamide |
| 2083 | 5-cyclopentyl-3-[2-hydroxy-5-(propionylamino)phenyl]-N-(4-methylbenzyl)-1H- |
| | pyrazole-1-carboxamide |
| 2084 | N-(2-chlorobenzyl)-5-cyclopentyl-3-[2-hydroxy-5-(propionylamino)phenyl]-1H- |
| | pyrazole-1-carboxamide |
| 2085 | 5-cyclopentyl-N-(4-fluorobenzyl)-3-[2-hydroxy-5-(propionylamino)phenyl]-1H- |
| | pyrazole-1-carboxamide |
| 2086 | 5-cyclopentyl-N-(3,4-dichlorobenzyl)-3-[2-hydroxy-5-(propionylamino)phenyl]- |
| | 1H-pyrazole-1-carboxamide |
| 2087 | 5-cyclopentyl-3-[2-hydroxy-5-(propionylamino)phenyl]-N-(4-methoxybenzyl)- |
| | 1H-pyrazole-1-carboxamide |
| 2088 | N-(sec-butyl)-5-cyclopentyl-3-[2-hydroxy-5-(propionylamino)phenyl]-1H- |
| | pyrazole-1-carboxamide |
| 2089 | 5-cyclopentyl-N-(2-fluorobenzyl)-3-[2-hydroxy-5-(propionylamino)phenyl]-1H- |
| | pyrazole-1-carboxamide |
| 2090 | 5-cyclopentyl-N-(3-fluorobenzyl)-3-[2-hydroxy-5-(propionylamino)phenyl]-1H- |
| | pyrazole-1-carboxamide |
| 2091 | 5-cyclopentyl-N-cyclopropyl-3-[2-hydroxy-5-(propionylamino)phenyl]-1H- |
| | pyrazole-1-carboxamide |
| 2092 | 5-cyclopropyl-3-{5-[(cyclopropylcarbonyl)amino]-2-hydroxyphenyl}-N- |
| | isopropyl-1H-pyrazole-1-carboxamide |
| 2093 | 5-cyclopropyl-3-{5-[(cyclopropylcarbonyl)amino]-2-hydroxyphenyl}-N-ethyl-1H- |
| | pyrazole-1-carboxamide |
| 2094 | N-allyl-5-cyclopropyl-3-{5-[(cyclopropylcarbonyl)amino]-2-hydroxyphenyl}-1H- |
| | pyrazole-1-carboxamide |
| 2095 | 5-cyclopropyl-3-{5-[(cyclopropylcarbonyl)amino]-2-hydroxyphenyl}-N-propyl- |
| | 1H-pyrazole-1-carboxamide |
| 2096 | N-benzyl-5-cyclopropyl-3-{5-[(cyclopropylcarbonyl)amino]-2-hydroxyphenyl}- |
| | 1H-pyrazole-1-carboxamide |
| 2097 | 5-cyclopropyl-3-{5-[(cyclopropylcarbonyl)amino]-2-hydroxyphenyl}-N-(2- |
| | phenylethyl)-1H-pyrazole-1-carboxamide |
| 2098 | 5-cyclopropyl-3-{5-[(cyclopropylcarbonyl)amino]-2-hydroxyphenyl}-N-(2- |
| | methylbenzyl)-1H-pyrazole-1-carboxamide |
| 2099 | 5-cyclopropyl-3-{5-[(cyclopropylcarbonyl)amino]-2-hydroxyphenyl}-N-(3- |
| | methylbenzyl)-1H-pyrazole-1-carboxamide |
| 2100 | 5-cyclopropyl-3-{5-[(cyclopropylcarbonyl)amino]-2-hydroxyphenyl}-N-(4- |
| | methylbenzyl)-1H-pyrazole-1-carboxamide |
| 2101 | N-(2-chlorobenzyl)-5-cyclopropyl-3-{5-[(cyclopropylcarbonyl)amino]-2- |
| | Hydroxyphenyl}-1H-pyrazole-1-carboxamide |
| 2102 | 5-cyclopropyl-3-{5-[(cyclopropylcarbonyl)amino]-2-hydroxyphenyl}-N-(4- |
| | fluorobenzyl)-1H-pyrazole-1-carboxamide |
| 2103 | 5-cyclopropyl-3-{5-[(cyclopropylcarbonyl)amino]-2-hydroxyphenyl}-N-(3,4- |
| | dichlorobenzyl)-1H-pyrazole-1-carboxamide |
| 2104 | 5-cyclopropyl-3-{5-[(cyclopropylcarbonyl)amino]-2-hydroxyphenyl}-N-(4- |
| | methoxybenzyl)-1H-pyrazole-1-carboxamide |
| 2105 | N-(sec-butyl)-5-cyclopropyl-3-{5-[(cyclopropylcarbonyl)amino]-2- |
| | hydroxyphenyl}-1H-pyrazole-1-carboxamide |
| 2106 | 5-cyclopropyl-3-{5-[(cyclopropylcarbonyl)amino]-2-hydroxyphenyl}-N-(2- |
| | fluorobenzyl)-1H-pyrazole-1-carboxamide |
| 2107 | 5-cyclopropyl-3-{5-[(cyclopropylcarbonyl)amino]-2-hydroxyphenyl}-N-(3- |
| | fluorobenzyl)-1H-pyrazole-1-carboxamide |
| 2108 | N,5-dicyclopropyl-3-{5-[(cyclopropylcarbonyl)amino]-2-hydroxyphenyl}-1H- |
| | pyrazole-1-carboxamide |
| 2109 | 5-cyclobutyl-3-{5-[(cyclopropylcarbonyl)amino]-2-hydroxyphenyl}-N-isopropyl- |
| | 1H-pyrazole-1-carboxamide |
| 2110 | 5-cyclobutyl-3-{5-[(cyclopropylcarbonyl)amino]-2-hydroxyphenyl}-N-ethyl-1H- |
| | pyrazole-1-carboxamide |
| 2111 | N-allyl-5-cyclobutyl-3-{5-[(cyclopropylcarbonyl)amino]-2-hydroxyphenyl}-1H- |
| | pyrazole-1-carboxamide |
| 2112 | 5-cyclobutyl-3-{5-[(cyclopropylcarbonyl)amino]-2-hydroxyphenyl}-N-propyl-1H- |
| | pyrazole-1-carboxamide |
| 2113 | N-benzyl-5-cyclobutyl-3-{5-[(cyclopropylcarbonyl)amino]-2-hydroxyphenyl}-1H- |
| | pyrazole-1-carboxamide |
| 2114 | 5-cyclobutyl-3-{5-[(cyclopropylcarbonyl)amino]-2-hydroxyphenyl}-N-(2- |
| | phenylethyl)-1H-pyrazole-1-carboxamide |
| 2115 | 5-cyclobutyl-3-{5-[(cyclopropylcarbonyl)amino]-2-hydroxyphenyl}-N-(2- |
| | methylbenzyl)-1H-pyrazole-1-carboxamide |
| 2116 | 5-cyclobutyl-3-{5-[(cyclopropylcarbonyl)amino]-2-hydroxyphenyl}-N-(3- |
| | methylbenzyl)-1H-pyrazole-1-carboxamide |
| 2117 | 5-cyclobutyl-3-{5-[(cyclopropylcarbonyl)amino]-2-hydroxyphenyl}-N-(4- |
| | methylbenzyl)-1H-pyrazole-1-carboxamide |
| 2118 | N-(2-chlorobenzyl)-5-cyclobutyl-3-{5-[(cyclopropylcarbonyl)amino]-2- |
| | hydroxyphenyl}-1H-pyrazole-1-carboxamide |
| 2119 | 5-cyclobutyl-3-{5-[(cyclopropylcarbonyl)amino]-2-hydroxyphenyl}-N-(4- |
| | fluorobenzyl)-1H-pyrazole-1-carboxamide |
| 2120 | 5-cyclobutyl-3-{5-[(cyclopropylcarbonyl)amino]-2-hydroxyphenyl}-N-(3,4- |
| | dichlorobenzyl)-1H-pyrazole-1-carboxamide |
| 2121 | 5-cyclobutyl-3-{5-[(cyclopropylcarbonyl)amino]-2-hydroxyphenyl}-N-(4- |
| | methoxybenzyl)-1H-pyrazole-1-carboxamide |
| 2122 | N-(sec-butyl)-5-cyclobutyl-3-{5-[(cyclopropylcarbonyl)amino]-2- |
| | hydroxyphenyl}-1H-pyrazole-1-carboxamide |
| 2123 | 5-cyclobutyl-3-{5-[(cyclopropylcarbonyl)amino]-2-hydroxyphenyl}-N-(2- |
| | fluorobenzyl)-1H-pyrazole-1-carboxamide |
| 2124 | 5-cyclobutyl-3-{5-[(cyclopropylcarbonyl)amino]-2-hydroxyphenyl}-N-(3- |
| | fluorobenzyl)-1H-pyrazole-1-carboxamide |
| 2125 | 5-cyclobutyl-N-cyclopropyl-3-{5-[(cyclopropylcarbonyl)amino]-2- |
| | hydroxyphenyl}-1H-pyrazole-1-carboxamide |
| 2126 | 5-cyclopentyl-3-{5-[(cyclopropylcarbonyl)amino]-2-hydroxyphenyl}-N-isopropyl- |
| | 1H-pyrazole-1-carboxamide |
| 2127 | 5-cyclopentyl-3-{5-[(cyclopropylcarbonyl)amino]-2-hydroxyphenyl}-N-ethyl-1H- |
| | pyrazole-1-carboxamide |
| 2128 | N-allyl-5-cyclopentyl-3-{5-[(cyclopropylcarbonyl)amino]-2-hydroxyphenyl}-1H- |
| | pyrazole-1-carboxamide |
| 2129 | 5-cyclopentyl-3-{5-[(cyclopropylcarbonyl)amino]-2-hydroxyphenyl}-N-propyl- |
| | 1H-pyrazole-1-carboxamide |
| 2130 | N-benzyl-5-cyclopentyl-3-{5-[(cyclopropylcarbonyl)amino]-2-hydroxyphenyl}- |
| | 1H-pyrazole-1-carboxamide |
| 2131 | 5-cyclopentyl-3-{5-[(cyclopropylcarbonyl)amino]-2-hydroxyphenyl}-N-(2- |
| | phenylethyl)-1H-pyrazole-1-carboxamide |
| 2132 | 5-cyclopentyl-3-{5-[(cyclopropylcarbonyl)amino]-2-hydroxyphenyl}-N-(2- |
| | methylbenzyl)-1H-pyrazole-1-carboxamide |
| 2133 | 5-cyclopentyl-3-{5-[(cyclopropylcarbonyl)amino]-2-hydroxyphenyl}-N-(3- |
| | methylbenzyl)-1H-pyrazole-1-carboxamide |
| 2134 | 5-cyclopentyl-3-{5-[(cyclopropylcarbonyl)amino]-2-hydroxyphenyl}-N-(4- |
| | methylbenzyl)-1H-pyrazole-1-carboxamide |
| 2135 | N-(2-chlorobenzyl)-5-cyclopentyl-3-{5-[(cyclopropylcarbonyl)amino]-2- |
| | hydroxyphenyl}-1H-pyrazole-1-carboxamide |
| 2136 | 5-cyclopentyl-3-{5-[(cyclopropylcarbonyl)amino]-2-hydroxyphenyl}-N-(4- |
| | fluorobenzyl)-1H-pyrazole-1-carboxamide |
| 2137 | 5-cyclopentyl-3-{5-[(cyclopropylcarbonyl)amino]-2-hydroxyphenyl}-N-(3,4- |
| | dichlorobenzyl)-1H-pyrazole-1-carboxamide |
| 2138 | 5-cyclopentyl-3-{5-[(cyclopropylcarbonyl)amino]-2-hydroxyphenyl}-N-(4- |
| | methoxybenzyl)-1H-pyrazole-1-carboxamide |
| 2139 | N-(sec-butyl)-5-cyclopentyl-3-{5-[(cyclopropylcarbonyl)amino]-2- |
| | hydroxyphenyl}-1H-pyrazole-1-carboxamide |
| 2140 | 5-cyclopentyl-3-{5-[(cyclopropylcarbonyl)amino]-2-hydroxyphenyl}-N-(2- |
| | fluorobenzyl)-1H-pyrazole-1-carboxamide |
| 2141 | 5-cyclopentyl-3-{5-[(cyclopropylcarbonyl)amino]-2-hydroxyphenyl}-N-(3- |
| | fluorobenzyl)-1H-pyrazole-1-carboxamide |
| 2142 | 5-cyclopentyl-N-cyclopropyl-3-{5-[(cyclopropylcarbonyl)amino]-2- |
| | hydroxyphenyl}-1H-pyrazole-1-carboxamide |
| 2143 | 5-cyclopropyl-3-[5-(2-furoylamino)-2-hydroxyphenyl]-N-isopropyl-1H-pyrazole- |
| | 1-carboxamide |
| 2144 | 5-cyclopropyl-N-ethyl-3-[5-(2-furoylamino)-2-hydroxyphenyl]-1H-pyrazole-1- |
| | carboxamide |
| 2145 | N-allyl-5-cyclopropyl-3-[5-(2-furoylamino)-2-hydroxyphenyl]-1H-pyrazole-1- |
| | carboxamide |
| 2146 | 5-cyclopropyl-3-[5-(2-furoylamino)-2-hydroxyphenyl]-N-propyl-1H-pyrazole-1- |
| | carboxamide |
| 2147 | N-benzyl-5-cyclopropyl-3-[5-(2-furoylamino)-2-hydroxyphenyl]-1H-pyrazole-1- |
| | carboxamide |
| 2148 | 5-cyclopropyl-3-[5-(2-furoylamino)-2-hydroxyphenyl]-N-(2-phenylethyl)-1H- |
| | pyrazole-1-carboxamide |
| 2149 | 5-cyclopropyl-3-[5-(2-furoylamino)-2-hydroxyphenyl]-N-(2-methylbenzyl)-1H- |
| | pyrazole-1-carboxamide |
| 2150 | 5-cyclopropyl-3-[5-(2-furoylamino)-2-hydroxyphenyl]-N-(3-methylbenzyl)-1H- |
| | pyrazole-1-carboxamide |
| 2151 | 5-cyclopropyl-3-[5-(2-furoylamino)-2-hydroxyphenyl]-N-(4-methylbenzyl)-1H- |
| | pyrazole-1-carboxamide |
| 2152 | N-(2-chlorobenzyl)-5-cyclopropyl-3-[5-(2-furoylamino)-2-hydroxyphenyl]-1H- |
| | pyrazole-1-carboxamide |
| 2153 | 5-cyclopropyl-N-(4-fluorobenzyl)-3-[5-(2-furoylamino)-2-hydroxyphenyl]-1H- |
| | pyrazole-1-carboxamide |
| 2154 | 5-cyclopropyl-N-(3,4-dichlorobenzyl)-3-[5-(2-furoylamino)-2-hydroxyphenyl]- |
| | 1H-pyrazole-1-carboxamide |
| 2155 | 5-cyclopropyl-3-[5-(2-furoylamino)-2-hydroxyphenyl]-N-(4-methoxybenzyl)-1H- |
| | pyrazole-1-carboxamide |
| 2156 | N-(sec-butyl)-5-cyclopropyl-3-[5-(2-furoylamino)-2-hydroxyphenyl]-1H-pyrazole- |
| | 1-carboxamide |
| 2157 | 5-cyclopropyl-N-(2-fluorobenzyl)-3-[5-(2-furoylamino)-2-hydroxyphenyl]-1H- |
| | pyrazole-1-carboxamide |
| 2158 | 5-cyclopropyl-N-(3-fluorobenzyl)-3-[5-(2-furoylamino)-2-hydroxyphenyl]-1H- |
| | pyrazole-1-carboxamide |
| 2159 | N,5-dicyclopropyl-3-[5-(2-furoylamino)-2-hydroxyphenyl]-1H-pyrazole-1- |
| | carboxamide |
| 2160 | 5-cyclobutyl-3-[5-(2-furoylamino)-2-hydroxyphenyl]-N-isopropyl-1H-pyrazole-1- |
| | carboxamide |
| 2161 | 5-cyclobutyl-N-ethyl-3-[5-(2-furoylamino)-2-hydroxyphenyl]-1H-pyrazole-1- |
| | carboxamide |
| 2162 | N-allyl-5-cyclobutyl-3-[5-(2-furoylamino)-2-hydroxyphenyl]-1H-pyrazole-1- |
| | carboxamide |
| 2163 | 5-cyclobutyl-3-[5-(2-furoylamino)-2-hydroxyphenyl]-N-propyl-1H-pyrazole-1- |
| | carboxamide |
| 2164 | N-benzyl-5-cyclobutyl-3-[5-(2-furoylamino)-2-hydroxyphenyl]-1H-pyrazole-1- |
| | carboxamide |
| 2165 | 5-cyclobutyl-3-[5-(2-furoylamino)-2-hydroxyphenyl]-N-(2-phenylethyl)-1H- |
| | pyrazole-1-carboxamide |
| 2166 | 5-cyclobutyl-3-[5-(2-furoylamino)-2-hydroxyphenyl]-N-(2-methylbenzyl)-1H- |
| | pyrazole-1-carboxamide |
| 2167 | 5-cyclobutyl-3-[5-(2-furoylamino)-2-hydroxyphenyl]-N-(3-methylbenzyl)-1H- |
| | pyrazole-1-carboxamide |
| 2168 | 5-cyclobutyl-3-[5-(2-furoylamino)-2-hydroxyphenyl]-N-(4-methylbenzyl)-1H- |
| | pyrazole-1-carboxamide |
| 2169 | N-(2-chlorobenzyl)-5-cyclobutyl-3-[5-(2-furoylamino)-2-hydroxyphenyl]-1H- |
| | pyrazole-1-carboxamide |
| 2170 | 5-cyclobutyl-N-(4-fluorobenzyl)-3-[5-(2-furoylamino)-2-hydroxyphenyl]-1H- |
| | pyrazole-1-carboxamide |
| 2171 | 5-cyclobutyl-N-(3,4-dichlorobenzyl)-3-[5-(2-furoylamino)-2-hydroxyphenyl]-1H- |
| | pyrazole-1-carboxamide |
| 2172 | 5-cyclobutyl-3-[5-(2-furoylamino)-2-hydroxyphenyl]-N-(4-methoxybenzyl)-1H- |
| | pyrazole-1-carboxamide |
| 2173 | N-(sec-butyl)-5-cyclobutyl-3-[5-(2-furoylamino)-2-hydroxyphenyl]-1H-pyrazole- |
| | 1-carboxamide |
| 2174 | 5-cyclobutyl-N-(2-fluorobenzyl)-3-[5-(2-furoylamino)-2-hydroxyphenyl]-1H- |
| | pyrazole-1-carboxamide |
| 2175 | 5-cyclobutyl-N-(3-fluorobenzyl)-3-[5-(2-furoylamino)-2-hydroxyphenyl]-1H- |
| | pyrazole-1-carboxamide |
| 2176 | 5-cyclobutyl-N-cyclopropyl-3-[5-(2-furoylamino)-2-hydroxyphenyl]-1H-pyrazole- |
| | 1-carboxamide |
| 2177 | 5-cyclopentyl-3-[5-(2-furoylamino)-2-hydroxyphenyl]-N-isopropyl-1H-pyrazole- |
| | 1-carboxamide |
| 2178 | 5-cyclopentyl-N-ethyl-3-[5-(2-furoylamino)-2-hydroxyphenyl]-1H-pyrazole-1- |
| | carboxamide |
| 2179 | N-allyl-5-cyclopentyl-3-[5-(2-furoylamino)-2-hydroxyphenyl]-1H-pyrazole-1- |
| | carboxamide |
| 2180 | 5-cyclopentyl-3-[5-(2-furoylamino)-2-hydroxyphenyl]-N-propyl-1H-pyrazole-1- |
| | carboxamide |
| 2181 | N-benzyl-5-cyclopentyl-3-[5-(2-furoylamino)-2-hydroxyphenyl]-1H-pyrazole-1- |
| | carboxamide |
| 2182 | 5-cyclopentyl-3-[5-(2-furoylamino)-2-hydroxyphenyl]-N-(2-phenylethyl)-1H- |
| | pyrazole-1-carboxamide |
| 2183 | 5-cyclopentyl-3-[5-(2-furoylamino)-2-hydroxyphenyl]-N-(2-methylbenzyl)-1H- |
| | pyrazole-1-carboxamide |
| 2184 | 5-cyclopentyl-3-[5-(2-furoylamino)-2-hydroxyphenyl]-N-(3-methylbenzyl)-1H- |
| | pyrazole-1-carboxamide |
| 2185 | 5-cyclopentyl-3-[5-(2-furoylamino)-2-hydroxyphenyl]-N-(4-methylbenzyl)-1H- |
| | pyrazole-1-carboxamide |
| 2186 | N-(2-chlorobenzyl)-5-cyclopentyl-3-[5-(2-furoylamino)-2-hydroxyphenyl]-1H- |
| | pyrazole-1-carboxamide |
| 2187 | 5-cyclopentyl-N-(4-fluorobenzyl)-3-[5-(2-furoylamino)-2-hydroxyphenyl]-1H- |
| | pyrazole-1-carboxamide |
| 2188 | 5-cyclopentyl-N-(3,4-dichlorobenzyl)-3-[5-(2-furoylamino)-2-hydroxyphenyl]- |
| | 1H-pyrazole-1-carboxamide |
| 2189 | 5-cyclopentyl-3-[5-(2-furoylamino)-2-hydroxyphenyl]-N-(4-methoxybenzyl)-1H- |
| | pyrazole-1-carboxamide |
| 2190 | N-(sec-butyl)-5-cyclopentyl-3-[5-(2-furoylamino)-2-hydroxyphenyl]-1H-pyrazole- |
| | 1-carboxamide |
| 2191 | 5-cyclopentyl-N-(2-fluorobenzyl)-3-[5-(2-furoylamino)-2-hydroxyphenyl]-1H- |
| | pyrazole-1-carboxamide |
| 2192 | 5-cyclopentyl-N-(3-fluorobenzyl)-3-[5-(2-furoylamino)-2-hydroxyphenyl]-1H- |
| | pyrazole-1-carboxamide |
| 2193 | 5-cyclopentyl-N-cyclopropyl-3-[5-(2-furoylamino)-2-hydroxyphenyl]-1H- |
| | pyrazole-1-carboxamide |
| 2194 | 5-cyclopropyl-3-{2-hydroxy-5-[(thien-2-ylcarbonyl)amino]phenyl}-N-isopropyl- |
| | 1H-pyrazole-1-carboxamide |
| 2195 | 5-cyclopropyl-N-ethyl-3-{2-hydroxy-5-[(thien-2-ylcarbonyl)amino]phenyl}-1H- |
| | pyrazole-1-carboxamide |
| 2196 | N-allyl-5-cyclopropyl-3-{2-hydroxy-5-[(thien-2-ylcarbonyl)amino]phenyl}-1H- |
| | pyrazole-1-carboxamide |
| 2197 | 5-cylopropyl-3-{2-hydroxy-5-[(thien-2-ylcarbonyl)amino]phenyl}-N-propyl-1H- |
| | pyrazole-1-carboxamide |
| 2198 | N-benyl-5-cyclopropyl-3-{2-hydroxy-5-[(thien-2-ylcarbonyl)amino]phenyl}-1H- |
| | pyrazole-1-carboxamide |
| 2199 | 5-cyclopropyl-3-{2-hydroxy-5-[(thien-2-ylcarbonyl)amino]phenyl}-N-(2- |
| | phenylethyl)-1H-pyrazole-1-carboxamide |
| 2200 | 5-cyclopropyl-3-{2-hydroxy-5-[(thien-2-ylcarbonyl)amino]phenyl}-N-(2- |
| | methylbenzyl)-1H-pyrazole-1-carboxamide |
| 2201 | 5-cyclopropyl-3-{2-hydroxy-5-[(thien-2-ylcarbonyl)amino]phenyl}-N-(3- |
| | methylbenzyl)-1H-pyrazole-1-carboxamide |
| 2202 | 5-cyclopropyl-3-{2-hydroxy-5-[(thien-2-ylcarbonyl)amino]phenyl}-N-(4- |
| | methylbenzyl)-1H-pyrazole-1-carboxamide |
| 2203 | N-(2-chlorobenzyl)-5-cyclopropyl-3-{2-hydroxy-5-[(thien-2- |
| | ylcarbonyl)amino]phenyl}-1H-pyrazole-1-carboxamide |
| 2204 | 5-cyclopropyl-N-(4-fluorobenzyl)-3-{2-hydroxy-5-[(thien-2- |
| | ylcarbonyl)amino]phenyl}-1H-pyrazole-1-carboxamide |
| 2205 | 5-cyclopropyl-N-(3,4-dichlorobenzyl)-3-{2-hydroxy-5-[(thien-2- |
| | ylcarbonyl)amino]phenyl}-1H-pyrazole-1-carboxamide |
| 2206 | 5-cyclopropyl-3-{2-hydroxy-5-[(thien-2-ylcarbonyl)amino]phenyl}-N-(4- |
| | methoxybenzyl)-1H-pyrazole-1-carboxamide |
| 2207 | N-(sec-butyl)-5-cyclopropyl-3-{2-hydroxy-5-[(thien-2-ylcarbonyl)amino]phenyl}- |
| | 1H-pyrazole-1-carboxamide |
| 2208 | 5-cyclopropyl-N-(2-fluorobenzyl)-3-{2-hydroxy-5-[(thien-2- |
| | ylcarbonyl)amino]phenyl}-1H-pyrazole-1-carboxamide |
| 2209 | 5-cyclopropyl-N-(3-fluorobenzyl)-3-{2-hydroxy-5-[(thien-2- |
| | ylcarbonyl)amino]phenyl}-1H-pyrazole-1-carboxamide |
| 2210 | N,5-dicyclopropyl-3-{2-hydroxy-5-[(thien-2-ylcarbonyl)amino]phenyl}-1H- |
| | pyrazole-1-carboxamide |
| 2211 | 5-cyclobutyl-3-{2-hydroxy-5-[(thien-2-ylcarbonyl)amino]phenyl}-N-isopropyl- |
| | 1H-pyrazole-1-carboxamide |
| 2212 | 5-cyclobutyl-N-ethyl-3-{2-hydroxy-5-[(thien-2-ylcarbonyl)amino]phenyl}-1H- |
| | pyrazole-1-carboxamide |
| 2213 | N-allyl-5-cyclobutyl-3-{2-hydroxy-5-[(thien-2-ylcarbonyl)amino]phenyl}-1H- |
| | pyrazole-1-carboxamide |
| 2214 | 5-cyclobutyl-3-{2-hydroxy-5-[(thien-2-ylcarbonyl)amino]phenyl}-N-propyl-1H- |
| | pyrazole-1-carboxamide |
| 2215 | N-benzyl-5-cyclobutyl-3-{2-hydroxy-5-[(thien-2-ylcarbonyl)amino]phenyl}-1H- |
| | pyrazole-1-carboxamide |
| 2216 | 5-cyclobutyl-3-{2-hydroxy-5-[(thien-2-ylcarbonyl)amino]phenyl}-N-(2- |
| | phenylethyl)-1H-pyrazole-1-carboxamide |
| 2217 | 5-cyclobutyl-3-{2-hydroxy-5-[(thien-2-ylcarbonyl)amino]phenyl}-N-(2- |
| | methylbenzyl)-1H-pyrazole-1-carboxamide |
| 2218 | 5-cyclobutyl-3-{2-hydroxy-5-[(thien-2-ylcarbonyl)amino]phenyl}-N-(3- |
| | methylbenzyl)-1H-pyrazole-1-carboxamide |
| 2219 | 5-cyclobutyl-3-{2-hydroxy-5-[(thien-2-ylcarbonyl)amino]phenyl}-N-(4- |
| | methylbenzyl)-1H-pyrazole-1-carboxamide |
| 2220 | N-(2-chlorobenzyl)-5-cyclobutyl-3-{2-hydroxy-5-[(thien-2- |
| | ylcarbonyl)amino]phenyl}-1H-pyrazole-1-carboxamide |
| 2221 | 5-cyclobutyl-N-(4-fluorobenzyl)-3-{2-hydroxy-5-[(thien-2- |
| | ylcarbonyl)amino]phenyl}-1H-pyrazole-1-carboxamide |
| 2222 | 5-cyclobutyl-N-(3,4-dichlorobenzyl)-3-{2-hydroxy-5-[(thien-2- |
| | ylcarbonyl)amino]phenyl}-1H-pyrazole-1-carboxamide |
| 2223 | 5-cyclobutyl-3-{2-hydroxy-5-[(thien-2-ylcarbonyl)amino]phenyl}-N-(4- |
| | methoxybenzyl)-1H-pyrazole-1-carboxamide |
| 2224 | N-(sec-butyl)-5-cyclobutyl-3-{2-hydroxy-5-[(thien-2-ylcarbonyl)amino]phenyl}- |
| | 1H-pyrazole-1-carboxamide |
| 2225 | 5-cyclobutyl-N-(2-fluorobenzyl)-3-{2-hydroxy-5-[(thien-2- |
| | ylcarbonyl)amino]phenyl}-1H-pyrazole-1-carboxamide |
| 2226 | 5-cyclobutyl-N-(3-fluorobenzyl)-3-{2-hydroxy-5-[(thien-2- |
| | ylcarbonyl)amino]phenyl}-1H-pyrazole-1-carboxamide |
| 2227 | 5-cyclobutyl-N-cyclopropyl-3-{2-hydroxy-5-[(thien-2-ylcarbonyl)amino]phenyl}- |
| | 1H-pyrazole-1-carboxamide |
| 2228 | 5-cyclopentyl-3-{2-hydroxy-5-[(thien-2-ylcarbonyl)amino]phenyl}-N-isopropyl- |
| | 1H-pyrazole-1-carboxamide |
| 2229 | 5-cyclopentyl-N-ethyl-3-{2-hydroxy-5-[(thien-2-ylcarbonyl)amino]phenyl}-1H- |
| | pyrazole-1-carboxamide |
| 2230 | N-allyl-5-cyclopentyl-3-{2-hydroxy-5-[(thien-2-ylcarbonyl)amino]phenyl}-1H- |
| | pyrazole-1-carboxamide |
| 2231 | 5-cyclopentyl-3-{2-hydroxy-5-[(thien-2-ylcarbonyl)amino]phenyl}-N-propyl-1H- |
| | pyrazole-1-carboxamide |
| 2232 | N-benzyl-5-cyclopentyl-3-{2-hydroxy-5-[(thien-2-ylcarbonyl)amino]phenyl}-1H- |
| | pyrazole-1-carboxamide |
| 2233 | 5-cyclopentyl-3-{2-hydroxy-5-[(thien-2-ylcarbonyl)amino]phenyl}-N-(2- |
| | phenylethyl)-1H-pyrazole-1-carboxamide |
| 2234 | 5-cyclopentyl-3-{2-hydroxy-5-[(thien-2-ylcarbonyl)amino]phenyl}-N-(2- |
| | methylbenzyl)-1H-pyrazole-1-carboxamide |
| 2235 | 5-cyclopentyl-3-{2-hydroxy-5-[(thien-2-ylcarbonyl)amino]phenyl}-N-(3- |
| | methylbenzyl)-1H-pyrazole-1-carboxamide |
| 2236 | 5-cyclopentyl-3-{2-hydroxy-5-[(thien-2-ylcarbonyl)amino]phenyl}-N-(4- |
| | methylbenzyl)-1H-pyrazole-1-carboxamide |
| 2237 | N-(2-chlorobenzyl)-5-cyclopentyl-3-{2-hydroxy-5-[(thien-2- |
| | ylcarbonyl)amino]phenyl}-1H-pyrazole-1-carboxamide |
| 2238 | 5-cyclopentyl-N-(4-fluorobenzyl)-3-{2-hydroxy-5-[(thien-2- |
| | ylcarbonyl)amino]phenyl}-1H-pyrazole-1-carboxamide |
| 2239 | 5-cyclopentyl-N-(3,4-dichlorobenzyl)-3-{2-hydroxy-5-[(thien-2- |
| | ylcarbonyl)amino]phenyl}-1H-pyrazole-1-carboxamide |
| 2240 | 5-cyclopentyl-3-{2-hydroxy-5-[(thien-2-ylcarbonyl)amino]phenyl}-N-(4- |
| | methoxybenzyl)-1H-pyrazole-1-carboxamide |
| 2241 | N-(sec-butyl)-5-cyclopentyl-3-{2-hydroxy-5-[(thien-2-ylcarbonyl)amino]phenyl}- |
| | 1H-pyrazole-1-carboxamide |
| 2242 | 5-cyclopentyl-N-(2-fluorobenzyl)-3-{2-hydroxy-5-[(thien-2- |
| | ylcarbonyl)amino]phenyl}-1H-pyrazole-1-carboxamide |
| 2243 | 5-cyclopentyl-N-(3-fluorobenzyl)-3-{2-hydroxy-5-[(thien-2- |
| | ylcarbonyl)amino]phenyl}-1H-pyrazole-1-carboxamide |
| 2244 | 5-cyclopentyl-N-cyclopropyl-3-{2-hydroxy-5-[(thien-2- |
| | ylcarbonyl)amino]phenyl}-1H-pyrazole-1-carboxamide |
| 2245 | N-(3-{5-cyclopropyl-1-[(isopropylamino)carbonyl]-1H-pyrazol-3-yl}-4- |
| | hydroxyphenyl)quinoxaline-2-carboxamide |
| 2246 | N-(3-{5-cyclopropyl-1-[(ethylamino)carbonyl]-1H-pyrazol-3-yl}-4- |
| | hydroxyphenyl)quinoxaline-2-carboxamide |
| 2247 | N-(3-{1-[(allylamino)carbonyl]-5-cyclopropyl-1H-pyrazol-3-yl}-4- |
| | hydroxyphenyl)quinoxaline-2-carboxamide |
| 2248 | N-(3-{5-cyclopropyl-1-[(propylamino)carbonyl]-1H-pyrazol-3-yl}-4- |
| | hydroxyphenyl)quinoxaline-2-carboxamide |
| 2249 | N-(3-{1-[(benzylamino)carbonyl]-5-cyclopropyl-1H-pyrazol-3-yl}-4- |
| | hydroxyphenyl)quinoxaline-2-carboxamide |
| 2250 | N-[3-(5-cyclopropyl-1-{[(2-phenylethyl)amino]carbonyl}-1H-pyrazol-3-yl)-4- |
| | hydroxyphenyl]quinoxaline-2-carboxamide |
| 2251 | N-[3-(5-cyclopropyl-1-{[(2-methylbenzyl)amino]carbonyl}-1H-pyrazol-3-yl)-4- |
| | hydroxyphenyl]quinoxaline-2-carboxamide |
| 2252 | N-[3-(5-cyclopropyl-1-{[(3-methylbenzyl)amino]carbonyl}-1H-pyrazol-3-yl)-4- |
| | hydroxyphenyl]quinoxaline-2-carboxamide |
| 2253 | N-[3-(5-cyclopropyl-1-{[(4-methylbenzyl)amino]carbonyl}-1H-pyrazol-3-yl)-4- |
| | hydroxyphenyl]quinoxaline-2-carboxamide |
| 2254 | N-[3-(1-{[(2-chlorobenzyl)amino]carbonyl}-5-cyclopropyl-1H-pyrazol-3-yl)-4- |
| | hydroxyphenyl]quinoxaline-2-carboxamide |
| 2255 | N-[3-(5-cyclopropyl-1-{[(4-fluorobenzyl)amino]carbonyl}-1H-pyrazol-3-yl)-4- |
| | hydroxyphenyl]quinoxaline-2-carboxamide |
| 2256 | N-[3-(5-cyclopropyl-1-{[(3,4-dichlorobenzyl)amino]carbonyl}-1H-pyrazol-3-yl)- |
| | 4-hydroxyphenyl]quinoxaline-2-carboxamide |
| 2257 | N-[3-(5-cyclopropyl-1-{[(4-methoxybenzyl)amino]carbonyl}-1H-pyrazol-3-yl)-4- |
| | hydroxyphenyl]quinoxaline-2-carboxamide |
| 2258 | N-(3-{1-[(sec-butylamino)carbonyl]-5-cyclopropyl-1H-pyrazol-3-yl}-4- |
| | hydroxyphenyl)quinoxaline-2-carboxamide |
| 2259 | N-[3-(5-cyclopropyl-1-{[(2-fluorobenzyl)amino]carbonyl}-1H-pyrazol-3-yl)-4- |
| | hydroxyphenyl]quinoxaline-2-carboxamide |
| 2260 | N-[3-(5-cyclopropyl-1-{[(3-fluorobenzyl)amino]carbonyl}-1H-pyrazol-3-yl)-4- |
| | hydroxyphenyl]quinoxaline-2-carboxamide |
| 2261 | N-(3-{5-cyclopropyl-1-[(cyclopropylamino)carbonyl]-1H-pyrazol-3-yl}-4- |
| | hydroxyphenyl)quinoxaline-2-carboxamide |
| 2262 | N-(3-{5-cyclobutyl-1-[(isopropylamino)carbonyl]-1H-pyrazol-3-yl}-4- |
| | hydroxyphenyl)quinoxaline-2-carboxamide |
| 2263 | N-(3-{5-cyclobutyl-1-[(ethylamino)carbonyl]-1H-pyrazol-3-yl}-4- |
| | hydroxyphenyl)quinoxaline-2-carboxamide |
| 2264 | N-(3-{1-[(allylamino)carbonyl]-5-cyclobutyl-1H-pyrazol-3-yl}-4- |
| | hydroxyphenyl)quinoxaline-2-carboxamide |
| 2265 | N-(3-{5-cyclobutyl-1-[(propylamino)carbonyl]-1H-pyrazol-3-yl}-4- |
| | hydroxyphenyl)quinoxaline-2-carboxamide |
| 2266 | N-(3-{1-[(benzylamino)carbonyl]-5-cyclobutyl-1H-pyrazol-3-yl}-4- |
| | hydroxyphenyl)quinoxaline-2-carboxamide |
| 2267 | N-[3-(5-cyclobutyl-1-{[(2-phenylethyl)amino]carbonyl}-1H-pyrazol-3-yl)-4- |
| | hydroxyphenyl]quinoxaline-2-carboxamide |
| 2268 | N-[3-(5-cyclobutyl-1-{[(2-methylbenzyl)amino]carbonyl}-1H-pyrazol-3-yl)-4- |
| | hydroxyphenyl]quinoxaline-2-carboxamide |
| 2269 | N-[3-(5-cyclobutyl-1-{[(3-methylbenzyl)amino]carbonyl}-1H-pyrazol-3-yl)-4- |
| | hydroxyphenyl]quinoxaline-2-carboxamide |
| 2270 | N-[3-(5-cyclobutyl-1-{[(4-methylbenzyl)amino]carbonyl}-1H-pyrazol-3-yl)-4- |
| | hydroxyphenyl]quinoxaline-2-carboxamide |
| 2271 | N-[3-(1-{[(2-chlorobenzyl)amino]carbonyl}-5-cyclobutyl-1H-pyrazol-3-yl)-4- |
| | hydroxyphenyl]quinoxaline-2-carboxamide |
| 2272 | N-[3-(5-cyclobutyl-1-{[(4-fluorobenzyl)amino]carbonyl}-1H-pyrazol-3-yl)-4- |
| | hydroxyphenyl]quinoxaline-2-carboxamide |
| 2273 | N-[3-(5-cyclobutyl-1-{[(3,4-dichlorobenzyl)amino]carbonyl}-1H-pyrazol-3-yl)-4- |
| | hydroxyphenyl]quinoxaline-2-carboxamide |
| 2274 | N-[3-(5-cyclobutyl-1-{[(4-methoxybenzyl)amino]carbonyl}-1H-pyrazol-3-yl)-4- |
| | hydroxyphenyl]quinoxaline-2-carboxamide |
| 2275 | N-(3-{1-[(sec-butylamino)carbonyl]-5-cyclobutyl-1H-pyrazol-3-yl}-4- |
| | hydroxyphenyl)quinoxaline-2-carboxamide |
| 2276 | N-[3-(5-cyclobutyl-1-{[(2-fluorobenzyl)amino]carbonyl}-1H-pyrazol-3-yl)-4- |
| | hydroxyphenyl]quinoxaline-2-carboxamide |
| 2277 | N-[3-(5-cyclobutyl-1-{[(3-fluorobenzyl)amino]carbonyl}-1H-pyrazol-3-yl)-4- |
| | hydroxyphenyl]quinoxaline-2-carboxamide |
| 2278 | N-(3-{5-cyclobutyl-1-[(cyclopropylamino)carbonyl]-1H-pyrazol-3-yl}-4- |
| | hydroxyphenyl)quinoxaline-2-carboxamide |
| 2279 | N-(3-{5-cyclopentyl-1-[(isopropylamino)carbonyl]-1H-pyrazol-3-yl}-4- |
| | hydroxyphenyl)quinoxaline-2-carboxamide |
| 2280 | N-(3-{5-cyclopentyl-1-[(ethylamino)carbonyl]-1H-pyrazol-3-yl}-4- |
| | hydroxyphenyl)quinoxaline-2-carboxamide |
| 2281 | N-(3-{1-[(allylamino)carbonyl]-5-cyclopentyl-1H-pyrazol-3-yl}-4- |
| | hydroxyphenyl)quinoxaline-2-carboxamide |
| 2282 | N-(3-{5-cyclopentyl-1-[(propylamino)carbonyl]-1H-pyrazol-3-yl}-4- |
| | hydroxyphenyl)quinoxaline-2-carboxamide |
| 2283 | N-(3-{1-[(benzylamino)carbonyl]-5-cyclopentyl-1H-pyrazol-3-yl}-4- |
| | hydroxyphenyl) quinoxaline-2-carboxamide |
| 2284 | N-[3-(5-cyclopentyl-1-{[(2-phenylethyl)amino]carbonyl}-1H-pyrazol-3-yl)-4- |
| | hydroxyphenyl]quinoxaline-2-carboxamide |
| 2285 | N-[3-(5-cyclopentyl-1-{[(2-methylbenzyl)amino]carbonyl}-1H-pyrazol-3-yl)-4- |
| | hydroxyphenyl]quinoxaline-2-carboxamide |
| 2286 | N-[3-(5-cyclopentyl-1-{[(3-methylbenzyl)amino]carbonyl}-1H-pyrazol-3-yl)-4- |
| | hydroxyphenyl]quinoxaline-2-carboxamide |
| 2287 | N-[3-(5-cyclopentyl-1-{[(4-methylbenzyl)amino]carbonyl}-1H-pyrazol-3-yl)-4- |
| | hydroxyphenyl]quinoxaline-2-carboxamide |
| 2288 | N-[3-(1-{[(2-chlorobenzyl)amino]carbonyl}-5-cyclopentyl-1H-pyrazol-3-yl)-4- |
| | hydroxyphenyl]quinoxaline-2-carboxamide |
| 2289 | N-[3-(5-cyclopentyl-1-{[(4-fluorobenzyl)amino]carbonyl}-1H-pyrazol-3-yl)-4- |
| | hydroxyphenyl]quinoxaline-2-carboxamide |
| 2290 | N-[3-(5-cyclopentyl-1-{[(3,4-dichlorobenzyl)amino]carbonyl}-1H-pyrazol-3-yl)- |
| | 4-hydroxyphenyl]quinoxaline-2-carboxamide |
| 2291 | N-[3-(5-cyclopentyl-1-{[(4-methoxybenzyl)amino]carbonyl}-1H-pyrazol-3-yl)-4- |
| | hydroxyphenyl]quinoxaline-2-carboxamide |
| 2292 | N-(3-{1-[(sec-butylamino)carbonyl]-5-cyclopentyl-1H-pyrazol-3-yl}-4- |
| | hydroxyphenyl)quinoxaline-2-carboxamide |
| 2293 | N-[3-(5-cyclopentyl-1-{[(2-fluorobenzyl)amino]carbonyl}-1H-pyrazol-3-yl)-4- |
| | hydroxyphenyl]quinoxaline-2-carboxamide |
| 2294 | N-[3-(5-cyclopentyl-1-{[(3-fluorobenzyl)amino]carbonyl}-1H-pyrazol-3-yl)-4- |
| | hydroxyphenyl]quinoxaline-2-carboxamide |
| 2295 | N-(3-{5-cyclopentyl-1-[(cyclopropylamino)carbonyl]-1H-pyrazol-3-yl}-4- |
| | hydroxyphenyl)quinoxaline-2-carboxamide |
| 2296 | N-(3-{5-cyclopropyl-1-[(isopropylamino)carbonyl]-1H-pyrazol-3-yl}-4- |
| | hydroxyphenyl)nicotinamide |
| 2297 | N-(3-{5-cyclopropyl-1-[(ethylamino)carbonyl]-1H-pyrazol-3-yl}-4- |
| | hydroxyphenyl)nicotinamide |
| 2298 | N-(3-{1-[(allylamino)carbonyl]-5-cyclopropyl-1H-pyrazol-3-yl}-4- |
| | hydroxyphenyl)nicotinamide |
| 2299 | N-(3-{5-cyclopropyl-1-[(propylamino)carbonyl]-1H-pyrazol-3-yl}-4- |
| | hydroxyphenyl)nicotinamide |
| 2300 | N-(3-{1-[(benzylamino)carbonyl]-5-cyclopropyl-1H-pyrazol-3-yl}-4- |
| | hydroxyphenyl)nicotinamide |
| 2301 | N-[3-(5-cyclopropyl-1-{[(2-phenylethyl)amino]carbonyl}-1H-pyrazol-3-yl)-4- |
| | hydroxyphenyl]nicotinamide |
| 2302 | N-[3-(5-cyclopropyl-1-{[(2-methylbenzyl)amino]carbonyl}-1H-pyrazol-3-yl)-4- |
| | hydroxyphenyl]nicotinamide |
| 2303 | N-[3-(5-cyclopropyl-1-{[(3-methylbenzyl)amino]carbonyl}-1H-pyrazol-3-yl)-4- |
| | hydroxyphenyl]nicotinamide |
| 2304 | N-[3-(5-cyclopropyl-1-{[(4-methylbenzyl)amino]carbonyl}-1H-pyrazol-3-yl)-4- |
| | hydroxyphenyl]nicotinamide |
| 2305 | N-[3-(1-{[(2-chlorobenzyl)amino]carbonyl}-5-cyclopropyl-1H-pyrazol-3-yl)-4- |
| | hydroxyphenyl]nicotinamide |
| 2306 | N-[3-(5-cyclopropyl-1-{[(4-fluorobenzyl)amino]carbonyl}-1H-pyrazol-3-yl)-4- |
| | hydroxyphenyl]nicotinamide |
| 2307 | N-[3-(5-cyclopropyl-1-{[(3,4-dichlorobenzyl)amino]carbonyl}-1H-pyrazol-3-yl)- |
| | 4-hydroxyphenyl]nicotinamide |
| 2308 | N-[3-(5-cyclopropyl-1-{[(4-methoxybenzyl)amino]carbonyl}-1H-pyrazol-3-yl)-4- |
| | hydroxyphenyl]nicotinamide |
| 2309 | N-(3-{1-[(sec-butylamino)carbonyl]-5-cyclopropyl-1H-pyrazol-3-yl}-4- |
| | hydroxyphenyl) nicotinamide |
| 2310 | N-[3-(5-cyclopropyl-1-{[(2-fluorobenzyl)amino]carbonyl}-1H-pyrazol-3-yl)-4- |
| | hydroxyphenyl]nicotinamide |
| 2311 | N-[3-(5-cyclopropyl-1-{[(3-fluorobenzyl)amino]carbonyl}-1H-pyrazol-3-yl)-4- |
| | hydroxyphenyl]nicotinamide |
| 2312 | N-(3-{5-cyclopropyl-1-[(cyclopropylamino)carbonyl]-1H-pyrazol-3-yl}-4- |
| | hydroxyphenyl)nicotinamide |
| 2313 | N-(3-{5-cyclobutyl-1-[(isopropylamino)carbonyl]-1H-pyrazol-3-yl}-4- |
| | hydroxyphenyl)nicotinamide |
| 2314 | N-(3-{5-cyclobutyl-1-[(ethylamino)carbonyl]-1H-pyrazol-3-yl}-4- |
| | hydroxyphenyl)nicotinamide |
| 2315 | N-(3-{1-[(allylamino)carbonyl]-5-cyclobutyl-1H-pyrazol-3-yl}-4- |
| | hydroxyphenyl)nicotinamide |
| 2316 | N-(3-{5-cyclobutyl-1-[(propylamino)carbonyl]-1H-pyrazol-3-yl}-4- |
| | hydroxyphenyl)nicotinamide |
| 2317 | N-(3-{1-[(benzylamino)carbonyl]-5-cyclobutyl-1H-pyrazol-3-yl}-4- |
| | hydroxyphenyl)nicotinamide |
| 2318 | N-[3-(5-cyclobutyl-1-{[(2-phenylethyl)amino]carbonyl}-1H-pyrazol-3-yl)-4- |
| | hydroxyphenyl]nicotinamide |
| 2319 | N-[3-(5-cyclobutyl-1-{[(2-methylbenzyl)amino]carbonyl}-1H-pyrazol-3-yl)-4- |
| | hydroxyphenyl]nicotinamide |
| 2320 | N-[3-(5-cyclobutyl-1-{[(3-methylbenzyl)amino]carbonyl}-1H-pyrazol-3-yl)-4- |
| | hydroxyphenyl]nicotinamide |
| 2321 | N-[3-(5-cyclobutyl-1-{[(4-methylbenzyl)amino]carbonyl}-1H-pyrazol-3-yl)-4- |
| | hydroxyphenyl]nicotinamide |
| 2322 | N-[3-( 1-{[(2-chlorobenzyl)amino]carbonyl}-5-cyclobutyl-1H-pyrazol-3-yl)-4- |
| | hydroxyphenyl]nicotinamide |
| 2323 | N-[3-(5-cyclobutyl-1-{[(4-fluorobenzyl)amino]carbonyl}-1H-pyrazol-3-yl)-4- |
| | hydroxyphenyl]nicotinamide |
| 2324 | N-[3-(5-cyclobutyl-1-{[(3,4-dichlorobenzyl)amino]carbonyl}-1H-pyrazol-3-yl)-4- |
| | hydroxyphenyl]nicotinamide |
| 2325 | N-[3-(5-cyclobutyl-1-{[(4-methoxybenzyl)amino]carbonyl}-1H-pyrazol-3-yl)-4- |
| | hydroxyphenyl]nicotinamide |
| 2326 | N-(3-{1-[(sec-butylamino)carbonyl]-5-cyclobutyl-1H-pyrazol-3-yl}-4- |
| | hydroxyphenyl)nicotinamide |
| 2327 | N-[3-(5-cyclobutyl-1-{[(2-fluorobenzyl)amino]carbonyl}-1H-pyrazol-3-yl)-4- |
| | hydroxyphenyl]nicotinamide |
| 2328 | N-[3-(5-cyclobutyl-1-{[(3-fluorobenzyl)amino]carbonyl}-1H-pyrazol-3-yl)-4- |
| | hydroxyphenyl]nicotinamide |
| 2329 | N-(3-{5-cyclobutyl-1-[(cyclopropylamino)carbonyl]-1H-pyrazol-3-yl}-4- |
| | hydroxyphenyl)nicotinamide |
| 2330 | N-(3-{5-cyclopentyl-1-[(isopropylamino)carbonyl]-1H-pyrazol-3-yl}-4- |
| | hydroxyphenyl)nicotinamide |
| 2331 | N-(3-{5-cyclopentyl-1-[(ethylamino)carbonyl]-1H-pyrazol-3-yl}-4- |
| | hydroxyphenyl)nicotinamide |
| 2332 | N-(3-{1-[(allylamino)carbonyl]-5-cyclopentyl-1H-pyrazol-3-yl}-4- |
| | hydroxyphenyl)nicotinamide |
| 2333 | N-(3-{5-cyclopentyl-1-[(propylamino)carbonyl]-1H-pyrazol-3-yl}-4- |
| | hydroxyphenyl)nicotinamide |
| 2334 | N-(3-{1-[(benzylamino)carbonyl]-5-cyclopentyl-1H-pyrazol-3-yl}-4- |
| | hydroxyphenyl)nicotinamide |
| 2335 | N-[3-(5-cyclopentyl-1-{[(2-phenylethyl)amino]carbonyl}-1H-pyrazol-3-yl)-4- |
| | hydroxyphenyl]nicotinamide |
| 2336 | N-[3-(5-cyclopentyl-1-{[(2-methylbenzyl)amino]carbonyl}-1H-pyrazol-3-yl)-4- |
| | hydroxyphenyl]nicotinamide |
| 2337 | N-[3-(5-cyclopentyl-1-{[(3-methylbenzyl)amino]carbonyl}-1H-pyrazol-3-yl)-4- |
| | hydroxyphenyl]nicotinamide |
| 2338 | N-[3-(5-cyclopentyl-1-{[(4-methylbenzyl)amino]carbonyl}-1H-pyrazol-3-yl)-4- |
| | hydroxyphenyl]nicotinamide |
| 2339 | N-[3-(1-{[(2-chlorobenzyl)amino]carbonyl}-5-cyclopentyl-1H-pyrazol-3-yl)-4- |
| | hydroxyphenyl]nicotinamide |
| 2340 | N-[3-(5-cyclopentyl-1-{[(4-fluorobenzyl)amino]carbonyl}-1H-pyrazol-3-yl)-4- |
| | hydroxyphenyl]nicotinamide |
| 2341 | N-[3-(5-cyclopentyl-1-{[(3,4-dichlorobenzyl)amino]carbonyl}-1H-pyrazol-3-yl)- |
| | 4-hydroxyphenyl]nicotinamide |
| 2342 | N-[3-(5-cyclopentyl-1-{[(4-methoxybenzyl)amino]carbonyl}-1H-pyrazol-3-yl)-4- |
| | hydroxyphenyl]nicotinamide |
| 2343 | N-(3-{1-[(sec-butylamino)carbonyl]-5-cyclopentyl-1H-pyrazol-3-yl}-4- |
| | hydroxyphenyl)nicotinamide |
| 2344 | N-[3-(5-cyclopentyl-1-{[(2-fluorobenzyl)amino]carbonyl}-1H-pyrazol-3-yl)-4- |
| | hydroxyphenyl]nicotinamide |
| 2345 | N-[3-(5-cyclopentyl-1-{[(3-fluorobenzyl)amino]carbonyl}-1H-pyrazol-3-yl)-4- |
| | hydroxyphenyl]nicotinamide |
| 2346 | N-(3-{5-cyclopentyl-1-[(cyclopropylamino)carbonyl]-1H-pyrazol-3-yl}-4- |
| | hydroxyphenyl)nicotinamide |
| 2347 | N-(3-{5-cyclopropyl-1-[(isopropylamino)carbonyl]-1H-pyrazol-3-yl}-4- |
| | hydroxyphenyl)isonicotinamide |
| 2348 | N-(3-{5-cyclopropyl-1-[(ethylamino)carbonyl]-1H-pyrazol-3-yl}-4- |
| | hydroxyphenyl)isonicotinamide |
| 2349 | N-(3-{1-[(allylamino)carbonyl]-5-cyclopropyl-1H-pyrazol-3-yl}-4- |
| | hydroxyphenyl)isonicotinamide |
| 2350 | N-(3-{5-cyclopropyl-1-[(propylamino)carbonyl]-1H-pyrazol-3-yl}-4- |
| | hydroxyphenyl)isonicotinamide |
| 2351 | N-(3-{1-[(benzylamino)carbonyl]-5-cyclopropyl-1H-pyrazol-3-yl}-4- |
| | hydroxyphenyl)isonicotinamide |
| 2352 | N-[3-(5-cyclopropyl-1-{[(2-phenylethyl)amino]carbonyl}-1H-pyrazol-3-yl)-4- |
| | hydroxyphenyl]isonicotinamide |
| 2353 | N-[3-(5-cyclopropyl-1-{[(2-methylbenzyl)amino]carbonyl}-1H-pyrazol-3-yl)-4- |
| | hydroxyphenyl]isonicotinamide |
| 2354 | N-[3-(5-cyclopropyl-1-{[(3-methylbenzyl)amino]carbonyl}-1H-pyrazol-3-yl)-4- |
| | hydroxyphenyl]isonicotinamide |
| 2355 | N-[3-(5-cyclopropyl-1-{[(4-methylbenzyl)amino]carbonyl}-1H-pyrazol-3-yl)-4- |
| | hydroxyphenyl]isonicotinamide |
| 2356 | N-[3-(1-{[(2-chlorobenzyl)amino]carbonyl}-5-cyclopropyl-1H-pyrazol-3-yl)-4- |
| | hydroxyphenyl]isonicotinamide |
| 2357 | N-[3-(5-cyclopropyl-1-{[(4-fluorobenzyl)amino]carbonyl}-1H-pyrazol-3-yl)-4- |
| | hydroxyphenyl]isonicotinamide |
| 2358 | N-[3-(5-cyclopropyl-1-{[(3,4-dichlorobenzyl)amino]carbonyl}-1H-pyrazol-3-yl)- |
| | 4-hydroxyphenyl]isonicotinamide |
| 2359 | N-[3-(5-cyclopropyl-1-{[(4-methoxybenzyl)amino]carbonyl}-1H-pyrazol-3-yl)-4- |
| | hydroxyphenyl]isonicotinamide |
| 2360 | N-(3-{1-[(sec-butylamino)carbonyl]-5-cyclopropyl-1H-pyrazol-3-yl}-4- |
| | hydroxyphenyl)isonicotinamide |
| 2361 | N-[3-(5-cyclopropyl-1-{[(2-fluorobenzyl)amino]carbonyl}-1H-pyrazol-3-yl)-4- |
| | hydroxyphenyl]isonicotinamide |
| 2362 | N-[3-(5-cyclopropyl-1-{[(3-fluorobenzyl)amino]carbonyl}-1H-pyrazol-3-yl)-4- |
| | hydroxyphenyl]isonicotinamide |
| 2363 | N-(3-{5-cyclopropyl-1-[(cyclopropylamino)carbonyl]-1H-pyrazol-3-yl}-4- |
| | hydroxyphenyl)isonicotinamide |
| 2364 | N-(3-{5-cyclobutyl-1-[(isopropylamino)carbonyl]-1H-pyrazol-3-yl}-4- |
| | hydroxyphenyl)isonicotinamide |
| 2365 | N-(3-{5-cyclobutyl-1-[(ethylamino)carbonyl]-1H-pyrazol-3-yl}-4- |
| | hydroxyphenyl)isonicotinamide |
| 2366 | N-(3-{1-[(allylamino)carbonyl]-5-cyclobutyl-1H-pyrazol-3-yl}-4- |
| | hydroxyphenyl)isonicotinamide |
| 2367 | N-(3-{5-cyclobutyl-1-[(propylamino)carbonyl]-1H-pyrazol-3-yl}-4- |
| | hydroxyphenyl)isonicotinamide |
| 2368 | N-(3-{1-[(benzylamino)carbonyl]-5-cyclobutyl-1H-pyrazol-3-yl}-4- |
| | hydroxyphenyl)isonicotinamide |
| 2369 | N-[3-(5-cyclobutyl-1-{[(2-phenylethyl)amino]carbonyl}-1H-pyrazol-3-yl)-4- |
| | hydroxyphenyl]isonicotinamide |
| 2370 | N-[3-(5-cyclobutyl-1-{[(2-methylbenzyl)amino]carbonyl}-1H-pyrazol-3-yl)-4- |
| | hydroxyphenyl]isonicotinamide |
| 2371 | N-[3-(5-cyclobutyl-1-{[(3-methylbenzyl)amino]carbonyl}-1H-pyrazol-3-yl)-4- |
| | hydroxyphenyl]isonicotinamide |
| 2372 | N-[3-(5-cyclobutyl-1-{[(4-methylbenzyl)amino]carbonyl}-1H-pyrazol-3-yl)-4- |
| | hydroxyphenyl]isonicotinamide |
| 2373 | N-[3-(1-{[(2-chlorobenzyl)amino]carbonyl}-5-cyclobutyl-1H-pyrazol-3-yl)-4- |
| | hydroxyphenyl]isonicotinamide |
| 2374 | N-[3-(5-cyclobutyl-1-{[(4-fluorobenzyl)amino]carbonyl}-1H-pyrazol-3-yl)-4- |
| | hydroxyphenyl]isonicotinamide |
| 2375 | N-[3-(5-cyclobutyl-1-{[(3,4-dichlorobenzyl)amino]carbonyl}-1H-pyrazol-3-yl)-4- |
| | hydroxyphenyl]isonicotinamide |
| 2376 | N-[3-(5-cyclobutyl-1-{[(4-methoxybenzyl)amino]carbonyl}-1H-pyrazol-3-yl)-4- |
| | hydroxyphenyl]isonicotinamide |
| 2377 | N-(3-{1-[(sec-butylamino)carbonyl]-5-cyclobutyl-1H-pyrazol-3-yl}-4- |
| | hydroxyphenyl) isonicotinamide |
| 2378 | N-[3-(5-cyclobutyl-1-{[(2-fluorobenzyl)amino]carbonyl}-1H-pyrazol-3-yl)-4- |
| | hydroxyphenyl]isonicotinamide |
| 2379 | N-[3-(5-cyclobutyl-1-{[(3-fluorobenzyl)amino]carbonyl}-1H-pyrazol-3-yl)-4- |
| | hydroxyphenyl]isonicotinamide |
| 2380 | N-(3-{5-cyclobutyl-1-[(cyclopropylamino)carbonyl]-1H-pyrazol-3-yl}-4- |
| | hydroxyphenyl)isonicotinamide |
| 2381 | N-(3-{5-cyclopentyl-1-[(isopropylamino)carbonyl]-1H-pyrazol-3-yl}-4- |
| | hydroxyphenyl)isonicotinamide |
| 2382 | N-(3-{5-cyclopentyl-1-[(ethylamino)carbonyl]-1H-pyrazol-3-yl}-4- |
| | hydroxyphenyl)isonicotinamide |
| 2383 | N-(3-{1-[(allylamino)carbonyl]-5-cyclopentyl-1H-pyrazol-3-yl}-4- |
| | hydroxyphenyl)isonicotinamide |
| 2384 | N-(3-{5-cyclopentyl-1-[(propylamino)carbonyl]-1H-pyrazol-3-yl}-4- |
| | hydroxyphenyl)isonicotinamide |
| 2385 | N-(3-{1-[(benzylamino)carbonyl]-5-cyclopentyl-1H-pyrazol-3-yl}-4- |
| | hydroxyphenyl)isonicotinamide |
| 2386 | N-[3-(5-cyclopentyl-1-{[(2-phenylethyl)amino]carbonyl}-1H-pyrazol-3-yl)-4- |
| | hydroxyphenyl]isonicotinamide |
| 2387 | N-[3-(5-cyclopentyl-1-{[(2-methylbenzyl)amino]carbonyl}-1H-pyrazol-3-yl)-4- |
| | hydroxyphenyl]isonicotinamide |
| 2388 | N-[3-(5-cyclopentyl-1-{[(3-methylbenzyl)amino]carbonyl}-1H-pyrazol-3-yl)-4- |
| | hydroxyphenyl]isonicotinamide |
| 2389 | N-[3-(5-cyclopentyl-1-{[(4-methylbenzyl)amino]carbonyl}-1H-pyrazol-3-yl)-4- |
| | hydroxyphenyl]isonicotinamide |
| 2390 | N-[3-(1-{[(2-chlorobenzyl)amino]carbonyl}-5-cyclopentyl-1H-pyrazol-3-yl)-4- |
| | hydroxyphenyl]isonicotinamide |
| 2391 | N-[3-(5-cyclopentyl-1-{[(4-fluorobenzyl)amino]carbonyl}-1H-pyrazol-3-yl)-4- |
| | hydroxyphenyl]isonicotinamide |
| 2392 | N-[3-(5-cyclopentyl-1-{[(3,4-dichlorobenzyl)amino]carbonyl}-1H-pyrazol-3-yl)- |
| | 4-hydroxyphenyl]isonicotinamide |
| 2393 | N-[3-(5-cyclopentyl-1-{[(4-methoxybenzyl)amino]carbonyl}-1H-pyrazol-3-yl)-4- |
| | hydroxyphenyl]isonicotinamide |
| 2394 | N-(3-{1-[(sec-butylamino)carbonyl]-5-cyclopentyl-1H-pyrazol-3-yl}-4- |
| | hydroxyphenyl)isonicotinamide |
| 2395 | N-[3-(5-cyclopentyl-1-{[(2-fluorobenzyl)amino]carbonyl}-1H-pyrazol-3-yl)-4- |
| | hydroxyphenyl]isonicotinamide |
| 2396 | N-[3-(5-cyclopentyl-1-{[(3-fluorobenzyl)amino]carbonyl}-1H-pyrazol-3-yl)-4- |
| | hydroxyphenyl]isonicotinamide |
| 2397 | N-(3-{5-cyclopentyl-1-[(cyclopropylamino)carbonyl]-1H-pyrazol-3-yl}-4- |
| | hydroxyphenyl)isonicotinamide |
| 2398 | 3-{5-[(1,3-benzodioxol-5-ylcarbonyl)amino]-2-hydroxyphenyl}-5-cyclopropyl-N- |
| | isopropyl-1H-pyrazole-1-carboxamide |
| 2399 | 3-{5-[(1,3-benzodioxol-5-ylcarbonyl)amino]-2-hydroxyphenyl}-5-cyclopropyl-N- |
| | ethyl-1H-pyrazole-1-carboxamide |
| 2400 | N-allyl-3-{5-[(1,3-benzodioxol-5-ylcarbonyl)amino]-2-hydroxyphenyl}-5- |
| | cyclopropyl-1H-pyrazole-1-carboxamide |
| 2401 | 3-{5-[(1,3-benzodioxol-5-ylcarbonyl)amino]-2-hydroxyphenyl}-5-cyclopropyl-N- |
| | propyl-1H-pyrazole-1-carboxamide |
| 2402 | 3-{5-[(1,3-benzodioxol-5-ylcarbonyl)amino]-2-hydroxyphenyl}-N-benzyl-5- |
| | cyclopropyl-1H-pyrazole-1-carboxamide |
| 2403 | 3-{5-[(1,3-benzodioxol-5-ylcarbonyl)amino]-2-hydroxyphenyl}-5-cyclopropyl-N- |
| | (2-phenylethyl)-1H-pyrazole-1-carboxamide |
| 2404 | 3-{5-[(1,3-benzodioxol-5-ylcarbonyl)amino]-2-hydroxyphenyl}-5-cyclopropyl-N- |
| | (2-methylbenzyl)-1H-pyrazole-1-carboxamide |
| 2405 | 3-{5-[(1,3-benzodioxol-5-ylcarbonyl)amino]-2-hydroxyphenyl}-5-cyclopropyl-N- |
| | (3-methylbenzyl)-1H-pyrazole-1-carboxamide |
| 2406 | 3-{5-[(1,3-benzodioxol-5-ylcarbonyl)amino]-2-hydroxyphenyl}-5-cyclopropyl-N- |
| | (4-methylbenzyl)-1H-pyrazole-1-carboxamide |
| 2407 | 3-{5-[(1,3-benzodioxol-5-ylcarbonyl)amino]-2-hydroxyphenyl}-N-(2- |
| | chlorobenzyl)-5-cyclopropyl-1H-pyrazole-1-carboxamide |
| 2408 | 3-{5-[(1,3-benzodioxol-5-ylcarbonyl)amino]-2-hydroxyphenyl}-5-cyclopropyl-N- |
| | (4-fluorobenzyl)-1H-pyrazole-1-carboxamide |
| 2409 | 3-{5-[(1,3-benzodioxol-5-ylcarbonyl)amino]-2-hydroxyphenyl}-5-cyclopropyl-N- |
| | (3,4-dichlorobenzyl)-1H-pyrazole-1-carboxamide |
| 2410 | 3-{5-[(1,3-benzodioxol-5-ylcarbonyl)amino]-2-hydroxyphenyl}-5-cyclopropyl-N- |
| | (4-methoxybenzyl)-1H-pyrazole-1-carboxamide |
| 2411 | 3-{5-[(1,3-benzodioxol-5-ylcarbonyl)amino]-2-hydroxyphenyl}-N-(sec-butyl)-5- |
| | cyclopropyl-1H-pyrazole-1-carboxamide |
| 2412 | 3-{5-[(1,3-benzodioxol-5-ylcarbonyl)amino]-2-hydroxyphenyl}-5-cyclopropyl-N- |
| | (2-fluorobenzyl)-1H-pyrazole-1-carboxamide |
| 2413 | 3-{5-[(1,3-benzodioxol-5-ylcarbonyl)amino]-2-hydroxyphenyl}-5-cyclopropyl-N- |
| | (3-fluorobenzyl)-1H-pyrazole-1-carboxamide |
| 2414 | 3-{5-[(1,3-benzodioxol-5-ylcarbonyl)amino]-2-hydroxyphenyl}-N,5- |
| | dicyclopropyl-1H-pyrazole-1-carboxamide |
| 2415 | 3-{5-[(1,3-benzodioxol-5-ylcarbonyl)amino]-2-hydroxyphenyl}-5-cyclobutyl-N- |
| | isopropyl-1H-pyrazole-1-carboxamide |
| 2416 | 3-{5-[(1,3-benzodioxol-5-ylcarbonyl)amino]-2-hydroxyphenyl}-5-cyclobutyl-N- |
| | ethyl-1H-pyrazole-1-carboxamide |
| 2417 | N-allyl-3-{5-[(1,3-benzodioxol-5-ylcarbonyl)amino]-2-hydroxyphenyl}-5- |
| | cyclobutyl-1H-pyrazole-1-carboxamide |
| 2418 | 3-{5-[(1,3-benzodioxol-5-ylcarbonyl)amino]-2-hydroxyphenyl}-5-cyclobutyl-N- |
| | propyl-1H-pyrazole-1-carboxamide |
| 2419 | 3-{5-[(1,3-benzodioxol-5-ylcarbonyl)amino]-2-hydroxyphenyl}-N-benzyl-5- |
| | cyclobutyl-1H-pyrazole-1-carboxamide |
| 2420 | 3-{5-[(1,3-benzodioxol-5-ylcarbonyl)amino]-2-hydroxyphenyl}-5-cyclobutyl-N- |
| | (2-phenylethyl)-1H-pyrazole-1-carboxamide |
| 2421 | 3-{5-[(1,3-benzodioxol-5-ylcarbonyl)amino]-2-hydroxyphenyl}-5-cyclobutyl-N- |
| | (2-methylbenzyl)-1H-pyrazole-1-carboxamide |
| 2422 | 3-{5-[(1,3-benzodioxol-5-ylcarbonyl)amino]-2-hydroxyphenyl}-5-cyclobutyl-N- |
| | (3-methylbenzyl)-1H-pyrazole-1-carboxamide |
| 2423 | 3-{5-[(1,3-benzodioxol-5-ylcarbonyl)amino]-2-hydroxyphenyl}-5-cyclobutyl-N- |
| | (4-methylbenzyl)-1H-pyrazole-1-carboxamide |
| 2424 | 3-{5-[(1,3-benzodioxol-5-ylcarbonyl)amino]-2-hydroxyphenyl}-N-(2- |
| | chlorobenzyl)-5-cyclobutyl-1H-pyrazole-1-carboxamide |
| 2425 | 3-{5-[(1,3-benzodioxol-5-ylcarbonyl)amino]-2-hydroxyphenyl}-5-cyclobutyl-N- |
| | (4-fluorobenzyl)-1H-pyrazole-1-carboxamide |
| 2426 | 3-{5-[(1,3-benzodioxol-5-ylcarbonyl)amino]-2-hydroxyphenyl}-5-cyclobutyl-N- |
| | (3,4-dichlorobenzyl)-1H-pyrazole-1-carboxamide |
| 2427 | 3-{5-[(1,3-benzodioxol-5-ylcarbonyl)amino]-2-hydroxyphenyl}-5-cyclobutyl-N- |
| | (4-methoxybenzyl)-1H-pyrazole-1-carboxamide |
| 2428 | 3-{5-[(1,3-benzodioxol-5-ylcarbonyl)amino]-2-hydroxyphenyl}-N-(sec-butyl)-5- |
| | cyclobutyl-1H-pyrazole-1-carboxamide |
| 2429 | 3-{5-[(1,3-benzodioxol-5-ylcarbonyl)amino]-2-hydroxyphenyl}-5-cyclobutyl-N- |
| | (2-fluorobenzyl)-1H-pyrazole-1-carboxamide |
| 2430 | 3-{5-[(1,3-benzodioxol-5-ylcarbonyl)amino]-2-hydroxyphenyl}-5-cyclobutyl-N- |
| | (3-fluorobenzyl)-1H-pyrazole-1-carboxamide |
| 2431 | 3-{5-[(1,3-benzodioxol-5-ylcarbonyl)amino]-2-hydroxyphenyl}-5-cyclobutyl-N- |
| | cyclopropyl-1H-pyrazole-1-carboxamide |
| 2432 | 3-{5-[(1,3-benzodioxol-5-ylcarbonyl)amino]-2-hydroxyphenyl}-5-cyclopentyl-N- |
| | isopropyl-1H-pyrazole-1-carboxamide |
| 2433 | 3-{5-[(1,3-benzodioxol-5-ylcarbonyl)amino]-2-hydroxyphenyl}-5-cyclopentyl-N- |
| | ethyl-1H-pyrazole-1-carboxamide |
| 2434 | N-allyl-3-{5-[(1,3-benzodioxol-5-ylcarbonyl)amino]-2-hydroxyphenyl}-5- |
| | cyclopentyl-1H-pyrazole-1-carboxamide |
| 2435 | 3-{5-[(1,3-benzodioxol-5-ylcarbonyl)amino]-2-hydroxyphenyl}-5-cyclopentyl-N- |
| | propyl-1H-pyrazole-1-carboxamide |
| 2436 | 3-{5-[(1,3-benzodioxol-5-ylcarbonyl)amino]-2-hydroxyphenyl}-N-benzyl-5- |
| | cyclopentyl-1H-pyrazole-1-carboxamide |
| 2437 | 3-{5-[(1,3-benzodioxol-5-ylcarbonyl)amino]-2-hydroxyphenyl}-5-cyclopentyl-N- |
| | (2-phenylethyl)-1H-pyrazole-1-carboxamide |
| 2438 | 3-{5-[(1,3-benzodioxol-5-ylcarbonyl)amino]-2-hydroxyphenyl}-5-cyclopentyl-N- |
| | (2-methylbenzyl)-1H-pyrazole-1-carboxamide |
| 2439 | 3-{5-[(1,3-benzodioxol-5-ylcarbonyl)amino]-2-hydroxyphenyl}-5-cyclopentyl-N- |
| | (3-methylbenzyl)-1H-pyrazole-1-carboxamide |
| 2440 | 3-{5-[(1,3-benzodioxol-5-ylcarbonyl)amino]-2-hydroxyphenyl}-5-cyclopentyl-N- |
| | (4-methylbenzyl)-1H-pyrazole-1-carboxamide |
| 2441 | 3-{5-[(1,3-benzodioxol-5-ylcarbonyl)amino]-2-hydroxyphenyl}-N-(2- |
| | chlorobenzyl)-5-cyclopentyl-1H-pyrazole-1-carboxamide |
| 2442 | 3-{5-[(1,3-benzodioxol-5-ylcarbonyl)amino]-2-hydroxyphenyl}-5-cyclopentyl-N- |
| | (4-fluorobenzyl)-1H-pyrazole-1-carboxamide |
| 2443 | 3-{5-[(1,3-benzodioxol-5-ylcarbonyl)amino]-2-hydroxyphenyl}-5-cyclopentyl-N- |
| | (3,4-dichlorobenzyl)-1H-pyrazole-1-carboxamide |
| 2444 | 3-{5-[(1,3-benzodioxol-5-ylcarbonyl)amino]-2-hydroxyphenyl}-5-cyclopentyl-N- |
| | (4-methoxybenzyl)-1H-pyrazole-1-carboxamide |
| 2445 | 3-{5-[(1,3-benzodioxol-5-ylcarbonyl)amino]-2-hydroxyphenyl}-N-(sec-butyl)-5- |
| | cyclopentyl-1H-pyrazole-1-carboxamide |
| 2446 | 3-{5-[(1,3-benzodioxol-5-ylcarbonyl)amino]-2-hydroxyphenyl}-5-cyclopentyl-N- |
| | (2-fluorobenzyl)-1H-pyrazole-1-carboxamide |
| 2447 | 3-{5-[(1,3-benzodioxol-5-ylcarbonyl)amino]-2-hydroxyphenyl}-5-cyclopentyl-N- |
| | (3-fluorobenzyl)-1H-pyrazole-1-carboxamide |
| 2448 | 3-{5-[(1,3-benzodioxol-5-ylcarbonyl)amino]-2-hydroxyphenyl}-5-cyclopentyl-N- |
| | cyclopropyl-1H-pyrazole-1-carboxamide |
| 2449 | 5-cyclopropyl-3-{5-[(2-ethylbutanoyl)amino]-2-hydroxyphenyl}-N-isopropyl-1H- |
| | pyrazole-1-carboxamide |
| 2450 | 5-cyclopropyl-N-ethyl-3-{5-[(2-ethylbutanoyl)amino]-2-hydroxyphenyl}-1H- |
| | pyrazole-1-carboxamide |
| 2451 | N-allyl-5-cyclopropyl-3-{5-[(2-ethylbutanoyl)amino]-2-hydroxyphenyl}-1H- |
| | pyrazole-1-carboxamide |
| 2452 | 5-cyclopropyl-3-{5-[(2-ethylbutanoyl)amino]-2-hydroxyphenyl}-N-propyl-1H- |
| | pyrazole-1-carboxamide |
| 2453 | N-benzyl-5-cyclopropyl-3-{5-[(2-ethylbutanoyl)amino]-2-hydroxyphenyl}-1H- |
| | pyrazole-1-carboxamide |
| 2454 | 5-cyclopropyl-3-{5-[(2-ethylbutanoyl)amino]-2-hydroxyphenyl}-N-(2- |
| | phenylethyl)-1H-pyrazole-1-carboxamide |
| 2455 | 5-cyclopropyl-3-{5-[(2-ethylbutanoyl)amino]-2-hydroxyphenyl}-N-(2- |
| | methylbenzyl)-1H-pyrazole-1-carboxamide |
| 2456 | 5-cyclopropyl-3-{5-[(2-ethylbutanoyl)amino]-2-hydroxyphenyl}-N-(3- |
| | methylbenzyl)-1H-pyrazole-1-carboxamide |
| 2457 | 5-cyclopropyl-3-{5-[(2-ethylbutanoyl)amino]-2-hydroxyphenyl}-N-(4- |
| | methylbenzyl)-1H-pyrazole-1-carboxamide |
| 2458 | N-(2-chlorobenzyl)-5-cyclopropyl-3-{5-[(2-ethylbutanoyl)amino]-2- |
| | hydroxyphenyl}-1H-pyrazole-1-carboxamide |
| 2459 | 5-cyclopropyl-3-{5-[(2-ethylbutanoyl)amino]-2-hydroxyphenyl}-N-(4- |
| | fluorobenzyl)-1H-pyrazole-1-carboxamide |
| 2460 | 5-cyclopropyl-N-(3,4-dichlorobenzyl)-3-{5-[(2-ethylbutanoyl)amino]-2- |
| | hydroxyphenyl}-1H-pyrazole-1-carboxamide |
| 2461 | 5-cyclopropyl-3-{5-[(2-ethylbutanoyl)amino]-2-hydroxyphenyl}-N-(4- |
| | methoxybenzyl)-1H-pyrazole-1-carboxamide |
| 2462 | N-(sec-butyl)-5-cyclopropyl-3-{5-[(2-ethylbutanoyl)amino]-2-hydroxyphenyl}- |
| | 1H-pyrazole-1-carboxamide |
| 2463 | 5-cyclopropyl-3-{5-[(2-ethylbutanoyl)amino]-2-hydroxyphenyl}-N-(2- |
| | fluorobenzyl)-1H-pyrazole-1-carboxamide |
| 2464 | 5-cyclopropyl-3-{5-[(2-ethylbutanoyl)amino]-2-hydroxyphenyl}-N-(3- |
| | fluorobenzyl)-1H-pyrazole-1-carboxamide |
| 2465 | N,5-dicyclopropyl-3-{5-[(2-ethylbutanoyl)amino]-2-hydroxyphenyl}-1H- |
| | pyrazole-1-carboxamide |
| 2466 | 5-cyclobutyl-3-{5-[(2-ethylbutanoyl)amino]-2-hydroxyphenyl}-N-isopropyl-1H- |
| | pyrazole-1-carboxamide |
| 2467 | 5-cyclobutyl-N-ethyl-3-{5-[(2-ethylbutanoyl)amino]-2-hydroxyphenyl}-1H- |
| | pyrazole-1-carboxamide |
| 2468 | N-allyl-5-cyclobutyl-3-{5-[(2-ethylbutanoyl)amino]-2-hydroxyphenyl}-1H- |
| | pyrazole-1-carboxamide |
| 2469 | 5-cyclobutyl-3-{5-[(2-ethylbutanoyl)amino]-2-hydroxyphenyl}-N-propyl-1H- |
| | pyrazole-1-carboxamide |
| 2470 | N-benzyl-5-cyclobutyl-3-{5-[(2-ethylbutanoyl)amino]-2-hydroxyphenyl}-1H- |
| | pyrazole-1-carboxamide |
| 2471 | 5-cyclobutyl-3-{5-[(2-ethylbutanoyl)amino]-2-hydroxyphenyl}-N-(2-phenylethyl)- |
| | 1H-pyrazole-1-carboxamide |
| 2472 | 5-cyclobutyl-3-{5-[(2-ethylbutanoyl)amino]-2-hydroxyphenyl}-N-(2- |
| | methylbenzyl)-1H-pyrazole-1-carboxamide |
| 2473 | 5-cyclobutyl-3-{5-[(2-ethylbutanoyl)amino]-2-hydroxyphenyl}-N-(3- |
| | methylbenzyl)-1H-pyrazole-1-carboxamide |
| 2474 | 5-cyclobutyl-3-{5-[(2-ethylbutanoyl)amino]-2-hydroxyphenyl}-N-(4- |
| | methylbenzyl)-1H-pyrazole-1-carboxamide |
| 2475 | N-(2-chlorobenzyl)-5-cyclobutyl-3-{5-[(2-ethylbutanoyl)amino]-2- |
| | hydroxyphenyl}-1H-pyrazole-1-carboxamide |
| 2476 | 5-cyclobutyl-3-{5-[(2-ethylbutanoyl)amino]-2-hydroxyphenyl}-N-(4- |
| | fluorobenzyl)-1H-pyrazole-1-carboxamide |
| 2477 | 5-cyclobutyl-N-(3,4-dichlorobenzyl)-3-{5-[(2-ethylbutanoyl)amino]-2- |
| | hydroxyphenyl}-1H-pyrazole-1-carboxamide |
| 2478 | 5-cyclobutyl-3-{5-[(2-ethylbutanoyl)amino]-2-hydroxyphenyl}-N-(4- |
| | methoxybenzyl)-1H-pyrazole-1-carboxamide |
| 2479 | N-(sec-butyl)-5-cyclobutyl-3-{5-[(2-ethylbutanoyl)amino]-2-hydroxyphenyl}-1H- |
| | pyrazole-1-carboxamide |
| 2480 | 5-cyclobutyl-3-{5-[(2-ethylbutanoyl)amino]-2-hydroxyphenyl}-N-(2- |
| | fluorobenzyl)-1H-pyrazole-1-carboxamide |
| 2481 | 5-cyclobutyl-3-{5-[(2-ethylbutanoyl)amino]-2-hydroxyphenyl}-N-(3- |
| | fluorobenzyl)-1H-pyrazole-1-carboxamide |
| 2482 | 5-cyclobutyl-N-cyclopropyl-3-{5-[(2-ethylbutanoyl)amino]-2-hydroxyphenyl}- |
| | 1H-pyrazole-1-carboxamide |
| 2483 | 5-cyclopentyl-3-{5-[(2-ethylbutanoyl)amino]-2-hydroxyphenyl}-N-isopropyl-1H- |
| | pyrazole-1-carboxamide |
| 2484 | 5-cyclopentyl-N-ethyl-3-{5-[(2-ethylbutanoyl)amino]-2-hydroxyphenyl}-1H- |
| | pyrazole-1-carboxamide |
| 2485 | N-allyl-5-cyclopentyl-3-{5-[(2-ethylbutanoyl)amino]-2-hydroxyphenyl}-1H- |
| | pyrazole-1-carboxamide |
| 2486 | 5-cyclopentyl-3-{5-[(2-ethylbutanoyl)amino]-2-hydroxyphenyl}-N-propyl-1H- |
| | pyrazole-1-carboxamide |
| 2487 | N-benzyl-5-cyclopentyl-3-{5-[(2-ethylbutanoyl)amino]-2-hydroxyphenyl}-1H- |
| | pyrazole-1-carboxamide |
| 2488 | 5-cyclopentyl-3-{5-[(2-ethylbutanoyl)amino]-2-hydroxyphenyl}-N-(2- |
| | phenylethyl)-1H-pyrazole-1-carboxamide |
| 2489 | 5-cyclopentyl-3-{5-[(2-ethylbutanoyl)amino]-2-hydroxyphenyl}-N-(2- |
| | methylbenzyl)-1H-pyrazole-1-carboxamide |
| 2490 | 5-cyclopentyl-3-{5-[(2-ethylbutanoyl)amino]-2-hydroxyphenyl}-N-(3- |
| | methylbenzyl)-1H-pyrazole-1-carboxamide |
| 2491 | 5-cyclopentyl-3-{5-[(2-ethylbutanoyl)amino]-2-hydroxyphenyl}-N-(4- |
| | methylbenzyl)-1H-pyrazole-1-carboxamide |
| 2492 | 5-cyclopentyl-3-{5-[(2-ethylbutanoyl)amino]-2-hydroxyphenyl}-N-(4- |
| | fluorobenzyl)-1H-pyrazole-1-carboxamide |
| 2493 | 5-cyclopentyl-3-{5-[(2-ethylbutanoyl)amino]-2-hydroxyphenyl}-N-(4- |
| | fluorobenzyl)-1H-pyrazole-1-carboxamide |
| 2494 | 5-cyclopentyl-N-(3,4-dichlorobenzyl)-3-{5-[(2-ethylbutanoyl)amino]-2- |
| | hydroxyphenyl}-1H-pyrazole-1-carboxamide |
| 2495 | 5-cyclopentyl-3-{5-[(2-ethylbutanoyl)amino]-2-hydroxyphenyl}-N-(4- |
| | methoxybenzyl)-1H-pyrazole-1-carboxamide |
| 2496 | N-(sec-butyl)-5-cyclopentyl-3-{5-[(2-ethylbutanoyl)amino]-2-hydroxyphenyl}- |
| | 1H-pyrazole-1-carboxamide |
| 2497 | 5-cyclopentyl-3-{5-[(2-ethylbutanoyl)amino]-2-hydroxyphenyl}-N-(2- |
| | fluorobenzyl)-1H-pyrazole-1-carboxamide |
| 2498 | 5-cyclopentyl-3-{5-[(2-ethylbutanoyl)amino]-2-hydroxyphenyl}-N-(3- |
| | fluorobenzyl)-1H-pyrazole-1-carboxamide |
| 2499 | 5-cyclopentyl-N-cyclopropyl-3-{5-[(2-ethylbutanoyl)amino]-2-hydroxyphenyl}- |
| | 1H-pyrazole-1-carboxamide |
| 2500 | 3-{5-[(1-benzothien-2-ylcarbonyl)amino]-2-hydroxyphenyl}-5-cyclopropyl-N- |
| | isopropyl-1H-pyrazole-1-carboxamide |
| 2501 | 3-{5-[(1-benzothien-2-ylcarbonyl)amino]-2-hydroxyphenyl}-5-cyclopropyl-N- |
| | ethyl-1H-pyrazole-1-carboxamide |
| 2502 | N-allyl-3-{5-[(1-benzothien-2-ylcarbonyl)amino]-2-hydroxyphenyl}-5- |
| | cyclopropyl-1H-pyrazole-1-carboxamide |
| 2503 | 3-{5-[(1-benzothien-2-ylcarbonyl)amino]-2-hydroxyphenyl}-5-cyclopropyl-N- |
| | propyl-1H-pyrazole-1-carboxamide |
| 2504 | 3-{5-[(1-benzothien-2-ylcarbonyl)amino]-2-hydroxyphenyl}-N-benzyl-5- |
| | cyclopropyl-1H-pyrazole-1-carboxamide |
| 2505 | 3-{5-[(1-benzothien-2-ylcarbonyl)amino]-2-hydroxyphenyl}-5-cyclopropyl-N-(2- |
| | phenylethyl)-1H-pyrazole-1-carboxamide |
| 2506 | 3-{5-[(1-benzothien-2-ylcarbonyl)amino]-2-hydroxyphenyl}-5-cyclopropyl-N-(2- |
| | methylbenzyl)-1H-pyrazole-1-carboxamide |
| 2507 | 3-{5-[(1-benzothien-2-ylcarbonyl)amino]-2-hydroxyphenyl}-5-cyclopropyl-N-(3- |
| | methylbenzyl)-1H-pyrazole-1-carboxamide |
| 2508 | 3-{5-[(1-benzothien-2-ylcarbonyl)amino]-2-hydroxyphenyl}-5-cyclopropyl-N-(4- |
| | methylbenzyl)-1H-pyrazole-1-carboxamide |
| 2509 | 3-{5-[(1-benzothien-2-ylcarbonyl)amino]-2-hydroxyphenyl}-N-(2-chlorobenzyl)- |
| | 5-cyclopropyl-1H-pyrazole-1-carboxamide |
| 2510 | 3-{5-[(1-benzothien-2-ylcarbonyl)amino]-2-hydroxyphenyl}-5-cyclopropyl-N-(4- |
| | fluorobenzyl)-1H-pyrazole-1-carboxamide |
| 2511 | 3-{5-[(1-benzothien-2-ylcarbonyl)amino]-2-hydroxyphenyl}-5-cyclopropyl-N- |
| | (3,4-dichlorobenzyl)-1H-pyrazole-1-carboxamide |
| 2512 | 3-{5-[(1-benzothien-2-ylcarbonyl)amino]-2-hydroxyphenyl}-5-cyclopropyl-N-(4- |
| | methoxybenzyl)-1H-pyrazole-1-carboxamide |
| 2513 | 3-{5-[(1-benzothien-2-ylcarbonyl)amino]-2-hydroxyphenyl}-N-(sec-butyl)-5- |
| | cyclopropyl-1H-pyrazole-1-carboxamide |
| 2514 | 3-{5-[(1-benzothien-2-ylcarbonyl)amino]-2-hydroxyphenyl}-5-cyclopropyl-N-(2- |
| | fluorobenzyl)-1H-pyrazole-1-carboxamide |
| 2515 | 3-{5-[(1-benzothien-2-ylcarbonyl)amino]-2-hydroxyphenyl}-5-cyclopropyl-N-(3- |
| | fluorobenzyl)-1H-pyrazole-1-carboxamide |
| 2516 | 3-{5-[(1-benzothien-2-ylcarbonyl)amino]-2-hydroxyphenyl}-N,5-dicyclopropyl- |
| | 1H-pyrazole-1-carboxamide |
| 2517 | 3-{5-[(1-benzothien-2-ylcarbonyl)amino]-2-hydroxyphenyl}-5-cyclobutyl-N- |
| | isopropyl-1H-pyrazole-1-carboxamide |
| 2518 | 3-{5-[(1-benzothien-2-ylcarbonyl)amino]-2-hydroxyphenyl}-5-cyclobutyl-N- |
| | ethyl-1H-pyrazole-1-carboxamide |
| 2519 | N-allyl-3-{5-[(1-benzothien-2-ylcarbonyl)amino]-2-hydroxyphenyl}-5-cyclobutyl- |
| | 1H-pyrazole-1-carboxamide |
| 2520 | 3-{5-[(1-benzothien-2-ylcarbonyl)amino]-2-hydroxyphenyl}-5-cyclobutyl-N- |
| | propyl-1H-pyrazole-1-carboxamide |
| 2521 | 3-{5-[(1-benzothien-2-ylcarbonyl)amino]-2-hydroxyphenyl}-N-benzyl-5- |
| | cyclobutyl-1H-pyrazole-1-carboxamide |
| 2522 | 3-{5-[(1-benzothien-2-ylcarbonyl)amino]-2-hydroxyphenyl}-5-cyclobutyl-N-(2- |
| | phenylethyl)-1H-pyrazole-1-carboxamide |
| 2523 | 3-{5-[(1-benzothien-2-ylcarbonyl)amino]-2-hydroxyphenyl}-5-cyclobutyl-N-(2- |
| | methylbenzyl)-1H-pyrazole-1-carboxamide |
| 2524 | 3-{5-[(1-benzothien-2-ylcarbonyl)amino]-2-hydroxyphenyl}-5-cyclobutyl-N-(3- |
| | methylbenzyl)-1H-pyrazole-1-carboxamide |
| 2525 | 3-{5-[(1-benzothien-2-ylcarbonyl)amino]-2-hydroxyphenyl}-5-cyclobutyl-N-(4- |
| | methylbenzyl)-1H-pyrazole-1-carboxamide |
| 2526 | 3-{5-[(1-benzothien-2-ylcarbonyl)amino]-2-hydroxyphenyl}-N-(2-chlorobenzyl)- |
| | 5-cyclobutyl-1H-pyrazole-1-carboxamide |
| 2527 | 3-{5-[(1-benzothien-2-ylcarbonyl)amino]-2-hydroxyphenyl}-5-cyclobutyl-N-(4- |
| | fluorobenzyl)-1H-pyrazole-1-carboxamide |
| 2528 | 3-{5-[(1-benzothien-2-ylcarbonyl)amino]-2-hydroxyphenyl}-5-cyclobutyl-N-(3,4- |
| | dichlorobenzyl)-1H-pyrazole-1-carboxamide |
| 2529 | 3-{5-[(1-benzothien-2-ylcarbonyl)amino]-2-hydroxyphenyl}-5-cyclobutyl-N-(4- |
| | methoxybenzyl)-1H-pyrazole-1-carboxamide |
| 2530 | 3-{5-[(1-benzothien-2-ylcarbonyl)amino]-2-hydroxyphenyl}-N-(sec-butyl)-5- |
| | cyclobutyl-1H-pyrazole-1-carboxamide |
| 2531 | 3-{5-[(1-benzothien-2-ylcarbonyl)amino]-2-hydroxyphenyl}-5-cyclobutyl-N-(2- |
| | fluorobenzyl)-1H-pyrazole-1-carboxamide |
| 2532 | 3-{5-[(1-benzothien-2-ylcarbonyl)amino]-2-hydroxyphenyl}-5-cyclobutyl-N-(3- |
| | fluorobenzyl)-1H-pyrazole-1-carboxamide |
| 2533 | 3-{5-[(1-benzothien-2-ylcarbonyl)amino]-2-hydroxyphenyl}-5-cyclobutyl-N- |
| | cyclopropyl-1H-pyrazole-1-carboxamide |
| 2534 | 3-{5-[(1-benzothien-2-ylcarbonyl)amino]-2-hydroxyphenyl}-5-cyclopentyl-N- |
| | isopropyl-1H-pyrazole-1-carboxamide |
| 2535 | 3-{5-[(1-benzothien-2-ylcarbonyl)amino]-2-hydroxyphenyl}-5-cyclopentyl-N- |
| | ethyl-1H-pyrazole-1-carboxamide |
| 2536 | N-allyl-3-{5-[(1-benzothien-2-ylcarbonyl)amino]-2-hydroxyphenyl}-5- |
| | cyclopentyl-1H-pyrazole-1-carboxamide |
| 2537 | 3-{5-[(1-benzothien-2-ylcarbonyl)amino]-2-hydroxyphenyl}-5-cyclopentyl-N- |
| | propyl-1H-pyrazole-1-carboxamide |
| 2538 | 3-{5-[(1-benzothien-2-ylcarbonyl)amino]-2-hydroxyphenyl}-N-benzyl-5- |
| | cyclopentyl-1H-pyrazole-1-carboxamide |
| 2539 | 3-{5-[(1-benzothien-2-ylcarbonyl)amino]-2-hydroxyphenyl}-5-cyclopentyl-N-(2- |
| | phenylethyl)-1H-pyrazole-1-carboxamide |
| 2540 | 3-{5-[(1-benzothien-2-ylcarbonyl)amino]-2-hydroxyphenyl}-5-cyclopentyl-N-(2- |
| | methylbenzyl)-1H-pyrazole-1-carboxamide |
| 2541 | 3-{5-[(1-benzothien-2-ylcarbonyl)amino]-2-hydroxyphenyl}-5-cyclopentyl-N-(3- |
| | methylbenzyl)-1H-pyrazole-1-carboxamide |
| 2542 | 3-{5-[(1-benzothien-2-ylcarbonyl)amino]-2-hydroxyphenyl}-5-cyclopentyl-N-(4- |
| | methylbenzyl)-1H-pyrazole-1-carboxamide |
| 2543 | 3-{5-[(1-benzothien-2-ylcarbonyl)amino]-2-hydroxyphenyl}-N-(2-chlorobenzyl)- |
| | 5-cyclopentyl-1H-pyrazole-1-carboxamide |
| 2544 | 3-{5-[(1-benzothien-2-ylcarbonyl)amino]-2-hydroxyphenyl}-5-cyclopentyl-N-(4- |
| | fluorobenzyl)-1H-pyrazole-1-carboxamide |
| 2545 | 3-{5-[(1-benzothien-2-ylcarbonyl)amino]-2-hydroxyphenyl}-5-cyclopentyl-N- |
| | (3,4-dichlorobenzyl)-1H-pyrazole-1-carboxamide |
| 2546 | 3-{5-[(1-benzothien-2-ylcarbonyl)amino]-2-hydroxyphenyl}-5-cyclopentyl-N-(4- |
| | methoxybenzyl)-1H-pyrazole-1-carboxamide |
| 2547 | 3-{5-[(1-benzothien-2-ylcarbonyl)amino]-2-hydroxyphenyl}-N-(sec-butyl)-5- |
| | cyclopentyl-1H-pyrazole-1-carboxamide |
| 2548 | 3-{5-[(1-benzothien-2-ylcarbonyl)amino]-2-hydroxyphenyl}-5-cyclopentyl-N-(2- |
| | fluorobenzyl)-1H-pyrazole-1-carboxamide |
| 2549 | 3-{5-[(1-benzothien-2-ylcarbonyl)amino]-2-hydroxyphenyl}-5-cyclopentyl-N-(3- |
| | fluorobenzyl)-1H-pyrazole-1-carboxamide |
| 2550 | 3-{5-[(1-benzothien-2-ylcarbonyl)amino]-2-hydroxyphenyl}-5-cyclopentyl-N- |
| | cyclopropyl-1H-pyrazole-1-carboxamide |
| 2551 | 2-chloro-N-(3-{5-cyclopropyl-1-[(isopropylamino)carbonyl]-1H-pyrazol-3-yl}-4- |
| | hydroxyphenyl)nicotinamide |
| 2552 | 2-chloro-N-(3-{5-cyclopropyl-1-[(ethylamino)carbonyl]-1H-pyrazol-3-yl}-4- |
| | hydroxyphenyl)nicotinamide |
| 2553 | N-(3-{1-[(allylamino)carbonyl]-5-cyclopropyl-1H-pyrazol-3-yl}-4- |
| | hydroxyphenyl)-2-chloronicotinamide |
| 2554 | 2-chloro-N-(3-{5-cyclopropyl-1-[(propylamino)carbonyl]-1H-pyrazol-3-yl}-4- |
| | hydroxyphenyl)nicotinamide |
| 2555 | N-(3-{1-[(benzylamino)carbonyl]-5-cyclopropyl-1H-pyrazol-3-yl}-4- |
| | hydroxyphenyl)-2-chloronicotinamide |
| 2556 | 2-chloro-N-[3-(5-cyclopropyl-1-{[(2-phenylethyl)amino]carbonyl}-1H-pyrazol-3- |
| | yl)-4-hydroxyphenyl]nicotinamide |
| 2557 | 2-chloro-N-[3-(5-cyclopropyl-1-{[(2-methylbenzyl)amino]carbonyl}-1H-pyrazol- |
| | 3-yl)-4-hydroxyphenyl]nicotinamide |
| 2558 | 2-chloro-N-[3-(5-cyclopropyl-1-{[(3-methylbenzyl)amino]carbonyl}-1H-pyrazol- |
| | 3-yl)-4-hydroxyphenyl]nicotinamide |
| 2559 | 2-chloro-N-[3-(5-cyclopropyl-1-{[(4-methylbenzyl)amino]carbonyl}-1H-pyrazol- |
| | 3-yl)-4-hydroxyphenyl]nicotinamide |
| 2560 | 2-chloro-N-[3-(1-{[(2-chlorobenzyl)amino]carbonyl}-5-cyclopropyl-1H-pyrazol- |
| | 3-yl)-4-hydroxyphenyl]nicotinamide |
| 2561 | 2-chloro-N-[3-(5-cyclopropyl-1-{[(4-fluorobenzyl)amino]carbonyl}-1H-pyrazol- |
| | 3-yl)-4-hydroxyphenyl]nicotinamide |
| 2562 | 2-chloro-N-[3-(5-cyclopropyl-1-{[(3,4-dichlorobenzyl)amino]carbonyl}-1H- |
| | pyrazo1-3-yl)-4-hydroxyphenyl]nicotinamide |
| 2563 | 2-chloro-N-[3-(5-cyclopropyl-1-{[(4-methoxybenzyl)amino]carbonyl}-1H- |
| | pyrazol-3-yl)-4-hydroxyphenyl]nicotinamide |
| 2564 | N-(3-{1-[(sec-butylamino)carbonyl]-5-cyclopropyl-1H-pyrazol-3-yl}-4- |
| | hydroxyphenyl)-2-chloronicotinamide |
| 2565 | 2-chloro-N-[3-(5-cyclopropyl-1-{[(2-fluorobenzyl)amino]carbonyl}-1H-pyrazol- |
| | 3-yl)-4-hydroxyphenyl]nicotinamide |
| 2566 | 2-chloro-N-[3-(5-cyclopropyl-1-{[(3-fluorobenzyl)amino]carbonyl}-1H-pyrazol- |
| | 3-y1)-4-hydroxyphenyl]nicotinamide |
| 2567 | 2-chloro-N-(3-{5-cyclopropyl-1-[(cyclopropylamino)carbonyl]-1H-pyrazol-3-yl}- |
| | 4-hydroxyphenyl)nicotinamide |
| 2568 | 2-chloro-N-(3-{5-cyclobutyl-1-[(isopropylamino)carbonyl]-1H-pyrazol-3-yl}-4- |
| | hydroxyphenyl)nicotinamide |
| 2569 | 2-chloro-N-(3-{5-cyclobutyl-1-[(ethylamino)carbonyl]-1H-pyrazol-3-yl}-4- |
| | hydroxyphenyl)nicotinamide |
| 2570 | N-(3-{1-[(allylamino)carbonyl]-5-cyclobutyl-1H-pyrazol-3-yl}-4-hydroxyphenyl)- |
| | 2-chloronicotinamide |
| 2571 | 2-chloro-N-(3-{5-cyclobutyl-1-[(propylamino)carbonyl]-1H-pyrazol-3-yl}-4- |
| | hydroxyphenyl)nicotinamide |
| 2572 | N-(3-{1-[(benzylamino)carbonyl]-5-cyclobutyl-1H-pyrazol-3-yl}-4- |
| | hydroxyphenyl)-2-chloronicotinamide |
| 2573 | 2-chloro-N-[3-(5-cyclobutyl-1-{[(2-phenylethyl)amino]carbonyl}-1H-pyrazol-3- |
| | yl)-4-hydroxyphenyl]nicotinamide |
| 2574 | 2-chloro-N-[3-(5-cyclobutyl-1-{[(2-methylbenzyl)amino]carbonyl}-1H-pyrazol-3- |
| | yl)-4-hydroxyphenyl]nicotinamide |
| 2575 | 2-chloro-N-[3-(5-cyclobutyl-1-{[(3-methylbenzyl)amino]carbonyl}-1H-pyrazol-3- |
| | yl)-4-hydroxyphenyl]nicotinamide |
| 2576 | 2-chloro-N-[3-(5-cyclobutyl-1-{[(4-methylbenzyl)amino]carbonyl}-1H-pyrazol-3- |
| | yl)-4-hydroxyphenyl]nicotinamide |
| 2577 | 2-chloro-N-[3-(1-{[(2-chlorobenzyl)amino]carbonyl}-5-cyclobutyl-1H-pyrazol-3- |
| | yl)-4-hydroxyphenyl]nicotinamide |
| 2578 | 2-chloro-N-[3-(5-cyclobutyl-1-{[(4-fluorobenzyl)amino]carbonyl}-1H-pyrazol-3- |
| | yl)-4-hydroxyphenyl]nicotinamide |
| 2579 | 2-chloro-N-[3-(5-cyclobutyl-1-{[(3,4-dichlorobenzyl)amino]carbonyl}-1H- |
| | pyrazol-3-yl)-4-hydroxyphenyl]nicotinamide |
| 2580 | 2-chloro-N-[3-(5-cyclobutyl-1-{[(4-methoxybenzyl)amino]carbonyl}-1H-pyrazol- |
| | 3-yl)-4-hydroxyphenyl]nicotinamide |
| 2581 | N-(3-{1-[(sec-butylamino)carbonyl]-5-cyclobutyl-1H-pyrazol-3-yl}-4- |
| | hydroxyphenyl)-2-chloronicotinamide |
| 2582 | 2-chloro-N-[3-(5-cyclobutyl-1-{[(2-fluorobenzyl)amino]carbonyl}-1H-pyrazol-3- |
| | yl)-4-hydroxyphenyl]nicotinamide |
| 2583 | 2-chloro-N-[3-(5-cyclobutyl-1-{[(3-fluorobenzyl)amino]carbonyl}-1H-pyrazol-3- |
| | yl)-4-hydroxyphenyl]nicotinamide |
| 2584 | 2-chloro-N-(3-{5-cyclobutyl-1-[(cyclopropylamino)carbonyl]-1H-pyrazol-3-yl}-4- |
| | hydroxyphenyl)nicotinamide |
| 2585 | 2-chloro-N-(3-{5-cyclopentyl-1-[(isopropylamino)carbonyl]-1H-pyrazol-3-yl}-4- |
| | hydroxyphenyl)nicotinamide |
| 2586 | 2-chloro-N-(3-{5-cyclopentyl-1-[(ethylamino)carbonyl]-1H-pyrazol-3-yl}-4- |
| | hydroxyphenyl)nicotinamide |
| 2587 | N-(3-{1-[(allylamino)carbonyl]-5-cyclopentyl-1H-pyrazol-3-yl}-4- |
| | hydroxyphenyl)-2-chloronicotinamide |
| 2588 | 2-chloro-N-(3-{5-cyclopentyl-1-[(propylamino)carbonyl]-1H-pyrazol-3-yl}-4- |
| | hydroxyphenyl)nicotinamide |
| 2589 | N-(3-{1-[(benzylamino)carbonyl]-5-cyclopentyl-1H-pyrazol-3-yl}-4- |
| | hydroxyphenyl)-2-chloronicotinamide |
| 2590 | 2-chloro-N-[3-(5-cyclopentyl-1-{[(2-phenylethyl)amino]carbonyl}-1H-pyrazol-3- |
| | yl)-4-hydroxyphenyl]nicotinamide |
| 2591 | 2-chloro-N-[3-(5-cyclopentyl-1-{[(2-methylbenzyl)amino]carbonyl}-1H-pyrazol- |
| | 3-yl)-4-hydroxyphenyl]nicotinamide |
| 2592 | 2-chloro-N-[3-(5-cyclopentyl-1-{[(3-methylbenzyl)amino]carbonyl}-1H-pyrazol- |
| | 3-yl)-4-hydroxyphenyl]nicotinamide |
| 2593 | 2-chloro-N-[3-(5-cyclopentyl-1-{[(4-methylbenzyl)amino]carbonyl}-1H-pyrazol- |
| | 3-yl)-4-hydroxyphenyl]nicotinamide |
| 2594 | 2-chloro-N-[3-(1-{[(2-chlorobenzyl)amino]carbonyl}-5-cyclopentyl-1H-pyrazol |
| | 3-yl)-4-hydroxyphenyl]nicotinamide |
| 2595 | 2-chloro-N-[3-(5-cyclopentyl-1-{[(4-flurobenzyl)amino]carbonyl}-1H-pyrazol-3- |
| | yl)-4-hydroxphenyl]nicotinamide |
| 2596 | 2-chloro-N-[3-(5-cyclopentyl-1-{[(3,4-dichlorobenzyl)amino]carbonyl}-1H- |
| | pyrazol-3-yl)-4-hydroxyphenyl]nicotinamide |
| 2597 | 2-chloro-N-[3-(5-cyclopentyl-1-{[(4-methoxybenzyl)amino]carbonyl}-1H- |
| | pyrazol-3-yl)-4-hydroxyphenyl]nicotinamide |
| 2598 | N-(3-{1-[(sec-butylamino)carbonyl]-5-cyclopentyl-1H-pyrazol-3-yl}-4- |
| | hydroxyphenyl)-2-chloronicotinamide |
| 2599 | 2-chloro-N-[3-(5-cyclopentyl-1-{[(2-fluorobenzyl)amino]carbonyl}-1H-pyrazol-3- |
| | yl)-4-hydroxyphenyl]nicotinamide |
| 2600 | 2-chloro-N-[3-(5-cyclopentyl-1-{[(3-fluorobenzyl)amino]carbonyl}-1H-pyrazol-3- |
| | yl)-4-hydroxyphenyl]nicotinamide |
| 2601 | 2-chloro-N-(3-{5-cyclopentyl-1-[(cyclopropylamino)carbonyl]-1H-pyrazol-3-yl}- |
| | 4-hydroxyphenyl)nicotinamide |
| 2602 | 5-cyclopropyl-3-(2-hydroxy-5-{[4-(trifluoromethoxy)benzoyl]amino}phenyl)-N- |
| | isopropyl-1H-pyrazole-1-carboxamide |
| 2603 | 5-cyclopropyl-N-ethyl-3-(2-hydroxy-5-{[4- |
| | (trifluoromethoxy)benzoyl]amino}phenyl)-1H-pyrazole-1-carboxamide |
| 2604 | N-allyl-5-cyclopropyl-3-(2-hydroxy-5-{[4- |
| | (trifluoromethoxy)benzoyl]amino}phenyl)-1H-pyrazole-1-carboxamide |
| 2605 | 5-cyclopropyl-3-(2-hydroxy-5-{[4-(trifluoromethoxy)benzoyl]amino}phenyl)-N- |
| | propyl-1H-pyrazole-1-carboxamide |
| 2606 | N-benzyl-5-cyclopropyl-3-(2-hydroxy-5-{[4- |
| | (trifluoromethoxy)benzoyl]amino}phenyl)-1H-pyrazole-1-carboxamide |
| 2607 | 5-cyclopropyl-3-(2-hydroxy-5-{[4-(trifluoromethoxy)benzoyl]amino}phenyl)-N- |
| | (2-phenylethyl)-1H-pyrazole-1-carboxamide |
| 2608 | 5-cyclopropyl-3-(2-hydroxy-5-{[4-(trifluoromethoxy)benzoyl]amino}phenyl)-N- |
| | (2-methylbenzyl)-1H-pyrazole-1-carboxamide |
| 2609 | 5-cyclopropyl-3-(2-hydroxy-5-{[4-(trifluoromethoxy)benzoyl]amino}phenyl)-N- |
| | (3-methylbenzyl)-1H-pyrazole-1-carboxamide |
| 2610 | 5-cyclopropyl-3-(2-hydroxy-5-{[4-(trifluoromethoxy)benzoyl]amino}phenyl)-N- |
| | (4-methylbenzyl)-1H-pyrazole-1-carboxamide |
| 2611 | N-(2-chlorobenzyl)-5-cyclopropyl-3-(2-hydroxy-5-{[4- |
| | (trifluoromethoxy)benzoyl]amino}phenyl)-1H-pyrazole-1-carboxamide |
| 2612 | 5-cyclopropyl-N-(4-fluorobenzyl)-3-(2-hydroxy-5-{[4- |
| | (trifluoromethoxy)benzoyl]amino}phenyl)-1H-pyrazole-1-carboxamide |
| 2613 | 5-cyclopropyl-N-(3,4-dichlorobenzyl)-3-(2-hydroxy-5-{[4- |
| | (trifluoromethoxy)benzoyl]amino}phenyl)-1H-pyrazole-1-carboxamide |
| 2614 | 5-cyclopropyl-3-(2-hydroxy-5-{[4-(trifluoromethoxy)benzoyl]amino}phenyl)-N- |
| | (4-methoxybenzyl)-1H-pyrazole-1-carboxamide |
| 2615 | N-(sec-butyl)-5-cyclopropyl-3-(2-hydroxy-5-{[4- |
| | (trifluoromethoxy)benzoyl]amino}phenyl)-1H-pyrazole-1-carboxamide |
| 2616 | 5-cyclopropyl-N-(2-fluorobenzyl)-3-(2-hydroxy-5-{[4- |
| | (trifluoromethoxy)benzoyl]amino}phenyl)-1H-pyrazole-1-carboxamide |
| 2617 | 5-cyclopropyl-N-(3-fluorobenzyl)-3-(2-hydroxy-5-{[4- |
| | trifluoromethoxy)benzoyl]amino}phenyl)-1H-pyrazole-1-carboxamide |
| 2618 | N,5-dicyclopropyl-3-(2-hydroxy-5-{[4-(trifluoromethoxy)benzoyl]amino}phenyl)- |
| | 1H-pyrazole-1-carboxamide |
| 2619 | 5-cyclobutyl-3-(2-hydroxy-5-{[4-(trifluoromethoxy)benzoyl]amino}phenyl)-N- |
| | isopropyl-1H-pyrazole-1-carboxamide |
| 2620 | 5-cyclobutyl-N-ethyl-3-(2-hydroxy-5-{[4- |
| | (trifluoromethoxy)benzoyl]amino}phenyl)-1H-pyrazole-1-carboxamide |
| 2621 | N-allyl-5-cyclobutyl-3-(2-hydroxy-5-{[4- |
| | (trifluoromethoxy)benzoyl]amino}phenyl)-1H-pyrazole-1-carboxamide |
| 2622 | 5-cyclobutyl-3-(2-hydroxy-5-{[4-(trifluoromethoxy)benzoyl]amino}phenyl)-N- |
| | propyl-1H-pyrazole-1-carboxamide |
| 2623 | N-benzyl-5-cyclobutyl-3-(2-hydroxy-5-{[4- |
| | (trifluoromethoxy)benzoyl]amino}phenyl)-1H-pyrazole-1-carboxamide |
| 2624 | 5-cyclobutyl-3-(2-hydroxy-5-{[4-(trifluoromethoxy)benzoyl]amino}phenyl)-N-(2- |
| | phenylethyl)-1H-pyrazole-1-carboxamide |
| 2625 | 5-cyclobutyl-3-(2-hydroxy-5-{[4-(trifluoromethoxy)benzoyl]amino}phenyl)-N-(3- |
| | methylbenzyl)-1H-pyrazole-1-carboxamide |
| 2626 | 5-cyclobutyl-3-(2-hydroxy-5-{[4-(trifluoromethoxy)benzoyl]amino}phenyl)-N-(3- |
| | methylbenzyl)-1H-pyrazole-1-carboxamide |
| 2627 | 5-cyclobutyl-3-(2-hydroxy-5-{[4-(trifluoromethoxy)benzoyl]amino}phenyl)-N-(4- |
| | methylbenzyl)-1H-pyrazole-1-carboxamide |
| 2628 | N-(2-chlorobenzyl)-5-cyclobutyl-3-(2-hydroxy-5-{[4- |
| | (trifluoromethoxy)benzoyl]amino}phenyl)-1H-pyrazole-1-carboxamide |
| 2629 | 5-cyclobutyl-N-(4-fluorobenzyl)-3-(2-hydroxy-5-{[4- |
| | (trifluoromethoxy)benzoyl]amino}phenyl)-1H-pyrazole-1-carboxamide |
| 2630 | 5-cyclobutyl-N-(3,4-dichlorobenzyl)-3-(2-hydroxy-5-{[4- |
| | (trifluoromethoxy)benzoyl]amino}phenyl)-1H-pyrazole-1-carboxamide |
| 2631 | 5-cyclobutyl-3-(2-hydroxy-5-{[4-(trifluoromethoxy)benzoyl]amino}phenyl)-N-(4- |
| | methoxybenzyl)-1H-pyrazole-1-carboxamide |
| 2632 | N-(sec-butyl)-5-cyclobutyl-3-(2-hydroxy-5-{[4- |
| | (trifluoromethoxy)benzoyl]amino}phenyl)-1H-pyrazole-1-carboxamide |
| 2633 | 5-cyclobutyl-N-(2-fluorobenzyl)-3-(2-hydroxy-5-{[4- |
| | (trifluoromethoxy)benzoyl]amino}phenyl)-1H-pyrazole-1-carboxamide |
| 2634 | 5-cyclobutyl-N-(3-fluorobenzyl)-3-(2-hydroxy-5-{[4- |
| | (trifluoromethoxy)benzoyl]amino}phenyl)-1H-pyrazole-1-carboxamide |
| 2635 | 5-cyclobutyl-N-cyclopropyl-3-(2-hydroxy-5-{[4- |
| | (trifluoromethoxy)benzoyl]amino}phenyl)-1H-pyrazole-1-carboxamide |
| 2636 | 5-cyclopentyl-3-(2-hydroxy-5-{[4-(trifluoromethoxy)benzoyl]amino}phenyl)-N- |
| | isopropyl-1H-pyrazole-1-carboxamide |
| 2637 | 5-cyclopentyl-N-ethyl-3-(2-hydroxy-5-{[4- |
| | (trifluoromethoxy)benzoyl]amino}phenyl)-1H-pyrazole-1-carboxamide |
| 2638 | N-allyl-5-cyclopentyl-3-(2-hydroxy-5-{[4- |
| | (trifluoromethoxy)benzoyl]amino}phenyl)-1H-pyrazole-1-carboxamide |
| 2639 | 5-cyclopentyl-3-(2-hydroxy-5-{[4-(trifluoromethoxy)benzoyl]amino}phenyl)-N- |
| | propyl-1H-pyrazole-1-carboxamide |
| 2640 | N-benzyl-5-cyclopentyl-3-(2-hydroxy-5-{[4- |
| | (trifluoromethoxy)benzoyl]amino}phenyl)-1H-pyrazole-1-carboxamide |
| 2641 | 5-cyclopentyl-3-(2-hydroxy-5-{[4-(trifluoromethoxy)benzoyl]amino}phenyl)-N- |
| | (2-phenylethyl)-1H-pyrazole-1-carboxamide |
| 2642 | 5-cyclopentyl-3-(2-hydroxy-5-{[4-(trifluoromethoxy)benzoyl]amino}phenyl)-N- |
| | (2-methylbenzyl)-1H-pyrazole-1-carboxamide |
| 2643 | 5-cyclopentyl-3-(2-hydroxy-5-{[4-(trifluoromethoxy)benzoyl]amino}phenyl)-N- |
| | (3-methylbenzyl)-1H-pyrazole-1-carboxamide |
| 2644 | 5-cyclopentyl-3-(2-hydroxy-5-{[4-(trifluoromethoxy)benzoyl]amino}phenyl)-N- |
| | (4-methylbenzyl)-1H-pyrazole-1-carboxamide |
| 2645 | N-(2-chlorobenzyl)-5-cyclopentyl-3-(2-hydroxy-5-{[4- |
| | (trifluoromethoxy)benzoyl]amino}phenyl)-1H-pyrazole-1-carboxamide |
| 2646 | 5-cyclopentyl-N-(4-fluorobenzyl)-3-(2-hydroxy-5-{[4- |
| | (trifluoromethoxy)benzoyl]amino}phenyl)-1H-pyrazole-1-carboxamide |
| 2647 | 5-cyclopentyl-N-(3,4-dichlorobenzyl)-3-(2-hydroxy-5-{[4- |
| | (trifluoromethoxy)benzoyl]amino}phenyl)-1H-pyrazole-1-carboxamide |
| 2648 | 5-cyclopentyl-3-(2-hydroxy-5-{[4-(trifluoromethoxy)benzoyl]amino}phenyl)-N- |
| | (4-methoxybenzyl)-1H-pyrazole-1-carboxamide |
| 2649 | N-(sec-butyl)-5-cyclopentyl-3-(2-hydroxy-5-{[4- |
| | (trifluoromethoxy)benzoyl]amino}phenyl)-1H-pyrazole-1-carboxamide |
| 2650 | 5-cyclopentyl-N-(2-fluorobenzyl)-3-(2-hydroxy-5-{[4- |
| | (trifluoromethoxy)benzoyl]amino}phenyl)-1H-pyrazole-1-carboxamide |
| 2651 | 5-cyclopentyl-N-(3-fluorobenzyl)-3-(2-hydroxy-5-{[4- |
| | (trifluoromethoxy)benzoyl]amino}phenyl)-1H-pyrazole-1-carboxamide |
| 2652 | 5-cyclopentyl-N-cyclopropyl-3-(2-hydroxy-5-{[4- |
| | (trifluoromethoxy)benzoyl]amino}phenyl)-1H-pyrazole-1-carboxamide |
| 2653 | N-(3-{5-cyclopropyl-1-[(isopropylamino)carbonyl]-1H-pyrazol-3-yl}-4- |
| | hydroxyphenyl)isoxazole-5-carboxamide |
| 2654 | N-(3-{5-cyclopropyl-1-[(ethylamino)carbonyl]-1H-pyrazol-3-yl}-4- |
| | hydroxyphenyl)isoxazole-5-carboxamide |
| 2655 | N-(3-{1-[(allylamino)carbonyl]-5-cyclopropyl-1H-pyrazol-3-yl}-4- |
| | hydroxyphenyl)isoxazole-5-carboxamide |
| 2656 | N-(3-{5-cyclopropyl-1-[(propylamino)carbonyl]-1H-pyrazol-3-yl}-4- |
| | hydroxyphenyl)isoxazole-5-carboxamide |
| 2657 | N-(3-{1-[(benzylamino)carbonyl]-5-cyclopropyl-1H-pyrazol-3-yl}-4- |
| | hydroxyphenyl)isoxazole-5-carboxamide |
| 2658 | N-[3-(5-cyclopropyl-1-{[(2-phenylethyl)amino]carbonyl}-1H-pyrazol-3-yl)-4- |
| | hydroxyphenyl]isoxazole-5-carboxamide |
| 2659 | N-[3-(5-cyclopropyl-1-{[(2-methylbenzyl)amino]carbonyl}-1H-pyrazol-3-yl)-4- |
| | hydroxyphenyl]isoxazole-5-carboxamide |
| 2660 | N-[3-(5-cyclopropyl-1-{[(3-methylbenzyl)amino]carbonyl}-1H-pyrazol-3-yl)-4- |
| | hydroxyphenyl]isoxazole-5-carboxamide |
| 2661 | N-[3-(5-cyclopropyl-1-{[(4-methylbenzyl)amino]carbonyl}-1H-pyrazol-3-yl)-4- |
| | hydroxyphenyl]isoxazole-5-carboxamide |
| 2662 | N-[3-(1-{[(2-chlorobenzyl)amino]carbonyl}-5-cyclopropyl-1H-pyrazol-3-yl)-4- |
| | hydroxyphenyl]isoxazole-5-carboxamide |
| 2663 | N-[3-(5-cyclopropyl-1-{[(4-fluorobenzyl)amino]carbonyl}-1H-pyrazol-3-yl)-4- |
| | hydroxyphenyl]isoxazole-5-carboxamide |
| 2664 | N-[3-(5-cyclopropyl-1-{[(3,4-dichlorobenzyl)amino]carbonyl}-1H-pyrazol-3-yl)- |
| | 4-hydroxyphenyl]isoxazole-5-carboxamide |
| 2665 | N-[3-(5-cyclopropyl-1-{[(4-methoxybenzyl)amino]carbonyl}-1H-pyrazol-3-yl)-4- |
| | hydroxyphenyl]isoxazole-5-carboxamide |
| 2666 | N-(3-{1-[(sec-butylamino)carbonyl]-5-cyclopropyl-1H-pyrazol-3-yl}-4- |
| | hydroxyphenyl)isoxazole-5-carboxamide |
| 2667 | N-[3-(5-cyclopropyl-1-{[(2-fluorobenzyl)amino]carbonyl}-1H-pyrazol-3-yl)-4- |
| | hydroxyphenyl]isoxazole-5-carboxamide |
| 2668 | N-[3-(5-cyclopropyl-1-{[(3-fluorobenzyl)amino]carbonyl}-1H-pyrazol-3-yl)-4- |
| | hydroxyphenyl]isoxazole-5-carboxamide |
| 2669 | N-(3-{5-cyclopropyl-1-[(cyclopropylamino)carbonyl]-1H-pyrazol-3-yl}-4- |
| | hydroxyphenyl)isoxazole-5-carboxamide |
| 2670 | N-(3-{5-cyclobutyl-1-[(isopropylamino)carbonyl]-1H-pyrazol-3-yl}-4- |
| | hydroxyphenyl)isoxazole-5-carboxamide |
| 2671 | N-(3-{5-cyclobutyl-1-[(ethylamino)carbonyl]-1H-pyrazol-3-yl}-4- |
| | hydroxyphenyl)isoxazole-5-carboxamide |
| 2672 | N-(3-{1-[(allylamino)carbonyl]-5-cyclobutyl-1H-pyrazol-3-yl}-4- |
| | hydroxyphenyl)isoxazole-5-carboxamide |
| 2673 | N-(3-{5-cyclobutyl-1-[(propylamino)carbonyl]-1H-pyrazol-3-yl}-4- |
| | hydroxyphenyl)isoxazole-5-carboxamide |
| 2674 | N-(3-{1-[(benzylamino)carbonyl]-5-cyclobutyl-1H-pyrazol-3-yl}-4- |
| | hydroxyphenyl)isoxazole-5-carboxamide |
| 2675 | N-[3-(5-cyclobutyl-1-{[(2-phenylethyl)amino]carbonyl}-1H-pyrazol-3-yl)-4- |
| | hydroxyphenyl]isoxazole-5-carboxamide |
| 2676 | N-[3-(5-cyclobutyl-1-{[(2-methylbenzyl)amino]carbonyl}-1H-pyrazol-3-yl)-4- |
| | hydroxyphenyl]isoxazole-5-carboxamide |
| 2677 | N-[3-(5-cyclobutyl-1-{[(3-methylbenzyl)amino]carbonyl}-1H-pyrazol-3-yl)-4- |
| | hydroxyphenyl]isoxazole-5-carboxamide |
| 2678 | N-[3-(5-cyclobutyl-1-{[(4-methylbenzyl)amino]carbonyl}-1H-pyrazol-3-yl)-4- |
| | hydroxyphenyl]isoxazole-5-carboxamide |
| 2679 | N-[3-(1-{[(2-chlorobenzyl)amino]carbonyl}-5-cyclobutyl-1H-pyrazol-3-yl)-4- |
| | hydroxyphenyl]isoxazole-5-carboxamide |
| 2680 | N-[3-(5-cyclobutyl-1-{[(4-fluorobenzyl)amino]carbonyl}-1H-pyrazol-3-yl)-4- |
| | hydroxyphenyl]isoxazole-5-carboxamide |
| 2681 | N-[3-(5-cyclobutyl-1-{[(3,4-dichlorobenzyl)amino]carbonyl}-1H-pyrazol-3-yl)-4- |
| | hydroxyphenyl]isoxazole-5-carboxamide |
| 2682 | N-[3-(5-cyclobutyl-1-{[(4-methoxybenzyl)amino]carbonyl}-1H-pyrazol-3-yl)-4- |
| | hydroxyphenyl]isoxazo1e-5-carboxamide |
| 2683 | N-(3-{1-[(sec-butylamino)carbonyl]-5-cyclobutyl-1H-pyrazol-3-yl}-4- |
| | hydroxyphenyl)isoxazole-5-carboxamide |
| 2684 | N-[3-(5-cyclobutyl-1-{[(2-fluorobenzyl)amino]carbonyl}-1H-pyrazol-3-yl)-4- |
| | hydroxyphenyl]isoxazole-5-carboxamide |
| 2685 | N-[3-(5-cyclobutyl-1-{[(3-fluorobenzyl)amino]carbonyl}-1H-pyrazol-3-yl)-4- |
| | hydroxyphenyl]isoxazole-5-carboxamide |
| 2686 | N-(3-{5-cyclobutyl-1-[(cyclopropylamino)carbonyl]-1H-pyrazol-3-yl}-4- |
| | hydroxyphenyl)isoxazole-5-carboxamide |
| 2687 | N-(3-{5-cyclopentyl-1-[(isopropylamino)carbonyl]-1H-pyrazol-3-yl}-4- |
| | hydroxyphenyl)isoxazole-5-carboxamide |
| 2688 | N-(3-{5-cyclopentyl-1-[(ethylamino)carbonyl]-1H-pyrazol-3-yl}-4- |
| | hydroxyphenyl)isoxazole-5-carboxamide |
| 2689 | N-(3-{1-[(allylamino)carbonyl]-5-cyclopentyl-1H-pyrazol-3-yl}-4- |
| | hydroxyphenyl)isoxazole-5-carboxamide |
| 2690 | N-(3-{5-cyclopentyl-1-[(propylamino)carbonyl]-1H-pyrazol-3-yl}-4- |
| | hydroxyphenyl)isoxazole-5-carboxamide |
| 2691 | N-(3-{1-[(benzylamino)carbonyl]-5-cyclopentyl-1H-pyrazol-3-yl}-4- |
| | hydroxyphenyl)isoxazole-5-carboxamide |
| 2692 | N-[3-(5-cyclopentyl-1-{[(2-phenylethyl)amino]carbonyl}-1H-pyrazol-3-yl)-4- |
| | hydroxyphenyl]isoxazole-5-carboxamide |
| 2693 | N-[3-(5-cyclopentyl-1-{[(2-methylbenzyl)amino]carbonyl}-1H-pyrazol-3-yl)-4- |
| | hydroxyphenyl]isoxazole-5-carboxamide |
| 2694 | N-[3-(5-cyclopentyl-1-{[(3-methylbenzyl)amino]carbonyl}-1H-pyrazol-3-yl)-4- |
| | hydroxyphenyl]isoxazole-5-carboxamide |
| 2695 | N-[3-(5-cyclopentyl-1-{[(4-methylbenzyl)amino]carbonyl}-1H-pyrazol-3-yl)-4- |
| | hydroxyphenyl]isoxazole-5-carboxamide |
| 2696 | N-[3-(1-{[(2-chlorobenzyl)amino]carbonyl}-5-cyclopentyl-1H-pyrazol-3-yl)-4- |
| | hydroxyphenyl]isoxazole-5-carboxamide |
| 2697 | N-[3-(5-cyclopentyl-1-{[(4-fluorobenzyl)amino]carbonyl}-1H-pyrazol-3-yl)-4- |
| | hydroxyphenyl]isoxazole-5-carboxamide |
| 2698 | N-[3-(5-cyclopentyl-1-{[(3,4-dichlorobenzyl)amino]carbonyl}-1H-pyrazol-3-yl)- |
| | 4-hydroxyphenyl]isoxazole-5-carboxamide |
| 2699 | N-[3-(5-cyclopentyl-1-{[(4-methoxybenzyl)amino]carbonyl}-1H-pyrazol-3-yl)-4- |
| | hydroxyphenyl]isoxazole-5-carboxamide |
| 2700 | N-(3-{1-[(sec-butylamino)carbonyl]-5-cyclopentyl-1H-pyrazol-3-yl}-4- |
| | hydroxyphenyl)isoxazole-5-carboxamide |
| 2701 | N-[3-(5-cyclopentyl-1-{[(2-fluorobenzyl)amino]carbonyl}-1H-pyrazol-3-yl)-4- |
| | hydroxyphenyl]isoxazole-5-carboxamide |
| 2702 | N-[3-(5-cyclopentyl-1-{[(3-fluorobenzyl)amino]carbonyl}-1H-pyrazol-3-yl)-4- |
| | hydroxyphenyl]isoxazole-5-carboxamide |
| 2703 | N-(3-{5-cyclopentyl-1-[(cyclopropylamino)carbonyl]-1H-pyrazol-3-yl}-4- |
| | hydroxyphenyl)isoxazole-5-carboxamide |
| 2704 | 5-cyclopropyl-3-(2-hydroxy-5-{[(1-methyl-1H-pyrrol-2- |
| | yl)carbonyl]amino}phenyl)-N-isopropyl-1H-pyrazole-1-carboxamide |
| 2705 | 5-cyclopropyl-N-ethyl-3-(2-hydroxy-5-{[(1-methyl-1H-pyrrol-2- |
| | yl)carbonyl]amino}phenyl)-1H-pyrazole-1-carboxamide |
| 2706 | N-allyl-5-cyclopropyl-3-(2-hydroxy-5-{[(1-methyl-1H-pyrrol-2- |
| | yl)carbonyl]amino}phenyl)-1H-pyrazole-1-carboxamide |
| 2707 | 5-cyclopropyl-3-(2-hydroxy-5-{[(1-methyl-1H-pyrrol-2- |
| | yl)carbonyl]amino}phenyl)-N-propyl-1H-pyrazole-1-carboxamide |
| 2708 | N-benzyl-5-cyclopropyl-3-(2-hydroxy-5-{[(1-methyl-1H-pyrrol-2- |
| | yl)carbonyl]amino}phenyl)-1H-pyrazole-1-carboxamide |
| 2709 | 5-cyclopropyl-3-(2-hydroxy-5-{[(1-methyl-1H-pyrrol-2- |
| | yl)carbonyl]amino}phenyl)-N-(2-phenylethyl)-1H-pyrazole-1-carboxamide |
| 2710 | 5-cyclopropyl-3-(2-hydroxy-5-{[(1-methyl-1H-pyrrol-2- |
| | yl)carbonyl]amino}phenyl)-N-(2-methylbenzyl)-1H-pyrazole-1-carboxamide |
| 2711 | 5-cyclopropyl-3-(2-hydroxy-5-{[(1-methyl-1H-pyrrol-2- |
| | yl)carbonyl]amino}phenyl)-N-(3-methylbenzyl)-1H-pyrazole-1-carboxamide |
| 2712 | 5-cyclopropyl-3-(2-hydroxy-5-{[(1-methyl-1H-pyrrol-2- |
| | yl)carbonyl]amino}phenyl)-N-(4-methylbenzyl)-1H-pyrazole-1-carboxamide |
| 2713 | N-(2-chlorobenzyl)-5-cyclopropyl-3-(2-hydroxy-5-{[(1-methyl-1H-pyrrol-2- |
| | yl)carbonyl]amino}phenyl)-1H-pyrazole-1-carboxamide |
| 2714 | 5-cyclopropyl-N-(4-fluorobenzyl)-3-(2-hydroxy-5-{[(1-methyl-1H-pyrrol-2- |
| | yl)carbonyl]amino}phenyl)-1H-pyrazole-1-carboxamide |
| 2715 | 5-cyclopropyl-N-(3,4-dichlorobenzyl)-3-(2-hydroxy-5-{[(1-methyl-1H-pyrrol-2- |
| | yl)carbonyl]amino}phenyl)-1H-pyrazole-1-carboxamide |
| 2716 | 5-cyclopropyl-3-(2-hydroxy-5-{[(1-methyl-1H-pyrrol-2- |
| | yl)carbonyl]amino}phenyl)-N-(4-methoxybenzyl)-1H-pyrazole-1-carboxamide |
| 2717 | N-(sec-butyl)-5-cyclopropyl-3-(2-hydroxy-5-{[(1-methyl-1H-pyrrol-2- |
| | yl)carbonyl]amino}phenyl)-1H-pyrazole-1-carboxamide |
| 2718 | 5-cyclopropyl-N-(2-fluorobenzyl)-3-(2-hydroxy-5-{[(1-methyl-1H-pyrrol-2- |
| | yl)carbonyl]amino}phenyl)-1H-pyrazole-1-carboxamide |
| 2719 | 5-cyclopropyl-N-(3-fluorobenzyl)-3-(2-hydroxy-5-{[(1-methyl-1H-pyrrol-2- |
| | yl)carbonyl]amino}phenyl)-1H-pyrazole-1-carboxamide |
| 2720 | N,5-dicyclopropyl-3-(2-hydroxy-5-{[(1-methyl-1H-pyrrol-2- |
| | yl)carbonyl]amino}phenyl)-1H-pyrazole-1-carboxamide |
| 2721 | 5-cyclobutyl-3-(2-hydroxy-5-{[(1-methyl-1H-pyrrol-2- |
| | yl)carbonyl]amino}phenyl)-N-isopropyl-1H-pyrazole-1-carboxamide |
| 2722 | 5-cyclobutyl-N-ethyl-3-(2-hydroxy-5-{[(1-methyl-1H-pyrrol-2- |
| | yl)carbonyl]amino}phenyl)-1H-pyrazole-1-carboxamide |
| 2723 | N-allyl-5-cyclobutyl-3-(2-hydroxy-5-{[(1-methyl-1H-pyrrol-2- |
| | yl)carbonyl]amino}phenyl)-1H-pyrazole-1-carboxamide |
| 2724 | 5-cyclobutyl-3-(2-hydroxy-5-{[(1-methyl-1H-pyrrol-2- |
| | yl)carbonyl]amino}phenyl)-N-propyl-1H-pyrazole-1-carboxamide |
| 2725 | N-benzyl-5-cyclobutyl-3-(2-hydroxy-5-{[(1-methyl-1H-pyrrol-2- |
| | yl)carbonyl]amino}phenyl)-1H-pyrazole-1-carboxamide |
| 2726 | 5-cyclobutyl-3-(2-hydroxy-5-{[(1-methyl-1H-pyrrol-2- |
| | yl)carbonyl]amino}phenyl)-N-(2-phenylethyl)-1H-pyrazole-1-carboxamide |
| 2727 | 5-cyclobutyl-3-(2-hydroxy-5-{[(1-methyl-1H-pyrrol-2- |
| | yl)carbonyl]amino}phenyl)-N-(2-methylbenzyl)-1H-pyrazole-1-carboxamide |
| 2728 | 5-cyclobutyl-3-(2-hydroxy-5-{[(1-methyl-1H-pyrrol-2- |
| | yl)carbonyl]amino}phenyl)-N-(3-methylbenzyl)-1H-pyrazole-1-carboxamide |
| 2729 | 5-cyclobutyl-3-(2-hydroxy-5-{[(1-methyl-1H-pyrrol-2- |
| | yl)carbonyl]amino}phenyl)-N-(4-methylbenzyl)-1H-pyrazole-1-carboxamide |
| 2730 | N-(2-chlorobenzyl)-5-cyclobutyl-3-(2-hydroxy-5-{[(1-methyl-1H-pyrrol-2- |
| | yl)carbonyl]amino}phenyl)-1H-pyrazole-1-carboxamide |
| 2731 | 5-cyclobutyl-N-(4-fluorobenzyl)-3-(2-hydroxy-5-{[(1-methyl-1H-pyrrol-2- |
| | yl)carbonyl]amino}phenyl)-1H-pyrazole-1-carboxamide |
| 2732 | 5-cyclobutyl-N-(3,4-dichlorobenzyl)-3-(2-hydroxy-5-{[(1-methyl-1H-pyrrol-2- |
| | yl)carbonyl]amino}phenyl)-1H-pyrazole-1-carboxamide |
| 2733 | 5-cyclobutyl-3-(2-hydroxy-5-{[(1-methyl-1H-pyrrol-2- |
| | yl)carbonyl]amino}phenyl)-N-(4-methoxybenzyl)-1H-pyrazole-1-carboxamide |
| 2734 | N-(sec-butyl)-5-cyclobutyl-3-(2-hydroxy-5-{[(1-methyl-1H-pyrrol-2- |
| | yl)carbonyl]amino}phenyl)-1H-pyrazole-1-carboxamide |
| 2735 | 5-cyclobutyl-N-(2-fluorobenzyl)-3-(2-hydroxy-5-{[(1-methyl-1H-pyrrol-2- |
| | yl)carbonyl]amino}phenyl)-1H-pyrazole-1-carboxamide |
| 2736 | 5-cyclobutyl-N-(3-fluorobenzyl)-3-(2-hydroxy-5-{[(1-methyl-1H-pyrrol-2- |
| | yl)carbonyl]amino}phenyl)-1H-pyrazole-1-carboxamide |
| 2737 | 5-cyclobutyl-N-cyclopropyl-3-(2-hydroxy-5-{[(1-methyl-1H-pyrrol-2- |
| | yl)carbonyl]amino}phenyl)-1H-pyrazole-1-carboxamide |
| 2738 | 5-cyclopentyl-3-(2-hydroxy-5-{[(1-methyl-1H-pyrrol-2- |
| | yl)carbonyl]amino}phenyl)-N-isopropyl-1H-pyrazole-1-carboxamide |
| 2739 | 5-cyclopentyl-N-ethyl-3-(2-hydroxy-5-{[(1-methyl-1H-pyrrol-2- |
| | yl)carbonyl]amino}phenyl)-1H-pyrazole-1-carboxamide |
| 2740 | N-allyl-5-cyclopentyl-3-(2-hydroxy-5-{[(1-methyl-1H-pyrrol-2- |
| | yl)carbonyl]amino}phenyl)-1H-pyrazole-1-carboxamide |
| 2741 | 5-cyclopentyl-3-(2-hydroxy-5-{[(1-methyl-1H-pyrrol-2- |
| | yl)carbonyl]amino}phenyl)-N-propyl-1H-pyrazole-1-carboxamide |
| 2742 | N-benzyl-5-cyclopentyl-3-(2-hydroxy-5-{[(1-methyl-1H-pyrrol-2- |
| | yl)carbonyl]amino}phenyl)-1H-pyrazole-1-carboxamide |
| 2743 | 5-cyclopentyl-3-(2-hydroxy-5-{[(1-methyl-1H-pyrrol-2- |
| | yl)carbonyl]amino}phenyl)-N-(2-phenylethyl)-1H-pyrazole-1-carboxamide |
| 2744 | 5-cyclopentyl-3-(2-hydroxy-5-{[(1-methyl-1H-pyrrol-2- |
| | yl)carbonyl]amino}phenyl)-N-(2-methylbenzyl)-1H-pyrazole-1-carboxamide |
| 2745 | 5-cyclopentyl-3-(2-hydroxy-5-{[(1-methyl-1H-pyrrol-2- |
| | yl)carbonyl]amino}phenyl)-N-(3-methylbenzyl)-1H-pyrazole-1-carboxamide |
| 2746 | 5-cyclopentyl-3-(2-hydroxy-5-{[(1-methyl-1H-pyrrol-2- |
| | yl)carbonyl]amino}phenyl)-N-(4-methylbenzyl)-1H-pyrazole-1-carboxamide |
| 2747 | N-(2-chlorobenzyl)-5-cyclopentyl-3-(2-hydroxy-5-{[(1-methyl-1H-pyrrol-2- |
| | yl)carbonyl]amino}phenyl)-1H-pyrazole-1-carboxamide |
| 2748 | 5-cyclopentyl-N-(4-fluorobenzyl)-3-(2-hydroxy-5-{[(1-methyl-1H-pyrrol-2- |
| | yl)carbonyl]amino}phenyl)-1H-pyrazole-1-carboxamide |
| 2749 | 5-cyclopentyl-N-(3,4-dichlorobenzyl)-3-(2-hydroxy-5-{[(1-methyl-1H-pyrrol-2- |
| | yl)carbonyl]amino}phenyl)-1H-pyrazole-1-carboxamide |
| 2750 | 5-cyclopentyl-3-(2-hydroxy-5-{[(1-methyl-1H-pyrrol-2- |
| | yl)carbonyl]amino}phenyl)-N-(4-methoxybenzyl)-1H-pyrazole-1-carboxamide |
| 2751 | N-(sec-butyl)-5-cyclopentyl-3-(2-hydroxy-5-{[(1-methyl-1H-pyrrol-2- |
| | yl)carbonyl]amino}phenyl)-1H-pyrazole-1-carboxamide |
| 2752 | 5-cyclopentyl-N-(2-fluorobenzyl)-3-(2-hydroxy-5-{[(1-methyl-1H-pyrrol-2- |
| | yl)carbonyl]amino}phenyl)-1H-pyrazole-1-carboxamide |
| 2753 | 5-cyclopentyl-N-(3-fluorobenzyl)-3-(2-hydroxy-5-{[(1-methyl-1H-pyrrol-2- |
| | yl)carbonyl]amino}phenyl)-1H-pyrazole-1-carboxamide |
| 2754 | 5-cyclopentyl-N-cyclopropyl-3-(2-hydroxy-5-{[(1-methyl-1H-pyrrol-2- |
| | yl)carbonyl]amino}phenyl)-1H-pyrazole-1-carboxamide |

1. A method for treating diseases caused by and/or associated with an altered protein kinase activity which comprises administering to a mammal in need thereof an effective amount of a hydroxyphenyl-pyrazole derivative represented by formula (I): 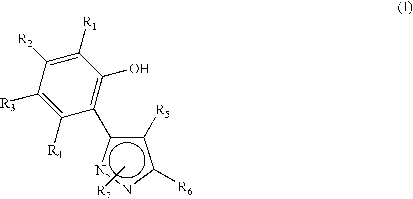
wherein R₁ to R₄ independently represent
hydrogen or halogen atom, hydroxy, nitro or NR₈R₉ group, wherein R₈ and R₉ independently represent hydrogen or an optionally substituted group selected from C₁-C₆ alkyl, aryl, aryl C₁-C₆ alkyl, C₃-C₇ cycloalkyl and saturated or unsaturated heterocyclyl group, or a residue of formula COR₁₀, CONHR₁₀ or SO₂R₁₀ in which R₁₀ is hydrogen atom or an optionally substituted group selected from C₁-C₆ alkyl, aryl, aryl C₁-C₆ alkyl, saturated or unsaturated C₃-C₇ cycloalkyl or saturated or unsaturated heterocyclyl group, or
an optionally substituted group selected from a straight or branched C₁-C₆ alkyl, aryl, aryl C₁-C₆ alkyl, a saturated or unsaturated C₃-C₇ cycloalkyl or cycloalkoxy group, saturated or unsaturated heterocyclyl, C₁-C₆ alkoxy, aryloxy, aryl C₁-C₆ alkoxy;
R₅ and R₆ independently represent hydrogen or an optionally substituted group selected from C₁-C₆ alkyl, aryl, aryl C₁-C₆ alkyl, C₃-C₇ cycloalkyl and saturated or unsaturated heterocyclyl,
R₇ is a substituent attached at one of the two nitrogen atoms of the pyrazole ring having the formula CONHR₁₀, CSNHR₁₀, SO₂R₁₀, COR₁₀ or COOR₁₀, in which R₁₀ is as above defined, or a pharmaceutically acceptable salt thereof.

2. The method of claim 1 wherein the disease caused by and/or associated with an altered protein kinase activity is selected from the group consisting of cancer, cell proliferative disorders, Alzheimer's disease, viral infections, auto-immune diseases and neurodegenerative disorders.

3. The method of claim 2 wherein the cancer is selected from carcinoma, squamous cell carcinoma, hematopoietic tumors of myeloid or lymphoid lineage, tumors of mesenchymal origin, tumors of the central and peripheral nervous system, melanoma, seminoma, teratocarcinoma, osteosarcoma, xeroderma pigmentosum, keratocanthoma, thyroid follicular cancer and Kaposi's sarcoma.

4. The method of claim 2 wherein the cell proliferative disorder is selected from the group consisting of benign prostate hyperplasia, familial adenomatosis polyposis, neuro-fibromatosis, psoriasis, vascular smooth cell proliferation associated with atherosclerosis, pulmonary fibrosis, arthritis glomerulonephritis and post-surgical stenosis and restenosis.

5. The method of claim 1 which provides tumor angiogenesis and metastasis inhibition.

6. The method of claim 1 further comprising subjecting the mammal in need thereof to a radiation therapy or chemotherapy regimen in combination with at least one cytostatic or cytotoxic agent.

7. The method of claim 1 wherein the mammal in need thereof is a human.

8. The method of claim 1 wherein in the compound of formula (I), the R₇ substituent is attached at the nitrogen atom at 1 position and has the following formula (I′) 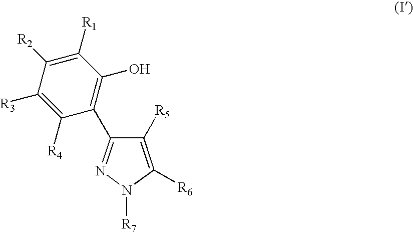
wherein R₁ to R₄, R₅, R₆ and R₇ are as defined in claim 1, or a pharmaceutically acceptable salt thereof.

9. The method of claim 1 wherein, within formula (I), R₇ is —CONHR₁₀ group wherein R₁₀ is as defined in claim 1.

10. The method of claim 1 wherein, within formula (I), one of R₂ and R₃ is a NHCOR₁₀ or NHCSR₁₀ group, wherein R₁₀ is as defined in claim 1.

11. A method for inhibiting protein kinase activity which comprises contacting the said kinase with an effective amount of a compound as defined in claim 1.

12. A hydroxyaryl-pyrazole derivative represented by formula (I): 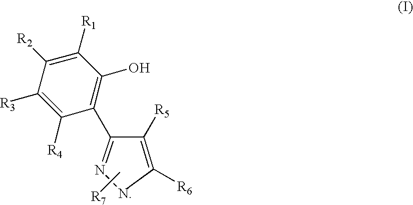
wherein R1 to R independently represent
hydrogen or halogen atom, hydroxy, nitro or NR₈R₉ group, wherein R₈ and R₉ independently represent hydrogen or an optionally substituted group selected from C₁-C₆ alkyl, aryl, aryl C₁-C₆ alkyl, C₃-C₇ cycloalkyl and saturated or unsaturated heterocyclyl group, or a residue of formula COR₁₀, CONHR₁₀ or SO₂R₁₀ in which R₁₀ is hydrogen atom or an optionally substituted group selected from C₁-C₆ alkyl, aryl, aryl C₁-C₆ alkyl, saturated or unsaturated C₃-C₇ cycloalkyl or saturated or unsaturated heterocyclyl group, or
an optionally substituted group selected from a straight or branched C₁-C₆ alkyl, aryl, aryl C₁-C₆ alkyl, a saturated or unsaturated C₃-C₆ cycloalkyl or cycloalkoxy group, saturated or unsaturated heterocyclyl, C₁-C₆ alkoxy, aryloxy, aryl C₁-C₆ alkoxy;
R₅ and R₆ independently represent hydrogen or an optionally substituted group selected from C₁-C₆ alkyl, aryl, aryl C₁-C₆ alkyl, C₃-C₇ cycloalkyl and saturated or unsaturated heterocyclyl,
R₇ is a substituent attached at one of the two nitrogen atoms of the pyrazole ring having the formula CONHR₁₀, CSNHR₁₀, SO₂R₁₀, COR₁₀ or COOR₁,o in which R₁₀ is as above defined, with the following provisos:
when R₇ is CONH₂ or CSNH₂ and R₅ is H or CH₃, then R₆ is not H, CH₃ or phenyl group,
when R₇ is CO R′₁₁ in which R′₁₁ is an optionally substituted C₁-C₆ alkyl, phenyl, or saturated or unsaturated heterocyclyl group, and R₅ is H or phenyl group, then R₆ is not H, CH₃ or an optionally substituted phenyl,
when R₇ is CO₂C(CH₃)₃ or CO₂CH₃ and R₅ is H, then R₆ is not an optionally substituted phenyl group;
or a pharmaceutically acceptable salt thereof.

13. A hydroxyaryl-pyrazole derivative according to claim 12 of formula (I′) 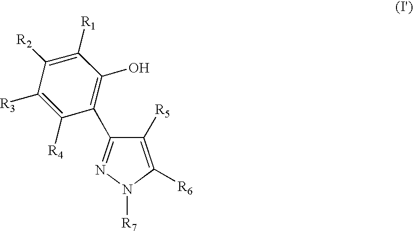
wherein wherein R₁, R₄ and R₅ are hydrogen atoms, R₂ and R₃ are independently selected from
hydrogen or halogen atom,
an optionally substituted straight or branched C₁-C₆ alkyl or C₁-C₆ alkoxy, and —NR₈R₉ group, wherein R₈ and R₉ independently represent hydrogen atom or a residue of formula COR₁₀ wherein R₁₀ is an optionally substituted group selected from aryl and saturated or unsaturated heterocyclyl group.

14. A compound of formula (I′) according to claim 13, wherein R₂ and R₃ are selected from hydrogen, chlorine or fluorine atom, optionally substituted straight or branched C₁-C₆ alkyl or C₁-C₆ alkoxy and NR₈R₉ group, wherein R₈ and R₉ independently represent hydrogen atom or a residue of formula COR₁₀ wherein R₁₀ is an optionally substituted group selected from phenyl, 1-naphthyl, 2-naphtyl, biphenyl, pyridyl, pyrazolyl, thienyl, isoxazolyl, thiazolyl, pyrazolyl, fluorene-9-yl, piperidine or tetrahydroquinoline.

15. A hydroxyaryl-pyrazole derivative according to claim 12 of formula (I′): 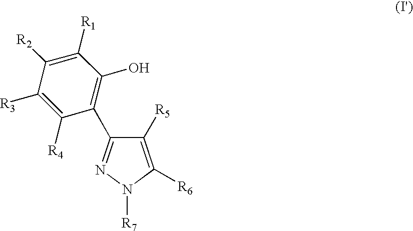
wherein R₆ is an optionally substituted group selected from C₁-C₆ alkyl, aryl, C₃-C₇ cycloalkyl and saturated or unsaturated heterocyclyl group.

16. A compound of formula (I′) according to claim 15, wherein R₆ is an optionally substituted group selected from straight or branched C₁-C₆ alkyl, C₃-C₇ cycloalkyl, phenyl, 1-naphthyl, 2-naphtyl, biphenyl, pyridyl, pyrazolyl, thienyl, isoxazolyl, thiazolyl, pyrazolyl, fluorene-9-yl, piperidine or tetrahydroquinoline.

17. A hydroxyaryl-pyrazole derivative according to claim 12 of formula (I′): 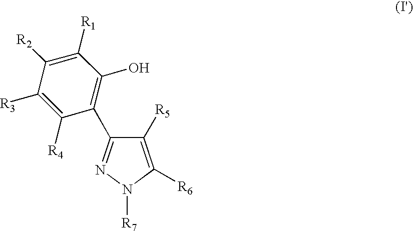
wherein R₇ represents a group of formula CONHR₁₀, in which R₁₀ is hydrogen atom or an optionally substituted group selected from C₁-C₆ alkyl, aryl, aryl C₁-C₆ alkyl, C₂-C₈ alkenyl, C₃-C₇ cycloalkyl or saturated or unsaturated heterocyclyl group.

18. A compound of formula (I′) according to claim 17, wherein R₇ represents a group of formula CONHR₁₀, in which R₁₀ is C₁-C₆ alkyl, phenyl, 1-naphthyl, 2-naphtyl, biphenyl, benzyl, allyl or phenethyl group.

19. A compound of formula (I) according to claim 12 which is selected from:
Ia. 5-Cyclopropyl-3-[2-hydroxy-4-(4-methoxy-benzoylamino)-phenyl]-pyrazole-1-carboxylic acid butylamide;
Ib. N-ethyl-3-(2-hydroxyphenyl)-5-pyridin-4-yl-1H-pyrazole-1-carboxamide;
Ic. 5-cyclopropyl-3-(2-hydroxyphenyl)-N-propyl-1H-pyrazole-1-carboxamide;
Id. ethyl N-{[5-cyclopropyl-3-(2-hydroxyphenyl)-1H-pyrazol-1-yl]carbonyl}glycinate;
Ie. N-allyl-5-cyclopropyl-3-(2-hydroxyphenyl)-1H-pyrazole-1-carboxamide;
If. N-cyclohexyl-5-cyclopropyl-3-(2-hydroxyphenyl)-1H-pyrazole-1-carboxamide;
Ig. 5-cyclopropyl-N-ethyl-3-(2-hydroxy-4-{[3-(trifluoromethyl)benzoyl]amino}phenyl)-1H-pyrazole-1-carboxamide;
Ih. ethyl-N-{[5-[2-(dimethylamino)ethyl]-3-(2-hydroxyphenyl)-1H-pyrazol-1-yl]carbonyl}glycinate;
Ii. 5-cyclopropyl-N-ethyl-3-(2-hydroxy-4-methoxyphenyl)-1H-pyrazole-1-carboxamide;
Ij. ethyl-N-{[5-cyclopropyl-3-(2-hydroxy-4-methoxyphenyl)-1H-pyrazol-1-yl]carbonyl}glycinate;
Ik. 5-cyclopropyl-N-ethyl-3-(2-hydroxy-5-methoxyphenyl)-1H-pyrazole-1-carboxamide;
Il. 5-cyclopropyl-N-ethyl-3-(2-hydroxy-5-methylphenyl)-1H-pyrazole-1-carboxamide;
Im. ethyl-N-{[5-cyclopropyl-3-(2-hydroxy-5-methylphenyl)-1H-pyrazol-1-yl]carbonyl}glycinate;
In. N-butyl-5-cyclopropyl-3-(2-hydroxy-4-methoxyphenyl)-1H-pyrazole-1-carboxamide:
Io. N-butyl-5-cyclopropyl-3-(2-hydroxy-5-methylphenyl)-1H-pyrazole-1-carboxamide;
Ip. N-(3-{1-[(butylamino)carbonyl]-5-cyclopropyl-1H-pyrazol-3-yl}-4-hydroxyphenyl)isonicotinamide;
Iq. N-(4-{1-[(butylamino)carbonyl]-5-cyclopropyl-1H-pyrazol-3-yl}-3-hydroxyphenyl)isonicotinamide;
Ir. 3-{4-[(1,3-benzodioxol-5-ylcarbonyl)amino]-2-hydroxyphenyl}-N-butyl-5-cyclobutyl-1H-pyrazole-1-carboxamide;
Is. 3-{4-[(1,3-benzodioxol-5-ylcarbonyl)amino]-2-hydroxyphenyl}-N-butyl-5-cyclopentyl-1H-pyrazole-1-carboxamide;
It. N-allyl-5-cyclopropyl-3-(4-fluoro-2-hydroxyphenyl)-1H-pyrazole-1-carboxamide;
Iu. 5-(3,4-dimethoxyphenyl)-N-ethyl-3-(2-hydroxy-5-methoxyphenyl)-1H-pyrazole-1-carboxamide;
Iv. N-allyl-5-[2-(dimethylamino)ethyl]-3-(2-hydroxy-5-methylphenyl)-1H-pyrazole-1-carboxamide;
Iw. 5-[2-(dimethylamino)ethyl]-3-(2-hydroxy-5-methoxyphenyl)-N-(2-phenylethyl)-1H-pyrazole-1-carboxamide;
Ix. ethyl-N-{[5-(3,4-dimethoxyphenyl)-3-(2-hydroxy-5-methoxyphenyl)-1H-pyrazol-1-yl]carbonyl}glycinate;
Iy. 5-cyclopropyl-N-ethyl-3-(4-fluoro-2-hydroxyphenyl)-1H-pyrazole-1-carboxamide
Iz. ethyl-N-{[3-(5-chloro-2-hydroxyphenyl)-5-cyclopropyl-1H-pyrazol-1-yl]carbonyl}glycinate;
Iaa. 5-cyclopropyl-3-(2-hydroxy-5-methoxyphenyl)-N-(2-phenylethyl)-1H-pyrazole-1-carboxamide;
Ibb. 5-cyclopropyl-N-ethyl-3-(2-hydroxyphenyl)-1H-pyrazole-1-carboxamide;
Icc. N-allyl-5-(3,4-dimethoxyphenyl)-3-(2-hydroxy-5-methoxyphenyl)-1H-pyrazole-1-carboxamide;
Idd. N-benzyl-5-(3,4-dimethoxyphenyl)-3-(2-hydroxy-5-methylphenyl)-1H-pyrazole-1-carboxamide;
Iee. N-allyl-5-cyclopropyl-3-(2-hydroxy-5-methoxyphenyl)-1H-pyrazole-1-carboxamide;
Iff. N-benzyl-5-[2-(dimethylamino)ethyl]-3-(2-hydroxy-5-methoxyphenyl)-1H-pyrazole-1-carboxamide;
Igg. 5-cyclopropyl-3-(2-hydroxy-5-methylphenyl)-N-(2-phenylethyl)-1H-pyrazole-1-carboxamide;
Ihh. 5-[2-(dimethylamino)ethyl]-N-ethyl-3-(2-hydroxyphenyl)-1H-pyrazole-1-carboxamide;
Iii. ethyl N-[(5-cyclobutyl-3-{2-hydroxy-4-[(3-methoxybenzoyl)amino]phenyl}-1H-pyrazol-1-yl)carbonyl]glycinate and
Ijj. ethyl N-[(5-cyclopropyl-3-{2-hydroxy-4-[(3-methoxybenzoyl)amino]phenyl}-1H-azol-1-yl)carbonyl]glycinate, or a pharmaceutically acceptable salt thereof.

20. A compound of formula (I) according to claim 12 which is selected from:

| | |
|---|---|
| 1 | 3-{4-[(2-chlorobenzoyl)amino]-2-hydroxyphenyl}-5-cyclopropyl-N-isopropyl- |
| | 1H-pyrazole-1-carboxamide |
| 2 | 3-{4-[(2-chlorobenzoyl)amino]-2-hydroxyphenyl}-5-cyclopropyl-N-ethyl-1H- |
| | pyrazole-1-carboxamide |
| 3 | N-allyl-3-{4-[(2-chlorobenzoyl)amino]-2-hydroxyphenyl}-5-cyclopropyl-1H- |
| | pyrazole-1-carboxamide |
| 4 | 3-{4-[(2-chlorobenzoyl)amino]-2-hydroxyphenyl}-5-cyclopropyl-N-propyl-1H- |
| | pyrazole-1-carboxamide |
| 5 | N-benzyl-3-{4-[(2-chlorobenzoyl)amino]-2-hydroxyphenyl}-5-cyclopropyl-1H- |
| | pyrazole-1-carboxamide |
| 6 | 3-{4-[(2-chlorobenzoyl)amino]-2-hydroxyphenyl}-5-cyclopropyl-N-(2- |
| | phenylethyl)-1H-pyrazole-1-carboxamide |
| 7 | 3-{4-[(2-chlorobenzoyl)amino]-2-hydroxyphenyl}-5-cyclopropyl-N-(2- |
| | methylbenzyl)-1H-pyrazole-1-carboxamide |
| 8 | 3-{4-[(2-chlorobenzoyl)amino]-2-hydroxyphenyl}-5-cyclopropyl-N-(3- |
| | methylbenzyl)-1H-pyrazole-1-carboxamide |
| 9 | 3-{4-[(2-chlorobenzoyl)amino]-2-hydroxyphenyl}-5-cyclopropyl-N-(4- |
| | methylbenzyl)-1H-pyrazole-1-carboxamide |
| 10 | 3-{4-[(2-chlorobenzoyl)amino]-2-hydroxyphenyl}-N-(2-chlorobenzyl)-5- |
| | cyclopropyl-1H-pyrazole-1-carboxamide |
| 11 | 3-{4-[(2-chlorobenzoyl)amino]-2-hydroxyphenyl}-5-cyclopropyl-N-(4- |
| | fluorobenzyl)-1H-pyrazole-1-carboxamide |
| 12 | 3-{4-[(2-chlorobenzoyl)amino]-2-hydroxyphenyl}-5-cyclopropyl-N-(3,4- |
| | dichlorobenzyl)-1H-pyrazole-1-carboxamide |
| 13 | 3-{4-[(2-chlorobenzoyl)amino]-2-hydroxyphenyl}-5-cyclopropyl-N-(4- |
| | methoxybenzyl)-1H-pyrazole-1-carboxamide |
| 14 | N-(sec-butyl)-3-{4-[(2-chlorobenzoyl)amino]-2-hydroxyphenyl}-5-cyclopropyl- |
| | 1H-pyrazole-1-carboxamide |
| 15 | 3-{4-[(2-chlorobenzoyl)amino]-2-hydroxyphenyl}-5-cyclopropyl-N-(2- |
| | fluorobenzyl)-1H-pyrazole-1-carboxamide |
| 16 | 3-{4-[(2-chlorobenzoyl)amino]-2-hydroxyphenyl}-5-cyclopropyl-N-(3- |
| | fluorobenzyl)-1H-pyrazole-1-carboxamide |
| 17 | 3-{4-[(2-chlorobenzoyl)amino]-2-hydroxyphenyl}-N,5-dicyclopropyl-1H- |
| | pyrazole-1-carboxamide |
| 18 | 3-{4-[(2-chlorobenzoyl)amino]-2-hydroxyphenyl}-5-cyclobutyl-N-isopropyl-1H- |
| | pyrazole-1-carboxamide |
| 19 | 3-{4-[(2-chlorobenzoyl)amino]-2-hydroxyphenyl}-5-cyclobutyl-N-ethyl-1H- |
| | pyrazole-1-carboxamide |
| 20 | N-allyl-3-{4-[(2-chlorobenzoyl)amino]-2-hydroxyphenyl}-5-cyclobutyl-1H- |
| | pyrazole-1-carboxamide |
| 21 | 3-{4-[(2-chlorobenzoyl)amino]-2-hydroxyphenyl}-5-cyclobutyl-N-propyl-1H- |
| | pyrazole-1-carboxamide |
| 22 | N-benzyl-3-{4-[(2-chlorobenzoyl)amino]-2-hydroxyphenyl}-5-cyclobutyl-1H- |
| | pyrazole-1-carboxamide |
| 23 | 3-{4-[(2-chlorobenzoyl)amino]-2-hydroxyphenyl}-5-cyclobutyl-N-(2- |
| | phenylethyl)-1H-pyrazole-1-carboxamide |
| 24 | 3-{4-[(2-chlorobenzoyl)amino]-2-hydroxyphenyl}-5-cyclobutyl-N-(2- |
| | methylbenzyl)-1H-pyrazole-1-carboxamide |
| 25 | 3-{4-[(2-chlorobenzoyl)amino]-2-hydroxyphenyl}-5-cyclobutyl-N-(3- |
| | methylbenzyl)-1H-pyrazole-1-carboxamide |
| 26 | 3-{4-[(2-chlorobenzoyl)amino]-2-hydroxyphenyl}-5-cyclobutyl-N-(4- |
| | methylbenzyl)-1H-pyrazole-1-carboxamide |
| 27 | 3-{4-[(2-chlorobenzoyl)amino]-2-hydroxyphenyl}-N-(2-chlorobenzyl)-5- |
| | cyclobutyl-1H-pyrazole-1-carboxamide |
| 28 | 3-{4-[(2-chlorobenzoyl)amino]-2-hydroxyphenyl}-5-cyclobutyl-N-(4- |
| | fluorobenzyl)-1H-pyrazole-1-carboxamide |
| 29 | 3-{4-[(2-chlorobenzoyl)amino]-2-hydroxyphenyl}-5-cyclobutyl-N-(3,4- |
| | dichlorobenzyl)-1H-pyrazole-1-carboxamide |
| 30 | 3-{4-[(2-chlorobenzoyl)amino]-2-hydroxyphenyl}-5-cyclobutyl-N-(4- |
| | methoxybenzyl)-1H-pyrazole-1-carboxamide |
| 31 | N-(sec-butyl)-3-{4-[(2-chlorobenzoyl)amino]-2-hydroxyphenyl}-5-cyclobutyl- |
| | 1H-pyrazole-1-carboxamide |
| 32 | 3-{4-[(2-chlorobenzoyl)amino]-2-hydroxyphenyl}-5-cyclobutyl-N-(2- |
| | fluorobenzyl)-1H-pyrazole-1-carboxamide |
| 33 | 3-{4-[(2-chlorobenzoyl)amino]-2-hydroxyphenyl}-5-cyclobutyl-N-(3- |
| | fluorobenzyl)-1H-pyrazole-1-carboxamide |
| 34 | 3-{4-[(2-chlorobenzoyl)amino]-2-hydroxyphenyl}-5-cyclobutyl-N-cyclopropyl- |
| | 1H-pyrazole-1-carboxamide |
| 35 | 3-{4-[(2-chlorobenzoyl)amino]-2-hydroxyphenyl}-5-cyclopentyl-N-isopropyl- |
| | 1H-pyrazole-1-carboxamide |
| 36 | 3-{4-[(2-chlorobenzoyl)amino]-2-hydroxyphenyl}-5-cyclopentyl-N-ethyl-1H- |
| | pyrazole-1-carboxamide |
| 37 | N-allyl-3-{4-[(2-chlorobenzoyl)amino]-2-hydroxyphenyl}-5-cyclopentyl-1H- |
| | pyrazole-1-carboxamide |
| 38 | 3-{4-[(2-chlorobenzoyl)amino]-2-hydroxyphenyl}-5-cyclopentyl-N-propyl-1H- |
| | pyrazole-1-carboxamide |
| 39 | N-benzyl-3-{4-[(2-chlorobenzoyl)amino]-2-hydroxyphenyl}-5-cyclopentyl-1H- |
| | pyrazole-1-carboxamide |
| 40 | 3-{4-[(2-chlorobenzoyl)amino]-2-hydroxyphenyl}-5-cyclopentyl-N-(2- |
| | phenylethyl)-1H-pyrazole-1-carboxamide |
| 41 | 3-{4-[(2-chlorobenzoyl)amino]-2-hydroxyphenyl}-5-cyclopentyl-N-(2- |
| | methylbenzyl)-1H-pyrazole-1-carboxamide |
| 42 | 3-{4-[(2-chlorobenzoyl)amino]-2-hydroxyphenyl}-5-cyclopentyl-N-(3- |
| | methylbenzyl)-1H-pyrazole-1-carboxamide |
| 43 | 3-{4-[(2-chlorobenzoyl)amino]-2-hydroxyphenyl}-5-cyclopentyl-N-(4- |
| | methylbenzyl)-1H-pyrazole-1-carboxamide |
| 44 | 3-{4-[(2-chlorobenzoyl)amino]-2-hydroxyphenyl}-N-(2-chlorobenzyl)-5- |
| | cyclopentyl-1H-pyrazole-1-carboxamide |
| 45 | 3-{4-[(2-chlorobenzoyl)amino]-2-hydroxyphenyl}-5-cyclopentyl-N-(4- |
| | fluorobenzyl)-1H-pyrazole-1-carboxamide |
| 46 | 5-cyclopropyl-3-{4-[(2,6-dichlorobenzoyl)amino]-2-hydroxyphenyl}-N- |
| | isopropyl-1H-pyrazole-1-carboxamide |
| 47 | 3-{4-[(2-chlorobenzoyl)amino]-2-hydroxyphenyl}-5-cyclopentyl-N-(4- |
| | methoxybenzyl)-1H-pyrazole-1-carboxamide |
| 48 | N-(sec-butyl)-3-{4-[(2-chlorobenzoyl)amino]-2-hydroxyphenyl}-5-cyclopentyl- |
| | 1H-pyrazole-1-carboxamide |
| 49 | 3-{4-[(2-chlorobenzoyl)amino]-2-hydroxyphenyl}-5-cyclopentyl-N-(2- |
| | fluorobenzyl)-1H-pyrazole-1-carboxamide |
| 50 | 3-{4-[(2-chlorobenzoyl)amino]-2-hydroxyphenyl}-5-cyclopentyl-N-(3- |
| | fluorobenzyl)-1H-pyrazole-1-carboxamide |
| 51 | 3-{4-[(2-chlorobenzoyl)amino]-2-hydroxyphenyl}-5-cyclopentyl-N-cyclopropyl- |
| | 1H-pyrazole-1-carboxamide |
| 52 | 5-cyclopropyl-3-{4-[(2,6-dichlorobenzoyl)amino]-2-hydroxyphenyl}-N- |
| | isopropyl-1H-pyrazole-1-carboxamide |
| 53 | 5-cyclopropyl-3-{4-[(2,6-dichlorobenzoyl)amino]-2-hydroxyphenyl}-N-ethyl-1H- |
| | pyrazole-1-carboxamide |
| 54 | N-allyl-5-cyclopropyl-3-{4-[(2,6-dichlorobenzoyl)amino]-2-hydroxyphenyl}-1H- |
| | pyrazole-1-carboxamide |
| 55 | 5-cyclopropyl-3-{4-[(2,6-dichlorobenzoyl)amino]-2-hydroxyphenyl}-N-propyl- |
| | 1H-pyrazole-1-carboxamide |
| 56 | N-benzyl-5-cyclopropyl-3-{4-[(2,6-dichlorobenzoyl)amino]-2-hydroxyphenyl}- |
| | 1H-pyrazole-1-carboxamide |
| 57 | 5-cyclopropyl-3-{4-[(2,6-dichlorobenzoyl)amino]-2-hydroxyphenyl}-N-(2- |
| | phenylethyl)-1H-pyrazole-1-carboxamide |
| 58 | 5-cyclopropyl-3-{4-[(2,6-dichlorobenzoyl)amino]-2-hydroxyphenyl}-N-(2- |
| | methylbenzyl)-1H-pyrazole-1-carboxamide |
| 59 | 5-cyclopropyl-3-{4-[(2,6-dichlorobenzoyl)amino]-2-hydroxyphenyl}-N-(3,4- |
| | dichlorobenzyl)-1H-pyrazole-1-carboxamide |
| 60 | 5-cyclopropyl-3-{4-[(2,6-dichlorobenzoyl)amino]-2-hydroxyphenyl}-N-(4- |
| | methylbenzyl)-1H-pyrazole-1-carboxamide |
| 61 | N-(2-chlorobenzyl)-5-cyclopropyl-3-{4-[(2,6-dichlorobenzoyl)amino]-2- |
| | hydroxyphenyl}-1H-pyrazole-1-carboxamide |
| 62 | 5-cyclopropyl-3-{4-[(2,6-dichlorobenzoyl)amino]-2-hydroxyphenyl}-N-(4- |
| | fluorobenzyl)-1H-pyrazole-1-carboxamide |
| 63 | 5-cyclopropyl-3-{4-[(2,6-dichlorobenzoyl)amino]-2-hydroxyphenyl}-N-(3,4- |
| | dichlorobenzyl)-1H-pyrazole-1-carboxamide |
| 64 | 5-cyclopropyl-3-{4-[(2,6-dichlorobenzoyl)amino]-2-hydroxyphenyl}-N-(4- |
| | methoxybenzyl)-1H-pyrazole-1-carboxamide |
| 65 | N-(sec-butyl)-5-cyclopropyl-3-{4-[(2,6-dichlorobenzoyl)amino]-2- |
| | hydroxyphenyl}-1H-pyrazole-1-carboxamide |
| 66 | 5-cyclopropyl-3-{4-[(2,6-dichlorobenzoyl)amino]-2-hydroxyphenyl}-N-(2- |
| | fluorobenzyl)-1H-pyrazole-1-carboxamide |
| 67 | 5-cyclopropyl-3-{4-[(2,6-dichlorobenzoyl)amino]-2-hydroxyphenyl}-N-(3- |
| | fluorobenzyl)-1H-pyrazole-1-carboxamide |
| 68 | N,5-dicyclopropyl-3-{4-[(2,6-dichlorobenzoyl)amino]-2-hydroxyphenyl}-1H- |
| | pyrazole-1-carboxamide |
| 69 | 5-cyclobutyl-3-{4-[(2,6-dichlorobenzoyl)amino]-2-hydroxyphenyl}-N-isopropyl- |
| | 1H-pyrazole-1-carboxamide |
| 70 | 5-cyclobutyl-3-{4-[(2,6-dichlorobenzoyl)amino]-2-hydroxyphenyl}-N-ethyl-1H- |
| | pyrazole-1-carboxamide |
| 71 | N-allyl-5-cyclobutyl-3-{4-[(2,6-dichlorobenzoyl)amino]-2-hydroxyphenyl}-1H- |
| | pyrazole-1-carboxamide |
| 72 | 5-cyclobutyl-3-{4-[(2,6-dichlorobenzoyl)amino]-2-hydroxyphenyl}-N-propyl-1H- |
| | pyrazole-1-carboxamide |
| 73 | N-benzyl-5-cyclobutyl-3-{4-[(2,6-dichlorobenzoyl)amino]-2-hydroxyphenyl}- |
| | 1H-pyrazole-1-carboxamide |
| 74 | 5-cyclobutyl-3-{4-[(2,6-dichlorobenzoyl)amino]-2-hydroxyphenyl}-N-(2- |
| | phenylethyl)-1H-pyrazole-1-carboxamide |
| 75 | 5-cyclobutyl-3-{4-[(2,6-dichlorobenzoyl)amino]-2-hydroxyphenyl}-N-(2- |
| | methylbenzyl)-1H-pyrazole-1-carboxamide |
| 76 | 5-cyclobutyl-3-{4-[(2,6-dichlorobenzoyl)amino]-2-hydroxyphenyl}-N-(3- |
| | methylbenzyl)-1H-pyrazole-1-carboxamide |
| 77 | 5-cyclobutyl-3-{4-[(2,6-dichlorobenzoyl)amino]-2-hydroxyphenyl}-N-(4- |
| | methylbenzyl)-1H-pyrazole-1-carboxamide |
| 78 | N-(2-chlorobenzyl)-5-cyclobutyl-3-{4-[(2,6-dichlorobenzoyl)amino]-2- |
| | hydroxyphenyl}-1H-pyrazole-1-carboxamide |
| 79 | 5-cyclobutyl-3-{4-[(2,6-dichlorobenzoyl)amino]-2-hydroxyphenyl}-N-(4- |
| | fluorobenzyl)-1H-pyrazole-1-carboxamide |
| 80 | 5-cyclobutyl-3-{4-[(2,6-dichlorobenzoyl)amino]-2-hydroxyphenyl}-N-(3,4- |
| | dichlorobenzyl)-1H-pyrazole-1-carboxamide |
| 81 | 5-cyclobutyl-3-{4-[(2,6-dichlorobenzoyl)amino]-2-hydroxyphenyl}-N-(4- |
| | methoxybenzyl)-1H-pyrazole-1-carboxamide |
| 82 | N-(sec-butyl)-5-cyclobutyl-3-{4-[(2,6-dichlorobenzoyl)amino]-2- |
| | hydroxyphenyl}-1H-pyrazole-1-carboxamide |
| 83 | 5-cyclobutyl-3-{4-[(2,6-dichlorobenzoyl)amino]-2-hydroxyphenyl}-N-(2- |
| | fluorobenzyl)-1H-pyrazole-1-carboxamide |
| 84 | 5-cyclobutyl-3-{4-[(2,6-dichlorobenzoyl)amino]-2-hydroxyphenyl}-N-(3- |
| | fluorobenzyl)-1H-pyrazole-1-carboxamide |
| 85 | 5-cyclobutyl-N-cyclopropyl-3-{4-[(2,6-dichlorobenzoyl)amino]-2- |
| | hydroxyphenyl}-1H-pyrazole-1-carboxamide |
| 86 | 5-cyclopentyl-3-{4-[(2,6-dichlorobenzoyl)amino]-2-hydroxyphenyl}-N- |
| | isopropyl-1H-pyrazole-1-carboxamide |
| 87 | 5-cyclopentyl-3-{4-[(2,6-dichlorobenzoyl)amino]-2-hydroxyphenyl}-N-ethyl-1H- |
| | pyrazole-1-carboxamide |
| 88 | N-allyl-5-cyclopentyl-3-{4-[(2,6-dichlorobenzoyl)amino]-2-hydroxyphenyl}-1H- |
| | pyrazole-1-carboxamide |
| 89 | 5-cyclopentyl-3-{4-[(2,6-dichlorobenzoyl)amino]-2-hydroxyphenyl}-N-propyl- |
| | 1H-pyrazole-1-carboxamide |
| 90 | N-benzyl-5-cyclopentyl-3-{4-[(2,6-dichlorobenzoyl)amino]-2-hydroxyphenyl}- |
| | 1H-pyrazole-1-carboxamide |
| 91 | 5-cyclopentyl-3-{4-[(2,6-dichlorobenzoyl)amino]-2-hydroxyphenyl}-N-(2- |
| | phenylethyl)-1H-pyrazole-1-carboxamide |
| 92 | 5-cyclopentyl-3-{4-[(2,6-dichlorobenzoyl)amino]-2-hydroxyphenyl}-N-(2- |
| | methylbenzyl)-1H-pyrazole-1-carboxamide |
| 93 | 5-cyclopentyl-3-{4-[(2,6-dichlorobenzoyl)amino]-2-hydroxyphenyl}-N-(3- |
| | methylbenzyl)-1H-pyrazole-1-carboxamide |
| 94 | 5-cyclopentyl-3-{4-[(2,6-dichlorobenzoyl)amino]-2-hydroxyphenyl}-N-(4- |
| | methylbenzyl)-1H-pyrazole-1-carboxamide |
| 95 | N-(2-chlorobenzyl)-5-cyclopentyl-3-{4-[(2,6-dichlorobenzoyl)amino]-2- |
| | hydroxyphenyl}-1H-pyrazole-1-carboxamide |
| 96 | 5-cyclopentyl-3-{4-[(2,6-dichlorobenzoyl)amino]-2-hydroxyphenyl}-N-(4- |
| | fluorobenzyl)-1H-pyrazole-1-carboxamide |
| 97 | 5-cyclopentyl-3-{4-[(2,6-dichlorobenzoyl)amino]-2-hydroxyphenyl}-N-(3,4- |
| | dichlorobenzyl)-1H-pyrazole-1-carboxamide |
| 98 | 5-cyclopentyl-3-{4-[(2,6-dichlorobenzoyl)amino]-2-hydroxyphenyl}-N-(4- |
| | methoxybenzyl)-1H-pyrazole-1-carboxamide |
| 99 | N-(sec-butyl)-5-cyclopentyl-3-{4-[(2,6-dichlorobenzoyl)amino]-2- |
| | hydroxyphenyl}-1H-pyrazole-1-carboxamide |
| 100 | 5-cyclopentyl-3-{4-[(2,6-dichlorobenzoyl)amino]-2-hydroxyphenyl}-N-(2- |
| | fluorobenzyl)-1H-pyrazole-1-carboxamide |
| 101 | 5-cyclopentyl-3-{4-[(2,6-dichlorobenzoyl)amino]-2-hydroxyphenyl}-N-(3- |
| | fluorobenzyl)-1H-pyrazole-1-carboxamide |
| 102 | 5-cyclopentyl-N-cyclopropyl-3-{4-[(2,6-dichlorobenzoyl)amino]-2- |
| | hydroxyphenyl}-1H-pyrazole-1-carboxamide |
| 103 | 5-cyclopropyl-3-{2-hydroxy-4-[(2-methoxybenzoyl)amino]phenyl}-N-isopropyl- |
| | 1H-pyrazole-1-carboxamide |
| 104 | 5-cyclopropyl-N-ethyl-3-{2-hydroxy-4-[(2-methoxybenzoyl)amino]phenyl}-1H- |
| | pyrazole-1-carboxamide |
| 105 | N-allyl-5-cyclopropyl-3-{2-hydroxy-4-[(2-methoxybenzoyl)amino]phenyl}-1H- |
| | pyrazole-1-carboxamide |
| 106 | 5-cyclopropyl-3-{2-hydroxy-4-[(2-methoxybenzoyl)amino]phenyl}-N-propyl-1H- |
| | pyrazole-1-carboxamide |
| 107 | N-benzyl-5-cyclopropyl-3-{2-hydroxy-4-[(2-methoxybenzoyl)amino]phenyl}- |
| | 1H-pyrazole-1-carboxamide |
| 108 | 5-cyclopropyl-3-{2-hydroxy-4-[(2-methoxybenzoyl)amino]phenyl}-N-(2- |
| | phenylethyl)-1H-pyrazole-1-carboxamide |
| 109 | 5-cyclopropyl-3-{2-hydroxy-4-[(2-methoxybenzoyl)amino]phenyl}-N-(2- |
| | methylbenzyl)-1H-pyrazole-1-carboxamide |
| 110 | 5-cyclopropyl-3-{2-hydroxy-4-[(2-methoxybenzoyl)amino]phenyl}-N-(3- |
| | methylbenzyl)-1H-pyrazole-1-carboxamide |
| 111 | 5-cyclopropyl-3-{2-hydroxy-4-[(2-methoxybenzoyl)amino]phenyl}-N-(4- |
| | methylbenzyl)-1H-pyrazole-1-carboxamide |
| 112 | N-(2-chlorobenzyl)-5-cyclopropyl-3-{2-hydroxy-4-[(2- |
| | methoxybenzoyl)amino]phenyl}-1H-pyrazole-1-carboxamide |
| 113 | 5-cyclopropyl-N-(4-fluorobenzyl)-3-{2-hydroxy-4-[(2- |
| | methoxybenzoyl)amino]phenyl}-1H-pyrazole-1-carboxamide |
| 114 | 5-cyclopropyl-N-(3,4-dichlorobenzyl)-3-{2-hydroxy-4-[(2- |
| | methoxybenzoyl)amino]phenyl}-1H-pyrazole-1-carboxamide |
| 115 | 5-cyclopropyl-3-{2-hydroxy-4-[(2-methoxybenzoyl)amino]phenyl}-N-(4- |
| | methoxybenzyl)-1H-pyrazole-1-carboxamide |
| 116 | N-(sec-butyl)-5-cyclopropyl-3-{2-hydroxy-4-[(2- |
| | methoxybenzoyl)amino]phenyl}-1H-pyrazole-1-carboxamide |
| 117 | 5-cyclopropyl-N-(2-fluorobenzyl)-3-{2-hydroxy-4-[(2- |
| | methoxybenzoyl)amino]phenyl}-1H-pyrazole-1-carboxamide |
| 118 | 5-cyclopropyl-N-(3-fluorobenzyl)-3-{2-hydroxy-4-[(2- |
| | methoxybenzoyl)amino]phenyl}-1H-pyrazole-1-carboxamide |
| 119 | N,5-dicyclopropyl-3-{2-hydroxy-4-[(2-methoxybenzoyl)amino]phenyl}-1H- |
| | pyrazole-1-carboxamide |
| 120 | 5-cyclobutyl-3-{2-hydroxy-4-[(2-methoxybenzoyl)amino]phenyl}-N-isopropyl- |
| | 1H-pyrazole-1-carboxamide |
| 121 | 5-cyclobutyl-N-ethyl-3-{2-hydroxy-4-[(2-methoxybenzoyl)amino]phenyl}-1H- |
| | pyrazole-1-carboxamide |
| 122 | N-allyl-5-cyclobutyl-3-{2-hydroxy-4-[(2-methoxybenzoyl)amino]phenyl}-1H- |
| | pyrazole-1-carboxamide |
| 123 | 5-cyclobutyl-3-{2-hydroxy-4-[(2-methoxybenzoyl)amino]phenyl}-N-propyl-1H- |
| | pyrazole-1-carboxamide |
| 124 | N-benzyl-5-cyclobutyl-3-{2-hydroxy-4-[(2-methoxybenzoyl)amino]phenyl}-1H- |
| | pyrazole-1-carboxamide |
| 125 | 5-cyclobutyl-3-{2-hydroxy-4-[(2-methoxybenzoyl)amino]phenyl}-N-(2- |
| | phenylethyl)-1H-pyrazole-1-carboxamide |
| 126 | 5-cyclobutyl-3-{2-hydroxy-4-[(2-methoxybenzoyl)amino]phenyl}-N-(2- |
| | methylbenzyl)-1H-pyrazole-1-carboxamide |
| 127 | 5-cyclobutyl-3-{2-hydroxy-4-[(2-methoxybenzoyl)amino]phenyl}-N-(3- |
| | methylbenzyl)-1H-pyrazole-1-carboxamide |
| 128 | 5-cyclobutyl-3-{2-hydroxy-4-[(2-methoxybenzoyl)amino]phenyl}-N-(4- |
| | methylbenzyl)-1H-pyrazole-1-carboxamide |
| 129 | N-(2-chlorobenzyl)-5-cyclobutyl-3-{2-hydroxy-4-[(2- |
| | methoxybenzoyl)amino]phenyl}-1H-pyrazole-1-carboxamide |
| 130 | 5-cyclobutyl-N-(4-fluorobenzyl)-3-{2-hydroxy-4-[(2- |
| | methoxybenzoyl)amino]phenyl}-1H-pyrazole-1-carboxamide |
| 131 | 5-cyclobutyl-N-(3,4-dichlorobenzyl)-3-{2-hydroxy-4-[(2- |
| | methoxybenzoyl)amino]phenyl}-1H-pyrazole-1-carboxamide |
| 132 | 5-cyclobutyl-3-{2-hydroxy-4-[(2-methoxybenzoyl)amino]phenyl}-N-(4- |
| | methoxybenzyl)-1H-pyrazole-1-carboxamide |
| 133 | N-(sec-butyl)-5-cyclobutyl-3-{2-hydroxy-4-[(2-methoxybenzoyl)amino]phenyl}- |
| | 1H-pyrazole-1-carboxamide |
| 134 | 5-cyclobutyl-N-(2-fluorobenzyl)-3-{2-hydroxy-4-[(2- |
| | methoxybenzoyl)amino]phenyl}-1H-pyrazole-1-carboxamide |
| 135 | 5-cyclobutyl-N-(3-fluorobenzyl)-3-{2-hydroxy-4-[(2- |
| | methoxybenzoyl)amino]phenyl}-1H-pyrazole-1-carboxamide |
| 136 | 5-cyclobutyl-N-cyclopropyl-3-{2-hydroxy-4-[(2- |
| | methoxybenzoyl)amino]phenyl}-1H-pyrazole-1-carboxamide |
| 137 | 5-cyclopentyl-3-{2-hydroxy-4-[(2-methoxybenzoyl)amino]phenyl}-N-isopropyl- |
| | 1H-pyrazole-1-carboxamide |
| 138 | 5-cyclopentyl-N-ethyl-3-{2-hydroxy-4-[(2-methoxybenzoyl)amino]phenyl}-1H- |
| | pyrazole-1-carboxamide |
| 139 | N-allyl-5-cyclopentyl-3-{2-hydroxy-4-[(2-methoxybenzoyl)amino]phenyl}-1H- |
| | pyrazole-1-carboxamide |
| 140 | 5-cyclopentyl-3-{2-hydroxy-4-[(2-methoxybenzoyl)amino]phenyl}-N-propyl-1H- |
| | pyrazole-1-carboxamide |
| 141 | N-benzyl-5-cyclopentyl-3-{2-hydroxy-4-[(2-methoxybenzoyl)amino]phenyl}-1H- |
| | pyrazole-1-carboxamide |
| 142 | 5-cyclopentyl-3-{2-hydroxy-4-[(2-methoxybenzoyl)amino]phenyl}-N-(2- |
| | phenylethyl)-1H-pyrazole-1-carboxamide |
| 143 | 5-cyclopentyl-3-{2-hydroxy-4-[(2-methoxybenzoyl)amino]phenyl}-N-(2- |
| | methylbenzyl)-1H-pyrazole-1-carboxamide |
| 144 | 5-cyclopentyl-3-{2-hydroxy-4-[(2-methoxybenzoyl)amino]phenyl}-N-(3- |
| | methylbenzyl)-1H-pyrazole-1-carboxamide |
| 145 | 5-cyclopentyl-3-{2-hydroxy-4-[(2-methoxybenzoyl)amino]phenyl}-N-(4- |
| | methylbenzyl)-1H-pyrazole-1-carboxamide |
| 146 | N-(2-chlorobenzyl)-5-cyclopentyl-3-{2-hydroxy-4-[(2- |
| | methoxybenzoyl)amino]phenyl}-1H-pyrazole-1-carboxamide |
| 147 | 5-cyclopentyl-N-(4-fluorobenzyl)-3-{2-hydroxy-4-[(2- |
| | methoxybenzoyl)amino]phenyl}-1H-pyrazole-1-carboxamide |
| 148 | 5-cyclopentyl-N-(3,4-dichlorobenzyl)-3-{2-hydroxy-4-[(2- |
| | methoxybenzoyl)amino]phenyl}-1H-pyrazole-1-carboxamide |
| 149 | 5-cyclopentyl-3-{2-hydroxy-4-[(2-methoxybenzoyl)amino]phenyl}-N-(4- |
| | methoxybenzyl)-1H-pyrazole-1-carboxamide |
| 150 | N-(sec-butyl)-5-cyclopentyl-3-{2-hydroxy-4-[(2- |
| | methoxybenzoyl)amino]phenyl}-1H-pyrazole-1-carboxamide |
| 151 | 5-cyclopentyl-N-(2-fluorobenzyl)-3-{2-hydroxy-4-[(2- |
| | methoxybenzoyl)amino]phenyl}-1H-pyrazole-1-carboxamide |
| 152 | 5-cyclopentyl-N-(3-fluorobenzyl)-3-{2-hydroxy-4-[(2- |
| | methoxybenzoyl)amino]phenyl}-1H-pyrazole-1-carboxamide |
| 153 | 5-cyclopentyl-N-cyclopropyl-3-{2-hydroxy-4-[(2- |
| | methoxybenzoyl)amino]phenyl}-1H-pyrazole-1-carboxamide |
| 154 | 5-cyclopropyl-3-{2-hydroxy-4-[(2-methylbenzoyl)amino]phenyl}-N-isopropyl- |
| | 1H-pyrazole-1-carboxamide |
| 155 | 5-cyclopropyl-N-ethyl-3-{2-hydroxy-4-[(2-methylbenzoyl)amino]phenyl}-1H- |
| | pyrazole-1-carboxamide |
| 156 | N-allyl-5-cyclopropyl-3-{2-hydroxy-4-[(2-methylbenzoyl)amino]phenyl}-1H- |
| | pyrazole-1-carboxamide |
| 157 | 5-cyclopropyl-3-{2-hydroxy-4-[(2-methylbenzoyl)amino]phenyl}-N-propyl-1H- |
| | pyrazole-1-carboxamide |
| 158 | N-benzyl-5-cyclopropyl-3-{2-hydroxy-4-[(2-methylbenzoyl)amino]phenyl}-1H- |
| | pyrazole-1-carboxamide |
| 159 | 5-cyclopropyl-3-{2-hydroxy-4-[(2-methylbenzoyl)amino]phenyl}-N-(2- |
| | phenylethyl)-1H-pyrazole-1-carboxamide |
| 160 | 5-cyclopropyl-3-{2-hydroxy-4-[(2-methylbenzoyl)amino]phenyl}-N-(2- |
| | methylbenzyl)-1H-pyrazole-1-carboxamide |
| 161 | 5-cyclopropyl-3-{2-hydroxy-4-[(2-methylbenzoyl)amino]phenyl}-N-(3- |
| | methylbenzyl)-1H-pyrazole-1-carboxamide |
| 162 | 5-cyclopropyl-3-{2-hydroxy-4-[(2-methylbenzoyl)amino]phenyl}-N-(4- |
| | methylbenzyl)-1H-pyrazole-1-carboxamide |
| 163 | N-(2-chlorobenzyl)-5-cyclopropyl-3-{2-hydroxy-4-[(2- |
| | methylbenzoyl)amino]phenyl}-1H-pyrazole-1-carboxamide |
| 164 | 5-cyclopropyl-N-(4-fluorobenzyl)-3-{2-hydroxy-4-[(2- |
| | methylbenzoyl)amino]phenyl}-1H-pyrazole-1-carboxamide |
| 165 | 5-cyclopropyl-N-(3,4-dichlorobenzyl)-3-{2-hydroxy-4-[(2- |
| | methylbenzoyl)amino]phenyl}-1H-pyrazole-1-carboxamide |
| 166 | 5-cyclopropyl-3-{2-hydroxy-4-[(2-methylbenzoyl)amino]phenyl}-N-(4- |
| | methoxybenzyl)-1H-pyrazole-1-carboxamide |
| 167 | N-(sec-butyl)-5-cyclopropyl-3-{2-hydroxy-4-[(2-methylbenzoyl)amino]phenyl}- |
| | 1H-pyrazole-1-carboxamide |
| 168 | 5-cyclopropyl-N-(2-fluorobenzyl)-3-{2-hydroxy-4-[(2- |
| | methylbenzoyl)amino]phenyl}-1H-pyrazole-1-carboxamide |
| 169 | 5-cyclopropyl-N-(3-fluorobenzyl)-3-{2-hydroxy-4-[(2- |
| | methylbenzoyl)amino]phenyl}-1H-pyrazole-1-carboxamide |
| 170 | N,5-dicyclopropyl-3-{2-hydroxy-4-[(2-methylbenzoyl)amino]phenyl}-1H- |
| | pyrazole-1-carboxamide |
| 171 | 5-cyclobutyl-3-{2-hydroxy-4-[(2-methylbenzoyl)amino]phenyl}-N-isopropyl-1H- |
| | pyrazole-1-carboxamide |
| 172 | 5-cyclobutyl-N-ethyl-3-{2-hydroxy-4-[(2-methylbenzoyl)amino]phenyl}-1H- |
| | pyrazole-1-carboxamide |
| 173 | N-allyl-5-cyclobutyl-3-{2-hydroxy-4-[(2-methylbenzoyl)amino]phenyl}-1H- |
| | pyrazole-1-carboxamide |
| 174 | 5-cyclobutyl-3-{2-hydroxy-4-[(2-methylbenzoyl)amino]phenyl}-N-propyl-1H- |
| | pyrazole-1-carboxamide |
| 175 | N-benzyl-5-cyclobutyl-3-{2-hydroxy-4-[(2-methylbenzoyl)amino]phenyl}-1H- |
| | pyrazole-1-carboxamide |
| 176 | 5-cyclobutyl-3-{2-hydroxy-4-[(2-methylbenzoyl)amino]phenyl}-N-(2- |
| | phenylethyl)-1H-pyrazole-1-carboxamide |
| 177 | 5-cyclobutyl-3-{2-hydroxy-4-[(2-methylbenzoyl)amino]phenyl}-N-(2- |
| | methylbenzyl)-1H-pyrazole-1-carboxamide |
| 178 | 5-cyclobutyl-3-{2-hydroxy-4-[(2-methylbenzoyl)amino]phenyl}-N-(3- |
| | methylbenzyl)-1H-pyrazole-1-carboxamide |
| 179 | 5-cyclobutyl-3-{2-hydroxy-4-[(2-methylbenzoyl)amino]phenyl}-N-(4- |
| | methylbenzyl)-1H-pyrazole-1-carboxamide |
| 180 | N-(2-chlorobenzyl)-5-cyclobutyl-3-{2-hydroxy-4-[(2- |
| | methylbenzoyl)amino]phenyl}-1H-pyrazole-1-carboxamide |
| 181 | 5-cyclobutyl-N-(4-fluorobenzyl)-3-{2-hydroxy-4-[(2- |
| | methylbenzoyl)amino]phenyl}-1H-pyrazole-1-carboxamide |
| 182 | 5-cyclobutyl-N-(3,4-dichlorobenzyl)-3-{2-hydroxy-4-[(2- |
| | methylbenzoyl)amino]phenyl}-1H-pyrazole-1-carboxamide |
| 183 | 5-cyclobutyl-3-{2-hydroxy-4-[(2-methylbenzoyl)amino]phenyl}-N-(4- |
| | methoxybenzyl)-1H-pyrazole-1-carboxamide |
| 184 | N-(sec-butyl)-5-cyclobutyl-3-{2-hydroxy-4-[(2-methylbenzoyl)amino]phenyl}- |
| | 1H-pyrazole-1-carboxamide |
| 185 | 5-cyclobutyl-N-(2-fluorobenzyl)-3-{2-hydroxy-4-[(2- |
| | methylbenzoyl)amino]phenyl}-1H-pyrazole-1-carboxamide |
| 186 | 5-cyclobutyl-N-(3-fluorobenzyl)-3-{2-hydroxy-4-[(2- |
| | methylbenzoyl)amino]phenyl}-1H-pyrazole-1-carboxamide |
| 187 | 5-cyclobutyl-N-cyclopropyl-3-{2-hydroxy-4-[(2-methylbenzoyl)amino]phenyl}- |
| | 1H-pyrazole-1-carboxamide |
| 188 | 5-cyclopentyl-3-{2-hydroxy-4-[(2-methylbenzoyl)amino]phenyl}-N-isopropyl- |
| | 1H-pyrazole-1-carboxamide |
| 189 | 5-cyclopentyl-N-ethyl-3-{2-hydroxy-4-[(2-methylbenzoyl)amino]phenyl}-1H- |
| | pyrazole-1-carboxamide |
| 190 | N-allyl-5-cyclopentyl-3-{2-hydroxy-4-[(2-methylbenzoyl)amino]phenyl}-1H- |
| | pyrazole-1-carboxamide |
| 191 | 5-cyclopentyl-3-{2-hydroxy-4-[(2-methylbenzoyl)amino]phenyl}-N-propyl-1H- |
| | pyrazole-1-carboxamide |
| 192 | N-benzyl-5-cyclopentyl-3-{2-hydroxy-4-[(2-methylbenzoyl)amino]phenyl}-1H- |
| | pyrazole-1-carboxamide |
| 193 | 5-cyclopentyl-3-{2-hydroxy-4-[(2-methylbenzoyl)amino]phenyl}-N-(2- |
| | phenylethyl)-1H-pyrazole-1-carboxamide |
| 194 | 5-cyclopentyl-3-{2-hydroxy-4-[(2-methylbenzoyl)amino]phenyl}-N-(2- |
| | methylbenzyl)-1H-pyrazole-1-carboxamide |
| 195 | 5-cyclopentyl-3-{2-hydroxy-4-[(2-methylbenzoyl)amino]phenyl}-N-(3- |
| | methylbenzyl)-1H-pyrazole-1-carboxamide |
| 196 | 5-cyclopentyl-3-{2-hydroxy-4-[(2-methylbenzoyl)amino]phenyl}-N-(4- |
| | methylbenzyl)-1H-pyrazole-1-carboxamide |
| 197 | N-(2-chlorobenzyl)-5-cyclopentyl-3-{2-hydroxy-4-[(2- |
| | methylbenzoyl)amino]phenyl}-1H-pyrazole-1-carboxamide |
| 198 | 5-cyclopentyl-N-(4-fluorobenzyl)-3-{2-hydroxy-4-[(2- |
| | methylbenzoyl)amino]phenyl}-1H-pyrazole-1-carboxamide |
| 199 | 5-cyclopentyl-N-(3,4-dichlorobenzyl)-3-{2-hydroxy-4-[(2- |
| | methylbenzoyl)amino]phenyl}-1H-pyrazole-1-carboxamide |
| 200 | 5-cyclopentyl-3-{2-hydroxy-4-[(2-methylbenzoyl)amino]phenyl}-N-(4- |
| | methoxybenzyl)-1H-pyrazole-1-carboxamide |
| 201 | N-(sec-butyl)-5-cyclopentyl-3-{2-hydroxy-4-[(2-methylbenzoyl)amino]phenyl}- |
| | 1H-pyrazole-1-carboxamide |
| 202 | 5-cyclopentyl-N-(2-fluorobenzyl)-3-{2-hydroxy-4-[(2- |
| | methylbenzoyl)amino]phenyl}-1H-pyrazole-1-carboxamide |
| 203 | 5-cyclopentyl-N-(3-fluorobenzyl)-3-{2-hydroxy-4-[(2- |
| | methylbenzoyl)amino]phenyl}-1H-pyrazole-1-carboxamide |
| 204 | 5-cyclopentyl-N-cyclopropyl-3-{2-hydroxy-4-[(2-methylbenzoyl)amino]phenyl}- |
| | 1H-pyrazole-1-carboxamide |
| 205 | 5-cyclopropyl-3-(2-hydroxy-4-{[3-(trifluoromethyl)benzoyl]amino}phenyl)-N- |
| | isopropyl-1H-pyrazole-1-carboxamide |
| 206 | 5-cyclopropyl-N-ethyl-3-(2-hydroxy-4-{[3- |
| | (trifluoromethyl)benzoyl]amino}phenyl)-1H-pyrazole-1-carboxamide |
| 207 | N-allyl-5-cyclopropyl-3-(2-hydroxy-4-{[3- |
| | (trifluoromethyl)benzoyl]amino}phenyl)-1H-pyrazole-1-carboxamide |
| 208 | 5-cyclopropyl-3-(2-hydroxy-4-{[3-(trifluoromethyl)benzoyl]amino}phenyl)-N- |
| | propyl-1H-pyrazole-1-carboxamide |
| 209 | N-benzyl-5-cyclopropyl-3-(2-hydroxy-4-{[3- |
| | (trifluoromethyl)benzoyl]amino}phenyl)-1H-pyrazole-1-carboxamide |
| 210 | 5-cyclopropyl-3-(2-hydroxy-4-{[3-(trifluoromethyl)benzoyl]amino}phenyl)-N-(2- |
| | phenylethyl)-1H-pyrazole-1-carboxamide |
| 211 | 5-cyclopropyl-3-(2-hydroxy-4-{[3-(trifluoromethyl)benzoyl]amino}phenyl)-N-(2- |
| | methylbenzyl)-1H-pyrazole-1-carboxamide |
| 212 | 5-cyclopropyl-3-(2-hydroxy-4-{[3-(trifluoromethyl)benzoyl]amino}phenyl)-N-(3- |
| | methylbenzyl)-1H-pyrazole-1-carboxamide |
| 213 | 5-cyclopropyl-3-(2-hydroxy-4-{[3-(trifluoromethyl)benzoyl]amino}phenyl)-N-(4- |
| | methylbenzyl)-1H-pyrazole-1-carboxamide |
| 214 | N-(2-chlorobenzyl)-5-cyclopropyl-3-(2-hydroxy-4-{[3- |
| | (trifluoromethyl)benzoyl]amino}phenyl)-1H-pyrazole-1-carboxamide |
| 215 | 5-cyclopropyl-N-(4-fluorobenzyl)-3-(2-hydroxy-4-{[3- |
| | (trifluoromethyl)benzoyl]amino}phenyl)-1H-pyrazole-1-carboxamide |
| 216 | 5-cyclopropyl-N-(3,4-dichlorobenzyl)-3-(2-hydroxy-4-{[3- |
| | (trifluoromethyl)benzoyl]amino}phenyl)-1H-pyrazole-1-carboxamide |
| 217 | 5-cyclopropyl-3-(2-hydroxy-4-{[3-(trifluoromethyl)benzoyl]amino}phenyl)-N-(4- |
| | methoxybenzyl)-1H-pyrazole-1-carboxamide |
| 218 | N-(sec-butyl)-5-cyclopropyl-3-(2-hydroxy-4-{[3- |
| | (trifluoromethyl)benzoyl]amino}phenyl)-1H-pyrazole-1-carboxamide |
| 219 | 5-cyclopropyl-N-(2-fluorobenzyl)-3-(2-hydroxy-4-{[3- |
| | (trifluoromethyl)benzoyl]amino}phenyl)-1H-pyrazole-1-carboxamide |
| 220 | N-allyl-5-cyclobutyl-3-(2-hydroxy-4-{[3- |
| | (trifluoromethyl)benzoyl]amino}phenyl)-1H-pyrazole-1-carboxamide |
| 221 | N,5-dicyclopropyl-3-(2-hydroxy-4-{[3-(trifluoromethyl)benzoyl]amino}phenyl)- |
| | 1H-pyrazole-1-carboxamide |
| 222 | 5-cyclobutyl-3-(2-hydroxy-4-{[3-(trifluoromethyl)benzoyl]amino}phenyl)-N- |
| | isopropyl-1H-pyrazole-1-carboxamide |
| 223 | 5-cyclobutyl-N-ethyl-3-(2-hydroxy-4-{[3- |
| | (trifluoromethyl)benzoyl]amino}phenyl)-1H-pyrazole-1-carboxamide |
| 224 | N-allyl-5-cyclobutyl-3-(2-hydroxy-4-{[3- |
| | (trifluoromethyl)benzoyl]amino}phenyl)-1H-pyrazole-1-carboxamide |
| 225 | 5-cyclobutyl-3-(2-hydroxy-4-{[3-(trifluoromethyl)benzoyl]amino}phenyl)-N- |
| | propyl-1H-pyrazole-1-carboxamide |
| 226 | N-benzyl-5-cyclobutyl-3-(2-hydroxy-4-{[3- |
| | (trifluoromethyl)benzoyl]amino}phenyl)-1H-pyrazole-1-carboxamide |
| 227 | 5-cyclobutyl-3-(2-hydroxy-4-{[3-(trifluoromethyl)benzoyl]amino}phenyl)-N-(2- |
| | phenylethyl)-1H-pyrazole-1-carboxamide |
| 228 | 5-cyclobutyl-3-(2-hydroxy-4-{[3-(trifluoromethyl)benzoyl]amino}phenyl)-N-(2- |
| | methylbenzyl)-1H-pyrazole-1-carboxamide |
| 229 | 5-cyclobutyl-3-(2-hydroxy-4-{[3-(trifluoromethyl)benzoyl]amino}phenyl)-N-(3- |
| | methylbenzyl)-1H-pyrazole-1-carboxamide |
| 230 | 5-cyclobutyl-3-(2-hydroxy-4-{[3-(trifluoromethyl)benzoyl]amino}phenyl)-N-(4- |
| | methylbenzyl)-1H-pyrazole-1-carboxamide |
| 231 | N-(2-chlorobenzyl)-5-cyclobutyl-3-(2-hydroxy-4-{[3- |
| | (trifluoromethyl)benzoyl]amino}phenyl)-1H-pyrazole-1-carboxamide |
| 232 | 5-cyclobutyl-N-(4-fluorobenzyl)-3-(2-hydroxy-4-{[3- |
| | (trifluoromethyl)benzoyl]amino}phenyl)-1H-pyrazole-1-carboxamide |
| 233 | 5-cyclobutyl-N-(3 ,4-dichlorobenzyl)-3-(2-hydroxy-4-{[3- |
| | (trifluoromethyl)benzoyl]amino}phenyl)-1H-pyrazole-1-carboxamide |
| 234 | 5-cyclobutyl-3-(2-hydroxy-4-{[3-(trifluoromethyl)benzoyl]amino}phenyl)-N-(4- |
| | methoxybenzyl)-1H-pyrazole-1-carboxamide |
| 235 | N-(sec-butyl)-5-cyclobutyl-3-(2-hydroxy-4-{[3- |
| | (trifluoromethyl)benzoyl]amino}phenyl)-1H-pyrazole-1-carboxamide |
| 236 | 5-cyclobutyl-N-(2-fluorobenzyl)-3-(2-hydroxy-4-{[3- |
| | (trifluoromethyl)benzoyl]amino}phenyl)-1H-pyrazole-1-carboxamide |
| 237 | 5-cyclobutyl-N-(3-fluorobenzyl)-3-(2-hydroxy-4-{[3- |
| | (trifluoromethyl)benzoyl]amino}phenyl)-1H-pyrazole-1-carboxamide |
| 238 | 5-cyclobutyl-N-cyclopropyl-3-(2-hydroxy-4-{[3- |
| | (trifluoromethyl)benzoyl]amino}phenyl)-1H-pyrazole-1-carboxamide |
| 239 | 5-cyclopentyl-3-(2-hydroxy-4-{[3-(trifluoromethyl)benzoyl]amino}phenyl)-N- |
| | isopropyl-1H-pyrazole-1-carboxamide |
| 240 | 5-cyclopentyl-N-ethyl-3-(2-hydroxy-4-{[3- |
| | (trifluoromethyl)benzoyl]amino}phenyl)-1H-pyrazole-1-carboxamide |
| 241 | N-allyl-5-cyclopentyl-3-(2-hydroxy-4-{[3- |
| | (trifluoromethyl)benzoyl]amino}phenyl)-1H-pyrazole-1-carboxamide |
| 242 | 5-cyclopentyl-3-(2-hydroxy-4-{[3-(trifluoromethyl)benzoyl]amino}phenyl)-N- |
| | propyl-1H-pyrazole-1-carboxamide |
| 243 | N-benzyl-5-cyclopentyl-3-(2-hydroxy-4-{[3- |
| | (trifluoromethyl)benzoyl]amino}phenyl)-1H-pyrazole-1-carboxamide |
| 244 | 5-cyclopentyl-3-(2-hydroxy-4-{[3-(trifluoromethyl)benzoyl]amino}phenyl)-N-(2- |
| | phenylethyl)-1H-pyrazole-1-carboxamide |
| 245 | 5-cyclopentyl-3-(2-hydroxy-4-{[3-(trifluoromethyl)benzoyl]amino}phenyl)-N-(2- |
| | methylbenzyl)-1H-pyrazole-1-carboxamide |
| 246 | 5-cyclopentyl-3-(2-hydroxy-4-{[3-(trifluoromethyl)benzoyl]amino}phenyl)-N-(3- |
| | methylbenzyl)-1H-pyrazole-1-carboxamide |
| 247 | 5-cyclopentyl-3-(2-hydroxy-4-{[3-(trifluoromethyl)benzoyl]amino}phenyl)-N-(4- |
| | methylbenzyl)-1H-pyrazole-1-carboxamide |
| 248 | N-(2-chlorobenzyl)-5-cyclopentyl-3-(2-hydroxy-4-{[3- |
| | (trifluoromethyl)benzoyl]amino}phenyl)-1H-pyrazole-1-carboxamide |
| 249 | 5-cyclopentyl-N-(4-fluorobenzyl)-3-(2-hydroxy-4-{[3- |
| | (trifluoromethyl)benzoyl]amino}phenyl)-1H-pyrazole-1-carboxamide |
| 250 | 5-cyclopentyl-N-(3,4-dichlorobenzyl)-3-(2-hydroxy-4-{[3- |
| | (trifluoromethyl)benzoyl]amino}phenyl)-1H-pyrazole-1-carboxamide |
| 251 | 5-cyclopentyl-3-(2-hydroxy-4-{[3-(trifluoromethyl)benzoyl]amino}phenyl)-N-(4- |
| | methoxybenzyl)-1H-pyrazole-1-carboxamide |
| 252 | N-(sec-butyl)-5-cyclopentyl-3-(2-hydroxy-4-{[3- |
| | (trifluoromethyl)benzoyl]amino}phenyl)-1H-pyrazole-1-carboxamide |
| 253 | 5-cyclopentyl-N-(2-fluorobenzyl)-3-(2-hydroxy-4-{[3- |
| | (trifluoromethyl)benzoyl]amino}phenyl)-1H-pyrazole-1-carboxamide |
| 254 | 5-cyclopentyl-N-(3-fluorobenzyl)-3-(2-hydroxy-4-{[3- |
| | (trifluoromethyl)benzoyl]amino}phenyl)-1H-pyrazole-1-carboxamide |
| 255 | 5-cyclopentyl-N-cyclopropyl-3-(2-hydroxy-4-{[3- |
| | (trifluoromethyl)benzoyl]amino}phenyl)-1H-pyrazole-1-carboxamide |
| 256 | 3-{4-[(4-chlorobenzoyl)amino]-2-hydroxyphenyl}-5-cyclopropyl-N-isopropyl- |
| | 1H-pyrazole-1-carboxamide |
| 257 | 3-{4-[(4-chlorobenzoyl)amino]-2-hydroxyphenyl}-5-cyclopropyl-N-ethyl-1H- |
| | pyrazole-1-carboxamide |
| 258 | N-allyl-3-{4-[(4-chlorobenzoyl)amino]-2-hydroxyphenyl}-5-cyclopropyl-1H- |
| | pyrazole-1-carboxamide |
| 259 | 3-{4-[(4-chlorobenzoyl)amino]-2-hydroxyphenyl}-5-cyclopropyl-N-propyl-1H- |
| | pyrazole-1-carboxamide |
| 260 | N-benzyl-3-{4-[(4-chlorobenzoyl)amino]-2-hydroxyphenyl}-5-cyclopropyl-1H- |
| | pyrazole-1-carboxamide |
| 261 | 3-{4-[(4-chlorobenzoyl)amino]-2-hydroxyphenyl}-5-cyclopropyl-N-(2- |
| | phenylethyl)-1H-pyrazole-1-carboxamide |
| 262 | 3-{4-[(4-chlorobenzoyl)amino]-2-hydroxyphenyl}-5-cyclopropyl-N-(4- |
| | fluorobenzyl)-1H-pyrazole-1-carboxamide |
| 263 | 3-{4-[(4-chlorobenzoyl)amino]-2-hydroxyphenyl}-5-cyclopropyl-N-(3- |
| | methylbenzyl)-1H-pyrazole-1-carboxamide |
| 264 | 3-{4-[(4-chlorobenzoyl)amino]-2-hydroxyphenyl}-5-cyclopropyl-N-(4- |
| | methylbenzyl)-1H-pyrazole-1-carboxamide |
| 265 | 3-{4-[(4-chlorobenzoyl)amino]-2-hydroxyphenyl}-N-(2-chlorobenzyl)-5- |
| | cyclopropyl-1H-pyrazole-1-carboxamide |
| 266 | 3-{4-[(4-chlorobenzoyl)amino]-2-hydroxyphenyl}-5-cyclopropyl-N-(4- |
| | fluorobenzyl)-1H-pyrazole-1-carboxamide |
| 267 | 3-{4-[(4-chlorobenzoyl)amino]-2-hydroxyphenyl}-5-cyclopropyl-N-(3,4- |
| | dichlorobenzyl)-1H-pyrazole-1-carboxamide |
| 268 | 3-{4-[(4-chlorobenzoyl)amino]-2-hydroxyphenyl}-5-cyclopropyl-N-(4- |
| | methoxybenzyl)-1H-pyrazole-1-carboxamide |
| 269 | N-(sec-butyl)-3-{4-[(4-chlorobenzoyl)amino]-2-hydroxyphenyl}-5-cyclopropyl- |
| | 1H-pyrazole-1-carboxamide |
| 270 | 3-{4-[(4-chlorobenzoyl)amino]-2-hydroxyphenyl}-5-cyclopropyl-N-(2- |
| | fluorobenzyl)-1H-pyrazole-1-carboxamide |
| 271 | 3-{4-[(4-chlorobenzoyl)amino]-2-hydroxyphenyl}-5-cyclopropyl-N-(3- |
| | fluorobenzyl)-1H-pyrazole-1-carboxamide |
| 272 | 3-{4-[(4-chlorobenzoyl)amino]-2-hydroxyphenyl}-N,5-dicyclopropyl-1H- |
| | pyrazole-1-carboxamide |
| 273 | 3-{4-[(4-chlorobenzoyl)amino]-2-hydroxyphenyl}-5-cyclobutyl-N-isopropyl-1H- |
| | pyrazole-1-carboxamide |
| 274 | 3-{4-[(4-chlorobenzoyl)amino]-2-hydroxyphenyl}-5-cyclobutyl-N-ethyl-1H- |
| | pyrazole-1-carboxamide |
| 275 | N-allyl-3-{4-[(4-chlorobenzoyl)amino]-2-hydroxyphenyl}-5-cyclobutyl-1H- |
| | pyrazole-1-carboxamide |
| 276 | 3-{4-[(4-chlorobenzoyl)amino]-2-hydroxyphenyl}-5-cyclobutyl-N-propyl-1H- |
| | pyrazole-1-carboxamide |
| 277 | N-benzyl-3-{4-[(4-chlorobenzoyl)amino]-2-hydroxyphenyl}-5-cyclobutyl-1H- |
| | pyrazole-1-carboxamide |
| 278 | 3-{4-[(4-chlorobenzoyl)amino]-2-hydroxyphenyl}-5-cyclobutyl-N-(2- |
| | phenylethyl)-1H-pyrazole-1-carboxamide |
| 279 | 3-{4-[(4-chlorobenzoyl)amino]-2-hydroxyphenyl}-5-cyclobutyl-N-(2- |
| | methylbenzyl)-1H-pyrazole-1-carboxamide |
| 280 | 3-{4-[(4-chlorobenzoyl)amino]-2-hydroxyphenyl}-5-cyclobutyl-N-(3- |
| | methylbenzyl)-1H-pyrazole-1-carboxamide |
| 281 | 3-{4-[(4-chlorobenzoyl)amino]-2-hydroxyphenyl}-5-cyclobutyl-N-(4- |
| | methylbenzyl)-1H-pyrazole-1-carboxamide |
| 282 | 3-{4-[(4-chlorobenzoyl)amino]-2-hydroxyphenyl}-N-(2-chlorobenzyl)-5- |
| | cyclobutyl-1H-pyrazole-1-carboxamide |
| 283 | 3-{4-[(4-chlorobenzoyl)amino]-2-hydroxyphenyl}-5-cyclobutyl-N-(4- |
| | fluorobenzyl)-1H-pyrazole-1-carboxamide |
| 284 | 3-{4-[(4-chlorobenzoyl)amino]-2-hydroxyphenyl}-5-cyclobutyl-N-(3,4- |
| | dichlorobenzyl)-1H-pyrazole-1-carboxamide |
| 285 | 3-{4-[(4-chlorobenzoyl)amino]-2-hydroxyphenyl}-5-cyclobutyl-N-(4- |
| | methoxybenzyl)-1H-pyrazole-1-carboxamide |
| 286 | N-(sec-butyl)-3-{4-[(4-chlorobenzoyl)amino]-2-hydroxyphenyl}-5-cyclobutyl- |
| | 1H-pyrazole-1-carboxamide |
| 287 | 3-{4-[(4-chlorobenzoyl)amino]-2-hydroxyphenyl}-5-cyclobutyl-N-(2- |
| | fluorobenzyl)-1H-pyrazole-1-carboxamide |
| 288 | 3-{4-[(4-chlorobenzoyl)amino]-2-hydroxyphenyl}-5-cyclobutyl-N-(3- |
| | fluorobenzyl)-1H-pyrazole-1-carboxamide |
| 289 | 3-{4-[(4-chlorobenzoyl)amino]-2-hydroxyphenyl}-5-cyclobutyl-N-cyclopropyl- |
| | 1H-pyrazole-1-carboxamide |
| 290 | 3-{4-[(4-chlorobenzoyl)amino]-2-hydroxyphenyl}-5-cyclopentyl-N-isopropyl- |
| | 1H-pyrazole-1-carboxamide |
| 291 | 3-{4-[(4-chlorobenzoyl)amino]-2-hydroxyphenyl}-5-cyclopentyl-N-ethyl-1H- |
| | pyrazole-1-carboxamide |
| 292 | N-allyl-3-{4-[(4-chlorobenzoyl)amino]-2-hydroxyphenyl}-5-cyclopentyl-1H- |
| | pyrazole-1-carboxamide |
| 293 | 3-{4-[(4-chlorobenzoyl)amino]-2-hydroxyphenyl}-5-cyclopentyl-N-propyl-1H- |
| | pyrazole-1-carboxamide |
| 294 | N-benzyl-3-{4-[(4-chlorobenzoyl)amino]-2-hydroxyphenyl}-5-cyclopentyl-1H- |
| | pyrazole-1-carboxamide |
| 295 | 3-{4-[(4-chlorobenzoyl)amino]-2-hydroxyphenyl}-5-cyclopentyl-N-(2- |
| | phenylethyl)-1H-pyrazole-1-carboxamide |
| 296 | 3-{4-[(4-chlorobenzoyl)amino]-2-hydroxyphenyl}-5-cyclopentyl-N-(2- |
| | methylbenzyl)-1H-pyrazole-1-carboxamide |
| 297 | 3-{4-[(4-chlorobenzoyl)amino]-2-hydroxyphenyl}-5-cyclopentyl-N-(3- |
| | methylbenzyl)-1H-pyrazole-1-carboxamide |
| 298 | 3-{4-[(4-chlorobenzoyl)amino]-2-hydroxyphenyl}-5-cyclopentyl-N-(4- |
| | methylbenzyl)-1H-pyrazole-1-carboxamide |
| 299 | 3-{4-[(4-chlorobenzoyl)amino]-2-hydroxyphenyl}-N-(2-chlorobenzyl)-5- |
| | cyclopentyl-1H-pyrazole-1-carboxamide |
| 300 | 3-{4-[(4-chlorobenzoyl)amino]-2-hydroxyphenyl}-5-cyclopentyl-N-(4- |
| | fluorobenzyl)-1H-pyrazole-1-carboxamide |
| 301 | 3-{4-[(4-chlorobenzoyl)amino]-2-hydroxyphenyl}-5-cyclopentyl-N-(3,4- |
| | dichlorobenzyl)-1H-pyrazole-1-carboxamide |
| 302 | 3-{4-[(4-chlorobenzoyl)amino]-2-hydroxyphenyl}-5-cyclopentyl-N-(4- |
| | methoxybenzyl)-1H-pyrazole-1-carboxamide |
| 303 | N-(sec-butyl)-3-{4-[(4-chlorobenzoyl)amino]-2-hydroxyphenyl}-5-cyclopentyl- |
| | 1H-pyrazole-1-carboxamide |
| 304 | 3-{4-[(4-chlorobenzoyl)amino]-2-hydroxyphenyl}-5-cyclopentyl-N-(2- |
| | fluorobenzyl)-1H-pyrazole-1-carboxamide |
| 305 | 3-{4-[(4-chlorobenzoyl)amino]-2-hydroxyphenyl}-5-cyclopentyl-N-(3- |
| | fluorobenzyl)-1H-pyrazole-1-carboxamide |
| 306 | 3-{4-[(4-chlorobenzoyl)amino]-2-hydroxyphenyl}-5-cyclopentyl-N-cyclopropyl- |
| | 1H-pyrazole-1-carboxamide |
| 307 | 5-cyclopropyl-3-{4-[(4-ethoxybenzoyl)amino]-2-hydroxyphenyl}-N-isopropyl- |
| | 1H-pyrazole-1-carboxamide |
| 308 | 5-cyclopropyl-3-{4-[(4-ethoxybenzoyl)amino]-2-hydroxyphenyl}-N-ethyl-1H- |
| | pyrazole-1-carboxamide |
| 309 | N-allyl-5-cyclopropyl-3-{4-[(4-ethoxybenzoyl)amino]-2-hydroxyphenyl}-1H- |
| | pyrazole-1-carboxamide |
| 310 | 5-cyclopropyl-3-{4-[(4-ethoxybenzoyl)amino]-2-hydroxyphenyl}-N-propyl-1H- |
| | pyrazole-1-carboxamide |
| 311 | N-benzyl-5-cyclopropyl-3-{4-[(4-ethoxybenzoyl)amino]-2-hydroxyphenyl}-1H- |
| | pyrazole-1-carboxamide |
| 312 | 5-cyclopropyl-3-{4-[(4-ethoxybenzoyl)amino]-2-hydroxyphenyl}-N-(2- |
| | phenylethyl)-1H-pyrazole-1-carboxamide |
| 313 | 5-cyclopropyl-3-{4-[(4-ethoxybenzoyl)amino]-2-hydroxyphenyl}-N-(2- |
| | methylbenzyl)-1H-pyrazole-1-carboxamide |
| 314 | 5-cyclopropyl-3-{4-[(4-ethoxybenzoyl)amino]-2-hydroxyphenyl}-N-(3- |
| | methylbenzyl)-1H-pyrazole-1-carboxamide |
| 315 | 5-cyclopropyl-3-{4-[(4-ethoxybenzoyl)amino]-2-hydroxyphenyl}-N-(4- |
| | methylbenzyl)-1H-pyrazole-1-carboxamide |
| 316 | N-(2-chlorobenzyl)-5-cyclopropyl-3-{4-[(4-ethoxybenzoyl)amino]-2- |
| | hydroxyphenyl}-1H-pyrazole-1-carboxamide |
| 317 | 5-cyclopropyl-3-{4-[(4-ethoxybenzoyl)amino]-2-hydroxyphenyl}-N-(4- |
| | fluorobenzyl)-1H-pyrazole-1-carboxamide |
| 318 | 5-cyclopropyl-N-(3,4-dichlorobenzyl)-3-{4-[(4-ethoxybenzoyl)amino]-2- |
| | hydroxyphenyl}-1H-pyrazole-1-carboxamide |
| 319 | 5-cyclopropyl-3-{4-[(4-ethoxybenzoyl)amino]-2-hydroxyphenyl}-N-(4- |
| | methoxybenzyl)-1H-pyrazole-1-carboxamide |
| 320 | N-(sec-butyl)-5-cyclopropyl-3-{4-[(4-ethoxybenzoyl)amino]-2-hydroxyphenyl}- |
| | 1H-pyrazole-1-carboxamide |
| 321 | 5-cyclopropyl-3-{4-[(4-ethoxybenzoyl)amino]-2-hydroxyphenyl}-N-(2- |
| | fluorobenzyl)-1H-pyrazole-1-carboxamide |
| 322 | 5-cyclopropyl-3-{4-[(4-ethoxybenzoyl)amino]-2-hydroxyphenyl}-N-(3- |
| | fluorobenzyl)-1H-pyrazole-1-carboxamide |
| 323 | N,5-dicyclopropyl-3-{4-[(4-ethoxybenzoyl)amino]-2-hydroxyphenyl}-1H- |
| | pyrazole-1-carboxamide |
| 324 | 5-cyclobutyl-3-{4-[(4-ethoxybenzoyl)amino]-2-hydroxyphenyl}-N-isopropyl-1H- |
| | pyrazole-1-carboxamide |
| 325 | 5-cyclobutyl-3-{4-[(4-ethoxybenzoyl)amino]-2-hydroxyphenyl}-N-ethyl-1H- |
| | pyrazole-1-carboxamide |
| 326 | N-allyl-5-cyclobutyl-3-{4-[(4-ethoxybenzoyl)amino]-2-hydroxyphenyl}-1H- |
| | pyrazole-1-carboxamide |
| 327 | 5-cyclobutyl-3-{4-[(4-ethoxybenzoyl)amino]-2-hydroxyphenyl}-N-propyl-1H- |
| | pyrazole-1-carboxamide |
| 328 | N-benzyl-5-cyclobutyl-3-{4-[(4-ethoxybenzoyl)amino]-2-hydroxyphenyl}-1H- |
| | pyrazole-1-carboxamide |
| 329 | 5-cyclobutyl-3-{4-[(4-ethoxybenzoyl)amino]-2-hydroxyphenyl}-N-(2- |
| | phenylethyl)-1H-pyrazole-1-carboxamide |
| 330 | 5-cyclobutyl-3-{4-[(4-ethoxybenzoyl)amino]-2-hydroxyphenyl}-N-(2- |
| | methylbenzyl)-1H-pyrazole-1-carboxamide |
| 331 | 5-cyclobutyl-3-{4-[(4-ethoxybenzoyl)amino]-2-hydroxyphenyl}-N-(3- |
| | methylbenzyl)-1H-pyrazole-1-carboxamide |
| 332 | 5-cyclobutyl-3-{4-[(4-ethoxybenzoyl)amino]-2-hydroxyphenyl}-N-(4- |
| | methylbenzyl)-1H-pyrazole-1-carboxamide |
| 333 | N-(2-chlorobenzyl)-5-cyclobutyl-3-{4-[(4-ethoxybenzoyl)amino]-2- |
| | hydroxyphenyl}-1H-pyrazole-1-carboxamide |
| 334 | 5-cyclobutyl-3-{4-[(4-ethoxybenzoyl)amino]-2-hydroxyphenyl}-N-(4- |
| | fluorobenzyl)-1H-pyrazole-1-carboxamide |
| 335 | 5-cyclobutyl-N-(3,4-dichlorobenzyl)-3-{4-[(4-ethoxybenzoyl)amino]-2- |
| | hydroxyphenyl}-1H-pyrazole-1-carboxamide |
| 336 | 5-cyclobutyl-3-{4-[(4-ethoxybenzoyl)amino]-2-hydroxyphenyl}-N-(4- |
| | methoxybenzyl)-1H-pyrazole-1-carboxamide |
| 337 | N-(sec-butyl)-5-cyclobutyl-3-{4-[(4-ethoxybenzoyl)amino]-2-hydroxyphenyl}- |
| | 1H-pyrazole-1-carboxamide |
| 338 | 5-cyclobutyl-3-{4-[(4-ethoxybenzoyl)amino]-2-hydroxyphenyl}-N-(2- |
| | fluorobenzyl)-1H-pyrazole-1-carboxamide |
| 339 | 5-cyclobutyl-3-{4-[(4-ethoxybenzoyl)amino]-2-hydroxyphenyl}-N-(3- |
| | fluorobenzyl)-1H-pyrazole-1-carboxamide |
| 340 | 5-cyclobutyl-N-cyclopropyl-3-{4-[(4-ethoxybenzoyl)amino]-2-hydroxyphenyl}- |
| | 1H-pyrazole-1-carboxamide |
| 341 | 5-cyclopentyl-3-{4-[(4-ethoxybenzoyl)amino]-2-hydroxyphenyl}-N-isopropyl- |
| | 1H-pyrazole-1-carboxamide |
| 342 | 5-cyclopentyl-3-{4-[(4-ethoxybenzoyl)amino]-2-hydroxyphenyl}-N-ethyl-1H- |
| | pyrazole-1-carboxamide |
| 343 | N-allyl-5-cyclopentyl-3-{4-[(4-ethoxybenzoyl)amino]-2-hydroxyphenyl}-1H- |
| | pyrazole-1-carboxamide |
| 344 | 5-cyclopentyl-3-{4-[(4-ethoxybenzoyl)amino]-2-hydroxyphenyl}-N-propyl-1H- |
| | pyrazole-1-carboxamide |
| 345 | N-benzyl-5-cyclopentyl-3-{4-[(4-ethoxybenzoyl)amino]-2-hydroxyphenyl}-1H- |
| | pyrazole-1-carboxamide |
| 346 | 5-cyclopentyl-3-{4-[(4-ethoxybenzoyl)amino]-2-hydroxyphenyl}-N-(2- |
| | phenylethyl)-1H-pyrazole-1-carboxamide |
| 347 | 5-cyclopentyl-3-{4-[(4-ethoxybenzoyl)amino]-2-hydroxyphenyl}-N-(2- |
| | methylbenzyl)-1H-pyrazole-1-carboxamide |
| 348 | 5-cyclopentyl-3-{4-[(4-ethoxybenzoyl)amino]-2-hydroxyphenyl}-N-(3- |
| | methylbenzyl)-1H-pyrazole-1-carboxamide |
| 349 | 5-cyclopentyl-3-{4-[(4-ethoxybenzoyl)amino]-2-hydroxyphenyl}-N-(4- |
| | methylbenzyl)-1H-pyrazole-1-carboxamide |
| 350 | N-(2-chlorobenzyl)-5-cyclopentyl-3-{4-[(4-ethoxybenzoyl)amino]-2- |
| | hydroxyphenyl}-1H-pyrazole-1-carboxamide |
| 351 | 5-cyclopentyl-3-{4-[(4-ethoxybenzoyl)amino]-2-hydroxyphenyl}-N-(4- |
| | fluorobenzyl)-1H-pyrazole-1-carboxamide |
| 352 | 5-cyclopentyl-N-(3,4-dichlorobenzyl)-3-{4-[(4-ethoxybenzoyl)amino]-2- |
| | hydroxyphenyl}-1H-pyrazole-1-carboxamide |
| 353 | 5-cyclopentyl-3-{4-[(4-ethoxybenzoyl)amino]-2-hydroxyphenyl}-N-(4- |
| | methoxybenzyl)-1H-pyrazole-1-carboxamide |
| 354 | N-(sec-butyl)-5-cyclopentyl-3-{4-[(4-ethoxybenzoyl)amino]-2-hydroxyphenyl}- |
| | 1H-pyrazole-1-carboxamide |
| 355 | 5-cyclopentyl-3-{4-[(4-ethoxybenzoyl)amino]-2-hydroxyphenyl}-N-(2- |
| | fluorobenzyl)-1H-pyrazole-1-carboxamide |
| 356 | 5-cyclopentyl-3-{4-[(4-ethoxybenzoyl)amino]-2-hydroxyphenyl}-N-(3- |
| | fluorobenzyl)-1H-pyrazole-1-carboxamide |
| 357 | 5-cyclopentyl-N-cyclopropyl-3-{4-[(4-ethoxybenzoyl)amino]-2-hydroxyphenyl}- |
| | 1H-pyrazole-1-carboxamide |
| 358 | 3-{4-[(4-tert-butylbenzoyl)amino]-2-hydroxyphenyl}-5-cyclopropyl-N-propyl- |
| | 1H-pyrazole-1-carboxamide |
| 359 | 3-{4-[(4-tert-butylbenzoyl)amino]-2-hydroxyphenyl}-5-cyclopropyl-N-ethyl-1H- |
| | pyrazole-1-carboxamide |
| 360 | N-allyl-3-{4-[(4-tert-butylbenzoyl)amino]-2-hydroxyphenyl}-5-cyclopropyl-1H- |
| | pyrazole-1-carboxamide |
| 361 | 3-{4-[(4-tert-butylbenzoyl)amino]-2-hydroxyphenyl}-5-cyclopropyl-N-propyl- |
| | 1H-pyrazole-1-carboxamide |
| 362 | N-benzyl-3-{4-[(4-tert-butylbenzoyl)amino]-2-hydroxyphenyl}-5-cyclopropyl- |
| | 1H-pyrazole-1-carboxamide |
| 363 | 3-{4-[(4-tert-butylbenzoyl)amino]-2-hydroxyphenyl}-5-cyclopropyl-N-(2- |
| | phenylethyl)-1H-pyrazole-1-carboxamide |
| 364 | 3-{4-[(4-tert-butylbenzoyl)amino]-2-hydroxyphenyl}-5-cyclopropyl-N-(2- |
| | methylbenzyl)-1H-pyrazole-1-carboxamide |
| 365 | 3-{4-[(4-tert-butylbenzoyl)amino]-2-hydroxyphenyl}-5-cyclopropyl-N-(3- |
| | methylbenzyl)-1H-pyrazole-1-carboxamide |
| 366 | 3-{4-[(4-tert-butylbenzoyl)amino]-2-hydroxyphenyl}-5-cyclopropyl-N-(4- |
| | methylbenzyl)-1H-pyrazole-1-carboxamide |
| 367 | 3-{4-[(4-tert-butylbenzoyl)amino]-2-hydroxyphenyl}-N-(2-chlorobenzyl)-5- |
| | cyclopropyl-1H-pyrazole-1-carboxamide |
| 368 | 3-{4-[(4-tert-butylbenzoyl)amino]-2-hydroxyphenyl}-5-cyclopropyl-N-(4- |
| | fluorobenzyl)-1H-pyrazole-1-carboxamide |
| 369 | 3-{4-[(4-tert-butylbenzoyl)amino]-2-hydroxyphenyl}-5-cyclopropyl-N-(3,4- |
| | dichlorobenzyl)-1H-pyrazole-1-carboxamide |
| 370 | 3-{4-[(4-tert-butylbenzoyl)amino]-2-hydroxyphenyl}-5-cyclopropyl-N-(4- |
| | methoxybenzyl)-1H-pyrazole-1-carboxamide |
| 371 | N-(sec-butyl)-3-{4-[(4-tert-butylbenzoyl)amino]-2-hydroxyphenyl}-5- |
| | cyclopropyl-1H-pyrazole-1-carboxamide |
| 372 | 3-{4-[(4-tert-butylbenzoyl)amino]-2-hydroxyphenyl}-5-cyclopropyl-N-(2- |
| | fluorobenzyl)-1H-pyrazole-1-carboxamide |
| 373 | 3-{4-[(4-tert-butylbenzoyl)amino]-2-hydroxyphenyl}-5-cyclopropyl-N-(3- |
| | fluorobenzyl)-1H-pyrazole-1-carboxamide |
| 374 | 3-{4-[(4-tert-butylbenzoyl)amino]-2-hydroxyphenyl}-N,5-dicyclopropyl-1H- |
| | pyrazole-1-carboxamide |
| 375 | 3-{4-[(4-tert-butylbenzoyl)amino]-2-hydroxyphenyl}-5-cyclobutyl-N-isopropyl- |
| | 1H-pyrazole-1-carboxamide |
| 376 | 3-{4-[(4-tert-butylbenzoyl)amino]-2-hydroxyphenyl}-5-cyclobutyl-N-ethyl-1H- |
| | pyrazole-1-carboxamide |
| 377 | N-allyl-3-{4-[(4-tert-butylbenzoyl)amino]-2-hydroxyphenyl}-5-cyclobutyl-1H- |
| | pyrazole-1-carboxamide |
| 378 | 3-{4-[(4-tert-butylbenzoyl)amino]-2-hydroxyphenyl}-5-cyclobutyl-N-propyl-1H- |
| | pyrazole-1-carboxamide |
| 379 | N-benzyl-3-{4-[(4-tert-butylbenzoyl)amino]-2-hydroxyphenyl}-5-cyclobutyl-1H- |
| | pyrazole-1-carboxamide |
| 380 | 3-{4-[(4-tert-butylbenzoyl)amino]-2-hydroxyphenyl}-5-cyclobutyl-N-(2- |
| | phenylethyl)-1H-pyrazole-1-carboxamide |
| 381 | 3-{4-[(4-tert-butylbenzoyl)amino]-2-hydroxyphenyl}-5-cyclobutyl-N-(2- |
| | methylbenzyl)-1H-pyrazole-1-carboxamide |
| 382 | 3-{4-[(4-tert-butylbenzoyl)amino]-2-hydroxyphenyl}-5-cyclobutyl-N-(3- |
| | methylbenzyl)-1H-pyrazole-1-carboxamide |
| 383 | 3-{4-[(4-tert-butylbenzoyl)amino]-2-hydroxyphenyl}-5-cyclobutyl-N-(4- |
| | methylbenzyl)-1H-pyrazole-1-carboxamide |
| 384 | 3-{4-[(4-tert-butylbenzoyl)amino]-2-hydroxyphenyl}-N-(2-chlorobenzyl)-5- |
| | cyclobutyl-1H-pyrazole-1-carboxamide |
| 385 | 3-{4-[(4-tert-butylbenzoyl)amino]-2-hydroxyphenyl}-5-cyclobutyl-N-(4- |
| | fluorobenzyl)-1H-pyrazole-1-carboxamide |
| 386 | 3-{4-[(4-tert-butylbenzoyl)amino]-2-hydroxyphenyl}-5-cyclobutyl-N-(3,4- |
| | dichlorobenzyl)-1H-pyrazole-1-carboxamide |
| 387 | 3-{4-[(4-tert-butylbenzoyl)amino]-2-hydroxyphenyl}-5-cyclobutyl-N-(4- |
| | methoxybenzyl)-1H-pyrazole-1-carboxamide |
| 388 | N-(sec-butyl)-3-{4-[(4-tert-butylbenzoyl)amino]-2-hydroxyphenyl}-5-cyclobutyl- |
| | 1H-pyrazole-1-carboxamide |
| 389 | 3-{4-[(4-tert-butylbenzoyl)amino]-2-hydroxyphenyl}-5-cyclobutyl-N-(2- |
| | fluorobenzyl)-1H-pyrazole-1-carboxamide |
| 390 | 3-{4-[(4-tert-butylbenzoyl)amino]-2-hydroxyphenyl}-5-cyclobutyl-N-(3- |
| | fluorobenzyl)-1H-pyrazole-1-carboxamide |
| 391 | 3-{4-[(4-tert-butylbenzoyl)amino]-2-hydroxyphenyl}-5-cyclobutyl-N- |
| | cyclopropyl-1H-pyrazole-1-carboxamide |
| 392 | 3-{4-[(4-tert-butylbenzoyl)amino]-2-hydroxyphenyl}-5-cyclopentyl-N-isopropyl- |
| | 1H-pyrazole-1-carboxamide |
| 393 | 3-{4-[(4-tert-butylbenzoyl)amino]-2-hydroxyphenyl}-5-cyclopentyl-N-ethyl-1H- |
| | pyrazole-1-carboxamide |
| 394 | N-allyl-3-{4-[(4-tert-butylbenzoyl)amino]-2-hydroxyphenyl}-5-cyclopentyl-1H- |
| | pyrazole-1-carboxamide |
| 395 | 3-{4-[(4-tert-butylbenzoyl)amino]-2-hydroxyphenyl}-5-cyclopentyl-N-propyl- |
| | 1H-pyrazole-1-carboxamide |
| 396 | N-benzyl-3-{4-[(4-tert-butylbenzoyl)amino]-2-hydroxyphenyl}-5-cyclopentyl- |
| | 1H-pyrazole-1-carboxamide |
| 397 | 3-{4-[(4-tert-butylbenzoyl)amino]-2-hydroxyphenyl}-5-cyclopentyl-N-(2- |
| | phenylethyl)-1H-pyrazole-1-carboxamide |
| 398 | 3-{4-[(4-tert-butylbenzoyl)amino]-2-hydroxyphenyl}-5-cyclopentyl-N-(2- |
| | methylbenzyl)-1H-pyrazole-1-carboxamide |
| 399 | 3-{4-[(4-tert-butylbenzoyl)amino]-2-hydroxyphenyl}-5-cyclopentyl-N-(3- |
| | methylbenzyl)-1H-pyrazole-1-carboxamide |
| 400 | 3-{4-[(4-tert-butylbenzoyl)amino]-2-hydroxyphenyl}-5-cyclopentyl-N-(4- |
| | methylbenzyl)-1H-pyrazole-1-carboxamide |
| 401 | 3-{4-[(4-tert-butylbenzoyl)amino]-2-hydroxyphenyl}-N-(2-chlorobenzyl)-5- |
| | cyclopentyl-1H-pyrazole-1-carboxamide |
| 402 | 3-{4-[(4-tert-butylbenzoyl)amino]-2-hydroxyphenyl}-5-cyclopentyl-N-(4- |
| | fluorobenzyl)-1H-pyrazole-1-carboxamide |
| 403 | 3-{4-[(4-tert-butylbenzoyl)amino]-2-hydroxyphenyl}-5-cyclopentyl-N-(3,4- |
| | dichlorobenzyl)-1H-pyrazole-1-carboxamide |
| 404 | 3-{4-[(4-tert-butylbenzoyl)amino]-2-hydroxyphenyl}-5-cyclopentyl-N-(4- |
| | methoxybenzyl)-1H-pyrazole-1-carboxamide |
| 405 | N-(sec-butyl)-3-{4-[(4-tert-butylbenzoyl)amino]-2-hydroxyphenyl}-5- |
| | cyclopentyl-1H-pyrazole-1-carboxamide |
| 406 | 3-{4-[(4-tert-butylbenzoyl)amino]-2-hydroxyphenyl}-5-cyclopentyl-N-(2- |
| | fluorobenzyl)-1H-pyrazole-1-carboxamide |
| 407 | 3-{4-[(4-tert-butylbenzoyl)amino]-2-hydroxyphenyl}-5-cyclopentyl-N-(3- |
| | fluorobenzyl)-1H-pyrazole-1-carboxamide |
| 408 | 3-{4-[(4-tert-butylbenzoyl)amino]-2-hydroxyphenyl}-5-cyclopentyl-N- |
| | cyclopropyl-1H-pyrazole-1-carboxamide |
| 409 | 5-cyclopropyl-3-{2-hydroxy-4-[(4-methylbenzoyl)amino]phenyl}-N-isopropyl- |
| | 1H-pyrazole-1-carboxamide |
| 410 | 5-cyclopropyl-N-ethyl-3-{2-hydroxy-4-[(4-methylbenzoyl)amino]phenyl}-1H- |
| | pyrazole-1-carboxamide |
| 411 | N-allyl-5-cyclopropyl-3-{2-hydroxy-4-[(4-methylbenzoyl)amino]phenyl}-1H- |
| | pyrazole-1-carboxamide |
| 412 | 5-cyclopropyl-3-{2-hydroxy-4-[(4-methylbenzoyl)amino]phenyl}-N-propyl-1H- |
| | pyrazole-1-carboxamide |
| 413 | N-benzyl-5-cyclopropyl-3-{2-hydroxy-4-[(4-methylbenzoyl)amino]phenyl}-1H- |
| | pyrazole-1-carboxamide |
| 414 | 5-cyclopropyl-3-{2-hydroxy-4-[(4-methylbenzoyl)amino]phenyl}-N-(2- |
| | phenylethyl)-1H-pyrazole-1-carboxamide |
| 415 | 5-cyclopropyl-3-{2-hydroxy-4-[(4-methylbenzoyl)amino]phenyl}-N-(2- |
| | methylbenzyl)-1H-pyrazole-1-carboxamide |
| 416 | 5-cyclopropyl-3-{2-hydroxy-4-[(4-methylbenzoyl)amino]phenyl}-N-(3- |
| | methylbenzyl)-1H-pyrazole-1-carboxamide |
| 417 | 5-cyclopropyl-3-{2-hydroxy-4-[(4-methylbenzoyl)amino]phenyl}-N-(4- |
| | methylbenzyl)-1H-pyrazole-1-carboxamide |
| 418 | N-(2-chlorobenzyl)-5-cyclopropyl-3-{2-hydroxy-4-[(4- |
| | methylbenzoyl)amino]phenyl}-1H-pyrazole-1-carboxamide |
| 419 | 5-cyclopropyl-N-(4-fluorobenzyl)-3-{2-hydroxy-4-[(4- |
| | methylbenzoyl)amino]phenyl}-1H-pyrazole-1-carboxamide |
| 420 | 5-cyclopropyl-N-(3,4-dichlorobenzyl)-3-{2-hydroxy-4-[(4- |
| | methylbenzoyl)amino]phenyl}-1H-pyrazole-1-carboxamide |
| 421 | 5-cyclopropyl-3-{2-hydroxy-4-[(4-methylbenzoyl)amino]phenyl}-N-(4- |
| | methoxybenzyl)-1H-pyrazole-1-carboxamide |
| 422 | N-(sec-butyl)-5-cyclopropyl-3-{2-hydroxy-4-[(4-methylbenzoyl)amino]phenyl}- |
| | 1H-pyrazole-1-carboxamide |
| 423 | 5-cyclopropyl-N-(2-fluorobenzyl)-3-{2-hydroxy-4-[(4- |
| | methylbenzoyl)amino]phenyl}-1H-pyrazole-1-carboxamide |
| 424 | 5-cyclopropyl-N-(3-fluorobenzyl)-3-{2-hydroxy-4-[(4- |
| | methylbenzoyl)amino]phenyl}-1H-pyrazole-1-carboxamide |
| 425 | N,5-dicyclopropyl-3-{2-hydroxy-4-[(4-methylbenzoyl)amino]phenyl}-1H- |
| | pyrazole-1-carboxamide |
| 426 | 5-cyclobutyl-3-{2-hydroxy-4-[(4-methylbenzoyl)amino]phenyl}-N-isopropyl-1H- |
| | pyrazole-1-carboxamide |
| 427 | 5-cyclobutyl-N-ethyl-3-{2-hydroxy-4-[(4-methylbenzoyl)amino]phenyl}-1H- |
| | pyrazole-1-carboxamide |
| 428 | N-allyl-5-cyclobutyl-3-{2-hydroxy-4-[(4-methylbenzoyl)amino]phenyl}-1H- |
| | pyrazole-1-carboxamide |
| 429 | 5-cyclobutyl-3-{2-hydroxy-4-[(4-methylbenzoyl)amino]phenyl}-N-propyl-1H- |
| | pyrazole-1-carboxamide |
| 430 | N-benzyl-5-cyclobutyl-3-{2-hydroxy-4-[(4-methylbenzoyl)amino]phenyl}-1H- |
| | pyrazole-1-carboxamide |
| 431 | 5-cyclobutyl-3-{2-hydroxy-4-[(4-methylbenzoyl)amino]phenyl}-N-(2- |
| | phenylethyl)-1H-pyrazole-1-carboxamide |
| 432 | 5-cyclobutyl-3-{2-hydroxy-4-[(4-methylbenzoyl)amino]phenyl}-N-(2- |
| | methylbenzyl)-1H-pyrazole-1-carboxamide |
| 433 | 5-cyclobutyl-3-{2-hydroxy-4-[(4-methylbenzoyl)amino]phenyl}-N-(3- |
| | methylbenzyl)-1H-pyrazole-1-carboxamide |
| 434 | 5-cyclobutyl-3-{2-hydroxy-4-[(4-methylbenzoyl)amino]phenyl}-N-(4- |
| | methylbenzyl)-1H-pyrazole-1-carboxamide |
| 435 | N-(2-chlorobenzyl)-5-cyclobutyl-3-{2-hydroxy-4-[(4- |
| | methylbenzoyl)amino]phenyl}-1H-pyrazole-1-carboxamide |
| 436 | 5-cyclobutyl-N-(4-fluorobenzyl)-3-{2-hydroxy-4-[(4- |
| | methylbenzoyl)amino]phenyl}-1H-pyrazole-1-carboxamide |
| 437 | 5-cyclobutyl-N-(3,4-dichlorobenzyl)-3-{2-hydroxy-4-[(4- |
| | methylbenzoyl)amino]phenyl}-1H-pyrazole-1-carboxamide |
| 438 | 5-cyclobutyl-3-{2-hydroxy-4-[(4-methylbenzoyl)amino]phenyl}-N-4- |
| | methoxybenzyl)-1H-pyrazole-1-carboxamide |
| 439 | N-(sec-butyl)-5-cyclobutyl-3-{2-hydroxy-4-[(4-methylbenzoyl)amino]phenyl}- |
| | 1H-pyrazole-1-carboxamide |
| 440 | 5-cyclobutyl-N-(2-fluorobenzyl)-3-{2-hydroxy-4-[(4- |
| | methylbenzoyl)amino]phenyl}-1H-pyrazole-1-carboxamide |
| 441 | 5-cyclopentyl-3-{2-hydroxy-4-[(4-methylbenzoyl)amino]phenyl}-N-propyl-1H- |
| | pyrazole-1-carboxamide |
| 442 | 5-cyclobutyl-N-cyclopropyl-3-{2-hydroxy-4-[(4-methylbenzoyl)amino]phenyl}- |
| | 1H-pyrazole-1-carboxamide |
| 443 | 5-cyclopentyl-3-{2-hydroxy-4-[(4-methylbenzoyl)amino]phenyl}-N-isopropyl- |
| | 1H-pyrazole-1-carboxamide |
| 444 | 5-cyclopentyl-N-ethyl-3-{2-hydroxy-4-[(4-methylbenzoyl)amino]phenyl}-1H- |
| | pyrazole-1-carboxamide |
| 445 | N-allyl-5-cyclopentyl-3-{2-hydroxy-4-[(4-methylbenzoyl)amino]phenyl}-1H- |
| | pyrazole-1-carboxamide |
| 446 | 5-cyclopentyl-3-{2-hydroxy-4-[(4-methylbenzoyl)amino]phenyl}-N-propyl-1H- |
| | pyrazole-1-carboxamide |
| 447 | N-benzyl-5-cyclopentyl-3-{2-hydroxy-4-[(4-methylbenzoyl)amino]phenyl}-1H- |
| | pyrazole-1-carboxamide |
| 448 | 5-cyclopentyl-3-{2-hydroxy-4-[(4-methylbenzoyl)amino]phenyl}-N-(2- |
| | phenylethyl)-1H-pyrazole-1-carboxamide |
| 449 | 5-cyclopentyl-3-{2-hydroxy-4-[(4-methylbenzoyl)amino]phenyl}-N-(2- |
| | methylbenzyl)-1H-pyrazole-1-carboxamide |
| 450 | 5-cyclopentyl-3-{2-hydroxy-4-[(4-methylbenzoyl)amino]phenyl}-N-(3- |
| | methylbenzyl)-1H-pyrazole-1-carboxamide |
| 451 | 5-cyclopentyl-3-{2-hydroxy-4-[(4-methylbenzoyl)amino]phenyl}-N-(4- |
| | methylbenzyl)-1H-pyrazole-1-carboxamide |
| 452 | N-(2-chlorobenzyl)-5-cyclopentyl-3-{2-hydroxy-4-[(4- |
| | methylbenzoyl)amino]phenyl}-1H-pyrazole-1-carboxamide |
| 453 | 5-cyclopentyl-N-(4-fluorobenzyl)-3-{2-hydroxy-4-[(4- |
| | methylbenzoyl)amino]phenyl}-1H-pyrazole-1-carboxamide |
| 454 | 5-cyclopentyl-N-(3,4-dichlorobenzyl)-3-{2-hydroxy-4-[(4- |
| | methylbenzoyl)amino]phenyl}-1H-pyrazole-1-carboxamide |
| 455 | 5-cyclopentyl-3-{2-hydroxy-4-[(4-methylbenzoyl)amino]phenyl}-N-(4- |
| | methoxybenzyl)-1H-pyrazole-1-carboxamide |
| 456 | N-(sec-butyl)-5-cyclopentyl-3-{2-hydroxy-4-[(4-methylbenzoyl)amino]phenyl}- |
| | 1H-pyrazole-1-carboxamide |
| 457 | 5-cyclopentyl-N-(2-fluorobenzyl)-3-{2-hydroxy-4-[(4- |
| | methylbenzoyl)amino]phenyl}-1H-pyrazole-1-carboxamide |
| 458 | 5-cyclopentyl-N-(3-fluorobenzyl)-3-{2-hydroxy-4-[(4- |
| | methylbenzoyl)amino]phenyl}-1H-pyrazole-1-carboxamide |
| 459 | 5-cyclopentyl-N-cyclopropyl-3-{2-hydroxy-4-[(4-methylbenzoyl)amino]phenyl}- |
| | 1H-pyrazole-1-carboxamide |
| 460 | 5-cyclopropyl-3-{4-[(4-ethylbenzoyl)amino]-2-hydroxyphenyl}-N-isopropyl-1H- |
| | pyrazole-1-carboxamide |
| 461 | 5-cyclopropyl-N-ethyl-3-{4-[(4-ethylbenzoyl)amino]-2-hydroxyphenyl}-1H- |
| | pyrazole-1-carboxamide |
| 462 | N-allyl-5-cyclopropyl-3-{4-[(4-ethylbenzoyl)amino]-2-hydroxyphenyl}-1H- |
| | pyrazole-1-carboxamide |
| 463 | 5-cyclopropyl-3-{4-[(4-ethylbenzoyl)amino]-2-hydroxyphenyl}-N-propyl-1H- |
| | pyrazole-1-carboxamide |
| 464 | N-benzyl-5-cyclopropyl-3-{4-[(4-ethylbenzoyl)amino]-2-hydroxyphenyl}-1H- |
| | pyrazole-1-carboxamide |
| 465 | 5-cyclopropyl-3-{4-[(4-ethylbenzoyl)amino]-2-hydroxyphenyl}-N-(2- |
| | phenylethyl)-1H-pyrazole-1-carboxamide |
| 466 | 5-cyclopropyl-3-{4-[(4-ethylbenzoyl)amino]-2-hydroxyphenyl}-N-(2- |
| | methylbenzyl)-1H-pyrazole-1-carboxamide |
| 467 | 5-cyclopropyl-3-{4-[(4-ethylbenzoyl)amino]-2-hydroxyphenyl}-N-(3- |
| | methylbenzyl)-1H-pyrazole-1-carboxamide |
| 468 | 5-cyclobutyl-3-{4-[(4-ethylbenzoyl)amino]-2-hydroxyphenyl}-N-isopropyl-1H- |
| | pyrazole-1-carboxamide |
| 469 | N-(2-chlorobenzyl)-5-cyclopropyl-3-{4-[(4-ethylbenzoyl)amino]-2- |
| | hydroxyphenyl}-1H-pyrazole-1-carboxamide |
| 470 | 5-cyclopropyl-3-{4-[(4-ethylbenzoyl)amino]-2-hydroxyphenyl}-N-(4- |
| | fluorobenzyl)-1H-pyrazole-1-carboxamide |
| 471 | 5-cyclopropyl-N-(3,4-dichlorobenzyl)-3-{4-[(4-ethylbenzoyl)amino]-2- |
| | hydroxyphenyl}-1H-pyrazole-1-carboxamide |
| 472 | 5-cyclopropyl-3-{4-[(4-ethylbenzoyl)amino]-2-hydroxyphenyl}-N-(4- |
| | methoxybenzyl)-1H-pyrazole-1-carboxamide |
| 473 | N-(sec-butyl)-5-cyclopropyl-3-{4-[(4-ethylbenzoyl)amino]-2-hydroxyphenyl}- |
| | 1H-pyrazole-1-carboxamide |
| 474 | 5-cyclopropyl-3-{4-[(4-ethylbenzoyl)amino]-2-hydroxyphenyl}-N-(2- |
| | fluorobenzyl)-1H-pyrazole-1-carboxamide |
| 475 | 5-cyclopropyl-3-{4-[(4-ethylbenzoyl)amino]-2-hydroxyphenyl}-N-(3- |
| | fluorobenzyl)-1H-pyrazole-1-carboxamide |
| 476 | N,5-dicyclopropyl-3-{4-[(4-ethylbenzoyl)amino]-2-hydroxyphenyl}-1H-pyrazole- |
| | 1-carboxamide |
| 477 | 5-cyclobutyl-3-{4-[(4-ethylbenzoyl)amino]-2-hydroxyphenyl}-N-isopropyl-1H- |
| | pyrazole-1-carboxamide |
| 478 | 5-cyclobutyl-N-ethyl-3-{4-[(4-ethylbenzoyl)amino]-2-hydroxyphenyl}-1H- |
| | pyrazole-1-carboxamide |
| 479 | N-allyl-5-cyclobutyl-3-{4-[(4-ethylbenzoyl)amino]-2-hydroxyphenyl}-1H- |
| | pyrazole-1-carboxamide |
| 480 | 5-cyclobutyl-3-{4-[(4-ethylbenzoyl)amino]-2-hydroxyphenyl}-N-propyl-1H- |
| | pyrazole-1-carboxamide |
| 481 | N-benzyl-5-cyclobutyl-3-{4-[(4-ethylbenzoyl)amino]-2-hydroxyphenyl}-1H- |
| | pyrazole-1-carboxamide |
| 482 | 5-cyclobutyl-3-{4-[(4-ethylbenzoyl)amino]-2-hydroxyphenyl}-N-(2-phenylethyl)- |
| | 1H-pyrazole-1-carboxamide |
| 483 | 5-cyclobutyl-3-{4-[(4-ethylbenzoyl)amino]-2-hydroxyphenyl}-N-(2- |
| | methylbenzyl)-1H-pyrazole-1-carboxamide |
| 484 | 5-cyclobutyl-3-{4-[(4-ethylbenzoyl)amino]-2-hydroxyphenyl}-N-(3- |
| | methylbenzyl)-1H-pyrazole-1-carboxamide |
| 485 | 5-cyclobutyl-3-{4-[(4-ethylbenzoyl)amino]-2-hydroxyphenyl}-N-(4- |
| | methylbenzyl)-1H-pyrazole-1-carboxamide |
| 486 | N-(2-chlorobenzyl)-5-cyclobutyl-3-{4-[(4-ethylbenzoyl)amino]-2- |
| | hydroxyphenyl}-1H-pyrazole-1-carboxamide |
| 487 | 5-cyclobutyl-3-{4-[(4-ethylbenzoyl)amino]-2-hydroxyphenyl}-N-(4- |
| | fluorobenzyl)-1H-pyrazole-1-carboxamide |
| 488 | 5-cyclobutyl-N-(3,4-dichlorobenzyl)-3-{4-[(4-ethylbenzoyl)amino]-2- |
| | hydroxyphenyl}-1H-pyrazole-1-carboxamide |
| 489 | 5-cyclobutyl-3-{4-[(4-ethylbenzoyl)amino]-2-hydroxyphenyl}-N-(4- |
| | methoxybenzyl)-1H-pyrazole-1-carboxamide |
| 490 | N-(sec-butyl)-5-cyclobutyl-3-{4-[(4-ethylbenzoyl)amino]-2-hydroxyphenyl}-1H- |
| | pyrazole-1-carboxamide |
| 491 | 5-cyclobutyl-3-{4-[(4-ethylbenzoyl)amino]-2-hydroxyphenyl}-N-(2- |
| | fluorobenzyl)-1H-pyrazole-1-carboxamide |
| 492 | 5-cyclobutyl-3-{4-[(4-ethylbenzoyl)amino]-2-hydroxyphenyl}-N-(3- |
| | fluorobenzyl)-1H-pyrazole-1-carboxamide |
| 493 | 5-cyclobutyl-N-cyclopropyl-3-{4-[(4-ethylbenzoyl)amino]-2-hydroxyphenyl}- |
| | 1H-pyrazole-1-carboxamide |
| 494 | 5-cyclopentyl-3-{4-[(4-ethylbenzoyl)amino]-2-hydroxyphenyl}-N-isopropyl-1H- |
| | pyrazole-1-carboxamide |
| 495 | 5-cyclopentyl-N-ethyl-3-{4-[(4-ethylbenzoyl)amino]-2-hydroxyphenyl}-1H- |
| | pyrazole-1-carboxamide |
| 496 | N-allyl-5-cyclopentyl-3-{4-[(4-ethylbenzoyl)amino]-2-hydroxyphenyl}-1H- |
| | pyrazole-1-carboxamide |
| 497 | 5-cyclopentyl-3-{4-[(4-ethylbenzoyl)amino]-2-hydroxyphenyl}-N-propyl-1H- |
| | pyrazole-1-carboxamide |
| 498 | N-benzyl-5-cyclopentyl-3-{4-[(4-ethylbenzoyl)amino]-2-hydroxyphenyl}-1H- |
| | pyrazole-1-carboxamide |
| 499 | 5-cyclopentyl-3-{4-[(4-ethylbenzoyl)amino]-2-hydroxyphenyl}-N-(2- |
| | phenylethyl)-1H-pyrazole-1-carboxamide |
| 500 | 5-cyclopentyl-3-{4-[(4-ethylbenzoyl)amino]-2-hydroxyphenyl}-N-(2- |
| | methylbenzyl)-1H-pyrazole-1-carboxamide |
| 501 | 5-cyclopentyl-3-{4-[(4-ethylbenzoyl)amino]-2-hydroxyphenyl}-N-(3- |
| | methylbenzyl)-1H-pyrazole-1-carboxamide |
| 502 | 5-cyclopentyl-3-{4-[(4-ethylbenzoyl)amino]-2-hydroxyphenyl}-N-(4- |
| | methylbenzyl)-1H-pyrazole-1-carboxamide |
| 503 | N-(2-chlorobenzyl)-5-cyclopentyl-3-{4-[(4-ethylbenzoyl)amino]-2- |
| | hydroxyphenyl}-1H-pyrazole-1-carboxamide |
| 504 | 5-cyclopentyl-3-{4-[(4-ethylbenzoyl)amino]-2-hydroxyphenyl}-N-(4- |
| | fluorobenzyl)-1H-pyrazole-1-carboxamide |
| 505 | 5-cyclopentyl-N-(3,4-dichlorobenzyl)-3-{4-[(4-ethylbenzoyl)amino]-2- |
| | hydroxyphenyl}-1H-pyrazole-1-carboxamide |
| 506 | 5-cyclopentyl-3-{4-[(4-ethylbenzoyl)amino]-2-hydroxyphenyl}-N-(4- |
| | methoxybenzyl)-1H-pyrazole-1-carboxamide |
| 507 | N-(sec-butyl)-5-cyclopentyl-3-{4-[(4-ethylbenzoyl)amino]-2-hydroxyphenyl}- |
| | 1H-pyrazole-1-carboxamide |
| 508 | 5-cyclopentyl-3-{4-[(4-ethylbenzoyl)amino]-2-hydroxyphenyl}-N-(2- |
| | fluorobenzyl)-1H-pyrazole-1-carboxamide |
| 509 | 5-cyclopentyl-3-{4-[(4-ethylbenzoyl)amino]-2-hydroxyphenyl}-N-(3- |
| | fluorobenzyl)-1H-pyrazole-1-carboxamide |
| 510 | 5-cyclopentyl-N-cyclopropyl-3-{4-[(4-ethylbenzoyl)amino]-2-hydroxyphenyl}- |
| | 1H-pyrazole-1-carboxamide |
| 511 | 5-cyclopropyl-3-[2-hydroxy-4-(isobutyrylamino)phenyl]-N-isopropyl-1H- |
| | pyrazole-1-carboxamide |
| 512 | 5-cyclopropyl-N-ethyl-3-[2-hydroxy-4-(isobutyrylamino)phenyl]-1H-pyrazole-1- |
| | carboxamide |
| 513 | N-allyl-5-cyclopropyl-3-[2-hydroxy-4-(isobutyrylamino)phenyl]-1H-pyrazole-1- |
| | carboxamide |
| 514 | 5-cyclopropyl-3-[2-hydroxy-4-(isobutyrylamino)phenyl]-N-propyl-1H-pyrazole- |
| | 1-carboxamide |
| 515 | N-benzyl-5-cyclopropyl-3-[2-hydroxy-4-(isobutyrylamino)phenyl]-1H-pyrazole- |
| | 1-carboxamide |
| 516 | 5-cyclopropyl-3-[2-hydroxy-4-(isobutyrylamino)phenyl]-N-(2-phenylethyl)-1H- |
| | pyrazole-1-carboxamide |
| 517 | 5-cyclopropyl-3-[2-hydroxy-4-(isobutyrylamino)phenyl]-N-(2-methylbenzyl)-1H- |
| | pyrazole-1-carboxamide |
| 518 | 5-cyclopropyl-3-[2-hydroxy-4-(isobutyrylamino)phenyl]-N-(3-methylbenzyl)-1H- |
| | pyrazole-1-carboxamide |
| 519 | 5-cyclopropyl-3-[2-hydroxy-4-(isobutyrylamino)phenyl]-N-(4-methylbenzyl)-1H- |
| | pyrazole-1-carboxamide |
| 520 | N-(2-chlorobenzyl)-5-cyclopropyl-3-[2-hydroxy-4-(isobutyrylamino)phenyl]-1H- |
| | pyrazole-1-carboxamide |
| 521 | 5-cyclopropyl-N-(4-fluorobenzyl)-3-[2-hydroxy-4-(isobutyrylamino)phenyl]-1H- |
| | pyrazole-1-carboxamide |
| 522 | 5-cyclopropyl-N-(3,4-dichlorobenzyl)-3-[2-hydroxy-4-(isobutyrylamino)phenyl]- |
| | 1H-pyrazole-1-carboxamide |
| 523 | 5-cyclopropyl-3-[2-hydroxy-4-(isobutyrylamino)phenyl]-N-(4-methoxybenzyl)- |
| | 1H-pyrazole-1-carboxamide |
| 524 | N-(sec-butyl)-5-cyclopropyl-3-[2-hydroxy-4-(isobutyrylamino)phenyl]-1H- |
| | pyrazole-1-carboxamide |
| 525 | 5-cyclopropyl-N-(2-fluorobenzyl)-3-[2-hydroxy-4-(isobutyrylamino)phenyl]-1H- |
| | pyrazole-1-carboxamide |
| 526 | 5-cyclopropyl-N-(3-fluorobenzyl)-3-[2-hydroxy-4-(isobutyrylamino)phenyl]-1H- |
| | pyrazole-1-carboxamide |
| 527 | N,5-dicyclopropyl-3-[2-hydroxy-4-(isobutyrylamino)phenyl]-1H-pyrazole-1- |
| | carboxamide |
| 528 | 5-cyclobutyl-3-[2-hydroxy-4-(isobutyrylamino)phenyl]-N-isopropyl-1H-pyrazole- |
| | 1-carboxamide |
| 529 | 5-cyclobutyl-N-ethyl-3-[2-hydroxy-4-(isobutyrylamino)phenyl]-1H-pyrazole-1- |
| | carboxamide |
| 530 | N-allyl-5-cyclobutyl-3-[2-hydroxy-4-(isobutyrylamino)phenyl]-1H-pyrazole-1- |
| | carboxamide |
| 531 | 5-cyclobutyl-3-[2-hydroxy-4-(isobutyrylamino)phenyl]-N-propyl-1H-pyrazole-1- |
| | carboxamide |
| 532 | N-benzyl-5-cyclobutyl-3-[2-hydroxy-4-(isobutyrylamino)phenyl]-1H-pyrazole-1- |
| | carboxamide |
| 533 | 5-cyclobutyl-3-[2-hydroxy-4-(isobutyrylamino)phenyl]-N-(2-phenylethyl)-1H- |
| | pyrazole-1-carboxamide |
| 534 | 5-cyclobutyl-3-[2-hydroxy-4-(isobutyrylamino)phenyl]-N-(2-methylbenzyl)-1H- |
| | pyrazole-1-carboxamide |
| 535 | 5-cyclobutyl-3-[2-hydroxy-4-(isobutyrylamino)phenyl]-N-(3-methylbenzyl)-1H- |
| | pyrazole-1-carboxamide |
| 536 | 5-cyclobutyl-3-[2-hydroxy-4-(isobutyrylamino)phenyl]-N-(4-methylbenzyl)-1H- |
| | pyrazole-1-carboxamide |
| 537 | N-(2-chlorobenzyl)-5-cyclobutyl-3-[2-hydroxy-4-(isobutyrylamino)phenyl]-1H- |
| | pyrazole-1-carboxamide |
| 538 | 5-cyclobutyl-N-(4-fluorobenzyl)-3-[2-hydroxy-4-(isobutyrylamino)phenyl]-1H- |
| | pyrazole-1-carboxamide |
| 539 | 5-cyclobutyl-N-(3,4-dichlorobenzyl)-3-[2-hydroxy-4-(isobutyrylamino)phenyl]- |
| | 1H-pyrazole-1-carboxamide |
| 540 | 5-cyclobutyl-3-[2-hydroxy-4-(isobutyrylamino)phenyl]-N-(4-methoxybenzyl)- |
| | 1H-pyrazole-1-carboxamide |
| 541 | N-(sec-butyl)-5-cyclobutyl-3-[2-hydroxy-4-(isobutyrylamino)phenyl]-1H- |
| | pyrazole-1-carboxamide |
| 542 | 5-cyclobutyl-N-(2-fluorobenzyl)-3-[2-hydroxy-4-(isobutyrylamino)phenyl]-1H- |
| | pyrazole-1-carboxamide |
| 543 | 5-cyclobutyl-N-(3-fluorobenzyl)-3-[2-hydroxy-4-(isobutyrylamino)phenyl]-1H- |
| | pyrazole-1-carboxamide |
| 544 | 5-cyclobutyl-N-cyclopropyl-3-[2-hydroxy-4-(isobutyrylamino)phenyl]-1H- |
| | pyrazole-1-carboxamide |
| 545 | 5-cyclopentyl-3-[2-hydroxy-4-(isobutyrylamino)phenyl]-N-isopropyl-1H- |
| | pyrazole-1-carboxamide |
| 546 | 5-cyclopentyl-N-ethyl-3-[2-hydroxy-4-(isobutyrylamino)phenyl]-1H-pyrazole-1- |
| | carboxamide |
| 547 | N-allyl-5-cyclopentyl-3-[2-hydroxy-4-(isobutyrylamino)phenyl]-1H-pyrazole-1- |
| | carboxamide |
| 548 | 5-cyclopentyl-3-[2-hydroxy-4-(isobutyrylamino)phenyl]-N-propyl-1H-pyrazole- |
| | 1-carboxamide |
| 549 | N-benzyl-5-cyclopentyl-3-[2-hydroxy-4-(isobutyrylamino)phenyl]-1H-pyrazole- |
| | 1-carboxamide |
| 550 | 5-cyclopentyl-3-[2-hydroxy-4-(isobutyrylamino)phenyl]-N-(2-phenylethyl)-1H- |
| | pyrazole-1-carboxamide |
| 551 | 5-cyclopentyl-3-[2-hydroxy-4-(isobutyrylamino)phenyl]-N-(2-methylbenzyl)-1H- |
| | pyrazole-1-carboxamide |
| 552 | 5-cyclopentyl-3-[2-hydroxy-4-(isobutyrylamino)phenyl]-N-(3-methylbenzyl)-1H- |
| | pyrazole-1-carboxamide |
| 553 | 5-cyclopentyl-3-[2-hydroxy-4-(isobutyrylamino)phenyl]-N-(4-methylbenzyl)-1H- |
| | pyrazole-1-carboxamide |
| 554 | N-(2-chlorobenzyl)-5-cyclopentyl-3-[2-hydroxy-4-(isobutyrylamino)phenyl]-1H- |
| | pyrazole-1-carboxamide |
| 555 | 5-cyclopentyl-N-(4-fluorobenzyl)-3-[2-hydroxy-4-(isobutyrylamino)phenyl]-1H- |
| | pyrazole-1-carboxamide |
| 556 | 5-cyclopentyl-N-(3,4-dichlorobenzyl)-3-[2-hydroxy-4-(isobutyrylamino)phenyl]- |
| | 1H-pyrazole-1-carboxamide |
| 557 | 5-cyclopentyl-3-[2-hydroxy-4-(isobutyrylamino)phenyl]-N-(4-methoxybenzyl)- |
| | 1H-pyrazole-1-carboxamide |
| 558 | N-(sec-butyl)-5-cyclopentyl-3-[2-hydroxy-4-(isobutyrylamino)phenyl]-1H- |
| | pyrazole-1-carboxamide |
| 559 | 5-cyclopentyl-N-(2-fluorobenzyl)-3-[2-hydroxy-4-(isobutyrylamino)phenyl]-1H- |
| | pyrazole-1-carboxamide |
| 560 | 5-cyclopentyl-N-(3-fluorobenzyl)-3-[2-hydroxy-4-(isobutyrylamino)phenyl]-1H- |
| | pyrazole-1-carboxamide |
| 561 | 5-cyclopentyl-N-cyclopropyl-3-[2-hydroxy-4-(isobutyrylamino)phenyl]-1H- |
| | pyrazole-1-carboxamide |
| 562 | 3-[4-(acetylamino)-2-hydroxyphenyl]-5-cyclopropyl-N-isopropyl-1H-pyrazole-1- |
| | carboxamide |
| 563 | 3-[4-(acetylamino)-2-hydroxyphenyl]-5-cyclopropyl-N-ethyl-1H-pyrazole-1- |
| | carboxamide |
| 564 | 3-[4-(acetylamino)-2-hydroxyphenyl]-N-allyl-5-cyclopropyl-1H-pyrazole-1- |
| | carboxamide |
| 565 | 3-[4-(acetylamino)-2-hydroxyphenyl]-5-cyclopropyl-N-propyl-1H-pyrazole-1- |
| | carboxamide |
| 566 | 3-[4-(acetylamino)-2-hydroxyphenyl]-5-cyclopropyl-N-(2-phenylethyl)-1H- |
| | pyrazole-1-carboxamide |
| 567 | 3-[4-(acetylamino)-2-hydroxyphenyl]-5-cyclopropyl-N-(2-phenylethyl)-1H- |
| | pyrazole-1-carboxamide |
| 568 | 3-[4-(acetylamino)-2-hydroxyphenyl]-5-cyclopropyl-N-(2-methylbenzyl)-1H- |
| | pyrazole-1-carboxamide |
| 569 | 3-[4-(acetylamino)-2-hydroxyphenyl]-5-cyclopropyl-N-(3-methylbenzyl)-1H- |
| | pyrazole-1-carboxamide |
| 570 | 3-[4-(acetylamino)-2-hydroxyphenyl]-5-cyclopropyl-N-(4-methylbenzyl)-1H- |
| | pyrazole-1-carboxamide |
| 571 | 3-[4-(acetylamino)-2-hydroxyphenyl]-N-(2-chlorobenzyl)-5-cyclopropyl-1H- |
| | pyrazole-1-carboxamide |
| 572 | 3-[4-(acetylamino)-2-hydroxyphenyl]-5-cyclopropyl-N-(4-fluorobenzyl)-1H- |
| | pyrazole-1-carboxamide |
| 573 | 3-[4-(acetylamino)-2-hydroxyphenyl]-5-cyclopropyl-N-(3,4-dichlorobenzyl)-1H- |
| | pyrazole-1-carboxamide |
| 574 | 3-[4-(acetylamino)-2-hydroxyphenyl]-5-cyclopropyl-N-(4-methoxybenzyl)-1H- |
| | pyrazole-1-carboxamide |
| 575 | 3-[4-(acetylamino)-2-hydroxyphenyl]-N-(sec-butyl)-5-cyclopropyl-1H-pyrazole- |
| | 1-carboxamide |
| 576 | 3-[4-(acetylamino)-2-hydroxyphenyl]-5-cyclopropyl-N-(2-fluorobenzyl)-1H- |
| | pyrazole-1-carboxamide |
| 577 | 3-[4-(acetylamino)-2-hydroxyphenyl]-5-cyclopropyl-N-(3-fluorobenzyl)-1H- |
| | pyrazole-1-carboxamide |
| 578 | 3-[4-(acetylamino)-2-hydroxyphenyl]-N,5-dicyclopropyl-1H-pyrazole-1- |
| | carboxamide |
| 579 | 3-[4-(acetylamino)-2-hydroxyphenyl]-5-cyclobutyl-N-isopropyl-1H-pyrazole-1- |
| | carboxamide |
| 580 | 3-[4-(acetylamino)-2-hydroxyphenyl]-5-cyclobutyl-N-ethyl-1H-pyrazole-1- |
| | carboxamide |
| 581 | 3-[4-(acetylamino)-2-hydroxyphenyl]-N-allyl-5-cyclobutyl-1H-pyrazole-1- |
| | carboxamide |
| 582 | 3-[4-(acetylamino)-2-hydroxyphenyl]-5-cyclobutyl-N-propyl-1H-pyrazole-1- |
| | carboxamide |
| 583 | 3-[4-(acetylamino)-2-hydroxyphenyl]-N-benzyl-5-cyclobutyl-1H-pyrazole-1- |
| | carboxamide |
| 584 | 3-[4-(acetylamino)-2-hydroxyphenyl]-5-cyclobutyl-N-(2-phenylethyl)-1H- |
| | pyrazole-1-carboxamide |
| 585 | 3-[4-(acetylamino)-2-hydroxyphenyl]-5-cyclobutyl-N-(2-methylbenzyl)-1H- |
| | pyrazole-1-carboxamide |
| 586 | 3-[4-(acetylamino)-2-hydroxyphenyl]-5-cyclobutyl-N-(3-methylbenzyl)-1H- |
| | pyrazole-1-carboxamide |
| 587 | 3-[4-(acetylamino)-2-hydroxyphenyl]-5-cyclobutyl-N-(4-methylbenzyl)-1H- |
| | pyrazole-1-carboxamide |
| 588 | 3-[4-(acetylamino)-2-hydroxyphenyl]-N-(2-chlorobenzyl)-5-cyclobutyl-1H- |
| | pyrazole-1-carboxamide |
| 589 | 3-[4-(acetylamino)-2-hydroxyphenyl]-5-cyclobutyl-N-(4-fluorobenzyl)-1H- |
| | pyrazole-1-carboxamide |
| 590 | 3-[4-(acetylamino)-2-hydroxyphenyl]-5-cyclobutyl-N-(3,4-dichlorobenzyl)-1H- |
| | pyrazole-1-carboxamide |
| 591 | 3-[4-(acetylamino)-2-hydroxyphenyl]-5-cyclobutyl-N-(4-methoxybenzyl)-1H- |
| | pyrazole-1-carboxamide |
| 592 | 3-[4-(acetylamino)-2-hydroxyphenyl]-N-(sec-butyl)-5-cyclobutyl-1H-pyrazole-1- |
| | carboxamide |
| 593 | 3-[4-(acetylamino)-2-hydroxyphenyl]-5-cyclobutyl-N-(2-fluorobenzyl)-1H- |
| | pyrazole-1-carboxamide |
| 594 | 3-[4-(acetylamino)-2-hydroxyphenyl]-5-cyclobutyl-N-(3-fluorobenzyl)-1H- |
| | pyrazole-1-carboxamide |
| 595 | 3-[4-(acetylamino)-2-hydroxyphenyl]-5-cyclobutyl-N-cyclopropyl-1H-pyrazole- |
| | 1-carboxamide |
| 596 | 3-[4-(acetylamino)-2-hydroxyphenyl]-5-cyclopentyl-N-isopropyl-1H-pyrazole-1- |
| | carboxamide |
| 597 | 3-[4-(acetylamino)-2-hydroxyphenyl]-5-cyclopentyl-N-ethyl-1H-pyrazole-1- |
| | carboxamide |
| 598 | 3-[4-(acetylamino)-2-hydroxyphenyl]-N-allyl-5-cyclopentyl-1H-pyrazole-1- |
| | carboxamide |
| 599 | 3-[4-(acetylamino)-2-hydroxyphenyl]-5-cyclopentyl-N-propyl-1H-pyrazole-1- |
| | carboxamide |
| 600 | 3-[4-(acetylamino)-2-hydroxyphenyl]-N-benzyl-5-cyclopentyl-1H-pyrazole-1- |
| | carboxamide |
| 601 | 3-[4-(acetylamino)-2-hydroxyphenyl]-5-cyclopentyl-N-(2-phenylethyl)-1H- |
| | pyrazole-1-carboxamide |
| 602 | 3-[4-(acetylamino)-2-hydroxyphenyl]-5-cyclopentyl-N-(2-methylbenzyl)-1H- |
| | pyrazole-1-carboxamide |
| 603 | 3-[4-(acetylamino)-2-hydroxyphenyl]-5-cyclopentyl-N-(3-methylbenzyl)-1H- |
| | pyrazole-1-carboxamide |
| 604 | 3-[4-(acetylamino)-2-hydroxyphenyl]-5-cyclopentyl-N-(4-methylbenzyl)-1H- |
| | pyrazole-1-carboxamide |
| 605 | 3-[4-(acetylamino)-2-hydroxyphenyl]-N-(2-chlorobenzyl)-5-cyclopentyl-1H- |
| | pyrazole-1-carboxamide |
| 606 | 3-[4-(acetylamino)-2-hydroxyphenyl]-5-cyclopentyl-N-(4-fluorobenzyl)-1H- |
| | pyrazole-1-carboxamide |
| 607 | 3-[4-(acetylamino)-2-hydroxyphenyl]-5-cyclopentyl-N-(3,4-dichlorobenzyl)-1H- |
| | pyrazole-1-carboxamide |
| 608 | 3-[4-(acetylamino)-2-hydroxyphenyl]-5-cyclopentyl-N-(4-methoxybenzyl)-1H- |
| | pyrazole-1-carboxamide |
| 609 | 3-[4-(acetylamino)-2-hydroxyphenyl]-N-(sec-butyl)-5-cyclopentyl-1H-pyrazole- |
| | 1-carboxamide |
| 610 | 3-[4-(acetylamino)-2-hydroxyphenyl]-5-cyclopentyl-N-(2-fluorobenzyl)-1H- |
| | pyrazole-1-carboxamide |
| 611 | 3-[4-(acetylamino)-2-hydroxyphenyl]-5-cyclopentyl-N-(3-fluorobenzyl)-1H- |
| | pyrazole-1-carboxamide |
| 612 | 3-[4-(acetylamino)-2-hydroxyphenyl]-5-cyclopentyl-N-cyclopropyl-1H-pyrazole- |
| | 1-carboxamide |
| 613 | 5-cyclopropyl-3-{2-hydroxy-4-[(3-methylbutanoyl)amino]phenyl}-N-isopropyl- |
| | 1H-pyrazole-1-carboxamide |
| 614 | 5-cyclopropyl-N-ethyl-3-{2-hydroxy-4-[(3-methylbutanoyl)amino]phenyl}-1H- |
| | pyrazole-1-carboxamide |
| 615 | N-allyl-5-cyclopropyl-3-{2-hydroxy-4-[(3-methylbutanoyl)amino]phenyl}-1H- |
| | pyrazole-1-carboxamide |
| 616 | 5-cyclopropyl-3-{2-hydroxy-4-[(3-methylbutanoyl)amino]phenyl}-N-propyl-1H- |
| | pyrazole-1-carboxamide |
| 617 | N-benzyl-5-cyclopropyl-3-{2-hydroxy-4-[(3-methylbutanoyl)amino]phenyl}-1H- |
| | pyrazole-1-carboxamide |
| 618 | 5-cyclopropyl-3-{2-hydroxy-4-[(3-methylbutanoyl)amino]phenyl}-N-(2- |
| | phenylethyl)-1H-pyrazole-1-carboxamide |
| 619 | 5-cyclopropyl-3-{2-hydroxy-4-[(3-methylbutanoyl)amino]phenyl}-N-(2- |
| | methylbenzyl)-1H-pyrazole-1-carboxamide |
| 620 | 5-cyclopropyl-3-{2-hydroxy-4-[(3-methylbutanoyl)amino]phenyl}-N-(3- |
| | methylbenzyl)-1H-pyrazole-1-carboxamide |
| 621 | 5-cyclopropyl-3-{2-hydroxy-4-[(3-methylbutanoyl)amino]phenyl}-N-(4- |
| | methylbenzyl)-1H-pyrazole-1-carboxamide |
| 622 | N-(2-chlorobenzyl)-5-cyclopropyl-3-{2-hydroxy-4-[(3- |
| | methylbutanoyl)amino]phenyl}-1H-pyrazole-1-carboxamide |
| 623 | 5-cyclopropyl-N-(4-fluorobenzyl)-3-{2-hydroxy-4-[(3- |
| | methylbutanoyl)amino]phenyl}-1H-pyrazole-1-carboxamide |
| 624 | 5-cyclopropyl-N-(3,4-dichlorobenzyl)-3-{2-hydroxy-4-[(3- |
| | methylbutanoyl)amino]phenyl}-1H-pyrazole-1-carboxamide |
| 625 | 5-cyclopropyl-3-{2-hydroxy-4-[(3-methylbutanoyl)amino]phenyl}-N-(4- |
| | methoxybenzyl)-1H-pyrazole-1-carboxamide |
| 626 | N-(sec-butyl)-5-cyclopropyl-3-{2-hydroxy-4-[(3-methylbutanoyl)amino]phenyl}- |
| | 1H-pyrazole-1-carboxamide |
| 627 | 5-cyclopropyl-N-(2-fluorobenzyl)-3-{2-hydroxy-4-[(3- |
| | methylbutanoyl)amino]phenyl}-1H-pyrazole-1-carboxamide |
| 628 | 5-cyclopropyl-N-(3-fluorobenzyl)-3-{2-hydroxy-4-[(3- |
| | methylbutanoyl)amino]phenyl}-1H-pyrazole-1-carboxamide |
| 629 | N,5-dicyclopropyl-3-{2-hydroxy-4-[(3-methylbutanoyl)amino]phenyl}-1H- |
| | pyrazole-1-carboxamide |
| 630 | 5-cyclobutyl-N-ethyl-3-{2-hydroxy-4-[(3-methylbutanoyl)amino]phenyl}-1H- |
| | pyrazole-1-carboxamide |
| 631 | 5-cyclobutyl-N-ethyl-3-{2-hydroxy-4-[(3-methylbutanoyl)amino]phenyl}-1H- |
| | pyrazole-1-carboxamide |
| 632 | N-allyl-5-cyclobutyl-3-{2-hydroxy-4-[(3-methylbutanoyl)amino]phenyl}-1H- |
| | pyrazole-1-carboxamide |
| 633 | 5-cyclobutyl-3-{2-hydroxy-4-[(3-methylbutanoyl)amino]phenyl}-N-propyl-1H- |
| | pyrazole-1-carboxamide |
| 634 | N-benzyl-5-cyclobutyl-3-{2-hydroxy-4-[(3-methylbutanoyl)amino]Phenyl}-1H- |
| | pyrazole-1-carboxamide |
| 635 | 5-cyclobutyl-3-{2-hydroxy-4-[(3-methylbutanoyl)amino]phenyl}-N-(2- |
| | phenylethyl)-1H-pyrazole-1-carboxamide |
| 636 | 5-cyclobutyl-3-{2-hydroxy-4-[(3-methylbutanoyl)amino]phenyl}-N-(2- |
| | methylbenzyl)-1H-pyrazole-1-carboxamide |
| 637 | 5-cyclobutyl-3-{2-hydroxy-4-[(3-methylbutanoyl)amino]phenyl}-N-(3- |
| | methylbenzyl)-1H-pyrazole-1-carboxamide |
| 638 | 5-cyclobutyl-3-{2-hydroxy-4-[(3-methylbutanoyl)amino]phenyl}-N-(4- |
| | methylbenzyl)-1H-pyrazole-1-carboxamide |
| 639 | N-(2-chlorobenzyl)-5-cyclobutyl-3-{2-hydroxy-4-[(3- |
| | methylbutanoyl)amino]phenyl}-1H-pyrazole-1-carboxamide |
| 640 | 5-cyclobutyl-N-(4-fluorobenzyl)-3-{2-hydroxy-4-[(3- |
| | methylbutanoyl)amino]phenyl}-1H-pyrazole-1-carboxamide |
| 641 | 5-cyclobutyl-N-(3,4-dichlorobenzyl)-3-{2-hydroxy-4-[(3- |
| | methylbutanoyl)amino]phenyl}-1H-pyrazole-1-carboxamide |
| 642 | 5-cyclobutyl-3-{2-hydroxy-4-[(3-methylbutanoyl)amino]phenyl}-N-(4- |
| | methoxybenzyl)-1H-pyrazole-1-carboxamide |
| 643 | N-(sec-butyl)-5-cyclobutyl-3-{2-hydroxy-4-[(3-methylbutanoyl)amino]phenyl}- |
| | 1H-pyrazole-1-carboxamide |
| 644 | 5-cyclobutyl-N-(2-fluorobenzyl)-3-{2-hydroxy-4-[(3- |
| | methylbutanoyl)amino]phenyl}-1H-pyrazole-1-carboxamide |
| 645 | 5-cyclobutyl-N-(3-fluorobenzyl)-3-{2-hydroxy-4-[(3- |
| | methylbutanoyl)amino]phenyl}-1H-pyrazole-1-carboxamide |
| 646 | 5-cyclobutyl-N-cyclopropyl-3-{2-hydroxy-4-[(3-methylbutanoyl)amino]phenyl}- |
| | 1H-pyrazole-1-carboxamide |
| 647 | 5-cyclopentyl-3-{2-hydroxy-4-[(3-methylbutanoyl)amino]phenyl}-N-isopropyl- |
| | 1H-pyrazole-1-carboxamide |
| 648 | 5-cyclopentyl-N-ethyl-3-{2-hydroxy-4-[(3-methylbutanoyl)amino]phenyl}-1H- |
| | pyrazole-1-carboxamide |
| 649 | N-allyl-5-cyclopentyl-3-{2-hydroxy-4-[(3-methylbutanoyl)amino]phenyl}-1H- |
| | pyrazole-1-carboxamide |
| 650 | 5-cyclopentyl-3-{2-hydroxy-4-[(3-methylbutanoyl)amino]phenyl}-N-propyl-1H- |
| | pyrazole-1-carboxamide |
| 651 | N-benzyl-5-cyclopentyl-3-{2-hydroxy-4-[(3-methylbutanoyl)amino]phenyl}-1H- |
| | pyrazole-1-carboxamide |
| 652 | 5-cyclopentyl-3-{2-hydroxy-4-[(3-methylbutanoyl)amino]phenyl}-N-(2- |
| | phenylethyl)-1H-pyrazole-1-carboxamide |
| 653 | 5-cyclopentyl-3-{2-hydroxy-4-[(3-methylbutanoyl)amino]phenyl}-N-(2- |
| | methylbenzyl)-1H-pyrazole-1-carboxamide |
| 654 | 5-cyclopentyl-3-{2-hydroxy-4-[(3-methylbutanoyl)amino]phenyl}-N-(3- |
| | methylbenzyl)-1H-pyrazole-1-carboxamide |
| 655 | 5-cyclopentyl-3-{2-hydroxy-4-[(3-methylbutanoyl)amino]phenyl}-N-(4- |
| | methylbenzyl)-1H-pyrazole-1-carboxamide |
| 656 | N-(2-chlorobenzyl)-5-cyclopentyl-3-{2-hydroxy-4-[(3- |
| | methylbutanoyl)amino]phenyl}-1H-pyrazole-1-carboxamide |
| 657 | 5-cyclopentyl-N-(4-fluorobenzyl)-3-{2-hydroxy-4-[(3- |
| | methylbutanoyl)amino]phenyl}-1H-pyrazole-1-carboxamide |
| 658 | 5-cyclopentyl-N-(3,4-dichlorobenzyl)-3-{2-hydroxy-4-[(3- |
| | methylbutanoyl)amino]phenyl}-1H-pyrazole-1-carboxamide |
| 659 | 5-cyclopentyl-3-{2-hydroxy-4-[(3-methylbutanoyl)amino]phenyl}-N-(4- |
| | methoxybenzyl)-1H-pyrazole-1-carboxamide |
| 660 | N-(sec-butyl)-5-cyclopentyl-3-{2-hydroxy-4-[(3-methylbutanoyl)amino]phenyl}- |
| | 1H-pyrazole-1-carboxamide |
| 661 | 5-cyclopentyl-N-(2-fluorobenzyl)-3-{2-hydroxy-4-[(3- |
| | methylbutanoyl)amino]phenyl}-1H-pyrazole-1-carboxamide |
| 662 | 5-cyclopentyl-N-(3-fluorobenzyl)-3-{2-hydroxy-4-[(3- |
| | methylbutanoyl)amino]phenyl}-1H-pyrazole-1-carboxamide |
| 663 | 5-cyclopentyl-N-cyclopropyl-3-{2-hydroxy-4-[(3- |
| | methylbutanoyl)amino]phenyl}-1H-pyrazole-1-carboxamide |
| 664 | 5-cyclopropyl-3-[2-hydroxy-4-(propionylamino)phenyl]-N-isopropyl-1H- |
| | pyrazole-1-carboxamide |
| 665 | 5-cyclopropyl-N-ethyl-3-[2-hydroxy-4-(propionylamino)phenyl]-1H-pyrazole-1- |
| | carboxamide |
| 666 | N-allyl-5-cyclopropyl-3-[2-hydroxy-4-(propionylamino)phenyl]-1H-pyrazole-1- |
| | carboxamide |
| 667 | 5-cyclopropyl-3-[2-hydroxy-4-(propionylamino)phenyl]-N-propyl-1H-pyrazole-1- |
| | carboxamide |
| 668 | N-benzyl-5-cyclopropyl-3-[2-hydroxy-4-(propionylamino)phenyl]-1H-pyrazole- |
| | 1-carboxamide |
| 669 | 5-cyclopropyl-3-[2-hydroxy-4-(propionylamino)phenyl]-N-(2-phenylethyl)-1H- |
| | pyrazole-1-carboxamide |
| 670 | 5-cyclopropyl-3-[2-hydroxy-4-(propionylamino)phenyl]-N-(2-methylbenzyl)-1H- |
| | pyrazole-1-carboxamide |
| 671 | 5-cyclopropyl-3-[2-hydroxy-4-(propionylamino)phenyl]-N-(3-methylbenzyl)-1H- |
| | pyrazole-1-carboxamide |
| 672 | N,5-dicyclopropyl-3-[2-hydroxy-4-(propionylamino)phenyl]-1H-pyrazole-1- |
| | carboxamide |
| 673 | N-(2-chlorobenzyl)-5-cyclopropyl-3-[2-hydroxy-4-(propionylamino)phenyl]-1H- |
| | pyrazole-1-carboxamide |
| 674 | 5-cyclopropyl-N-(4-fluorobenzyl)-3-[2-hydroxy-4-(propionylamino)phenyl]-1H- |
| | pyrazole-1-carboxamide |
| 675 | 5-cyclopropyl-N-(3,4-dichlorobenzyl)-3-[2-hydroxy-4-(propionylamino)phenyl]- |
| | 1H-pyrazole-1-carboxamide |
| 676 | 5-cyclopropyl-3-[2-hydroxy-4-(propionylamino)phenyl]-N-(4-methoxybenzyl)- |
| | 1H-pyrazole-1-carboxamide |
| 677 | N-(sec-butyl)-5-cyclopropyl-3-[2-hydroxy-4-(propionylamino)phenyl]-1H- |
| | pyrazole-1-carboxamide |
| 678 | 5-cyclopropyl-N-(2-fluorobenzyl)-3-[2-hydroxy-4-(propionylamino)phenyl]-1H- |
| | pyrazole-1-carboxamide |
| 679 | 5-cyclopropyl-N-(3-fluorobenzyl)-3-[2-hydroxy-4-(propionylamino)phenyl]-1H- |
| | pyrazole-1-carboxamide |
| 680 | N,5-dicyclopropyl-3-[2-hydroxy-4-(propionylamino)phenyl]-1H-pyrazole-1- |
| | carboxamide |
| 681 | 5-cyclobutyl-3-[2-hydroxy-4-(propionylamino)phenyl]-N-isopropyl-1H-pyrazole- |
| | 1-carboxamide |
| 682 | 5-cyclobutyl-N-ethyl-3-[2-hydroxy-4-(propionylamino)phenyl]-1H-pyrazole-1- |
| | carboxamide |
| 683 | N-allyl-5-cyclobutyl-3-[2-hydroxy-4-(propionylamino)phenyl]-1H-pyrazole-1- |
| | carboxamide |
| 684 | 5-cyclobutyl-3-[2-hydroxy-4-(propionylamino)phenyl]-N-propyl-1H-pyrazole-1- |
| | carboxamide |
| 685 | N-benzyl-5-cyclobutyl-3-[2-hydroxy-4-(propionylamino)phenyl]-1H-pyrazole-1- |
| | carboxamide |
| 686 | 5-cyclobutyl-3-[2-hydroxy-4-(propionylamino)phenyl]-N-(2-phenylethyl)-1H- |
| | pyrazole-1-carboxamide |
| 687 | 5-cyclobutyl-3-[2-hydroxy-4-(propionylamino)phenyl]-N-(2-methylbenzyl)-1H- |
| | pyrazole-1-carboxamide |
| 688 | 5-cyclobutyl-3-[2-hydroxy-4-(propionylamino)phenyl]-N-(3-methylbenzyl)-1H- |
| | pyrazole-1-carboxamide |
| 689 | 5-cyclobutyl-3-[2-hydroxy-4-(propionylamino)phenyl]-N-(4-methylbenzyl)-1H- |
| | pyrazole-1-carboxamide |
| 690 | N-(2-chlorobenzyl)-5-cyclobutyl-3-[2-hydroxy-4-(propionylamino)phenyl]-1H- |
| | pyrazole-1-carboxamide |
| 691 | 5-cyclobutyl-N-(4-fluorobenzyl)-3-[2-hydroxy-4-(propionylamino)phenyl]-1H- |
| | pyrazole-1-carboxamide |
| 692 | 5-cyclobutyl-N-(3,4-dichlorobenzyl)-3-[2-hydroxy-4-(propionylamino)phenyl]- |
| | 1H-pyrazole-1-carboxamide |
| 693 | 5-cyclobutyl-3-[2-hydroxy-4-(propionylamino)phenyl]-N-(4-methoxybenzyl)-1H- |
| | pyrazole-1-carboxamide |
| 694 | N-(sec-butyl)-5-cyclobutyl-3-[2-hydroxy-4-(propionylamino)phenyl]-1H- |
| | pyrazole-1-carboxamide |
| 695 | 5-cyclobutyl-N-(2-fluorobenzyl)-3-[2-hydroxy-4-(propionylamino)phenyl]-1H- |
| | pyrazole-1-carboxamide |
| 696 | 5-cyclobutyl-N-(3-fluorobenzyl)-3-[2-hydroxy-4-(propionylamino)phenyl]-1H- |
| | pyrazole-1-carboxamide |
| 697 | 5-cyclobutyl-N-cyclopropyl-3-[2-hydroxy-4-(propionylamino)phenyl]-1H- |
| | pyrazole-1-carboxamide |
| 698 | 5-cyclopentyl-3-[2-hydroxy-4-(propionylamino)phenyl]-N-isopropyl-1H- |
| | pyrazole-1-carboxamide |
| 699 | 5-cyclopentyl-N-ethyl-3-[2-hydroxy-4-(propionylamino)phenyl]-1H-pyrazole-1- |
| | carboxamide |
| 700 | N-allyl-5-cyclopentyl-3-[2-hydroxy-4-(propionylamino)phenyl]-1H-pyrazole-1- |
| | carboxamide |
| 701 | 5-cyclopentyl-3-[2-hydroxy-4-(propionylamino)phenyl]-N-propyl-1H-pyrazole-1- |
| | carboxamide |
| 702 | N-benzyl-5-cyclopentyl-3-[2-hydroxy-4-(propionylamino)phenyl]-1H-pyrazole-1- |
| | carboxamide |
| 703 | 5-cyclopentyl-3-[2-hydroxy-4-(propionylamino)phenyl]-N-(2-phenylethyl)-1H- |
| | pyrazole-1-carboxamide |
| 704 | 5-cyclopentyl-3-[2-hydroxy-4-(propionylamino)phenyl]-N-(2-methylbenzyl)-1H- |
| | pyrazole-1-carboxamide |
| 705 | 5-cyclopentyl-3-[2-hydroxy-4-(propionylamino)phenyl]-N-(3-methylbenzyl)-1H- |
| | pyrazole-1-carboxamide |
| 706 | 5-cyclopentyl-3-[2-hydroxy-4-(propionylamino)phenyl]-N-(4-methylbenzyl)-1H- |
| | pyrazole-1-carboxamide |
| 707 | N-(2-chlorobenzyl)-5-cyclopentyl-3-[2-hydroxy-4-(propionylamino)phenlyl]-1H- |
| | pyrazole-1-carboxamide |
| 708 | 5-cyclopentyl-N-(4-fluorobenzyl)-3-[2-hydroxy-4-(propionylamino)phenyl]-1H- |
| | pyrazole-1-carboxamide |
| 709 | 5-cyclopentyl-N-(3,4-dichlorobenzyl)-3-[2-hydroxy-4-(propionylamino)phenyl]- |
| | 1H-pyrazole-1-carboxamide |
| 710 | 5-cyclopentyl-3-[2-hydroxy-4-(propionylamino)phenyl]-N-(4-methoxybenzyl)- |
| | 1H-pyrazole-1-carboxamide |
| 711 | N-(sec-butyl)-5-cyclopentyl-3-[2-hydroxy-4-(propionylamino)phenyl]-1H- |
| | pyrazole-1-carboxamide |
| 712 | 5-cyclopentyl-N-(2-fluorobenzyl)-3-[2-hydroxy-4-(propionylamino)phenyl]-1H- |
| | pyrazole-1-carboxamide |
| 713 | 5-cyclopentyl-N-(3-fluorobenzyl)-3-[2-hydroxy-4-(propionylamino)phenyl]-1H- |
| | pyrazole-1-carboxamide |
| 714 | 5-cyclopentyl-N-cyclopropyl-3-[2-hydroxy-4-(propionylamino)phenyl]-1H- |
| | pyrazole-1-carboxamide |
| 715 | 5-cyclopropyl-3-{4-[(cyclopropylcarbonyl)amino]-2-hydroxyphenyl}-N- |
| | isopropyl-1H-pyrazole-1-carboxamide |
| 716 | 5-cyclopropyl-3-{4-[(cyclopropylcarbonyl)amino]-2-hydroxyphenyl}-N-ethyl- |
| | 1H-pyrazole-1-carboxamide |
| 717 | N-allyl-5-cyclopropyl-3-{4-[(cyclopropylcarbonyl)amino]-2-hydroxyphenyl}-1H- |
| | pyrazole-1-carboxamide |
| 718 | 5-cyclopropyl-3-{4-[(cyclopropylcarbonyl)amino]-2-hydroxyphenyl}-N-propyl- |
| | 1H-pyrazole-1-carboxamide |
| 719 | N-benzyl-5-cyclopropyl-3-{4-[(cyclopropylcarbonyl)amino]-2-hydroxyphenyl}- |
| | 1H-pyrazole-1-carboxamide |
| 720 | 5-cyclopropyl-3-{4-[(cyclopropylcarbonyl)amino]-2-hydroxyphenyl}-N-(2- |
| | phenylethyl)-1H-pyrazole-1-carboxamide |
| 721 | 5-cyclopropyl-3-{4-[(cyclopropylcarbonyl)amino]-2-hydroxyphenyl}-N-(2- |
| | methylbenzyl)-1H-pyrazole-1-carboxamide |
| 722 | 5-cyclopropyl-3-{4-[(cyclopropylcarbonyl)amino]-2-hydroxyphenyl}-N-(3- |
| | methylbenzyl)-1H-pyrazole-1-carboxamide |
| 723 | 5-cyclopropyl-3-{4-[(cyclopropylcarbonyl)amino]-2-hydroxyphenyl}-N-(4- |
| | methylbenzyl)-1H-pyrazole-1-carboxamide |
| 724 | N-(2-chlorobenzyl)-5-cyclopropyl-3-{4-[(cyclopropylcarbonyl)amino]-2- |
| | hydroxyphenyl}-1H-pyrazole-1-carboxamide |
| 725 | 5-cyclopropyl-3-{4-[(cyclopropylcarbonyl)amino]-2-hydroxyphenyl}-N-(4- |
| | fluorobenzyl)-1H-pyrazole-1-carboxamide |
| 726 | 5-cyclopropyl-3-{4-[(cyclopropylcarbonyl)amino]-2-hydroxyphenyl}-N-(3,4- |
| | dichlorobenzyl)-1H-pyrazole-1-carboxamide |
| 727 | 5-cyclopropyl-3-{4-[(cyclopropylcarbonyl)amino]-2-hydroxyphenyl}-N-(4- |
| | methoxybenzyl)-1H-pyrazole-1-carboxamide |
| 728 | N-(sec-butyl)-5-cyclopropyl-3-{4-[(cyclopropylcarbonyl)amino]-2- |
| | hydroxyphenyl}-1H-pyrazole-1-carboxamide |
| 729 | 5-cyclopropyl-3-{4-[(cyclopropylcarbonyl)amino]-2-hydroxyphenyl}-N-(2- |
| | fluorobenzyl)-1H-pyrazole-1-carboxamide |
| 730 | 5-cyclopropyl-3-{4-[(cyclopropylcarbonyl)amino]-2-hydroxyphenyl}-N-(3- |
| | fluorobenzyl)-1H-pyrazole-1-carboxamide |
| 731 | N,5-dicyclopropyl-3-{4-[(cyclopropylcarbonyl)amino]-2-hydroxyphenyl}-1H- |
| | pyrazole-1-carboxamide |
| 732 | 5-cyclobutyl-3-{4-[(cyclopropylcarbonyl)amino]-2-hydroxyphenyl}-N-isopropyl- |
| | 1H-pyrazole-1-carboxamide |
| 733 | 5-cyclobutyl-3-{4-[(cyclopropylcarbonyl)amino]-2-hydroxyphenyl}-N-ethyl-1H- |
| | pyrazole-1-carboxamide |
| 734 | N-allyl-5-cyclobutyl-3-{4-[(cyclopropylcarbonyl)amino]-2-hydroxyphenyl}-1H- |
| | pyrazole-1-carboxamide |
| 735 | 5-cyclobutyl-3-{4-[(cyclopropylcarbonyl)amino]-2-hydroxyphenyl}-N-propyl- |
| | 1H-pyrazole-1-carboxamide |
| 736 | N-benzyl-5-cyclobutyl-3-{4-[(cyclopropylcarbonyl)amino]-2-hydroxyphenyl}- |
| | 1H-pyrazole-1-carboxamide |
| 737 | 5-cyclobutyl-3-{4-[(cyclopropylcarbonyl)amino]-2-hydroxyphenyl}-N-(2- |
| | phenylethyl)-1H-pyrazole-1-carboxamide |
| 738 | 5-cyclobutyl-3-{4-[(cyclopropylcarbonyl)amino]-2-hydroxyphenyl}-N-(2- |
| | methylbenzyl)-1H-pyrazole-1-carboxamide |
| 739 | 5-cyclobutyl-3-{4-[(cyclopropylcarbonyl)amino]-2-hydroxyphenyl}-N-(3- |
| | methylbenzyl)-1H-pyrazole-1-carboxamide |
| 740 | 5-cyclobutyl-3-{4-[(cyclopropylcarbonyl)amino]-2-hydroxyphenyl}-N-(4- |
| | methylbenzyl)-1H-pyrazole-1-carboxamide |
| 741 | N-(2-chlorobenzyl)-5-cyclobutyl-3-{4-[(cyclopropylcarbonyl)amino]-2- |
| | hydroxyphenyl}-1H-pyrazole-1-carboxamide |
| 742 | 5-cyclobutyl-3-{4-[(cyclopropylcarbonyl)amino]-2-hydroxyphenyl}-N-(4- |
| | fluorobenzyl)-1H-pyrazole-1-carboxamide |
| 743 | 5-cyclobutyl-3-{4-[(cyclopropylcarbonyl)amino]-2-hydroxyphenyl}-N-(3,4- |
| | dichlorobenzyl)-1H-pyrazole-1-carboxamide |
| 744 | 5-cyclobutyl-3-{4-[(cyclopropylcarbonyl)amino]-2-hydroxyphenyl}-N-(4- |
| | methoxybenzyl)-1H-pyrazole-1-carboxamide |
| 745 | N-(sec-butyl)-5-cyclobutyl-3-{4-[(cyclopropylcarbonyl)amino]-2- |
| | hydroxyphenyl}-1H-pyrazole-1-carboxamide |
| 746 | 5-cyclobutyl-3-{4-[(cyclopropylcarbonyl)amino]-2-hydroxyphenyl}-N-(2- |
| | fluorobenzyl)-1H-pyrazole-1-carboxamide |
| 747 | 5-cyclobutyl-3-{4-[(cyclopropylcarbonyl)amino]-2-hydroxyphenyl}-N-(3- |
| | fluorobenzyl)-1H-pyrazole-1-carboxamide |
| 748 | 5-cyclobutyl-N-cyclopropyl-3-{4-[(cyclopropylcarbonyl)amino]-2- |
| | hydroxyphenyl}-1H-pyrazole-1-carboxamide |
| 749 | 5-cyclopentyl-3-{4-[(cyclopropylcarbonyl)amino]-2-hydroxyphenyl}-N- |
| | isopropyl-1H-pyrazole-1-carboxamide |
| 750 | 5-cyclopentyl-3-{4-[(cyclopropylcarbonyl)amino]-2-hydroxyphenyl}-N-ethyl- |
| | 1H-pyrazole-1-carboxamide |
| 751 | N-allyl-5-cyclopentyl-3-{4-[(cyclopropylcarbonyl)amino]-2-hydroxyphenyl}-1H- |
| | pyrazole-1-carboxamide |
| 752 | 5-cyclopentyl-3-{4-[(cyclopropylcarbonyl)amino]-2-hydroxyphenyl}-N-propyl- |
| | 1H-pyrazole-1-carboxamide |
| 753 | N-benzyl-5-cyclopentyl-3-{4-[(cyclopropylcarbonyl)amino]-2-hydroxyphenyl}- |
| | 1H-pyrazole-1-carboxamide |
| 754 | 5-cyclopentyl-3-{4-[(cyclopropylcarbonyl)amino]-2-hydroxyphenyl}-N-(2- |
| | phenylethyl)-1H-pyrazole-1-carboxamide |
| 755 | 5-cyclopentyl-3-{4-[(cyclopropylcarbonyl)amino]-2-hydroxyphenyl}-N-(2- |
| | methylbenzyl)-1H-pyrazole-1-carboxamide |
| 756 | 5-cyclopentyl-3-{4-[(cyclopropylcarbonyl)amino]-2-hydroxyphenyl}-N-(3- |
| | methylbenzyl)-1H-pyrazole-1-carboxamide |
| 757 | 5-cyclopentyl-3-{4-[(cyclopropylcarbonyl)amino]-2-hydroxyphenyl}-N-(4- |
| | methylbenzyl)-1H-pyrazole-1-carboxamide |
| 758 | N-(2-chlorobenzyl)-5-cyclopentyl-3-{4-[(cyclopropylcarbonyl)amino]-2- |
| | hydroxyphenyl}-1H-pyrazole-1-carboxamide |
| 759 | 5-cyclopentyl-3-{4-[(cyclopropylcarbonyl)amino]-2-hydroxyphenyl}-N-(4- |
| | fluorobenzyl)-1H-pyrazole-1-carboxamide |
| 760 | 5-cyclopentyl-3-{4-[(cyclopropylcarbonyl)amino]-2-hydroxyphenyl}-N-(3,4- |
| | dichlorobenzyl)-1H-pyrazole-1-carboxamide |
| 761 | 5-cyclopentyl-3-{4-[(cyclopropylcarbonyl)amino]-2-hydroxyphenyl}-N-(4- |
| | methoxybenzyl)-1H-pyrazole-1-carboxamide |
| 762 | N-(sec-butyl)-5-cyclopentyl-3-{4-[(cyclopropylcarbonyl)amino]-2- |
| | hydroxyphenyl}-1H-pyrazole-1-carboxamide |
| 763 | 5-cyclopentyl-3-{4-[(cyclopropylcarbonyl)amino]-2-hydroxyphenyl}-N-(2- |
| | fluorobenzyl)-1H-pyrazole-1-carboxamide |
| 764 | 5-cyclopentyl-3-{4-[(cyclopropylcarbonyl)amino]-2-hydroxyphenyl}-N-(3- |
| | fluorobenzyl)-1H-pyrazole-1-carboxamide |
| 765 | 5-cyclopentyl-N-cyclopropyl-3-{4-[(cyclopropylcarbonyl)amino]-2- |
| | hydroxyphenyl}-1H-pyrazole-1-carboxamide |
| 766 | 5-cyclopropyl-3-[4-(2-furoylamino)-2-hydroxyphenyl]-N-isopropyl-1H-pyrazole- |
| | 1-carboxamide |
| 767 | 5-cyclopropyl-N-ethyl-3-[4-(2-furoylamino)-2-hydroxyphenyl]-1H-pyrazole-1- |
| | carboxamide |
| 768 | N-allyl-5-cyclopropyl-3-[4-(2-furoylamino)-2-hydroxyphenyl]-1H-pyrazole-1- |
| | carboxamide |
| 769 | 5-cyclopropyl-3-[4-(2-furoylamino)-2-hydroxyphenyl]-N-propyl-1H-pyrazole-1- |
| | carboxamide |
| 770 | N-benzyl-5-cyclopropyl-3-[4-(2-furoylamino)-2-hydroxyphenyl]-1H-pyrazole-1- |
| | carboxamide |
| 771 | 5-cyclopropyl-3-[4-(2-furoylamino)-2-hydroxyphenyl]-N-(2-phenylethyl)-1H- |
| | pyrazole-1-carboxamide |
| 772 | 5-cyclopropyl-3-[4-(2-furoylamino)-2-hydroxyphenyl]-N-(2-methylbenzyl)-1H- |
| | pyrazole-1-carboxamide |
| 773 | 5-cyclopropyl-3-[4-(2-furoylamino)-2-hydroxyphenyl]-N-(3-methylbenzyl)-1H- |
| | pyrazole-1-carboxamide |
| 774 | 5-cyclopropyl-3-[4-(2-furoylamino)-2-hydroxyphenyl]-N-(4-methylbenzyl)-1H- |
| | pyrazole-1-carboxamide |
| 775 | N-(2-chlorobenzyl)-5-cyclopropyl-3-[4-(2-furoylamino)-2-hydroxyphenyl]-1H- |
| | pyrazole-1-carboxamide |
| 776 | 5-cyclopropyl-N-(4-fluorobenzyl)-3-[4-(2-furoylamino)-2-hydroxyphenyl]-1H- |
| | pyrazole-1-carboxamide |
| 777 | 5-cyclopropyl-N-(3,4-dichlorobenzyl)-3-[4-(2-furoylamino)-2-hydroxyphenyl]- |
| | 1H-pyrazole-1-carboxamide |
| 778 | 5-cyclopropyl-3-[4-(2-furoylamino)-2-hydroxyphenyl]-N-(4-methoxybenzyl)-1H- |
| | pyrazole-1-carboxamide |
| 779 | N-(sec-butyl)-5-cyclopropyl-3-[4-(2-furoylamino)-2-hydroxyphenyl]-1H- |
| | pyrazole-1-carboxamide |
| 780 | 5-cyclopropyl-N-(2-fluorobenzyl)-3-[4-(2-furoylamino)-2-hydroxyphenyl]-1H- |
| | pyrazole-1-carboxamide |
| 781 | 5-cyclopropyl-N-(3-fluorobenzyl)-3-[4-(2-furoylamino)-2-hydroxyphenyl]-1H- |
| | pyrazole-1-carboxamide |
| 782 | N,5-dicyclopropyl-3-[4-(2-furoylamino)-2-hydroxyphenyl]-1H-pyrazole-1- |
| | carboxamide |
| 783 | 5-cyclobutyl-3-[4-(2-furoylamino)-2-hydroxyphenyl]-N-isopropyl-1H-pyrazole-1- |
| | carboxamide |
| 784 | 5-cyclobutyl-N-ethyl-3-[4-(2-furoylamino)-2-hydroxyphenyl]-1H-pyrazole-1- |
| | carboxamide |
| 785 | N-allyl-5-cyclobutyl-3-[4-(2-furoylamino)-2-hydroxyphenyl]-1H-pyrazole-1- |
| | carboxamide |
| 786 | 5-cyclobutyl-3-[4-(2-furoylamino)-2-hydroxyphenyl]-N-propyl-1H-pyrazole-1- |
| | carboxamide |
| 787 | N-benzyl-5-cyclobutyl-3-[4-(2-furoylamino)-2-hydroxyphenyl]-1H-pyrazole-1- |
| | carboxamide |
| 788 | 5-cyclobutyl-3-[4-(2-furoylamino)-2-hydroxyphenyl]-N-(2-phenylethyl)-1H- |
| | pyrazole-1-carboxamide |
| 789 | 5-cyclobutyl-3-[4-(2-furoylamino)-2-hydroxyphenyl]-N-(2-methylbenzyl)-1H- |
| | pyrazole-1-carboxamide |
| 790 | 5-cyclobutyl-3-[4-(2-furoylamino)-2-hydroxyphenyl]-N-(3-methylbenzyl)-1H- |
| | pyrazole-1-carboxamide |
| 791 | 5-cyclobutyl-3-[4-(2-furoylamino)-2-hydroxyphenyl]-N-(4-methylbenzyl)-1H- |
| | pyrazole-1-carboxamide |
| 792 | N-(2-chlorobenzyl)-5-cyclobutyl-3-[4-(2-furoylamino)-2-hydroxyphenyl]-1H- |
| | pyrazole-1-carboxamide |
| 793 | 5-cyclobutyl-N-(4-fluorobenzyl)-3-[4-(2-furoylamino)-2-hydroxyphenyl]-1H- |
| | pyrazole-1-carboxamide |
| 794 | 5-cyclobutyl-N-(3,4-dichlorobenzyl)-3-[4-(2-furoylamino)-2-hydroxyphenyl]-1H- |
| | pyrazole-1-carboxamide |
| 795 | 5-cyclobutyl-3-[4-(2-furoylamino)-2-hydroxyphenyl]-N-(4-methoxybenzyl)-1H- |
| | pyrazole-1-carboxamide |
| 796 | N-(sec-butyl)-5-cyclobutyl-3-[4-(2-furoylamino)-2-hydroxyphenyl]-1H-pyrazole- |
| | 1-carboxamide |
| 797 | 5-cyclobutyl-N-(2-fluorobenzyl)-3-[4-(2-furoylamino)-2-hydroxyphenyl]-1H- |
| | pyrazole-1-carboxamide |
| 798 | 5-cyclobutyl-N-(3-fluorobenzyl)-3-[4-(2-furoylamino)-2-hydroxyphenyl]-1H- |
| | pyrazole-1-carboxamide |
| 799 | 5-cyclobutyl-N-cyclopropyl-3-[4-(2-furoylamino)-2-hydroxyphenyl]-1H- |
| | pyrazole-1-carboxamide |
| 800 | 5-cyclopentyl-3-[4-(2-furoylamino)-2-hydroxyphenyl]-N-isopropyl-1H-pyrazole- |
| | 1-carboxamide |
| 801 | 5-cyclopentyl-N-ethyl-3-[4-(2-furoylamino)-2-hydroxyphenyl]-1H-pyrazole-1- |
| | carboxamide |
| 802 | N-allyl-5-cyclopentyl-3-[4-(2-furoylamino)-2-hydroxyphenyl]-1H-pyrazole-1- |
| | carboxamide |
| 803 | 5-cyclopentyl-3-[4-(2-furoylamino)-2-hydroxyphenyl]-N-propyl-1H-pyrazole-1- |
| | carboxamide |
| 804 | 5-cyclopentyl-3-[4-(2-furoylamino)-2-hydroxyphenyl]-N-(2-methylbenzyl)-1H- |
| | pyrazole-1-carboxamide |
| 805 | 5-cyclopentyl-3-[4-(2-furoylamino)-2-hydroxyphenyl]-N-(2-phenylethyl)-1H- |
| | pyrazole-1-carboxamide |
| 806 | 5-cyclopentyl-3-[4-(2-furoylamino)-2-hydroxyphenyl]-N-(2-methylbenzyl)-1H- |
| | pyrazole-1-carboxamide |
| 807 | 5-cyclopentyl-3-[4-(2-furoylamino)-2-hydroxyphenyl]-N-(3-methylbenzyl)-1H- |
| | pyrazole-1-carboxamide |
| 808 | 5-cyclopentyl-3-[4-(2-furoylamino)-2-hydroxyphenyl]-N-(4-methylbenzyl)-1H- |
| | pyrazole-1-carboxamide |
| 809 | N-(2-chlorobenzyl)-5-cyclopentyl-3-[4-(2-furoylamino)-2-hydroxyphenyl]-1H- |
| | pyrazole-1-carboxamide |
| 810 | 5-cyclopentyl-N-(4-fluorobenzyl)-3-[4-(2-furoylamino)-2-hydroxyphenyl]-1H- |
| | pyrazole-1-carboxamide |
| 811 | 5-cyclopentyl-N-(3,4-dichlorobenzyl)-3-[4-(2-furoylamino)-2-hydroxyphenyl]- |
| | 1H-pyrazole-1-carboxamide |
| 812 | 5-cyclopentyl-3-[4-(2-furoylamino)-2-hydroxyphenyl]-N-(4-methoxybenzyl)-1H- |
| | pyrazole-1-carboxamide |
| 813 | N-(sec-butyl)-5-cyclopentyl-3-[4-(2-furoylamino)-2-hydroxyphenyl]-1H- |
| | pyrazole-1-carboxamide |
| 814 | 5-cyclopentyl-N-(2-fluorobenzyl)-3-[4-(2-furoylamino)-2-hydroxyphenyl]-1H- |
| | pyrazole-1-carboxamide |
| 815 | 5-cyclopentyl-N-(3-fluorobenzyl)-3-[4-(2-furoylamino)-2-hydroxyphenyl]-1H- |
| | pyrazole-1-carboxamide |
| 816 | 5-cyclopentyl-N-cyclopropyl-3-[4-(2-furoylamino)-2-hydroxyphenyl]-1H- |
| | pyrazole-1-carboxamide |
| 817 | 5-cyclopropyl-3-{2-hydroxy-4-[(thien-2-ylcarbonyl)amino]phenyl}-N-isopropyl- |
| | 1H-pyrazole-1-carboxamide |
| 818 | 5-cyclopropyl-N-ethyl-3-{2-hydroxy-4-[(thien-2-ylcarbonyl)amino]phenyl}-1H- |
| | pyrazole-1-carboxamide |
| 819 | N-allyl-5-cyclopropyl-3-{2-hydroxy-4-[(thien-2-ylcarbonyl)amino]phenyl}-1H- |
| | pyrazole-1-carboxamide |
| 820 | 5-cyclopropyl-3-{2-hydroxy-4-[(thien-2-ylcarbonyl)amino]phenyl}-N-propyl-1H- |
| | pyrazole-1-carboxamide |
| 821 | N-benzyl-5-cyclopropyl-3-{2-hydroxy-4-[(thien-2-ylcarbonyl)amino]phenyl}-1H- |
| | pyrazole-1-carboxamide |
| 822 | 5-cyclopropyl-3-{2-hydroxy-4-[(thien-2-ylcarbonyl)amino]phenyl}-N-(2- |
| | phenylethyl)-1H-pyrazole-1-carboxamide |
| 823 | 5-cyclopropyl-3-{2-hydroxy-4-[(thien-2-ylcarbonyl)amino]phenyl}-N-(2- |
| | methylbenzyl)-1H-pyrazole-1-carboxamide |
| 824 | 5-cyclopropyl-3-{2-hydroxy-4-[(thien-2-ylcarbonyl)amino]phenyl}-N-(3- |
| | methylbenzyl)-1H-pyrazole-1-carboxamide |
| 825 | 5-cyclopropyl-3-{2-hydroxy-4-[(thien-2-ylcarbonyl)amino]phenyl}-N-(4- |
| | methylbenzyl)-1H-pyrazole-1-carboxamide |
| 826 | N-(2-chlorobenzyl)-5-cyclopropyl-3-{2-hydroxy-4-[(thien-2- |
| | ylcarbonyl)amino]phenyl}-1H-pyrazole-1-carboxamide |
| 827 | 5-cyclopropyl-N-(4-fluorobenzyl)-3-{2-hydroxy-4-[(thien-2- |
| | ylcarbonyl)amino]phenyl}-1H-pyrazole-1-carboxamide |
| 828 | 5-cyclopropyl-N-(3,4-dichlorobenzyl)-3-{2-hydroxy-4-[(thien-2- |
| | ylcarbonyl)amino]phenyl}-1H-pyrazole-1-carboxamide |
| 829 | 5-cyclopropyl-3-{2-hydroxy-4-[(thien-2-ylcarbonyl)amino]phenyl}-N-(4- |
| | methoxybenzyl)-1H-pyrazole-1-carboxamide |
| 830 | N-(sec-butyl)-5-cyclopropyl-3-{2-hydroxy-4-[(thien-2- |
| | ylcarbonyl)amino]phenyl}-1H-pyrazole-1-carboxamide |
| 831 | 5-cyclopropyl-N-(2-fluorobenzyl)-3-{2-hydroxy-4-[(thien-2- |
| | ylcarbonyl)amino]phenyl}-1H-pyrazole-1-carboxamide |
| 832 | 5-cyclopropyl-N-(3-fluorobenzyl)-3-{2-hydroxy-4-[(thien-2- |
| | ylcarbonyl)amino]phenyl}-1H-pyrazole-1-carboxamide |
| 833 | N,5-dicyclopropyl-3-{2-hydroxy-4-[(thien-2-ylcarbonyl)amino]phenyl}-1H- |
| | pyrazole-1-carboxamide |
| 834 | 5-cyclobutyl-3-{2-hydroxy-4-[(thien-2-ylcarbonyl)amino]phenyl}-N-isopropyl- |
| | 1H-pyrazole-1-carboxamide |
| 835 | 5-cyclobutyl-N-ethyl-3-{2-hydroxy-4-[(thien-2-ylcarbonyl)amino]phenyl}-1H- |
| | pyrazole-1-carboxamide |
| 836 | N-allyl-5-cyclobutyl-3-{2-hydroxy-4-[(thien-2-ylcarbonyl)amino]phenyl}-1H- |
| | pyrazole-1-carboxamide |
| 837 | 5-cyclobutyl-3-{2-hydroxy-4-[(thien-2-ylcarbonyl)amino]phenyl}-N-propyl-1H- |
| | pyrazole-1-carboxamide |
| 838 | N-benzyl-5-cyclobutyl-3-{2-hydroxy-4-[(thien-2-ylcarbonyl)amino]phenyl}-1H- |
| | pyrazole-1-carboxamide |
| 839 | 5-cyclobutyl-3-{2-hydroxy-4-[(thien-2-ylcarbonyl)amino]phenyl}-N-(2- |
| | phenylethyl)-1H-pyrazole-1-carboxamide |
| 840 | 5-cyclobutyl-3-{2-hydroxy-4-[(thien-2-ylcarbonyl)amino]phenyl}-N-(2- |
| | methylbenzyl)-1H-pyrazole-1-carboxamide |
| 841 | 5-cyclobutyl-3-{2-hydroxy-4-[(thien-2-ylcarbonyl)amino]phenyl}-N-(3- |
| | methylbenzyl)-1H-pyrazole-1-carboxamide |
| 842 | 5-cyclobutyl-3-{2-hydroxy-4-[(thien-2-ylcarbonyl)amino]phenyl}-N-(4- |
| | methylbenzyl)-1H-pyrazole-1-carboxamide |
| 843 | N-(2-chlorobenzyl)-5-cyclobutyl-3-{2-hydroxy-4-[(thien-2- |
| | ylcarbonyl)amino]phenyl}-1H-pyrazole-1-carboxamide |
| 844 | 5-cyclobutyl-N-(4-fluorobenzyl)-3-{2-hydroxy-4-[(thien-2- |
| | ylcarbonyl)amino]phenyl}-1H-pyrazole-1-carboxamide |
| 845 | 5-cyclobutyl-N-(3,4-dichlorobenzyl)-3-{2-hydroxy-4-[(thien-2- |
| | ylcarbonyl)amino]phenyl}-1H-pyrazole-1-carboxamide |
| 846 | 5-cyclobutyl-3-{2-hydroxy-4-[(thien-2-ylcarbonyl)amino]phenyl}-N-(4- |
| | methoxybenzyl)-1H-pyrazole-1-carboxamide |
| 847 | N-(sec-butyl)-5-cyclobutyl-3-{2-hydroxy-4-[(thien-2-ylcarbonyl)amino]phenyl}- |
| | 1H-pyrazole-1-carboxamide |
| 848 | 5-cyclobutyl-N-(2-fluorobenzyl)-3-{2-hydroxy-4-[(thien-2- |
| | ylcarbonyl)amino]phenyl}-1H-pyrazole-1-carboxamide |
| 849 | 5-cyclobutyl-N-(3-fluorobenzyl)-3-{2-hydroxy-4-[(thien-2- |
| | ylcarbonyl)amino]phenyl}-1H-pyrazole-1-carboxamide |
| 850 | 5-cyclobutyl-N-cyclopropyl-3-{2-hydroxy-4-[(thien-2-ylcarbonyl)amino]phenyl}- |
| | 1H-pyrazole-1-carboxamide |
| 851 | 5-cyclopentyl-3-{2-hydroxy-4-[(thien-2-ylcarbonyl)amino]phenyl}-N-isopropyl- |
| | 1H-pyrazole-1-carboxamide |
| 852 | 5-cyclopentyl-N-ethyl-3-{2-hydroxy-4-[(thien-2-ylcarbonyl)amino]phenyl}-1H- |
| | pyrazole-1-carboxamide |
| 853 | N-allyl-5-cyclopentyl-3-{2-hydroxy-4-[(thien-2-ylcarbonyl)amino]phenyl}-1H- |
| | pyrazole-1-carboxamide |
| 854 | 5-cyclopentyl-3-{2-hydroxy-4-[(thien-2-ylcarbonyl)amino]phenyl}-N-propyl-1H- |
| | pyrazole-1-carboxamide |
| 855 | N-benzyl-5-cyclopentyl-3-{2-hydroxy-4-[(thien-2-ylcarbonyl)amino]phenyl}-1H- |
| | pyrazole-1-carboxamide |
| 856 | 5-cyclopentyl-3-{2-hydroxy-4-[(thien-2-ylcarbonyl)amino]phenyl}-N-(2- |
| | phenylethyl)-1H-pyrazole-1-carboxamide |
| 857 | 5-cyclopentyl-3-{2-hydroxy-4-[(thien-2-ylcarbonyl)amino]phenyl}-N-(2- |
| | methylbenzyl)-1H-pyrazole-1-carboxamide |
| 858 | 5-cyclopentyl-3-{2-hydroxy-4-[(thien-2-ylcarbonyl)amino]phenyl}-N-(3- |
| | methylbenzyl)-1H-pyrazole-1-carboxamide |
| 859 | 5-cyclopentyl-3-{2-hydroxy-4-[(thien-2-ylcarbonyl)amino]phenyl}-N-(4- |
| | methylbenzyl)-1H-pyrazole-1-carboxamide |
| 860 | N-(2-chlorobenzyl)-5-cyclopentyl-3-{2-hydroxy-4-[(thien-2- |
| | ylcarbonyl)amino]phenyl}-1H-pyrazole-1-carboxamide |
| 861 | 5-cyclopentyl-N-(4-fluorobenzyl)-3-{2-hydroxy-4-[(thien-2- |
| | ylcarbonyl)amino]phenyl}-1H-pyrazole-1-carboxamide |
| 862 | 5-cyclopentyl-N-(3,4-dichlorobenzyl)-3-{2-hydroxy-4-[(thien-2- |
| | ylcarbonyl)amino]phenyl}-1H-pyrazole-1-carboxamide |
| 863 | 5-cyclopentyl-3-{2-hydroxy-4-[(thien-2-ylcarbonyl)amino]phenyl}-N-(4- |
| | methoxybenzyl)-1H-pyrazole-1-carboxamide |
| 864 | N-(sec-butyl)-5-cyclopentyl-3-{2-hydroxy-4-[(thien-2-ylcarbonyl)amino]phenyl}- |
| | 1H-pyrazole-1-carboxamide |
| 865 | 5-cyclopentyl-N-(2-fluorobenzyl)-3-{2-hydroxy-4-[(thien-2- |
| | ylcarbonyl)amino]phenyl}-1H-pyrazole-1-carboxamide |
| 866 | 5-cyclopentyl-N-(3-fluorobenzyl)-3-{2-hydroxy-4-[(thien-2- |
| | ylcarbonyl)amino]phenyl}-1H-pyrazole-1-carboxamide |
| 867 | 5-cyclopentyl-N-cyclopropyl-3-{2-hydroxy-4-[(thien-2- |
| | ylcarbonyl)amino]phenyl}-1H-pyrazole-1-carboxamide |
| 868 | N-(4-{5-cyclopropyl-1-[(isopropylamino)carbonyl]-1H-pyrazol-3-yl}-3- |
| | hydroxyphenyl)quinoxaline-2-carboxamide |
| 869 | N-(4-{5-cyclopropyl-1-[(ethylamino)carbonyl]-1H-pyrazol-3-yl}-3- |
| | hydroxyphenyl)quinoxaline-2-carboxamide |
| 870 | N-(4-{1-[(allylamino)carbonyl]-5-cyclopropyl-1H-pyrazol-3-yl}-3- |
| | hydroxyphenyl)quinoxaline-2-carboxamide |
| 871 | N-(4-{5-cyclopropyl-1-[(propylamino)carbonyl]-1H-pyrazol-3-yl}-3- |
| | hydroxyphenyl)quinoxaline-2-carboxamide |
| 872 | N-(4-{1-[(benzylamino)carbonyl]-5-cyclopropyl-1H-pyrazol-3-yl}-3- |
| | hydroxyphenyl)quinoxaline-2-carboxamide |
| 873 | N-[4-(5-cyclopropyl-1-{[(2-phenylethyl)amino]carbonyl}-1H-pyrazol-3-yl)-3- |
| | hydroxyphenyl]quinoxaline-2-carboxamide |
| 874 | N-[4-(5-cyclopropyl-1-{[(2-methylbenzyl)amino]carbonyl}-1H-pyrazol-3-yl)-3- |
| | hydroxyphenyl]quinoxaline-2-carboxamide |
| 875 | N-[4-(5-cyclopropyl-1-{[(3-methylbenzyl)amino]carbonyl}-1H-pyrazol-3-yl)-3- |
| | hydroxyphenyl]quinoxaline-2-carboxamide |
| 876 | N-[4-(5-cyclopropyl-1-{[(4-methylbenzyl)amino]carbonyl}-1H-pyrazol-3-yl)-3- |
| | hydroxyphenyl]quinoxaline-2-carboxamide |
| 877 | N-[4-(1-{[(2-chlorobenzyl)amino]carbonyl}-5-cyclopropyl-1H-pyrazol-3-yl)-3- |
| | hydroxyphenyl]quinoxaline-2-carboxamide |
| 878 | N-[4-(5-cyclopropyl-1-{[(4-fluorobenzyl)amino]carbonyl}-1H-pyrazol-3-yl)-3- |
| | hydroxyphenyl]quinoxaline-2-carboxamide |
| 879 | N-[4-(5-cyclopropyl-1-{[(3,4-dichlorobenzyl)amino]carbonyl}-1H-pyrazol-3-yl)- |
| | 3-hydroxyphenyl]quinoxaline-2-carboxamide |
| 880 | N-[4-(5-cyclopropyl-1-{[(4-methoxybenzyl)amino]carbonyl}-1H-pyrazol-3-yl)-3- |
| | hydroxyphenyl]quinoxaline-2-carboxamide |
| 881 | N-(4-{1-[(sec-butylamino)carbonyl]-5-cyclopropyl-1H-pyrazol-3-yl}-3- |
| | hydroxyphenyl)quinoxaline-2-carboxamide |
| 882 | N-[4-(5-cyclopropyl-1-{[(2-fluorobenzyl)amino]carbonyl}-1H-pyrazol-3-yl)-3- |
| | hydroxyphenyl]quinoxaline-2-carboxamide |
| 883 | N-[4-(5-cyclopropyl-1-{[(3-fluorobenzyl)amino]carbonyl}-1H-pyrazol-3-yl)-3- |
| | hydroxyphenyl]quinoxaline-2-carboxamide |
| 884 | N-(4-{5-cyclopropyl-1-[(cyclopropylamino)carbonyl]-1H-pyrazol-3-yl}-3- |
| | hydroxyphenyl)quinoxaline-2-carboxamide |
| 885 | N-(4-{5-cyclobutyl-1-[(isopropylamino)carbonyl]-1H-pyrazol-3-yl}-3- |
| | hydroxyphenyl)quinoxaline-2-carboxamide |
| 886 | N-(4-{5-cyclobutyl-1-[(ethylamino)carbonyl]-1H-pyrazol-3-yl}-3- |
| | hydroxyphenyl)quinoxaline-2-carboxamide |
| 887 | N-(4-{1-[(allylamino)carbonyl]-5-cyclobutyl-1H-pyrazol-3-yl}-3- |
| | hydroxyphenyl)quinoxaline-2-carboxamide |
| 888 | N-(4-{5-cyclobutyl-1-[(propylamino)carbonyl]-1H-pyrazol-3-yl}-3- |
| | hydroxyphenyl)quinoxaline-2-carboxamide |
| 889 | N-(4-{1-[(benzylamino)carbonyl]-5-cyclobutyl-1H-pyrazol-3-yl}-3- |
| | hydroxyphenyl)quinoxaline-2-carboxamide |
| 890 | N-[4-(5-cyclobutyl-1-{[(2-phenylethyl)amino]carbonyl}-1H-pyrazol-3-yl)-3- |
| | hydroxyphenyl]quinoxaline-2-carboxamide |
| 891 | N-[4-(5-cyclobutyl-1-{[(2-methylbenzyl)amino]carbonyl}-1H-pyrazol-3-yl)-3- |
| | hydroxyphenyl]quinoxaline-2-carboxamide |
| 892 | N-[4-(5-cyclobutyl-1-{[(3-methylbenzyl)amino]carbonyl}-1H-pyrazol-3-yl)-3- |
| | hydroxyphenyl]quinoxaline-2-carboxamide |
| 893 | N-[4-(5-cyclobutyl-1-{[(4-methylbenzyl)amino]carbonyl}-1H-pyrazol-3-yl)-3- |
| | hydroxyphenyl]quinoxaline-2-carboxamide |
| 894 | N-[4-(1-{[(2-chlorobenzyl)amino]carbonyl}-5-cyclobutyl-1H-pyrazol-3-yl)-3- |
| | hydroxyphenyl]quinoxaline-2-carboxamide |
| 895 | N-[4-(5-cyclobutyl-1-{[(4-fluorobenzyl)amino]carbonyl}-1H-pyrazol-3-yl)-3- |
| | hydroxyphenyl]quinoxaline-2-carboxamide |
| 896 | N-[4-(5-cyclobutyl-1-{[(3,4-dichlorobenzyl)amino]carbonyl}-1H-pyrazol-3-yl)-3- |
| | hydroxyphenyl]quinoxaline-2-carboxamide |
| 897 | N-[4-(5-cyclobutyl-1-{[(4-methoxybenzyl)amino]carbonyl}-1H-pyrazol-3-yl)-3- |
| | hydroxyphenyl]quinoxaline-2-carboxamide |
| 898 | N-(4-{1-[(sec-butylamino)carbonyl]-5-cyclobutyl-1H-pyrazol-3-yl}-3- |
| | hydroxyphenyl)quinoxaline-2-carboxamide |
| 899 | N-[4-(5-cyclobutyl-1-{[(2-fluorobenzyl)amino]carbonyl}-1H-pyrazol-3-yl)-3- |
| | hydroxyphenyl]quinoxaline-2-carboxamide |
| 900 | N-[4-(5-cyclobutyl-1-{[(3-fluorobenzyl)amino]carbonyl}-1H-pyrazol-3-yl)-3- |
| | hydroxyphenyl]quinoxaline-2-carboxamide |
| 901 | N-(4-{5-cyclobutyl-1-[(cyclopropylamino)carbonyl]-1H-pyrazol-3-yl}-3- |
| | hydroxyphenyl)quinoxaline-2-carboxamide |
| 902 | N-(4-{5-cyclopentyl-1-[(isopropylamino)carbonyl]-1H-pyrazol-3-yl}-3- |
| | hydroxyphenyl)quinoxaline-2-carboxamide |
| 903 | N-(4-{5-cyclopentyl-1-[(ethylamino)carbonyl]-1H-pyrazol-3-yl}-3- |
| | hydroxyphenyl)quinoxaline-2-carboxamide |
| 904 | N-(4-{1-[(allylamino)carbonyl]-5-cyclopentyl-1H-pyrazol-3-yl}-3- |
| | hydroxyphenyl)quinoxaline-2-carboxamide |
| 905 | N-(4-{5-cyclopentyl-1-[(propylamino)carbonyl]-1H-pyrazol-3-yl}-3- |
| | hydroxyphenyl)quinoxaline-2-carboxamide |
| 906 | N-(4-{1-[(benzylamino)carbonyl]-5-cyclopentyl-1H-pyrazol-3-yl}-3- |
| | hydroxyphenyl)quinoxaline-2-carboxamide |
| 907 | N-[4-(5-cyclopentyl-1-{[(2-phenylethyl)amino]carbonyl}-1H-pyrazol-3-yl)-3- |
| | hydroxyphenyl]quinoxaline-2-carboxamide |
| 908 | N-[4-(5-cyclopentyl-1-{[(2-methylbenzyl)amino]carbonyl}-1H-pyrazol-3-yl)-3- |
| | hydroxyphenyl]quinoxaline-2-carboxamide |
| 909 | N-[4-(5-cyclopentyl-1-{[(3-methylbenzyl)amino]carbonyl}-1H-pyrazol-3-yl)-3- |
| | hydroxyphenyl]quinoxaline-2-carboxamide |
| 910 | N-[4-(5-cyclopentyl-1-{[(4-methylbenzyl)amino]carbonyl}-1H-pyrazol-3-yl)-3- |
| | hydroxyphenyl]quinoxaline-2-carboxamide |
| 911 | N-[4-(1-{[(2-chlorobenzyl)amino]carbonyl}-5-cyclopentyl-1H-pyrazol-3-yl)-3- |
| | hydroxyphenyl]quinoxaline-2-carboxamide |
| 912 | N-[4-(5-cyclopentyl-1-{[(4-fluorobenzyl)amino]carbonyl}-1H-pyrazol-3-yl)-3- |
| | hydroxyphenyl]quinoxaline-2-carboxamide |
| 913 | N-[4-(5-cyclopentyl-1-{[(3,4-dichlorobenzyl)amino]carbonyl}-1H-pyrazol-3-yl)- |
| | 3-hydroxyphenyl]quinoxaline-2-carboxamide |
| 914 | N-[4-(5-cyclopentyl-1-{[(4-methoxybenzyl)amino]carbonyl}-1H-pyrazol-3-yl)-3- |
| | hydroxyphenyl]quinoxaline-2-carboxamide |
| 915 | N-(4-{1-[(sec-butylamino)carbonyl]-5-cyclopentyl-1H-pyrazol-3-yl}-3- |
| | hydroxyphenyl)quinoxaline-2-carboxamide |
| 916 | N-[4-(5-cyclopentyl-1-{[(2-fluorobenzyl)amino]carbonyl}-1H-pyrazol-3-yl)-3- |
| | hydroxyphenyl]quinoxaline-2-carboxamide |
| 917 | N-[4-(5-cyclopentyl-1-{[(3-fluorobenzyl)amino]carbonyl}-1H-pyrazol-3-yl)-3- |
| | hydroxyphenyl]quinoxaline-2-carboxamide |
| 918 | N-(4-{5-cyclopentyl-1-[(cyclopropylamino)carbonyl]-1H-pyrazol-3-yl}-3- |
| | hydroxyphenyl)quinoxaline-2-carboxamide |
| 919 | N-(4-{5-cyclopropyl-1-[(isopropylamino)carbonyl]-1H-pyrazol-3-yl}-3- |
| | hydroxyphenyl)nicotinamide |
| 920 | N-(4-{5-cyclopropyl-1-[(ethylamino)carbonyl]-1H-pyrazol-3-yl}-3- |
| | hydroxyphenyl)nicotinamide |
| 921 | N-(4-{1-[(allylamino)carbonyl]-5-cyclopropyl-1H-pyrazol-3-yl}-3- |
| | hydroxyphenyl)nicotinamide |
| 922 | N-(4-{5-cyclopropyl-1-[(propylamino)carbonyl]-1H-pyrazol-3-yl}-3- |
| | hydroxyphenyl)nicotinamide |
| 923 | N-(4-{1-[(benzylamino)carbonyl]-5-cyclopropyl-1H-pyrazol-3-yl}-3- |
| | hydroxyphenyl)nicotinamide |
| 924 | N-[4-(5-cyclopropyl-1-{[(2-phenylethyl)amino]carbonyl}-1H-pyrazol-3-yl)-3- |
| | hydroxyphenyl]nicotinamide |
| 925 | N-[4-(5-cyclopropyl-1-{[(2-methylbenzyl)amino]carbonyl}-1H-pyrazol-3-yl)-3- |
| | hydroxyphenyl]nicotinamide |
| 926 | N-[4-(5-cyclopropyl-1-{[(3-methylbenzyl)amino]carbonyl}-1H-pyrazol-3-yl)-3- |
| | hydroxyphenyl]nicotinamide |
| 927 | N-[4-(5-cyclopropyl-1-{[(4-methylbenzyl)amino]carbonyl}-1H-pyrazol-3-yl)-3- |
| | hydroxyphenyl]nicotinamide |
| 928 | N-[4-(1-{[(2-chlorobenzyl)amino]carbonyl}-5-cyclopropyl-1H-pyrazol-3-yl)-3- |
| | hydroxyphenyl]nicotinamide |
| 929 | N-[4-(5-cyclopropyl-1-{[(4-fluorobenzyl)amino]carbonyl}-1H-pyrazol-3-yl)-3- |
| | hydroxyphenyl]nicotinamide |
| 930 | N-[4-(5-cyclopropyl-1-{[(3,4-dichlorobenzyl)amino]carbonyl}-1H-pyrazol-3-yl)- |
| | 3-hydroxyphenyl]nicotinamide |
| 931 | N-[4-(5-cyclopropyl-1-{[(4-methoxybenzyl)amino]carbonyl}-1H-pyrazol-3-yl)-3- |
| | hydroxyphenyl]nicotinamide |
| 932 | N-(4-{1-[(sec-butylamino)carbonyl]-5-cyclopropyl-1H-pyrazol-3-yl}-3- |
| | hydroxyphenyl)nicotinamide |
| 933 | N-[4-(5-cyclopropyl-1-{[(2-fluorobenzyl)amino]carbonyl}-1H-pyrazol-3-yl)-3- |
| | hydroxyphenyl]nicotinamide |
| 934 | N-[4-(5-cyclopropyl-1-{[(3-fluorobenzyl)amino]carbonyl}-1H-pyrazol-3-yl)-3- |
| | hydroxyphenyl]nicotinamide |
| 935 | N-(4-{5-cyclopropyl-1-[(cyclopropylamino)carbonyl]-1H-pyrazol-3-yl}-3- |
| | hydroxyphenyl)nicotinamide |
| 936 | N-(4-{5-cyclobutyl-1-[(isopropylamino)carbonyl]-1H-pyrazol-3-yl}-3- |
| | hydroxyphenyl)nicotinamide |
| 937 | N-(4-{5-cyclobutyl-1-[(ethylamino)carbonyl]-1H-pyrazol-3-yl}-3- |
| | hydroxyphenyl)nicotinamide |
| 938 | N-(4-{1-[(allylamino)carbonyl]-5-cyclobutyl-1H-pyrazol-3-yl}-3- |
| | hydroxyphenyl)nicotinamide |
| 939 | N-(4-{5-cyclobutyl-1-[(propylamino)carbonyl]-1H-pyrazol-3-yl}-3- |
| | hydroxyphenyl)nicotinamide |
| 940 | N-(4-{1-[(benzylamino)carbonyl]-5-cyclobutyl-1H-pyrazol-3-yl}-3- |
| | hydroxyphenyl)nicotinamide |
| 941 | N-[4-(5-cyclobutyl-1-{[(2-phenylethyl)amino]carbonyl}-1H-pyrazol-3-yl)-3- |
| | hydroxyphenyl]nicotinamide |
| 942 | N-[4-(5-cyclobutyl-1-{[(2-methylbenzyl)amino]carbonyl}-1H-pyrazol-3-yl)-3- |
| | hydroxyphenyl]nicotinamide |
| 943 | N-[4-(5-cyclobutyl-1-{[(3-methylbenzyl)amino]carbonyl}-1H-pyrazol-3-yl)-3- |
| | hydroxyphenyl]nicotinamide |
| 944 | N-[4-(5-cyclobutyl-1-{[(4-methylbenzyl)amino]carbonyl}-1H-pyrazol-3-yl)-3- |
| | hydroxyphenyl]nicotinamide |
| 945 | N-[4-(1-{[(2-chlorobenzyl)amino]carbonyl}-5-cyclobutyl-1H-pyrazol-3-yl)-3- |
| | hydroxyphenyl]nicotinamide |
| 946 | N-[4-(5-cyclobutyl-1-{[(4-fluorobenzyl)amino]carbonyl}-1H-pyrazol-3-yl)-3- |
| | hydroxyphenyl]nicotinamide |
| 947 | N-[4-(5-cyclobutyl-1-{[(3,4-dichlorobenzyl)amino]carbonyl}-1H-pyrazol-3-yl)-3- |
| | hydroxyphenyl]nicotinamide |
| 948 | N-[4-(5-cyclobutyl-1-{[(4-methoxybenzyl)amino]carbonyl}-1H-pyrazol-3-yl)-3- |
| | hydroxyphenyl]nicotinamide |
| 949 | N-(4-{1-[(sec-butylamino)carbonyl]-5-cyclobutyl-1H-pyrazol-3-yl}-3- |
| | hydroxyphenyl)nicotinamide |
| 950 | N-[4-(5-cyclobutyl-1-{[(2-fluorobenzyl)amino]carbonyl}-1H-pyrazol-3-yl)-3- |
| | hydroxyphenyl]nicotinamide |
| 951 | N-[4-(5-cyclobutyl-1-{[(3-fluorobenzyl)amino]carbonyl}-1H-pyrazol-3-yl)-3- |
| | hydroxyphenyl]nicotinamide |
| 952 | N-(4-{5-cyclobutyl-1-[(cyclopropylamino)carbonyl]-1H-pyrazol-3-yl}-3- |
| | hydroxyphenyl)nicotinamide |
| 953 | N-(4-{5-cyclopentyl-1-[(isopropylamino)carbonyl]-1H-pyrazol-3-yl}-3- |
| | hydroxyphenyl)nicotinamide |
| 954 | N-(4-{5-cyclopentyl-1-[(ethylamino)carbonyl]-1H-pyrazol-3-yl}-3- |
| | hydroxyphenyl)nicotinamide |
| 955 | N-(4-{1-[(allylamino)carbonyl]-5-cyclopentyl-1H-pyrazol-3-yl}-3- |
| | hydroxyphenyl)nicotinamide |
| 956 | N-(4-{5-cyclopentyl-1-[(propylamino)carbonyl]-1H-pyrazol-3-yl}-3- |
| | hydroxyphenyl)nicotinamide |
| 957 | N-(4-{1-[(benzylamino)carbonyl]-5-cyclopentyl-1H-pyrazol-3-yl}-3- |
| | hydroxyphenyl)nicotinamide |
| 958 | N-[4-(5-cyclopentyl-1-{[(2-phenylethyl)amino]carbonyl}-1H-pyrazol-3-yl)-3- |
| | hydroxyphenyl]nicotinamide |
| 959 | N-[4-(5-cyclopentyl-1-{[(2-methylbenzyl)amino]carbonyl}-1H-pyrazol-3-yl)-3- |
| | hydroxyphenyl]nicotinamide |
| 960 | N-[4-(5-cyclopentyl-1-{[(3-methylbenzyl)amino]carbonyl}-1H-pyrazol-3-yl)-3- |
| | hydroxyphenyl]nicotinamide |
| 961 | N-[4-(5-cyclopentyl-1-{[(4-methylbenzyl)amino]carbonyl}-1H-pyrazol-3-yl)-3- |
| | hydroxyphenyl]nicotinamide |
| 962 | N-[4-(1-{[(2-chlorobenzyl)amino]carbonyl}-5-cyclopentyl-1H-pyrazol-3-yl)-3- |
| | hydroxyphenyl]nicotinamide |
| 963 | N-[4-(5-cyclopentyl-1-{[(4-fluorobenzyl)amino]carbonyl}-1H-pyrazol-3-yl)-3- |
| | hydroxyphenyl]nicotinamide |
| 964 | N-[4-(5-cyclopentyl-1-{[(3,4-dichlorobenzyl)amino]carbonyl}-1H-pyrazol-3-yl)- |
| | 3-hydroxyphenyl]nicotinamide |
| 965 | N-(4-{1-[(sec-butylamino)carbonyl]-5-cyclopentyl-1H-pyrazol-3-yl}-3- |
| | hydroxyphenyl)nicotinamide |
| 966 | N-(4-{1-[(sec-butylamino)carbonyl]-5-cyclopentyl-1H-pyrazol-3-yl}-3- |
| | hydroxyphenyl)nicotinamide |
| 967 | N-[4-(5-cyclopentyl-1-{[(2-fluorobenzyl)amino]carbonyl}-1H-pyrazol-3-yl)-3- |
| | hydroxyphenyl]nicotinamide |
| 968 | N-[4-(5-cyclopentyl-1-{[(3-fluorobenzyl)amino]carbonyl}-1H-pyrazol-3-yl)-3- |
| | hydroxyphenyl]nicotinamide |
| 969 | N-(4-{5-cyclopentyl-1-[(cyclopropylamino)carbonyl]-1H-pyrazol-3-yl}-3- |
| | hydroxyphenyl)nicotinamide |
| 970 | N-(4-{5-cyclopropyl-1-[(isopropylamino)carbonyl]-1H-pyrazol-3-yl}-3- |
| | hydroxyphenyl)isonicotinamide |
| 971 | N-(4-{5-cyclopropyl-1-[(ethylamino)carbonyl]-1H-pyrazol-3-yl}-3- |
| | hydroxyphenyl)isonicotinamide |
| 972 | N-(4-{1-[(allylamino)carbonyl]-5-cyclopropyl-1H-pyrazol-3-yl}-3- |
| | hydroxyphenyl)isonicotinamide |
| 973 | N-(4-{5-cyclopropyl-1-[(propylamino)carbonyl]-1H-pyrazol-3-yl}-3- |
| | hydroxyphenyl)isonicotinamide |
| 974 | N-(4-{1-[(benzylamino)carbonyl]-5-cyclopropyl-1H-pyrazol-3-yl}-3- |
| | hydroxyphenyl)isonicotinamide |
| 975 | N-[4-(5-cyclopropyl-1-{[(2-phenylethyl)amino]carbonyl}-1H-pyrazol-3-yl)-3- |
| | hydroxyphenyl]isonicotinamide |
| 976 | N-[4-(5-cyclopropyl-1-{[(2-methylbenzyl)amino]carbonyl}-1H-pyrazol-3-yl)-3- |
| | hydroxyphenyl]isonicotinamide |
| 977 | N-[4-(5-cyclopropyl-1-{[(3-methylbenzyl)amino]carbonyl}-1H-pyrazol-3-yl)-3- |
| | hydroxyphenyl]isonicotinamide |
| 978 | N-[4-(5-cyclopropyl-1-{[(4-methylbenzyl)amino]carbonyl}-1H-pyrazol-3-yl)-3- |
| | hydroxyphenyl]isonicotinamide |
| 979 | N-[4-(1-{[(2-chlorobenzyl)amino]carbonyl}-5-cyclopropyl-1H-pyrazol-3-yl)-3- |
| | hydroxyphenyl]isonicotinamide |
| 980 | N-[4-(5-cyclopropyl-1-{[(4-fluorobenzyl)amino]carbonyl}-1H-pyrazol-3-yl)-3- |
| | hydroxyphenyl]isonicotinamide |
| 981 | N-[4-(5-cyclopropyl-1-{[(3,4-dichlorobenzyl)amino]carbonyl}-1H-pyrazol-3-yl)- |
| | 3-hydroxyphenyl]isonicotinamide |
| 982 | N-[4-(5-cyclopropyl-1-{[(4-methoxybenzyl)amino]carbonyl}-1H-pyrazol-3-yl)-3- |
| | hydroxyphenyl]isonicotinamide |
| 983 | N-(4-{1-[(sec-butylamino)carbonyl]-5-cyclopropyl-1H-pyrazol-3-yl}-3- |
| | hydroxyphenyl)isonicotinamide |
| 984 | N-[4-(5-cyclopropyl-1-{[(2-fluorobenzyl)amino]carbonyl}-1H-pyrazol-3-yl)-3- |
| | hydroxyphenyl]isonicotinamide |
| 985 | N-[4-(5-cyclopropyl-1-{[(3-fluorobenzyl)amino]carbonyl}-1H-pyrazol-3-yl)-3- |
| | hydroxyphenyl]isonicotinamide |
| 986 | N-(4-{5-cyclopropyl-1-[(cyclopropylamino)carbonyl]-1H-pyrazol-3-yl}-3- |
| | hydroxyphenyl)isonicotinamide |
| 987 | N-(4-{5-cyclobutyl-1-[(isopropylamino)carbonyl]-1H-pyrazol-3-yl}-3- |
| | hydroxyphenyl)isonicotinamide |
| 988 | N-(4-{5-cyclobutyl-1-[(ethylamino)carbonyl]-1H-pyrazol-3-yl}-3- |
| | hydroxyphenyl)isonicotinamide |
| 989 | N-(4-{1-[(allylamino)carbonyl]-5-cyclobutyl-1H-pyrazol-3-yl}-3- |
| | hydroxyphenyl)isonicotinamide |
| 990 | N-(4-{5-cyclobutyl-1-[(propylamino)carbonyl]-1H-pyrazol-3-yl}-3- |
| | hydroxyphenyl)isonicotinamide |
| 991 | N-(4-{1-[(benzylamino)carbonyl]-5-cyclobutyl-1H-pyrazol-3-yl}-3- |
| | hydroxyphenyl)isonicotinamide |
| 992 | N-[4-(5-cyclobutyl-1-{[(2-methylbenzyl)amino]carbonyl}-1H-pyrazol-3-yl)-3- |
| | hydroxyphenyl]isonicotinamide |
| 993 | N-[4-(5-cyclobutyl-1-{[(2-methylbenzyl)amino]carbonyl}-1H-pyrazol-3-yl)-3- |
| | hydroxyphenyl]isonicotinamide |
| 994 | N-[4-(5-cyclobutyl-1-{[(3-methylbenzyl)amino]carbonyl}-1H-pyrazol-3-yl)-3- |
| | hydroxyphenyl]isonicotinamide |
| 995 | N-[4-(5-cyclobutyl-1-{[(4-methylbenzyl)amino]carbonyl}-1H-pyrazol-3-yl)-3- |
| | hydroxyphenyl]isonicotinamide |
| 996 | N-[4-(1-{[(2-chlorobenzyl)amino]carbonyl}-5-cyclobutyl-1H-pyrazol-3-yl)-3- |
| | hydroxyphenyl]isonicotinamide |
| 997 | N-[4-(5-cyclobutyl-1-{[(4-fluorobenzyl)amino]carbonyl}-1H-pyrazol-3-yl)-3- |
| | hydroxyphenyl]isonicotinamide |
| 998 | N-[4-(5-cyclobutyl-1-{[(3,4-dichlorobenzyl)amino]carbonyl}-1H-pyrazol-3-yl)-3- |
| | hydroxyphenyl]isonicotinamide |
| 999 | N-[4-(5-cyclobutyl-1-{[(4-methoxybenzyl)amino]carbonyl}-1H-pyrazol-3-yl)-3- |
| | hydroxyphenyl]isonicotinamide |
| 1000 | N-(4-{1-[(sec-butylamino)carbonyl]-5-cyclobutyl-1H-pyrazol-3-yl}-3- |
| | hydroxyphenyl)isonicotinamide |
| 1001 | N-[4-(5-cyclobutyl-1-{[(2-fluorobenzyl)amino]carbonyl}-1H-pyrazol-3-yl)-3- |
| | hydroxyphenyl]isonicotinamide |
| 1002 | N-[4-(5-cyclobutyl-1-{[(3-fluorobenzyl)amino]carbonyl}-1H-pyrazol-3-yl)-3- |
| | hydroxyphenyl]isonicotinamide |
| 1003 | N-(4-{5-cyclobutyl-1-[(cyclopropylamino)carbonyl]-1H-pyrazol-3-yl}-3- |
| | hydroxyphenyl)isonicotinamide |
| 1004 | N-(4-{5-cyclopentyl-1-[(isopropylamino)carbonyl]-1H-pyrazol-3-yl}-3- |
| | hydroxyphenyl)isonicotinamide |
| 1005 | N-(4-{5-cyclopentyl-1-[(ethylamino)carbonyl]-1H-pyrazol-3-yl}-3- |
| | hydroxyphenyl)isonicotinamide |
| 1006 | N-(4-{1-[(allylamino)carbonyl]-5-cyclopentyl-1H-pyrazol-3-yl}-3- |
| | hydroxyphenyl)isonicotinamide |
| 1007 | N-(4-{5-cyclopentyl-1-[(propylamino)carbonyl]-1H-pyrazol-3-yl}-3- |
| | hydroxyphenyl)isonicotinamide |
| 1008 | N-(4-{1-[(benzylamino)carbonyl]-5-cyclopentyl-1H-pyrazol-3-yl}-3- |
| | hydroxyphenyl)isonicotinamide |
| 1009 | N-[4-(5-cyclopentyl-1-{[(2-phenylethyl)amino]carbonyl}-1H-pyrazol-3-yl)-3- |
| | hydroxyphenyl]isonicotinamide |
| 1010 | N-[4-(5-cyclopentyl-1-{[(2-methylbenzyl)amino]carbonyl}-1H-pyrazol-3-yl)-3- |
| | hydroxyphenyl]isonicotinamide |
| 1011 | N-[4-(5-cyclopentyl-1-{[(3-methylbenzyl)amino]carbonyl}-1H-pyrazol-3-yl)-3- |
| | hydroxyphenyl]isonicotinamide |
| 1012 | N-[4-(5-cyclopentyl-1-{[(4-methylbenzyl)amino]carbonyl}-1H-pyrazol-3-yl)-3- |
| | hydroxyphenyl]isonicotinamide |
| 1013 | N-[4-(1-{[(2-chlorobenzyl)amino]carbonyl}-5-cyclopentyl-1H-pyrazol-3-yl)-3- |
| | hydroxyphenyl]isonicotinamide |
| 1014 | N-[4-(5-cyclopentyl-1-{[(4-fluorobenzyl)amino]carbonyl}-1H-pyrazol-3-yl)-3- |
| | hydroxyphenyl]isonicotinamide |
| 1015 | N-[4-(5-cyclopentyl-1-{[(3,4-dichlorobenzyl)amino]carbonyl}-1H-pyrazol-3-yl)- |
| | 3-hydroxyphenyl]isonicotinamide |
| 1016 | N-[4-(5-cyclopentyl-1-{[(4-methoxybenzyl)amino]carbonyl}-1H-pyrazol-3-yl)-3- |
| | hydroxyphenyl]isonicotinamide |
| 1017 | N-(4-{1-[(sec-butylamino)carbonyl]-5-cyclopentyl-1H-pyrazol-3-yl}-3- |
| | hydroxyphenyl)isonicotinamide |
| 1018 | N-[4-(5-cyclopentyl-1-{[(2-fluorobenzyl)amino]carbonyl}-1H-pyrazol-3-yl)-3- |
| | hydroxyphenyl]isonicotinamide |
| 1019 | N-[4-(5-cyclopentyl-1-{[(3-fluorobenzyl)amino]carbonyl}-1H-pyrazol-3-yl)-3- |
| | hydroxyphenyl]isonicotinamide |
| 1020 | N-(4-{5-cyclopentyl-1-[(cyclopropylamino)carbonyl]-1H-pyrazol-3-yl}-3- |
| | hydroxyphenyl)isonicotinamide |
| 1021 | 3-{4-[(1,3-benzodioxol-5-ylcarbonyl)amino]-2-hydroxyphenyl}-5-cyclopropyl-N- |
| | isopropyl-1H-pyrazole-1-carboxamide |
| 1022 | 3-{4-[(1,3-benzodioxol-5-ylcarbonyl)amino]-2-hydroxyphenyl}-5-cyclopropyl-N- |
| | ethyl-1H-pyrazole-1-carboxamide |
| 1023 | N-allyl-3-{4-[(1,3-benzodioxol-5-ylcarbonyl)amino]-2-hydroxyphenyl}-5- |
| | cyclopropyl-1H-pyrazole-1-carboxamide |
| 1024 | 3-{4-[(1,3-benzodioxol-5-ylcarbonyl)amino]-2-hydroxyphenyl}-5-cyclopropyl-N- |
| | propyl-1H-pyrazole-1-carboxamide |
| 1025 | 3-{4-[(1,3-benzodioxol-5-ylcarbonyl)amino]-2-hydroxyphenyl}-N-benzyl-5- |
| | cyclopropyl-1H-pyrazole-1-carboxamide |
| 1026 | 3-{4-[(1,3-benzodioxol-5-ylcarbonyl)amino]-2-hydroxyphenyl}-5-cyclopropyl-N- |
| | (2-phenylethyl)-1H-pyrazole-1-carboxamide |
| 1027 | 3-{4-[(1,3-benzodioxol-5-ylcarbonyl)amino]-2-hydroxyphenyl}-5-cyclopropyl-N- |
| | (2-methylbenzyl)-1H-pyrazole-1-carboxamide |
| 1028 | 3-{4-[(1,3-benzodioxol-5-ylcarbonyl)amino]-2-hydroxyphenyl}-5-cyclopropyl-N- |
| | (3-methylbenzyl)-1H-pyrazole-1-carboxamide |
| 1029 | 3-{4-[(1,3-benzodioxol-5-ylcarbonyl)amino]-2-hydroxyphenyl}-5-cyclopropyl-N- |
| | (4-methylbenzyl)-1H-pyrazole-1-carboxamide |
| 1030 | 3-{4-[(1,3-benzodioxol-5-ylcarbonyl)amino]-2-hydroxyphenyl}-N-(2- |
| | chlorobenzyl)-5-cyclopropyl-1H-pyrazole-1-carboxamide |
| 1031 | 3-{4-[(1,3-benzodioxol-5-ylcarbonyl)amino]-2-hydroxyphenyl}-5-cyclopropyl-N- |
| | (4-fluorobenzyl)-1H-pyrazole-1-carboxamide |
| 1032 | 3-{4-[(1,3-benzodioxol-5-ylcarbonyl)amino]-2-hydroxyphenyl}-5-cyclopropyl-N- |
| | (3,4-dichlorobenzyl)-1H-pyrazole-1-carboxamide |
| 1033 | 3-{4-[(1,3-benzodioxol-5-ylcarbonyl)amino]-2-hydroxyphenyl}-5-cyclopropyl-N- |
| | (4-methoxybenzyl)-1H-pyrazole-1-carboxamide |
| 1034 | 3-{4-[(1,3-benzodioxol-5-ylcarbonyl)amino]-2-hydroxyphenyl}-N-(sec-butyl)-5- |
| | cyclopropyl-1H-pyrazole-1-carboxamide |
| 1035 | 3-{4-[(1,3-benzodioxol-5-ylcarbonyl)amino]-2-hydroxyphenyl}-5-cyclopropyl-N- |
| | (2-fluorobenzyl)-1H-pyrazole-1-carboxamide |
| 1036 | 3-{4-[(1,3-benzodioxol-5-ylcarbonyl)amino]-2-hydroxyphenyl}-5-cyclopropyl-N- |
| | (3-fluorobenzyl)-1H-pyrazole-1-carboxamide |
| 1037 | 3-{4-[(1,3-benzodioxol-5-ylcarbonyl)amino]-2-hydroxyphenyl}-N,5- |
| | dicyclopropyl-1H-pyrazole-1-carboxamide |
| 1038 | 3-{4-[(1,3-benzodioxol-5-ylcarbonyl)amino]-2-hydroxyphenyl}-5-cyclobutyl-N- |
| | isopropyl-1H-pyrazole-1-carboxamide |
| 1039 | 3-{4-[(1,3-benzodioxol-5-ylcarbonyl)amino]-2-hydroxyphenyl}-5-cyclobutyl-N- |
| | ethyl-1H-pyrazole-1-carboxamide |
| 1040 | N-allyl-3-{4-[(1,3-benzodioxol-5-ylcarbonyl)amino]-2-hydroxyphenyl}-5- |
| | cyclobutyl-1H-pyrazole-1-carboxamide |
| 1041 | 3-{4-[(1,3-benzodioxol-5-ylcarbonyl)amino]-2-hydroxyphenyl}-5-cyclobutyl-N- |
| | propyl-1H-pyrazole-1-carboxamide |
| 1042 | 3-{4-[(1,3-benzodioxol-5-ylcarbonyl)amino]-2-hydroxyphenyl}-N-benzyl-5- |
| | cyclobutyl-1H-pyrazole-1-carboxamide |
| 1043 | 3-{4-[(1,3-benzodioxol-5-ylcarbonyl)amino]-2-hydroxyphenyl}-5-cyclobutyl-N- |
| | (2-phenylethyl)-1H-pyrazole-1-carboxamide |
| 1044 | 3-{4-[(1,3-benzodioxol-5-ylcarbonyl)amino]-2-hydroxyphenyl}-5-cyclobutyl-N- |
| | (2-methylbenzyl)-1H-pyrazole-1-carboxamide |
| 1045 | 3-{4-[(1,3-benzodioxol-5-ylcarbonyl)amino]-2-hydroxyphenyl}-5-cyclobutyl-N- |
| | (3-methylbenzyl)-1H-pyrazole-1-carboxamide |
| 1046 | 3-{4-[(1,3-benzodioxol-5-ylcarbonyl)amino]-2-hydroxyphenyl}-5-cyclobutyl-N- |
| | (4-methylbenzyl)-1H-pyrazole-1-carboxamide |
| 1047 | 3-{4-[(1,3-benzodioxol-5-ylcarbonyl)amino]-2-hydroxyphenyl}-N-(2- |
| | chlorobenzyl)-5-cyclobutyl-1H-pyrazole-1-carboxamide |
| 1048 | 3-{4-[(1,3-benzodioxol-5-ylcarbonyl)amino]-2-hydroxyphenyl}-5-cyclobutyl-N- |
| | (4-fluorobenzyl)-1H-pyrazole-1-carboxamide |
| 1049 | 3-{4-[(1,3-benzodioxol-5-ylcarbonyl)amino]-2-hydroxyphenyl}-5-cyclobutyl-N- |
| | (3,4-dichlorobenzyl)-1H-pyrazole-1-carboxamide |
| 1050 | 3-{4-[(1,3-benzodioxol-5-ylcarbonyl)amino]-2-hydroxyphenyl}-5-cyclobutyl-N- |
| | (4-methoxybenzyl)-1H-pyrazole-1-carboxamide |
| 1051 | 3-{4-[(1,3-benzodioxol-5-ylcarbonyl)amino]-2-hydroxyphenyl}-N-(sec-butyl)-5- |
| | cyclobutyl-1H-pyrazole-1-carboxamide |
| 1052 | 3-{4-[(1,3-benzodioxol-5-ylcarbonyl)amino]-2-hydroxyphenyl}-5-cyclobutyl-N- |
| | (2-fluorobenzyl)-1H-pyrazole-1-carboxamide |
| 1053 | 3-{4-[(1,3-benzodioxol-5-ylcarbonyl)amino]-2-hydroxyphenyl}-5-cyclobutyl-N- |
| | (3-fluorobenzyl)-1H-pyrazole-1-carboxamide |
| 1054 | 3-{4-[(1,3-benzodioxol-5-ylcarbonyl)amino]-2-hydroxyphenyl}-5-cyclobutyl-N- |
| | cyclopropyl-1H-pyrazole-1-carboxamide |
| 1055 | 3-{4-[(1,3-benzodioxol-5-ylcarbonyl)amino]-2-hydroxyphenyl}-5-cyclopentyl-N- |
| | isopropyl-1H-pyrazole-1-carboxamide |
| 1056 | 3-{4-[(1,3-benzodioxol-5-ylcarbonyl)amino]-2-hydroxyphenyl}-5-cyclopentyl-N- |
| | ethyl-1H-pyrazole-1-carboxamide |
| 1057 | N-allyl-3-{4-[(1,3-benzodioxol-5-ylcarbonyl)amino]-2-hydroxyphenyl}-5- |
| | cyclopentyl-1H-pyrazole-1-carboxamide |
| 1058 | 3-{4-[(1,3-benzodioxol-5-ylcarbonyl)amino]-2-hydroxyphenyl}-5-cyclopentyl-N- |
| | propyl-1H-pyrazole-1-carboxamide |
| 1059 | 3-{4-[(1,3-benzodioxol-5-ylcarbonyl)amino]-2-hydroxyphenyl}-N-benzyl-5- |
| | cyclopentyl-1H-pyrazole-1-carboxamide |
| 1060 | 3-{4-[(1,3-benzodioxol-5-ylcarbonyl)amino]-2-hydroxyphenyl}-5-cyclopentyl-N- |
| | (2-phenylethyl)-1H-pyrazole-1-carboxamide |
| 1061 | 3-{4-[(1,3-benzodioxol-5-ylcarbonyl)amino]-2-hydroxyphenyl}-5-cyclopentyl-N- |
| | (2-methylbenzyl)-1H-pyrazole-1-carboxamide |
| 1062 | 3-{4-[(1,3-benzodioxol-5-ylcarbonyl)amino]-2-hydroxyphenyl}-5-cyclopentyl-N- |
| | (3-methylbenzyl)-1H-pyrazole-1-carboxamide |
| 1063 | 3-{4-[(1,3-benzodioxol-5-ylcarbonyl)amino]-2-hydroxyphenyl}-5-cyclopentyl-N- |
| | (4-methylbenzyl)-1H-pyrazole-1-carboxamide |
| 1064 | 3-{4-[(1,3-benzodioxol-5-ylcarbonyl)amino]-2-hydroxyphenyl}-N-(2- |
| | chlorobenzyl)-5-cyclopentyl-1H-pyrazole-1-carboxamide |
| 1065 | 3-{4-[(1,3-benzodioxol-5-ylcarbonyl)amino]-2-hydroxyphenyl}-5-cyclopentyl-N- |
| | (4-fluorobenzyl)-1H-pyrazole-1-carboxamide |
| 1066 | 3-{4-[(1,3-benzodioxol-5-ylcarbonyl)amino]-2-hydroxyphenyl}-5-cyclopentyl-N- |
| | (3,4-dichlorobenzyl)-1H-pyrazole-1-carboxamide |
| 1067 | 3-{4-[(1,3-benzodioxol-5-ylcarbonyl)amino]-2-hydroxyphenyl}-5-cyclopentyl-N- |
| | (4-methoxybenzyl)-1H-pyrazole-1-carboxamide |
| 1068 | 3-{4-[(1,3-benzodioxol-5-ylcarbonyl)amino]-2-hydroxyphenyl}-N-(sec-butyl)-5- |
| | cyclopentyl-1H-pyrazole-1-carboxamide |
| 1069 | 3-{4-[(1,3-benzodioxol-5-ylcarbonyl)amino]-2-hydroxyphenyl}-5-cyclopentyl-N- |
| | (2-fluorobenzyl)-1H-pyrazole-1-carboxamide |
| 1070 | 3-{4-[(1,3-benzodioxol-5-ylcarbonyl)amino]-2-hydroxyphenyl}-5-cyclopentyl-N- |
| | (3-fluorobenzyl)-1H-pyrazole-1-carboxamide |
| 1071 | 3-{4-[(1,3-benzodioxol-5-ylcarbonyl)amino]-2-hydroxyphenyl}-5-cyclopentyl-N- |
| | cyclopropyl-1H-pyrazole-1-carboxamide |
| 1072 | 5-cyclopropyl-3-{4-[(2-ethylbutanoyl)amino]-2-hydroxyphenyl}-N-isopropyl-1H- |
| | pyrazole-1-carboxamide |
| 1073 | 5-cyclopropyl-N-ethyl-3-{4-[(2-ethylbutanoyl)amino]-2-hydroxyphenyl}-1H- |
| | pyrazole-1-carboxamide |
| 1074 | N-allyl-5-cyclopropyl-3-{4-[(2-ethylbutanoyl)amino]-2-hydroxyphenyl}-1H- |
| | pyrazole-1-carboxamide |
| 1075 | 5-cyclopropyl-3-{4-[(2-ethylbutanoyl)amino]-2-hydroxyphenyl}-N-propyl-1H- |
| | pyrazole-1-carboxamide |
| 1076 | N-benzyl-5-cyclopropyl-3-{4-[(2-ethylbutanoyl)amino]-2-hydroxyphenyl}-1H- |
| | pyrazole-1-carboxamide |
| 1077 | 5-cyclopropyl-3-{4-[(2-ethylbutanoyl)amino]-2-hydroxyphenyl}-N-(2- |
| | phenylethyl)-1H-pyrazole-1-carboxamide |
| 1078 | 5-cyclopropyl-3-{4-[(2-ethylbutanoyl)amino]-2-hydroxyphenyl}-N-(2- |
| | methylbenzyl)-1H-pyrazole-1-carboxamide |
| 1079 | 5-cyclopropyl-3-{4-[(2-ethylbutanoyl)amino]-2-hydroxyphenyl}-N-(3- |
| | methylbenzyl)-1H-pyrazole-1-carboxamide |
| 1080 | 5-cyclopropyl-3-{4-[(2-ethylbutanoyl)amino]-2-hydroxyphenyl}-N-(4- |
| | methylbenzyl)-1H-pyrazole-1-carboxamide |
| 1081 | N-(2-chlorobenzyl)-5-cyclopropyl-3-{4-[(2-ethylbutanoyl)amino]-2- |
| | hydroxyphenyl}-1H-pyrazole-1-carboxamide |
| 1082 | 5-cyclopropyl-3-{4-[(2-ethylbutanoyl)amino]-2-hydroxyphenyl}-N-(4- |
| | fluorobenzyl)-1H-pyrazole-1-carboxamide |
| 1083 | 5-cyclopropyl-N-(3,4-dichlorobenzyl)-3-{4-[(2-ethylbutanoyl)amino]-2- |
| | hydroxyphenyl}-1H-pyrazole-1-carboxamide |
| 1084 | 5-cyclopropyl-3-{4-[(2-ethylbutanoyl)amino]-2-hydroxyphenyl}-N-(4- |
| | methoxybenzyl)-1H-pyrazole-1-carboxamide |
| 1085 | N-(sec-butyl)-5-cyclopropyl-3-{4-[(2-ethylbutanoyl)amino]-2-hydroxyphenyl}- |
| | 1H-pyrazole-1-carboxamide |
| 1086 | 5-cyclopropyl-3-{4-[(2-ethylbutanoyl)amino]-2-hydroxyphenyl}-N-(2- |
| | fluorobenzyl)-1H-pyrazole-1-carboxamide |
| 1087 | 5-cyclopropyl-3-{4-[(2-ethylbutanoyl)amino]-2-hydroxyphenyl}-N-(3- |
| | fluorobenzyl)-1H-pyrazole-1-carboxamide |
| 1088 | N,5-dicyclopropyl-3-{4-[(2-ethylbutanoyl)amino]-2-hydroxyphenyl}-1H- |
| | pyrazole-1-carboxamide |
| 1089 | 5-cyclobutyl-3-{4-[(2-ethylbutanoyl)amino]-2-hydroxyphenyl}-N-isopropyl-1H- |
| | pyrazole-1-carboxamide |
| 1090 | 5-cyclobutyl-N-ethyl-3-{4-[(2-ethylbutanoyl)amino]-2-hydroxyphenyl}-1H- |
| | pyrazole-1-carboxamide |
| 1091 | N-allyl-5-cyclobutyl-3-{4-[(2-ethylbutanoyl)amino]-2-hydroxyphenyl}-1H- |
| | pyrazole-1-carboxamide |
| 1092 | 5-cyclobutyl-3-{4-[(2-ethylbutanoyl)amino]-2-hydroxyphenyl}-N-propyl-1H- |
| | pyrazole-1-carboxamide |
| 1093 | N-benzyl-5-cyclobutyl-3-{4-[(2-ethylbutanoyl)amino]-2-hydroxyphenyl}-1H- |
| | pyrazole-1-carboxamide |
| 1094 | 5-cyclobutyl-3-{4-[(2-ethylbutanoyl)amino]-2-hydroxyphenyl}-N-(2- |
| | phenylethyl)-1H-pyrazole-1-carboxamide |
| 1095 | 5-cyclobutyl-3-{4-[(2-ethylbutanoyl)amino]-2-hydroxyphenyl}-N-(2- |
| | methylbenzyl)-1H-pyrazole-1-carboxamide |
| 1096 | 5-cyclobutyl-3-{4-[(2-ethylbutanoyl)amino]-2-hydroxyphenyl}-N-(3- |
| | methylbenzyl)-1H-pyrazole-1-carboxamide |
| 1097 | 5-cyclobutyl-3-{4-[(2-ethylbutanoyl)amino]-2-hydroxyphenyl}-N-(4- |
| | methylbenzyl)-1H-pyrazole-1-carboxamide |
| 1098 | N-(2-chlorobenzyl)-5-cyclobutyl-3-{4-[(2-ethylbutanoyl)amino]-2- |
| | hydroxyphenyl}-1H-pyrazole-1-carboxamide |
| 1099 | 5-cyclobutyl-3-{4-[(2-ethylbutanoyl)amino]-2-hydroxyphenyl}-N-(4- |
| | fluorobenzyl)-1H-pyrazole-1-carboxamide |
| 1100 | 5-cyclobutyl-N-(3,4-dichlorobenzyl)-3-{4-[(2-ethylbutanoyl)amino]-2- |
| | hydroxyphenyl}-1H-pyrazole-1-carboxamide |
| 1101 | 5-cyclobutyl-3-{4-[(2-ethylbutanoyl)amino]-2-hydroxyphenyl}-N-(4- |
| | methoxybenzyl)-1H-pyrazole-1-carboxamide |
| 1102 | N-(sec-butyl)-5-cyclobutyl-3-{4-[(2-ethylbutanoyl)amino]-2-hydroxyphenyl}-1H- |
| | pyrazole-1-carboxamide |
| 1103 | 5-cyclobutyl-3-{4-[(2-ethylbutanoyl)amino]-2-hydroxyphenyl}-N-(2- |
| | fluorobenzyl)-1H-pyrazole-1-carboxamide |
| 1104 | 5-cyclobutyl-3-{4-[(2-ethylbutanoyl)amino]-2-hydroxyphenyl}-N-(3- |
| | fluorobenzyl)-1H-pyrazole-1-carboxamide |
| 1105 | 5-cyclobutyl-N-cyclopropyl-3-{4-[(2-ethylbutanoyl)amino]-2-hydroxyphenyl}- |
| | 1H-pyrazole-1-carboxamide |
| 1106 | 5-cyclopentyl-3-{4-[(2-ethylbutanoyl)amino]-2-hydroxyphenyl}-N-isopropyl-1H- |
| | pyrazole-1-carboxamide |
| 1107 | 5-cyclopentyl-N-ethyl-3-{4-[(2-ethylbutanoyl)amino]-2-hydroxyphenyl}-1H- |
| | pyrazole-1-carboxamide |
| 1108 | N-allyl-5-cyclopentyl-3-{4-[(2-ethylbutanoyl)amino]-2-hydroxyphenyl}-1H- |
| | pyrazole-1-carboxamide |
| 1109 | 5-cyclopentyl-3-{4-[(2-ethylbutanoyl)amino]-2-hydroxyphenyl}-N-propyl-1H- |
| | pyrazole-1-carboxamide |
| 1110 | N-benzyl-5-cyclopentyl-3-{4-[(2-ethylbutanoyl)amino]-2-hydroxyphenyl}-1H- |
| | pyrazole-1-carboxamide |
| 1111 | 5-cyclopentyl-3-{4-[(2-ethylbutanoyl)amino]-2-hydroxyphenyl}-N-(2- |
| | phenylethyl)-1H-pyrazole-1-carboxamide |
| 1112 | 5-cyclopentyl-3-{4-[(2-ethylbutanoyl)amino]-2-hydroxyphenyl}-N-(2- |
| | methylbenzyl)-1H-pyrazole-1-carboxamide |
| 1113 | 5-cyclopentyl-3-{4-[(2-ethylbutanoyl)amino]-2-hydroxyphenyl}-N-(3- |
| | methylbenzyl)-1H-pyrazole-1-carboxamide |
| 1114 | 5-cyclopentyl-3-{4-[(2-ethylbutanoyl)amino]-2-hydroxyphenyl}-N-(4- |
| | methylbenzyl)-1H-pyrazole-1-carboxamide |
| 1115 | N-(2-chlorobenzyl)-5-cyclopentyl-3-{4-[(2-ethylbutanoyl)amino]-2- |
| | hydroxyphenyl}-1H-pyrazole-1-carboxamide |
| 1116 | 5-cyclopentyl-3-{4-[(2-ethylbutanoyl)amino]-2-hydroxyphenyl}-N-(4- |
| | fluorobenzyl)-1H-pyrazole-1-carboxamide |
| 1117 | 5-cyclopentyl-N-(3,4-dichlorobenzyl)-3-{4-[(2-ethylbutanoyl)amino]-2- |
| | hydroxyphenyl}-1H-pyrazole-1-carboxamide |
| 1118 | 5-cyclopentyl-3-{4-[(2-ethylbutanoyl)amino]-2-hydroxyphenyl}-N-(4- |
| | methoxybenzyl)-1H-pyrazole-1-carboxamide |
| 1119 | N-(sec-butyl)-5-cyclopentyl-3-{4-[(2-ethylbutanoyl)amino]-2-hydroxyphenyl}- |
| | 1H-pyrazole-1-carboxamide |
| 1120 | 5-cyclopentyl-3-{4-[(2-ethylbutanoyl)amino]-2-hydroxyphenyl}-N-(2- |
| | fluorobenzyl)-1H-pyrazole-1-carboxamide |
| 1121 | 5-cyclopentyl-3-{4-[(2-ethylbutanoyl)amino]-2-hydroxyphenyl}-N-(3- |
| | fluorobenzyl)-1H-pyrazole-1-carboxamide |
| 1122 | 5-cyclopentyl-N-cyclopropyl-3-{4-[(2-ethylbutanoyl)amino]-2-hydroxyphenyl}- |
| | 1H-pyrazole-1-carboxamide |
| 1123 | 3-{4-[(1-benzothien-2-ylcarbonyl)amino]-2-hydroxyphenyl}-5-cyclopropyl-N- |
| | isopropyl-1H-pyrazole-1-carboxamide |
| 1124 | 3-{4-[(1-benzothien-2-ylcarbonyl)amino]-2-hydroxyphenyl}-5-cyclopropyl-N- |
| | ethyl-1H-pyrazole-1-carboxamide |
| 1125 | N-allyl-3-{4-[(1-benzothien-2-ylcarbonyl)amino]-2-hydroxyphenyl}-5- |
| | cyclopropyl-1H-pyrazole-1-carboxamide |
| 1126 | 3-{4-[(1-benzothien-2-ylcarbonyl)amino]-2-hydroxyphenyl}-5-cyclopropyl-N- |
| | propyl-1H-pyrazole-1-carboxamide |
| 1127 | 3-{4-[(1-benzothien-2-ylcarbonyl)amino]-2-hydroxyphenyl}-N-benzyl-5- |
| | cyclopropyl-1H-pyrazole-1-carboxamide |
| 1128 | 3-{4-[(1-benzothien-2-ylcarbonyl)amino]-2-hydroxyphenyl}-5-cyclopropyl-N-(2- |
| | phenylethyl)-1H-pyrazole-1-carboxamide |
| 1129 | 3-{4-[(1-benzothien-2-ylcarbonyl)amino]-2-hydroxyphenyl}-5-cyclopropyl-N-(2- |
| | methylbenzyl)-1H-pyrazole-1-carboxamide |
| 1130 | 3-{4-[(1-benzothien-2-ylcarbonyl)amino]-2-hydroxyphenyl}-5-cyclopropyl-N-(3- |
| | methylbenzyl)-1H-pyrazole-1-carboxamide |
| 1131 | 3-{4-[(1-benzothien-2-ylcarbonyl)amino]-2-hydroxyphenyl}-5-cyclopropyl-N-(4- |
| | methylbenzyl)-1H-pyrazole-1-carboxamide |
| 1132 | 3-{4-[(1-benzothien-2-ylcarbonyl)amino]-2-hydroxyphenyl}-N-(2-chlorobenzyl)- |
| | 5-cyclopropyl-1H-pyrazole-1-carboxamide |
| 1133 | 3-{4-[(1-benzothien-2-ylcarbonyl)amino]-2-hydroxyphenyl}-5-cyclopropyl-N-(4- |
| | fluorobenzyl)-1H-pyrazole-1-carboxamide |
| 1134 | 3-{4-[(1-benzothien-2-ylcarbonyl)amino]-2-hydroxyphenyl}-5-cyclopropyl-N- |
| | (3,4-dichlorobenzyl)-1H-pyrazole-1-carboxamide |
| 1135 | 3-{4-[(1-benzothien-2-ylcarbonyl)amino]-2-hydroxyphenyl}-5-cyclopropyl-N-(4- |
| | methoxybenzyl)-1H-pyrazole-1-carboxamide |
| 1136 | 3-{4-[(1-benzothien-2-ylcarbonyl)amino]-2-hydroxyphenyl}-N-(sec-butyl)-5- |
| | cyclopropyl-1H-pyrazole-1-carboxamide |
| 1137 | 3-{4-[(1-benzothien-2-ylcarbonyl)amino]-2-hydroxyphenyl}-5-cyclopropyl-N-(2- |
| | fluorobenzyl)-1H-pyrazole-1-carboxamide |
| 1138 | 3-{4-[(1-benzothien-2-ylcarbonyl)amino]-2-hydroxyphenyl}-5-cyclopropyl-N-(3- |
| | fluorobenzyl)-1H-pyrazole-1-carboxamide |
| 1139 | 3-{4-[(1-benzothien-2-ylcarbonyl)amino]-2-hydroxyphenyl}-N,5-dicyclopropyl- |
| | 1H-pyrazole-1-carboxamide |
| 1140 | 3-{4-[(1-benzothien-2-ylcarbonyl)amino]-2-hydroxyphenyl}-5-cyclobutyl-N- |
| | isopropyl-1H-pyrazole-1-carboxamide |
| 1141 | 3-{4-[(1-benzothien-2-ylcarbonyl)amino]-2-hydroxyphenyl}-5-cyclobutyl-N- |
| | ethyl-1H-pyrazole-1-carboxamide |
| 1142 | N-allyl-3-{4-[(1-benzothien-2-ylcarbonyl)amino]-2-hydroxyphenyl}-5- |
| | cyclobutyl-1H-pyrazole-1-carboxamide |
| 1143 | 3-{4-[(1-benzothien-2-ylcarbonyl)amino]-2-hydroxyphenyl}-5-cyclobutyl-N- |
| | propyl-1H-pyrazole-1-carboxamide |
| 1144 | 3-{4-[(1-benzothien-2-ylcarbonyl)amino]-2-hydroxyphenyl}-N-benzyl-5- |
| | cyclobutyl-1H-pyrazole-1-carboxamide |
| 1145 | 3-{4-[(1-benzothien-2-ylcarbonyl)amino]-2-hydroxyphenyl}-5-cyclobutyl-N-(2- |
| | phenylethyl)-1H-pyrazole-1-carboxamide |
| 1146 | 3-{4-[(1-benzothien-2-ylcarbonyl)amino]-2-hydroxyphenyl}-5-cyclobutyl-N-(2- |
| | methylbenzyl)-1H-pyrazole-1-carboxamide |
| 1147 | 3-{4-[(1-benzothien-2-ylcarbonyl)amino]-2-hydroxyphenyl}-5-cyclobutyl-N-(3- |
| | methylbenzyl)-1H-pyrazole-1-carboxamide |
| 1148 | 3-{4-[(1-benzothien-2-ylcarbonyl)amino]-2-hydroxyphenyl}-5-cyclobutyl-N-(4- |
| | methylbenzyl)-1H-pyrazole-1-carboxamide |
| 1149 | 3-{4-[(1-benzothien-2-ylcarbonyl)amino]-2-hydroxyphenyl}-N-(2-chlorobenzyl)- |
| | 5-cyclobutyl-1H-pyrazole-1-carboxamide |
| 1150 | 3-{4-[(1-benzothien-2-ylcarbonyl)amino]-2-hydroxyphenyl}-5-cyclobutyl-N-(4- |
| | fluorobenzyl)-1H-pyrazole-1-carboxamide |
| 1151 | 3-{4-[(1-benzothien-2-ylcarbonyl)amino]-2-hydroxyphenyl}-5-cyclobutyl-N-(3,4- |
| | dichlorobenzyl)-1H-pyrazole-1-carboxamide |
| 1152 | 3-{4-[(1-benzothien-2-ylcarbonyl)amino]-2-hydroxyphenyl}-5-cyclobutyl-N-(4- |
| | methoxybenzyl)-1H-pyrazole-1-carboxamide |
| 1153 | 3-{4-[(1-benzothien-2-ylcarbonyl)amino]-2-hydroxyphenyl}-5-cyclobutyl-N-(2- |
| | fluorobenzyl)-1H-pyrazole-1-carboxamide |
| 1154 | 3-{4-[(1-benzothien-2-ylcarbonyl)amino]-2-hydroxyphenyl}-5-cyclobutyl-N-(2- |
| | fluorobenzyl)-1H-pyrazole-1-carboxamide |
| 1155 | 3-{4-[(1-benzothien-2-ylcarbonyl)amino]-2-hydroxyphenyl}-5-cyclobutyl-N-(3- |
| | fluorobenzyl)-1H-pyrazole-1-carboxamide |
| 1156 | 3-{4-[(1-benzothien-2-ylcarbonyl)amino]-2-hydroxyphenyl}-5-cyclobutyl-N- |
| | cyclopropyl-1H-pyrazole-1-carboxamide |
| 1157 | 3-{4-[(1-benzothien-2-ylcarbonyl)amino]-2-hydroxyphenyl}-5-cyclopentyl-N- |
| | isopropyl-1H-pyrazole-1-carboxamide |
| 1158 | 3-{4-[(1-benzothien-2-ylcarbonyl)amino]-2-hydroxyphenyl}-5-cyclopentyl-N- |
| | ethyl-1H-pyrazole-1-carboxamide |
| 1159 | N-allyl-3-{4-[(1-benzothien-2-ylcarbonyl)amino]-2-hydroxyphenyl}-5- |
| | cyclopentyl-1H-pyrazole-1-carboxamide |
| 1160 | 3-{4-[(1-benzothien-2-ylcarbonyl)amino]-2-hydroxyphenyl}-5-cyclopentyl-N- |
| | propyl-1H-pyrazole-1-carboxamide |
| 1161 | 3-{4-[(1-benzothien-2-ylcarbonyl)amino]-2-hydroxyphenyl}-N-benzyl-5- |
| | cyclopentyl-1H-pyrazole-1-carboxamide |
| 1162 | 3-{4-[(1-benzothien-2-ylcarbonyl)amino]-2-hydroxyphenyl}-5-cyclopentyl-N-(2- |
| | phenylethyl)-1H-pyrazole-1-carboxamide |
| 1163 | 3-{4-[(1-benzothien-2-ylcarbonyl)amino]-2-hydroxyphenyl}-5-cyclopentyl-N-(2- |
| | methylbenzyl)-1H-pyrazole-1-carboxamide |
| 1164 | 3-{4-[(1-benzothien-2-ylcarbonyl)amino]-2-hydroxyphenyl}-5-cyclopentyl-N-(3- |
| | methylbenzyl)-1H-pyrazole-1-carboxamide |
| 1165 | 3-{4-[(1-benzothien-2-ylcarbonyl)amino]-2-hydroxyphenyl}-5-cyclopentyl-N-(4- |
| | methylbenzyl)-1H-pyrazole-1-carboxamide |
| 1166 | 3-{4-[(1-benzothien-2-ylcarbonyl)amino]-2-hydroxyphenyl}-N-(2-chlorobenzyl)- |
| | 5-cyclopentyl-1H-pyrazole-1-carboxamide |
| 1167 | 3-{4-[(1-benzothien-2-ylcarbonyl)amino]-2-hydroxyphenyl}-5-cyclopentyl-N-(4- |
| | fluorobenzyl)-1H-pyrazole-1-carboxamide |
| 1168 | 3-{4-[(1-benzothien-2-ylcarbonyl)amino]-2-hydroxyphenyl}-5-cyclopentyl-N- |
| | (3,4-dichlorobenzyl)-1H-pyrazole-1-carboxamide |
| 1169 | 3-{4-[(1-benzothien-2-ylcarbonyl)amino]-2-hydroxyphenyl}-5-cyclopentyl-N-(4- |
| | methoxybenzyl)-1H-pyrazole-1-carboxamide |
| 1170 | 3-{4-[(1-benzothien-2-ylcarbonyl)amino]-2-hydroxyphenyl}-N-(sec-butyl)-5- |
| | cyclopentyl-1H-pyrazole-1-carboxamide |
| 1171 | 3-{4-[(1-benzothien-2-ylcarbonyl)amino]-2-hydroxyphenyl}-5-cyclopentyl-N-(2- |
| | fluorobenzyl)-1H-pyrazole-1-carboxamide |
| 1172 | 3-{4-[(1-benzothien-2-ylcarbonyl)amino]-2-hydroxyphenyl}-5-cyclopentyl-N-(3- |
| | fluorobenzyl)-1H-pyrazole-1-carboxamide |
| 1173 | 3-{4-[(1-benzothien-2-ylcarbonyl)amino]-2-hydroxyphenyl}-5-cyclopentyl-N- |
| | cyclopropyl-1H-pyrazole-1-carboxamide |
| 1174 | 2-chloro-N-(4-{5-cyclopropyl-1-[(isopropylamino)carbonyl]-1H-pyrazol-3-yl}-3- |
| | hydroxyphenyl)nicotinamide |
| 1175 | 2-chloro-N-(4-{5-cyclopropyl-1-[(ethylamino)carbonyl]-1H-pyrazol-3-yl}-3- |
| | hydroxyphenyl)nicotinamide |
| 1176 | N-(4-{1-[(allylamino)carbonyl]-5-cyclopropyl-1H-pyrazol-3-yl}-3- |
| | hydroxyphenyl)-2-chloronicotinamide |
| 1177 | 2-chloro-N-(4-{5-cyclopropyl-1-[(propylamino)carbonyl]-1H-pyrazol-3-yl}-3- |
| | hydroxyphenyl)nicotinamide |
| 1178 | N-(4-{1-[(benzylamino)carbonyl]-5-cyclopropyl-1H-pyrazol-3-yl}-3- |
| | hydroxyphenyl)-2-chloronicotinamide |
| 1179 | 2-chloro-N-[4-(5-cyclopropyl-1-{[(2-phenylethyl)amino]carbonyl}-1H-pyrazol-3- |
| | yl)-3-hydroxyphenyl]nicotinamide |
| 1180 | 2-chloro-N-[4-(5-cyclopropyl-1-{[(2-methylbenzyl)amino]carbonyl}-1H-pyrazol- |
| | 3-yl)-3-hydroxyphenyl]nicotinamide |
| 1181 | 2-chloro-N-[4-(5-cyclopropyl-1-{[(3-methylbenzyl)amino]carbonyl}-1H-pyrazol- |
| | 3-yl)-3-hydroxyphenyl]nicotinamide |
| 1182 | 2-chloro-N-[4-(5-cyclopropyl-1-{[(4-methylbenzyl)amino]carbonyl}-1H-pyrazol- |
| | 3-yl)-3-hydroxyphenyl]nicotinamide |
| 1183 | 2-chloro-N-[4-(1-{[(2-chlorobenzyl)amino]carbonyl}-5-cyclopropyl-1H-pyrazol- |
| | 3-yl)-3-hydroxyphenyl]nicotinamide |
| 1184 | 2-chloro-N-[4-(5-cyclopropyl-1-{[(4-fluorobenzyl)amino]carbonyl}-1H-pyrazol- |
| | 3-yl)-3-hydroxyphenyl]nicotinamide |
| 1185 | 2-chloro-N-[4-(5-cyclopropyl-1-{[(3,4-dichlorobenzyl)amino]carbonyl}-1H- |
| | pyrazol-3-yl)-3-hydroxyphenyl]nicotinamide |
| 1186 | 2-chloro-N-[4-(5-cyclopropyl-1-{[(4-methoxybenzyl)amino]carbonyl}-1H- |
| | pyrazol-3-yl)-3-hydroxyphenyl]nicotinamide |
| 1187 | N-(4-{1-[(sec-butylamino)carbonyl]-5-cyclopropyl-1H-pyrazol-3-yl}-3- |
| | hydroxyphenyl)-2-chloronicotinamide |
| 1188 | 2-chloro-N-[4-(5-cyclopropyl-1-{[(2-fluorobenzyl)amino]carbonyl}-1H-pyrazol- |
| | 3-yl)-3-hydroxyphenyl]nicotinamide |
| 1189 | 2-chloro-N-[4-(5-cyclopropyl-1-{[(3-fluorobenzyl)amino]carbonyl}-1H-pyrazol- |
| | 3-yl)-3-hydroxyphenyl]nicotinamide |
| 1190 | 2-chloro-N-(4-{5-cyclopropyl-1-[(cyclopropylamino)carbonyl]-1H-pyrazol-3-yl}- |
| | 3-hydroxyphenyl)nicotinamide |
| 1191 | 2-chloro-N-(4-{5-cyclobutyl-1-[(isopropylamino)carbonyl]-1H-pyrazol-3-yl}-3- |
| | hydroxyphenyl)nicotinamide |
| 1192 | 2-chloro-N-(4-{5-cyclobutyl-1-[(ethylamino)carbonyl]-1H-pyrazol-3-yl}-3- |
| | hydroxyphenyl)nicotinamide |
| 1193 | N-(4-{1-[(allylamino)carbonyl]-5-cyclobutyl-1H-pyrazol-3-yl}-3- |
| | hydroxyphenyl)-2-chloronicotinamide |
| 1194 | 2-chloro-N-(4-{5-cyclobutyl-1-[(propylamino)carbonyl]-1H-pyrazol-3-yl}-3- |
| | hydroxyphenyl)nicotinamide |
| 1195 | N-(4-{1-[(benzylamino)carbonyl]-5-cyclobutyl-1H-pyrazol-3-yl}-3- |
| | hydroxyphenyl)-2-chloronicotinamide |
| 1196 | 2-chloro-N-[4-(5-cyclobutyl-1-{[(2-phenylethyl)amino]carbonyl}-1H-pyrazol-3- |
| | yl)-3-hydroxyphenyl]nicotinamide |
| 1197 | 2-chloro-N-[4-(5-cyclobutyl-1-{[(2-methylbenzyl)amino]carbonyl}-1H-pyrazol- |
| | 3-yl)-3-hydroxyphenyl]nicotinamide |
| 1198 | 2-chloro-N-[4-(5-cyclobutyl-1-{[(3-methylbenzyl)amino]carbonyl}-1H-pyrazol- |
| | 3-yl)-3-hydroxyphenyl]nicotinamide |
| 1199 | 2-chloro-N-[4-(5-cyclobutyl-1-{[(4-methylbenzyl)amino]carbonyl}-1H-pyrazol- |
| | 3-yl)-3-hydroxyphenyl]nicotinamide |
| 1200 | 2-chloro-N-[4-(1-{[(2-chlorobenzyl)amino]carbonyl}-5-cyclobutyl-1H-pyrazol-3- |
| | yl)-3-hydroxyphenyl]nicotinamide |
| 1201 | 2-chloro-N-[4-(5-cyclobutyl-1-{[(4-fluorobenzyl)amino]carbonyl}-1H-pyrazol-3- |
| | yl)-3-hydroxyphenyl]nicotinamide |
| 1202 | 2-chloro-N-[4-(5-cyclobutyl-1-{[(3,4-dichlorobenzyl)amino]carbonyl}-1H- |
| | pyrazol-3-yl)-3-hydroxyphenyl]nicotinamide |
| 1203 | 2-chloro-N-[4-(5-cyclobutyl-1-{[(4-methoxybenzyl)amino]carbonyl}-1H-pyrazol- |
| | 3-yl)-3-hydroxyphenyl]nicotinamide |
| 1204 | N-(4-{1-[(sec-butylamino)carbonyl]-5-cyclobutyl-1H-pyrazol-3-yl}-3- |
| | hydroxyphenyl)-2-chloronicotinamide |
| 1205 | 2-chloro-N-[4-(5-cyclobutyl-1-{[(2-fluorobenzyl)amino]carbonyl}-1H-pyrazol-3- |
| | yl)-3-hydroxyphenyl]nicotinamide |
| 1206 | 2-chloro-N-[4-(5-cyclobutyl-1-{[(3-fluorobenzyl)amino]carbonyl}-1H-pyrazol-3- |
| | yl)-3-hydroxyphenyl]nicotinamide |
| 1207 | 2-chloro-N-(4-{5-cyclobutyl-1-[(cyclopropylamino)carbonyl]-1H-pyrazol-3-yl}- |
| | 3-hydroxyphenyl)nicotinamide |
| 1208 | 2-chloro-N-(4-{5-cyclopentyl-1-[(isopropylamino)carbonyl]-1H-pyrazol-3-yl}-3- |
| | hydroxyphenyl)nicotinamide |
| 1209 | 2-chloro-N-(4-{5-cyclopentyl-1-[(ethylamino)carbonyl]-1H-pyrazol-3-yl}-3- |
| | hydroxyphenyl)nicotinamide |
| 1210 | N-(4-{1-[(allylamino)carbonyl]-5-cyclopentyl-1H-pyrazol-3-yl}-3- |
| | hydroxyphenyl)-2-chloronicotinamide |
| 1211 | 2-chloro-N-(4-{5-cyclopentyl-1-[(propylamino)carbonyl]-1H-pyrazol-3-yl}-3- |
| | hydroxyphenyl)nicotinamide |
| 1212 | N-(4-{1-[(benzylamino)carbonyl]-5-cyclopentyl-1H-pyrazol-3-yl}-3- |
| | hydroxyphenyl)-2-chloronicotinamide |
| 1213 | 2-chloro-N-[4-(5-cyclopentyl-1-{[(2-phenylethyl)amino]carbonyl}-1H-pyrazol-3- |
| | yl)-3-hydroxyphenyl]nicotinamide |
| 1214 | 2-chloro-N-[4-(5-cyclopentyl-1-{[(2-methylbenzyl)amino]carbonyl}-1H-pyrazol- |
| | 3-yl)-3-hydroxyphenyl]nicotinamide |
| 1215 | 2-chloro-N-[4-(5-cyclopentyl-1-{[(3-methylbenzyl)amino]carbonyl}-1H-pyrazol- |
| | 3-yl)-3-hydroxyphenyl]nicotinamide |
| 1216 | 2-chloro-N-[4-(5-cyclopentyl-1-{[(4-methylbenzyl)amino]carbonyl}-1H-pyrazol- |
| | 3-yl)-3-hydroxyphenyl]nicotinamide |
| 1217 | 2-chloro-N-[4-(1-{[(2-chlorobenzyl)amino]carbonyl}-5-cyclopentyl-1H-pyrazol- |
| | 3-yl)-3-hydroxyphenyl]nicotinamide |
| 1218 | 2-chloro-N-[4-(5-cyclopentyl-1-{[(4-fluorobenzyl)amino]carbonyl}-1H-pyrazol- |
| | 3-yl)-3-hydroxyphenyl]nicotinamide |
| 1219 | 2-chloro-N-[4-(5-cyclopentyl-1-{[(3,4-dichlorobenzyl)amino]carbonyl}-1H- |
| | pyrazol-3-yl)-3-hydroxyphenyl]nicotinamide |
| 1220 | 2-chloro-N-[4-(5-cyclopentyl-1-{[(4-methoxybenzyl)amino]carbonyl}-1H- |
| | pyrazol-3-yl)-3-hydroxyphenyl]nicotinamide |
| 1221 | N-(4-{1-[(sec-butylamino)carbonyl]-5-cyclopentyl-1H-pyrazol-3-yl}-3- |
| | hydroxyphenyl)-2-chloronicotinamide |
| 1222 | 2-chloro-N-[4-(5-cyclopentyl-1-{[(2-fluorobenzyl)amino]carbonyl}-1H-pyrazol- |
| | 3-yl)-3-hydroxyphenyl]nicotinamide |
| 1223 | 2-chloro-N-[4-(5-cyclopentyl-1-{[(3-fluorobenzyl)amino]carbonyl}-1H-pyrazol- |
| | 3-yl)-3-hydroxyphenyl]nicotinamide |
| 1224 | 2-chloro-N-(4-{5-cyclopentyl-1-[(cyclopropylamino)carbonyl]-1H-pyrazol-3-yl}- |
| | 3-hydroxyphenyl)nicotinamide |
| 1225 | 5-cyclopropyl-3-(2-hydroxy-4-{[4-(trifluoromethoxy)benzoyl]amino}phenyl)-N- |
| | isopropyl-1H-pyrazole-1-carboxamide |
| 1226 | 5-cyclopropyl-N-ethyl-3-(2-hydroxy-4-{[4- |
| | (trifluoromethoxy)benzoyl]amino}phenyl)-1H-pyrazole-1-carboxamide |
| 1227 | N-allyl-5-cyclopropyl-3-(2-hydroxy-4-{[4- |
| | (trifluoromethoxy)benzoyl]amino}phenyl)-1H-pyrazole-1-carboxamide |
| 1228 | 5-cyclopropyl-3-(2-hydroxy-4-{[4-(trifluoromethoxy)benzoyl]amino}phenyl)-N- |
| | propyl-1H-pyrazole-1-carboxamide |
| 1229 | N-benzyl-5-cyclopropyl-3-(2-hydroxy-4-{[4- |
| | (trifluoromethoxy)benzoyl]amino}phenyl)-1H-pyrazole-1-carboxamide |
| 1230 | 5-cyclopropyl-3-(2-hydroxy-4-{[4-(trifluoromethoxy)benzoyl]amino}phenyl)-N- |
| | (2-phenylethyl)-1H-pyrazole-1-carboxamide |
| 1231 | 5-cyclopropyl-3-(2-hydroxy-4-{[4-(trifluoromethoxy)benzoyl]amino}phenyl)-N- |
| | (2-methylbenzyl)-1H-pyrazole-1-carboxamide |
| 1232 | 5-cyclopropyl-3-(2-hydroxy-4-{[4-(trifluoromethoxy)benzoyl]amino}phenyl)-N- |
| | (3-methylbenzyl)-1H-pyrazole-1-carboxamide |
| 1233 | 5-cyclopropyl-3-(2-hydroxy-4-{[4-(trifluoromethoxy)benzoyl]amino}phenyl)-N- |
| | (4-methylbenzyl)-1H-pyrazole-1-carboxamide |
| 1234 | N-(2-chlorobenzyl)-5-cyclopropyl-3-(2-hydroxy-4-{[4- |
| | (trifluoromethoxy)benzoyl]amino}phenyl)-1H-pyrazole-1-carboxamide |
| 1235 | 5-cyclopropyl-N-(4-fluorobenzyl)-3-(2-hydroxy-4-{[4- |
| | (trifluoromethoxy)benzoyl]amino}phenyl)-1H-pyrazole-1-carboxamide |
| 1236 | 5-cyclopropyl-N-(3,4-dichlorobenzyl)-3-(2-hydroxy-4-{[4- |
| | (trifluoromethoxy)benzoyl]amino}phenyl)-1H-pyrazole-1-carboxamide |
| 1237 | 5-cyclopropyl-3-(2-hydroxy-4-{[4-(trifluoromethoxy)benzoyl]amino}phenyl)-N- |
| | (4-methoxybenzyl)-1H-pyrazole-1-carboxamide |
| 1238 | N-(sec-butyl)-5-cyclopropyl-3-(2-hydroxy-4-{[4- |
| | (trifluoromethoxy)benzoyl]amino}phenyl)-1H-pyrazole-1-carboxamide |
| 1239 | 5-cyclopropyl-N-(2-fluorobenzyl)-3-(2-hydroxy-4-{[4- |
| | (trifluoromethoxy)benzoyl]amino}phenyl)-1H-pyrazole-1-carboxamide |
| 1240 | 5-cyclopropyl-N-(3-fluorobenzyl)-3-(2-hydroxy-4-{[4- |
| | (trifluoromethoxy)benzoyl]amino}phenyl)-1H-pyrazole-1-carboxamide |
| 1241 | N,5-dicyclopropyl-3-(2-hydroxy-4-{[4- |
| | (trifluoromethoxy)benzoyl]amino}phenyl)-1H-pyrazole-1-carboxamide |
| 1242 | 5-cyclobutyl-N-ethyl-3-(2-hydroxy-4-{[4- |
| | (trifluoromethoxy)benzoyl]amino}phenyl)-1H-pyrazole-1-carboxamide |
| 1243 | 5-cyclobutyl-N-ethyl-3-(2-hydroxy-4-{[4- |
| | (trifluoromethoxy)benzoyl]amino}phenyl)-1H-pyrazole-1-carboxamide |
| 1244 | N-allyl-5-cyclobutyl-3-(2-hydroxy-4-{[4- |
| | (trifluoromethoxy)benzoyl]amino}phenyl)-1H-pyrazole-1-carboxamide |
| 1245 | 5-cyclobutyl-3-(2-hydroxy-4-{[4-(trifluoromethoxy)benzoyl]amino}phenyl)-N- |
| | propyl-1H-pyrazole-1-carboxamide |
| 1246 | N-benzyl-5-cyclobutyl-3-(2-hydroxy-4-{[4- |
| | (trifluoromethoxy)benzoyl]amino}phenyl)-1H-pyrazole-1-carboxamide |
| 1247 | 5-cyclobutyl-3-(2-hydroxy-4-{[4-(trifluoromethoxy)benzoyl]amino}phenyl)-N- |
| | (2-phenylethyl)-1H-pyrazole-1-carboxamide |
| 1248 | 5-cyclobutyl-3-(2-hydroxy-4-{[4-(trifluoromethoxy)benzoyl]amino}phenyl)-N- |
| | (2-methylbenzyl)-1H-pyrazole-1-carboxamide |
| 1249 | 5-cyclobutyl-3-(2-hydroxy-4-{[4-(trifluoromethoxy)benzoyl]amino}phenyl)-N- |
| | (3-methylbenzyl)-1H-pyrazole-1-carboxamide |
| 1250 | 5-cyclobutyl-3-(2-hydroxy-4-{[4-(trifluoromethoxy)benzoyl]amino}phenyl)-N- |
| | (4-methylbenzyl)-1H-pyrazole-1-carboxamide |
| 1251 | N-(2-chlorobenzyl)-5-cyclobutyl-3-(2-hydroxy-4-{[4- |
| | (trifluoromethoxy)benzoyl]amino}phenyl)-1H-pyrazole-1-carboxamide |
| 1252 | 5-cyclobutyl-N-(4-fluorobenzyl)-3-(2-hydroxy-4-{[4- |
| | (trifluoromethoxy)benzoyl]amino}phenyl)-1H-pyrazole-1-carboxamide |
| 1253 | 5-cyclobutyl-N-(3,4-dichlorobenzyl)-3-(2-hydroxy-4-{[4- |
| | (trifluoromethoxy)benzoyl]amino}phenyl)-1H-pyrazole-1-carboxamide |
| 1254 | 5-cyclobutyl-3-(2-hydroxy-4-{[4-(trifluoromethoxy)benzoyl]amino}phenyl)-N- |
| | (4-methoxybenzyl)-1H-pyrazole-1-carboxamide |
| 1255 | N-(sec-butyl)-5-cyclobutyl-3-(2-hydroxy-4-{[4- |
| | (trifluoromethoxy)benzoyl]amino}phenyl)-1H-pyrazole-1-carboxamide |
| 1256 | 5-cyclobutyl-N-(2-fluorobenzyl)-3-(2-hydroxy-4-{[4- |
| | (trifluoromethoxy)benzoyl]amino}phenyl)-1H-pyrazole-1-carboxamide |
| 1257 | 5-cyclobutyl-N-(3-fluorobenzyl)-3-(2-hydroxy-4-{[4- |
| | (trifluoromethoxy)benzoyl]amino}phenyl)-1H-pyrazole-1-carboxamide |
| 1258 | N-allyl-5-cyclopentyl-3-(2-hydroxy-4-{[4- |
| | (trifluoromethoxy)benzoyl]amino}phenyl)-1H-pyrazole-1-carboxamide |
| 1259 | 5-cyclopentyl-3-(2-hydroxy-4-{[4-(trifluoromethoxy)benzoyl]amino}phenyl)-N- |
| | isopropyl-1H-pyrazole-1-carboxamide |
| 1260 | 5-cyclopentyl-N-ethyl-3-(2-hydroxy-4-{[4- |
| | (trifluoromethoxy)benzoyl]amino}phenyl)-1H-pyrazole-1-carboxamide |
| 1261 | N-allyl-5-cyclopentyl-3-(2-hydroxy-4-{[4- |
| | (trifluoromethoxy)benzoyl]amino}phenyl)-1H-pyrazole-1-carboxamide |
| 1262 | 5-cyclopentyl-3-(2-hydroxy-4-{[4-(trifluoromethoxy)benzoyl]amino}phenyl)-N- |
| | propyl-1H-pyrazole-1-carboxamide |
| 1263 | N-benzyl-5-cyclopentyl-3-(2-hydroxy-4-{[4- |
| | (trifluoromethoxy)benzoyl]amino}phenyl)-1H-pyrazole-1-carboxamide |
| 1264 | 5-cyclopentyl-3-(2-hydroxy-4-{[4-(trifluoromethoxy)benzoyl]amino}phenyl)-N- |
| | (2-phenylethyl)-1H-pyrazole-1-carboxamide |
| 1265 | 5-cyclopentyl-3-(2-hydroxy-4-{[4-(trifluoromethoxy)benzoyl]amino}phenyl)-N- |
| | (2-methylbenzyl)-1H-pyrazole-1-carboxamide |
| 1266 | 5-cyclopentyl-3-(2-hydroxy-4-{[4-(trifluoromethoxy)benzoyl]amino}phenyl)-N- |
| | (3-methylbenzyl)-1H-pyrazole-1-carboxamide |
| 1267 | 5-cyclopentyl-3-(2-hydroxy-4-{[4-(trifluoromethoxy)benzoyl]amino}phenyl)-N- |
| | (4-methylbenzyl)-1H-pyrazole-1-carboxamide |
| 1268 | N-(2-chlorobenzyl)-5-cyclopentyl-3-(2-hydroxy-4-{[4- |
| | (trifluoromethoxy)benzoyl]amino}phenyl)-1H-pyrazole-1-carboxamide |
| 1269 | 5-cyclopentyl-N-(4-fluorobenzyl)-3-(2-hydroxy-4-{[4- |
| | (trifluoromethoxy)benzoyl]amino}phenyl)-1H-pyrazole-1-carboxamide |
| 1270 | 5-cyclopentyl-N-(3,4-dichlorobenzyl)-3-(2-hydroxy-4-{[4- |
| | (trifluoromethoxy)benzoyl]amino}phenyl)-1H-pyrazole-1-carboxamide |
| 1271 | 5-cyclopentyl-3-(2-hydroxy-4-{[4-(trifluoromethoxy)benzoyl]amino}phenyl)-N- |
| | (4-methoxybenzyl)-1H-pyrazole-1-carboxamide |
| 1272 | N-(sec-butyl)-5-cyclopentyl-3-(2-hydroxy-4-{[4- |
| | (trifluoromethoxy)benzoyl]amino}phenyl)-1H-pyrazole-1-carboxamide |
| 1273 | 5-cyclopentyl-N-(2-fluorobenzyl)-3-(2-hydroxy-4-{[4- |
| | (trifluoromethoxy)benzoyl]amino}phenyl)-1H-pyrazole-1-carboxamide |
| 1274 | 5-cyclopentyl-N-(3-fluorobenzyl)-3-(2-hydroxy-4-{[4- |
| | (trifluoromethoxy)benzoyl]amino}phenyl)-1H-pyrazole-1-carboxamide |
| 1275 | 5-cyclopentyl-N-cyclopropyl-3-(2-hydroxy-4-{[4- |
| | (trifluoromethoxy)benzoyl]amino}phenyl)-1H-pyrazole-1-carboxamide |
| 1276 | N-(4-{5-cyclopropyl-1-[(isopropylamino)carbonyl]-1H-pyrazol-3-yl}-3- |
| | hydroxyphenyl)isoxazole-5-carboxamide |
| 1277 | N-(4-{5-cyclopropyl-1-[(ethylamino)carbonyl]-1H-pyrazol-3-yl}-3- |
| | hydroxyphenyl)isoxazole-5-carboxamide |
| 1278 | N-(4-{1-[(allylamino)carbonyl]-5-cyclopropyl-1H-pyrazol-3-yl}-3- |
| | hydroxyphenyl)isoxazole-5-carboxamide |
| 1279 | N-(4-{5-cyclopropyl-1-[(propylamino)carbonyl]-1H-pyrazol-3-yl}-3- |
| | hydroxyphenyl)isoxazole-5-carboxamide |
| 1280 | N-(4-{1-[(benzylamino)carbonyl]-5-cyclopropyl-1H-pyrazol-3-yl}-3- |
| | hydroxyphenyl)isoxazole-5-carboxamide |
| 1281 | N-[4-(5-cyclopropyl-1-{[(2-phenylethyl)amino]carbonyl}-1H-pyrazol-3-yl)-3- |
| | hydroxyphenyl]isoxazole-5-carboxamide |
| 1282 | N-[4-(5-cyclopropyl-1-{[(2-methylbenzyl)amino]carbonyl}-1H-pyrazol-3-yl)-3- |
| | hydroxyphenyl]isoxazole-5-carboxamide |
| 1283 | N-[4-(5-cyclopropyl-1-{[(3-methylbenzyl)amino]carbonyl}-1H-pyrazol-3-yl)-3- |
| | hydroxyphenyl]isoxazole-5-carboxamide |
| 1284 | N-[4-(5-cyclopropyl-1-{[(4-methylbenzyl)amino]carbonyl}-1H-pyrazol-3-yl)-3- |
| | hydroxyphenyl]isoxazole-5-carboxamide |
| 1285 | N-[4-(1-{[(2-chlorobenzyl)amino]carbonyl}-5-cyclopropyl-1H-pyrazol-3-yl)-3- |
| | hydroxyphenyl]isoxazole-5-carboxamide |
| 1286 | N-[4-(5-cyclopropyl-1-{[(4-fluorobenzyl)amino]carbonyl}-1H-pyrazol-3-yl)-3- |
| | hydroxyphenyl]isoxazole-5-carboxamide |
| 1287 | N-[4-(5-cyclopropyl-1-{[(3,4-dichlorobenzyl)amino]carbonyl}-1H-pyrazol-3-yl)- |
| | 3-hydroxyphenyl]isoxazole-5-carboxamide |
| 1288 | N-[4-(5-cyclopropyl-1-{[(4-methoxybenzyl)amino]carbonyl}-1H-pyrazol-3-yl)-3- |
| | hydroxyphenyl]isoxazole-5-carboxamide |
| 1289 | N-(4-{1-[(sec-butylamino)carbonyl]-5-cyclopropyl-1H-pyrazol-3-yl}-3- |
| | hydroxyphenyl)isoxazole-5-carboxamide |
| 1290 | N-[4-(5-cyclopropyl-1-{[(2-fluorobenzyl)amino]carbonyl}-1H-pyrazol-3-yl)-3- |
| | hydroxyphenyl]isoxazole-5-carboxamide |
| 1291 | N-[4-(5-cyclopropyl-1-{[(3-fluorobenzyl)amino]carbonyl}-1H-pyrazol-3-yl)-3- |
| | hydroxyphenyl]isoxazole-5-carboxamide |
| 1292 | N-(4-{5-cyclopropyl-1-[(cyclopropylamino)carbonyl]-1H-pyrazol-3-yl}-3- |
| | hydroxyphenyl)isoxazole-5-carboxamide |
| 1293 | N-(4-{5-cyclobutyl-1-[(isopropylamino)carbonyl]-1H-pyrazol-3-yl}-3- |
| | hydroxyphenyl)isoxazole-5-carboxamide |
| 1294 | N-(4-{5-cyclobutyl-1-[(ethylamino)carbonyl]-1H-pyrazol-3-yl}-3- |
| | hydroxyphenyl)isoxazole-5-carboxamide |
| 1295 | N-(4-{1-[(allylamino)carbonyl]-5-cyclobutyl-1H-pyrazol-3-yl}-3- |
| | hydroxyphenyl)isoxazole-5-carboxamide |
| 1296 | N-(4-{5-cyclobutyl-1-[(propylamino)carbonyl]-1H-pyrazol-3-yl}-3- |
| | hydroxyphenyl)isoxazole-5-carboxamide |
| 1297 | N-(4-{1-[(benzylamino)carbonyl]-5-cyclobutyl-1H-pyrazol-3-yl}-3- |
| | hydroxyphenyl)isoxazole-5-carboxamide |
| 1298 | N-[4-(5-cyclobutyl-1-{[(2-phenylethyl)amino]carbonyl}-1H-pyrazol-3-yl)-3- |
| | hydroxyphenyl]isoxazole-5-carboxamide |
| 1299 | N-[4-(5-cyclobutyl-1-{[(2-methylbenzyl)amino]carbonyl}-1H-pyrazol-3-yl)-3- |
| | hydroxyphenyl]isoxazole-5-carboxamide |
| 1300 | N-[4-(5-cyclobutyl-1-{[(3-methylbenzyl)amino]carbonyl}-1H-pyrazol-3-yl)-3- |
| | hydroxyphenyl]isoxazole-5-carboxamide |
| 1301 | N-[4-(5-cyclobutyl-1-{[(4-methylbenzyl)amino]carbonyl}-1H-pyrazol-3-yl)-3- |
| | hydroxyphenyl]isoxazole-5-carboxamide |
| 1302 | N-[4-(1-{[(2-chlorobenzyl)amino]carbonyl}-5-cyclobutyl-1H-pyrazol-3-yl)-3- |
| | hydroxyphenyl]isoxazole-5-carboxamide |
| 1303 | N-[4-(5-cyclobutyl-1-{[(4-fluorobenzyl)amino]carbonyl}-1H-pyrazol-3-yl)-3- |
| | hydroxyphenyl]isoxazole-5-carboxamide |
| 1304 | N-[4-(5-cyclobutyl-1-{[(3,4-dichlorobenzyl)amino]carbonyl}-1H-pyrazol-3-yl)-3- |
| | hydroxyphenyl]isoxazole-5-carboxamide |
| 1305 | N-[4-(5-cyclobutyl-1-{[(4-methoxybenzyl)amino]carbonyl}-1H-pyrazol-3-yl)-3- |
| | hydroxyphenyl]isoxazole-5-carboxamide |
| 1306 | N-(4-{1-[(sec-butylamino)carbonyl]-5-cyclobutyl-1H-pyrazol-3-yl}-3- |
| | hydroxyphenyl)isoxazole-5-carboxamide |
| 1307 | N-[4-(5-cyclobutyl-1-{[(2-fluorobenzyl)amino]carbonyl}-1H-pyrazol-3-yl)-3- |
| | hydroxyphenyl]isoxazole-5-carboxamide |
| 1308 | N-[4-(5-cyclobutyl-1-{[(3-fluorobenzyl)amino]carbonyl}-1H-pyrazol-3-yl)-3- |
| | hydroxyphenyl]isoxazole-5-carboxamide |
| 1309 | N-(4-{5-cyclobutyl-1-[(cyclopropylamino)carbonyl]-1H-pyrazol-3-yl}-3- |
| | hydroxyphenyl)isoxazole-5-carboxamide |
| 1310 | N-(4-{5-cyclopentyl-1-[(isopropylamino)carbonyl]-1H-pyrazol-3-yl}-3- |
| | hydroxyphenyl)isoxazole-5-carboxamide |
| 1311 | N-(4-{5-cyclopentyl-1-[(ethylamino)carbonyl]-1H-pyrazol-3-yl}-3- |
| | hydroxyphenyl)isoxazole-5-carboxamide |
| 1312 | N-(4-{1-[(allylamino)carbonyl]-5-cyclopentyl-1H-pyrazol-3-yl}-3- |
| | hydroxyphenyl)isoxazole-5-carboxamide |
| 1313 | N-(4-{5-cyclopentyl-1-[(propylamino)carbonyl]-1H-pyrazol-3-yl}-3- |
| | hydroxyphenyl)isoxazole-5-carboxamide |
| 1314 | N-(4-{1-[(benzylamino)carbonyl]-5-cyclopentyl-1H-pyrazol-3-yl}-3- |
| | hydroxyphenyl)isoxazole-5-carboxamide |
| 1315 | N-[4-(5-cyclopentyl-1-{[(2-phenylethyl)amino]carbonyl}-1H-pyrazol-3-yl)-3- |
| | hydroxyphenyl]isoxazole-5-carboxamide |
| 1316 | N-[4-(5-cyclopentyl-1-{[(2-methylbenzyl)amino]carbonyl}-1H-pyrazol-3-yl)-3- |
| | hydroxyphenyl]isoxazole-5-carboxamide |
| 1317 | N-[4-(5-cyclopentyl-1-{[(3-methylbenzyl)amino]carbonyl}-1H-pyrazol-3-yl)-3- |
| | hydroxyphenyl]isoxazole-5-carboxamide |
| 1318 | N-[4-(5-cyclopentyl-1-{[(4-methylbenzyl)amino]carbonyl}-1H-pyrazol-3-yl)-3- |
| | hydroxyphenyl]isoxazole-5-carboxamide |
| 1319 | N-[4-(1-{[(2-chlorobenzyl)amino]carbonyl}-5-cyclopentyl-1H-pyrazol-3-yl)-3- |
| | hydroxyphenyl]isoxazole-5-carboxamide |
| 1320 | N-[4-(5-cyclopentyl-1-{[(4-fluorobenzyl)amino]carbonyl}-1H-pyrazol-3-yl)-3- |
| | hydroxyphenyl]isoxazole-5-carboxamide |
| 1321 | N-[4-(5-cyclopentyl-1-{[(3,4-dichlorobenzyl)amino]carbonyl}-1H-pyrazol-3-yl)- |
| | 3-hydroxyphenyl]isoxazole-5-carboxamide |
| 1322 | N-[4-(5-cyclopentyl-1-{[(4-methoxybenzyl)amino]carbonyl}-1H-pyrazol-3-yl)-3- |
| | hydroxyphenyl]isoxazole-5-carboxamide |
| 1323 | N-(4-{1-[(sec-butylamino)carbonyl]-5-cyclopentyl-1H-pyrazol-3-yl}-3- |
| | hydroxyphenyl)isoxazole-5-carboxamide |
| 1324 | N-[4-(5-cyclopentyl-1-{[(2-fluorobenzyl)amino]carbonyl}-1H-pyrazol-3-yl)-3- |
| | hydroxyphenyl]isoxazole-5-carboxamide |
| 1325 | N-[4-(5-cyclopentyl-1-{[(3-fluorobenzyl)amino]carbonyl}-1H-pyrazol-3-yl)-3- |
| | hydroxyphenyl]isoxazole-5-carboxamide |
| 1326 | N-(4-{5-cyclopentyl-1-[(cyclopropylamino)carbonyl]-1H-pyrazol-3-yl}-3- |
| | hydroxyphenyl)isoxazole-5-carboxamide |
| 1327 | 5-cyclopropyl-3-(2-hydroxy-4-{[(1-methyl-1H-pyrrol-2- |
| | yl)carbonyl]amino}phenyl)-N-isopropyl-1H-pyrazole-1-carboxamide |
| 1328 | 5-cyclopropyl-N-ethyl-3-(2-hydroxy-4-{[(1-methyl-1H-pyrrol-2- |
| | yl)carbonyl]amino}phenyl)-1H-pyrazole-1-carboxamide |
| 1329 | N-allyl-5-cyclopropyl-3-(2-hydroxy-4-{[(1-methyl-1H-pyrrol-2- |
| | yl)carbonyl]amino}phenyl)-1H-pyrazole-1-carboxamide |
| 1330 | 5-cyclopropyl-3-(2-hydroxy-4-{[(1-methyl-1H-pyrrol-2- |
| | yl)carbonyl]amino}phenyl)-N-propyl-1H-pyrazole-1-carboxamide |
| 1331 | N-benzyl-5-cyclopropyl-3-(2-hydroxy-4-{[(1-methyl-1H-pyrrol-2- |
| | yl)carbonyl]amino}phenyl)-1H-pyrazole-1-carboxamide |
| 1332 | 5-cyclopropyl-3-(2-hydroxy-4-{[(1-methyl-1H-pyrrol-2- |
| | yl)carbonyl]amino}phenyl)-N-(2-phenylethyl)-1H-pyrazole-1-carboxamide |
| 1333 | 5-cyclopropyl-3-(2-hydroxy-4-{[(1-methyl-1H-pyrrol-2- |
| | yl)carbonyl]amino}phenyl)-N-(2-methylbenzyl)-1H-pyrazole-1-carboxamide |
| 1334 | 5-cyclopropyl-3-(2-hydroxy-4-{[(1-methyl-1H-pyrrol-2- |
| | yl)carbonyl]amino}phenyl)-N-(3-methylbenzyl)-1H-pyrazole-1-carboxamide |
| 1335 | 5-cyclopropyl-3-(2-hydroxy-4-{[(1-methyl-1H-pyrrol-2- |
| | yl)carbonyl]amino}phenyl)-N-(4-methylbenzyl)-1H-pyrazole-1-carboxamide |
| 1336 | N-(2-chlorobenzyl)-5-cyclopropyl-3-(2-hydroxy-4-{[(1-methyl-1H-pyrrol-2- |
| | yl)carbonyl]amino}phenyl)-1H-pyrazole-1-carboxamide |
| 1337 | 5-cyclopropyl-N-(4-fluorobenzyl)-3-(2-hydroxy-4-{[(1-methyl-1H-pyrrol-2- |
| | yl)carbonyl]amino}phenyl)-1H-pyrazole-1-carboxamide |
| 1338 | 5-cyclopropyl-N-(3,4-dichlorobenzyl)-3-(2-hydroxy-4-{[(1-methyl-1H-pyrrol-2- |
| | yl)carbonyl]amino}phenyl)-1H-pyrazole-1-carboxamide |
| 1339 | 5-cyclopropyl-3-(2-hydroxy-4-{[(1-methyl-1H-pyrrol-2- |
| | yl)carbonyl]amino}phenyl)-N-(4-methoxybenzyl)-1H-pyrazole-1-carboxamide |
| 1340 | N-(sec-butyl)-5-cyclopropyl-3-(2-hydroxy-4-{[(1-methyl-1H-pyrrol-2- |
| | yl)carbonyl]amino}phenyl)-1H-pyrazole-1-carboxamide |
| 1341 | 5-cyclopropyl-N-(2-fluorobenzyl)-3-(2-hydroxy-4-{[(1-methyl-1H-pyrrol-2- |
| | yl)carbonyl]amino}phenyl)-1H-pyrazole-1-carboxamide |
| 1342 | 5-cyclopropyl-N-(3-fluorobenzyl)-3-(2-hydroxy-4-{[(1-methyl-1H-pyrrol-2- |
| | yl)carbonyl]amino}phenyl)-1H-pyrazole-1-carboxamide |
| 1343 | N,5-dicyclopropyl-3-(2-hydroxy-4-{[(1-methyl-1H-pyrrol-2- |
| | yl)carbonyl]amino}phenyl)-1H-pyrazole-1-carboxamide |
| 1344 | 5-cyclobutyl-3-(2-hydroxy-4-{[(1-methyl-1H-pyrrol-2- |
| | yl)carbonyl]amino}phenyl)-N-isopropyl-1H-pyrazole-1-carboxamide |
| 1345 | 5-cyclobutyl-N-ethyl-3-(2-hydroxy-4-{[(1-methyl-1H-pyrrol-2- |
| | yl)carbonyl]amino}phenyl)-1H-pyrazole-1-carboxamide |
| 1346 | N-allyl-5-cyclobutyl-3-(2-hydroxy-4-{[(1-methyl-1H-pyrrol-2- |
| | yl)carbonyl]amino}phenyl)-1H-pyrazole-1-carboxamide |
| 1347 | 5-cyclobutyl-3-(2-hydroxy-4-{[(1-methyl-1H-pyrrol-2- |
| | yl)carbonyl]amino}phenyl)-N-propyl-1H-pyrazole-1-carboxamide |
| 1348 | N-benzyl-5-cyclobutyl-3-(2-hydroxy-4-{[(1-methyl-1H-pyrrol-2- |
| | yl)carbonyl]amino}phenyl)-1H-pyrazole-1-carboxamide |
| 1349 | 5-cyclobutyl-3-(2-hydroxy-4-{[(1-methyl-1H-pyrrol-2- |
| | yl)carbonyl]amino}phenyl)-N-(2-phenylethyl)-1H-pyrazole-1-carboxamide |
| 1350 | 5-cyclobutyl-3-(2-hydroxy-4-{[(1-methyl-1H-pyrrol-2- |
| | yl)carbonyl]amino}phenyl)-N-(2-methylbenzyl)-1H-pyrazole-1-carboxamide |
| 1351 | 5-cyclobutyl-3-(2-hydroxy-4-{[(1-methyl-1H-pyrrol-2- |
| | yl)carbonyl]amino}phenyl)-N-(3-methylbenzyl)-1H-pyrazole-1-carboxamide |
| 1352 | 5-cyclobutyl-3-(2-hydroxy-4-{[(1-methyl-1H-pyrrol-2- |
| | yl)carbonyl]amino}phenyl)-N-(4-methylbenzyl)-1H-pyrazole-1-carboxamide |
| 1353 | N-(2-chlorobenzyl)-5-cyclobutyl-3-(2-hydroxy-4-{[(1-methyl-1H-pyrrol-2- |
| | yl)carbonyl]amino}phenyl)-1H-pyrazole-1-carboxamide |
| 1354 | 5-cyclobutyl-N-(4-fluorobenzyl)-3-(2-hydroxy-4-{[(1-methyl-1H-pyrrol-2- |
| | yl)carbonyl]amino}phenyl)-1H-pyrazole-1-carboxamide |
| 1355 | 5-cyclobutyl-N-(3,4-dichlorobenzyl)-3-(2-hydroxy-4-{[(1-methyl-1H-pyrrol-2- |
| | yl)carbonyl]amino}phenyl)-1H-pyrazole-1-carboxamide |
| 1356 | 5-cyclobutyl-3-(2-hydroxy-4-{[(1-methyl-1H-pyrrol-2- |
| | yl)carbonyl]amino}phenyl)-N-(4-methoxybenzyl)-1H-pyrazole-1-carboxamide |
| 1357 | N-(sec-butyl)-5-cyclobutyl-3-(2-hydroxy-4-{[(1-methyl-1H-pyrrol-2- |
| | yl)carbonyl]amino}phenyl)-1H-pyrazole-1-carboxamide |
| 1358 | 5-cyclobutyl-N-(2-fluorobenzyl)-3-(2-hydroxy-4-{[(1-methyl-1H-pyrrol-2- |
| | yl)carbonyl]amino}phenyl)-1H-pyrazole-1-carboxamide |
| 1359 | 5-cyclobutyl-N-(3-fluorobenzyl)-3-(2-hydroxy-4-{[(1-methyl-1H-pyrrol-2- |
| | yl)carbonyl]amino}phenyl)-1H-pyrazole-1-carboxamide |
| 1360 | 5-cyclobutyl-N-cyclopropyl-3-(2-hydroxy-4-{[(1-methyl-1H-pyrrol-2- |
| | yl)carbonyl]amino}phenyl)-1H-pyrazole-1-carboxamide |
| 1361 | 5-cyclopentyl-3-(2-hydroxy-4-{[(1-methyl-1H-pyrrol-2- |
| | yl)carbonyl]amino}phenyl)-N-isopropyl-1H-pyrazole-1-carboxamide |
| 1362 | 5-cyclopentyl-N-ethyl-3-(2-hydroxy-4-{[(1-methyl-1H-pyrrol-2- |
| | yl)carbonyl]amino}phenyl)-1H-pyrazole-1-carboxamide |
| 1363 | N-allyl-5-cyclopentyl-3-(2-hydroxy-4-{[(1-methyl-1H-pyrrol-2- |
| | yl)carbonyl]amino}phenyl)-1H-pyrazole-1-carboxamide |
| 1364 | 5-cyclopentyl-3-(2-hydroxy-4-{[(1-methyl-1H-pyrrol-2- |
| | yl)carbonyl]amino}phenyl)-N-propyl-1H-pyrazole-1-carboxamide |
| 1365 | N-benzyl-5-cyclopentyl-3-(2-hydroxy-4-{[(1-methyl-1H-pyrrol-2- |
| | yl)carbonyl]amino}phenyl)-1H-pyrazole-1-carboxamide |
| 1366 | 5-cyclopentyl-3-(2-hydroxy-4-{[(1-methyl-1H-pyrrol-2- |
| | yl)carbonyl]amino}phenyl)-N-(2-phenylethyl)-1H-pyrazole-1-carboxamide |
| 1367 | 5-cyclopentyl-3-(2-hydroxy-4-{[(1-methyl-1H-pyrrol-2- |
| | yl)carbonyl]amino}phenyl)-N-(2-methylbenzyl)-1H-pyrazole-1-carboxamide |
| 1368 | 5-cyclopentyl-3-(2-hydroxy-4-{[(1-methyl-1H-pyrrol-2- |
| | yl)carbonyl]amino}phenyl)-N-(3-methylbenzyl)-1H-pyrazole-1-carboxamide |
| 1369 | 5-cyclopentyl-3-(2-hydroxy-4-{[(1-methyl-1H-pyrrol-2- |
| | yl)carbonyl]amino}phenyl)-N-(4-methylbenzyl)-1H-pyrazole-1-carboxamide |
| 1370 | N-(2-chlorobenzyl)-5-cyclopentyl-3-(2-hydroxy-4-{[(1-methyl-1H-pyrrol-2- |
| | yl)carbonyl]amino}phenyl)-1H-pyrazole-1-carboxamide |
| 1371 | 5-cyclopentyl-N-(4-fluorobenzyl)-3-(2-hydroxy-4-{[(1-methyl-1H-pyrrol-2- |
| | yl)carbonyl]amino}phenyl)-1H-pyrazole-1-carboxamide |
| 1372 | 5-cyclopentyl-N-(3,4-dichlorobenzyl)-3-(2-hydroxy-4-{[(1-methyl-1H-pyrrol-2- |
| | yl)carbonyl]amino}phenyl)-1H-pyrazole-1-carboxamide |
| 1373 | 5-cyclopentyl-3-(2-hydroxy-4-{[(1-methyl-1H-pyrrol-2- |
| | yl)carbonyl]amino}phenyl)-N-(4-methoxybenzyl)-1H-pyrazole-1-carboxamide |
| 1374 | N-(sec-butyl)-5-cyclopentyl-3-(2-hydroxy-4-{[(1-methyl-1H-pyrrol-2- |
| | yl)carbonyl]amino}phenyl)-1H-pyrazole-1-carboxamide |
| 1375 | 5-cyclopentyl-N-(2-fluorobenzyl)-3-(2-hydroxy-4-{[(1-methyl-1H-pyrrol-2- |
| | yl)carbonyl]amino}phenyl)-1H-pyrazole-1-carboxamide |
| 1376 | 5-cyclopentyl-N-(3-fluorobenzyl)-3-(2-hydroxy-4-{[(1-methyl-1H-pyrrol-2- |
| | yl)carbonyl]amino}phenyl)-1H-pyrazole-1-carboxamide |
| 1377 | 5-cyclopentyl-N-cyclopropyl-3-(2-hydroxy-4-{[(1-methyl-1H-pyrrol-2- |
| | yl)carbonyl]amino}phenyl)-1H-pyrazole-1-carboxamide |
| 1378 | 3-{5-[(2-chlorobenzoyl)amino]-2-hydroxyphenyl}-5-cyclopropyl-N-isopropyl- |
| | 1H-pyrazole-1-carboxamide |
| 1379 | 3-{5-[(2-chlorobenzoyl)amino]-2-hydroxyphenyl}-5-cyclopropyl-N-ethyl-1H- |
| | pyrazole-1-carboxamide |
| 1380 | N-allyl-3-{5-[(2-chlorobenzoyl)amino]-2-hydroxyphenyl}-5-cyclopropyl-1H- |
| | pyrazole-1-carboxamide |
| 1381 | 3-{5-[(2-chlorobenzoyl)amino]-2-hydroxyphenyl}-5-cyclopropyl-N-propyl-1H- |
| | pyrazole-1-carboxamide |
| 1382 | N-benzyl-3-{5-[(2-chlorobenzoyl)amino]-2-hydroxyphenyl}-5-cyclopropyl-1H- |
| | pyrazole-1-carboxamide |
| 1383 | 3-{5-[(2-chlorobenzoyl)amino]-2-hydroxyphenyl}-5-cyclopropyl-N-(2- |
| | phenylethyl)-1H-pyrazole-1-carboxamide |
| 1384 | 3-{5-[(2-chlorobenzoyl)amino]-2-hydroxyphenyl}-5-cyclopropyl-N-(2- |
| | methylbenzyl)-1H-pyrazole-1-carboxamide |
| 1385 | 3-{5-[(2-chlorobenzoyl)amino]-2-hydroxyphenyl}-5-cyclopropyl-N-(3- |
| | methylbenzyl)-1H-pyrazole-1-carboxamide |
| 1386 | 3-{5-[(2-chlorobenzoyl)amino]-2-hydroxyphenyl}-5-cyclopropyl-N-(4- |
| | methylbenzyl)-1H-pyrazole-1-carboxamide |
| 1387 | 3-{5-[(2-chlorobenzoyl)amino]-2-hydroxyphenyl}-N-(2-chlorobenzyl)-5- |
| | cyclopropyl-1H-pyrazole-1-carboxamide |
| 1388 | 3-{5-[(2-chlorobenzoyl)amino]-2-hydroxyphenyl}-5-cyclopropyl-N-(4- |
| | fluorobenzyl)-1H-pyrazole-1-carboxamide |
| 1389 | 3-{5-[(2-chlorobenzoyl)amino]-2-hydroxyphenyl}-5-cyclopropyl-N-(3,4- |
| | dichlorobenzyl)-1H-pyrazole-1-carboxamide |
| 1390 | 3-{5-[(2-chlorobenzoyl)amino]-2-hydroxyphenyl}-5-cyclopropyl-N-(4- |
| | methoxybenzyl)-1H-pyrazole-1-carboxamide |
| 1391 | N-(sec-butyl)-3-{5-[(2-chlorobenzoyl)amino]-2-hydroxyphenyl}-5-cyclopropyl- |
| | 1H-pyrazole-1-carboxamide |
| 1392 | 3-{5-[(2-chlorobenzoyl)amino]-2-hydroxyphenyl}-5-cyclopropyl-N-(2- |
| | fluorobenzyl)-1H-pyrazole-1-carboxamide |
| 1393 | 3-{5-[(2-chlorobenzoyl)amino]-2-hydroxyphenyl}-5-cyclopropyl-N-(3- |
| | fluorobenzyl)-1H-pyrazole-1-carboxamide |
| 1394 | 3-{5-[(2-chlorobenzoyl)amino]-2-hydroxyphenyl}-N,5-dicyclopropyl-1H- |
| | pyrazole-1-carboxamide |
| 1395 | 3-{5-[(2-chlorobenzoyl)amino]-2-hydroxyphenyl}-5-cyclobutyl-N-isopropyl-1H- |
| | pyrazole-1-carboxamide |
| 1396 | 3-{5-[(2-chlorobenzoyl)amino]-2-hydroxyphenyl}-5-cyclobutyl-N-ethyl-1H- |
| | pyrazole-1-carboxamide |
| 1397 | N-allyl-3-{5-[(2-chlorobenzoyl)amino]-2-hydroxyphenyl}-5-cyclobutyl-1H- |
| | pyrazole-1-carboxamide |
| 1398 | 3-{5-[(2-chlorobenzoyl)amino]-2-hydroxyphenyl}-5-cyclobutyl-N-propyl-1H- |
| | pyrazole-1-carboxamide |
| 1399 | N-benzyl-3-{5-[(2-chlorobenzoyl)amino]-2-hydroxyphenyl}-5-cyclobutyl-1H- |
| | pyrazole-1-carboxamide |
| 1400 | 3-{5-[(2-chlorobenzoyl)amino]-2-hydroxyphenyl}-5-cyclobutyl-N-(2- |
| | phenylethyl)-1H-pyrazole-1-carboxamide |
| 1401 | 3-{5-[(2-chlorobenzoyl)amino]-2-hydroxyphenyl}-5-cyclobutyl-N-(2- |
| | methylbenzyl)-1H-pyrazole-1-carboxamide |
| 1402 | 3-{5-[(2-chlorobenzoyl)amino]-2-hydroxyphenyl}-5-cyclobutyl-N-(3- |
| | methylbenzyl)-1H-pyrazole-1-carboxamide |
| 1403 | 3-{5-[(2-chlorobenzoyl)amino]-2-hydroxyphenyl}-5-cyclobutyl-N-(4- |
| | methylbenzyl)-1H-pyrazole-1-carboxamide |
| 1404 | 3-{5-[(2-chlorobenzoyl)amino]-2-hydroxyphenyl}-N-(2-chlorobenzyl)-5- |
| | cyclobutyl-1H-pyrazole-1-carboxamide |
| 1405 | 3-{5-[(2-chlorobenzoyl)amino]-2-hydroxyphenyl}-5-cyclobutyl-N-(4- |
| | fluorobenzyl)-1H-pyrazole-1-carboxamide |
| 1406 | 3-{5-[(2-chlorobenzoyl)amino]-2-hydroxyphenyl}-5-cyclobutyl-N-(3,4- |
| | dichlorobenzyl)-1H-pyrazole-1-carboxamide |
| 1407 | 3-{5-[(2-chlorobenzoyl)amino]-2-hydroxyphenyl}-5-cyclobutyl-N-(4- |
| | methoxybenzyl)-1H-pyrazole-1-carboxamide |
| 1408 | N-(sec-butyl)-3-{5-[(2-chlorobenzoyl)amino]-2-hydroxyphenyl}-5-cyclobutyl- |
| | 1H-pyrazole-1-carboxamide |
| 1409 | 3-{5-[(2-chlorobenzoyl)amino]-2-hydroxyphenyl}-5-cyclobutyl-N-(2- |
| | fluorobenzyl)-1H-pyrazole-1-carboxamide |
| 1410 | 3-{5-[(2-chlorobenzoyl)amino]-2-hydroxyphenyl}-5-cyclobutyl-N-(3- |
| | fluorobenzyl)-1H-pyrazole-1-carboxamide |
| 1411 | 3-{5-[(2-chlorobenzoyl)amino]-2-hydroxyphenyl}-5-cyclobutyl-N-cyclopropyl- |
| | 1H-pyrazole-1-carboxamide |
| 1412 | 3-{5-[(2-chlorobenzoyl)amino]-2-hydroxyphenyl}-5-cyclopentyl-N-isopropyl- |
| | 1H-pyrazole-1-carboxamide |
| 1413 | 3-{5-[(2-chlorobenzoyl)amino]-2-hydroxyphenyl}-5-cyclopentyl-N-ethyl-1H- |
| | pyrazole-1-carboxamide |
| 1414 | N-allyl-3-{5-[(2-chlorobenzoyl)amino]-2-hydroxyphenyl}-5-cyclopentyl-1H- |
| | pyrazole-1-carboxamide |
| 1415 | 3-{5-[(2-chlorobenzoyl)amino]-2-hydroxyphenyl}-5-cyclopentyl-N-propyl-1H- |
| | pyrazole-1-carboxamide |
| 1416 | N-benzyl-3-{5-[(2-chlorobenzoyl)amino]-2-hydroxyphenyl}-5-cyclopentyl-1H- |
| | pyrazole-1-carboxamide |
| 1417 | 3-{5-[(2-chlorobenzoyl)amino]-2-hydroxyphenyl}-5-cyclopentyl-N-(2- |
| | phenylethyl)-1H-pyrazole-1-carboxamide |
| 1418 | 3-{5-[(2-chlorobenzoyl)amino]-2-hydroxyphenyl}-5-cyclopentyl-N-(2- |
| | methylbenzyl)-1H-pyrazole-1-carboxamide |
| 1419 | 3-{5-[(2-chlorobenzoyl)amino]-2-hydroxyphenyl}-5-cyclopentyl-N-(3- |
| | methylbenzyl)-1H-pyrazole-1-carboxamide |
| 1420 | 3-{5-[(2-chlorobenzoyl)amino]-2-hydroxyphenyl}-5-cyclopentyl-N-(4- |
| | methylbenzyl)-1H-pyrazole-1-carboxamide |
| 1421 | 3-{5-[(2-chlorobenzoyl)amino]-2-hydroxyphenyl}-N-(2-chlorobenzyl)-5- |
| | cyclopentyl-1H-pyrazole-1-carboxamide |
| 1422 | 3-{5-[(2-chlorobenzoyl)amino]-2-hydroxyphenyl}-5-cyclopentyl-N-(4- |
| | fluorobenzyl)-1H-pyrazole-1-carboxamide |
| 1423 | 3-{5-[(2-chlorobenzoyl)amino]-2-hydroxyphenyl}-5-cyclopentyl-N-(3,4- |
| | dichlorobenzyl)-1H-pyrazole-1-carboxamide |
| 1424 | 3-{5-[(2-chlorobenzoyl)amino]-2-hydroxyphenyl}-5-cyclopentyl-N-(4- |
| | methoxybenzyl)-1H-pyrazole-1-carboxamide |
| 1425 | N-(sec-butyl)-3-{5-[(2-chlorobenzoyl)amino]-2-hydroxyphenyl}-5-cyclopentyl- |
| | 1H-pyrazole-1-carboxamide |
| 1426 | 3-{5-[(2-chlorobenzoyl)amino]-2-hydroxyphenyl}-5-cyclopentyl-N-(2- |
| | fluorobenzyl)-1H-pyrazole-1-carboxamide |
| 1427 | 3-{5-[(2-chlorobenzoyl)amino]-2-hydroxyphenyl}-5-cyclopentyl-N-(3- |
| | fluorobenzyl)-1H-pyrazole-1-carboxamide |
| 1428 | 3-{5-[(2-chlorobenzoyl)amino]-2-hydroxyphenyl}-5-cyclopentyl-N-cyclopropyl- |
| | 1H-pyrazole-1-carboxamide |
| 1429 | 5-cyclopropyl-3-{5-[(2,6-dichlorobenzoyl)amino]-2-hydroxyphenyl}-N- |
| | isopropyl-1H-pyrazole-1-carboxamide |
| 1430 | 5-cyclopropyl-3-{5-[(2,6-dichlorobenzoyl)amino]-2-hydroxyphenyl}-N-ethyl-1H- |
| | pyrazole-1-carboxamide |
| 1431 | N-allyl-5-cyclopropyl-3-{5-[(2,6-dichlorobenzoyl)amino]-2-hydroxyphenyl}-1H- |
| | pyrazole-1-carboxamide |
| 1432 | 5-cyclopropyl-3-{5-[(2,6-dichlorobenzoyl)amino]-2-hydroxyphenyl}-N-propyl- |
| | 1H-pyrazole-1-carboxamide |
| 1433 | N-benzyl-5-cyclopropyl-3-{5-[(2,6-dichlorobenzoyl)amino]-2-hydroxyphenyl}- |
| | 1H-pyrazole-1-carboxamide |
| 1434 | 5-cyclopropyl-3-{5-[(2,6-dichlorobenzoyl)amino]-2-hydroxyphenyl}-N-(2- |
| | phenylethyl)-1H-pyrazole-1-carboxamide |
| 1435 | 5-cyclopropyl-3-{5-[(2,6-dichlorobenzoyl)amino]-2-hydroxyphenyl}-N-(2- |
| | methylbenzyl)-1H-pyrazole-1-carboxamide |
| 1436 | 5-cyclopropyl-3-{5-[(2,6-dichlorobenzoyl)amino]-2-hydroxyphenyl}-N-(3- |
| | methylbenzyl)-1H-pyrazole-1-carboxamide |
| 1437 | 5-cyclopropyl-3-{5-[(2,6-dichlorobenzoyl)amino]-2-hydroxyphenyl}-N-(4- |
| | methylbenzyl)-1H-pyrazole-1-carboxamide |
| 1438 | N-(2-chlorobenzyl)-5-cyclopropyl-3-{5-[(2,6-dichlorobenzoyl)amino]-2- |
| | hydroxyphenyl}-1H-pyrazole-1-carboxamide |
| 1439 | 5-cyclopropyl-3-{5-[(2,6-dichlorobenzoyl)amino]-2-hydroxyphenyl}-N-(4- |
| | fluorobenzyl)-1H-pyrazole-1-carboxamide |
| 1440 | 5-cyclopropyl-3-{5-[(2,6-dichlorobenzoyl)amino]-2-hydroxyphenyl}-N-(3,4- |
| | dichlorobenzyl)-1H-pyrazole-1-carboxamide |
| 1441 | 5-cyclopropyl-3-{5-[(2,6-dichlorobenzoyl)amino]-2-hydroxyphenyl}-N-(4- |
| | methoxybenzyl)-1H-pyrazole-1-carboxamide |
| 1442 | N-(sec-butyl)-5-cyclopropyl-3-{5-[(2,6-dichlorobenzoyl)amino]-2- |
| | hydroxyphenyl}-1H-pyrazole-1-carboxamide |
| 1443 | 5-cyclopropyl-3-{5-[(2,6-dichlorobenzoyl)amino]-2-hydroxyphenyl}-N-(2- |
| | fluorobenzyl)-1H-pyrazole-1-carboxamide |
| 1444 | 5-cyclopropyl-3-{5-[(2,6-dichlorobenzoyl)amino]-2-hydroxyphenyl}-N-(3- |
| | fluorobenzyl)-1H-pyrazole-1-carboxamide |
| 1445 | N,5-dicyclopropyl-3-{5-[(2,6-dichlorobenzoyl)amino]-2-hydroxyphenyl}-1H- |
| | pyrazole-1-carboxamide |
| 1446 | 5-cyclobutyl-3-{5-[(2,6-dichlorobenzoyl)amino]-2-hydroxyphenyl}-N-isopropyl- |
| | 1H-pyrazole-1-carboxamide |
| 1447 | 5-cyclobutyl-3-{5-[(2,6-dichlorobenzoyl)amino]-2-hydroxyphenyl}-N-ethyl-1H- |
| | pyrazole-1-carboxamide |
| 1448 | N-allyl-5-cyclobutyl-3-{5-[(2,6-dichlorobenzoyl)amino]-2-hydroxyphenyl}-1H- |
| | pyrazole-1-carboxamide |
| 1449 | 5-cyclobutyl-3-{5-[(2,6-dichlorobenzoyl)amino]-2-hydroxyphenyl}-N-propyl-1H- |
| | pyrazole-1-carboxamide |
| 1450 | N-benzyl-5-cyclobutyl-3-{5-[(2,6-dichlorobenzoyl)amino]-2-hydroxyphenyl}- |
| | 1H-pyrazole-1-carboxamide |
| 1451 | 5-cyclobutyl-3-{5-[(2,6-dichlorobenzoyl)amino]-2-hydroxyphenyl}-N-(2- |
| | phenylethyl)-1H-pyrazole-1-carboxamide |
| 1452 | 5-cyclobutyl-3-{5-[(2,6-dichlorobenzoyl)amino]-2-hydroxyphenyl}-N-(2- |
| | methylbenzyl)-1H-pyrazole-1-carboxamide |
| 1453 | 5-cyclobutyl-3-{5-[(2,6-dichlorobenzoyl)amino]-2-hydroxyphenyl}-N-(3- |
| | methylbenzyl)-1H-pyrazole-1-carboxamide |
| 1454 | 5-cyclobutyl-3-{5-[(2,6-dichlorobenzoyl)amino]-2-hydroxyphenyl}-N-(4- |
| | methylbenzyl)-1H-pyrazole-1-carboxamide |
| 1455 | N-(2-chlorobenzyl)-5-cyclobutyl-3-{5-[(2,6-dichlorobenzoyl)amino]-2- |
| | hydroxyphenyl}-1H-pyrazole-1-carboxamide |
| 1456 | 5-cyclobutyl-3-{5-[(2,6-dichlorobenzoyl)amino]-2-hydroxyphenyl}-N-(4- |
| | fluorobenzyl)-1H-pyrazole-1-carboxamide |
| 1457 | 5-cyclobutyl-3-{5-[(2,6-dichlorobenzoyl)amino]-2-hydroxyphenyl}-N-(3,4- |
| | dichlorobenzyl)-1H-pyrazole-1-carboxamide |
| 1458 | 5-cyclobutyl-3-{5-[(2,6-dichlorobenzoyl)amino]-2-hydroxyphenyl}-N-(4- |
| | methoxybenzyl)-1H-pyrazole-1-carboxamide |
| 1459 | N-(sec-butyl)-5-cyclobutyl-3-{5-[(2,6-dichlorobenzoyl)amino]-2- |
| | hydroxyphenyl}-1H-pyrazole-1-carboxamide |
| 1460 | 5-cyclobutyl-3-{5-[(2,6-dichlorobenzoyl)amino]-2-hydroxyphenyl}-N-(2- |
| | fluorobenzyl)-1H-pyrazole-1-carboxamide |
| 1461 | 5-cyclobutyl-3-{5-[(2,6-dichlorobenzoyl)amino]-2-hydroxyphenyl}-N-(3- |
| | fluorobenzyl)-1H-pyrazole-1-carboxamide |
| 1462 | 5-cyclobutyl-N-cyclopropyl-3-{5-[(2,6-dichlorobenzoyl)amino]-2- |
| | hydroxyphenyl}-1H-pyrazole-1-carboxamide |
| 1463 | 5-cyclopentyl-3-{5-[(2,6-dichlorobenzoyl)amino]-2-hydroxyphenyl}-N- |
| | isopropyl-1H-pyrazole-1-carboxamide |
| 1464 | 5-cyclopentyl-3-{5-[(2,6-dichlorobenzoyl)amino]-2-hydroxyphenyl}-N-ethyl-1H- |
| | pyrazole-1-carboxamide |
| 1465 | N-allyl-5-cyclopentyl-3-{5-[(2,6-dichlorobenzoyl)amino]-2-hydroxyphenyl}-1H- |
| | pyrazole-1-carboxamide |
| 1466 | 5-cyclopentyl-3-{5-[(2,6-dichlorobenzoyl)amino]-2-hydroxyphenyl}-N-propyl- |
| | 1H-pyrazole-1-carboxamide |
| 1467 | N-benzyl-5-cyclopentyl-3-{5-[(2,6-dichlorobenzoyl)amino]-2-hydroxyphenyl}- |
| | 1H-pyrazole-1-carboxamide |
| 1468 | 5-cyclopentyl-3-{5-[(2,6-dichlorobenzoyl)amino]-2-hydroxyphenyl}-N-(2- |
| | phenylethyl)-1H-pyrazole-1-carboxamide |
| 1469 | 5-cyclopentyl-3-{5-[(2,6-dichlorobenzoyl)amino]-2-hydroxyphenyl}-N-(2- |
| | methylbenzyl)-1H-pyrazole-1-carboxamide |
| 1470 | 5-cyclopentyl-3-{5-[(2,6-dichlorobenzoyl)amino]-2-hydroxyphenyl}-N-(3- |
| | methylbenzyl)-1H-pyrazole-1-carboxamide |
| 1471 | 5-cyclopentyl-3-{5-[(2,6-dichlorobenzoyl)amino]-2-hydroxyphenyl}-N-(4- |
| | methylbenzyl)-1H-pyrazole-1-carboxamide |
| 1472 | N-(2-chlorobenzyl)-5-cyclopentyl-3-{5-[(2,6-dichlorobenzoyl)amino]-2- |
| | hydroxyphenyl}-1H-pyrazole-1-carboxamide |
| 1473 | 5-cyclopentyl-3-{5-[(2,6-dichlorobenzoyl)amino]-2-hydroxyphenyl}-N-(4- |
| | fluorobenzyl)-1H-pyrazole-1-carboxamide |
| 1474 | 5-cyclopentyl-3-{5-[(2,6-dichlorobenzoyl)amino]-2-hydroxyphenyl}-N-(3,4- |
| | dichlorobenzyl)-1H-pyrazole-1-carboxamide |
| 1475 | 5-cyclopentyl-3-{5-[(2,6-dichlorobenzoyl)amino]-2-hydroxyphenyl}-N-(4- |
| | methoxybenzyl)-1H-pyrazole-1-carboxamide |
| 1476 | N-(sec-butyl)-5-cyclopentyl-3-{5-[(2,6-dichlorobenzoyl)amino]-2- |
| | hydroxyphenyl}-1H-pyrazole-1-carboxamide |
| 1477 | 5-cyclopentyl-3-{5-[(2,6-dichlorobenzoyl)amino]-2-hydroxyphenyl}-N-(2- |
| | fluorobenzyl)-1H-pyrazole-1-carboxamide |
| 1478 | 5-cyclopentyl-3-{5-[(2,6-dichlorobenzoyl)amino]-2-hydroxyphenyl}-N-(3- |
| | fluorobenzyl)-1H-pyrazole-1-carboxamide |
| 1479 | 5-cyclopentyl-N-cyclopropyl-3-{5-[(2,6-dichlorobenzoyl)amino]-2- |
| | hydroxyphenyl}-1H-pyrazole-1-carboxamide |
| 1480 | 5-cyclopropyl-3-{2-hydroxy-5-[(2-methoxybenzoyl)amino]phenyl}-N-isopropyl- |
| | 1H-pyrazole-1-carboxamide |
| 1481 | 5-cyclopropyl-N-ethyl-3-{2-hydroxy-5-[(2-methoxybenzoyl)amino]phenyl}-1H- |
| | pyrazole-1-carboxamide |
| 1482 | N-allyl-5-cyclopropyl-3-{2-hydroxy-5-[(2-methoxybenzoyl)amino]phenyl}-1H- |
| | pyrazole-1-carboxamide |
| 1483 | 5-cyclopropyl-3-{2-hydroxy-5-[(2-methoxybenzoyl)amino]phenyl}-N-propyl-1H- |
| | pyrazole-1-carboxamide |
| 1484 | N-benzyl-5-cyclopropyl-3-{2-hydroxy-5-[(2-methoxybenzoyl)amino]phenyl}- |
| | 1H-pyrazole-1-carboxamide |
| 1485 | 5-cyclopropyl-3-{2-hydroxy-5-[(2-methoxybenzoyl)amino]phenyl}-N-(2- |
| | phenylethyl)-1H-pyrazole-1-carboxamide |
| 1486 | 5-cyclopropyl-3-{2-hydroxy-5-[(2-methoxybenzoyl)amino]phenyl}-N-(2- |
| | methylbenzyl)-1H-pyrazole-1-carboxamide |
| 1487 | 5-cyclopropyl-3-{2-hydroxy-5-[(2-methoxybenzoyl)amino]phenyl}-N-(3- |
| | methylbenzyl)-1H-pyrazole-1-carboxamide |
| 1488 | 5-cyclopropyl-3-{2-hydroxy-5-[(2-methoxybenzoyl)amino]phenyl}-N-(4- |
| | methylbenzyl)-1H-pyrazole-1-carboxamide |
| 1489 | N-(2-chlorobenzyl)-5-cyclopropyl-3-{2-hydroxy-5-[(2- |
| | methoxybenzoyl)amino]phenyl}-1H-pyrazole-1-carboxamide |
| 1490 | 5-cyclopropyl-N-(4-fluorobenzyl)-3-{2-hydroxy-5-[(2- |
| | methoxybenzoyl)amino]phenyl}-1H-pyrazole-1-carboxamide |
| 1491 | 5-cyclopropyl-N-(3,4-dichlorobenzyl)-3-{2-hydroxy-5-[(2- |
| | methoxybenzoyl)amino]phenyl}-1H-pyrazole-1-carboxamide |
| 1492 | 5-cyclopropyl-3-{2-hydroxy-5-[(2-methoxybenzoyl)amino]phenly}-N-(4- |
| | methoxybenzyl)-1H-pyrazole-1-carboxamide |
| 1493 | N-(sec-butyl)-5-cyclopropyl-3-{2-hydroxy-5-[(2- |
| | methoxybenzoyl)amino]phenyl}-1H-pyrazole-1-carboxamide |
| 1494 | 5-cyclopropyl-N-(2-fluorobenzyl)-3-{2-hydroxy-5-[(2- |
| | methoxybenzoyl)amino]phenyl}-1H-pyrazole-1-carboxamide |
| 1495 | 5-cyclopropyl-N-(3-fluorobenzyl)-3-{2-hydroxy-5-[(2- |
| | methoxybenzoyl)amino]phenyl}-1H-pyrazole-1-carboxamide |
| 1496 | N,5-dicyclopropyl-3-{2-hydroxy-5-[(2-methoxybenzoyl)amino]phenyl}-1H- |
| | pyrazole-1-carboxamide |
| 1497 | 5-cyclobutyl-3-{2-hydroxy-5-[(2-methoxybenzoyl)amino]phenyl}-N-isopropyl- |
| | 1H-pyrazole-1-carboxamide |
| 1498 | 5-cyclobutyl-N-ethyl-3-{2-hydroxy-5-[(2-methoxybenzoyl)amino]phenyl}-1H- |
| | pyrazole-1-carboxamide |
| 1499 | N-allyl-5-cyclobutyl-3-{2-hydroxy-5-[(2-methoxybenzoyl)amino]phenyl}-1H- |
| | pyrazole-1-carboxamide |
| 1500 | 5-cyclobutyl-3-{2-hydroxy-5-[(2-methoxybenzoyl)amino]phenyl}-N-propyl-1H- |
| | pyrazole-1-carboxamide |
| 1501 | N-benzyl-5-cyclobutyl-3-{2-hydroxy-5-[(2-methoxybenzoyl)amino]phenyl}-1H- |
| | pyrazole-1-carboxamide |
| 1502 | 5-cyclobutyl-3-{2-hydroxy-5-[(2-methoxybenzoyl)amino]phenyl}-N-(2- |
| | phenylethyl)-1H-pyrazole-1-carboxamide |
| 1503 | 5-cyclobutyl-3-{2-hydroxy-5-[(2-methoxybenzoyl)amino]phenyl}-N-(2- |
| | methylbenzyl)-1H-pyrazole-1-carboxamide |
| 1504 | 5-cyclobutyl-3-{2-hydroxy-5-[(2-methoxybenzoyl)amino]phenyl}-N-(3- |
| | methylbenzyl)-1H-pyrazole-1-carboxamide |
| 1505 | 5-cyclobutyl-3-{2-hydroxy-5-[(2-methoxybenzoyl)amino]phenyl}-N-(4- |
| | methylbenzyl)-1H-pyrazole-1-carboxamide |
| 1506 | N-(2-chlorobenzyl)-5-cyclobutyl-3-{2-hydroxy-5-[(2- |
| | methoxybenzoyl)amino]phenyl}-1H-pyrazole-1-carboxamide |
| 1507 | 5-cyclobutyl-N-(4-fluorobenzyl)-3-{2-hydroxy-5-[(2- |
| | methoxybenzoyl)amino]phenyl}-1H-pyrazole-1-carboxamide |
| 1508 | 5-cyclobutyl-N-(3,4-dichlorobenzyl)-3-{2-hydroxy-5-[(2- |
| | methoxybenzoyl)amino]phenyl}-1H-pyrazole-1-carboxamide |
| 1509 | 5-cyclobutyl-3-{2-hydroxy-5-[(2-methoxybenzoyl)amino]phenyl}-N-(4- |
| | methoxybenzyl)-1H-pyrazole-1-carboxamide |
| 1510 | N-(sec-butyl)-5-cyclobutyl-3-{2-hydroxy-5-[(2-methoxybenzoyl)amino]phenyl}- |
| | 1H-pyrazole-1-carboxamide |
| 1511 | 5-cyclobutyl-N-(2-fluorobenzyl)-3-{2-hydroxy-5-[(2- |
| | methoxybenzoyl)amino]phenyl}-1H-pyrazole-1-carboxamide |
| 1512 | 5-cyclobutyl-N-(3-fluorobenzyl)-3-{2-hydroxy-5-[(2- |
| | methoxybenzoyl)amino]phenyl}-1H-pyrazole-1-carboxamide |
| 1513 | 5-cyclobutyl-N-cyclopropyl-3-{2-hydroxy-5-[(2- |
| | methoxybenzoyl)amino]phenyl}-1H-pyrazole-1-carboxamide |
| 1514 | 5-cyclopentyl-3-{2-hydroxy-5-[(2-methoxybenzoyl)amino]phenyl}-N-isopropyl- |
| | 1H-pyrazole-1-carboxamide |
| 1515 | 5-cyclopentyl-N-ethyl-3-{2-hydroxy-5-[(2-methoxybenzoyl)amino]phenyl}-1H- |
| | pyrazole-1-carboxamide |
| 1516 | N-allyl-5-cyclopentyl-3-{2-hydroxy-5-[(2-methoxybenzoyl)amino]phenyl}-1H- |
| | pyrazole-1-carboxamide |
| 1517 | 5-cyclopentyl-3-{2-hydroxy-5-[(2-methoxybenzoyl)amino]phenyl}-N-propyl-1H- |
| | pyrazole-1-carboxamide |
| 1518 | N-benzyl-5-cyclopentyl-3-{2-hydroxy-5-[(2-methoxybenzoyl)amino]phenyl}-1H- |
| | pyrazole-1-carboxamide |
| 1519 | 5-cyclopentyl-3-{2-hydroxy-5-[(2-methoxybenzoyl)amino]phenyl}-N-(2- |
| | phenylethyl)-1H-pyrazole-1-carboxamide |
| 1520 | 5-cyclopentyl-3-{2-hydroxy-5-[(2-methoxybenzoyl)amino]phenyl}-N-(2- |
| | methylbenzyl)-1H-pyrazole-1-carboxamide |
| 1521 | 5-cyclopentyl-3-{2-hydroxy-5-[(2-methoxybenzoyl)amino]phenyl}-N-(3- |
| | methylbenzyl)-1H-pyrazole-1-carboxamide |
| 1522 | 5-cyclopentyl-3-{2-hydroxy-5-[(2-methoxybenzoyl)amino]phenyl}-N-(4- |
| | methylbenzyl)-1H-pyrazole-1-carboxamide |
| 1523 | N-(2-chlorobenzyl)-5-cyclopentyl-3-{2-hydroxy-5-[(2- |
| | methoxybenzoyl)amino]phenyl}-1H-pyrazole-1-carboxamide |
| 1524 | 5-cyclopentyl-N-(4-fluorobenzyl)-3-{2-hydroxy-5-[(2- |
| | methoxybenzoyl)amino]phenyl}-1H-pyrazole-1-carboxamide |
| 1525 | 5-cyclopentyl-N-(3,4-dichlorobenzyl)-3-{2-hydroxy-5-[(2- |
| | methoxybenzoyl)amino]phenyl}-1H-pyrazole-1-carboxamide |
| 1526 | 5-cyclopentyl-3-{2-hydroxy-5-[(2-methoxybenzoyl)amino]phenyl}-N-(4- |
| | methoxybenzyl)-1H-pyrazole-1-carboxamide |
| 1527 | N-(sec-butyl)-5-cyclopentyl-3-{2-hydroxy-5-[(2- |
| | methoxybenzoyl)amino]phenyl}-1H-pyrazole-1-carboxamide |
| 1528 | 5-cyclopentyl-N-(2-fluorobenzyl)-3-{2-hydroxy-5-[(2- |
| | methoxybenzoyl)amino]phenyl}-1H-pyrazole-1-carboxamide |
| 1529 | 5-cyclopentyl-N-(3-fluorobenzyl)-3-{2-hydroxy-5-[(2- |
| | methoxybenzoyl)amino]phenyl}-1H-pyrazole-1-carboxamide |
| 1530 | 5-cyclopentyl-N-cyclopropyl-3-{2-hydroxy-5-[(2- |
| | methoxybenzoyl)amino]phenyl}-1H-pyrazole-1-carboxamide |
| 1531 | 5-cyclopropyl-3-{2-hydroxy-5-[(2-methylbenzoyl)amino]phenyl}-N-isopropyl- |
| | 1H-pyrazole-1-carboxamide |
| 1532 | 5-cyclopropyl-N-ethyl-3-{2-hydroxy-5-[(2-methylbenzoyl)amino]phenyl}-1H- |
| | pyrazole-1-carboxamide |
| 1533 | N-allyl-5-cyclopropyl-3-{2-hydroxy-5-[(2-methylbenzoyl)amino]phenyl}-1H- |
| | pyrazole-1-carboxamide |
| 1534 | 5-cyclopropyl-3-{2-hydroxy-5-[(2-methylbenzoyl)amino]phenyl}-N-propyl-1H- |
| | pyrazole-1-carboxamide |
| 1535 | N-benzyl-5-cyclopropyl-3-{2-hydroxy-5-[(2-methylbenzoyl)amino]phenyl}-1H- |
| | pyrazole-1-carboxamide |
| 1536 | 5-cyclopropyl-3-{2-hydroxy-5-[(2-methylbenzoyl)amino]phenyl}-N-(2- |
| | phenylethyl)-1H-pyrazole-1-carboxamide |
| 1537 | 5-cyclopropyl-3-{2-hydroxy-5-[(2-methylbenzoyl)amino]phenyl}-N-(2- |
| | methylbenzyl)-1H-pyrazole-1-carboxamide |
| 1538 | 5-cyclopropyl-3-{2-hydroxy-5-[(2-methylbenzoyl)amino]phenyl}-N-(3- |
| | methylbenzyl)-1H-pyrazole-1-carboxamide |
| 1539 | 5-cyclopropyl-3-{2-hydroxy-5-[(2-methylbenzoyl)amino]phenyl}-N-(4- |
| | methylbenzyl)-1H-pyrazole-1-carboxamide |
| 1540 | N-(2-chlorobenzyl)-5-cyclopropyl-3-{2-hydroxy-5-[(2- |
| | methylbenzoyl)amino]phenyl}-1H-pyrazole-1-carboxamide |
| 1541 | 5-cyclopropyl-N-(4-fluorobenzyl)-3-{2-hydroxy-5-[(2- |
| | methylbenzoyl)amino]phenyl}-1H-pyrazole-1-carboxamide |
| 1542 | 5-cyclopropyl-N-(3,4-dichlorobenzyl)-3-{2-hydroxy-5-[(2- |
| | methylbenzoyl)amino]phenyl}-1H-pyrazole-1-carboxamide |
| 1543 | 5-cyclopropyl-3-{2-hydroxy-5-[(2-methylbenzoyl)amino]phenyl}-N-(4- |
| | methoxybenzyl)-1H-pyrazole-1-carboxamide |
| 1544 | N-(sec-butyl)-5-cyclopropyl-3-{2-hydroxy-5-[(2-methylbenzoyl)amino]phenyl}- |
| | 1H-pyrazole-1-carboxamide |
| 1545 | 5-cyclopropyl-N-(2-fluorobenzyl)-3-{2-hydroxy-5-[(2- |
| | methylbenzoyl)amino]phenyl}-1H-pyrazole-1-carboxamide |
| 1546 | 5-cyclobutyl-3-{2-hydroxy-5-[(2-methylbenzoyl)amino]phenyl}-N-isopropyl-1H- |
| | pyrazole-1-carboxamide |
| 1547 | N,5-dicyclopropyl-3-{2-hydroxy-5-[(2-methylbenzoyl)amino]phenyl}-1H- |
| | pyrazole-1-carboxamide |
| 1548 | 5-cyclobutyl-3-{2-hydroxy-5-[(2-methylbenzoyl)amino]phenyl}-N-isopropyl-1H- |
| | pyrazole-1-carboxamide |
| 1549 | 5-cyclobutyl-N-ethyl-3-{2-hydroxy-5-[(2-methylbenzoyl)amino]phenyl}-1H- |
| | pyrazole-1-carboxamide |
| 1550 | N-allyl-5-cyclobutyl-3-{2-hydroxy-5-[(2-methylbenzoyl)amino]phenyl}-1H- |
| | pyrazole-1-carboxamide |
| 1551 | 5-cyclobutyl-3-{2-hydroxy-5-[(2-methylbenzoyl)amino]phenyl}-N-propyl-1H- |
| | pyrazole-1-carboxamide |
| 1552 | N-benzyl-5-cyclobutyl-3-{2-hydroxy-5-[(2-methylbenzoyl)amino]phenyl}-1H- |
| | pyrazole-1-carboxamide |
| 1553 | 5-cyclobutyl-3-{2-hydroxy-5-[(2-methylbenzoyl)amino]phenyl}-N-(2- |
| | phenylethyl)-1H-pyrazole-1-carboxamide |
| 1554 | 5-cyclobutyl-3-{2-hydroxy-5-[(2-methylbenzoyl)amino]phenyl}-N-(2- |
| | methylbenzyl)-1H-pyrazole-1-carboxamide |
| 1555 | 5-cyclobutyl-3-{2-hydroxy-5-[(2-methylbenzoyl)amino]phenyl}-N-(3- |
| | methylbenzyl)-1H-pyrazole-1-carboxamide |
| 1556 | 5-cyclobutyl-3-{2-hydroxy-5-[(2-methylbenzoyl)amino]phenyl}-N-(4- |
| | methylbenzyl)-1H-pyrazole-1-carboxamide |
| 1557 | N-(2-chlorobenzyl)-5-cyclobutyl-3-{2-hydroxy-5-[(2- |
| | methylbenzoyl)amino]phenyl}-1H-pyrazole-1-carboxamide |
| 1558 | 5-cyclobutyl-N-(4-fluorobenzyl)-3-{2-hydroxy-5-[(2- |
| | methylbenzoyl)amino]phenyl}-1H-pyrazole-1-carboxamide |
| 1559 | 5-cyclobutyl-N-(3,4-dichlorobenzyl)-3-{2-hydroxy-5-[(2- |
| | methylbenzoyl)amino]phenyl}-1H-pyrazole-1-carboxamide |
| 1560 | 5-cyclobutyl-3-{2-hydroxy-5-[(2-methylbenzoyl)amino]phenyl}-N-(4- |
| | methoxybenzyl)-1H-pyrazole-1-carboxamide |
| 1561 | N-(sec-butyl)-5-cyclobutyl-3-{2-hydroxy-5-[(2-methylbenzoyl)amino]phenyl}- |
| | 1H-pyrazole-1-carboxamide |
| 1562 | 5-cyclobutyl-N-(2-fluorobenzyl)-3-{2-hydroxy-5-[(2- |
| | methylbenzoyl)amino]phenyl}-1H-pyrazole-1-carboxamide |
| 1563 | 5-cyclopentyl-3-{2-hydroxy-5-[(2-methylbenzoyl)amino]phenyl}-N-isopropyl- |
| | 1H-pyrazole-1-carboxamide |
| 1564 | 5-cyclobutyl-N-cyclopropyl-3-{2-hydroxy-5-[(2-methylbenzoyl)amino]phenyl}- |
| | 1H-pyrazole-1-carboxamide |
| 1565 | 5-cyclopentyl-3-{2-hydroxy-5-[(2-methylbenzoyl)amino]phenyl}-N-isopropyl- |
| | 1H-pyrazole-1-carboxamide |
| 1566 | 5-cyclopentyl-N-ethyl-3-{2-hydroxy-5-[(2-methylbenzoyl)amino]phenyl}-1H- |
| | pyrazole-1-carboxamide |
| 1567 | N-allyl-5-cyclopentyl-3-{2-hydroxy-5-[(2-methylbenzoyl)amino]phenyl}-1H- |
| | pyrazole-1-carboxamide |
| 1568 | 5-cyclopentyl-3-{2-hydroxy-5-[(2-methylbenzoyl)amino]phenyl}-N-propyl-1H- |
| | pyrazole-1-carboxamide |
| 1569 | N-benzyl-5-cyclopentyl-3-{2-hydroxy-5-[(2-methylbenzoyl)amino]phenyl}-1H- |
| | pyrazole-1-carboxamide |
| 1570 | 5-cyclopentyl-3-{2-hydroxy-5-[(2-methylbenzoyl)amino]phenyl}-N-(2- |
| | phenylethyl)-1H-pyrazole-1-carboxamide |
| 1571 | 5-cyclopentyl-3-{2-hydroxy-5-[(2-methylbenzoyl)amino]phenyl}-N-(2- |
| | methylbenzyl)-1H-pyrazole-1-carboxamide |
| 1572 | 5-cyclopentyl-3-{2-hydroxy-5-[(2-methylbenzoyl)amino]phenyl}-N-(3- |
| | methylbenzyl)-1H-pyrazole-1-carboxamide |
| 1573 | 5-cyclopentyl-3-{2-hydroxy-5-[(2-methylbenzoyl)amino]phenyl}-N-(4- |
| | methylbenzyl)-1H-pyrazole-1-carboxamide |
| 1574 | N-(2-chlorobenzyl)-5-cyclopentyl-3-{2-hydroxy-5-[(2- |
| | methylbenzoyl)amino]phenyl}-1H-pyrazole-1-carboxamide |
| 1575 | 5-cyclopentyl-N-(4-fluorobenzyl)-3-{2-hydroxy-5-[(2- |
| | methylbenzoyl)amino]phenyl}-1H-pyrazole-1-carboxamide |
| 1576 | 5-cyclopentyl-N-(3,4-dichlorobenzyl)-3-{2-hydroxy-5-[(2- |
| | methylbenzoyl)amino]phenyl}-1H-pyrazole-1-carboxamide |
| 1577 | 5-cyclopentyl-3-{2-hydroxy-5-[(2-methylbenzoyl)amino]phenyl}-N-(4- |
| | methoxybenzyl)-1H-pyrazole-1-carboxamide |
| 1578 | N-(sec-butyl)-5-cyclopentyl-3-{2-hydroxy-5-[(2-methylbenzoyl)amino]phenyl}- |
| | 1H-pyrazole-1-carboxamide |
| 1579 | 5-cyclopentyl-N-(2-fluorobenzyl)-3-{2-hydroxy-5-[(2- |
| | methylbenzoyl)amino]phenyl}-1H-pyrazole-1-carboxamide |
| 1580 | 5-cyclopentyl-N-(3-fluorobenzyl)-3-{2-hydroxy-5-[(2- |
| | methylbenzoyl)amino]phenyl}-1H-pyrazole-1-carboxamide |
| 1581 | 5-cyclopentyl-N-cyclopropyl-3-{2-hydroxy-5-[(2-methylbenzoyl)amino]phenyl}- |
| | 1H-pyrazole-1-carboxamide |
| 1582 | 5-cyclopropyl-3-(2-hydroxy-5-{[3-(trifluoromethyl)benzoyl]amino}phenyl)-N- |
| | isopropyl-1H-pyrazole-1-carboxamide |
| 1583 | 5-cyclopropyl-N-ethyl-3-(2-hydroxy-5-{[3- |
| | (trifluoromethyl)benzoyl]amino}phenyl)-1H-pyrazole-1-carboxamide |
| 1584 | N-allyl-5-cyclopropyl-3-(2-hydroxy-5-{[3- |
| | (trifluoromethyl)benzoyl]amino}phenyl)-1H-pyrazole-1-carboxamide |
| 1585 | 5-cyclopropyl-3-(2-hydroxy-5-{[3-(trifluoromethyl)benzoyl]amino}phenyl)-N- |
| | propyl-1H-pyrazole-1-carboxamide |
| 1586 | N-benzyl-5-cyclopropyl-3-(2-hydroxy-5-{[3- |
| | (trifluoromethyl)benzoyl]amino}phenyl)-1H-pyrazole-1-carboxamide |
| 1587 | 5-cyclopropyl-3-(2-hydroxy-5-{[3-(trifluoromethyl)benzoyl]amino}phenyl)-N-(2- |
| | phenylethyl)-1H-pyrazole-1-carboxamide |
| 1588 | 5-cyclopropyl-3-(2-hydroxy-5-{[3-(trifluoromethyl)benzoyl]amino}phenyl)-N-(2- |
| | methylbenzyl)-1H-pyrazole-1-carboxamide |
| 1589 | 5-cyclopropyl-3-(2-hydroxy-5-{[3-(trifluoromethyl)benzoyl]amino}phenyl)-N-(3- |
| | methylbenzyl)-1H-pyrazole-1-carboxamide |
| 1590 | 5-cyclopropyl-3-(2-hydroxy-5-{[3-(trifluoromethyl)benzoyl]amino}phenyl)-N-(4- |
| | methylbenzyl)-1H-pyrazole-1-carboxamide |
| 1591 | N-(2-chlorobenzyl)-5-cyclopropyl-3-(2-hydroxy-5-{[3- |
| | (trifluoromethyl)benzoyl]amino}phenyl)-1H-pyrazole-1-carboxamide |
| 1592 | 5-cyclopropyl-N-(4-fluorobenzyl)-3-(2-hydroxy-5-{[3- |
| | (trifluoromethyl)benzoyl]amino}phenyl)-1H-pyrazole-1-carboxamide |
| 1593 | 5-cyclopropyl-N-(3,4-dichlorobenzyl)-3-(2-hydroxy-5-{[3- |
| | (trifluoromethyl)benzoyl]amino}phenyl)-1H-pyrazole-1-carboxamide |
| 1594 | 5-cyclopropyl-3-(2-hydroxy-5-{[3-(trifluoromethyl)benzoyl]amino}phenyl)-N-(4- |
| | methoxybenzyl)-1H-pyrazole-1-carboxamide |
| 1595 | N-(sec-butyl)-5-cyclopropyl-3-(2-hydroxy-5-{[3- |
| | (trifluoromethyl)benzoyl]amino}phenyl)-1H-pyrazole-1-carboxamide |
| 1596 | 5-cyclopropyl-N-(2-fluorobenzyl)-3-(2-hydroxy-5-{[3- |
| | (trifluoromethyl)benzoyl]amino}phenyl)-1H-pyrazole-1-carboxamide |
| 1597 | 5-cyclopropyl-N-(3-fluorobenzyl)-3-(2-hydroxy-5-{[3- |
| | (trifluoromethyl)benzoyl]amino}phenyl)-1H-pyrazole-1-carboxamide |
| 1598 | N,5-dicyclopropyl-3-(2-hydroxy-5-{[3-(trifluoromethyl)benzoyl]amino}phenyl)- |
| | 1H-pyrazole-1-carboxamide |
| 1599 | 5-cyclobutyl-3-(2-hydroxy-5-{[3-(trifluoromethyl)benzoyl]amino}phenyl)-N- |
| | isopropyl-1H-pyrazole-1-carboxamide |
| 1600 | 5-cyclobutyl-N-ethyl-3-(2-hydroxy-5-{[3- |
| | (trifluoromethyl)benzoyl]amino}phenyl)-1H-pyrazole-1-carboxamide |
| 1601 | N-allyl-5-cyclobutyl-3-(2-hydroxy-5-{[3- |
| | (trifluoromethyl)benzoyl]amino}phenyl)-1H-pyrazole-1-carboxamide |
| 1602 | 5-cyclobutyl-3-(2-hydroxy-5-{[3-(trifluoromethyl)benzoyl]amino}phenyl)-N- |
| | propyl-1H-pyrazole-1-carboxamide |
| 1603 | N-benzyl-5-cyclobutyl-3-(2-hydroxy-5-{[3- |
| | (trifluoromethyl)benzoyl]amino}phenyl)-1H-pyrazole-1-carboxamide |
| 1604 | 5-cyclobutyl-3-(2-hydroxy-5-{[3-(trifluoromethyl)benzoyl]amino}phenyl)-N-(2- |
| | phenylethyl)-1H-pyrazole-1-carboxamide |
| 1605 | 5-cyclobutyl-3-(2-hydroxy-5-{[3-(trifluoromethyl)benzoyl]amino}phenyl)-N-(2- |
| | methylbenzyl)-1H-pyrazole-1-carboxamide |
| 1606 | 5-cyclobutyl-3-(2-hydroxy-5-{[3-(trifluoromethyl)benzoyl]amino}phenyl)-N-(3- |
| | methylbenzyl)-1H-pyrazole-1-carboxamide |
| 1607 | 5-cyclobutyl-3-(2-hydroxy-5-{[3-(trifluoromethyl)benzoyl]amino}phenyl)-N-(4- |
| | methylbenzyl)-1H-pyrazole-1-carboxamide |
| 1608 | N-(2-chlorobenzyl)-5-cyclobutyl-3-(2-hydroxy-5-{[3- |
| | (trifluoromethyl)benzoyl]amino}phenyl)-1H-pyrazole-1-carboxamide |
| 1609 | 5-cyclobutyl-N-(4-fluorobenzyl)-3-(2-hydroxy-5-{[3- |
| | (trifluoromethyl)benzoyl]amino}phenyl)-1H-pyrazole-1-carboxamide |
| 1610 | 5-cyclobutyl-N-(3,4-dichlorobenzyl)-3-(2-hydroxy-5-{[3- |
| | (trifluoromethyl)benzoyl]amino}phenyl)-1H-pyrazole-1-carboxamide |
| 1611 | 5-cyclobutyl-3-(2-hydroxy-5-{[3-(trifluoromethyl)benzoyl]amino}phenyl)-N-(4- |
| | methoxybenzyl)-1H-pyrazole-1-carboxamide |
| 1612 | N-(sec-butyl)-5-cyclobutyl-3-(2-hydroxy-5-{[3- |
| | (trifluoromethyl)benzoyl]amino}phenyl)-1H-pyrazole-1-carboxamide |
| 1613 | 5-cyclobutyl-N-(2-fluorobenzyl)-3-(2-hydroxy-5-{[3- |
| | (trifluoromethyl)benzoyl]amino}phenyl)-1H-pyrazole-1-carboxamide |
| 1614 | 5-cyclobutyl-N-(3-fluorobenzyl)-3-(2-hydroxy-5-{[3- |
| | (trifluoromethyl)benzoyl]amino}phenyl)-1H-pyrazole-1-carboxamide |
| 1615 | 5-cyclobutyl-N-cyclopropyl-3-(2-hydroxy-5-{[3- |
| | (trifluoromethyl)benzoyl]amino}phenyl)-1H-pyrazole-1-carboxamide |
| 1616 | 5-cyclopentyl-3-(2-hydroxy-5-{[3-(trifluoromethyl)benzoyl]amino}phenyl)-N- |
| | isopropyl-1H-pyrazole-1-carboxamide |
| 1617 | 5-cyclopentyl-N-ethyl-3-(2-hydroxy-5-{[3- |
| | (trifluoromethyl)benzoyl]amino}phenyl)-1H-pyrazole-1-carboxamide |
| 1618 | N-allyl-5-cyclopentyl-3-(2-hydroxy-5-{[3- |
| | (trifluoromethyl)benzoyl]amino}phenyl)-1H-pyrazole-1-carboxamide |
| 1619 | 5-cyclopentyl-3-(2-hydroxy-5-{[3-(trifluoromethyl)benzoyl]amino}phenyl)-N- |
| | propyl-1H-pyrazole-1-carboxamide |
| 1620 | N-benzyl-5-cyclopentyl-3-(2-hydroxy-5-{[3- |
| | (trifluoromethyl)benzoyl]amino}phenyl)-1H-pyrazole-1-carboxamide |
| 1621 | 5-cyclopentyl-3-(2-hydroxy-5-{[3-(trifluoromethyl)benzoyl]amino}phenyl)-N-(2- |
| | phenylethyl)-1H-pyrazole-1-carboxamide |
| 1622 | 5-cyclopentyl-3-(2-hydroxy-5-{[3-(trifluoromethyl)benzoyl]amino}phenyl)-N-(2- |
| | methylbenzyl)-1H-pyrazole-1-carboxamide |
| 1623 | 5-cyclopentyl-3-(2-hydroxy-5-{[3-(trifluoromethyl)benzoyl]amino}phenyl)-N-(3- |
| | methylbenzyl)-1H-pyrazole-1-carboxamide |
| 1624 | 5-cyclopentyl-3-(2-hydroxy-5-{[3-(trifluoromethyl)benzoyl]amino}phenyl)-N-(4- |
| | methylbenzyl)-1H-pyrazole-1-carboxamide |
| 1625 | N-(2-chlorobenzyl)-5-cyclopentyl-3-(2-hydroxy-5-{[3- |
| | (trifluoromethyl)benzoyl]amino}phenyl)-1H-pyrazole-1-carboxamide |
| 1626 | 5-cyclopentyl-N-(4-fluorobenzyl)-3-(2-hydroxy-5-{[3- |
| | (trifluoromethyl)benzoyl]amino}phenyl)-1H-pyrazole-1-carboxamide |
| 1627 | 5-cyclopentyl-N-(3,4-dichlorobenzyl)-3-(2-hydroxy-5-{[3- |
| | (trifluoromethyl)benzoyl]amino}phenyl)-1H-pyrazole-1-carboxamide |
| 1628 | 5-cyclopentyl-3-(2-hydroxy-5-{[3-(trifluoromethyl)benzoyl]amino}phenyl)-N-(4- |
| | methoxybenzyl)-1H-pyrazole-1-carboxamide |
| 1629 | N-(sec-butyl)-5-cyclopentyl-3-(2-hydroxy-5-{[3- |
| | (trifluoromethyl)benzoyl]amino}phenyl)-1H-pyrazole-1-carboxamide |
| 1630 | 5-cyclopentyl-N-(2-fluorobenzyl)-3-(2-hydroxy-5-{[3- |
| | (trifluoromethyl)benzoyl]amino}phenyl)-1H-pyrazole-1-carboxamide |
| 1631 | 5-cyclopentyl-N-(3-fluorobenzyl)-3-(2-hydroxy-5-{[3- |
| | (trifluoromethyl)benzoyl]amino}phenyl)-1H-pyrazole-1-carboxamide |
| 1632 | 5-cyclopentyl-N-cyclopropyl-3-(2-hydroxy-5-{[3- |
| | (trifluoromethyl)benzoyl]amino}phenyl)-1H-pyrazole-1-carboxamide |
| 1633 | 3-{5-[(4-chlorobenzoyl)amino]-2-hydroxyphenyl}-5-cyclopropyl-N-isopropyl- |
| | 1H-pyrazole-1-carboxamide |
| 1634 | 3-{5-[(4-chlorobenzoyl)amino]-2-hydroxyphenyl}-5-cyclopropyl-N-ethyl-1H- |
| | pyrazole-1-carboxamide |
| 1635 | N-allyl-3-{5-[(4-chlorobenzoyl)amino]-2-hydroxyphenyl}-5-cyclopropyl-1H- |
| | pyrazole-1-carboxamide |
| 1636 | 3-{5-[(4-chlorobenzoyl)amino]-2-hydroxyphenyl}-5-cyclopropyl-N-propyl-1H- |
| | pyrazole-1-carboxamide |
| 1637 | N-benzyl-3-{5-[(4-chlorobenzoyl)amino]-2-hydroxyphenyl}-5-cyclopropyl-1H- |
| | pyrazole-1-carboxamide |
| 1638 | 3-{5-[(4-chlorobenzoyl)amino]-2-hydroxyphenyl}-5-cyclopropyl-N-(2- |
| | phenylethyl)-1H-pyrazole-1-carboxamide |
| 1639 | 3-{5-[(4-chlorobenzoyl)amino]-2-hydroxyphenyl}-5-cyclopropyl-N-(2- |
| | methylbenzyl)-1H-pyrazole-1-carboxamide |
| 1640 | 3-{5-[(4-chlorobenzoyl)amino]-2-hydroxyphenyl}-5-cyclopropyl-N-(3- |
| | methylbenzyl)-1H-pyrazole-1-carboxamide |
| 1641 | 3-{5-[(4-chlorobenzoyl)amino]-2-hydroxyphenyl}-5-cyclopropyl-N-(4- |
| | methylbenzyl)-1H-pyrazole-1-carboxamide |
| 1642 | 3-{5-[(4-chlorobenzoyl)amino]-2-hydroxyphenyl}-N-(2-chlorobenzyl)-5- |
| | cyclopropyl-1H-pyrazole-1-carboxamide |
| 1643 | 3-{5-[(4-chlorobenzoyl)amino]-2-hydroxyphenyl}-5-cyclopropyl-N-(4- |
| | fluorobenzyl)-1H-pyrazole-1-carboxamide |
| 1644 | 3-{5-[(4-chlorobenzoyl)amino]-2-hydroxyphenyl}-5-cyclopropyl-N-(3,4- |
| | dichlorobenzyl)-1H-pyrazole-1-carboxamide |
| 1645 | 3-{5-[(4-chlorobenzoyl)amino]-2-hydroxyphenyl}-5-cyclopropyl-N-(4- |
| | methoxybenzyl)-1H-pyrazole-1-carboxamide |
| 1646 | N-(sec-butyl)-3-{5-[(4-chlorobenzoyl)amino]-2-hydroxyphenyl}-5-cyclopropyl- |
| | 1H-pyrazole-1-carboxamide |
| 1647 | 3-{5-[(4-chlorobenzoyl)amino]-2-hydroxyphenyl}-5-cyclopropyl-N-(2- |
| | fluorobenzyl)-1H-pyrazole-1-carboxamide |
| 1648 | 3-{5-[(4-chlorobenzoyl)amino]-2-hydroxyphenyl}-5-cyclopropyl-N-(3- |
| | fluorobenzyl)-1H-pyrazole-1-carboxamide |
| 1649 | 3-{5-[(4-chlorobenzoyl)amino]-2-hydroxyphenyl}-N,5-dicyclopropyl-1H- |
| | pyrazole-1-carboxamide |
| 1650 | 3-{5-[(4-chlorobenzoyl)amino]-2-hydroxyphenyl}-5-cyclobutyl-N-isopropyl-1H- |
| | pyrazole-1-carboxamide |
| 1651 | 3-{5-[(4-chlorobenzoyl)amino]-2-hydroxyphenyl}-5-cyclobutyl-N-ethyl-1H- |
| | pyrazole-1-carboxamide |
| 1652 | N-allyl-3-{5-[(4-chlorobenzoyl)amino]-2-hydroxyphenyl}-5-cyclobutyl-1H- |
| | pyrazole-1-carboxamide |
| 1653 | 3-{5-[(4-chlorobenzoyl)amino]-2-hydroxyphenyl}-5-cyclobutyl-N-propyl-1H- |
| | pyrazole-1-carboxamide |
| 1654 | N-benzyl-3-{5-[(4-chlorobenzoyl)amino]-2-hydroxyphenyl}-5-cyclobutyl-1H- |
| | pyrazole-1-carboxamide |
| 1655 | 3-{5-[(4-chlorobenzoyl)amino]-2-hydroxyphenyl}-5-cyclobutyl-N-(2- |
| | phenylethyl)-1H-pyrazole-1-carboxamide |
| 1656 | 3-{5-[(4-chlorobenzoyl)amino]-2-hydroxyphenyl}-5-cyclobutyl-N-(2- |
| | methylbenzyl)-1H-pyrazole-1-carboxamide |
| 1657 | 3-{5-[(4-chlorobenzoyl)amino]-2-hydroxyphenyl}-5-cyclobutyl-N-(3- |
| | methylbenzyl)-1H-pyrazole-1-carboxamide |
| 1658 | 3-{5-[(4-chlorobenzoyl)amino]-2-hydroxyphenyl}-5-cyclobutyl-N-(4- |
| | methylbenzyl)-1H-pyrazole-1-carboxamide |
| 1659 | 3-{5-[(4-chlorobenzoyl)amino]-2-hydroxyphenyl}-N-(2-chlorobenzyl)-5- |
| | cyclobutyl-1H-pyrazole-1-carboxamide |
| 1660 | 3-{5-[(4-chlorobenzoyl)amino]-2-hydroxyphenyl}-5-cyclobutyl-N-(4- |
| | fluorobenzyl)-1H-pyrazole-1-carboxamide |
| 1661 | 3-{5-[(4-chlorobenzoyl)amino]-2-hydroxyphenyl}-5-cyclobutyl-N-(3,4- |
| | dichlorobenzyl)-1H-pyrazole-1-carboxamide |
| 1662 | 3-{5-[(4-chlorobenzoyl)amino]-2-hydroxyphenyl}-5-cyclobutyl-N-(4- |
| | methoxybenzyl)-1H-pyrazole-1-carboxamide |
| 1663 | N-(sec-butyl)-3-{5-[(4-chlorobenzoyl)amino]-2-hydroxyphenyl}-5-cyclobutyl- |
| | 1H-pyrazole-1-carboxamide |
| 1664 | 3-{5-[(4-chlorobenzoyl)amino]-2-hydroxyphenyl}-5-cyclobutyl-N-(2- |
| | fluorobenzyl)-1H-pyrazole-1-carboxamide |
| 1665 | 3-{5-[(4-chlorobenzoyl)amino]-2-hydroxyphenyl}-5-cyclobutyl-N-(3- |
| | fluorobenzyl)-1H-pyrazole-1-carboxamide |
| 1666 | 3-{5-[(4-chlorobenzoyl)amino]-2-hydroxyphenyl}-5-cyclobutyl-N-cyclopropyl- |
| | 1H-pyrazole-1-carboxamide |
| 1667 | 3-{5-[(4-chlorobenzoyl)amino]-2-hydroxyphenyl}-5-cyclopentyl-N-isopropyl- |
| | 1H-pyrazole-1-carboxamide |
| 1668 | 3-{5-[(4-chlorobenzoyl)amino]-2-hydroxyphenyl}-5-cyclopentyl-N-ethyl-1H- |
| | pyrazole-1-carboxamide |
| 1669 | N-allyl-3-{5-[(4-chlorobenzoyl)amino]-2-hydroxyphenyl}-5-cyclopentyl-1H- |
| | pyrazole-1-carboxamide |
| 1670 | 3-{5-[(4-chlorobenzoyl)amino]-2-hydroxyphenyl}-5-cyclopentyl-N-propyl-1H- |
| | pyrazole-1-carboxamide |
| 1671 | N-benzyl-3-{5-[(4-chlorobenzoyl)amino]-2-hydroxyphenyl}-5-cyclopentyl-1H- |
| | pyrazole-1-carboxamide |
| 1672 | 3-{5-[(4-chlorobenzoyl)amino]-2-hydroxyphenyl}-5-cyclopentyl-N-(2- |
| | phenylethyl)-1H-pyrazole-1-carboxamide |
| 1673 | 3-{5-[(4-chlorobenzoyl)amino]-2-hydroxyphenyl}-5-cyclopentyl-N-(2- |
| | methylbenzyl)-1H-pyrazole-1-carboxamide |
| 1674 | 3-{5-[(4-chlorobenzoyl)amino]-2-hydroxyphenyl}-5-cyclopentyl-N-(3- |
| | methylbenzyl)-1H-pyrazole-1-carboxamide |
| 1675 | 3-{5-[(4-chlorobenzoyl)amino]-2-hydroxyphenyl}-5-cyclopentyl-N-(4- |
| | methylbenzyl)-1H-pyrazole-1-carboxamide |
| 1676 | 3-{5-[(4-chlorobenzoyl)amino]-2-hydroxyphenyl}-N-(2-chlorobenzyl)-5- |
| | cyclopentyl-1H-pyrazole-1-carboxamide |
| 1677 | 3-{5-[(4-chlorobenzoyl)amino]-2-hydroxyphenyl}-5-cyclopentyl-N-(4- |
| | fluorobenzyl)-1H-pyrazole-1-carboxamide |
| 1678 | 3-{5-[(4-chlorobenzoyl)amino]-2-hydroxyphenyl}-5-cyclopentyl-N-(3,4- |
| | dichlorobenzyl)-1H-pyrazole-1-carboxamide |
| 1679 | 3-{5-[(4-chlorobenzoyl)amino]-2-hydroxyphenyl}-5-cyclopentyl-N-(4- |
| | methoxybenzyl)-1H-pyrazole-1-carboxamide |
| 1680 | N-(sec-butyl)-3-{5-[(4-chlorobenzoyl)amino]-2-hydroxyphenyl}-5-cyclopentyl- |
| | 1H-pyrazole-1-carboxamide |
| 1681 | 3-{5-[(4-chlorobenzoyl)amino]-2-hydroxyphenyl}-5-cyclopentyl-N-(2- |
| | fluorobenzyl)-1H-pyrazole-1-carboxamide |
| 1682 | 3-{5-[(4-chlorobenzoyl)amino]-2-hydroxyphenyl}-5-cyclopentyl-N-(3- |
| | fluorobenzyl)-1H-pyrazole-1-carboxamide |
| 1683 | 3-{5-[(4-chlorobenzoyl)amino]-2-hydroxyphenyl}-5-cyclopentyl-N-cyclopropyl- |
| | 1H-pyrazole-1-carboxamide |
| 1684 | 5-cyclopropyl-3-{5-[(4-ethoxybenzoyl)amino]-2-hydroxyphenyl}-N-isopropyl- |
| | 1H-pyrazole-1-carboxamide |
| 1685 | 5-cyclopropyl-3-{5-[(4-ethoxybenzoyl)amino]-2-hydroxyphenyl}-N-ethyl-1H- |
| | pyrazole-1-carboxamide |
| 1686 | N-allyl-5-cyclopropyl-3-{5-[(4-ethoxybenzoyl)amino]-2-hydroxyphenyl}-1H- |
| | pyrazole-1-carboxamide |
| 1687 | 5-cyclopropyl-3-{5-[(4-ethoxybenzoyl)amino]-2-hydroxyphenyl}-N-propyl-1H- |
| | pyrazole-1-carboxamide |
| 1688 | N-benzyl-5-cyclopropyl-3-{5-[(4-ethoxybenzoyl)amino]-2-hydroxyphenyl}-1H- |
| | pyrazole-1-carboxamide |
| 1689 | 5-cyclopropyl-3-{5-[(4-ethoxybenzoyl)amino]-2-hydroxyphenyl}-N-(2- |
| | phenylethyl)-1H-pyrazole-1-carboxamide |
| 1690 | 5-cyclopropyl-3-{5-[(4-ethoxybenzoyl)amino]-2-hydroxyphenyl}-N-(2- |
| | methylbenzyl)-1H-pyrazole-1-carboxamide |
| 1691 | 5-cyclopropyl-3-{5-[(4-ethoxybenzoyl)amino]-2-hydroxyphenyl}-N-(3- |
| | methylbenzyl)-1H-pyrazole-1-carboxamide |
| 1692 | 5-cyclopropyl-3-{5-[(4-ethoxybenzoyl)amino]-2-hydroxyphenyl}-N-(4- |
| | methylbenzyl)-1H-pyrazole-1-carboxamide |
| 1693 | N-(2-chlorobenzyl)-5-cyclopropyl-3-{5-[(4-ethoxybenzoyl)amino]-2- |
| | hydroxyphenyl}-1H-pyrazole-1-carboxamide |
| 1694 | 5-cyclopropyl-3-{5-[(4-ethoxybenzoyl)amino]-2-hydroxyphenyl}-N-(4- |
| | fluorobenzyl)-1H-pyrazole-1-carboxamide |
| 1695 | 5-cyclopropyl-N-(3,4-dichlorobenzyl)-3-{5-[(4-ethoxybenzoyl)amino]-2- |
| | hydroxyphenyl}-1H-pyrazole-1-carboxamide |
| 1696 | 5-cyclopropyl-3-{5-[(4-ethoxybenzoyl)amino]-2-hydroxyphenyl}-N-(4- |
| | methoxybenzyl)-1H-pyrazole-1-carboxamide |
| 1697 | N-(sec-butyl)-5-cyclopropyl-3-{5-[(4-ethoxybenzoyl)amino]-2-hydroxyphenyl}- |
| | 1H-pyrazole-1-carboxamide |
| 1698 | 5-cyclopropyl-3-{5-[(4-ethoxybenzoyl)amino]-2-hydroxyphenyl}-N-(2- |
| | fluorobenzyl)-1H-pyrazole-1-carboxamide |
| 1699 | 5-cyclopropyl-3-{5-[(4-ethoxybenzoyl)amino]-2-hydroxyphenyl}-N-(3- |
| | fluorobenzyl)-1H-pyrazole-1-carboxamide |
| 1700 | N,5-dicyclopropyl-3-{5-[(4-ethoxybenzoyl)amino]-2-hydroxyphenyl}-1H- |
| | pyrazole-1-carboxamide |
| 1701 | 5-cyclobutyl-3-{5-[(4-ethoxybenzoyl)amino]-2-hydroxyphenyl}-N-isopropyl-1H- |
| | pyrazole-1-carboxamide |
| 1702 | 5-cyclobutyl-3-{5-[(4-ethoxybenzoyl)amino]-2-hydroxyphenyl}-N-ethyl-1H- |
| | pyrazole-1-carboxamide |
| 1703 | N-allyl-5-cyclobutyl-3-{5-[(4-ethoxybenzoyl)amino]-2-hydroxyphenyl}-1H- |
| | pyrazole-1-carboxamide |
| 1704 | 5-cyclobutyl-3-{5-[(4-ethoxybenzoyl)amino]-2-hydroxyphenyl}-N-propyl-1H- |
| | pyrazole-1-carboxamide |
| 1705 | N-benzyl-5-cyclobutyl-3-{5-[(4-ethoxybenzoyl)amino]-2-hydroxyphenyl}-1H- |
| | pyrazole-1-carboxamide |
| 1706 | 5-cyclobutyl-3-{5-[(4-ethoxybenzoyl)amino]-2-hydroxyphenyl}-N-(2- |
| | phenylethyl)-1H-pyrazole-1-carboxamide |
| 1707 | 5-cyclobutyl-3-{5-[(4-ethoxybenzoyl)amino]-2-hydroxyphenyl}-N-(2- |
| | methylbenzyl)-1H-pyrazole-1-carboxamide |
| 1708 | 5-cyclobutyl-3-{5-[(4-ethoxybenzoyl)amino]-2-hydroxyphenyl}-N-(3- |
| | methylbenzyl)-1H-pyrazole-1-carboxamide |
| 1709 | 5-cyclobutyl-3-{5-[(4-ethoxybenzoyl)amino]-2-hydroxyphenyl}-N-(4- |
| | methylbenzyl)-1H-pyrazole-1-carboxamide |
| 1710 | N-(2-chlorobenzyl)-5-cyclobutyl-3-{5-[(4-ethoxybenzoyl)amino]-2- |
| | hydroxyphenyl}-1H-pyrazole-1-carboxamide |
| 1711 | 5-cyclobutyl-3-{5-[(4-ethoxybenzoyl)amino]-2-hydroxyphenyl}-N-(4- |
| | fluorobenzyl)-1H-pyrazole-1-carboxamide |
| 1712 | 5-cyclobutyl-N-(3,4-dichlorobenzyl)-3-{5-[(4-ethoxybenzoyl)amino]-2- |
| | hydroxyphenyl}-1H-pyrazole-1-carboxamide |
| 1713 | 5-cyclobutyl-3-{5-[(4-ethoxybenzoyl)amino]-2-hydroxyphenyl}-N-(4- |
| | methoxybenzyl)-1H-pyrazole-1-carboxamide |
| 1714 | N-(sec-butyl)-5-cyclobutyl-3-{5-[(4-ethoxybenzoyl)amino]-2-hydroxyphenyl}- |
| | 1H-pyrazole-1-carboxamide |
| 1715 | 5-cyclobutyl-3-{5-[(4-ethoxybenzoyl)amino]-2-hydroxyphenyl}-N-(2- |
| | fluorobenzyl)-1H-pyrazole-1-carboxamide |
| 1716 | 5-cyclobutyl-3-{5-[(4-ethoxybenzoyl)amino]-2-hydroxyphenyl}-N-(3- |
| | fluorobenzyl)-1H-pyrazole-1-carboxamide |
| 1717 | 5-cyclobutyl-N-cyclopropyl-3-{5-[(4-ethoxybenzoyl)amino]-2-hydroxyphenyl}- |
| | 1H-pyrazole-1-carboxamide |
| 1718 | 5-cyclopentyl-3-{5-[(4-ethoxybenzoyl)amino]-2-hydroxyphenyl}-N-isopropyl- |
| | 1H-pyrazole-1-carboxamide |
| 1719 | 5-cyclopentyl-3-{5-[(4-ethoxybenzoyl)amino]-2-hydroxyphenyl}-N-ethyl-1H- |
| | pyrazole-1-carboxamide |
| 1720 | N-allyl-5-cyclopentyl-3-{5-[(4-ethoxybenzoyl)amino]-2-hydroxyphenyl}-1H- |
| | pyrazole-1-carboxamide |
| 1721 | 5-cyclopentyl-3-{5-[(4-ethoxybenzoyl)amino]-2-hydroxyphenyl}-N-propyl-1H- |
| | pyrazole-1-carboxamide |
| 1722 | N-benzyl-5-cyclopentyl-3-{5-[(4-ethoxybenzoyl)amino]-2-hydroxyphenyl}-1H- |
| | pyrazole-1-carboxamide |
| 1723 | 5-cyclopentyl-3-{5-[(4-ethoxybenzoyl)amino]-2-hydroxyphenyl}-N-(2- |
| | phenylethyl)-1H-pyrazole-1-carboxamide |
| 1724 | 5-cyclopentyl-3-{5-[(4-ethoxybenzoyl)amino]-2-hydroxyphenyl}-N-(2- |
| | methylbenzyl)-1H-pyrazole-1-carboxamide |
| 1725 | 5-cyclopentyl-3-{5-[(4-ethoxybenzoyl)amino]-2-hydroxyphenyl}-N-(3- |
| | methylbenzyl)-1H-pyrazole-1-carboxamide |
| 1726 | 5-cyclopentyl-3-{5-[(4-ethoxybenzoyl)amino]-2-hydroxyphenyl}-N-(4- |
| | methylbenzyl)-1H-pyrazole-1-carboxamide |
| 1727 | N-(2-chlorobenzyl)-5-cyclopentyl-3-{5-[(4-ethoxybenzoyl)amino]-2- |
| | hydroxyphenyl}-1H-pyrazole-1-carboxamide |
| 1728 | 5-cyclopentyl-3-{5-[(4-ethoxybenzoyl)amino]-2-hydroxyphenyl}-N-(4- |
| | fluorobenzyl)-1H-pyrazole-1-carboxamide |
| 1729 | 5-cyclopentyl-N-(3,4-dichlorobenzyl)-3-{5-[(4-ethoxybenzoyl)amino]-2- |
| | hydroxyphenyl}-1H-pyrazole-1-carboxamide |
| 1730 | 5-cyclopentyl-3-{5-[(4-ethoxybenzoyl)amino]-2-hydroxyphenyl}-N-(4- |
| | methoxybenzyl)-1H-pyrazole-1-carboxamide |
| 1731 | N-(sec-butyl)-5-cyclopentyl-3-{5-[(4-ethoxybenzoyl)amino]-2-hydroxyphenyl}- |
| | 1H-pyrazole-1-carboxamide |
| 1732 | 5-cyclopentyl-3-{5-[(4-ethoxybenzoyl)amino]-2-hydroxyphenyl}-N-(2- |
| | fluorobenzyl)-1H-pyrazole-1-carboxamide |
| 1733 | 5-cyclopentyl-3-{5-[(4-ethoxybenzoyl)amino]-2-hydroxyphenyl}-N-(3- |
| | fluorobenzyl)-1H-pyrazole-1-carboxamide |
| 1734 | 5-cyclopentyl-N-cyclopropyl-3-{5-[(4-ethoxybenzoyl)amino]-2-hydroxyphenyl}- |
| | 1H-pyrazole-1-carboxamide |
| 1735 | 3-{5-[(4-tert-butylbenzoyl)amino]-2-hydroxyphenyl}-5-cyclopropyl-N-isopropyl- |
| | 1H-pyrazole-1-carboxamide |
| 1736 | 3-{5-[(4-tert-butylbenzoyl)amino]-2-hydroxyphenyl}-5-cyclopropyl-N-ethyl-1H- |
| | pyrazole-1-carboxamide |
| 1737 | N-allyl-3-{5-[(4-tert-butylbenzoyl)amino]-2-hydroxyphenyl}-5-cyclopropyl-1H- |
| | pyrazole-1-carboxamide |
| 1738 | 3-{5-[(4-tert-butylbenzoyl)amino]-2-hydroxyphenyl}-5-cyclopropyl-N-propyl- |
| | 1H-pyrazole-1-carboxamide |
| 1739 | N-benzyl-3-{5-[(4-tert-butylbenzoyl)amino]-2-hydroxyphenyl}-5-cyclopropyl- |
| | 1H-pyrazole-1-carboxamide |
| 1740 | 3-{5-[(4-tert-butylbenzoyl)amino]-2-hydroxyphenyl}-5-cyclopropyl-N-(2- |
| | phenylethyl)-1H-pyrazole-1-carboxamide |
| 1741 | 3-{5-[(4-tert-butylbenzoyl)amino]-2-hydroxyphenyl}-5-cyclopropyl-N-(2- |
| | methylbenzyl)-1H-pyrazole-1-carboxamide |
| 1742 | 3-{5-[(4-tert-butylbenzoyl)amino]-2-hydroxyphenyl}-5-cyclopropyl-N-(3- |
| | methylbenzyl)-1H-pyrazole-1-carboxamide |
| 1743 | 3-{5-[(4-tert-butylbenzoyl)amino]-2-hydroxyphenyl}-5-cyclopropyl-N-(4- |
| | methylbenzyl)-1H-pyrazole-1-carboxamide |
| 1744 | 3-{5-[(4-tert-butylbenzoyl)amino]-2-hydroxyphenyl}-N-(2-chlorobenzyl)-5- |
| | cyclopropyl-1H-pyrazole-1-carboxamide |
| 1745 | 3-{5-[(4-tert-butylbenzoyl)amino]-2-hydroxyphenyl}-5-cyclopropyl-N-(4- |
| | fluorobenzyl)-1H-pyrazole-1-carboxamide |
| 1746 | 3-{5-[(4-tert-butylbenzoyl)amino]-2-hydroxyphenyl}-5-cyclopropyl-N-(3,4- |
| | dichlorobenzyl)-1H-pyrazole-1-carboxamide |
| 1747 | 3-{5-[(4-tert-butylbenzoyl)amino]-2-hydroxyphenyl}-5-cyclopropyl-N-(4- |
| | methoxybenzyl)-1H-pyrazole-1-carboxamide |
| 1748 | N-(sec-butyl)-3-{5-[(4-tert-butylbenzoyl)amino]-2-hydroxyphenyl}-5- |
| | cyclopropyl-1H-pyrazole-1-carboxamide |
| 1749 | 3-{5-[(4-tert-butylbenzoyl)amino]-2-hydroxyphenyl}-5-cyclopropyl-N-(2- |
| | fluorobenzyl)-1H-pyrazole-1-carboxamide |
| 1750 | 3-{5-[(4-tert-butylbenzoyl)amino]-2-hydroxyphenyl}-5-cyclopropyl-N-(3- |
| | fluorobenzyl)-1H-pyrazole-1-carboxamide |
| 1751 | 3-{5-[(4-tert-butylbenzoyl)amino]-2-hydroxyphenyl}-N,5-dicyclopropyl-1H- |
| | pyrazole-1-carboxamide |
| 1752 | 3-{5-[(4-tert-butylbenzoyl)amino]-2-hydroxyphenyl}-5-cyclobutyl-N-isopropyl- |
| | 1H-pyrazole-1-carboxamide |
| 1753 | 3-{5-[(4-tert-butylbenzoyl)amino]-2-hydroxyphenyl}-5-cyclobutyl-N-ethyl-1H- |
| | pyrazole-1-carboxamide |
| 1754 | N-allyl-3-{5-[(4-tert-butylbenzoyl)amino]-2-hydroxyphenyl}-5-cyclobutyl-1H- |
| | pyrazole-1-carboxamide |
| 1755 | 3-{5-[(4-tert-butylbenzoyl)amino]-2-hydroxyphenyl}-5-cyclobutyl-N-propyl-1H- |
| | pyrazole-1-carboxamide |
| 1756 | N-benzyl-3-{5-[(4-tert-butylbenzoyl)amino]-2-hydroxyphenyl}-5-cyclobutyl-1H- |
| | pyrazole-1-carboxamide |
| 1757 | 3-{5-[(4-tert-butylbenzoyl)amino]-2-hydroxyphenyl}-5-cyclobutyl-N-(2- |
| | phenylethyl)-1H-pyrazole-1-carboxamide |
| 1758 | 3-{5-[(4-tert-butylbenzoyl)amino]-2-hydroxyphenyl}-5-cyclobutyl-N-(2- |
| | methylbenzyl)-1H-pyrazole-1-carboxamide |
| 1759 | 3-{5-[(4-tert-butylbenzoyl)amino]-2-hydroxyphenyl}-5-cyclobutyl-N-(3- |
| | methylbenzyl)-1H-pyrazole-1-carboxamide |
| 1760 | 3-{5-[(4-tert-butylbenzoyl)amino]-2-hydroxyphenyl}-5-cyclobutyl-N-(4- |
| | methylbenzyl)-1H-pyrazole-1-carboxamide |
| 1761 | 3-{5-[(4-tert-butylbenzoyl)amino]-2-hydroxyphenyl}-N-(2-chlorobenzyl)-5- |
| | cyclobutyl-1H-pyrazole-1-carboxamide |
| 1762 | 3-{5-[(4-tert-butylbenzoyl)amino]-2-hydroxyphenyl}-5-cyclobutyl-N-(4- |
| | fluorobenzyl)-1H-pyrazole-1-carboxamide |
| 1763 | 3-{5-[(4-tert-butylbenzoyl)amino]-2-hydroxyphenyl}-5-cyclobutyl-N-(3,4- |
| | dichlorobenzyl)-1H-pyrazole-1-carboxamide |
| 1764 | 3-{5-[(4-tert-butylbenzoyl)amino]-2-hydroxyphenyl}-5-cyclobutyl-N-(4- |
| | methoxybenzyl)-1H-pyrazole-1-carboxamide |
| 1765 | N-(sec-butyl)-3-{5-[(4-tert-butylbenzoyl)amino]-2-hydroxyphenyl}-5-cyclobutyl- |
| | 1H-pyrazole-1-carboxamide |
| 1766 | 3-{5-[(4-tert-butylbenzoyl)amino]-2-hydroxyphenyl}-5-cyclobutyl-N-(2- |
| | fluorobenzyl)-1H-pyrazole-1-carboxamide |
| 1767 | 3-{5-[(4-tert-butylbenzoyl)amino]-2-hydroxyphenyl}-5-cyclobutyl-N- |
| | cyclopropyl-1H-pyrazole-1-carboxamide |
| 1768 | 3-{5-[(4-tert-butylbenzoyl)amino]-2-hydroxyphenyl}-5-cyclobutyl-N- |
| | cyclopropyl-1H-pyrazole-1-carboxamide |
| 1769 | 3-{5-[(4-tert-butylbenzoyl)amino]-2-hydroxyphenyl}-5-cyclopentyl-N-isopropyl- |
| | 1H-pyrazole-1-carboxamide |
| 1770 | 3-{5-[(4-tert-butylbenzoyl)amino]-2-hydroxyphenyl}-5-cyclopentyl-N-ethyl-1H- |
| | pyrazole-1-carboxamide |
| 1771 | N-allyl-3-{5-[(4-tert-butylbenzoyl)amino]-2-hydroxyphenyl}-5-cyclopentyl-1H- |
| | pyrazole-1-carboxamide |
| 1772 | 3-{5-[(4-tert-butylbenzoyl)amino]-2-hydroxyphenyl}-5-cyclopentyl-N-propyl- |
| | 1H-pyrazole-1-carboxamide |
| 1773 | N-benzyl-3-{5-[(4-tert-butylbenzoyl)amino]-2-hydroxyphenyl}-5-cyclopentyl- |
| | 1H-pyrazole-1-carboxamide |
| 1774 | 3-{5-[(4-tert-butylbenzoyl)amino]-2-hydroxyphenyl}-5-cyclopentyl-N-(2- |
| | phenylethyl)-1H-pyrazole-1-carboxamide |
| 1775 | 3-{5-[(4-tert-butylbenzoyl)amino]-2-hydroxyphenyl}-5-cyclopentyl-N-(2- |
| | methylbenzyl)-1H-pyrazole-1-carboxamide |
| 1776 | 3-{5-[(4-tert-butylbenzoyl)amino]-2-hydroxyphenyl}-5-cyclopentyl-N-(3- |
| | methylbenzyl)-1H-pyrazole-1-carboxamide |
| 1777 | 3-{5-[(4-tert-butylbenzoyl)amino]-2-hydroxyphenyl}-5-cyclopentyl-N-(4- |
| | methylbenzyl)-1H-pyrazole-1-carboxamide |
| 1778 | 3-{5-[(4-tert-butylbenzoyl)amino]-2-hydroxyphenyl}-N-(2-chlorobenzyl)-5- |
| | cyclopentyl-1H-pyrazole-1-carboxamide |
| 1779 | 3-{5-[(4-tert-butylbenzoyl)amino]-2-hydroxyphenyl}-5-cyclopentyl-N-(4- |
| | fluorobenzyl)-1H-pyrazole-1-carboxamide |
| 1780 | 3-{5-[(4-tert-butylbenzoyl)amino]-2-hydroxyphenyl}-5-cyclopentyl-N-(3,4- |
| | dichlorobenzyl)-1H-pyrazole-1-carboxamide |
| 1781 | 3-{5-[(4-tert-butylbenzoyl)amino]-2-hydroxyphenyl}-5-cyclopentyl-N-(4- |
| | methoxybenzyl)-1H-pyrazole-1-carboxamide |
| 1782 | N-(sec-butyl)-3-{5-[(4-tert-butylbenzoyl)amino]-2-hydroxyphenyl}-5- |
| | cyclopentyl-1H-pyrazole-1-carboxamide |
| 1783 | 3-{5-[(4-tert-butylbenzoyl)amino]-2-hydroxyphenyl}-5-cyclopentyl-N-(2- |
| | fluorobenzyl)-1H-pyrazole-1-carboxamide |
| 1784 | 3-{5-[(4-tert-butylbenzoyl)amino]-2-hydroxyphenyl}-5-cyclopentyl-N-(3- |
| | fluorobenzyl)-1H-pyrazole-1-carboxamide |
| 1785 | 3-{5-[(4-tert-butylbenzoyl)amino]-2-hydroxyphenyl}-5-cyclopentyl-N- |
| | cyclopropyl-1H-pyrazole-1-carboxamide |
| 1786 | 5-cyclopropyl-3-{2-hydroxy-5-[(4-methylbenzoyl)amino]phenyl}-N-isopropyl- |
| | 1H-pyrazole-1-carboxamide |
| 1787 | 5-cyclopropyl-N-ethyl-3-{2-hydroxy-5-[(4-methylbenzoyl)amino]phenyl}-1H- |
| | pyrazole-1-carboxamide |
| 1788 | N-allyl-5-cyclopropyl-3-{2-hydroxy-5-[(4-methylbenzoyl)amino]phenyl}-1H- |
| | pyrazole-1-carboxamide |
| 1789 | 5-cyclopropyl-3-{2-hydroxy-5-[(4-methylbenzoyl)amino]phenyl}-N-propyl-1H- |
| | pyrazole-1-carboxamide |
| 1790 | N-benzyl-5-cyclopropyl-3-{2-hydroxy-5-[(4-methylbenzoyl)amino]phenyl}-1H- |
| | pyrazole-1-carboxamide |
| 1791 | 5-cyclopropyl-3-{2-hydroxy-5-[(4-methylbenzoyl)amino]phenyl}-N-(2- |
| | phenylethyl)-1H-pyrazole-1-carboxamide |
| 1792 | 5-cyclopropyl-3-{2-hydroxy-5-[(4-methylbenzoyl)amino]phenyl}-N-(2- |
| | methylbenzyl)-1H-pyrazole-1-carboxamide |
| 1793 | 5-cyclopropyl-3-{2-hydroxy-5-[(4-methylbenzoyl)amino]phenyl}-N-(3- |
| | methylbenzyl)-1H-pyrazole-1-carboxamide |
| 1794 | 5-cyclopropyl-3-{2-hydroxy-5-[(4-methylbenzoyl)amino]phenyl}-N-(4- |
| | methylbenzyl)-1H-pyrazole-1-carboxamide |
| 1795 | N-(2-chlorobenzyl)-5-cyclopropyl-3-{2-hydroxy-5-[(4- |
| | methylbenzoyl)amino]phenyl}-1H-pyrazole-1-carboxamide |
| 1796 | 5-cyclopropyl-N-(4-fluorobenzyl)-3-{2-hydroxy-5-[(4- |
| | methylbenzoyl)amino]phenyl}-1H-pyrazole-1-carboxamide |
| 1797 | 5-cyclopropyl-N-(3,4-dichlorobenzyl)-3-{2-hydroxy-5-[(4- |
| | methylbenzoyl)amino]phenyl}-1H-pyrazole-1-carboxamide |
| 1798 | 5-cyclopropyl-3-{2-hydroxy-5-[(4-methylbenzoyl)amino]phenyl}-N-(4- |
| | methoxybenzyl)-1H-pyrazole-1-carboxamide |
| 1799 | N-(sec-butyl)-5-cyclopropyl-3-{2-hydroxy-5-[(4-methylbenzoyl)amino]phenyl}- |
| | 1H-pyrazole-1-carboxamide |
| 1800 | 5-cyclopropyl-N-(2-fluorobenzyl)-3-{2-hydroxy-5-[(4- |
| | methylbenzoyl)amino]phenyl}-1H-pyrazole-1-carboxamide |
| 1801 | 5-cyclopropyl-N-(3-fluorobenzyl)-3-{2-hydroxy-5-[(4- |
| | methylbenzoyl)amino]phenyl}-1H-pyrazole-1-carboxamide |
| 1802 | N,5-dicyclopropyl-3-{2-hydroxy-5-[(4-methylbenzoyl)amino]phenyl}-1H- |
| | pyrazole-1-carboxamide |
| 1803 | 5-cyclobutyl-3-{2-hydroxy-5-[(4-methylbenzoyl)amino]phenyl}-N-isopropyl-1H- |
| | pyrazole-1-carboxamide |
| 1804 | 5-cyclobutyl-N-ethyl-3-{2-hydroxy-5-[(4-methylbenzoyl)amino]phenyl}-1H- |
| | pyrazole-1-carboxamide |
| 1805 | N-allyl-5-cyclobutyl-3-{2-hydroxy-5-[(4-methylbenzoyl)amino]phenyl}-1H- |
| | pyrazole-1-carboxamide |
| 1806 | 5-cyclobutyl-3-{2-hydroxy-5-[(4-methylbenzoyl)amino]phenyl}-N-propyl-1H- |
| | pyrazole-1-carboxamide |
| 1807 | N-benzyl-5-cyclobutyl-3-{2-hydroxy-5-[(4-methylbenzoyl)amino]phenyl}-1H- |
| | pyrazole-1-carboxamide |
| 1808 | 5-cyclobutyl-3-{2-hydroxy-5-[(4-methylbenzoyl)amino]phenyl}-N-(2- |
| | phenylethyl)-1H-pyrazole-1-carboxamide |
| 1809 | 5-cyclobutyl-3-{2-hydroxy-5-[(4-methylbenzoyl)amino]phenyl}-N-(2- |
| | methylbenzyl)-1H-pyrazole-1-carboxamide |
| 1810 | 5-cyclobutyl-3-{2-hydroxy-5-[(4-methylbenzoyl)amino]phenyl}-N-(3- |
| | methylbenzyl)-1H-pyrazole-1-carboxamide |
| 1811 | 5-cyclobutyl-3-{2-hydroxy-5-[(4-methylbenzoyl)amino]phenyl}-N-(4- |
| | methylbenzyl)-1H-pyrazole-1-carboxamide |
| 1812 | N-(2-chlorobenzyl)-5-cyclobutyl-3-{2-hydroxy-5-[(4- |
| | methylbenzoyl)amino]phenyl}-1H-pyrazole-1-carboxamide |
| 1813 | 5-cyclobutyl-N-(4-fluorobenzyl)-3-{2-hydroxy-5-[(4- |
| | methylbenzoyl)amino]phenyl}-1H-pyrazole-1-carboxamide |
| 1814 | 5-cyclobutyl-N-(3,4-dichlorobenzyl)-3-{2-hydroxy-5-[(4- |
| | methylbenzoyl)amino]phenyl}-1H-pyrazole-1-carboxamide |
| 1815 | 5-cyclobutyl-3-{2-hydroxy-5-[(4-methylbenzoyl)amino]phenyl}-N-(4- |
| | methoxybenzyl)-1H-pyrazole-1-carboxamide |
| 1816 | N-(sec-butyl)-5-cyclobutyl-3-{2-hydroxy-5-[(4-methylbenzoyl)amino]phenyl}- |
| | 1H-pyrazole-1-carboxamide |
| 1817 | 5-cyclobutyl-N-(2-fluorobenzyl)-3-{2-hydroxy-5-[(4- |
| | methylbenzoyl)amino]phenyl}-1H-pyrazole-1-carboxamide |
| 1818 | 5-cyclobutyl-N-(3-fluorobenzyl)-3-{2-hydroxy-5-[(4- |
| | methylbenzoyl)amino]phenyl}-1H-pyrazole-1-carboxamide |
| 1819 | 5-cyclobutyl-N-cyclopropyl-3-{2-hydroxy-5-[(4-methylbenzoyl)amino]phenyl}- |
| | 1H-pyrazole-1-carboxamide |
| 1820 | 5-cyclopentyl-3-{2-hydroxy-5-[(4-methylbenzoyl)amino]phenyl}-N-isopropyl- |
| | 1H-pyrazole-1-carboxamide |
| 1821 | 5-cyclopentyl-N-ethyl-3-{2-hydroxy-5-[(4-methylbenzoyl)amino]phenyl}-1H- |
| | pyrazole-1-carboxamide |
| 1822 | N-allyl-5-cyclopentyl-3-{2-hydroxy-5-[(4-methylbenzoyl)amino]phenyl}-1H- |
| | pyrazole-1-carboxamide |
| 1823 | 5-cyclopentyl-3-{2-hydroxy-5-[(4-methylbenzoyl)amino]phenyl}-N-propyl-1H- |
| | pyrazole-1-carboxamide |
| 1824 | N-benzyl-5-cyclopentyl-3-{2-hydroxy-5-[(4-methylbenzoyl)amino]phenyl}-1H- |
| | pyrazole-1-carboxamide |
| 1825 | 5-cyclopentyl-3-{2-hydroxy-5-[(4-methylbenzoyl)amino]phenyl}-N-(2- |
| | phenylethyl)-1H-pyrazole-1-carboxamide |
| 1826 | 5-cyclopentyl-3-{2-hydroxy-5-[(4-methylbenzoyl)amino]phenyl}-N-(2- |
| | methylbenzyl)-1H-pyrazole-1-carboxamide |
| 1827 | 5-cyclopentyl-3-{2-hydroxy-5-[(4-methylbenzoyl)amino]phenyl}-N-(3- |
| | methylbenzyl)-1H-pyrazole-1-carboxamide |
| 1828 | 5-cyclopentyl-3-{2-hydroxy-5-[(4-methylbenzoyl)amino]phenyl}-N-(4- |
| | methylbenzyl)-1H-pyrazole-1-carboxamide |
| 1829 | N-(2-chlorobenzyl)-5-cyclopentyl-3-{2-hydroxy-5-[(4- |
| | methylbenzoyl)amino]phenyl}-1H-pyrazole-1-carboxamide |
| 1830 | 5-cyclopentyl-N-(4-fluorobenzyl)-3-{2-hydroxy-5-[(4- |
| | methylbenzoyl)amino]phenyl}-1H-pyrazole-1-carboxamide |
| 1831 | 5-cyclopentyl-N-(3,4-dichlorobenzyl)-3-{2-hydroxy-5-[(4- |
| | methylbenzoyl)amino]phenyl}-1H-pyrazole-1-carboxamide |
| 1832 | 5-cyclopentyl-3-{2-hydroxy-5-[(4-methylbenzoyl)amino]phenyl}-N-(4- |
| | methoxybenzyl)-1H-pyrazole-1-carboxamide |
| 1833 | N-(sec-butyl)-5-cyclopentyl-3-{2-hydroxy-5-[(4-methylbenzoyl)amino]phenyl}- |
| | 1H-pyrazole-1-carboxamide |
| 1834 | 5-cyclopentyl-N-(2-fluorobenzyl)-3-{2-hydroxy-5-[(4- |
| | methylbenzoyl)amino]phenyl}-1H-pyrazole-1-carboxamide |
| 1835 | 5-cyclopentyl-N-(3-fluorobenzyl)-3-{2-hydroxy-5-[(4- |
| | methylbenzoyl)amino]phenyl}-1H-pyrazole-1-carboxamide |
| 1836 | 5-cyclopentyl-N-cyclopropyl-3-{2-hydroxy-5-[(4-methylbenzoyl)amino]phenyl}- |
| | 1H-pyrazole-1-carboxamide |
| 1837 | 5-cyclopropyl-3-{5-[(4-ethylbenzoyl)amino]-2-hydroxyphenyl}-N-isopropyl-1H- |
| | pyrazole-1-carboxamide |
| 1838 | 5-cyclopropyl-N-ethyl-3-{5-[(4-ethylbenzoyl)amino]-2-hydroxyphenyl}-1H- |
| | pyrazole-1-carboxamide |
| 1839 | N-allyl-5-cyclopropyl-3-{5-[(4-ethylbenzoyl)amino]-2-hydroxyphenyl}-1H- |
| | pyrazole-1-carboxamide |
| 1840 | 5-cyclopropyl-3-{5-[(4-ethylbenzoyl)amino]-2-hydroxyphenyl}-N-propyl-1H- |
| | pyrazole-1-carboxamide |
| 1841 | N-benzyl-5-cyclopropyl-3-{5-[(4-ethylbenzoyl)amino]-2-hydroxyphenyl}-1H- |
| | pyrazole-1-carboxamide |
| 1842 | 5-cyclopropyl-3-{5-[(4-ethylbenzoyl)amino]-2-hydroxyphenyl}-N-(2- |
| | phenylethyl)-1H-pyrazole-1-carboxamide |
| 1843 | 5-cyclopropyl-3-{5-[(4-ethylbenzoyl)amino]-2-hydroxyphenyl}-N-(2- |
| | methylbenzyl)-1H-pyrazole-1-carboxamide |
| 1844 | 5-cyclopropyl-3-{5-[(4-ethylbenzoyl)amino]-2-hydroxyphenyl}-N-(3- |
| | methylbenzyl)-1H-pyrazole-1-carboxamide |
| 1845 | 5-cyclopropyl-3-{5-[(4-ethylbenzoyl)amino]-2-hydroxyphenyl}-N-(4- |
| | methylbenzyl)-1H-pyrazole-1-carboxamide |
| 1846 | N-(2-chlorobenzyl)-5-cyclopropyl-3-{5-[(4-ethylbenzoyl)amino]-2- |
| | hydroxyphenyl}-1H-pyrazole-1-carboxamide |
| 1847 | 5-cyclopropyl-3-{5-[(4-ethylbenzoyl)amino]-2-hydroxyphenyl}-N-(4- |
| | fluorobenzyl)-1H-pyrazole-1-carboxamide |
| 1848 | 5-cyclopropyl-N-(3,4-dichlorobenzyl)-3-{5-[(4-ethylbenzoyl)amino]-2- |
| | hydroxyphenyl}-1H-pyrazole-1-carboxamide |
| 1849 | 5-cyclopropyl-3-{5-[(4-ethylbenzoyl)amino]-2-hydroxyphenyl}-N-(4- |
| | methoxybenzyl)-1H-pyrazole-1-carboxamide |
| 1850 | N-(sec-butyl)-5-cyclopropyl-3-{5-[(4-ethylbenzoyl)amino]-2-hydroxyphenyl}- |
| | 1H-pyrazole-1-carboxamide |
| 1851 | 5-cyclopropyl-3-{5-[(4-ethylbenzoyl)amino]-2-hydroxyphenyl}-N-(2- |
| | fluorobenzyl)-1H-pyrazole-1-carboxamide |
| 1852 | 5-cyclopropyl-3-{5-[(4-ethylbenzoyl)amino]-2-hydroxyphenyl}-N-(3- |
| | fluorobenzyl)-1H-pyrazole-1-carboxamide |
| 1853 | N,5-dicyclopropyl-3-{5-[(4-ethylbenzoyl)amino]-2-hydroxyphenyl}-1H-pyrazole- |
| | 1-carboxamide |
| 1854 | 5-cyclobutyl-3-{5-[(4-ethylbenzoyl)amino]-2-hydroxyphenyl}-N-isopropyl-1H- |
| | pyrazole-1-carboxamide |
| 1855 | 5-cyclobutyl-N-ethyl-3-{5-[(4-ethylbenzoyl)amino]-2-hydroxyphenyl}-1H- |
| | pyrazole-1-carboxamide |
| 1856 | N-allyl-5-cyclobutyl-3-{5-[(4-ethylbenzoyl)amino]-2-hydroxyphenyl}-1H- |
| | pyrazole-1-carboxamide |
| 1857 | 5-cyclobutyl-3-{5-[(4-ethylbenzoyl)amino]-2-hydroxyphenyl}-N-propyl-1H- |
| | pyrazole-1-carboxamide |
| 1858 | N-benzyl-5-cyclobutyl-3-{5-[(4-ethylbenzoyl)amino]-2-hydroxyphenyl}-1H- |
| | pyrazole-1-carboxamide |
| 1859 | 5-cyclobutyl-3-{5-[(4-ethylbenzoyl)amino]-2-hydroxyphenyl}-N-(2-phenylethyl)- |
| | 1H-pyrazole-1-carboxamide |
| 1860 | 5-cyclobutyl-3-{5-[(4-ethylbenzoyl)amino]-2-hydroxyphenyl}-N-(2- |
| | methylbenzyl)-1H-pyrazole-1-carboxamide |
| 1861 | 5-cyclobutyl-3-{5-[(4-ethylbenzoyl)amino]-2-hydroxyphenyl}-N-(3- |
| | methylbenzyl)-1H-pyrazole-1-carboxamide |
| 1862 | 5-cyclobutyl-3-{5-[(4-ethylbenzoyl)amino]-2-hydroxyphenyl}-N-(4- |
| | methylbenzyl)-1H-pyrazole-1-carboxamide |
| 1863 | N-(2-chlorobenzyl)-5-cyclobutyl-3-{5-[(4-ethylbenzoyl)amino]-2- |
| | hydroxyphenyl}-1H-pyrazole-1-carboxamide |
| 1864 | 5-cyclobutyl-3-{5-[(4-ethylbenzoyl)amino]-2-hydroxyphenyl}-N-(4- |
| | fluorobenzyl)-1H-pyrazole-1-carboxamide |
| 1865 | 5-cyclobutyl-N-(3,4-dichlorobenzyl)-3-{5-[(4-ethylbenzoyl)amino]-2- |
| | hydroxyphenyl}-1H-pyrazole-1-carboxamide |
| 1866 | 5-cyclobutyl-3-{5-[(4-ethylbenzoyl)amino]-2-hydroxyphenyl}-N-(4- |
| | methoxybenzyl)-1H-pyrazole-1-carboxamide |
| 1867 | N-(sec-butyl)-5-cyclobutyl-3-{5-[(4-ethylbenzoyl)amino]-2-hydroxyphenyl}-1H- |
| | pyrazole-1-carboxamide |
| 1868 | 5-cyclobutyl-3-{5-[(4-ethylbenzoyl)amino]-2-hydroxyphenyl}-N-(2- |
| | fluorobenzyl)-1H-pyrazole-1-carboxamide |
| 1869 | 5-cyclobutyl-3-{5-[(4-ethylbenzoyl)amino]-2-hydroxyphenyl}-N-(3- |
| | fluorobenzyl)-1H-pyrazole-1-carboxamide |
| 1870 | 5-cyclobutyl-N-cyclopropyl-3-{5-[(4-ethylbenzoyl)amino]-2-hydroxyphenyl}- |
| | 1H-pyrazole-1-carboxamide |
| 1871 | 5-cyclopentyl-3-{5-[(4-ethylbenzoyl)amino]-2-hydroxyphenyl}-N-isopropyl-1H- |
| | pyrazole-1-carboxamide |
| 1872 | 5-cyclopentyl-N-ethyl-3-{5-[(4-ethylbenzoyl)amino]-2-hydroxyphenyl}-1H- |
| | pyrazole-1-carboxamide |
| 1873 | N-allyl-5-cyclopentyl-3-{5-[(4-ethylbenzoyl)amino]-2-hydroxyphenyl}-1H- |
| | pyrazole-1-carboxamide |
| 1874 | 5-cyclopentyl-3-{5-[(4-ethylbenzoyl)amino]-2-hydroxyphenyl}-N-propyl-1H- |
| | pyrazole-1-carboxamide |
| 1875 | N-benzyl-5-cyclopentyl-3-{5-[(4-ethylbenzoyl)amino]-2-hydroxyphenyl}-1H- |
| | pyrazole-1-carboxamide |
| 1876 | 5-cyclopentyl-3-{5-[(4-ethylbenzoyl)amino]-2-hydroxyphenyl}-N-(2- |
| | phenylethyl)-1H-pyrazole-1-carboxamide |
| 1877 | 5-cyclopentyl-3-{5-[(4-ethylbenzoyl)amino]-2-hydroxyphenyl}-N-(2- |
| | methylbenzyl)-1H-pyrazole-1-carboxamide |
| 1878 | 5-cyclopentyl-3-{5-[(4-ethylbenzoyl)amino]-2-hydroxyphenyl}-N-(3- |
| | methylbenzyl)-1H-pyrazole-1-carboxamide |
| 1879 | 5-cyclopentyl-3-{5-[(4-ethylbenzoyl)amino]-2-hydroxyphenyl}-N-(4- |
| | methylbenzyl)-1H-pyrazole-1-carboxamide |
| 1880 | N-(2-chlorobenzyl)-5-cyclopentyl-3-{5-[(4-ethylbenzoyl)amino]-2- |
| | hydroxyphenyl}-1H-pyrazole-1-carboxamide |
| 1881 | 5-cyclopentyl-3-{5-[(4-ethylbenzoyl)amino]-2-hydroxyphenyl}-N-(4- |
| | fluorobenzyl)-1H-pyrazole-1-carboxamide |
| 1882 | 5-cyclopentyl-N-(3,4-dichlorobenzyl)-3-{5-[(4-ethylbenzoyl)amino]-2- |
| | hydroxyphenyl}-1H-pyrazole-1-carboxamide |
| 1883 | 5-cyclopentyl-3-{5-[(4-ethylbenzoyl)amino]-2-hydroxyphenyl}-N-(4- |
| | methoxybenzyl)-1H-pyrazole-1-carboxamide |
| 1884 | N-(sec-butyl)-5-cyclopentyl-3-{5-[(4-ethylbenzoyl)amino]-2-hydroxyphenyl}- |
| | 1H-pyrazole-1-carboxamide |
| 1885 | 5-cyclopentyl-3-{5-[(4-ethylbenzoyl)amino]-2-hydroxyphenyl}-N-(2- |
| | fluorobenzyl)-1H-pyrazole-1-carboxamide |
| 1886 | 5-cyclopentyl-3-{5-[(4-ethylbenzoyl)amino]-2-hydroxyphenyl}-N-(3- |
| | fluorobenzyl)-1H-pyrazole-1-carboxamide |
| 1887 | 5-cyclopentyl-N-cyclopropyl-3-{5-[(4-ethylbenzoyl)amino]-2-hydroxyphenyl}- |
| | 1H-pyrazole-1-carboxamide |
| 1888 | 5-cyclopropyl-3-[2-hydroxy-5-(isobutyrylamino)phenyl]-N-isopropyl-1H- |
| | pyrazole-1-carboxamide |
| 1889 | 5-cyclopropyl-N-ethyl-3-[2-hydroxy-5-(isobutyrylamino)phenyl]-1H-pyrazole-1- |
| | carboxamide |
| 1890 | N-allyl-5-cyclopropyl-3-[2-hydroxy-5-(isobutyrylamino)phenyl]-1H-pyrazole-1- |
| | carboxamide |
| 1891 | 5-cyclopropyl-3-[2-hydroxy-5-(isobutyrylamino)phenyl]-N-propyl-1H-pyrazole- |
| | 1-carboxamide |
| 1892 | N-benzyl-5-cyclopropyl-3-[2-hydroxy-5-(isobutyrylamino)phenyl]-1H-pyrazole- |
| | 1-carboxamide |
| 1893 | 5-cyclopropyl-3-[2-hydroxy-5-(isobutyrylamino)phenyl]-N-(2-phenylethyl)-1H- |
| | pyrazole-1-carboxamide |
| 1894 | 5-cyclopropyl-3-[2-hydroxy-5-(isobutyrylamino)phenyl]-N-(2-methylbenzyl)-1H- |
| | pyrazole-1-carboxamide |
| 1895 | 5-cyclopropyl-3-[2-hydroxy-5-(isobutyrylamino)phenyl]-N-(3-methylbenzyl)-1H- |
| | pyrazole-1-carboxamide |
| 1896 | 5-cyclopropyl-3-[2-hydroxy-5-(isobutyrylamino)phenyl]-N-(4-methylbenzyl)-1H- |
| | pyrazole-1-carboxamide |
| 1897 | N-(2-chlorobenzyl)-5-cyclopropyl-3-[2-hydroxy-5-(isobutyrylamino)phenyl]-1H- |
| | pyrazole-1-carboxamide |
| 1898 | 5-cyclopropyl-N-(4-fluorobenzyl)-3-[2-hydroxy-5-(isobutyrylamino)phenyl]-1H- |
| | pyrazole-1-carboxamide |
| 1899 | 5-cyclopropyl-N-(3,4-dichlorobenzyl)-3-[2-hydroxy-5-(isobutyrylamino)phenyl]- |
| | 1H-pyrazole-1-carboxamide |
| 1900 | 5-cyclopropyl-3-[2-hydroxy-5-(isobutyrylamino)phenyl]-N-(4-methoxybenzyl)- |
| | 1H-pyrazole-1-carboxamide |
| 1901 | N-(sec-butyl)-5-cyclopropyl-3-[2-hydroxy-5-(isobutyrylamino)phenyl]-1H- |
| | pyrazole-1-carboxamide |
| 1902 | 5-cyclopropyl-N-(2-fluorobenzyl)-3-[2-hydroxy-5-(isobutyrylamino)phenyl]-1H- |
| | pyrazole-1-carboxamide |
| 1903 | 5-cyclopropyl-N-(3-fluorobenzyl)-3-[2-hydroxy-5-(isobutyrylamino)phenyl]-1H- |
| | pyrazole-1-carboxamide |
| 1904 | N,5-dicyclopropyl-3-[2-hydroxy-5-(isobutyrylamino)phenyl]-1H-pyrazole-1- |
| | carboxamide |
| 1905 | 5-cyclobutyl-3-[2-hydroxy-5-(isobutyrylamino)phenyl]-N-isopropyl-1H-pyrazole- |
| | 1-carboxamide |
| 1906 | 5-cyclobutyl-N-ethyl-3-[2-hydroxy-5-(isobutyrylamino)phenyl]-1H-pyrazole-1- |
| | carboxamide |
| 1907 | N-allyl-5-cyclobutyl-3-[2-hydroxy-5-(isobutyrylamino)phenyl]-1H-pyrazole-1- |
| | carboxamide |
| 1908 | 5-cyclobutyl-3-[2-hydroxy-5-(isobutyrylamino)phenyl]-N-propyl-1H-pyrazole-1- |
| | carboxamide |
| 1909 | N-benzyl-5-cyclobutyl-3-[2-hydroxy-5-(isobutyrylamino)phenyl]-1H-pyrazole-1- |
| | carboxamide |
| 1910 | 5-cyclobutyl-3-[2-hydroxy-5-(isobutyrylamino)phenyl]-N-(2-phenylethyl)-1H- |
| | pyrazole-1-carboxamide |
| 1911 | 5-cyclobutyl-3-[2-hydroxy-5-(isobutyrylamino)phenyl]-N-(2-methylbenzyl)-1H- |
| | pyrazole-1-carboxamide |
| 1912 | 5-cyclobutyl-3-[2-hydroxy-5-(isobutyrylamino)phenyl]-N-(3-methylbenzyl)-1H- |
| | pyrazole-1-carboxamide |
| 1913 | 5-cyclobutyl-3-[2-hydroxy-5-(isobutyrylamino)phenyl]-N-(4-methylbenzyl)-1H- |
| | pyrazole-1-carboxamide |
| 1914 | N-(2-chlorobenzyl)-5-cyclobutyl-3-[2-hydroxy-5-(isobutyrylamino)phenyl]-1H- |
| | pyrazole-1-carboxamide |
| 1915 | 5-cyclobutyl-N-(4-fluorobenzyl)-3-[2-hydroxy-5-(isobutyrylamino)phenyl]-1H- |
| | pyrazole-1-carboxamide |
| 1916 | 5-cyclobutyl-N-(3,4-dichlorobenzyl)-3-[2-hydroxy-5-(isobutyrylamino)phenyl]- |
| | 1H-pyrazole-1-carboxamide |
| 1917 | 5-cyclobutyl-3-[2-hydroxy-5-(isobutyrylamino)phenyl]-N(-4-methoxybenzyl)- |
| | 1H-pyrazole-1-carboxamide |
| 1918 | N-(sec-butyl)-5-cyclobutyl-3-[2-hydroxy-5-(isobutyrylamino)phenyl]-1H- |
| | pyrazole-1-carboxamide |
| 1919 | 5-cyclobutyl-N-(2-fluorobenzyl)-3-[2-hydroxy-5-(isobutyrylamino)phenyl]-1H- |
| | pyrazole-1-carboxamide |
| 1920 | 5-cyclobutyl-N-(3-fluorobenzyl)-3-[2-hydroxy-5-(isobutyrylamino)phenyl]-1H- |
| | pyrazole-1-carboxamide |
| 1921 | 5-cyclobutyl-N-cyclopropyl-3-[2-hydroxy-5-(isobutyrylamino)phenyl]-1H- |
| | pyrazole-1-carboxamide |
| 1922 | 5-cyclopentyl-3-[2-hydroxy-5-(isobutyrylamino)phenyl]-N-isopropyl-1H- |
| | pyrazole-1-carboxamide |
| 1923 | 5-cyclopentyl-N-ethyl-3-[2-hydroxy-5-(isobutyrylamino)phenyl]-1H-pyrazole-1- |
| | carboxamide |
| 1924 | N-allyl-5-cyclopentyl-3-[2-hydroxy-5-(isobutyrylamino)phenyl]-1H-pyrazole-1- |
| | carboxamide |
| 1925 | 5-cyclopentyl-3-[2-hydroxy-5-(isobutyrylamino)phenyl]-N-propyl-1H-pyrazole- |
| | 1-carboxamide |
| 1926 | N-benzyl-5-cyclopentyl-3-[2-hydroxy-5-(isobutyrylamino)phenyl]-1H-pyrazole- |
| | 1-carboxamide |
| 1927 | 5-cyclopentyl-3-[2-hydroxy-5-(isobutyrylamino)phenyl]-N-(2-phenylethyl)-1H- |
| | pyrazole-1-carboxamide |
| 1928 | 5-cyclopentyl-3-[2-hydroxy-5-(isobutyrylamino)phenyl]-N-(2-methylbenzyl)-1H- |
| | pyrazole-1-carboxamide |
| 1929 | 5-cyclopentyl-3-[2-hydroxy-5-(isobutyrylamino)phenyl]-N-(3-methylbenzyl)-1H- |
| | pyrazole-1-carboxamide |
| 1930 | 5-cyclopentyl-3-[2-hydroxy-5-(isobutyrylamino)phenyl]-N-(4-methylbenzyl)-1H- |
| | pyrazole-1-carboxamide |
| 1931 | N-(2-chlorobenzyl)-5-cyclopentyl-3-[2-hydroxy-5-(isobutyrylamino)phenyl]-1H- |
| | pyrazole-1-carboxamide |
| 1932 | 5-cyclopentyl-N-(4-fluorobenzyl)-3-[2-hydroxy-5-(isobutyrylamino)phenyl]-1H- |
| | pyrazole-1-carboxamide |
| 1933 | 5-cyclopentyl-N-(3,4-dichlorobenzyl)-3-[2-hydroxy-5-(isobutyrylamino)phenyl]- |
| | 1H-pyrazole-1-carboxamide |
| 1934 | 5-cyclopentyl-3-[2-hydroxy-5-(isobutyrylamino)phenyl]-N-(4-methoxybenzyl)- |
| | 1H-pyrazole-1-carboxamide |
| 1935 | N-(sec-butyl)-5-cyclopentyl-3-[2-hydroxy-5-(isobutyrylamino)phenyl]-1H- |
| | pyrazole-1-carboxamide |
| 1936 | 5-cyclopentyl-N-(2-fluorobenzyl)-3-[2-hydroxy-5-(isobutyrylamino)phenyl]-1H- |
| | pyrazole-1-carboxamide |
| 1937 | 5-cyclopentyl-N-(3-fluorobenzyl)-3-[2-hydroxy-5-(isobutyrylamino)phenyl]-1H- |
| | pyrazole-1-carboxamide |
| 1938 | 5-cyclopentyl-N-cyclopropyl-3-[2-hydroxy-5-(isobutyrylamino)phenyl]-1H- |
| | pyrazole-1-carboxamide |
| 1939 | 3-[5-(acetylamino)-2-hydroxyphenyl]-5-cyclopropyl-N-isopropyl-1H-pyrazole-1- |
| | carboxamide |
| 1940 | 3-[5-(acetylamino)-2-hydroxyphenyl]-5-cyclopropyl-N-ethyl-1H-pyrazole-1- |
| | carboxamide |
| 1941 | 3-[5-(acetylamino)-2-hydroxyphenyl]-N-allyl-5-cyclopropyl-1H-pyrazole-1- |
| | carboxamide |
| 1942 | 3-[5-(acetylamino)-2-hydroxyphenyl]-5-cyclopropyl-N-propyl-1H-pyrazole-1- |
| | carboxamide |
| 1943 | 3-[5-(acetylamino)-2-hydroxyphenyl]-N-benzyl-5-cyclopropyl-1H-pyrazole-1- |
| | carboxamide |
| 1944 | 3-[5-(acetylamino)-2-hydroxyphenyl]-5-cyclopropyl-N-(2-phenylethyl)-1H- |
| | pyrazole-1-carboxamide |
| 1945 | 3-[5-(acetylamino)-2-hydroxyphenyl]-5-cyclopropyl-N-(2-methylbenzyl)-1H- |
| | pyrazole-1-carboxamide |
| 1946 | 3-[5-(acetylamino)-2-hydroxyphenyl]-5-cyclopropyl-N-(3-methylbenzyl)-1H- |
| | pyrazole-1-carboxamide |
| 1947 | 3-[5-(acetylamino)-2-hydroxyphenyl]-5-cyclopropyl-N-(4-methylbenzyl)-1H- |
| | pyrazole-1-carboxamide |
| 1948 | 3-[5-(acetylamino)-2-hydroxyphenyl]-N-(2-chlorobenzyl)-5-cyclopropyl-1H- |
| | pyrazole-1-carboxamide |
| 1949 | 3-[5-(acetylamino)-2-hydroxyphenyl]-5-cyclopropyl-N-(4-fluorobenzyl)-1H- |
| | pyrazole-1-carboxamide |
| 1950 | 3-[5-(acetylamino)-2-hydroxyphenyl]-5-cyclopropyl-N-(3,4-dichlorobenzyl)-1H- |
| | pyrazole-1-carboxamide |
| 1951 | 3-[5-(acetylamino)-2-hydroxyphenyl]-5-cyclopropyl-N-(4-methoxybenzyl)-1H- |
| | pyrazole-1-carboxamide |
| 1952 | 3-[5-(acetylamino)-2-hydroxyphenyl]-N-(sec-butyl)-5-cyclopropyl-1H-pyrazole- |
| | 1-carboxamide |
| 1953 | 3-[5-(acetylamino)-2-hydroxyphenyl]-5-cyclopropyl-N-(2-fluorobenzyl)-1H- |
| | pyrazole-1-carboxamide |
| 1954 | 3-[5-(acetylamino)-2-hydroxyphenyl]-5-cyclopropyl-N-(3-fluorobenzyl)-1H- |
| | pyrazole-1-carboxamide |
| 1955 | 3-[5-(acetylamino)-2-hydroxyphenyl]-N,5-dicyclopropyl-1H-pyrazole-1- |
| | carboxamide |
| 1956 | 3-[5-(acetylamino)-2-hydroxyphenyl]-5-cyclobutyl-N-isopropyl-1H-pyrazole-1- |
| | carboxamide |
| 1957 | 3-[5-(acetylamino)-2-hydroxyphenyl]-5-cyclobutyl-N-ethyl-1H-pyrazole-1- |
| | carboxamide |
| 1958 | 3-[5-(acetylamino)-2-hydroxyphenyl]-N-allyl-5-cyclobutyl-1H-pyrazole-1- |
| | carboxamide |
| 1959 | 3-[5-(acetylamino)-2-hydroxyphenyl]-5-cyclobutyl-N-propyl-1H-pyrazole-1- |
| | carboxamide |
| 1960 | 3-[5-(acetylamino)-2-hydroxyphenyl]-N-benzyl-5-cyclobutyl-1H-pyrazole-1- |
| | carboxamide |
| 1961 | 3-[5-(acetylamino)-2-hydroxyphenyl]-5-cyclobutyl-N-(2-phenylethyl-1H- |
| | pyrazole-1-carboxamide |
| 1962 | 3-[5-(acetylamino)-2-hydroxyphenyl]-5-cyclobutyl-N-(2-methylbenzyl)-1H- |
| | pyrazole-1-carboxamide |
| 1963 | 3-[5-(acetylamino)-2-hydroxyphenyl]-5-cyclobutyl-N-(3-methylbenzyl)-1H- |
| | pyrazole-1-carboxamide |
| 1964 | 3-[5-(acetylamino)-2-hydroxyphenyl]-5-cyclobutyl-N-(4-methylbenzyl)-1H- |
| | pyrazole-1-carboxamide |
| 1965 | 3-[5-(acetylamino)-2-hydroxyphenyl]-N-(2-chlorobenzyl)-5-cyclobutyl-1H- |
| | pyrazole-1-carboxamide |
| 1966 | 3-[5-(acetylamino)-2-hydroxyphenyl]-5-cyclobutyl-N-(4-fluorobenzyl)-1H- |
| | pyrazole-1-carboxamide |
| 1967 | 3-[5-(acetylamino)-2-hydroxyphenyl]-5-cyclobutyl-N-(3,4-dichlorobenzyl)-1H- |
| | pyrazole-1-carboxamide |
| 1968 | 3-[5-(acetylamino)-2-hydroxyphenyl]-5-cyclobutyl-N-(4-methoxybenzyl)-1H- |
| | pyrazole-1-carboxamide |
| 1969 | 3-[5-(acetylamino)-2-hydroxyphenyl]-N-(sec-butyl)-5-cyclobutyl-1H-pyrazole-1- |
| | carboxamide |
| 1970 | 3-[5-(acetylamino)-2-hydroxyphenyl]-5-cyclobutyl-N-(2-fluorobenzyl)-1H- |
| | pyrazole-1-carboxamide |
| 1971 | 3-[5-(acetylamino)-2-hydroxyphenyl]-5-cyclobutyl-N-(3-fluorobenzyl)-1H- |
| | pyrazole-1-carboxamide |
| 1972 | 3-[5-(acetylamino)-2-hydroxyphenyl]-5-cyclobutyl-N-cyclopropyl-1H-pyrazole- |
| | 1-carboxamide |
| 1973 | 3-[5-(acetylamino)-2-hydroxyphenyl]-5-cyclopentyl-N-isopropyl-1H-pyrazole-1- |
| | carboxamide |
| 1974 | 3-[5-(acetylamino)-2-hydroxyphenyl]-5-cyclopentyl-N-ethyl-1H-pyrazole-1- |
| | carboxamide |
| 1975 | 3-[5-(acetylamino)-2-hydroxyphenyl]-N-allyl-5-cyclopentyl-1H-pyrazole-1- |
| | carboxamide |
| 1976 | 3-[5-(acetylamino)-2-hydroxyphenyl]-5-cyclopentyl-N-propyl-1H-pyrazole-1- |
| | carboxamide |
| 1977 | 3-[5-(acetylamino)-2-hydroxyphenyl]-N-benzyl-5-cyclopentyl-1H-pyrazole-1- |
| | carboxamide |
| 1978 | 3-[5-(acetylamino)-2-hydroxyphenyl]-5-cyclopentyl-N-(2-phenylethyl)-1H- |
| | pyrazole-1-carboxamide |
| 1979 | 3-[5-(acetylamino)-2-hydroxyphenyl]-5-cyclopentyl-N-(2-methylbenzyl)-1H- |
| | pyrazole-1-carboxamide |
| 1980 | 3-[5-(acetylamino)-2-hydroxyphenyl]-5-cyclopentyl-N-(3-methylbenzyl)-1H- |
| | pyrazole-1-carboxamide |
| 1981 | 3-[5-(acetylamino)-2-hydroxyphenyl]-5-cyclopentyl-N-(4-methylbenzyl)-1H- |
| | pyrazole-1-carboxamide |
| 1982 | 3-[5-(acetylamino)-2-hydroxyphenyl]-N-(2-chlorobenzyl)-5-cyclopentyl-1H- |
| | pyrazole-1-carboxamide |
| 1983 | 3-[5-(acetylamino)-2-hydroxyphenyl]-5-cyclopentyl-N-(4-fluorobenzyl)-1H- |
| | pyrazole-1-carboxamide |
| 1984 | 3-[5-(acetylamino)-2-hydroxyphenyl]-5-cyclopentyl-N-(3,4-dichlorobenzyl)-1H- |
| | pyrazole-1-carboxamide |
| 1985 | 3-[5-(acetylamino)-2-hydroxyphenyl]-5-cyclopentyl-N-(4-methoxybenzyl)-1H- |
| | pyrazole-1-carboxamide |
| 1986 | 3-[5-(acetylamino)-2-hydroxyphenyl]-N-(sec-butyl)-5-cyclopentyl-1H-pyrazole- |
| | 1-carboxamide |
| 1987 | 3-[5-(acetylamino)-2-hydroxyphenyl]-5-cyclopentyl-N-(2-fluorobenzyl)-1H- |
| | pyrazole-1-carboxamide |
| 1988 | 3-[5-(acetylamino)-2-hydroxyphenyl]-5-cyclopentyl-N-(3-fluorobenzyl)-1H- |
| | pyrazole-1-carboxamide |
| 1989 | 3-[5-(acetylamino)-2-hydroxyphenyl]-5-cyclopentyl-N-cyclopropyl-1H-pyrazole- |
| | 1-carboxamide |
| 1990 | 5-cyclopropyl-3-{2-hydroxy-5-[(3-methylbutanoyl)amino]phenyl}-N-isopropyl- |
| | 1H-pyrazole-1-carboxamide |
| 1991 | 5-cyclopropyl-N-ethyl-3-{2-hydroxy-5-[(3-methylbutanoyl)amino]phenyl}-1H- |
| | pyrazole-1-carboxamide |
| 1992 | N-allyl-5-cyclopropyl-3-{2-hydroxy-5-[(3-methylbutanoyl)amino]phenyl}-1H- |
| | pyrazole-1-carboxamide |
| 1993 | 5-cyclopropyl-3-{2-hydroxy-5-[(3-methylbutanoyl)amino]phenyl}-N-propyl-1H- |
| | pyrazole-1-carboxamide |
| 1994 | N-benzyl-5-cyclopropyl-3-{2-hydroxy-5-[(3-methylbutanoyl)amino]phenyl}-1H- |
| | pyrazole-1-carboxamide |
| 1995 | 5-cyclopropyl-3-{2-hydroxy-5-[(3-methylbutanoyl)amino]phenyl}-N-(2- |
| | phenylethyl)-1H-pyrazole-1-carboxamide |
| 1996 | 5-cyclopropyl-3-{2-hydroxy-5-[(3-methylbutanoyl)amino]phenyl}-N-(2- |
| | methylbenzyl)-1H-pyrazole-1-carboxamide |
| 1997 | 5-cyclopropyl-3-{2-hydroxy-5-[(3-methylbutanoyl)amino]phenyl}-N-(3- |
| | methylbenzyl)-1H-pyrazole-1-carboxamide |
| 1998 | 5-cyclopropyl-3-{2-hydroxy-5-[(3-methylbutanoyl)amino]phenyl}-N-(4- |
| | methylbenzyl)-1H-pyrazole-1-carboxamide |
| 1999 | N-(2-chlorobenzyl)-5-cyclopropyl-3-{2-hydroxy-5-[(3- |
| | methylbutanoyl)amino]phenyl}-1H-pyrazole-1-carboxamide |
| 2000 | 5-cyclopropyl-N-(4-fluorobenzyl)-3-{2-hydroxy-5-[(3- |
| | methylbutanoyl)amino]phenyl}-1H-pyrazole-1-carboxamide |
| 2001 | 5-cyclopropyl-N-(3,4-dichlorobenzyl)-3-{2-hydroxy-5-[(3- |
| | methylbutanoyl)amino]phenyl}-1H-pyrazole-1-carboxamide |
| 2002 | 5-cyclopropyl-3-{2-hydroxy-5-[(3-methylbutanoyl)amino]phenyl}-N-(4- |
| | methoxybenzyl)-1H-pyrazole-1-carboxamide |
| 2003 | N-(sec-butyl)-5-cyclopropyl-3-{2-hydroxy-5-[(3-methylbutanoyl)amino]phenyl}- |
| | 1H-pyrazole-1-carboxamide |
| 2004 | 5-cyclopropyl-N-(2-fluorobenzyl)-3-{2-hydroxy-5-[(3- |
| | methylbutanoyl)amino]phenyl}-1H-pyrazole-1-carboxamide |
| 2005 | 5-cyclopropyl-N-(3-fluorobenzyl)-3-{2-hydroxy-5-[(3- |
| | methylbutanoyl)amino]phenyl}-1H-pyrazole-1-carboxamide |
| 2006 | N,5-dicyclopropyl-3-{2-hydroxy-5-[(3-methylbutanoyl)amino]phenyl}-1H- |
| | pyrazole-1-carboxamide |
| 2007 | 5-cyclobutyl-3-{2-hydroxy-5-[(3-methylbutanoyl)amino]phenyl}-N-isopropyl- |
| | 1H-pyrazole-1-carboxamide |
| 2008 | 5-cyclobutyl-N-ethyl-3-{2-hydroxy-5-[(3-methylbutanoyl)amino]phenyl}-1H- |
| | pyrazole-1-carboxamide |
| 2009 | N-allyl-5-cyclobutyl-3-{2-hydroxy-5-[(3-methylbutanoyl)amino]phenyl}-1H- |
| | pyrazole-1-carboxamide |
| 2010 | 5-cyclobutyl-3-{2-hydroxy-5-[(3-methylbutanoyl)amino]phenyl}-N-propyl-1H- |
| | pyrazole-1-carboxamide |
| 2011 | N-benzyl-5-cyclobutyl-3-{2-hydroxy-5-[(3-methylbutanoyl)amino]phenyl}-1H- |
| | pyrazole-1-carboxamide |
| 2012 | 5-cyclobutyl-3-{2-hydroxy-5-[(3-methylbutanoyl)amino]phenyl}-N-(2- |
| | phenylethyl)-1H-pyrazole-1-carboxamide |
| 2013 | 5-cyclobutyl-3-{2-hydroxy-5-[(3-methylbutanoyl)amino]phenyl}-N-(2- |
| | methylbenzyl)-1H-pyrazole-1-carboxamide |
| 2014 | 5-cyclobutyl-3-{2-hydroxy-5-[(3-methylbutanoyl)amino]phenyl}-N-(3- |
| | methylbenzyl)-1H-pyrazole-1-carboxamide |
| 2015 | 5-cyclobutyl-3-{2-hydroxy-5-[(3-methylbutanoyl)amino]phenyl}-N-(4- |
| | methylbenzyl)-1H-pyrazole-1-carboxamide |
| 2016 | N-(2-chlorobenzyl)-5-cyclobutyl-3-{2-hydroxy-5-[(3- |
| | methylbutanoyl)amino]phenyl}-1H-pyrazole-1-carboxamide |
| 2017 | 5-cyclobutyl-N-(4-fluorobenzyl)-3-{2-hydroxy-5-[(3- |
| | methylbutanoyl)amino]phenyl}-1H-pyrazole-1-carboxamide |
| 2018 | 5-cyclobutyl-N-(3,4-dichlorobenzyl)-3-{2-hydroxy-5-[(3- |
| | methylbutanoyl)amino]phenyl}-1H-pyrazole-1-carboxamide |
| 2019 | 5-cyclobutyl-3-{2-hydroxy-5-[(3-methylbutanoyl)amino]phenyl}-N-(4- |
| | methoxybenzyl)-1H-pyrazole-1-carboxamide |
| 2020 | N-(sec-butyl)-5-cyclobutyl-3-{2-hydroxy-5-[(3-methylbutanoyl)amino]phenyl}- |
| | 1H-pyrazole-1-carboxamide |
| 2021 | 5-cyclobutyl-N-(2-fluorobenzyl)-3-{2-hydroxy-5-[(3- |
| | methylbutanoyl)amino]phenyl}-1H-pyrazole-1-carboxamide |
| 2022 | 5-cyclobutyl-N-(3-fluorobenzyl)-3-{2-hydroxy-5-[(3- |
| | methylbutanoyl)amino]phenyl}-1H-pyrazole-1-carboxamide |
| 2023 | 5-cyclobutyl-N-cyclopropyl-3-{2-hydroxy-5-[(3-methylbutanoyl)amino]phenyl}- |
| | 1H-pyrazole-1-carboxamide |
| 2024 | 5-cyclopentyl-3-{2-hydroxy-5-[(3-methylbutanoyl)amino]phenyl}-N-isopropyl- |
| | 1H-pyrazole-1-carboxamide |
| 2025 | 5-cyclopentyl-N-ethyl-3-{2-hydroxy-5-[(3-methylbutanoyl)amino]phenyl}-1H- |
| | pyrazole-1-carboxamide |
| 2026 | N-allyl-5-cyclopentyl-3-{2-hydroxy-5-[(3-methylbutanoyl)amino]phenyl}-1H- |
| | pyrazole-1-carboxamide |
| 2027 | 5-cyclopentyl-3-{2-hydroxy-5-[(3-methylbutanoyl)amino]phenyl}-N-propyl-1H- |
| | pyrazole-1-carboxamide |
| 2028 | N-benzyl-5-cyclopentyl-3-{2-hydroxy-5-[(3-methylbutanoyl)amino]phenyl}-1H- |
| | pyrazole-1-carboxamide |
| 2029 | 5-cyclopentyl-3-{2-hydroxy-5-[(3-methylbutanoyl)amino]phenyl}-N-(2- |
| | phenylethyl)-1H-pyrazole-1-carboxamide |
| 2030 | 5-cyclopentyl-3-{2-hydroxy-5-[(3-methylbutanoyl)amino]phenyl}-N-(2- |
| | methylbenzyl)-1H-pyrazole-1-carboxamide |
| 2031 | 5-cyclopentyl-3-{2-hydroxy-5-[(3-methylbutanoyl)amino]phenyl}-N-(3- |
| | methylbenzyl)-1H-pyrazole-1-carboxamide |
| 2032 | 5-cyclopentyl-3-{2-hydroxy-5-[(3-methylbutanoyl)amino]phenyl}-N-(4- |
| | methylbenzyl)-1H-pyrazole-1-carboxamide |
| 2033 | N-(2-chlorobenzyl)-5-cyclopentyl-3-{2-hydroxy-5-[(3- |
| | methylbutanoyl)amino]phenyl}-1H-pyrazole-1-carboxamide |
| 2034 | 5-cyclopentyl-N-(4-fluorobenzyl)-3-{2-hydroxy-5-[(3- |
| | methylbutanoyl)amino]phenyl}-1H-pyrazole-1-carboxamide |
| 2035 | 5-cyclopentyl-N-(3,4-dichlorobenzyl)-3-{2-hydroxy-5-[(3- |
| | methylbutanoyl)amino]phenyl}-1H-pyrazole-1-carboxamide |
| 2036 | 5-cyclopentyl-3-{2-hydroxy-5-[(3-methylbutanoyl)amino]phenyl}-N-(4- |
| | methoxybenzyl)-1H-pyrazole-1-carboxamide |
| 2037 | N-(sec-butyl)-5-cyclopentyl-3-{2-hydroxy-5-[(3-methylbutanoyl)amino]phenyl}- |
| | 1H-pyrazole-1-carboxamide |
| 2038 | 5-cyclopentyl-N-(2-fluorobenzyl)-3-{2-hydroxy-5-[(3- |
| | methylbutanoyl)amino]phenyl}-1H-pyrazole-1-carboxamide |
| 2039 | 5-cyclopentyl-N-(3-fluorobenzyl)-3-{2-hydroxy-5-[(3- |
| | methylbutanoyl)amino]phenyl}-1H-pyrazole-1-carboxamide |
| 2040 | 5-cyclopentyl-N-cyclopropyl-3-{2-hydroxy-5-[(3- |
| | methylbutanoyl)amino]phenyl}-1H-pyrazole-1-carboxamide |
| 2041 | 5-cyclopropyl-3-[2-hydroxy-5-(propionylamino)phenyl]-N-isopropyl-1H- |
| | pyrazole-1-carboxamide |
| 2042 | 5-cyclopropyl-N-ethyl-3-[2-hydroxy-5-(propionylamino)phenyl]-1H-pyrazole-1- |
| | carboxamide |
| 2043 | N-allyl-5-cyclopropyl-3-[2-hydroxy-5-(propionylamino)phenyl]-1H-pyrazole-1- |
| | carboxamide |
| 2044 | 5-cyclopropyl-3-[2-hydroxy-5-(propionylamino)phenyl]-N-propyl-1H-pyrazole-1- |
| | carboxamide |
| 2045 | N-benzyl-5-cyclopropyl-3-[2-hydroxy-5-(propionylamino)phenyl]-1H-pyrazole- |
| | 1-carboxamide |
| 2046 | 5-cyclopropyl-3-[2-hydroxy-5-(propionylamino)phenyl]-N-(2-phenylethyl)-1H- |
| | pyrazole-1-carboxamide |
| 2047 | 5-cyclopropyl-3-[2-hydroxy-5-(propionylamino)phenyl]-N-(2-methylbenzyl)-1H- |
| | pyrazole-1-carboxamide |
| 2048 | 5-cyclopropyl-3-[2-hydroxy-5-(propionylamino)phenyl]-N-(3-methylbenzyl)-1H- |
| | pyrazole-1-carboxamide |
| 2049 | 5-cyclopropyl-3-[2-hydroxy-5-(propionylamino)phenyl]-N-(4-methylbenzyl)-1H- |
| | pyrazole-1-carboxamide |
| 2050 | N-(2-chlorobenzyl)-5-cyclopropyl-3-[2-hydroxy-5-(propionylamino)phenyl]-1H- |
| | pyrazole-1-carboxamide |
| 2051 | 5-cyclopropyl-N-(4-fluorobenzyl)-3-[2-hydroxy-5-(propionylamino)phenyl]-1H- |
| | pyrazole-1-carboxamide |
| 2052 | 5-cyclopropyl-N-(3,4-dichlorobenzyl)-3-[2-hydroxy-5-(propionylamino)phenyl]- |
| | 1H-pyrazole-1-carboxamide |
| 2053 | 5-cyclopropyl-3-[2-hydroxy-5-(propionylamino)phenyl]-N-(4-methoxybenzyl)- |
| | 1H-pyrazole-1-carboxamide |
| 2054 | N-(sec-butyl)-5-cyclopropyl-3-[2-hydroxy-5-(propionylamino)phenyl]-1H- |
| | pyrazole-1-carboxamide |
| 2055 | 5-cyclopropyl-N-(2-fluorobenzyl)-3-[2-hydroxy-5-(propionylamino)phenyl]-1H- |
| | pyrazole-1-carboxamide |
| 2056 | 5-cyclopropyl-N-(3-fluorobenzyl)-3-[2-hydroxy-5-(propionylamino)phenyl]-1H- |
| | pyrazole-1-carboxamide |
| 2057 | N,5-dicyclopropyl-3-[2-hydroxy-5-(propionylamino)phenyl]-1H-pyrazole-1- |
| | carboxamide |
| 2058 | 5-cyclobutyl-3-[2-hydroxy-5-(propionylamino)phenyl]-N-isopropyl-1H-pyrazole- |
| | 1-carboxamide |
| 2059 | 5-cyclobutyl-N-ethyl-3-[2-hydroxy-5-(propionylamino)phenyl]-1H-pyrazole-1- |
| | carboxamide |
| 2060 | N-allyl-5-cyclobutyl-3-[2-hydroxy-5-(propionylamino)phenyl]-1H-pyrazole-1- |
| | carboxamide |
| 2061 | 5-cyclobutyl-3-[2-hydroxy-5-(propionylamino)phenyl]-N-propyl-1H-pyrazole-1- |
| | carboxamide |
| 2062 | N-benzyl-5-cyclobutyl-3-[2-hydroxy-5-(propionylamino)phenyl]-1H-pyrazole-1- |
| | carboxamide |
| 2063 | 5-cyclobutyl-3-[2-hydroxy-5-(propionylamino)phenyl]-N-(2-phenylethyl)-1H- |
| | pyrazole-1-carboxamide |
| 2064 | 5-cyclobutyl-3-[2-hydroxy-5-(propionylamino)phenyl]-N-(2-methylbenzyl)-1H- |
| | pyrazole-1-carboxamide |
| 2065 | 5-cyclobutyl-3-[2-hydroxy-5-(propionylamino)phenyl]-N-(3-methylbenzyl)-1H- |
| | pyrazole-1-carboxamide |
| 2066 | 5-cyclobutyl-3-[2-hydroxy-5-(propionylamino)phenyl]-N-(4-methylbenzyl)-1H- |
| | pyrazole-1-carboxamide |
| 2067 | N-(2-chlorobenzyl)-5-cyclobutyl-3-[2-hydroxy-5-(propionylamino)phenyl]-1H- |
| | pyrazole-1-carboxamide |
| 2068 | 5-cyclobutyl-N-(4-fluorobenzyl)-3-[2-hydroxy-5-(propionylamino)phenyl]-1H- |
| | pyrazole-1-carboxamide |
| 2069 | 5-cyclobutyl-N-(3,4-dichlorobenzyl)-3-[2-hydroxy-5-(propionylamino)phenyl]- |
| | 1H-pyrazole-1-carboxamide |
| 2070 | 5-cyclobutyl-3-[2-hydroxy-5-(propionylamino)phenyl]-N-(4-methoxybenzyl)-1H |
| | pyrazole-1-carboxamide |
| 2071 | N-(sec-butyl)-5-cyclobutyl-3-[2-hydroxy-5-(propionylamino)phenyl]-1H- |
| | pyrazole-1-carboxamide |
| 2072 | 5-cyclobutyl-N-(2-fluorobenzyl)-3-[2-hydroxy-5-(propionylamino)phenyl]-1H- |
| | pyrazole-1-carboxamide |
| 2073 | 5-cyclobutyl-N-(3-fluorobenzyl)-3-[2-hydroxy-5-(propionylamino)phenyl]-1H- |
| | pyrazole-1-carboxamide |
| 2074 | 5-cyclobutyl-N-cyclopropyl-3-[2-hydroxy-5-(propionylamino)phenyl]-1H- |
| | pyrazole-1-carboxamide |
| 2075 | 5-cyclopentyl-3-[2-hydroxy-5-(propionylamino)phenyl]-N-isopropyl-1H- |
| | pyrazole-1-carboxamide |
| 2076 | 5-cyclopentyl-N-ethyl-3-[2-hydroxy-5-(propionylamino)phenyl]-1H-pyrazole-1- |
| | carboxamide |
| 2077 | N-allyl-5-cyclopentyl-3-[2-hydroxy-5-(propionylamino)phenyl]-1H-pyrazole-1- |
| | carboxamide |
| 2078 | 5-cyclopentyl-3-[2-hydroxy-5-(propionylamino)phenyl]-N-propyl-1H-pyrazole-1- |
| | carboxamide |
| 2079 | N-benzyl-5-cyclopentyl-3-[2-hydroxy-5-(propionylamino)phenyl]-1H-pyrazole-1- |
| | carboxamide |
| 2080 | 5-cyclopentyl-3-[2-hydroxy-5-(propionylamino)phenyl]-N-(2-phenylethyl)-1H- |
| | pyrazole-1-carboxamide |
| 2081 | 5-cyclopentyl-3-[2-hydroxy-5-(propionylamino)phenyl]-N-(2-methylbenzyl)-1H- |
| | pyrazole-1-carboxamide |
| 2082 | 5-cyclopentyl-3-[2-hydroxy-5-(propionylamino)phenyl]-N-(3-methylbenzyl)-1H- |
| | pyrazole-1-carboxamide |
| 2083 | 5-cyclopentyl-3-[2-hydroxy-5-(propionylamino)phenyl]-N-(4-methylbenzyl)-1H- |
| | pyrazole-1-carboxamide |
| 2084 | N-(2-chlorobenzyl)-5-cyclopentyl-3-[2-hydroxy-5-(propionylamino)phenyl]-1H- |
| | pyrazole-1-carboxamide |
| 2085 | 5-cyclopentyl-N-(4-fluorobenzyl)-3-[2-hydroxy-5-(propionylamino)phenyl]-1H- |
| | pyrazole-1-carboxamide |
| 2086 | 5-cyclopentyl-N-(3,4-dichlorobenzyl)-3-[2-hydroxy-5-(propionylamino)phenyl]- |
| | 1H-pyrazole-1-carboxamide |
| 2087 | 5-cyclopentyl-3-[2-hydroxy-5-(propionylamino)phenyl]-N-(4-methoxybenzyl)- |
| | 1H-pyrazole-1-carboxamide |
| 2088 | N-(sec-butyl)-5-cyclopentyl-3-[2-hydroxy-5-(propionylamino)phenyl]-1H- |
| | pyrazole-1-carboxamide |
| 2089 | 5-cyclopentyl-N-(2-fluorobenzyl)-3-[2-hydroxy-5-(propionylamino)phenyl]-1H- |
| | pyrazole-1-carboxamide |
| 2090 | 5-cyclopentyl-N-(3-fluorobenzyl)-3-[2-hydroxy-5-(propionylamino)phenyl]-1H- |
| | pyrazole-1-carboxamide |
| 2091 | 5-cyclopentyl-N-cyclopropyl-3-[2-hydroxy-5-(propionylamino)phenyl]-1H- |
| | pyrazole-1-carboxamide |
| 2092 | 5-cyclopropyl-3-{5-[(cyclopropylcarbonyl)amino]-2-hydroxyphenyl}-N- |
| | isopropyl-1H-pyrazole-1-carboxamide |
| 2093 | 5-cyclopropyl-3-{5-[(cyclopropylcarbonyl)amino]-2-hydroxyphenyl}-N-ethyl- |
| | 1H-pyrazole-1-carboxamide |
| 2094 | N-allyl-5-cyclopropyl-3-{5-[(cyclopropylcarbonyl)amino]-2-hydroxyphenyl}-1H- |
| | pyrazole-1-carboxamide |
| 2095 | 5-cyclopropyl-3-{5-[(cyclopropylcarbonyl)amino]-2-hydroxyphenyl}-N-propyl- |
| | 1H-pyrazole-1-carboxamide |
| 2096 | N-benzyl-5-cyclopropyl-3-{5-[(cyclopropylcarbonyl)amino]-2-hydroxyphenyl}- |
| | 1H-pyrazole-1-carboxamide |
| 2097 | 5-cyclopropyl-3-{5-[(cyclopropylcarbonyl)amino]-2-hydroxyphenyl}-N-(2- |
| | phenylethyl)-1H-pyrazole-1-carboxamide |
| 2098 | 5-cyclopropyl-3-{5-[(cyclopropylcarbonyl)amino]-2-hydroxyphenyl}-N-(2- |
| | methylbenzyl)-1H-pyrazole-1-carboxamide |
| 2099 | 5-cyclopropyl-3-{5-[(cyclopropylcarbonyl)amino]-2-hydroxyphenyl}-N-(3- |
| | methylbenzyl)-1H-pyrazole-1-carboxamide |
| 2100 | 5-cyclopropyl-3-{5-[(cyclopropylcarbonyl)amino]-2-hydroxyphenyl}-N-(4- |
| | methylbenzyl)-1H-pyrazole-1-carboxamide |
| 2101 | N-(2-chlorobenzyl)-5-cyclopropyl-3-{5-[(cyclopropylcarbonyl)amino]-2- |
| | hydroxyphenyl}-1H-pyrazole-1-carboxamide |
| 2102 | 5-cyclopropyl-3-{5-[(cyclopropylcarbonyl)amino]-2-hydroxyphenyl}-N-(4- |
| | fluorobenzyl)-1H-pyrazole-1-carboxamide |
| 2103 | 5-cyclopropyl-3-{5-[(cyclopropylcarbonyl)amino]-2-hydroxyphenyl}-N-(3,4- |
| | dichlorobenzyl)-1H-pyrazole-1-carboxamide |
| 2104 | 5-cyclopropyl-3-{5-[(cyclopropylcarbonyl)amino]-2-hydroxyphenyl}-N-(4- |
| | methoxybenzyl)-1H-pyrazole-1-carboxamide |
| 2105 | N-(sec-butyl)-5-cyclopropyl-3-{5-[(cyclopropylcarbonyl)amino]-2- |
| | hydroxyphenyl}-1H-pyrazole-1-carboxamide |
| 2106 | 5-cyclopropyl-3-{5-[(cyclopropylcarbonyl)amino]-2-hydroxyphenyl}-N-(2- |
| | fluorobenzyl)-1H-pyrazole-1-carboxamide |
| 2107 | 5-cyclopropyl-3-{5-[(cyclopropylcarbonyl)amino]-2-hydroxyphenyl}-N-(3- |
| | fluorobenzyl)-1H-pyrazole-1-carboxamide |
| 2108 | N,5-dicyclopropyl-3-{5-[(cyclopropylcarbonyl)amino]-2-hydroxyphenyl}-1H- |
| | pyrazole-1-carboxamide |
| 2109 | 5-cyclobutyl-3-{5-[(cyclopropylcarbonyl)amino]-2-hydroxyphenyl}-N-isopropyl- |
| | 1H-pyrazole-1-carboxamide |
| 2110 | 5-cyclobutyl-3-{5-[(cyclopropylcarbonyl)amino]-2-hydroxyphenyl}-N-ethyl-1H- |
| | pyrazole-1-carboxamide |
| 2111 | N-allyl-5-cyclobutyl-3-{5-[(cyclopropylcarbonyl)amino]-2-hydroxyphenyl}-1H- |
| | pyrazole-1-carboxamide |
| 2112 | 5-cyclobutyl-3-{5-[(cyclopropylcarbonyl)amino]-2-hydroxyphenyl}-N-propyl- |
| | 1H-pyrazole-1-carboxamide |
| 2113 | N-benzyl-5-cyclobutyl-3-{5-[(cyclopropylcarbonyl)amino]-2-hydroxyphenyl}- |
| | 1H-pyrazole-1-carboxamide |
| 2114 | 5-cyclobutyl-3-{5-[(cyclopropylcarbonyl)amino]-2-hydroxyphenyl}-N-(2- |
| | phenylethyl)-1H-pyrazole-1-carboxamide |
| 2115 | 5-cyclobutyl-3-{5-[(cyclopropylcarbonyl)amino]-2-hydroxyphenyl}-N-(2- |
| | methylbenzyl)-1H-pyrazole-1-carboxamide |
| 2116 | 5-cyclobutyl-3-{5-[(cyclopropylcarbonyl)amino]-2-hydroxyphenyl}-N-(3- |
| | methylbenzyl)-1H-pyrazole-1-carboxamide |
| 2117 | 5-cyclobutyl-3-{5-[(cyclopropylcarbonyl)amino]-2-hydroxyphenyl}-N-(4- |
| | methylbenzyl)-1H-pyrazole-1-carboxamide |
| 2118 | N-(2-chlorobenzyl)-5-cyclobutyl-3-{5-[(cyclopropylcarbonyl)amino]-2- |
| | hydroxyphenyl}-1H-pyrazole-1-carboxamide |
| 2119 | 5-cyclobutyl-3-{5-[(cyclopropylcarbonyl)amino]-2-hydroxyphenyl}-N-(4- |
| | fluorobenzyl)-1H-pyrazole-1-carboxamide |
| 2120 | 5-cyclobutyl-3-{5-[(cyclopropylcarbonyl)amino]-2-hydroxyphenyl}-N-(3,4- |
| | dichlorobenzyl)-1H-pyrazole-1-carboxamide |
| 2121 | 5-cyclobutyl-3-{5-[(cyclopropylcarbonyl)amino]-2-hydroxyphenyl}-N-(4- |
| | methoxybenzyl)-1H-pyrazole-1-carboxamide |
| 2122 | N-(sec-butyl)-5-cyclobutyl-3-{5-[(cyclopropylcarbonyl)amino]-2- |
| | hydroxyphenyl}-1H-pyrazole-1-carboxamide |
| 2123 | 5-cyclobutyl-3-{5-[(cyclopropylcarbonyl)amino]-2-hydroxyphenyl}-N-(2- |
| | fluorobenzyl)-1H-pyrazole-1-carboxamide |
| 2124 | 5-cyclobutyl-3-{5-[(cyclopropylcarbonyl)amino]-2-hydroxyphenyl}-N-(3- |
| | fluorobenzyl)-1H-pyrazole-1-carboxamide |
| 2125 | 5-cyclobutyl-N-cyclopropyl-3-{5-[(cyclopropylcarbonyl)amino]-2- |
| | hydroxyphenyl}-1H-pyrazole-1-carboxamide |
| 2126 | 5-cyclopentyl-3-{5-[(cyclopropylcarbonyl)amino]-2-hydroxyphenyl}-N- |
| | isopropyl-1H-pyrazole-1-carboxamide |
| 2127 | 5-cyclopentyl-3-{5-[(cyclopropylcarbonyl)amino]-2-hydroxyphenyl}-N-ethyl- |
| | 1H-pyrazole-1-carboxamide |
| 2128 | N-allyl-5-cyclopentyl-3-{5-[(cyclopropylcarbonyl)amino]-2-hydroxyphenyl}-1H- |
| | pyrazole-1-carboxamide |
| 2129 | 5-cyclopentyl-3-{5-[(cyclopropylcarbonyl)amino]-2-hydroxyphenyl}-N-propyl- |
| | 1H-pyrazole-1-carboxamide |
| 2130 | N-benzyl-5-cyclopentyl-3-{5-[(cyclopropylcarbonyl)amino]-2-hydroxyphenyl}- |
| | 1H-pyrazole-1-carboxamide |
| 2131 | 5-cyclopentyl-3-{5-[(cyclopropylcarbonyl)amino]-2-hydroxyphenyl}-N-(2- |
| | phenylethyl)-1H-pyrazole-1-carboxamide |
| 2132 | 5-cyclopentyl-3-{5-[(cyclopropylcarbonyl)amino]-2-hydroxyphenyl}-N-(2- |
| | methylbenzyl)-1H-pyrazole-1-carboxamide |
| 2133 | 5-cyclopentyl-3-{5-[(cyclopropylcarbonyl)amino]-2-hydroxyphenyl}-N-(3- |
| | methylbenzyl)-1H-pyrazole-1-carboxamide |
| 2134 | 5-cyclopentyl-3-{5-[(cyclopropylcarbonyl)amino]-2-hydroxyphenyl}-N-(4- |
| | methylbenzyl)-1H-pyrazole-1-carboxamide |
| 2135 | N-(2-chlorobenzyl)-5-cyclopentyl-3-{5-[(cyclopropylcarbonyl)amino]-2- |
| | hydroxyphenyl}-1H-pyrazole-1-carboxamide |
| 2136 | 5-cyclopentyl-3-{5-[(cyclopropylcarbonyl)amino]-2-hydroxyphenyl}-N-(4- |
| | fluorobenzyl)-1H-pyrazole-1-carboxamide |
| 2137 | 5-cyclopentyl-3-{5-[(cyclopropylcarbonyl)amino]-2-hydroxyphenyl}-N-(3,4- |
| | dichlorobenzyl)-1H-pyrazole-1-carboxamide |
| 2138 | 5-cyclopentyl-3-{5-[(cyclopropylcarbonyl)amino]-2-hydroxyphenyl}-N-(4- |
| | methoxybenzyl)-1H-pyrazole-1-carboxamide |
| 2139 | N-(sec-butyl)-5-cyclopentyl-3-{5-[(cyclopropylcarbonyl)amino]-2- |
| | hydroxyphenyl}-1H-pyrazole-1-carboxamide |
| 2140 | 5-cyclopentyl-3-{5-[(cyclopropylcarbonyl)amino]-2-hydroxyphenyl}-N-(2- |
| | fluorobenzyl)-1H-pyrazole-1-carboxamide |
| 2141 | 5-cyclopentyl-3-{5-[(cyclopropylcarbonyl)amino]-2-hydroxyphenyl}-N-(3- |
| | fluorobenzyl)-1H-pyrazole-1-carboxamide |
| 2142 | 5-cyclopentyl-N-cyclopropyl-3-{5-[(cyclopropylcarbonyl)amino]-2- |
| | hydroxyphenyl}-1H-pyrazole-1-carboxamide |
| 2143 | 5-cyclopropyl-3-[5-(2-furoylamino)-2-hydroxyphenyl]-N-isopropyl-1H-pyrazole- |
| | 1-carboxamide |
| 2144 | 5-cyclopropyl-N-ethyl-3-[5-(2-furoylamino)-2-hydroxyphenyl]-1H-pyrazole-1- |
| | carboxamide |
| 2145 | N-allyl-5-cyclopropyl-3-[5-(2-furoylamino)-2-hydroxyphenyl]-1H-pyrazole-1- |
| | carboxamide |
| 2146 | 5-cyclopropyl-3-[5-(2-furoylamino)-2-hydroxyphenyl]-N-propyl-1H-pyrazole-1- |
| | carboxamide |
| 2147 | N-benzyl-5-cyclopropyl-3-[5-(2-furoylamino)-2-hydroxyphenyl]-1H-pyrazole-1- |
| | carboxamide |
| 2148 | 5-cyclopropyl-3-[5-(2-furoylamino)-2-hydroxyphenyl]-N-(2-phenylethyl)-1H- |
| | pyrazole-1-carboxamide |
| 2149 | 5-cyclopropyl-3-[5-(2-furoylamino)-2-hydroxyphenyl]-N-(2-methylbenzyl)-1H- |
| | pyrazole-1-carboxamide |
| 2150 | 5-cyclopropyl-3-[5-(2-furoylamino)-2-hydroxyphenyl]-N-(3-methylbenzyl)-1H- |
| | pyrazole-1-carboxamide |
| 2151 | 5-cyclopropyl-3-[5-(2-furoylamino)-2-hydroxyphenyl]-N-(4-methylbenzyl)-1H- |
| | pyrazole-1-carboxamide |
| 2152 | N-(2-chlorobenzyl)-5-cyclopropyl-3-[5-(2-furoylamino)-2-hydroxyphenyl]-1H- |
| | pyrazole-1-carboxamide |
| 2153 | 5-cyclopropyl-N-(4-fluorobenzyl)-3-[5-(2-furoylamino)-2-hydroxyphenyl]-1H- |
| | pyrazole-1-carboxamide |
| 2154 | 5-cyclopropyl-N-(3,4-dichlorobenzyl)-3-[5-(2-furoylamino)-2-hydroxyphenyl]- |
| | 1H-pyrazole-1-carboxamide |
| 2155 | 5-cyclopropyl-3-[5-(2-furoylamino)-2-hydroxyphenyl]-N-(4-methoxybenzyl)-1H- |
| | pyrazole-1-carboxamide |
| 2156 | N-(sec-butyl)-5-cyclopropyl-3-[5-(2-furoylamino)-2-hydroxyphenyl]-1H- |
| | pyrazole-1-carboxamide |
| 2157 | 5-cyclopropyl-N-(2-fluorobenzyl)-3-[5-(2-furoylamino)-2-hydroxyphenyl]-1H- |
| | pyrazole-1-carboxamide |
| 2158 | 5-cyclopropyl-N-(3-fluorobenzyl)-3-[5-(2-furoylamino)-2-hydroxyphenyl]-1H- |
| | pyrazole-1-carboxamide |
| 2159 | N,5-dicyclopropyl-3-[5-(2-furoylamino)-2-hydroxyphenyl]-1H-pyrazole-1- |
| | carboxamide |
| 2160 | 5-cyclobutyl-3-[5-(2-furoylamino)-2-hydroxyphenyl]-N-isopropyl-1H-pyrazole-1- |
| | carboxamide |
| 2161 | 5-cyclobutyl-N-ethyl-3-[5-(2-furoylamino)-2-hydroxyphenyl]-1H-pyrazole-1- |
| | carboxamide |
| 2162 | N-allyl-5-cyclobutyl-3-[5-(2-furoylamino)-2-hydroxyphenyl]-1H-pyrazole-1- |
| | carboxamide |
| 2163 | 5-cyclobutyl-3-[5-(2-furoylamino)-2-hydroxyphenyl]-N-propyl-1H-pyrazole-1- |
| | carboxamide |
| 2164 | N-benzyl-5-cyclobutyl-3 -[5-(2-furoylamino)-2-hydroxyphenyl]-1H-pyrazole-1- |
| | carboxamide |
| 2165 | 5-cyclobutyl-3-[5-(2-furoylamino)-2-hydroxyphenyl]-N-(2-phenylethyl)-1H- |
| | pyrazole-1-carboxamide |
| 2166 | 5-cyclobutyl-3-[5-(2-furoylamino)-2-hydroxyphenyl]-N-(2-methylbenzyl)-1H- |
| | pyrazole-1-carboxamide |
| 2167 | 5-cyclobutyl-3-[5-(2-furoylamino)-2-hydroxyphenyl]-N-(3-methylbenzyl)-1H- |
| | pyrazole-1-carboxamide |
| 2168 | 5-cyclobutyl-3-[5-(2-furoylamino)-2-hydroxyphenyl]-N(4-methylbenzyl)-1H- |
| | pyrazole-1-carboxamide |
| 2169 | N-(2-chlorobenzyl)-5-cyclobutyl-3-[5-(2-furoylamino)-2-hydroxyphenyl]-1H- |
| | pyrazole-1-carboxamide |
| 2170 | 5-cyclobutyl-N-(4-fluorobenzyl)-3-[5-(2-furoylamino)-2-hydroxyphenyl]-1H- |
| | pyrazole-1-carboxamide |
| 2171 | 5-cyclobutyl-N-(3,4-dichlorobenzyl)-3-[5-(2-furoylamino)-2-hydroxyphenyl]-1H- |
| | pyrazole-1-carboxamide |
| 2172 | 5-cyclobutyl-3-[5-(2-furoylamino)-2-hydroxyphenyl]-N-(4-methoxybenzyl)-1H- |
| | pyrazole-1-carboxamide |
| 2173 | N-(sec-butyl)-5-cyclobutyl-3-[5-(2-furoylamino)-2-hydroxyphenyl]-1H-pyrazole- |
| | 1-carboxamide |
| 2174 | 5-cyclobutyl-N-(2-fluorobenzyl)-3-[5-(2-furoylamino)-2-hydroxyphenyl]-1H- |
| | pyrazole-1-carboxamide |
| 2175 | 5-cyclobutyl-N-(3-fluorobenzyl)-3-[5-(2-furoylamino)-2-hydroxyphenyl]-1H- |
| | pyrazole-1-carboxamide |
| 2176 | 5-cyclobutyl-N-cyclopropyl-3-[5-(2-furoylamino)-2-hydroxyphenyl]-1H- |
| | pyrazole-1-carboxamide |
| 2177 | 5-cyclopentyl-3-[5-(2-furoylamino)-2-hydroxyphenyl]-N-isopropyl-1H-pyrazole- |
| | 1-carboxamide |
| 2178 | 5-cyclopentyl-N-ethyl-3-[5-(2-furoylamino)-2-hydroxyphenyl]-1H-pyrazole-1- |
| | carboxamide |
| 2179 | N-allyl-5-cyclopentyl-3-[5-(2-furoylamino)-2-hydroxyphenyl]-1H-pyrazole-1- |
| | carboxamide |
| 2180 | 5-cyclopentyl-3-[5-(2-furoylamino)-2-hydroxyphenyl]-N-propyl-1H-pyrazole-1- |
| | carboxamide |
| 2181 | N-benzyl-5-cyclopentyl-3-[5-(2-furoylamino)-2-hydroxyphenyl]-1H-pyrazole-1- |
| | carboxamide |
| 2182 | 5-cyclopentyl-3-[5-(2-furoylamino)-2-hydroxyphenyl]-N-(2-phenylethyl)-1H- |
| | pyrazole-1-carboxamide |
| 2183 | 5-cyclopentyl-3-[5-(2-furoylamino)-2-hydroxyphenyl]-N-(2-methylbenzyl)-1H- |
| | pyrazole-1-carboxamide |
| 2184 | 5-cyclopentyl-3-[5-(2-furoylamino)-2-hydroxyphenyl]-N-(3-methylbenzyl)-1H- |
| | pyrazole-1-carboxamide |
| 2185 | 5-cyclopentyl-3-[5-(2-furoylamino)-2-hydroxyphenyl]-N-(4-methylbenzyl)-1H- |
| | pyrazole-1-carboxamide |
| 2186 | N-(2-chlorobenzyl)-5-cyclopentyl-3-[5-(2-furoylamino)-2-hydroxyphenyl]-1H- |
| | pyrazole-1-carboxamide |
| 2187 | 5-cyclopentyl-N-(4-fluorobenzyl)-3-[5-(2-furoylamino)-2-hydroxyphenyl]-1H- |
| | pyrazole-1-carboxamide |
| 2188 | 5-cyclopentyl-N-(3,4-dichlorobenzyl)-3-[5-(2-furoylamino)-2-hydroxyphenyl]- |
| | 1H-pyrazole-1-carboxamide |
| 2189 | 5-cyclopentyl-3-[5-(2-furoylamino)-2-hydroxyphenyl]-N-(4-methoxybenzyl)-1H- |
| | pyrazole-1-carboxamide |
| 2190 | N-(sec-butyl)-5-cyclopentyl-3-[5-(2-furoylamino)-2-hydroxyphenyl]-1H- |
| | pyrazole-1-carboxamide |
| 2191 | 5-cyclopentyl-N-(2-fluorobenzyl)-3-[5-(2-furoylamino)-2-hydroxyphenyl]-1H- |
| | pyrazole-1-carboxamide |
| 2192 | 5-cyclopentyl-N-(3-fluorobenzyl)-3-[5-(2-furoylamino)-2-hydroxyphenyl]-1H- |
| | pyrazole-1-carboxamide |
| 2193 | 5-cyclopentyl-N-cyclopropyl-3-[5-(2-furoylamino)-2-hydroxyphenyl]-1H- |
| | pyrazole-1-carboxamide |
| 2194 | 5-cyclopropyl-3-{2-hydroxy-5-[(thien-2-ylcarbonyl)amino]phenyl}-N-isopropyl- |
| | 1H-pyrazole-1-carboxamide |
| 2195 | 5-cyclopropyl-N-ethyl-3-{2-hydroxy-5-[(thien-2-ylcarbonyl)amino]phenyl}-1H- |
| | pyrazole-1-carboxamide |
| 2196 | N-allyl-5-cyclopropyl-3-{2-hydroxy-5-[(thien-2-ylcarbonyl)amino]phenyl}-1H- |
| | pyrazole-1-carboxamide |
| 2197 | 5-cyclopropyl-3-{2-hydroxy-5-[(thien-2-ylcarbonyl)amino]phenyl}-N-propyl-1H- |
| | pyrazole-1-carboxamide |
| 2198 | N-benzyl-5-cyclopropyl-3-{2-hydroxy-5-[(thien-2-ylcarbonyl)amino]phenyl}-1H- |
| | pyrazole-1-carboxamide |
| 2199 | 5-cyclopropyl-3-{2-hydroxy-5-[(thien-2-ylcarbonyl)amino]phenyl}-N-(2- |
| | phenylethyl)-1H-pyrazole-1-carboxamide |
| 2200 | 5-cyclopropyl-3-{2-hydroxy-5-[(thien-2-ylcarbonyl)amino]phenyl}-N-(2- |
| | methylbenzyl)-1H-pyrazole-1-carboxamide |
| 2201 | 5-cyclopropyl-3-{2-hydroxy-5-[(thien-2-ylcarbonyl)amino]phenyl}-N-(3- |
| | methylbenzyl-1H-pyrazole-1-carboxamide |
| 2202 | 5-cyclopropyl-3-{2-hydroxy-5-[(thien-2-ylcarbonyl)amino]phenyl}-N-(4- |
| | methylbenzyl)-1H-pyrazole-1-carboxamide |
| 2203 | N-(2-chlorobenzyl)-5-cyclopropyl-3-{2-hydroxy-5-[(thien-2- |
| | ylcarbonyl)amino]phenyl}-1H-pyrazole-1-carboxamide |
| 2204 | 5-cyclopropyl-N-(4-fluorobenzyl)-3-{2-hydroxy-5-[(thien-2- |
| | ylcarbonyl)amino]phenyl}-1H-pyrazole-1-carboxamide |
| 2205 | 5-cyclopropyl-N-(3,4-dichlorobenzyl)-3-{2-hydroxy-5-[(thien-2- |
| | ylcarbonyl)amino]phenyl}-1H-pyrazole-1-carboxamide |
| 2206 | 5-cyclopropyl-3-{2-hydroxy-5-[(thien-2-ylcarbonyl)amino]phenyl}-N-(4- |
| | methoxybenzyl)-1H-pyrazole-1-carboxamide |
| 2207 | N-(sec-butyl)-5-cyclopropyl-3-{2-hydroxy-5-[(thien-2- |
| | ylcarbonyl)amino]phenyl}-1H-pyrazole-1-carboxamide |
| 2208 | 5-cyclopropyl-N-(2-fluorobenzyl)-3-{2-hydroxy-5-[(thien-2- |
| | ylcarbonyl)amino]phenyl}-1H-pyrazole-1-carboxamide |
| 2209 | 5-cyclopropyl-N-(3-fluorobenzyl)-3-{2-hydroxy-5-[(thien-2- |
| | ylcarbonyl)amino]phenyl}-1H-pyrazole-1-carboxamide |
| 2210 | N,5-dicyclopropyl-3-{2-hydroxy-5-[(thien-2-ylcarbonyl)amino]phenyl}-1H- |
| | pyrazole-1-carboxamide |
| 2211 | 5-cyclobutyl-3-{2-hydroxy-5-[(thien-2-ylcarbonyl)amino]phenyl}-N-isopropyl- |
| | 1H-pyrazole-1-carboxamide |
| 2212 | 5-cyclobutyl-N-ethyl-3-{2-hydroxy-5-[(thien-2-ylcarbonyl)amino]phenyl}-1H- |
| | pyrazole-1-carboxamide |
| 2213 | N-allyl-5-cyclobutyl-3-{2-hydroxy-5-[(thien-2-ylcarbonyl)amino]phenyl}-1H- |
| | pyrazole-1-carboxamide |
| 2214 | 5-cyclobutyl-3-{2-hydroxy-5-[(thien-2-ylcarbonyl)amino]phenyl}-N-propyl-1H- |
| | pyrazole-1-carboxamide |
| 2215 | N-benzyl-5-cyclobutyl-3-{2-hydroxy-5-[(thien-2-ylcarbonyl)amino]phenyl}-1H- |
| | pyrazole-1-carboxamide |
| 2216 | 5-cyclobutyl-3-{2-hydroxy-5-[(thien-2-ylcarbonyl)amino]phenyl}-N-(2- |
| | phenylethyl)-1H-pyrazole-1-carboxamide |
| 2217 | 5-cyclobutyl-3-{2-hydroxy-5-[(thien-2-ylcarbonyl)amino]phenyl}-N-(2- |
| | methylbenzyl)-1H-pyrazole-1-carboxamide |
| 2218 | 5-cyclobutyl-3-{2-hydroxy-5-[(thien-2-ylcarbonyl)amino]phenyl}-N-(3- |
| | methylbenzyl)-1H-pyrazole-1-carboxamide |
| 2219 | 5-cyclobutyl-3-{2-hydroxy-5-[(thien-2-ylcarbonyl)amino]phenyl}-N-(4- |
| | methylbenzyl)-1H-pyrazole-1-carboxamide |
| 2220 | N-(2-chlorobenzyl)-5-cyclobutyl-3-{2-hydroxy-5-[(thien-2- |
| | ylcarbonyl)amino]phenyl}-1H-pyrazole-1-carboxamide |
| 2221 | 5-cyclobutyl-N-(4-fluorobenzyl)-3-{2-hydroxy-5-[(thien-2- |
| | ylcarbonyl)amino]phenyl}-1H-pyrazole-1-carboxamide |
| 2222 | 5-cyclobutyl-N-(3,4-dichlorobenzyl)-3-{2-hydroxy-5 -[(thien-2- |
| | ylcarbonyl)amino]phenyl}-1H-pyrazole-1-carboxamide |
| 2223 | 5-cyclobutyl-3-{2-hydroxy-5-[(thien-2-ylcarbonyl)amino]phenyl}-N-(4- |
| | methoxybenzyl)-1H-pyrazole-1-carboxamide |
| 2224 | N-(sec-butyl)-5-cyclobutyl-3-{2-hydroxy-5-[(thien-2-ylcarbonyl)amino]phenyl}- |
| | 1H-pyrazole-1-carboxamide |
| 2225 | 5-cyclobutyl-N-(2-fluorobenzyl)-3-{2-hydroxy-5-[(thien-2- |
| | ylcarbonyl)amino]phenyl}-1H-pyrazole-1-carboxamide |
| 2226 | 5-cyclobutyl-N-(3-fluorobenzyl)-3-{2-hydroxy-5-[(thien-2- |
| | ylcarbonyl)amino]phenyl}-1H-pyrazole-1-carboxamide |
| 2227 | 5-cyclobutyl-N-cyclopropyl-3-{2-hydroxy-5-[(thien-2-ylcarbonyl)amino]phenyl}- |
| | 1H-pyrazole-1-carboxamide |
| 2228 | 5-cyclopentyl-3-{2-hydroxy-5-[(thien-2-ylcarbonyl)amino]phenyl}-N-isopropyl- |
| | 1H-pyrazole-1-carboxamide |
| 2229 | 5-cyclopentyl-N-ethyl-3-{2-hydroxy-5-[(thien-2-ylcarbonyl)amino]phenyl}-1H- |
| | pyrazole-1-carboxamide |
| 2230 | N-allyl-5-cyclopentyl-3-{2-hydroxy-5-[(thien-2-ylcarbonyl)amino]phenyl}-1H- |
| | pyrazole-1-carboxamide |
| 2231 | 5-cyclopentyl-3-{2-hydroxy-5-[(thien-2-ylcarbonyl)amino]phenyl}-N-propyl-1H- |
| | pyrazole-1-carboxamide |
| 2232 | N-benzyl-5-cyclopentyl-3-{2-hydroxy-5-[(thien-2-ylcarbonyl)amino]phenyl}-1H- |
| | pyrazole-1-carboxamide |
| 2233 | 5-cyclopentyl-3-{2-hydroxy-5-[(thien-2-ylcarbonyl)amino]phenyl}-N-(2- |
| | phenylethyl)-1H-pyrazole-1-carboxamide |
| 2234 | 5-cyclopentyl-3-{2-hydroxy-5-[(thien-2-ylcarbonyl)amino]phenyl}-N-(2- |
| | methylbenzyl)-1H-pyrazole-1-carboxamide |
| 2235 | 5-cyclopentyl-3-{2-hydroxy-5-[(thien-2-ylcarbonyl)amino]phenyl}-N-(3- |
| | methylbenzyl)-1H-pyrazole-1-carboxamide |
| 2236 | 5-cyclopentyl-3-{2-hydroxy-5-[(thien-2-ylcarbonyl)amino]phenyl}-N-(4- |
| | methylbenzyl)-1H-pyrazole-1-carboxamide |
| 2237 | N-(2-chlorobenzyl)-5-cyclopentyl-3-{2-hydroxy-5-[(thien-2- |
| | ylcarbonyl)amino]phenyl}-1H-pyrazole-1-carboxamide |
| 2238 | 5-cyclopentyl-N-(4-fluorobenzyl)-3-{2-hydroxy-5-[(thien-2- |
| | ylcarbonyl)amino]phenyl}-1H-pyrazole-1-carboxamide |
| 2239 | 5-cyclopentyl-N-(3,4-dichlorobenzyl)-3-{2-hydroxy-5-(thien-2- |
| | ylcarbonyl)amino]phenyl}-1H-pyrazole-1-carboxamide |
| 2240 | 5-cyclopentyl-3-{2-hydroxy-5-[(thien-2-ylcarbonyl)amino]phenyl}-N-(4- |
| | methoxybenzyl)-1H-pyrazole-1-carboxamide |
| 2241 | N-(sec-butyl)-5-cyclopentyl-3-{2-hydroxy-5-[(thien-2-ylcarbonyl)amino]phenyl}- |
| | 1H-pyrazole-1-carboxamide |
| 2242 | 5-cyclopentyl-N-(2-fluorobenzyl)-3-{2-hydroxy-5-[(thien-2- |
| | ylcarbonyl)amino]phenyl}-1H-pyrazole-1-carboxamide |
| 2243 | 5-cyclopentyl-N-(3-fluorobenzyl)-3-{2-hydroxy-5-[(thien-2- |
| | ylcarbonyl)amino]phenyl}-1H-pyrazole-1-carboxamide |
| 2244 | 5-cyclopentyl-N-cyclopropyl-3-{2-hydroxy-5-[(thien-2- |
| | ylcarbonyl)amino]phenyl}-1H-pyrazole-1-carboxamide |
| 2245 | N-(3-{5-cyclopropyl-1-[(isopropylamino)carbonyl]-1H-pyrazol-3-yl}-4- |
| | hydroxyphenyl)quinoxaline-2-carboxamide |
| 2246 | N-(3-{5-cyclopropyl-1-[(ethylamino)carbonyl]-1H-pyrazol-3-yl}-4- |
| | hydroxyphenyl)quinoxaline-2-carboxamide |
| 2247 | N-(3-{1-[(allylamino)carbonyl]-5-cyclopropyl-1H-pyrazol-3-yl}-4- |
| | hydroxyphenyl)quinoxaline-2-carboxamide |
| 2248 | N-(3-{5-cyclopropyl-1-[(propylamino)carbonyl]-1H-pyrazol-3-yl}-4- |
| | hydroxyphenyl)quinoxaline-2-carboxamide |
| 2249 | N-(3-{1-[(benzylamino)carbonyl]-5-cyclopropyl-1H-pyrazol-3-yl}-4- |
| | hydroxyphenyl)quinoxaline-2-carboxamide |
| 2250 | N-[3-(5-cyclopropyl-1-{[(2-phenylethyl)amino]carbonyl}-1H-pyrazol-3-yl)-4- |
| | hydroxyphenyl]quinoxaline-2-carboxamide |
| 2251 | N-[3-(5-cyclopropyl-1-{[(2-methylbenzyl)amino]carbonyl}-1H-pyrazol-3-yl)-4- |
| | hydroxyphenyl]quinoxaline-2-carboxamide |
| 2252 | N-[3-(5-cyclopropyl-1-{[(3-methylbenzyl)amino]carbonyl}-1H-pyrazol-3-yl)-4- |
| | hydroxyphenyl]quinoxaline-2-carboxamide |
| 2253 | N-[3-(5-cyclopropyl-1-{[(4-methylbenzyl)amino]carbonyl}-1H-pyrazol-3-yl)-4- |
| | hydroxyphenyl]quinoxaline-2-carboxamide |
| 2254 | N-[3-(1-{[(2-chlorobenzyl)amino]carbonyl}-5-cyclopropyl-1H-pyrazol-3-yl)-4- |
| | hydroxyphenyl]quinoxaline-2-carboxamide |
| 2255 | N-[3-(5-cyclopropyl-1-{[(4-fluorobenzyl)amino]carbonyl}-1H-pyrazol-3-yl)-4- |
| | hydroxyphenyl]quinoxaline-2-carboxamide |
| 2256 | N-[3-(5-cyclopropyl-1-{[(3,4-dichlorobenzyl)amino]carbonyl}-1H-pyrazol-3-yl)- |
| | 4-hydroxyphenyl]quinoxaline-2-carboxamide |
| 2257 | N-[3-(5-cyclopropyl-1-{[(4-methoxybenzyl)amino]carbonyl}-1H-pyrazol-3-yl)-4- |
| | hydroxyphenyl]quinoxaline-2-carboxamide |
| 2258 | N-(3-{1-[(sec-butylamino)carbonyl]-5-cyclopropyl-1H-pyrazol-3-yl}-4- |
| | hydroxyphenyl)quinoxaline-2-carboxamide |
| 2259 | N-[3-(5-cyclopropyl-1-{[(2-fluorobenzyl)amino]carbonyl}-1H-pyrazol-3-yl)-4- |
| | hydroxyphenyl]quinoxaline-2-carboxamide |
| 2260 | N-[3-(5-cyclopropyl-1-{[(3-fluorobenzyl)amino]carbonyl}-1H-pyrazol-3-yl)-4- |
| | hydroxyphenyl]quinoxaline-2-carboxamide |
| 2261 | N-(3-{5-cyclopropyl-1-[(cyclopropylamino)carbonyl]-1H-pyrazol-3-yl}-4- |
| | hydroxyphenyl)quinoxaline-2-carboxamide |
| 2262 | N-(3-{5-cyclobutyl-1-[(isopropylamino)carbonyl]-1H-pyrazol-3-yl}-4- |
| | hydroxyphenyl)quinoxaline-2-carboxamide |
| 2263 | N-(3-{5-cyclobutyl-1-[(ethylamino)carbonyl]-1H-pyrazol-3-yl}-4- |
| | hydroxyphenyl)quinoxaline-2-carboxamide |
| 2264 | N-(3-{1-[(allylamino)carbonyl]-5-cyclobutyl-1H-pyrazol-3-yl}-4- |
| | hydroxyphenyl)quinoxaline-2-carboxamide |
| 2265 | N-(3-{5-cyclobutyl-1-[(propylamino)carbonyl]-1H-pyrazol-3-yl}-4- |
| | hydroxyphenyl)quinoxaline-2-carboxamide |
| 2266 | N-(3-{1-[(benzylamino)carbonyl]-5-cyclobutyl-1H-pyrazol-3-yl}-4- |
| | hydroxyphenyl)quinoxaline-2-carboxamide |
| 2267 | N-[3-(5-cyclobutyl-1-{[(2-phenylethyl)amino]carbonyl}-1H-pyrazol-3-yl)-4- |
| | hydroxyphenyl]quinoxaline-2-carboxamide |
| 2268 | N-[3-(5-cyclobutyl-1-{[(2-methylbenzyl)amino]carbonyl}-1H-pyrazol-3-yl)-4- |
| | hydroxyphenyl]quinoxaline-2-carboxamide |
| 2269 | N-[3-(5-cyclobutyl-1-{[(3-methylbenzyl)amino]carbonyl}-1H-pyrazol-3-yl)-4- |
| | hydroxyphenyl]quinoxaline-2-carboxamide |
| 2270 | N-[3-(5-cyclobutyl-1-{[(4-methylbenzyl)amino]carbonyl}-1H-pyrazol-3-yl)-4- |
| | hydroxyphenyl]quinoxaline-2-carboxamide |
| 2271 | N-[3-(1-{[(2-chlorobenzyl)amino]carbonyl}-5-cyclobutyl-1H-pyrazol-3-yl)-4- |
| | hydroxyphenyl]quinoxaline-2-carboxamide |
| 2272 | N-[3-(5-cyclobutyl-1-{[(4-fluorobenzyl)amino]carbonyl}-1H-pyrazol-3-yl)-4- |
| | hydroxyphenyl]quinoxaline-2-carboxamide |
| 2273 | N-[3-(5-cyclobutyl-1-{[(3,4-dichlorobenzyl)amino]carbonyl}-1H-pyrazol-3-yl)-4- |
| | hydroxyphenyl]quinoxaline-2-carboxamide |
| 2274 | N-[3-(5-cyclobutyl-1-{[(4-methoxybenzyl)amino]carbonyl}-1H-pyrazol-3-yl)-4- |
| | hydroxyphenyl]quinoxaline-2-carboxamide |
| 2275 | N-(3-{1-[(sec-butylamino)carbonyl]-5-cyclobutyl-1H-pyrazol-3-yl}-4- |
| | hydroxyphenyl)quinoxaline-2-carboxamide |
| 2276 | N-[3-(5-cyclobutyl-1-{[(2-fluorobenzyl)amino]carbonyl}-1H-pyrazol-3-yl)-4- |
| | hydroxyphenyl]quinoxaline-2-carboxamide |
| 2277 | N-[3-(5-cyclobutyl-1-{[(3-fluorobenzyl)amino]carbonyl}-1H-pyrazol-3-yl)-4- |
| | hydroxyphenyl]quinoxaline-2-carboxamide |
| 2278 | N-(3-{5-cyclobutyl-1-[(cyclopropylamino)carbonyl]-1H-pyrazol-3-yl}-4- |
| | hydroxyphenyl)quinoxaline-2-carboxamide |
| 2279 | N-(3-{5-cyclopentyl-1-[(isopropylamino)carbonyl]-1H-pyrazol-3-yl}-4- |
| | hydroxyphenyl)quinoxaline-2-carboxamide |
| 2280 | N-(3-{5-cyclopentyl-1-[(ethylamino)carbonyl]-1H-pyrazol-3-yl}-4- |
| | hydroxyphenyl)quinoxaline-2-carboxamide |
| 2281 | N-(3-{1-[(allylamino)carbonyl]-5-cyclopentyl-1H-pyrazol-3-yl}-4- |
| | hydroxyphenyl)quinoxaline-2-carboxamide |
| 2282 | N-(3-{5-cyclopentyl-1-[(propylamino)carbonyl]-1H-pyrazol-3-yl}-4- |
| | hydroxyphenyl)quinoxaline-2-carboxamide |
| 2283 | N-(3-{1-[(benzylamino)carbonyl]-5-cyclopentyl-1H-pyrazol-3-yl}-4- |
| | hydroxyphenyl)quinoxaline-2-carboxamide |
| 2284 | N-[3-(5-cyclopentyl-1-{[(2-phenylethyl)amino]carbonyl}-1H-pyrazol-3-yl)-4- |
| | hydroxyphenyl]quinoxaline-2-carboxamide |
| 2285 | N-[3-(5-cyclopentyl-1-{[(2-methylbenzyl)amino]carbonyl}-1H-pyrazol-3-yl)-4- |
| | hydroxyphenyl]quinoxaline-2-carboxamide |
| 2286 | N-[3-(5-cyclopentyl-1-{[(3-methylbenzyl)amino]carbonyl}-1H-pyrazol-3-yl)-4- |
| | hydroxyphenyl]quinoxaline-2-carboxamide |
| 2287 | N-[3-(5-cyclopentyl-1-{[(4-methylbenzyl)amino]carbonyl}-1H-pyrazol-3-yl)-4- |
| | hydroxyphenyl]quinoxaline-2-carboxamide |
| 2288 | N-[3-(1-{[(2-chlorobenzyl)amino]carbonyl}-5-cyclopentyl-1H-pyrazol-3-yl)-4- |
| | hydroxyphenyl]quinoxaline-2-carboxamide |
| 2289 | N-[3-(5-cyclopentyl-1-{[(4-fluorobenzyl)amino]carbonyl}-1H-pyrazol-3-yl)-4- |
| | hydroxyphenyl]quinoxaline-2-carboxamide |
| 2290 | N-[3-(5-cyclopentyl-1-{[(3,4-dichlorobenzyl)amino]carbonyl}-1H-pyrazol-3-yl)- |
| | 4-hydroxyphenyl]quinoxaline-2-carboxamide |
| 2291 | N-[3-(5-cyclopentyl-1-{[(4-methoxybenzyl)amino]carbonyl}-1H-pyrazol-3-yl)-4- |
| | hydroxyphenyl]quinoxaline-2-carboxamide |
| 2292 | N-(3-{1-[(sec-butylamino)carbonyl]-5-cyclopentyl-1H-pyrazol-3-yl}-4- |
| | hydroxyphenyl)quinoxaline-2-carboxamide |
| 2293 | N-[3-(5-cyclopentyl-1-{[(2-fluorobenzyl)amino]carbonyl}-1H-pyrazol-3-yl)-4- |
| | hydroxyphenyl]quinoxaline-2-carboxamide |
| 2294 | N-[3-(5-cyclopentyl-1-{[(3-fluorobenzyl)amino]carbonyl}-1H-pyrazol-3-yl)-4- |
| | hydroxyphenyl]quinoxaline-2-carboxamide |
| 2295 | N-(3-{5-cyclopentyl-1-[(cyclopropylamino)carbonyl]-1H-pyrazol-3-yl}-4- |
| | hydroxyphenyl)quinoxaline-2-carboxamide |
| 2296 | N-(3-{5-cyclopropyl-1-[(isopropylamino)carbonyl]-1H-pyrazol-3-yl}-4- |
| | hydroxyphenyl)nicotinamide |
| 2297 | N-(3-{5-cyclopropyl-1-[(ethylamino)carbonyl]-1H-pyrazol-3-yl}-4- |
| | hydroxyphenyl)nicotinamide |
| 2298 | N-(3-{1-[(allylamino)carbonyl]-5-cyclopropyl-1H-pyrazol-3-yl}-4- |
| | hydroxyphenyl)nicotinamide |
| 2299 | N-(3-{5-cyclopropyl-1-[(propylamino)carbonyl]-1H-pyrazol-3-yl}-4- |
| | hydroxyphenyl)nicotinamide |
| 2300 | N-(3-{1-[(benzylamino)carbonyl]-5-cyclopropyl-1H-pyrazol-3-yl}-4- |
| | hydroxyphenyl)nicotinamide |
| 2301 | N-[3-(5-cyclopropyl-1-{[(2-phenylethyl)amino]carbonyl}-1H-pyrazol-3-yl)-4- |
| | hydroxyphenyl]nicotinamide |
| 2302 | N-[3-(5-cyclopropyl-1-{[(2-methylbenzyl)amino]carbonyl}-1H-pyrazol-3-yl)-4- |
| | hydroxyphenyl]nicotinamide |
| 2303 | N-[3-(5-cyclopropyl-1-{[(3-methylbenzyl)amino]carbonyl}-1H-pyrazol-3-yl)-4- |
| | hydroxyphenyl]nicotinamide |
| 2304 | N-[3-(5-cyclopropyl-1-{[(4-methylbenzyl)amino]carbonyl}-1H-pyrazol-3-yl)-4- |
| | hydroxyphenyl]nicotinamide |
| 2305 | N-[3-(1-{[(2-chlorobenzyl)amino]carbonyl}-5-cyclopropyl-1H-pyrazol-3-yl)-4- |
| | hydroxyphenyl]nicotinamide |
| 2306 | N-[3-(5-cyclopropyl-1-{[(4-fluorobenzyl)amino]carbonyl}-1H-pyrazol-3-yl)-4- |
| | hydroxyphenyl]nicotinamide |
| 2307 | N-[3-(5-cyclopropyl-1-{[(3,4-dichlorobenzyl)amino]carbonyl}-1H-pyrazol-3-yl)- |
| | 4-hydroxyphenyl]nicotinamide |
| 2308 | N-[3-(5-cyclopropyl-1-{[(4-methoxybenzyl)amino]carbonyl}-1H-pyrazol-3-yl)-4- |
| | hydroxyphenyl]nicotinamide |
| 2309 | N-(3-{1-[(sec-butylamino)carbonyl]-5-cyclopropyl-1H-pyrazol-3-yl}-4- |
| | hydroxyphenyl)nicotinamide |
| 2310 | N-[3-(5-cyclopropyl-1-{[(2-fluorobenzyl)amino]carbonyl}-1H-pyrazol-3-yl)-4- |
| | hydroxyphenyl]nicotinamide |
| 2311 | N-[3-(5-cyclopropyl-1-{[(3-fluorobenzyl)amino]carbonyl}-1H-pyrazol-3-yl)-4- |
| | hydroxyphenyl]nicotinamide |
| 2312 | N-(3-{5-cyclopropyl-1-[(cyclopropylamino)carbonyl]-1H-pyrazol-3-yl}-4- |
| | hydroxyphenyl)nicotinamide |
| 2313 | N-(3-{5-cyclobutyl-1-[(isopropylamino)carbonyl]-1H-pyrazol-3-yl}-4- |
| | hydroxyphenyl)nicotinamide |
| 2314 | N-(3-{5-cyclobutyl-1-[(ethylamino)carbonyl]-1H-pyrazol-3-yl}-4- |
| | hydroxyphenyl)nicotinamide |
| 2315 | N-(3-{1-[(allylamino)carbonyl]-5-cyclobutyl-1H-pyrazol-3-yl}-4- |
| | hydroxyphenyl)nicotinamide |
| 2316 | N-(3-{5-cyclobutyl-1-[(propylamino)carbonyl]-1H-pyrazol-3-yl}-4- |
| | hydroxyphenyl)nicotinamide |
| 2317 | N-(3-{1-[(benzylamino)carbonyl]-5-cyclobutyl-1H-pyrazol-3-yl}-4- |
| | hydroxyphenyl)nicotinamide |
| 2318 | N-[3-(5-cyclobutyl-1-{[(2-phenylethyl)amino]carbonyl}-1H-pyrazol-3-yl)-4- |
| | hydroxyphenyl]nicotinamide |
| 2319 | N-[3-(5-cyclobutyl-1-{[(2-methylbenzyl)amino]carbonyl}-1H-pyrazol-3-yl)-4- |
| | hydroxyphenyl]nicotinamide |
| 2320 | N-[3-(5-cyclobutyl-1-{[(3-methylbenzyl)amino]carbonyl}-1H-pyrazol-3-yl)-4- |
| | hydroxyphenyl]nicotinamide |
| 2321 | N-[3-(5-cyclobutyl-1-{[(4-methylbenzyl)amino]carbonyl}-1H-pyrazol-3-yl)-4- |
| | hydroxyphenyl]nicotinamide |
| 2322 | N-[3-(1-{[(2-chlorobenzyl)amino]carbonyl}-5-cyclobutyl-1H-pyrazol-3-yl)-4- |
| | hydroxyphenyl]nicotinamide |
| 2323 | N-[3-(5-cyclobutyl-1-{[(4-fluorobenzyl)amino]carbonyl}-1H-pyrazol-3-yl)-4- |
| | hydroxyphenyl]nicotinamide |
| 2324 | N-[3-(5-cyclobutyl-1-{[(3,4-dichlorobenzyl)amino]carbonyl}-1H-pyrazol-3-yl)-4- |
| | hydroxyphenyl]nicotinamide |
| 2325 | N-[3-(5-cyclobutyl-1-{[(4-methoxybenzyl)amino]carbonyl}-1H-pyrazol-3-yl)-4- |
| | hydroxyphenyl]nicotinamide |
| 2326 | N-(3-{1-[(sec-butylamino)carbonyl]-5-cyclobutyl-1H-pyrazol-3-yl}-4- |
| | hydroxyphenyl)nicotinamide |
| 2327 | N-[3-(5-cyclobutyl-1-{[(2-fluorobenzyl)amino]carbonyl}-1H-pyrazol-3-yl)-4- |
| | hydroxyphenyl]nicotinamide |
| 2328 | N-[3-(5-cyclobutyl-1-{[(3-fluorobenzyl)amino]carbonyl}-1H-pyrazol-3-yl)-4- |
| | hydroxyphenyl]nicotinamide |
| 2329 | N-(3-{5-cyclobutyl-1-[(cyclopropylamino)carbonyl]-1H-pyrazol-3-yl}-4- |
| | hydroxyphenyl)nicotinamide |
| 2330 | N-(3-{5-cyclopentyl-1-[(isopropylamino)carbonyl]-1H-pyrazol-3-yl}-4- |
| | hydroxyphenyl)nicotinamide |
| 2331 | N-(3-{5-cyclopentyl-1-[(ethylamino)carbonyl]-1H-pyrazol-3-yl}-4- |
| | hydroxyphenyl)nicotinamide |
| 2332 | N-(3-{1-[(allylamino)carbonyl]-5-cyclopentyl-1H-pyrazol-3-yl}-4- |
| | hydroxyphenyl)nicotinamide |
| 2333 | N-(3-{5-cyclopentyl-1-[(propylamino)carbonyl]-1H-pyrazol-3-yl}-4- |
| | hydroxyphenyl)nicotinamide |
| 2334 | N-(3-{1-[(benzylamino)carbonyl]-5-cyclopentyl-1H-pyrazol-3-yl}-4- |
| | hydroxyphenyl)nicotinamide |
| 2335 | N-[3-(5-cyclopentyl-1-{[(2-phenylethyl)amino]carbonyl}-1H-pyrazol-3-yl)-4- |
| | hydroxyphenyl]nicotinamide |
| 2336 | N-[3-(5-cyclopentyl-1-{[(2-methylbenzyl)amino]carbonyl}-1H-pyrazol-3-yl)-4- |
| | hydroxyphenyl]nicotinamide |
| 2337 | N-[3-(5-cyclopentyl-1-{[(3-methylbenzyl)amino]carbonyl}-1H-pyrazol-3-yl)-4- |
| | hydroxyphenyl]nicotinamide |
| 2338 | N-[3-(5-cyclopentyl-1-{[(4-methylbenzyl)amino]carbonyl}-1H-pyrazol-3-yl)-4- |
| | hydroxyphenyl]nicotinamide |
| 2339 | N-[3-(1-{[(2-chlorobenzyl)amino]carbonyl}-5-cyclopentyl-1H-pyrazol-3-yl)-4- |
| | hydroxyphenyl]nicotinamide |
| 2340 | N-[3-(5-cyclopentyl-1-{[(4-fluorobenzyl)amino]carbonyl}-1H-pyrazol-3-yl)-4- |
| | hydroxyphenyl]nicotinamide |
| 2341 | N-[3-(5-cyclopentyl-1-{[(3,4-dichlorobenzyl)amino]carbonyl}-1H-pyrazol-3-yl)- |
| | 4-hydroxyphenyl]nicotinamide |
| 2342 | N-[3-(5-cyclopentyl-1-{[(4-methoxybenzyl)amino]carbonyl}-1H-pyrazol-3-yl)-4- |
| | hydroxyphenyl]nicotinamide |
| 2343 | N-(3-{1-[(sec-butylamino)carbonyl]-5-cyclopentyl-1H-pyrazol-3-yl}-4- |
| | hydroxyphenyl)nicotinamide |
| 2344 | N-[3-(5-cyclopentyl-1-{[(2-fluorobenzyl)amino]carbonyl}-1H-pyrazol-3-yl)-4- |
| | hydroxyphenyl]nicotinamide |
| 2345 | N-[3-(5-cyclopentyl-1-{[(3-fluorobenzyl)amino]carbonyl}-1H-pyrazol-3-yl)-4- |
| | hydroxyphenyl]nicotinamide |
| 2346 | N-(3-{5-cyclopentyl-1-[(cyclopropylamino)carbonyl]-1H-pyrazol-3-yl}-4- |
| | hydroxyphenyl)nicotinamide |
| 2347 | N-(3-{5-cyclopropyl-1-[(isopropylamino)carbonyl]-1H-pyrazol-3-yl}-4- |
| | hydroxyphenyl)isonicotinamide |
| 2348 | N-(3-{5-cyclopropyl-1-[(ethylamino)carbonyl]-1H-pyrazol-3-yl}-4- |
| | hydroxyphenyl)isonicotinamide |
| 2349 | N-(3-{1-[(allylamino)carbonyl]-5-cyclopropyl-1H-pyrazol-3-yl}-4- |
| | hydroxyphenyl)isonicotinamide |
| 2350 | N-(3-{5-cyclopropyl-1-[(propylamino)carbonyl]-1H-pyrazol-3-yl}-4- |
| | hydroxyphenyl)isonicotinamide |
| 2351 | N-(3-{1-[(benzylamino)carbonyl]-5-cyclopropyl-1H-pyrazol-3-yl}-4- |
| | hydroxyphenyl)isonicotinamide |
| 2352 | N-[3-(5-cyclopropyl-1-{[(2-phenylethyl)amino]carbonyl}-1H-pyrazol-3-yl)-4- |
| | hydroxyphenyl]isonicotinamide |
| 2353 | N-[3-(5-cyclopropyl-1-{[(2-methylbenzyl)amino]carbonyl}-1H-pyrazol-3-yl)-4- |
| | hydroxyphenyl]isonicotinamide |
| 2354 | N-[3-(5-cyclopropyl-1-{[(3-methylbenzyl)amino]carbonyl}-1H-pyrazol-3-yl)-4- |
| | hydroxyphenyl]isonicotinamide |
| 2355 | N-[3-(5-cyclopropyl-1-{[(4-methylbenzyl)amino]carbonyl}-1H-pyrazol-3-yl)-4- |
| | hydroxyphenyl]isonicotinamide |
| 2356 | N-[3-(1-{[(2-chlorobenzyl)amino]carbonyl}-5-cyclopropyl-1H-pyrazol-3-yl)-4- |
| | hydroxyphenyl]isonicotinamide |
| 2357 | N-[3-(5-cyclopropyl-1-{[(4-fluorobenzyl)amino]carbonyl}-1H-pyrazol-3-yl)-4- |
| | hydroxyphenyl]isonicotinamide |
| 2358 | N-[3-(5-cyclopropyl-1-{[(3,4-dichlorobenzyl)amino]carbonyl}-1H-pyrazol-3-yl)- |
| | 4-hydroxyphenyl]isonicotinamide |
| 2359 | N-[3-(5-cyclopropyl-1-{[(4-methoxybenzyl)amino]carbonyl}-1H-pyrazol-3-yl)-4- |
| | hydroxyphenyl]isonicotinamide |
| 2360 | N-(3-{1-[(sec-butylamino)carbonyl]-5-cyclopropyl-1H-pyrazol-3-yl}-4- |
| | hydroxyphenyl)isonicotinamide |
| 2361 | N-[3-(5-cyclopropyl-1-{[(2-fluorobenzyl)amino]carbonyl}-1H-pyrazol-3-yl)-4- |
| | hydroxyphenyl]isonicotinamide |
| 2362 | N-[3-(5-cyclopropyl-1-{[(3 -fluorobenzyl)amino]carbonyl}-1H-pyrazol-3-yl)-4- |
| | hydroxyphenyl]isonicotinamide |
| 2363 | N-(3-{5-cyclopropyl-1-[(cyclopropylamino)carbonyl]-1H-pyrazol-3-yl}-4- |
| | hydroxyphenyl)isonicotinamide |
| 2364 | N-(3-{5-cyclobutyl-1-[(isopropylamino)carbonyl]-1H-pyrazol-3-yl}-4- |
| | hydroxyphenyl)isonicotinamide |
| 2365 | N-(3-{5-cyclobutyl-1-[(ethylamino)carbonyl]-1H-pyrazol-3-yl}-4- |
| | hydroxyphenyl)isonicotinamide |
| 2366 | N-(3-{1-[(allylamino)carbonyl]-5-cyclobutyl-1H-pyrazol-3-yl}-4- |
| | hydroxyphenyl)isonicotinamide |
| 2367 | N-(3-{5-cyclobutyl-1-[(propylamino)carbonyl]-1H-pyrazol-3-yl}-4- |
| | hydroxyphenyl)isonicotinamide |
| 2368 | N-(3-{1-[(benzylamino)carbonyl]-5-cyclobutyl-1H-pyrazol-3-yl}-4- |
| | hydroxyphenyl)isonicotinamide |
| 2369 | N-[3-(5-cyclobutyl-1-{[(2-phenylethyl)amino]carbonyl}-1H-pyrazol-3-yl)-4- |
| | hydroxyphenyl]isonicotinamide |
| 2370 | N-[3-(5-cyclobutyl-1-{[(2-methylbenzyl)amino]carbonyl}-1H-pyrazol-3-yl)-4- |
| | hydroxyphenyl]isonicotinamide |
| 2371 | N-[3-(5-cyclobutyl-1-{[(3-methylbenzyl)amino]carbonyl}-1H-pyrazol-3-yl)-4- |
| | hydroxyphenyl]isonicotinamide |
| 2372 | N-[3-(5-cyclobutyl-1-{[(4-methylbenzyl)amino]carbonyl}-1H-pyrazol-3-yl)-4- |
| | hydroxyphenyl]isonicotinamide |
| 2373 | N-[3-(1-{[(2-chlorobenzyl)amino]carbonyl}-5-cyclobutyl-1H-pyrazol-3-yl)-4- |
| | hydroxyphenyl]isonicotinamide |
| 2374 | N-[3-(5-cyclobutyl-1-{[(4-fluorobenzyl)amino]carbonyl}-1H-pyrazol-3-yl)-4- |
| | hydroxyphenyl]isonicotinamide |
| 2375 | N-[3-(5-cyclobutyl-1-{[(3,4-dichlorobenzyl)amino]carbonyl}-1H-pyrazol-3-yl)-4- |
| | hydroxyphenyl]isonicotinamide |
| 2376 | N-[3-(5-cyclobutyl-1-{[(4-methoxybenzyl)amino]carbonyl}-1H-pyrazol-3-yl)-4- |
| | hydroxyphenyl]isonicotinamide |
| 2377 | N-(3-{1-[(sec-butylamino)carbonyl]-5-cyclobutyl-1H-pyrazol-3-yl}-4- |
| | hydroxyphenyl)isonicotinamide |
| 2378 | N-[3-(5-cyclobutyl-1-{[(2-fluorobenzyl)amino]carbonyl}-1H-pyrazol-3-yl)-4- |
| | hydroxyphenyl]isonicotinamide |
| 2379 | N-[3-(5-cyclobutyl-1-{[(3-fluorobenzyl)amino]carbonyl}-1H-pyrazol-3-yl)-4- |
| | hydroxyphenyl]isonicotinamide |
| 2380 | N-(3-{5-cyclobutyl-1-[(cyclopropylamino)carbonyl]-1H-pyrazol-3-yl}-4- |
| | hydroxyphenyl)isonicotinamide |
| 2381 | N-(3-{5-cyclopentyl-1-[(isopropylamino)carbonyl]-1H-pyrazol-3-yl}-4- |
| | hydroxyphenyl)isonicotinamide |
| 2382 | N-(3-{5-cyclopentyl-1-[(ethylamino)carbonyl]-1H-pyrazol-3-yl}-4- |
| | hydroxyphenyl)isonicotinamide |
| 2383 | N-(3-{1-[(allylamino)carbonyl]-5-cyclopentyl-1H-pyrazol-3-yl}-4- |
| | hydroxyphenyl)isonicotinamide |
| 2384 | N-(3-{5-cyclopentyl-1-[(propylamino)carbonyl]-1H-pyrazol-3-yl}-4- |
| | hydroxyphenyl)isonicotinamide |
| 2385 | N-(3-{1-[(benzylamino)carbonyl]-5-cyclopentyl-1H-pyrazol-3-yl}-4- |
| | hydroxyphenyl)isonicotinamide |
| 2386 | N-[3-(5-cyclopentyl-1-{[(2-phenylethyl)amino]carbonyl}-1H-pyrazol-3-yl)-4- |
| | hydroxyphenyl]isonicotinamide |
| 2387 | N-[3-(5-cyclopentyl-1-{[(2-methylbenzyl)amino]carbonyl}-1H-pyrazol-3-yl)-4- |
| | hydroxyphenyl]isonicotinamide |
| 2388 | N-[3-(5-cyclopentyl-1-{[(3-methylbenzyl)amino]carbonyl}-1H-pyrazol-3-yl)-4- |
| | hydroxyphenyl]isonicotinamide |
| 2389 | N-[3-(5-cyclopentyl-1-{[(4-methylbenzyl)amino]carbonyl}-1H-pyrazol-3-yl)-4- |
| | hydroxyphenyl]isonicotinamide |
| 2390 | N-[3-(1-{[(2-chlorobenzyl)amino]carbonyl}-5-cyclopentyl-1H-pyrazol-3-yl)-4- |
| | hydroxyphenyl]isonicotinamide |
| 2391 | N-[3-(5-cyclopentyl-1-{[(4-fluorobenzyl)amino]carbonyl}-1H-pyrazol-3-yl)-4- |
| | hydroxyphenyl]isonicotinamide |
| 2392 | N-[3-(5-cyclopentyl-1-{[(3,4-dichlorobenzyl)amino]carbonyl}-1H-pyrazol-3-yl)- |
| | 4-hydroxyphenyl]isonicotinamide |
| 2393 | N-[3-(5-cyclopentyl-1-{[(4-methoxybenzyl)amino]carbonyl}-1H-pyrazol-3-yl)-4- |
| | hydroxyphenyl]isonicotinamide |
| 2394 | N-(3-{1-[(sec-butylamino)carbonyl]-5-cyclopentyl-1H-pyrazol-3-yl}-4- |
| | hydroxyphenyl)isonicotinamide |
| 2395 | N-[3-(5-cyclopentyl-1-{[(2-fluorobenzyl)amino]carbonyl}-1H-pyrazol-3-yl)-4- |
| | hydroxyphenyl]isonicotinamide |
| 2396 | N-[3-(5-cyclopentyl-1-{[(3-fluorobenzyl)amino]carbonyl}-1H-pyrazol-3-yl)-4- |
| | hydroxyphenyl]isonicotinamide |
| 2397 | N-(3-{5-cyclopentyl-1-[(cyclopropylamino)carbonyl]-1H-pyrazol-3-yl}-4- |
| | hydroxyphenyl)isonicotinamide |
| 2398 | 3-{5-[(1,3-benzodioxol-5-ylcarbonyl)amino]-2-hydroxyphenyl}-5-cyclopropyl-N- |
| | isopropyl-1H-pyrazole-1-carboxamide |
| 2399 | 3-{5-[(1,3-benzodioxol-5-ylcarbonyl)amino]-2-hydroxyphenyl}-5-cyclopropyl-N- |
| | ethyl-1H-pyrazole-1-carboxamide |
| 2400 | N-allyl-3-{5-[(1,3-benzodioxol-5-ylcarbonyl)amino]-2-hydroxyphenyl}-5- |
| | cyclopropyl-1H-pyrazole-1-carboxamide |
| 2401 | 3-{5-[(1,3-benzodioxol-5-ylcarbonyl)amino]-2-hydroxyphenyl}-5-cyclopropyl-N- |
| | propyl-1H-pyrazole-1-carboxamide |
| 2402 | 3-{5-[(1,3-benzodioxol-5-ylcarbonyl)amino]-2-hydroxyphenyl}-N-benzyl-5- |
| | cyclopropyl-1H-pyrazole-1-carboxamide |
| 2403 | 3-{5-[(1,3-benzodioxol-5-ylcarbonyl)amino]-2-hydroxyphenyl}-5-cyclopropyl-N- |
| | (2-phenylethyl)-1H-pyrazole-1-carboxamide |
| 2404 | 3-{5-[(1,3-benzodioxol-5-ylcarbonyl)amino]-2-hydroxyphenyl}-5-cyclopropyl-N- |
| | (2-methylbenzyl)-1H-pyrazole-1-carboxamide |
| 2405 | 3-{5-[(1,3-benzodioxol-5-ylcarbonyl)amino]-2-hydroxyphenyl}-5-cyclopropyl-N- |
| | (3-methylbenzyl)-1H-pyrazole-1-carboxamide |
| 2406 | 3-{5-[(1,3-benzodioxol-5-ylcarbonyl)amino]-2-hydroxyphenyl}-5-cyclopropyl-N- |
| | (4-methylbenzyl)-1H-pyrazole-1-carboxamide |
| 2407 | 3-{5-[(1,3-benzodioxol-5-ylcarbonyl)amino]-2-hydroxyphenyl}-N-(2- |
| | chlorobenzyl)-5-cyclopropyl-1H-pyrazole-1-carboxamide |
| 2408 | 3-{5-[(1,3-benzodioxol-5-ylcarbonyl)amino]-2-hydroxyphenyl}-5-cyclopropyl-N- |
| | (4-fluorobenzyl)-1H-pyrazole-1-carboxamide |
| 2409 | 3-{5-[(1,3-benzodioxol-5-ylcarbonyl)amino]-2-hydroxyphenyl}-5-cyclopropyl-N- |
| | (3,4-dichlorobenzyl)-1H-pyrazole-1-carboxamide |
| 2410 | 3-{5-[(1,3-benzodioxol-5-ylcarbonyl)amino]-2-hydroxyphenyl}-5-cyclopropyl-N- |
| | (4-methoxybenzyl)-1H-pyrazole-1-carboxamide |
| 2411 | 3-{5-[(1,3-benzodioxol-5-ylcarbonyl)amino]-2-hydroxyphenyl}-N-(sec-butyl)-5- |
| | cyclopropyl-1H-pyrazole-1-carboxamide |
| 2412 | 3-{5-[(1,3-benzodioxol-5-ylcarbonyl)amino]-2-hydroxyphenyl}-5-cyclopropyl-N- |
| | (2-fluorobenzyl)-1H-pyrazole-1-carboxamide |
| 2413 | 3-{5-[(1,3-benzodioxol-5-ylcarbonyl)amino]-2-hydroxyphenyl}-5-cyclopropyl-N- |
| | (3-fluorobenzyl)-1H-pyrazole-1-carboxamide |
| 2414 | 3-{5-[(1,3-benzodioxol-5-ylcarbonyl)amino]-2-hydroxyphenyl}-N,5- |
| | dicyclopropyl-1H-pyrazole-1-carboxamide |
| 2415 | 3-{5-[(1,3-benzodioxol-5-ylcarbonyl)amino]-2-hydroxyphenyl}-5-cyclobutyl-N- |
| | isopropyl-1H-pyrazole-1-carboxamide |
| 2416 | 3-{5-[(1,3-benzodioxol-5-ylcarbonyl)amino]-2-hydroxyphenyl}-5-cyclobutyl-N- |
| | ethyl-1H-pyrazole-1-carboxamide |
| 2417 | N-allyl-3-{5-[(1,3-benzodioxol-5-ylcarbonyl)amino]-2-hydroxyphenyl}-5- |
| | cyclobutyl-1H-pyrazole-1-carboxamide |
| 2418 | 3-{5-[(1,3-benzodioxol-5-ylcarbonyl)amino]-2-hydroxyphenyl}-5-cyclobutyl-N- |
| | propyl-1H-pyrazole-1-carboxamide |
| 2419 | 3-{5-[(1,3-benzodioxol-5-ylcarbonyl)amino]-2-hydroxyphenyl}-N-benzyl-5- |
| | cyclobutyl-1H-pyrazole-1-carboxamide |
| 2420 | 3-{5-[(1,3-benzodioxol-5-ylcarbonyl)amino]-2-hydroxyphenyl}-5-cyclobutyl-N- |
| | (2-phenylethyl)-1H-pyrazole-1-carboxamide |
| 2421 | 3-{5-[(1,3-benzodioxol-5-ylcarbonyl)amino]-2-hydroxyphenyl}-5-cyclobutyl-N- |
| | (2-methylbenzyl)-1H-pyrazole-1-carboxamide |
| 2422 | 3-{5-[(1,3-benzodioxol-5-ylcarbonyl)amino]-2-hydroxyphenyl}-5-cyclobutyl-N- |
| | (3-methylbenzyl)-1H-pyrazole-1-carboxamide |
| 2423 | 3-{5-[(1,3-benzodioxol-5-ylcarbonyl)amino]-2-hydroxyphenyl}-5-cyclobutyl-N- |
| | (4-methylbenzyl)-1H-pyrazole-1-carboxamide |
| 2424 | 3-{5-[(1,3-benzodioxol-5-ylcarbonyl)amino]-2-hydroxyphenyl}-N-(2- |
| | chlorobenzyl)-5-cyclobutyl-1H-pyrazole-1-carboxamide |
| 2425 | 3-{5-[(1,3-benzodioxol-5-ylcarbonyl)amino]-2-hydroxyphenyl}-5-cyclobutyl-N- |
| | (4-fluorobenzyl)-1H-pyrazole-1-carboxamide |
| 2426 | 3-{5-[(1,3-benzodioxol-5-ylcarbonyl)amino]-2-hydroxyphenyl}-5-cyclobutyl-N- |
| | (3,4-dichlorobenzyl)-1H-pyrazole-1-carboxamide |
| 2427 | 3-{5-[(1,3-benzodioxol-5-ylcarbonyl)amino]-2-hydroxyphenyl}-5-cyclobutyl-N- |
| | (4-methoxybenzyl)-1H-pyrazole-1-carboxamide |
| 2428 | 3-{5-[(1,3-benzodioxol-5-ylcarbonyl)amino]-2-hydroxyphenyl}-N-(sec-butyl)-5- |
| | cyclobutyl-1H-pyrazole-1-carboxamide |
| 2429 | 3-{5-[(1,3-benzodioxol-5-ylcarbonyl)amino]-2-hydroxyphenyl}-5-cyclobutyl-N- |
| | (2-fluorobenzyl)-1H-pyrazole-1-carboxamide |
| 2430 | 3-{5-[(1,3-benzodioxol-5-ylcarbonyl)amino]-2-hydroxyphenyl}-5-cyclobutyl-N- |
| | (3-fluorobenzyl)-1H-pyrazole-1-carboxamide |
| 2431 | 3-{5-[(1,3-benzodioxol-5-ylcarbonyl)amino]-2-hydroxyphenyl}-5-cyclobutyl-N- |
| | cyclopropyl-1H-pyrazole-1-carboxamide |
| 2432 | 3-{5-[(1,3-benzodioxol-5-ylcarbonyl)amino]-2-hydroxyphenyl}-5-cyclopentyl-N- |
| | isopropyl-1H-pyrazole-1-carboxamide |
| 2433 | 3-{5-[(1,3-benzodioxol-5-ylcarbonyl)amino]-2-hydroxyphenyl}-5-cyclopentyl-N- |
| | ethyl-1H-pyrazole-1-carboxamide |
| 2434 | N-allyl-3-{5-[(1,3-benzodioxol-5-ylcarbonyl)amino]-2-hydroxyphenyl}-5- |
| | cyclopentyl-1H-pyrazole-1-carboxamide |
| 2435 | 3-{5-[(1,3-benzodioxol-5-ylcarbonyl)amino]-2-hydroxyphenyl}-5-cyclopentyl-N- |
| | propyl-1H-pyrazole-1-carboxamide |
| 2436 | 3-{5-[(1,3-benzodioxol-5-ylcarbonyl)amino]-2-hydroxyphenyl}-N-benzyl-5- |
| | cyclopentyl-1H-pyrazole-1-carboxamide |
| 2437 | 3-{5-[(1,3-benzodioxol-5-ylcarbonyl)amino]-2-hydroxyphenyl}-5-cyclopentyl-N- |
| | (2-phenylethyl)-1H-pyrazole-1-carboxamide |
| 2438 | 3-{5-[(1,3-benzodioxol-5-ylcarbonyl)amino]-2-hydroxyphenyl}-5-cyclopentyl-N- |
| | (2-methylbenzyl)-1H-pyrazole-1-carboxamide |
| 2439 | 3-{5-[(1,3-benzodioxol-5-ylcarbonyl)amino]-2-hydroxyphenyl}-5-cyclopentyl-N- |
| | (3-methylbenzyl)-1H-pyrazole-1-carboxamide |
| 2440 | 3-{5-[(1,3-benzodioxol-5-ylcarbonyl)amino]-2-hydroxyphenyl}-5-cyclopentyl-N- |
| | (4-methylbenzyl)-1H-pyrazole-1-carboxamide |
| 2441 | 3-{5-[(1,3-benzodioxol-5-ylcarbonyl)amino]-2-hydroxyphenyl}-N-(2- |
| | chlorobenzyl)-5-cyclopentyl-1H-pyrazole-1-carboxamide |
| 2442 | 3-{5-[(1,3-benzodioxol-5-ylcarbonyl)amino]-2-hydroxyphenyl}-5-cyclopentyl-N- |
| | (4-fluorobenzyl)-1H-pyrazole-1-carboxamide |
| 2443 | 3-{5-[(1,3-benzodioxol-5-ylcarbonyl)amino]-2-hydroxyphenyl}-5-cyclopentyl-N- |
| | (3,4-dichlorobenzyl)-1H-pyrazole-1-carboxamide |
| 2444 | 3-{5-[(1,3-benzodioxol-5-ylcarbonyl)amino]-2-hydroxyphenyl}-5-cyclopentyl-N- |
| | (4-methoxybenzyl)-1H-pyrazole-1-carboxamide |
| 2445 | 3-{5-[(1,3-benzodioxol-5-ylcarbonyl)amino]-2-hydroxyphenyl}-N-(sec-butyl)-5- |
| | cyclopentyl-1H-pyrazole-1-carboxamide |
| 2446 | 3-{5-[(1,3-benzodioxol-5-ylcarbonyl)amino]-2-hydroxyphenyl}-5-cyclopentyl-N- |
| | (2-fluorobenzyl)-1H-pyrazole-1-carboxamide |
| 2447 | 3-{5-[(1,3-benzodioxol-5-ylcarbonyl)amino]-2-hydroxyphenyl}-5-cyclopentyl-N- |
| | (3-fluorobenzyl)-1H-pyrazole-1-carboxamide |
| 2448 | 3-{5-[(1,3-benzodioxol-5-ylcarbonyl)amino]-2-hydroxyphenyl}-5-cyclopentyl-N- |
| | cyclopropyl-1H-pyrazole-1-carboxamide |
| 2449 | 5-cyclopropyl-3-{5-[(2-ethylbutanoyl)amino]-2-hydroxyphenyl}-N-isopropyl-1H- |
| | pyrazole-1-carboxamide |
| 2450 | 5-cyclopropyl-N-ethyl-3-{5-[(2-ethylbutanoyl)amino]-2-hydroxyphenyl}-1H- |
| | pyrazole-1-carboxamide |
| 2451 | N-allyl-5-cyclopropyl-3-{5-[(2-ethylbutanoyl)amino]-2-hydroxyphenyl}-1H- |
| | pyrazole-1-carboxamide |
| 2452 | 5-cyclopropyl-3-{5-[(2-ethylbutanoyl)amino]-2-hydroxyphenyl}-N-propyl-1H- |
| | pyrazole-1-carboxamide |
| 2453 | N-benzyl-5-cyclopropyl-3-{5-[(2-ethylbutanoyl)amino]-2-hydroxyphenyl}-1H- |
| | pyrazole-1-carboxamide |
| 2454 | 5-cyclopropyl-3-{5-[(2-ethylbutanoyl)amino]-2-hydroxyphenyl}-N-(2- |
| | phenylethyl)-1H-pyrazole-1-carboxamide |
| 2455 | 5-cyclopropyl-3-{5-[(2-ethylbutanoyl)amino]-2-hydroxyphenyl}-N-(2- |
| | methylbenzyl)-1H-pyrazole-1-carboxamide |
| 2456 | 5-cyclopropyl-3-{5-[(2-ethylbutanoyl)amino]-2-hydroxyphenyl}-N-(3- |
| | methylbenzyl)-1H-pyrazole-1-carboxamide |
| 2457 | 5-cyclopropyl-3-{5-[(2-ethylbutanoyl)amino]-2-hydroxyphenyl}-N-(4- |
| | methylbenzyl)-1H-pyrazole-1-carboxamide |
| 2458 | N-(2-chlorobenzyl)-5-cyclopropyl-3-{5-[(2-ethylbutanoyl)amino]-2- |
| | hydroxyphenyl}-1H-pyrazole-1-carboxamide |
| 2459 | 5-cyclopropyl-3-{5-[(2-ethylbutanoyl)amino]-2-hydroxyphenyl}-N-(4- |
| | fluorobenzyl)-1H-pyrazole-1-carboxamide |
| 2460 | 5-cyclopropyl-N-(3,4-dichlorobenzyl)-3-{5-[(2-ethylbutanoyl)amino]-2- |
| | hydroxyphenyl}-1H-pyrazole-1-carboxamide |
| 2461 | 5-cyclopropyl-3-{5-[(2-ethylbutanoyl)amino]-2-hydroxyphenyl}-N-(4- |
| | methoxybenzyl)-1H-pyrazole-1-carboxamide |
| 2462 | N-(sec-butyl)-5-cyclopropyl-3-{5-[(2-ethylbutanoyl)amino]-2-hydroxyphenyl}- |
| | 1H-pyrazole-1-carboxamide |
| 2463 | 5-cyclopropyl-3-{5-[(2-ethylbutanoyl)amino]-2-hydroxyphenyl}-N-(2- |
| | fluorobenzyl)-1H-pyrazole-1-carboxamide |
| 2464 | 5-cyclopropyl-3-{5-[(2-ethylbutanoyl)amino]-2-hydroxyphenyl}-N-(3- |
| | fluorobenzyl)-1H-pyrazole-1-carboxamide |
| 2465 | N,5-dicyclopropyl-3-{5-[(2-ethylbutanoyl)amino]-2-hydroxyphenyl}-1H- |
| | pyrazole-1-carboxamide |
| 2466 | 5-cyclobutyl-3-{5-[(2-ethylbutanoyl)amino]-2-hydroxyphenyl}-N-isopropyl-1H- |
| | pyrazole-1-carboxamide |
| 2467 | 5-cyclobutyl-N-ethyl-3-{5-[(2-ethylbutanoyl)amino]-2-hydroxyphenyl}-1H- |
| | pyrazole-1-carboxamide |
| 2468 | N-allyl-5-cyclobutyl-3-{5-[(2-ethylbutanoyl)amino]-2-hydroxyphenyl}-1H- |
| | pyrazole-1-carboxamide |
| 2469 | 5-cyclobutyl-3-{5-[(2-ethylbutanoyl)amino]-2-hydroxyphenyl}-N-propyl-1H- |
| | pyrazole-1-carboxamide |
| 2470 | N-benzyl-5-cyclobutyl-3-{5-[(2-ethylbutanoyl)amino]-2-hydroxyphenyl}-1H- |
| | pyrazole-1-carboxamide |
| 2471 | 5-cyclobutyl-3-{5-[(2-ethylbutanoyl)amino]-2-hydroxyphenyl}-N-(2- |
| | phenylethyl)-1H-pyrazole-1-carboxamide |
| 2472 | 5-cyclobutyl-3-{5-[(2-ethylbutanoyl)amino]-2-hydroxyphenyl}-N-(2- |
| | methylbenzyl)-1H-pyrazole-1-carboxamide |
| 2473 | 5-cyclobutyl-3-{5-[(2-ethylbutanoyl)amino]-2-hydroxyphenyl}-N-(3- |
| | methylbenzyl)-1H-pyrazole-1-carboxamide |
| 2474 | 5-cyclobutyl-3-{5-[(2-ethylbutanoyl)amino]-2-hydroxyphenyl}-N-(4- |
| | methylbenzyl)-1H-pyrazole-1-carboxamide |
| 2475 | N-(2-chlorobenzyl)-5-cyclobutyl-3-{5-[(2-ethylbutanoyl)amino]-2- |
| | hydroxyphenyl}-1H-pyrazole-1-carboxamide |
| 2476 | 5-cyclobutyl-3-{5-[(2-ethylbutanoyl)amino]-2-hydroxyphenyl}-N-(4- |
| | fluorobenzyl)-1H-pyrazole-1-carboxamide |
| 2477 | 5-cyclobutyl-N-(3,4-dichlorobenzyl)-3-{5-[(2-ethylbutanoyl)amino]-2- |
| | hydroxyphenyl}-1H-pyrazole-1-carboxamide |
| 2478 | 5-cyclobutyl-3-{5-[(2-ethylbutanoyl)amino]-2-hydroxyphenyl}-N-(4- |
| | methoxybenzyl)-1H-pyrazole-1-carboxamide |
| 2479 | N-(sec-butyl)-5-cyclobutyl-3-{5-[(2-ethylbutanoyl)amino]-2-hydroxyphenyl}-1H- |
| | pyrazole-1-carboxamide |
| 2480 | 5-cyclobutyl-3-{5-[(2-ethylbutanoyl)amino]-2-hydroxyphenyl}-N-(2- |
| | fluorobenzyl)-1H-pyrazole-1-carboxamide |
| 2481 | 5-cyclobutyl-3-{5-[(2-ethylbutanoyl)amino]-2-hydroxyphenyl}-N-(3- |
| | fluorobenzyl)-1H-pyrazole-1-carboxamide |
| 2482 | 5-cyclobutyl-N-cyclopropyl-3-{5-[(2-ethylbutanoyl)amino]-2-hydroxyphenyl}- |
| | 1H-pyrazole-1-carboxamide |
| 2483 | 5-cyclopentyl-3-{5-[(2-ethylbutanoyl)amino]-2-hydroxyphenyl}-N-isopropyl-1H- |
| | pyrazole-1-carboxamide |
| 2484 | 5-cyclopentyl-N-ethyl-3-{5-[(2-ethylbutanoyl)amino]-2-hydroxyphenyl}-1H- |
| | pyrazole-1-carboxamide |
| 2485 | N-allyl-5-cyclopentyl-3-{5-[(2-ethylbutanoyl)amino]-2-hydroxyphenyl}-1H- |
| | pyrazole-1-carboxamide |
| 2486 | 5-cyclopentyl-3-{5-[(2-ethylbutanoyl)amino]-2-hydroxyphenyl}-N-propyl-1H- |
| | pyrazole-1-carboxamide |
| 2487 | N-benzyl-5-cyclopentyl-3-{5-[(2-ethylbutanoyl)amino]-2-hydroxyphenyl}-1H- |
| | pyrazole-1-carboxamide |
| 2488 | 5-cyclopentyl-3-{5-[(2-ethylbutanoyl)amino]-2-hydroxyphenyl}-N-(2- |
| | phenylethyl)-1H-pyrazole-1-carboxamide |
| 2489 | 5-cyclopentyl-3-{5-[(2-ethylbutanoyl)amino]-2-hydroxyphenyl}-N-(2- |
| | methylbenzyl)-1H-pyrazole-1-carboxamide |
| 2490 | 5-cyclopentyl-3-{5-[(2-ethylbutanoyl)amino]-2-hydroxyphenyl}-N-(3- |
| | methylbenzyl)-1H-pyrazole-1-carboxamide |
| 2491 | 5-cyclopentyl-3-{5-[(2-ethylbutanoyl)amino]-2-hydroxyphenyl}-N-(4- |
| | methylbenzyl)-1H-pyrazole-1-carboxamide |
| 2492 | 5-cyclopentyl-3-{5-[(2-ethylbutanoyl)amino]-2-hydroxyphenyl}-N-(4- |
| | fluorobenzyl)-1H-pyrazole-1-carboxamide |
| 2493 | 5-cyclopentyl-3-{5-[(2-ethylbutanoyl)amino]-2-hydroxyphenyl}-N-(4- |
| | fluorobenzyl)-1H-pyrazole-1-carboxamide |
| 2494 | 5-cyclopentyl-N-(3,4-dichlorobenzyl)-3-{5-[(2-ethylbutanoyl)amino]-2- |
| | hydroxyphenyl}-1H-pyrazole-1-carboxamide |
| 2495 | 5-cyclopentyl-3-{5-[(2-ethylbutanoyl)amino]-2-hydroxyphenyl}-N-(4- |
| | methoxybenzyl)-1H-pyrazole-1-carboxamide |
| 2496 | N-(sec-butyl)-5-cyclopentyl-3-{5-[(2-ethylbutanoyl)amino]-2-hydroxyphenyl}- |
| | 1H-pyrazole-1-carboxamide |
| 2497 | 5-cyclopentyl-3-{5-[(2-ethylbutanoyl)amino]-2-hydroxyphenyl}-N-(2- |
| | fluorobenzyl)-1H-pyrazole-1-carboxamide |
| 2498 | 5-cyclopentyl-3-{5-[(2-ethylbutanoyl)amino]-2-hydroxyphenyl}-N-(3- |
| | fluorobenzyl)-1H-pyrazole-1-carboxamide |
| 2499 | 5-cyclopentyl-N-cyclopropyl-3-{5-[(2-ethylbutanoyl)amino]-2-hydroxyphenyl}- |
| | 1H-pyrazole-1-carboxamide |
| 2500 | 3-{5-[(1-benzothien-2-ylcarbonyl)amino]-2-hydroxyphenyl}-5-cyclopropyl-N- |
| | isopropyl-1H-pyrazole-1-carboxamide |
| 2501 | 3-{5-[(1-benzothien-2-ylcarbonyl)amino]-2-hydroxyphenyl}-5-cyclopropyl-N- |
| | ethyl-1H-pyrazole-1-carboxamide |
| 2502 | N-allyl-3-{5-[(1-benzothien-2-ylcarbonyl)amino]-2-hydroxyphenyl}-5- |
| | cyclopropyl-1H-pyrazole-1-carboxamide |
| 2503 | 3-{5-[(1-benzothien-2-ylcarbonyl)amino]-2-hydroxyphenyl}-5-cyclopropyl-N- |
| | propyl-1H-pyrazole-1-carboxamide |
| 2504 | 3-{5-[(1-benzothien-2-ylcarbonyl)amino]-2-hydroxyphenyl}-N-benzyl-5- |
| | cyclopropyl-1H-pyrazole-1-carboxamide |
| 2505 | 3-{5-[(1-benzothien-2-ylcarbonyl)amino]-2-hydroxyphenyl}-5-cyclopropyl-N-(2- |
| | phenylethyl)-1H-pyrazole-1-carboxamide |
| 2506 | 3-{5-[(1-benzothien-2-ylcarbonyl)amino]-2-hydroxyphenyl}-5-cyclopropyl-N-(2- |
| | methylbenzyl)-1H-pyrazole-1-carboxamide |
| 2507 | 3-{5-[(1-benzothien-2-ylcarbonyl)amino]-2-hydroxyphenyl}-5-cyclopropyl-N-(3- |
| | methylbenzyl)-1H-pyrazole-1-carboxamide |
| 2508 | 3-{5-[(1-benzothien-2-ylcarbonyl)amino]-2-hydroxyphenyl}-5-cyclopropyl-N-(4- |
| | methylbenzyl)-1H-pyrazole-1-carboxamide |
| 2509 | 3-{5-[(1-benzothien-2-ylcarbonyl)amino]-2-hydroxyphenyl}-N-(2-chlorobenzyl)- |
| | 5-cyclopropyl-1H-pyrazole-1-carboxamide |
| 2510 | 3-{5-[(1-benzothien-2-ylcarbonyl)amino]-2-hydroxyphenyl}-5-cyclopropyl-N-(4- |
| | fluorobenzyl)-1H-pyrazole-1-carboxamide |
| 2511 | 3-{5-[(1-benzothien-2-ylcarbonyl)amino]-2-hydroxyphenyl}-5-cyclopropyl-N- |
| | (3,4-dichlorobenzyl)-1H-pyrazole-1-carboxamide |
| 2512 | 3-{5-[(1-benzothien-2-ylcarbonyl)amino]-2-hydroxyphenyl}-5-cyclopropyl-N-(4- |
| | methoxybenzyl)-1H-pyrazole-1-carboxamide |
| 2513 | 3-{5-[(1-benzothien-2-ylcarbonyl)amino]-2-hydroxyphenyl}-N-(sec-butyl)-5- |
| | cyclopropyl-1H-pyrazole-1-carboxamide |
| 2514 | 3-{5-[(1-benzothien-2-ylcarbonyl)amino]-2-hydroxyphenyl}-5-cyclopropyl-N-(2- |
| | fluorobenzyl)-1H-pyrazole-1-carboxamide |
| 2515 | 3-{5-[(1-benzothien-2-ylcarbonyl)amino]-2-hydroxyphenyl}-5-cyclopropyl-N-(3- |
| | fluorobenzyl)-1H-pyrazole-1-carboxamide |
| 2516 | 3-{5-[(1-benzothien-2-ylcarbonyl)amino]-2-hydroxyphenyl}-N,5-dicyclopropyl- |
| | 1H-pyrazole-1-carboxamide |
| 2517 | 3-{5-[(1-benzothien-2-ylcarbonyl)amino]-2-hydroxyphenyl}-5-cyclobutyl-N- |
| | isopropyl-1H-pyrazole-1-carboxamide |
| 2518 | 3-{5-[(1-benzothien-2-ylcarbonyl)amino]-2-hydroxyphenyl}-5-cyclobutyl-N- |
| | ethyl-1H-pyrazole-1-carboxamide |
| 2519 | N-allyl-3-{5-[(1-benzothien-2-ylcarbonyl)amino]-2-hydroxyphenyl}-5- |
| | cyclobutyl-1H-pyrazole-1-carboxamide |
| 2520 | 3-{5-[(1-benzothien-2-ylcarbonyl)amino]-2-hydroxyphenyl}-5-cyclobutyl-N- |
| | propyl-1H-pyrazole-1-carboxamide |
| 2521 | 3-{5-[(1-benzothien-2-ylcarbonyl)amino]-2-hydroxyphenyl}-N-benzyl-5- |
| | cyclobutyl-1H-pyrazole-1-carboxamide |
| 2522 | 3-{5-[(1-benzothien-2-ylcarbonyl)amino]-2-hydroxyphenyl}-5-cyclobutyl-N-(2- |
| | phenylethyl)-1H-pyrazole-1-carboxamide |
| 2523 | 3-{5-[(1-benzothien-2-ylcarbonyl)amino]-2-hydroxyphenyl}-5-cyclobutyl-N-(2- |
| | methylbenzyl)-1H-pyrazole-1-carboxamide |
| 2524 | 3-{5-[(1-benzothien-2-ylcarbonyl)amino]-2-hydroxyphenyl}-5-cyclobutyl-N-(3- |
| | methylbenzyl)-1H-pyrazole-1-carboxamide |
| 2525 | 3-{5-[(1-benzothien-2-ylcarbonyl)amino]-2-hydroxyphenyl}-5-cyclobutyl-N-(4- |
| | methylbenzyl)-1H-pyrazole-1-carboxamide |
| 2526 | 3-{5-[(1-benzothien-2-ylcarbonyl)amino]-2-hydroxyphenyl}-N-(2-chlorobenzyl)- |
| | 5-cyclobutyl-1H-pyrazole-1-carboxamide |
| 2527 | 3-{5-[(1-benzothien-2-ylcarbonyl)amino]-2-hydroxyphenyl}-5-cyclobutyl-N-(4- |
| | fluorobenzyl)-1H-pyrazole-1-carboxamide |
| 2528 | 3-{5-[(1-benzothien-2-ylcarbonyl)amino]-2-hydroxyphenyl}-5-cyclobutyl-N-(3,4- |
| | dichlorobenzyl)-1H-pyrazole-1-carboxamide |
| 2529 | 3-{5-[(1-benzothien-2-ylcarbonyl)amino]-2-hydroxyphenyl}-5-cyclobutyl-N-(4- |
| | methoxybenzyl)-1H-pyrazole-1-carboxamide |
| 2530 | 3-{5-[(1-benzothien-2-ylcarbonyl)amino]-2-hydroxyphenyl}-N-(sec-butyl)-5- |
| | cyclobutyl-1H-pyrazole-1-carboxamide |
| 2531 | 3-{5-[(1-benzothien-2-ylcarbonyl)amino]-2-hydroxyphenyl}-5-cyclobutyl-N-(2- |
| | fluorobenzyl)-1H-pyrazole-1-carboxamide |
| 2532 | 3-{5-[(1-benzothien-2-ylcarbonyl)amino]-2-hydroxyphenyl}-5-cyclobutyl-N-(3- |
| | fluorobenzyl)-1H-pyrazole-1-carboxamide |
| 2533 | 3-{5-[(1-benzothien-2-ylcarbonyl)amino]-2-hydroxyphenyl}-5-cyclobutyl-N- |
| | cyclopropyl-1H-pyrazole-1-carboxamide |
| 2534 | 3-{5-[(1-benzothien-2-ylcarbonyl)amino]-2-hydroxyphenyl}-5-cyclopentyl-N- |
| | isopropyl-1H-pyrazole-1-carboxamide |
| 2535 | 3-{5-[(1-benzothien-2-ylcarbonyl)amino]-2-hydroxyphenyl}-5-cyclopentyl-N- |
| | ethyl-1H-pyrazole-1-carboxamide |
| 2536 | N-allyl-3-{5-[(1-benzothien-2-ylcarbonyl)amino]-2-hydroxyphenyl}-5- |
| | cyclopentyl-1H-pyrazole-1-carboxamide |
| 2537 | 3-{5-[(1-benzothien-2-ylcarbonyl)amino]-2-hydroxyphenyl}-5-cyclopentyl-N- |
| | propyl-1H-pyrazole-1-carboxamide |
| 2538 | 3-{5-[(1-benzothien-2-ylcarbonyl)amino]-2-hydroxyphenyl}-N-benzyl-5- |
| | cyclopentyl-1H-pyrazole-1-carboxamide |
| 2539 | 3-{5-[(1-benzothien-2-ylcarbonyl)amino]-2-hydroxyphenyl}-5-cyclopentyl-N-(2- |
| | phenylethyl)-1H-pyrazole-1-carboxamide |
| 2540 | 3-{5-[(1-benzothien-2-ylcarbonyl)amino]-2-hydroxyphenyl}-5-cyclopentyl-N-(2- |
| | methylbenzyl)-1H-pyrazole-1-carboxamide |
| 2541 | 3-{5-[(1-benzothien-2-ylcarbonyl)amino]-2-hydroxyphenyl}-5-cyclopentyl-N-(3- |
| | methylbenzyl)-1H-pyrazole-1-carboxamide |
| 2542 | 3-{5-[(1-benzothien-2-ylcarbonyl)amino]-2-hydroxyphenyl}-5-cyclopentyl-N-(4- |
| | methylbenzyl)-1H-pyrazole-1-carboxamide |
| 2543 | 3-{5-[(1-benzothien-2-ylcarbonyl)amino]-2-hydroxyphenyl}-N-(2-chlorobenzyl)- |
| | 5-cyclopentyl-1H-pyrazole-1-carboxamide |
| 2544 | 3-{5-[(1-benzothien-2-ylcarbonyl)amino]-2-hydroxyphenyl}-5-cyclopentyl-N-(4- |
| | fluorobenzyl)-1H-pyrazole-1-carboxamide |
| 2545 | 3-{5-[(1-benzothien-2-ylcarbonyl)amino]-2-hydroxyphenyl}-5-cyclopentyl-N- |
| | (3,4-dichlorobenzyl)-1H-pyrazole-1-carboxamide |
| 2546 | 3-{5-[(1-benzothien-2-ylcarbonyl)amino]-2-hydroxyphenyl}-5-cyclopentyl-N-(4- |
| | methoxybenzyl)-1H-pyrazole-1-carboxamide |
| 2547 | 3-{5-[(1-benzothien-2-ylcarbonyl)amino]-2-hydroxyphenyl}-N-(sec-butyl)-5- |
| | cyclopentyl-1H-pyrazole-1-carboxamide |
| 2548 | 3-{5-[(1-benzothien-2-ylcarbonyl)amino]-2-hydroxyphenyl}-5-cyclopentyl-N-(2- |
| | fluorobenzyl)-1H-pyrazole-1-carboxamide |
| 2549 | 3-{5-[(1-benzothien-2-ylcarbonyl)amino]-2-hydroxyphenyl}-5-cyclopentyl-N-(3- |
| | fluorobenzyl)-1H-pyrazole-1-carboxamide |
| 2550 | 3-{5-[(1-benzothien-2-ylcarbonyl)amino]-2-hydroxyphenyl}-5-cyclopentyl-N- |
| | cyclopropyl-1H-pyrazole-1-carboxamide |
| 2551 | 2-chloro-N-(3-{5-cyclopropyl-1-[(isopropylamino)carbonyl]-1H-pyrazol-3-yl}-4- |
| | hydroxyphenyl)nicotinamide |
| 2552 | 2-chloro-N-(3-{5-cyclopropyl-1-[(ethylamino)carbonyl]-1H-pyrazol-3-yl}-4- |
| | hydroxyphenyl)nicotinamide |
| 2553 | N-(3-{1-[(allylamino)carbonyl]-5-cyclopropyl-1H-pyrazol-3-yl}-4- |
| | hydroxyphenyl)-2-chloronicotinamide |
| 2554 | 2-chloro-N-(3-{5-cyclopropyl-1-[(propylamino)carbonyl]-1H-pyrazol-3-yl}-4- |
| | hydroxyphenyl)nicotinamide |
| 2555 | N-(3-{1-[(benzylamino)carbonyl]-5-cyclopropyl-1H-pyrazol-3-yl}-4- |
| | hydroxyphenyl)-2-chloronicotinamide |
| 2556 | 2-chloro-N-[3-(5-cyclopropyl-1-{[(2-phenylethyl)amino]carbonyl}-1H-pyrazol-3- |
| | yl)-4-hydroxyphenyl]nicotinamide |
| 2557 | 2-chloro-N-[3-(5-cyclopropyl-1-{[(2-methylbenzyl)amino]carbonyl}-1H-pyrazol- |
| | 3-yl)-4-hydroxyphenyl]nicotinamide |
| 2558 | 2-chloro-N-[3-(5-cyclopropyl-1-{[(3-methylbenzyl)amino]carbonyl}-1H-pyrazol- |
| | 3-yl)-4-hydroxyphenyl]nicotinamide |
| 2559 | 2-chloro-N-[3-(5-cyclopropyl-1-{[(4-methylbenzyl)amino]carbonyl}-1H-pyrazol- |
| | 3-yl)-4-hydroxyphenyl]nicotinamide |
| 2560 | 2-chloro-N-[3-(1-{[(2-chlorobenzyl)amino]carbonyl}-5-cyclopropyl-1H-pyrazol- |
| | 3-yl)-4-hydroxyphenyl]nicotinamide |
| 2561 | 2-chloro-N-[3-(5-cyclopropyl-1-{[(4-fluorobenzyl)amino]carbonyl}-1H-pyrazol- |
| | 3-yl)-4-hydroxyphenyl]nicotinamide |
| 2562 | 2-chloro-N-[3-(5-cyclopropyl-1-{[(3,4-dichlorobenzyl)amino]carbonyl}-1H- |
| | pyrazol-3-yl)-4-hydroxyphenyl]nicotinamide |
| 2563 | 2-chloro-N-[3-(5-cyclopropyl-1-{[(4-methoxybenzyl)amino]carbonyl}-1H- |
| | pyrazol-3-yl)-4-hydroxyphenyl]nicotinamide |
| 2564 | N-(3-{1-[(sec-butylamino)carbonyl]-5-cyclopropyl-1H-pyrazol-3-yl}-4- |
| | hydroxyphenyl)-2-chloronicotinamide |
| 2565 | 2-chloro-N-[3-(5-cyclopropyl-1-{[(2-fluorobenzyl)amino]carbonyl}-1H-pyrazol- |
| | 3-yl)-4-hydroxyphenyl]nicotinamide |
| 2566 | 2-chloro-N-[3-(5-cyclopropyl-1-{[(3-fluorobenzyl)amino]carbonyl}-1H-pyrazol- |
| | 3-yl)-4-hydroxyphenyl]nicotinamide |
| 2567 | 2-chloro-N-(3-{5-cyclopropyl-1-[(cyclopropylamino)carbonyl]-1H-pyrazol-3-yl}- |
| | 4-hydroxyphenyl)nicotinamide |
| 2568 | 2-chloro-N-(3-{5-cyclobutyl-1-[(isopropylamino)carbonyl]-1H-pyrazol-3-yl}-4- |
| | hydroxyphenyl)nicotinamide |
| 2569 | 2-chloro-N-(3-{5-cyclobutyl-1-[(ethylamino)carbonyl]-1H-pyrazol-3-yl}-4- |
| | hydroxyphenyl)nicotinamide |
| 2570 | N-(3-{1-[(allylamino)carbonyl]-5-cyclobutyl-1H-pyrazol-3-yl}-4- |
| | hydroxyphenyl)-2-chloronicotinamide |
| 2571 | 2-chloro-N-(3-{5-cyclobutyl-1-[(propylamino)carbonyl]-1H-pyrazol-3-yl}-4- |
| | hydroxyphenyl)nicotinamide |
| 2572 | N-(3-{1-[(benzylamino)carbonyl]-5-cyclobutyl-1H-pyrazol-3-yl}-4- |
| | hydroxyphenyl)-2-chloronicotinamide |
| 2573 | 2-chloro-N-[3-(5-cyclobutyl-1-{[(2-phenylethyl)amino]carbonyl}-1H-pyrazol-3- |
| | yl)-4-hydroxyphenyl]nicotinamide |
| 2574 | 2-chloro-N-[3-(5-cyclobutyl-1-{[(2-methylbenzyl)amino]carbonyl}-1H-pyrazol- |
| | 3-yl)-4-hydroxyphenyl]nicotinamide |
| 2575 | 2-chloro-N-[3-(5-cyclobutyl-1-{[(3-methylbenzyl)amino]carbonyl}-1H-pyrazol- |
| | 3-yl)-4-hydroxyphenyl]nicotinamide |
| 2576 | 2-chloro-N-[3-(5-cyclobutyl-1-{[(4-methylbenzyl)amino]carbonyl}-1H-pyrazol- |
| | 3-yl)-4-hydroxyphenyl]nicotinamide |
| 2577 | 2-chloro-N-[3-(1-{[(2-chlorobenzyl)amino]carbonyl}-5-cyclobutyl-1H-pyrazol-3- |
| | yl)-4-hydroxyphenyl]nicotinamide |
| 2578 | 2-chloro-N-[3-(5-cyclobutyl-1-{[(4-fluorobenzyl)amino]carbonyl}-1H-pyrazol-3- |
| | yl)-4-hydroxyphenyl]nicotinamide |
| 2579 | 2-chloro-N-[3-(5-cyclobutyl-1-{[(3,4-dichlorobenzyl)amino]carbonyl}-1H- |
| | pyrazol-3-yl)-4-hydroxyphenyl]nicotinamide |
| 2580 | 2-chloro-N-[3-(5-cyclobutyl-1-{[(4-methoxybenzyl)amino]carbonyl}-1H-pyrazol- |
| | 3-yl)-4-hydroxyphenyl]nicotinamide |
| 2581 | N-(3-{1-[(sec-butylamino)carbonyl]-5-cyclobutyl-1H-pyrazol-3-yl}-4- |
| | hydroxyphenyl)-2-chloronicotinamide |
| 2582 | 2-chloro-N-[3-(5-cyclobutyl-1-{[(2-fluorobenzyl)amino]carbonyl}-1H-pyrazol-3- |
| | yl)-4-hydroxyphenyl]nicotinamide |
| 2583 | 2-chloro-N-[3-(5-cyclobutyl-1-{[(3-fluorobenzyl)amino]carbonyl}-1H-pyrazol-3- |
| | yl)-4-hydroxyphenyl]nicotinamide |
| 2584 | 2-chloro-N-(3-{5-cyclobutyl-1-[(cyclopropylamino)carbonyl]-1H-pyrazol-3-yl}- |
| | 4-hydroxyphenyl)nicotinamide |
| 2585 | 2-chloro-N-(3-{5-cyclopentyl-1-[(isopropylamino)carbonyl]-1H-pyrazol-3-yl}-4- |
| | hydroxyphenyl)nicotinamide |
| 2586 | 2-chloro-N-(3-{5-cyclopentyl-1-[(ethylamino)carbonyl]-1H-pyrazol-3-yl}-4- |
| | hydroxyphenyl)nicotinamide |
| 2587 | N-(3-{1-[(allylamino)carbonyl]-5-cyclopentyl-1H-pyrazol-3-yl}-4- |
| | hydroxyphenyl)-2-chloronicotinamide |
| 2588 | 2-chloro-N-(3-{5-cyclopentyl-1-[(propylamino)carbonyl]-1H-pyrazol-3-yl}-4- |
| | hydroxyphenyl)nicotinamide |
| 2589 | N-(3-{1-[(benzylamino)carbonyl]-5-cyclopentyl-1H-pyrazol-3-yl}-4- |
| | hydroxyphenyl)-2-chloronicotinamide |
| 2590 | 2-chloro-N-[3-(5-cyclopentyl-1-{[(2-phenylethyl)amino]carbonyl}-1H-pyrazol-3- |
| | yl)-4-hydroxyphenyl]nicotinamide |
| 2591 | 2-chloro-N-[3-(5-cyclopentyl-1-{[(2-methylbenzyl)amino]carbonyl}-1H-pyrazol- |
| | 3-yl)-4-hydroxyphenyl]nicotinamide |
| 2592 | 2-chloro-N-[3-(5-cyclopentyl-1-{[(3-methylbenzyl)amino]carbonyl}-1H-pyrazol- |
| | 3-yl)-4-hydroxyphenyl]nicotinamide |
| 2593 | 2-chloro-N-[3-(5-cyclopentyl-1-{[(4-methylbenzyl)amino]carbonyl}-1H-pyrazol- |
| | 3-yl)-4-hydroxyphenyl]nicotinamide |
| 2594 | 2-chloro-N-[3-(1-{[(2-chlorobenzyl)amino]carbonyl}-5-cyclopentyl-1H-pyrazol- |
| | 3-yl)-4-hydroxyphenyl]nicotinamide |
| 2595 | 2-chloro-N-[3-(5-cyclopentyl-1-{[(4-fluorobenzyl)amino]carbonyl}-1H-pyrazol- |
| | 3-yl)-4-hydroxyphenyl]nicotinamide |
| 2596 | 2-chloro-N-[3-(5-cyclopentyl-1-{[(3,4-dichlorobenzyl)amino]carbonyl}-1H- |
| | pyrazol-3-yl)-4-hydroxyphenyl]nicotinamide |
| 2597 | 2-chloro-N-[3-(5-cyclopentyl-1-{[(4-methoxybenzyl)amino]carbonyl}-1H- |
| | pyrazol-3-yl)-4-hydroxyphenyl]nicotinamide |
| 2598 | N-(3-{1-[(sec-butylamino)carbonyl]-5-cyclopentyl-1H-pyrazol-3-yl}-4- |
| | hydroxyphenyl)-2-chloronicotinamide |
| 2599 | 2-chloro-N-[3-(5-cyclopentyl-1-{[(2-fluorobenzyl)amino]carbonyl}-1H-pyrazol- |
| | 3-yl)-4-hydroxyphenyl]nicotinamide |
| 2600 | 2-chloro-N-[3-(5-cyclopentyl-1-{[(3-fluorobenzyl)amino]carbonyl}-1H-pyrazol- |
| | 3-yl)-4-hydroxyphenyl]nicotinamide |
| 2601 | 2-chloro-N-(3-{5-cyclopentyl-1-[(cyclopropylamino)carbonyl]-1H-pyrazol-3-yl}- |
| | 4-hydroxyphenyl)nicotinamide |
| 2602 | 5-cyclopropyl-3-(2-hydroxy-5-{[4-(trifluoromethoxy)benzoyl]amino}phenyl)-N- |
| | isopropyl-1H-pyrazole-1-carboxamide |
| 2603 | 5-cyclopropyl-N-ethyl-3-(2-hydroxy-5-{[4- |
| | (trifluoromethoxy)benzoyl]amino}phenyl)-1H-pyrazole-1-carboxamide |
| 2604 | N-allyl-5-cyclopropyl-3-(2-hydroxy-5-{[4- |
| | (trifluoromethoxy)benzoyl]amino}-phenyl)-1H-pyrazole-1-carboxamide |
| 2605 | 5-cyclopropyl-3-(2-hydroxy-5-{[4-(trifluoromethoxy)benzoyl]amino}phenyl)-N- |
| | propyl-1H-pyrazole-1-carboxamide |
| 2606 | N-benzyl-5-cyclopropyl-3-(2-hydroxy-5-{[4- |
| | (trifluoromethoxy)benzoyl]amino}phenyl)-1H-pyrazole-1-carboxamide |
| 2607 | 5-cyclopropyl-3-(2-hydroxy-5-{[4-(trifluoromethoxy)benzoyl]amino}phenyl)-N- |
| | (2-phenylethyl)-1H-pyrazole-1-carboxamide |
| 2608 | 5-cyclopropyl-3-(2-hydroxy-5-{[4-(trifluoromethoxy)benzoyl]amino}phenyl)-N- |
| | (2-methylbenzyl)-1H-pyrazole-1-carboxamide |
| 2609 | 5-cyclopropyl-3-(2-hydroxy-5-{[4-(trifluoromethoxy)benzoyl]amino}phenyl)-N- |
| | (3-methylbenzyl)-1H-pyrazole-1-carboxamide |
| 2610 | 5-cyclopropyl-3-(2-hydroxy-5-{[4-(trifluoromethoxy)benzoyl]amino}phenyl)-N- |
| | (4-methylbenzyl)-1H-pyrazole-1-carboxamide |
| 2611 | N-(2-chlorobenzyl)-5-cyclopropyl-3-(2-hydroxy-5-{[4- |
| | (trifluoromethoxy)benzoyl]amino}phenyl)-1H-pyrazole-1-carboxamide |
| 2612 | 5-cyclopropyl-N-(4-fluorobenzyl)-3-(2-hydroxy-5-{[4- |
| | (trifluoromethoxy)benzoyl]amino}phenyl)-1H-pyrazole-1-carboxamide |
| 2613 | 5-cyclopropyl-N-(3,4-dichlorobenzyl)-3-(2-hydroxy-5-{[4- |
| | (trifluoromethoxy)benzoyl]amino}phenyl)-1H-pyrazole-1-carboxamide |
| 2614 | 5-cyclopropyl-3-(2-hydroxy-5-{[4-(trifluoromethoxy)benzoyl]amino}phenyl)-N- |
| | (4-methoxybenzyl)-1H-pyrazole-1-carboxamide |
| 2615 | N-(sec-butyl)-5-cyclopropyl-3-(2-hydroxy-5-{[4- |
| | (trifluoromethoxy)benzoyl]amino}phenyl)-1H-pyrazole-1-carboxamide |
| 2616 | 5-cyclopropyl-N-(2-fluorobenzyl)-3-(2-hydroxy-5-{[4- |
| | (trifluoromethoxy)benzoyl]amino}phenyl)-1H-pyrazole-1-carboxamide |
| 2617 | 5-cyclopropyl-N-(3-fluorobenzyl)-3-(2-hydroxy-5-{[4- |
| | (trifluoromethoxy)benzoyl]amino}phenyl)-1H-pyrazole-1-carboxamide |
| 2618 | N,5-dicyclopropyl-3-(2-hydroxy-5-{[4- |
| | (trifluoromethoxy)benzoyl]amino}phenyl)-1H-pyrazole-1-carboxamide |
| 2619 | 5-cyclobutyl-3-(2-hydroxy-5-{[4-(trifluoromethoxy)benzoyl]amino}phenyl)-N- |
| | isopropyl-1H-pyrazole-1-carboxamide |
| 2620 | 5-cyclobutyl-N-ethyl-3-(2-hydroxy-5-{[4- |
| | (trifluoromethoxy)benzoyl]amino}phenyl)-1H-pyrazole-1-carboxamide |
| 2621 | N-allyl-5-cyclobutyl-3-(2-hydroxy-5-{[4- |
| | (trifluoromethoxy)benzoyl]amino}phenyl)-1H-pyrazole-1-carboxamide |
| 2622 | 5-cyclobutyl-3-(2-hydroxy-5-{[4-(trifluoromethoxy)benzoyl]amino}phenyl)-N- |
| | propyl-1H-pyrazole-1-carboxamide |
| 2623 | N-benzyl-5-cyclobutyl-3-(2-hydroxy-5-{[4- |
| | (trifluoromethoxy)benzoyl]amino}phenyl)-1H-pyrazole-1-carboxamide |
| 2624 | 5-cyclobutyl-3-(2-hydroxy-5-{[4-(trifluoromethoxy)benzoyl]amino}phenyl)-N- |
| | (2-phenylethyl)-1H-pyrazole-1-carboxamide |
| 2625 | 5-cyclobutyl-3-(2-hydroxy-5-{[4-(trifluoromethoxy)benzoyl]amino}phenyl)-N- |
| | (3-methylbenzyl)-1H-pyrazole-1-carboxamide |
| 2626 | 5-cyclobutyl-3-(2-hydroxy-5-{[4-(trifluoromethoxy)benzoyl]amino}phenyl)-N- |
| | (3-methylbenzyl)-1H-pyrazole-1-carboxamide |
| 2627 | 5-cyclobutyl-3-(2-hydroxy-5-{[4-(trifluoromethoxy)benzoyl]amino}phenyl)-N- |
| | (4-methylbenzyl)-1H-pyrazole-1-carboxamide |
| 2628 | N-(2-chlorobenzyl)-5-cyclobutyl-3-(2-hydroxy-5-{[4- |
| | (trifluoromethoxy)benzoyl]amino}phenyl)-1H-pyrazole-1-carboxamide |
| 2629 | 5-cyclobutyl-N-(4-fluorobenzyl)-3-(2-hydroxy-5-{[4- |
| | (trifluoromethoxy)benzoyl]amino}phenyl)-1H-pyrazole-1-carboxamide |
| 2630 | 5-cyclobutyl-N-(3,4-dichlorobenzyl)-3-(2-hydroxy-5-{[4- |
| | (trifluoromethoxy)benzoyl]amino}phenyl)-1H-pyrazole-1-carboxamide |
| 2631 | 5-cyclobutyl-3-(2-hydroxy-5-{[4-(trifluoromethoxy)benzoyl]amino}phenyl)-N- |
| | (4-methoxybenzyl)-1H-pyrazole-1-carboxamide |
| 2632 | N-(sec-butyl)-5-cyclobutyl-3-(2-hydroxy-5-{[4- |
| | (trifluoromethoxy)benzoyl]amino}phenyl)-1H-pyrazole-1-carboxamide |
| 2633 | 5-cyclobutyl-N-(2-fluorobenzyl)-3-(2-hydroxy-5-{[4- |
| | (trifluoromethoxy)benzoyl]amino}phenyl)-1H-pyrazole-1-carboxamide |
| 2634 | 5-cyclobutyl-N-(3-fluorobenzyl)-3-(2-hydroxy-5-{[4- |
| | (trifluoromethoxy)benzoyl]amino}phenyl)-1H-pyrazole-1-carboxamide |
| 2635 | 5-cyclobutyl-N-cyclopropyl-3-(2-hydroxy-5-{[4- |
| | (trifluoromethoxy)benzoyl]amino}phenyl)-1H-pyrazole-1-carboxamide |
| 2636 | 5-cyclopentyl-3-(2-hydroxy-5-{[4-(trifluoromethoxy)benzoyl]amino}phenyl)-N- |
| | isopropyl-1H-pyrazole-1-carboxamide |
| 2637 | 5-cyclopentyl-N-ethyl-3-(2-hydroxy-5-{[4- |
| | (trifluoromethoxy)benzoyl]amino}phenyl)-1H-pyrazole-1-carboxamide |
| 2638 | N-allyl-5-cyclopentyl-3-(2-hydroxy-5-{[4- |
| | (trifluoromethoxy)benzoyl]amino}phenyl)-1H-pyrazole-1-carboxamide |
| 2639 | 5-cyclopentyl-3-(2-hydroxy-5-{[4-(trifluoromethoxy)benzoyl]amino}phenyl)-N- |
| | propyl-1H-pyrazole-1-carboxamide |
| 2640 | N-benzyl-5-cyclopentyl-3-(2-hydroxy-5-{[4- |
| | (trifluoromethoxy)benzoyl]amino}phenyl)-1H-pyrazole-1-carboxamide |
| 2641 | 5-cyclopentyl-3-(2-hydroxy-5-{[4-(trifluoromethoxy)benzoyl]amino}phenyl)-N- |
| | (2-phenylethyl)-1H-pyrazole-1-carboxamide |
| 2642 | 5-cyclopentyl-3-(2-hydroxy-5-{[4-(trifluoromethoxy)benzoyl]amino}phenyl)-N- |
| | (2-methylbenzyl)-1H-pyrazole-1-carboxamide |
| 2643 | 5-cyclopentyl-3-(2-hydroxy-5-{[4-(trifluoromethoxy)benzoyl]amino}phenyl)-N- |
| | (3-methylbenzyl)-1H-pyrazole-1-carboxamide |
| 2644 | 5-cyclopentyl-3-(2-hydroxy-5-{[4-(trifluoromethoxy)benzoyl]amino}phenyl)-N- |
| | (4-methylbenzyl)-1H-pyrazole-1-carboxamide |
| 2645 | N-(2-chlorobenzyl)-5-cyclopentyl-3-(2-hydroxy-5-{[4- |
| | (trifluoromethoxy)benzoyl]amino}phenyl)-1H-pyrazole-1-carboxamide |
| 2646 | 5-cyclopentyl-N-(4-fluorobenzyl)-3-(2-hydroxy-5-{[4- |
| | (trifluoromethoxy)benzoyl]amino}phenyl)-1H-pyrazole-1-carboxamide |
| 2647 | 5-cyclopentyl-N-(3,4-dichlorobenzyl)-3-(2-hydroxy-5-{[4- |
| | (trifluoromethoxy)benzoyl]amino}phenyl)-1H-pyrazole-1-carboxamide |
| 2648 | 5-cyclopentyl-3-(2-hydroxy-5-{[4-(trifluoromethoxy)benzoyl]amino}phenyl)-N- |
| | (4-methoxybenzyl)-1H-pyrazole-1-carboxamide |
| 2649 | N-(sec-butyl)-5-cyclopentyl-3-(2-hydroxy-5-{[4- |
| | (trifluoromethoxy)benzoyl]amino}phenyl)-1H-pyrazole-1-carboxamide |
| 2650 | 5-cyclopentyl-N-(2-fluorobenzyl)-3-(2-hydroxy-5-{[4- |
| | (trifluoromethoxy)benzoyl]amino}phenyl)-1H-pyrazole-1-carboxamide |
| 2651 | 5-cyclopentyl-N-(3-fluorobenzyl)-3-(2-hydroxy-5-{[4- |
| | (trifluoromethoxy)benzoyl]amino}phenyl)-1H-pyrazole-1-carboxamide |
| 2652 | 5-cyclopentyl-N-cyclopropyl-3-(2-hydroxy-5-{[4- |
| | (trifluoromethoxy)benzoyl]amino}phenyl)-1H-pyrazole-1-carboxamide |
| 2653 | N-(3-{5-cyclopropyl-1-[(isopropylamino)carbonyl]-1H-pyrazol-3-yl}-4- |
| | hydroxyphenyl)isoxazole-5-carboxamide |
| 2654 | N-(3-{5-cyclopropyl-1-[(ethylamino)carbonyl]-1H-pyrazol-3-yl}-4- |
| | hydroxyphenyl)isoxazole-5-carboxamide |
| 2655 | N-(3-{1-[(allylamino)carbonyl]-5-cyclopropyl-1H-pyrazol-3-yl}-4- |
| | hydroxyphenyl)isoxazole-5-carboxamide |
| 2656 | N-(3-{5-cyclopropyl-1-[(propylamino)carbonyl]-1H-pyrazol-3-yl}-4- |
| | hydroxyphenyl)isoxazole-5-carboxamide |
| 2657 | N-(3-{1-[(benzylamino)carbonyl]-5-cyclopropyl-1H-pyrazol-3-yl}-4- |
| | hydroxyphenyl)isoxazole-5-carboxamide |
| 2658 | N-[3-(5-cyclopropyl-1-{[(2-phenylethyl)amino]carbonyl}-1H-pyrazol-3-yl)-4- |
| | hydroxyphenyl]isoxazole-5-carboxamide |
| 2659 | N-[3-(5-cyclopropyl-1-{[(2-methylbenzyl)amino]carbonyl}-1H-pyrazol-3-yl)-4- |
| | hydroxyphenyl]isoxazole-5-carboxamide |
| 2660 | N-[3-(5-cyclopropyl-1-{[(3-methylbenzyl)amino]carbonyl}-1H-pyrazol-3-yl)-4- |
| | hydroxyphenyl]isoxazole-5-carboxamide |
| 2661 | N-[3-(5-cyclopropyl-1-{[(4-methylbenzyl)amino]carbonyl}-1H-pyrazol-3-yl)-4- |
| | hydroxyphenyl]isoxazole-5-carboxamide |
| 2662 | N-[3-(1-{[(2-chlorobenzyl)amino]carbonyl}-5-cyclopropyl-1H-pyrazol-3-yl)-4- |
| | hydroxyphenyl]isoxazole-5-carboxamide |
| 2663 | N-[3-(5-cyclopropyl-1-{[(4-fluorobenzyl)amino]carbonyl}-1H-pyrazol-3-yl)-4- |
| | hydroxyphenyl]isoxazole-5-carboxamide |
| 2664 | N-[3-(5-cyclopropyl-1-{[(3,4-dichlorobenzyl)amino]carbonyl}-1H-pyrazol-3-yl)- |
| | 4-hydroxyphenyl]isoxazole-5-carboxamide |
| 2665 | N-[3-(5-cyclopropyl-1-{[(4-methoxybenzyl)amino]carbonyl}-1H-pyrazol-3-yl)-4- |
| | hydroxyphenyl]isoxazole-5-carboxamide |
| 2666 | N-(3-{1-[(sec-butylamino)carbonyl]-5-cyclopropyl-1H-pyrazol-3-yl}-4- |
| | hydroxyphenyl)isoxazole-5-carboxamide |
| 2667 | N-[3-(5-cyclopropyl-1-{[(2-fluorobenzyl)amino]carbonyl}-1H-pyrazol-3-yl)-4- |
| | hydroxyphenyl]isoxazole-5-carboxamide |
| 2668 | N-[3-(5-cyclopropyl-1-{[(3-fluorobenzyl)amino]carbonyl}-1H-pyrazol-3-yl)-4- |
| | hydroxyphenyl]isoxazole-5-carboxamide |
| 2669 | N-(3-{5-cyclopropyl-1-[(cyclopropylamino)carbonyl]-1H-pyrazol-3-yl}-4- |
| | hydroxyphenyl)isoxazole-5-carboxamide |
| 2670 | N-(3-{5-cyclobutyl-1-[(isopropylamino)carbonyl]-1H-pyrazol-3-yl}-4- |
| | hydroxyphenyl)isoxazole-5-carboxamide |
| 2671 | N-(3-{5-cyclobutyl-1-[(ethylamino)carbonyl]-1H-pyrazol-3-yl}-4- |
| | hydroxyphenyl)isoxazole-5-carboxamide |
| 2672 | N-(3-{1-[(allylamino)carbonyl]-5-cyclobutyl-1H-pyrazol-3-yl}-4- |
| | hydroxyphenyl)isoxazole-5-carboxamide |
| 2673 | N-(3-{5-cyclobutyl-1-[(propylamino)carbonyl]-1H-pyrazol-3-yl}-4- |
| | hydroxyphenyl)isoxazole-5-carboxamide |
| 2674 | N-(3-{1-[(benzylamino)carbonyl]-5-cyclobutyl-1H-pyrazol-3-yl}-4- |
| | hydroxyphenyl)isoxazole-5-carboxamide |
| 2675 | N-[3-(5-cyclobutyl-1-{[(2-phenylethyl)amino]carbonyl}-1H-pyrazol-3-yl)-4- |
| | hydroxyphenyl]isoxazole-5-carboxamide |
| 2676 | N-[3-(5-cyclobutyl-1-{[(2-methylbenzyl)amino]carbonyl}-1H-pyrazol-3-yl)-4- |
| | hydroxyphenyl]isoxazole-5-carboxamide |
| 2677 | N-[3-(5-cyclobutyl-1-{[(3-methylbenzyl)amino]carbonyl}-1H-pyrazol-3-yl)-4- |
| | hydroxyphenyl]isoxazole-5-carboxamide |
| 2678 | N-[3-(5-cyclobutyl-1-{[(4-methylbenzyl)amino]carbonyl}-1H-pyrazol-3-yl)-4- |
| | hydroxyphenyl]isoxazole-5-carboxamide |
| 2679 | N-[3-(1-{[(2-chlorobenzyl)amino]carbonyl }-5-cyclobutyl-1H-pyrazol-3-yl)-4- |
| | hydroxyphenyl]isoxazole-5-carboxamide |
| 2680 | N-[3-(5-cyclobutyl-1-{[(4-fluorobenzyl)amino]carbonyl}-1H-pyrazol-3-yl)-4- |
| | hydroxyphenyl]isoxazole-5-carboxamide |
| 2681 | N-[3-(5-cyclobutyl-1-{[(3,4-dichlorobenzyl)amino]carbonyl}-1H-pyrazol-3-yl)-4- |
| | hydroxyphenyl]isoxazole-5-carboxamide |
| 2682 | N-[3-(5-cyclobutyl-1-{[(4-methoxybenzyl)amino]carbonyl}-1H-pyrazol-3-yl)-4- |
| | hydroxyphenyl]isoxazole-5-carboxamide |
| 2683 | N-(3-{1-[(sec-butylamino)carbonyl]-5-cyclobutyl-1H-pyrazol-3-yl}-4- |
| | hydroxyphenyl)isoxazole-5-carboxamide |
| 2684 | N-[3-(5-cyclobutyl-1-{[(2-fluorobenzyl)amino]carbonyl}-1H-pyrazol-3-yl)-4- |
| | hydroxyphenyl]isoxazole-5-carboxamide |
| 2685 | N-[3-(5-cyclobutyl-1-{[(3-fluorobenzyl)amino]carbonyl}-1H-pyrazol-3-yl)-4- |
| | hydroxyphenyl]isoxazole-5-carboxamide |
| 2686 | N-(3-{5-cyclobutyl-1-[(cyclopropylamino)carbonyl]-1H-pyrazol-3-yl}-4- |
| | hydroxyphenyl)isoxazole-5-carboxamide |
| 2687 | N-(3-{5-cyclopentyl-1-[(isopropylamino)carbonyl]-1H-pyrazol-3-yl}-4- |
| | hydroxyphenyl)isoxazole-5-carboxamide |
| 2688 | N-(3-{5-cyclopentyl-1-[(ethylamino)carbonyl]-1H-pyrazol-3-yl}-4- |
| | hydroxyphenyl)isoxazole-5-carboxamide |
| 2689 | N-(3-{1-[(allylamino)carbonyl]-5-cyclopentyl-1H-pyrazol-3-yl}-4- |
| | hydroxyphenyl)isoxazole-5-carboxamide |
| 2690 | N-(3-{5-cyclopentyl-1-[(propylamino)carbonyl]-1H-pyrazol-3-yl}-4- |
| | hydroxyphenyl)isoxazole-5-carboxamide |
| 2691 | N-(3-{1-[(benzylamino)carbonyl]-5-cyclopentyl-1H-pyrazol-3-yl}-4- |
| | hydroxyphenyl)isoxazole-5-carboxamide |
| 2692 | N-[3-(5-cyclopentyl-1-{[(2-phenylethyl)amino]carbonyl}-1H-pyrazol-3-yl)-4- |
| | hydroxyphenyl]isoxazole-5-carboxamide |
| 2693 | N-[3-(5-cyclopentyl-1-{[(2-methylbenzyl)amino]carbonyl}-1H-pyrazol-3-yl)-4- |
| | hydroxyphenyl]isoxazole-5-carboxamide |
| 2694 | N-[3-(5-cyclopentyl-1-{[(3-methylbenzyl)amino]carbonyl}-1H-pyrazol-3-yl)-4- |
| | hydroxyphenyl]isoxazole-5-carboxamide |
| 2695 | N-[3-(5-cyclopentyl-1-{[(4-methylbenzyl)amino]carbonyl}-1H-pyrazol-3-yl)-4- |
| | hydroxyphenyl]isoxazole-5-carboxamide |
| 2696 | N-[3-(1-{[(2-chlorobenzyl)amino]carbonyl}-5-cyclopentyl-1H-pyrazol-3-yl)-4- |
| | hydroxyphenyl]isoxazole-5-carboxamide |
| 2697 | N-[3-(5-cyclopentyl-1-{[(4-fluorobenzyl)amino]carbonyl}-1H-pyrazol-3-yl)-4- |
| | hydroxyphenyl]isoxazole-5-carboxamide |
| 2698 | N-[3-(5-cyclopentyl-1-{[(3,4-dichlorobenzyl)amino]carbonyl}-1H-pyrazol-3-yl)- |
| | 4-hydroxyphenyl]isoxazole-5-carboxamide |
| 2699 | N-[3-(5-cyclopentyl-1-{[(4-methoxybenzyl)amino]carbonyl}-1H-pyrazol-3-yl)-4- |
| | hydroxyphenyl]isoxazole-5-carboxamide |
| 2700 | N-(3-{1-[(sec-butylamino)carbonyl]-5-cyclopentyl-1H-pyrazol-3-yl}-4- |
| | hydroxyphenyl)isoxazole-5-carboxamide |
| 2701 | N-[3-(5-cyclopentyl-1-{[(2-fluorobenzyl)amino]carbonyl}-1H-pyrazol-3-yl)-4- |
| | hydroxyphenyl]isoxazole-5-carboxamide |
| 2702 | N-[3-(5-cyclopentyl-1-{[(3-fluorobenzyl)amino]carbonyl}-1H-pyrazol-3-yl)-4- |
| | hydroxyphenyl]isoxazole-5-carboxamide |
| 2703 | N-(3-{5-cyclopentyl-1-[(cyclopropylamino)carbonyl]-1H-pyrazol-3-yl}-4- |
| | hydroxyphenyl)isoxazole-5-carboxamide |
| 2704 | 5-cyclopropyl-3-(2-hydroxy-5-{[(1-methyl-1H-pyrrol-2- |
| | yl)carbonyl]amino}phenyl)-N-isopropyl-1H-pyrazole-1-carboxamide |
| 2705 | 5-cyclopropyl-N-ethyl-3-(2-hydroxy-5-{[(1-methyl-1H-pyrrol-2- |
| | yl)carbonyl]amino}phenyl)-1H-pyrazole-1-carboxamide |
| 2706 | N-allyl-5-cyclopropyl-3-(2-hydroxy-5-{[(1-methyl-1H-pyrrol-2- |
| | yl)carbonyl]amino}phenyl)-1H-pyrazole-1-carboxamide |
| 2707 | 5-cyclopropyl-3-(2-hydroxy-5-{[(1-methyl-1H-pyrrol-2- |
| | yl)carbonyl]amino}phenyl)-N-propyl-1H-pyrazole-1-carboxamide |
| 2708 | N-benzyl-5-cyclopropyl-3-(2-hydroxy-5-{[(1-methyl-1H-pyrrol-2- |
| | yl)carbonyl]amino}phenyl)-1H-pyrazole-1-carboxamide |
| 2709 | 5-cyclopropyl-3-(2-hydroxy-5-{[(1-methyl-1H-pyrrol-2- |
| | yl)carbonyl]amino}phenyl)-N-(2-phenylethyl)-1H-pyrazole-1-carboxamide |
| 2710 | 5-cyclopropyl-3-(2-hydroxy-5-{[(1-methyl-1H-pyrrol-2- |
| | yl)carbonyl]amino}phenyl)-N-(2-methylbenzyl)-1H-pyrazole-1-carboxamide |
| 2711 | 5-cyclopropyl-3-(2-hydroxy-5-{[(1-methyl-1H-pyrrol-2- |
| | yl)carbonyl]amino}phenyl)-N-(3-methylbenzyl)-1H-pyrazole-1-carboxamide |
| 2712 | 5-cyclopropyl-3-(2-hydroxy-5-{[(1-methyl-1H-pyrrol-2- |
| | yl)carbonyl]amino}phenyl)-N-(4-methylbenzyl)-1H-pyrazole-1-carboxamide |
| 2713 | N-(2-chlorobenzyl)-5-cyclopropyl-3-(2-hydroxy-5-{[(1-methyl-1H-pyrrol-2- |
| | yl)carbonyl]amino}phenyl)-1H-pyrazole-1-carboxamide |
| 2714 | 5-cyclopropyl-N-(4-fluorobenzyl)-3-(2-hydroxy-5-{[(1-methyl-1H-pyrrol-2- |
| | yl)carbonyl]amino}phenyl)-1H-pyrazole-1-carboxamide |
| 2715 | 5-cyclopropyl-N-(3,4-dichlorobenzyl)-3-(2-hydroxy-5-{[(1-methyl-1H-pyrrol-2- |
| | yl)carbonyl]amino}phenyl)-1H-pyrazole-1-carboxamide |
| 2716 | 5-cyclopropyl-3-(2-hydroxy-5-{[(1-methyl-1H-pyrrol-2- |
| | yl)carbonyl]amino}phenyl)-N-(4-methoxybenzyl)-1H-pyrazole-1-carboxamide |
| 2717 | N-(sec-butyl)-5-cyclopropyl-3-(2-hydroxy-5-{[(1-methyl-1H-pyrrol-2- |
| | yl)carbonyl]amino}phenyl)-1H-pyrazole-1-carboxamide |
| 2718 | 5-cyclopropyl-N-(2-fluorobenzyl)-3-(2-hydroxy-5-{[(1-methyl-1H-pyrrol-2- |
| | yl)carbonyl]amino}phenyl)-1H-pyrazole-1-carboxamide |
| 2719 | 5-cyclopropyl-N-(3-fluorobenzyl)-3-(2-hydroxy-5-{[(1-methyl-1H-pyrrol-2- |
| | yl)carbonyl]amino}phenyl)-1H-pyrazole-1-carboxamide |
| 2720 | N,5-dicyclopropyl-3-(2-hydroxy-5-{[(1-methyl-1H-pyrrol-2- |
| | yl)carbonyl]amino}phenyl)-1H-pyrazole-1-carboxamide |
| 2721 | 5-cyclobutyl-3-(2-hydroxy-5-{[(1-methyl-1H-pyrrol-2- |
| | yl)carbonyl]amino}phenyl)-N-isopropyl-1H-pyrazole-1-carboxamide |
| 2722 | 5-cyclobutyl-N-ethyl-3-(2-hydroxy-5-{[(1-methyl-1H-pyrrol-2- |
| | yl)carbonyl]amino}phenyl)-1H-pyrazole-1-carboxamide |
| 2723 | N-allyl-5-cyclobutyl-3-(2-hydroxy-5-{[(1-methyl-1H-pyrrol-2- |
| | yl)carbonyl]amino}phenyl)-1H-pyrazole-1-carboxamide |
| 2724 | 5-cyclobutyl-3-(2-hydroxy-5-{[(1-methyl-1H-pyrrol-2- |
| | yl)carbonyl]amino}phenyl)-N-propyl-1H-pyrazole-1-carboxamide |
| 2725 | N-benzyl-5-cyclobutyl-3-(2-hydroxy-5-{[(1-methyl-1H-pyrrol-2- |
| | yl)carbonyl]amino}phenyl)-1H-pyrazole-1-carboxamide |
| 2726 | 5-cyclobutyl-3-(2-hydroxy-5-{[(1-methyl-1H-pyrrol-2- |
| | yl)carbonyl]amino}phenyl)-N-(2-phenylethyl)-1H-pyrazole-1-carboxamide |
| 2727 | 5-cyclobutyl-3-(2-hydroxy-5-{[(1-methyl-1H-pyrrol-2- |
| | yl)carbonyl]amino}phenyl)-N-(2-methylbenzyl)-1H-pyrazole-1-carboxamide |
| 2728 | 5-cyclobutyl-3-(2-hydroxy-5-{[(1-methyl-1H-pyrrol-2- |
| | yl)carbonyl]amino}phenyl)-N-(3-methylbenzyl)-1H-pyrazole-1-carboxamide |
| 2729 | 5-cyclobutyl-3-(2-hydroxy-5-{[(1-methyl-1H-pyrrol-2- |
| | yl)carbonyl]amino}phenyl)-N-(4-methylbenzyl)-1H-pyrazole-1-carboxamide |
| 2730 | N-(2-chlorobenzyl)-5-cyclobutyl-3-(2-hydroxy-5-{[(1-methyl-1H-pyrrol-2- |
| | yl)carbonyl]amino}phenyl)-1H-pyrazole-1-carboxamide |
| 2731 | 5-cyclobutyl-N-(4-fluorobenzyl)-3-(2-hydroxy-5-{[(1-methyl-1H-pyrrol-2- |
| | yl)carbonyl]amino}phenyl)-1H-pyrazole-1-carboxamide |
| 2732 | 5-cyclobutyl-N-(3,4-dichlorobenzyl)-3-(2-hydroxy-5-{[(1-methyl-1H-pyrrol-2- |
| | yl)carbonyl]amino}phenyl)-1H-pyrazole-1-carboxamide |
| 2733 | 5-cyclobutyl-3-(2-hydroxy-5-{[(1-methyl-1H-pyrrol-2- |
| | yl)carbonyl]amino}phenyl)-N-(4-methoxybenzyl)-1H-pyrazole-1-carboxamide |
| 2734 | N-(sec-butyl)-5-cyclobutyl-3-(2-hydroxy-5-{[(1-methyl-1H-pyrrol-2- |
| | yl)carbonyl]amino}phenyl)-1H-pyrazole-1-carboxamide |
| 2735 | 5-cyclobutyl-N-(2-fluorobenzyl)-3-(2-hydroxy-5-{[(1-methyl-1H-pyrrol-2- |
| | yl)carbonyl]amino}phenyl)-1H-pyrazole-1-carboxamide |
| 2736 | 5-cyclobutyl-N-(3-fluorobenzyl)-3-(2-hydroxy-5-{[(1-methyl-1H-pyrrol-2- |
| | yl)carbonyl]amino}phenyl)-1H-pyrazole-1-carboxamide |
| 2737 | 5-cyclobutyl-N-cyclopropyl-3-(2-hydroxy-5-{[(1-methyl-1H-pyrrol-2- |
| | yl)carbonyl]amino}phenyl)-1H-pyrazole-1-carboxamide |
| 2738 | 5-cyclopentyl-3-(2-hydroxy-5-{[(1-methyl-1H-pyrrol-2- |
| | yl)carbonyl]amino}phenyl)-N-isopropyl-1H-pyrazole-1-carboxamide |
| 2739 | 5-cyclopentyl-N-ethyl-3-(2-hydroxy-5-{[(1-methyl-1H-pyrrol-2- |
| | yl)carbonyl]amino}phenyl)-1H-pyrazole-1-carboxamide |
| 2740 | N-allyl-5-cyclopentyl-3-(2-hydroxy-5-{[(1-methyl-1H-pyrrol-2- |
| | yl)carbonyl]amino}phenyl)-1H-pyrazole-1-carboxamide |
| 2741 | 5-cyclopentyl-3-(2-hydroxy-5-{[(1-methyl-1H-pyrrol-2- |
| | yl)carbonyl]amino}phenyl)-N-propyl-1H-pyrazole-1-carboxamide |
| 2742 | N-benzyl-5-cyclopentyl-3-(2-hydroxy-5-{[(1-methyl-1H-pyrrol-2- |
| | yl)carbonyl]amino}phenyl)-1H-pyrazole-1-carboxamide |
| 2743 | 5-cyclopentyl-3-(2-hydroxy-5-{[(1-methyl-1H-pyrrol-2- |
| | yl)carbonyl]amino}phenyl)-N-(2-phenylethyl)-1H-pyrazole-1-carboxamide |
| 2744 | 5-cyclopentyl-3-(2-hydroxy-5-{[(1-methyl-1H-pyrrol-2- |
| | yl)carbonyl]amino}phenyl)-N-(2-methylbenzyl)-1H-pyrazole-1-carboxamide |
| 2745 | 5-cyclopentyl-3-(2-hydroxy-5-{[(1-methyl-1H-pyrrol-2- |
| | yl)carbonyl]amino}phenyl)-N-(3-methylbenzyl)-1H-pyrazole-1-carboxamide |
| 2746 | 5-cyclopentyl-3-(2-hydroxy-5-{[(1-methyl-1H-pyrrol-2- |
| | yl)carbonyl]amino}phenyl)-N-(4-methylbenzyl)-1H-pyrazole-1-carboxamide |
| 2747 | N-(2-chlorobenzyl)-5-cyclopentyl-3-(2-hydroxy-5-{[(1-methyl-1H-pyrrol-2- |
| | yl)carbonyl]amino}phenyl)-1H-pyrazole-1-carboxamide |
| 2748 | 5-cyclopentyl-N-(4-fluorobenzyl)-3-(2-hydroxy-5-{[(1-methyl-1H-pyrrol-2- |
| | yl)carbonyl]amino}phenyl)-1H-pyrazole-1-carboxamide |
| 2749 | 5-cyclopentyl-N-(3,4-dichlorobenzyl)-3-(2-hydroxy-5-{[(1-methyl-1H-pyrrol-2- |
| | yl)carbonyl]amino}phenyl)-1H-pyrazole-1-carboxamide |
| 2750 | 5-cyclopentyl-3-(2-hydroxy-5-{[(1-methyl-1H-pyrrol-2- |
| | yl)carbonyl]amino}phenyl)-N-(4-methoxybenzyl)-1H-pyrazole-1-carboxamide |
| 2751 | N-(sec-butyl)-5-cyclopentyl-3-(2-hydroxy-5-{[(1-methyl-1H-pyrrol-2- |
| | yl)carbonyl]amino}phenyl)-1H-pyrazole-1-carboxamide |
| 2752 | 5-cyclopentyl-N-(2-fluorobenzyl)-3-(2-hydroxy-5-{[(1-methyl-1H-pyrrol-2- |
| | yl)carbonyl]amino}phenyl)-1H-pyrazole-1-carboxamide |
| 2753 | 5-cyclopentyl-N-(3-fluorobenzyl)-3-(2-hydroxy-5-{[(1-methyl-1H-pyrrol-2- |
| | yl)carbonyl]amino}phenyl)-1H-pyrazole-1-carboxamide |
| 2754 | 5-cyclopentyl-N-cyclopropyl-3-(2-hydroxy-5-{[(1-methyl-1H-pyrrol-2- |
| | yl)carbonyl]amino}phenyl)-1H-pyrazole-1-carboxamide |

21. A process for preparing the compounds of formula (I) or the pharmaceutically acceptable salts thereof, as defined in claim 12, which process comprises:
a) removing Q from a compound of formula (II) 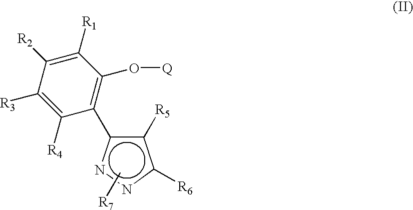
wherein R₁ to R₇ are as defined in claim 12 and Q is a hydroxy-phenyl protecting group or a solid support, so as to obtain the compound of formula (I) as defined above and, if desired,
b) converting the resultant compound of formula (I) into another compound of formula (I) and/or into a salt or free form thereof.

22. A library of two or more compounds of formula (I): 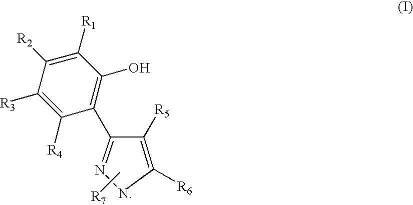
wherein R₁ to R₇ are as defined in claim 12.

23. A pharmaceutical composition comprising a therapeutically effective amount of a compound of formula (I), as defined in claim 12, and at least one pharmaceutically acceptable carrier and/or diluent.

24. A pharmaceutical composition according to claim 23 further comprising one or more chemotherapeutic agents.

25. A product comprising a compound of formula (I) as defined in claim 12 or a pharmaceutical composition thereof as defined in claim 23, and one or more chemotherapeutic agents, as a combined preparation for simultaneous, separate or sequential use in anticancer therapy.

26. A compound of formula (I), as defined in claim 12, for us as a medicament.

27. Use of a compound of formula (I), as defined in claim 12, in the manufacture of a medicament with antitumor activity.
